SEQUENCE LISTING (130 sequences)

SEQ ID NO: 1 - protein, 117 aa, synthetic construct
  REGION 1..117: note = JH1 VH
QVQLVQSGAEVKKPGASVKVSCKASGYTFTSYAMHWVRQAPGQRLEWMGWINAGNGNTKYSQKFQGRVTITRDTSASTAYMELRSLRSDDTAVYYCASGPGRLPVVWGQGTLVTVSS

SEQ ID NO: 2 - protein, 108 aa, synthetic construct
  REGION 1..108: note = JH1 VL
SYVLTQPPSVSVAPGKTARITCGGNNIGSKSVHWYQQKPGQAPVLVVYDDSDRPSGIPERFSGSNSGNTATLTISRVEAGDEADYYCQVWDSSSDHVVFGGGTKLTVL

SEQ ID NO: 3 - protein, 112 aa, synthetic construct
  REGION 1..112: note = JH2 VH
QVQLQESGPGLVKPSETLSLTCTVSGGSISSYYWSWIRQPPGKGLEWIGYINNSGSTNYNPSLKSRVTISVDTSKNQFSLKLSSVTAADTAVYYCAREDDYWGQGTLVTVSS

SEQ ID NO: 4 - protein, 109 aa, synthetic construct
  REGION 1..109: note = JH2 VL
EIVMTQSPATLSLSPGERATLSCRASQIISSSYLSWYQQKPGQAPRLLIYGASTRATGIPARFSGSGSGTDFTLTISSLQPEDFAVYYCQQDYDFPFTFGPGTKVDIKR

SEQ ID NO: 5 - protein, 115 aa, synthetic construct
  REGION 1..115: note = JH3 VH
QVQLQESGPGLVKPSETLSLTCTVSGGSISNYYWSWIRQPPGKGLEWIGYIYYSGSTIYNPSLKSRVTISVDTSKNQFSLKLSSVTAADTAVYYCAGDSSGIDYWGQGTLVTVSS

SEQ ID NO: 6 - protein, 108 aa, synthetic construct
  REGION 1..108: note = JH3 VL
EIVMTQSPATLSLSPGERATLSCRASQSVSSNYFSWYQQKPGQAPRLLIYGASTRATGIPARFSGSGSGTDFTLTISSLQPEDFAVYYCQQDYDFPFTFGPGTKVDIK

SEQ ID NO: 7 - protein, 112 aa, synthetic construct
  REGION 1..112: note = JH4 VH
QVQLQESGPGLVKPSETLSLTCTVSGGSISSYYWSWIRQSPGKGLEWIGYIHYSGNTNYNPSLKSRVTISVDTSKNQFSLKLSSVTAADTAVYYCAREDDYWGQGTLVTVSS

SEQ ID NO: 8 - protein, 108 aa, synthetic construct
  REGION 1..108: note = JH4 VL
EIVMTQSPATLSLSPGERATLSCRASQSVSSNYLSWYQQKPGQAPRLLIYGASTRATGIPARFSGSGSGTDFTLTISSLQPEDFAVYYCQQDFDFPFTFGPGTKVDIK

SEQ ID NO: 9 - protein, 120 aa, synthetic construct
  REGION 1..120: note = JH5 VH
EVQLLESGGGVAQPGRSLRLSCAASGFTFSSYGMHWVRQAPGKGLEWVAVISYDGSNKYYADSVKGRFTISRDNSKNTLYLQMNSLRAEDTAVYYCAKDRLRGSYGMDVWGQGTLVTVSS

SEQ ID NO: 10 - protein, 112 aa, synthetic construct
  REGION 1..112: note = JH5 VL
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGSWVFGGGTKLTVL

SEQ ID NO: 11 - protein, 240 aa, synthetic construct
  REGION 1..240: note = JH1 scFv
QVQLVQSGAEVKKPGASVKVSCKASGYTFTSYAMHWVRQAPGQRLEWMGWINAGNGNTKYSQKFQGRVTITRDTSASTAYMELRSLRSDDTAVYYCASGPGRLPVVWGQGTLVTVSSGGGGSGGGGSGGGGSSYVLTQPPSVSVAPGKTARITCGGNNIGSKSVHWYQQKPGQAPVLVVYDDSDRPSGIPERFSGSNSGNTATLTISRVEAGDEADYYCQVWDSSSDHVVFGGGTKLTVL

SEQ ID NO: 12 - protein, 235 aa, synthetic construct
  REGION 1..235: note = JH2 scFv
QVQLQESGPGLVKPSETLSLTCTVSGGSISSYYWSWIRQPPGKGLEWIGYINNSGSTNYNPSLKSRVTISVDTSKNQFSLKLSSVTAADTAVYYCAREDDYWGQGTLVTVSSGGGGSGGGGSGGGGSEIVMTQSPATLSLSPGERATLSCRASQIISSSYLSWYQQKPGQAPRLLIYGASTRATGIPARFSGSGSGTDFTLTISSLQPEDFAVYYCQQDYDFPFTFGPGTKVDIK

SEQ ID NO: 13 - protein, 238 aa, synthetic construct
  REGION 1..238: note = JH3 scFv
QVQLQESGPGLVKPSETLSLTCTVSGGSISNYYWSWIRQPPGKGLEWIGYIYYSGSTIYNPSLKSRVTISVDTSKNQFSLKLSSVTAADTAVYYCAGDSSGIDYWGQGTLVTVSSGGGGSGGGGSGGGGSEIVMTQSPATLSLSPGERATLSCRASQSVSSNYFSWYQQKPGQAPRLLIYGASTRATGIPARFSGSGSGTDFTLTISSLQPEDFAVYYCQQDYDFPFTFGPGTKVDIK

SEQ ID NO: 14 - protein, 235 aa, synthetic construct
  REGION 1..235: note = JH4 scFv
QVQLQESGPGLVKPSETLSLTCTVSGGSISSYYWSWIRQSPGKGLEWIGYIHYSGNTNYNPSLKSRVTISVDTSKNQFSLKLSSVTAADTAVYYCAREDDYWGQGTLVTVSSGGGGSGGGGSGGGGSEIVMTQSPATLSLSPGERATLSCRASQSVSSNYLSWYQQKPGQAPRLLIYGASTRATGIPARFSGSGSGTDFTLTISSLQPEDFAVYYCQQDFDFPFTFGPGTKVDIK

SEQ ID NO: 15 - protein, 247 aa, synthetic construct
  REGION 1..247: note = JH5 scFv
EVQLLESGGGVAQPGRSLRLSCAASGFTFSSYGMHWVRQAPGKGLEWVAVISYDGSNKYYADSVKGRFTISRDNSKNTLYLQMNSLRAEDTAVYYCAKDRLRGSYGMDVWGQGTLVTVSSGGGGSGGGGSGGGGSQSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGSWVFGGGTKLTVL

SEQ ID NO: 16 - protein, 7 aa, synthetic construct
  REGION 1..7: note = JH1 CDR-H1
GYTFTSY

SEQ ID NO: 17 - protein, 6 aa, synthetic construct
  REGION 1..6: note = JH1 CDR-H2
NAGNGN

SEQ ID NO: 18 - protein, 8 aa, synthetic construct
  REGION 1..8: note = JH1 CDR-H3
GPGRLPVV

SEQ ID NO: 19 - protein, 11 aa, synthetic construct
  REGION 1..11: note = JH1 CDR-L1
GGNNIGSKSVH

SEQ ID NO: 20 - protein, 7 aa, synthetic construct
  REGION 1..7: note = JH1 CDR-L2
DDSDRPS

SEQ ID NO: 21 - protein, 11 aa, synthetic construct
  REGION 1..11: note = JH1 CDR-L3
QVWDSSSDHVV

SEQ ID NO: 22 - protein, 7 aa, synthetic construct
  REGION 1..7: note = JH2 CDR-H1 JH4 CDR-H1
GGSISSY

SEQ ID NO: 23 - protein, 5 aa, synthetic construct
  REGION 1..5: note = JH2 CDR-H2
NNSGS

SEQ ID NO: 24 - protein, 4 aa, synthetic construct
  REGION 1..4: note = JH2 CDR-H3 JH4 CDR-H3
EDDY

SEQ ID NO: 25 - protein, 12 aa, synthetic construct
  REGION 1..12: note = JH2 CDR-L1
RASQIISSSYLS

SEQ ID NO: 26 - protein, 7 aa, synthetic construct
  REGION 1..7: note = JH2 CDR-L2 JH3 CDR-L2 JH4 CDR-L2
GASTRAT

SEQ ID NO: 27 - protein, 9 aa, synthetic construct
  REGION 1..9: note = JH2 CDR-L3 JH3 CDR-L3
QQDYDFPFT

SEQ ID NO: 28 - protein, 7 aa, synthetic construct
  REGION 1..7: note = JH3 CDR-H1
GGSISNY

SEQ ID NO: 29 - protein, 5 aa, synthetic construct
  REGION 1..5: note = JH3 CDR-H2
YYSGS

SEQ ID NO: 30 - protein, 7 aa, synthetic construct
  REGION 1..7: note = JH3 CDR-H3
DSSGIDY

SEQ ID NO: 31 - protein, 12 aa, synthetic construct
  REGION 1..12: note = JH3 CDR-L1
RASQSVSSNYFS

SEQ ID NO: 32 - protein, 5 aa, synthetic construct
  REGION 1..5: note = JH4 CDR-H2
HYSGN

SEQ ID NO: 33 - protein, 12 aa, synthetic construct
  REGION 1..12: note = JH4 CDR-L1
RASQSVSSNYLS

SEQ ID NO: 34 - protein, 9 aa, synthetic construct
  REGION 1..9: note = JH4 CDR-L3
QQDFDFPFT

SEQ ID NO: 35 - protein, 7 aa, synthetic construct
  REGION 1..7: note = JH5 CDR-H1
GFTFSSY

SEQ ID NO: 36 - protein, 6 aa, synthetic construct
  REGION 1..6: note = JH5 CDR-H2
SYDGSN

SEQ ID NO: 37 - protein, 11 aa, synthetic construct
  REGION 1..11: note = JH5 CDR-H3
DRLRGSYGMDV

SEQ ID NO: 38 - protein, 14 aa, synthetic construct
  REGION 1..14: note = JH5 CDR-L1
TGSSSNIGAGYDVH

SEQ ID NO: 39 - protein, 7 aa, synthetic construct
  REGION 1..7: note = JH5 CDR-L2
GNSNRPS

SEQ ID NO: 40 - protein, 12 aa, synthetic construct
  REGION 1..12: note = JH5 CDR-L3
QSYDSSLSGSWV

SEQ ID NO: 41 - protein, 120 aa, synthetic construct
  REGION 1..120: note = CD19 VH
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 42 - protein, 107 aa, synthetic construct
  REGION 1..107: note = CD19 VL
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 43 - protein, 5 aa, synthetic construct
  REGION 1..5: note = CD19 CDR-H1
DYGVS

SEQ ID NO: 44 - protein, 16 aa, synthetic construct
  REGION 1..16: note = CD19 CDR-H2
VIWGSETTYYNSALKS

SEQ ID NO: 45 - protein, 7 aa, synthetic construct
  REGION 1..7: note = CD19 CDR-H3
YAMDYWG

SEQ ID NO: 46 - protein, 12 aa, synthetic construct
  REGION 1..12: note = CD19 CDR-H3
HYYYGGSYAMDY

SEQ ID NO: 47 - protein, 11 aa, synthetic construct
  REGION 1..11: note = CD19 CDR-L1
RASQDISKYLN

SEQ ID NO: 48 - protein, 7 aa, synthetic construct
  REGION 1..7: note = CD19 CDR-L2
SRLHSGV

SEQ ID NO: 49 - protein, 7 aa, synthetic construct
  REGION 1..7: note = CD19 CDR-L2
HTSRLHS

SEQ ID NO: 50 - protein, 9 aa, synthetic construct
  REGION 1..9: note = CD19 CDR-L3
GNTLPYTFG

SEQ ID NO: 51 - protein, 9 aa, synthetic construct
  REGION 1..9: note = CD19 CDR-L3
QQGNTLPYT

SEQ ID NO: 52 - protein, 13 aa, synthetic construct
  REGION 1..13: note = Short Spacer (IgG4 hinge) (aa)
ESKYGPPCPPCPM

SEQ ID NO: 53 - protein, 228 aa, synthetic construct
  REGION 1..228: note = Long spacer (IgG4/IgG2 hinge- IgG2/IgG4 CH2- IgG4 CH3 spacer
ESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 54 - DNA, 683 bp, synthetic construct
  misc_feature 1..683: note = IgG4/IgG2 hinge- IgG2/IgG4 CH2- IgG4 CH3 spacer (nt)
  source 1..683: mol_type = other DNA
gaatctaagtacggaccgccctgccctccctgccctgctcctcctgtggctggaccaagcgtgttcctgtttccacctaagcctaaagataccctgatgatttcccgcacacctgaagtgacttgcgtggtcgtggacgtgagccaggaggatccagaagtgcagttcaactggtacgtggacggcgtggaagtccacaatgctaagactaaaccccgagaggaacagtttcagtcaacttaccgggtcgtgagcgtgctgaccgtcctgcatcaggattggctgaacgggaaggagtataagtgcaaagtgtctaataagggactgcctagctccatcgagaaaacaattagtaaggcaaaagggcagcctcgagaaccacaggtgtataccctgccccctagccaggaggaaatgaccaagaaccaggtgtccctgacatgtctggtcaaaggcttctatccaagtacatcgccgtggagtgggaatcaaatgggcagcccgagaacaattacaagaccacaccacccgtgctggactctgatggaagtttctttctgtattccaggctgaccgtggataaatctcgctggcaggagggcaacgtgttctcttgcagtgtcatgcacgaagccctgcacaatcattatacacagaagtcactgagcctgtccctgggcaaa

SEQ ID NO: 55 - protein, 27 aa, synthetic construct
  REGION 1..27: note = CD28 transmembrane domain (aa)
FWVLVVVGGVLACYSLLVTVAFIIFWV

SEQ ID NO: 56 - protein, 42 aa, synthetic construct
  REGION 1..42: note = 4-1BB-derived intracellular co-signaling sequence (aa)
KRGRKKLLYIFKQPFMRPVQTTQEEDGCSCRFPEEEEGGCEL

SEQ ID NO: 57 - protein, 112 aa, synthetic construct
  REGION 1..112: note = CD3-zeta derived intracellular signaling domain (aa)
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 58 - protein, 15 aa, synthetic construct
  REGION 1..15: note = Linker
GGGGSGGGGSGGGGS

SEQ ID NO: 59 - protein, 18 aa, synthetic construct
  REGION 1..18: note = Linker
GSTSGSGKPGSGEGSTKG

SEQ ID NO: 60 - protein, 5 aa, synthetic construct
  REGION 1..5: note = Linker
GGGGS

SEQ ID NO: 61 - protein, 10 aa, synthetic construct
  REGION 1..10: note = Linker
GGGGSGGGGS

SEQ ID NO: 62 - protein, 20 aa, synthetic construct
  REGION 1..20: note = Linker
GGGGSGGGGSGGGGSGGGGS

SEQ ID NO: 63 - protein, 125 aa, synthetic construct
  REGION 1..125: note = CD19 clone 5 VH; CD19 clone 17 VH
EVQLVESGGGLVQPGRSLRLSCAASGFTFDDYAMHWVRQAPGKGLEWVSGISWNSGRIGYADSVKGRFTISRDNAKNSLFLQMNSLRAEDTAVYYCARDQGYHYYDSAEHAFDIWGQGTMVTVSS

SEQ ID NO: 64 - protein, 125 aa, synthetic construct
  REGION 1..125: note = CD19 clone 18 VH; CD19 clone 18B VH; CD19 clone 76 VH
EVQLVESGGGLVQPGRSLRLSCAASGFTFDDYAMHWVRQAPGKGLEWVSGISWNSGRIGYADSVKGRFTISRDNAKNSLFLQMNSLRAEDTAVYYCARDQGYHYYDSAEHAFDIWGQGTVVTVSS

SEQ ID NO: 65 - protein, 108 aa, synthetic construct
  REGION 1..108: note = CD19 clone 5 VL
SYELTQDPAVSVALGQTVRITCQGDSLRSYYASWYQQKPGQAPVLVIYDKNNRPSGIPDRFSGSSSGNTASLTITGAQAEDEADYYCNSRDSSGNNWVFGGGTKLTVL

SEQ ID NO: 66 - protein, 110 aa, synthetic construct
  REGION 1..110: note = CD19 clone 17 VL
QSALTQPASVSGSPGQSITIFCTGTSSDVGGYNYVSWYQQLPGTAPKLLIYSNNQRPSGVPDRFSGSKSGTSASLAISGLRSEDEADYYCAAWDDSLSVVFGGGTKLTVL

SEQ ID NO: 67 - protein, 106 aa, synthetic construct
  REGION 1..106: note = CD19 clone 18 VL
QSALTQPRSVSGFPGQSVTISCTGTTSDDVSWYQQHPGKAPQLMLYDVSKRPSGVPHRFSGSRSGRAASLIISGLQTEDEADYFCCSYAGRYNSVLFGGGTKLTVL

SEQ ID NO: 68 - protein, 106 aa, synthetic construct
  REGION 1..106: note = CD19 clone 18B VL
QSALTQPRSVSGFPGQSVTISCTGTTSDDVSWYQQHPGKAPQLMLYDVSKRPSGVPHRFSGSRSGRAASLIISGLQTEDEADYFCSSYAGRYNSVLFGGGTKLTVL

SEQ ID NO: 69 - protein, 109 aa, synthetic construct
  REGION 1..109: note = CD19 clone 76 VL
QSVLTQPPSVSAAPGQEVTISCSGSSSNIGNNYVSWYQQLPGTAPKLLIYDNDKRPSGIPDRFSGSKSGTSATLGITGLQTGDEADYYCGTWDGNLSAVFGGGTKVTVL

SEQ ID NO: 70 - protein, 248 aa, synthetic construct
  REGION 1..248: note = CD19 clone 5 scFv
EVQLVESGGGLVQPGRSLRLSCAASGFTFDDYAMHWVRQAPGKGLEWVSGISWNSGRIGYADSVKGRFTISRDNAKNSLFLQMNSLRAEDTAVYYCARDQGYHYYDSAEHAFDIWGQGTMVTVSSGGGGSGGGGSGGGGSSYELTQDPAVSVALGQTVRITCQGDSLRSYYASWYQQKPGQAPVLVIYDKNNRPSGIPDRFSGSSSGNTASLTITGAQAEDEADYYCNSRDSSGNNWVFGGGTKLTVL

SEQ ID NO: 71 - protein, 250 aa, synthetic construct
  REGION 1..250: note = CD19 clone 17 scFv
EVQLVESGGGLVQPGRSLRLSCAASGFTFDDYAMHWVRQAPGKGLEWVSGISWNSGRIGYADSVKGRFTISRDNAKNSLFLQMNSLRAEDTAVYYCARDQGYHYYDSAEHAFDIWGQGTMVTVSSGGGGSGGGGSGGGGSQSALTQPASVSGSPGQSITIFCTGTSSDVGGYNYVSWYQQLPGTAPKLLIYSNNQRPSGVPDRFSGSKSGTSASLAISGLRSEDEADYYCAAWDDSLSVVFGGGTKLTVL

SEQ ID NO: 72 - protein, 246 aa, synthetic construct
  REGION 1..246: note = CD19 clone 18 scFv
EVQLVESGGGLVQPGRSLRLSCAASGFTFDDYAMHWVRQAPGKGLEWVSGISWNSGRIGYADSVKGRFTISRDNAKNSLFLQMNSLRAEDTAVYYCARDQGYHYYDSAEHAFDIWGQGTVVTVSSGGGGSGGGGSGGGGSQSALTQPRSVSGFPGQSVTISCTGTTSDDVSWYQQHPGKAPQLMLYDVSKRPSGVPHRFSGSRSGRAASLIISGLQTEDEADYFCCSYAGRYNSVLFGGGTKLTVL

SEQ ID NO: 73 - protein, 246 aa, synthetic construct
  REGION 1..246: note = CD19 clone 18B scFv
EVQLVESGGGLVQPGRSLRLSCAASGFTFDDYAMHWVRQAPGKGLEWVSGISWNSGRIGYADSVKGRFTISRDNAKNSLFLQMNSLRAEDTAVYYCARDQGYHYYDSAEHAFDIWGQGTVVTVSSGGGGSGGGGSGGGGSQSALTQPRSVSGFPGQSVTISCTGTTSDDVSWYQQHPGKAPQLMLYDVSKRPSGVPHRFSGSRSGRAASLIISGLQTEDEADYFCSSYAGRYNSVLFGGGTKLTVL

SEQ ID NO: 74 - protein, 249 aa, synthetic construct
  REGION 1..249: note = CD19 clone 76 scFv
EVQLVESGGGLVQPGRSLRLSCAASGFTFDDYAMHWVRQAPGKGLEWVSGISWNSGRIGYADSVKGRFTISRDNAKNSLFLQMNSLRAEDTAVYYCARDQGYHYYDSAEHAFDIWGQGTVVTVSSGGGGSGGGGSGGGGSQSVLTQPPSVSAAPGQEVTISCSGSSSNIGNNYVSWYQQLPGTAPKLLIYDNDKRPSGIPDRFSGSKSGTSATLGITGLQTGDEADYYCGTWDGNLSAVFGGGTKVTVL

SEQ ID NO: 75 - protein, 5 aa, synthetic construct
  REGION 1..5: note = CD19 clone 5, 17, 18, 18B, 76 VH CDR1
DYAMH

SEQ ID NO: 76 - protein, 17 aa, synthetic construct
  REGION 1..17: note = CD19 clone 5, 17, 18, 18B, 76 VH CDR2
GISWNSGRIGYADSVKG

SEQ ID NO: 77 - protein, 16 aa, synthetic construct
  REGION 1..16: note = CD19 clone 5, 17, 18, 18B, 76 VH CDR3
DQGYHYYDSAEHAFDI

SEQ ID NO: 78 - protein, 11 aa, synthetic construct
  REGION 1..11: note = CD19 clone 5 VL CDR1
QGDSLRSYYAS

SEQ ID NO: 79 - protein, 7 aa, synthetic construct
  REGION 1..7: note = CD19 clone 5 VL CDR2
DKNNRPS

SEQ ID NO: 80 - protein, 11 aa, synthetic construct
  REGION 1..11: note = CD19 clone 5 VL CDR3
NSRDSSGNNWV

SEQ ID NO: 81 - protein, 14 aa, synthetic construct
  REGION 1..14: note = CD19 clone 17 VL CDR1
TGTSSDVGGYNYVS

SEQ ID NO: 82 - protein, 7 aa, synthetic construct
  REGION 1..7: note = CD19 clone 17 VL CDR2
SNNQRPS

SEQ ID NO: 83 - protein, 10 aa, synthetic construct
  REGION 1..10: note = CD19 clone 17 VL CDR3
AAWDDSLSVV

SEQ ID NO: 84 - protein, 9 aa, synthetic construct
  REGION 1..9: note = CD19 clone 18 VL CDR1; CD19 clone 18B VL CDR1
TGTTSDDVS

SEQ ID NO: 85 - protein, 7 aa, synthetic construct
  REGION 1..7: note = CD19 clone 18 VL CDR2; CD19 clone 18B VL CDR2
DVSKRPS

SEQ ID NO: 86 - protein, 11 aa, synthetic construct
  REGION 1..11: note = CD19 clone 18 VL CDR3
CSYAGRYNSVL

SEQ ID NO: 87 - protein, 11 aa, synthetic construct
  REGION 1..11: note = CD19 clone 18B VL CDR3
SSYAGRYNSVL

SEQ ID NO: 88 - protein, 13 aa, synthetic construct
  REGION 1..13: note = CD19 clone 76 VL CDR1
SGSSSNIGNNYVS

SEQ ID NO: 89 - protein, 7 aa, synthetic construct
  REGION 1..7: note = CD19 clone 76 VL CDR2
DNDKRPS

SEQ ID NO: 90 - protein, 10 aa, synthetic construct
  REGION 1..10: note = CD19 clone 76 VL CDR3
GTWDGNLSAV

SEQ ID NO: 91 - skipped

SEQ ID NO: 92 - protein, 326 aa, synthetic construct
  REGION 1..326: note = Human IgG2 Fc (Uniprot P01859)
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 93 - protein, 327 aa, synthetic construct
  REGION 1..327: note = Human IgG4 Fc (Uniprot P01861)
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 94 - protein, 671 aa, synthetic construct
  REGION 1..671: note = JH1 CAR/LoP
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSQVQLVQSGAEVKKPGASVKVSCKASGYTFTSYAMHWVRQAPGQRLEWMGWINAGNGNTKYSQKFQGRVTITRDTSASTAYMELRSLRSDDTAVYYCASGPGRLPVVWGQGTLVTVSSGGGGSGGGGSGGGGSSYVLTQPPSVSVAPGKTARITCGGNNIGSKSVHWYQQKPGQAPVLVVYDDSDRPSGIPERFSGSNSGNTATLTISRVEAGDEADYYCQVWDSSSDHVVFGGGTKLTVLGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 95 - protein, 680 aa, synthetic construct
  REGION 1..680: note = JH2 CAR/LiD/a
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSQVQLQESGPGLVKPSETLSLTCTVSGGSISSYYWSWIRQPPGKGLEWIGYINNSGSTNYNPSLKSRVTISVDTSKNQFSLKLSSVTAADTAVYYCAREDDYWGQGTLVTVSSGGGGSGGGGSGGGGSEIVMTQSPATLSLSPGERATLSCRASQIISSSYLSWYQQKPGQAPRLLIYGASTRATGIPARFSGSGSGTDFTLTISSLQPEDFAVYYCQQDYDFPFTFGPGTKVDIKRESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 96 - protein, 667 aa, synthetic construct
  REGION 1..667: note = JH2 CAR/LoP
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSQVQLQESGPGLVKPSETLSLTCTVSGGSISSYYWSWIRQPPGKGLEWIGYINNSGSTNYNPSLKSRVTISVDTSKNQFSLKLSSVTAADTAVYYCAREDDYWGQGTLVTVSSGGGGSGGGGSGGGGSEIVMTQSPATLSLSPGERATLSCRASQIISSSYLSWYQQKPGQAPRLLIYGASTRATGIPARFSGSGSGTDFTLTISSLQPEDFAVYYCQQDYDFPFTFGPGTKVDIKRGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 97 - protein, 680 aa, synthetic construct
  REGION 1..680: note = JH2 CAR/LiD/b
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSEIVMTQSPATLSLSPGERATLSCRASQIISSSYLSWYQQKPGQAPRLLIYGASTRATGIPARFSGSGSGTDFTLTISSLQPEDFAVYYCQQDYDFPFTFGPGTKVDIKRGGGGSGGGGSGGGGSQVQLQESGPGLVKPSETLSLTCTVSGGSISSYYWSWIRQPPGKGLEWIGYINNSGSTNYNPSLKSRVTISVDTSKNQFSLKLSSVTAADTAVYYCAREDDYWGQGTLVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 98 - protein, 680 aa, synthetic construct
  REGION 1..680: note = JH2 CAR/LoD/a
EIVMTQSPATLSLSPGERATLSCRASQIISSSYLSWYQQKPGQAPRLLIYGASTRATGIPARFSGSGSGTDFTLTISSLQPEDFAVYYCQQDYDFPFTFGPGTKVDIKRGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSQVQLQESGPGLVKPSETLSLTCTVSGGSISSYYWSWIRQPPGKGLEWIGYINNSGSTNYNPSLKSRVTISVDTSKNQFSLKLSSVTAADTAVYYCAREDDYWGQGTLVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 99 - protein, 670 aa, synthetic construct
  REGION 1..670: note = JH2 CAR/LoD/b
QVQLQESGPGLVKPSETLSLTCTVSGGSISSYYWSWIRQPPGKGLEWIGYINNSGSTNYNPSLKSRVTISVDTSKNQFSLKLSSVTAADTAVYYCAREDDYWGQGTLVTVSSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSEIVMTQSPATLSLSPGERATLSCRASQIISSSYLSWYQQKPGQAPRLLIYGASTRATGIPARFSGSGSGTDFTLTISSLQPEDFAVYYCQQDYDFPFTFGPGTKVDIKRESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 100 - protein, 683 aa, synthetic construct
  REGION 1..683: note = JH3 CAR/LoD
EIVMTQSPATLSLSPGERATLSCRASQSVSSNYFSWYQQKPGQAPRLLIYGASTRATGIPARFSGSGSGTDFTLTISSLQPEDFAVYYCQQDYDFPFTFGPGTKVDIKRGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSQVQLQESGPGLVKPSETLSLTCTVSGGSISNYYWSWIRQPPGKGLEWIGYIYYSGSTIYNPSLKSRVTISVDTSKNQFSLKLSSVTAADTAVYYCAGDSSGIDYWGQGTLVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 101 - protein, 680 aa, synthetic construct
  REGION 1..680: note = JH4 CAR/LiD
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSQVQLQESGPGLVKPSETLSLTCTVSGGSISSYYWSWIRQSPGKGLEWIGYIHYSGNTNYNPSLKSRVTISVDTSKNQFSLKLSSVTAADTAVYYCAREDDYWGQGTLVTVSSGGGGSGGGGSGGGGSEIVMTQSPATLSLSPGERATLSCRASQSVSSNYLSWYQQKPGQAPRLLIYGASTRATGIPARFSGSGSGTDFTLTISSLQPEDFAVYYCQQDFDFPFTFGPGTKVDIKRESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 102 - protein, 700 aa, synthetic construct
  REGION 1..700: note = JH2 CAR/LiD/c
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSGGGGSGGGGSEIVMTQSPATLSLSPGERATLSCRASQIISSSYLSWYQQKPGQAPRLLIYGASTRATGIPARFSGSGSGTDFTLTISSLQPEDFAVYYCQQDYDFPFTFGPGTKVDIKRGGGGSGGGGSGGGGSGGGGSQVQLQESGPGLVKPSETLSLTCTVSGGSISSYYWSWIRQPPGKGLEWIGYINNSGSTNYNPSLKSRVTISVDTSKNQFSLKLSSVTAADTAVYYCAREDDYWGQGTLVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 103 - protein, 678 aa, synthetic construct
  REGION 1..678: note = JH5 CAR/LoP
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSEVQLLESGGGVAQPGRSLRLSCAASGFTFSSYGMHWVRQAPGKGLEWVAVISYDGSNKYYADSVKGRFTISRDNSKNTLYLQMNSLRAEDTAVYYCAKDRLRGSYGMDVWGQGTLVTVSSGGGGSGGGGSGGGGSQSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGSWVFGGGTKLTVLGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 104 - protein, 16 aa, synthetic construct
  REGION 1..16: note = CD33 signal peptide
MPLLLLLPLLWAGALA

SEQ ID NO: 105 - DNA, 544 bp, synthetic construct
  misc_feature 1..544: note = EF1alpha promoter with HTLV1 enhancer
  source 1..544: mol_type = other DNA
ggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctac

SEQ ID NO: 106 - DNA, 589 bp, synthetic construct
  misc_feature 1..589: note = Woodchuck Hepatitis Virus (WHP) Posttranscriptional Regulatory Element (WPRE)
  source 1..589: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 107 - protein, 22 aa, synthetic construct
  REGION 1..22: note = F2A peptide
VKQTLNFDLLKLAGDVESNPGP

SEQ ID NO: 108 - protein, 25 aa, synthetic construct
  REGION 1..25: note = F2A peptide
GSGVKQTLNFDLLKLAGDVESNPGP

SEQ ID NO: 109 - protein, 20 aa, synthetic construct
  REGION 1..20: note = E2A peptide
QCTNYALLKLAGDVESNPGP

SEQ ID NO: 110 - protein, 23 aa, synthetic construct
  REGION 1..23: note = E2A peptide
GSGQCTNYALLKLAGDVESNPGP

SEQ ID NO: 111 - protein, 24 aa, synthetic construct
  REGION 1..24: note = T2A peptide
LEGGGEGRGSLLTCGDVEENPGPR

SEQ ID NO: 112 - protein, 18 aa, synthetic construct
  REGION 1..18: note = T2A peptide
EGRGSLLTCGDVEENPGP

SEQ ID NO: 113 - protein, 21 aa, synthetic construct
  REGION 1..21: note = T2A peptide
GSGEGRGSLLTCGDVEENPGP

SEQ ID NO: 114 - protein, 22 aa, synthetic construct
  REGION 1..22: note = P2A peptide
GSGATNFSLLKQAGDVEENPGP

SEQ ID NO: 115 - protein, 19 aa, synthetic construct
  REGION 1..19: note = P2A peptide
ATNFSLLKQAGDVEENPGP

SEQ ID NO: 116 - protein, 357 aa, synthetic construct
  REGION 1..357: note = tEGFR
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 117 - protein, 335 aa, synthetic construct
  REGION 1..335: note = tEGFR
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 118 - protein, 357 aa, synthetic construct; identical to SEQ ID NO: 116
  REGION 1..357: note = Truncated EGFR (tEGFR)

SEQ ID NO: 119 - protein, 245 aa, synthetic construct
  REGION 1..245: note = CD19 scFv
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 120 - protein, 184 aa, synthetic construct
  REGION 1..184: note = Human BAFF-R
MRRGPRSLRGRDAPAPTPCVPAECFDLLVRHCVACGLLRTPRPKPAGASSPAPRTALQPQESVGAGAGEAALPLPGLLFGAPALLGLALVLALVLVGLVSWRRRQRRLRGASSAEAPDGDKDAPEPLDKVIILSPGISDATAPAWPPPGEDPGTTPPGHSVPVPATELGSTELVTTKTAGPEQQ

SEQ ID NO: 121 - protein, 11 aa, synthetic construct
  REGION 1..11: note = CDR L1
KASQNVGTNVA

SEQ ID NO: 122 - protein, 7 aa, synthetic construct
  REGION 1..7: note = CDR L2
SATYRNS

SEQ ID NO: 123 - protein, 9 aa, synthetic construct
  REGION 1..9: note = CDR L3
QQYNRYPYT

SEQ ID NO: 124 - protein, 5 aa, synthetic construct
  REGION 1..5: note = CDR H1
SYWMN

SEQ ID NO: 125 - protein, 17 aa, synthetic construct
  REGION 1..17: note = CDR H2
QIYPGDGDTNYNGKFKG

SEQ ID NO: 126 - protein, 13 aa, synthetic construct
  REGION 1..13: note = CDR H3
KTISSVVDFYFDY

SEQ ID NO: 127 - protein, 122 aa, synthetic construct
  REGION 1..122: note = VH
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSS

SEQ ID NO: 128 - protein, 108 aa, synthetic construct
  REGION 1..108: note = VL
DIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKR

SEQ ID NO: 129 - DNA, 4008 bp, synthetic construct
  source 1..4008: mol_type = other DNA
  misc_feature 1..4008: note = JH2 CAR/LoD/b (nucleic acid)
ctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgatggatccggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacggctagcgccaccatgccgctgctgctactgctgcccctgctgtgggcaggggctctagctcaagtgcagctgcaggagtcgggccctggcctggtgaaaccgtctgagaccctcagcttgacctgcactgtctccggcgggtccatctctagctattattggtcatggatccggcagcccccaggcaaaggactcgagtggatcggctacattaacaactccgggagcaccaattacaacccttcgctaaagtctcgtgtgaccatctcagtcgacacttccaagaaccagttttcgttaaagctgagctctgtcaccgctgcggacaccgccgtgtactattgtgcccgtgaggatgattactggggacagggaaccctggttacagtatcgtccggcggaggtggttcggacatccagatgacccagaccacgtcctccttgtcggcttccctgggggaccgcgtgacgatctcatgccgagcaagccaggacatttctaagtacctcaattggtaccagcagaagcccgacggcaccgtcaagctgctgatttaccatacctcccgacttcactctggggtgccctctcgcttctccggctctgggtcaggcaccgactactccctcactatatccaacctggagcaggaagatatcgccacctacttctgtcagcagggcaacacgctgccgtacaccttcggcggcggcacaaaactggagatcaccggaagcaccagtgggtccggcaagccaggctctggggagggttcgaccaagggcgaggtgaagctgcaggagtccggccccggacttgtggctcctagccagtctctatctgtgacgtgcacagtttcgggagtgagcctgcccgactacggcgtcagctggatccgccagccaccgcgcaagggactggagtggctgggtgtgatctggggctcggagaccacctactacaattccgcgctgaagtcccgcctcactatcatcaaggacaactctaaaagccaggtgttcctgaagatgaacagtttgcagactgatgacactgccatctactactgtgccaagcactactactacggcggttcctatgccatggattattggggccagggcacatcggtgaccgtgtccagtggaggtggcggcagcgagatcgtcatgacccagagccctgcgaccctttcgctctccccgggcgagcgcgctactctctcttgccgcgcgagtcaaattatctcctcaagctacctgagctggtaccagcagaagcctggtcaggcccctagactgctgatctatggcgcatccacccgcgcgaccggcattcccgctcgtttttccggctctggatccggaacggacttcacactgactataagctctcttcagccggaagatttcgccgtatactactgtcaacaggactacgactttcccttcaccttcggccccggcaccaaggtggacatcaaacgtgagtctaaatacggaccgccttgtcctccttgtcccatgttctgggtgctcgtggtcgttggcggagtgctggcctgttactccctgctggttaccgtggccttcatcatcttttgggtcaagcggggcagaaagaagctgctctacatcttcaagcagcccttcatgcggcccgtgcagaccacacaagaggaagatggctgctcctgccgattccccgaggaagaagaaggcggctgcgagctgagagtgaagttcagcagatccgccgacgctccagcctatcagcagggccaaaaccagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctagatgagtcgactgctttatttgtgaaatttgtgatgctattgctttatttgtaaccattataagctgcaataaacaagttaacaacaacaattgcattcattttatgtttcaggttcagggggaggtgtgggaggttttttaaagggccctttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtg

SEQ ID NO: 130 - protein, 686 aa, synthetic construct
  REGION 1..686: note = JH2 CAR/LoD/b (amino acid sequence)
MPLLLLLPLLWAGALAQVQLQESGPGLVKPSETLSLTCTVSGGSISSYYWSWIRQPPGKGLEWIGYINNSGSTNYNPSLKSRVTISVDTSKNQFSLKLSSVTAADTAVYYCAREDDYWGQGTLVTVSSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSEIVMTQSPATLSLSPGERATLSCRASQIISSSYLSWYQQKPGQAPRLLIYGASTRATGIPARFSGSGSGTDFTLTISSLQPEDFAVYYCQQDYDFPFTFGPGTKVDIKRESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

(19) United States
(12) Patent Application Publication
(10) Pub. No.: US 2026/0207667 A1
(43) Pub. Date: Jul. 23, 2026

(54) CHIMERIC ANTIGEN RECEPTORS SPECIFIC FOR BAFF-R AND CD19 AND METHODS AND USES THEREOF

(71) Applicant: Juno Therapeutics, Inc., Seattle, WA (US)

(72) Inventors: Ruth Amanda SALMON, Seattle, WA (US); Douglas JONES, Seattle, WA (US); Brian Joshua BELMONT, Seattle, WA (US); Madeline D. WILLIAMS, Seattle, WA (US); Rebekah TURK, Seattle, WA (US); Eric William JEFFERY, Seattle, WA (US); Andres ALVAREZ, Seattle, WA (US); Yue JIANG, Seattle, WA (US); Gabriela DIAZ, Seattle, WA (US); Thomas COX, Redmond, WA (US); Chia-Yung WU, Seattle, WA (US); Tiffany HUELAR, Seattle, WA (US); Jon Christopher JONES, Seattle, WA (US); Stephanie BUSCH, Seattle, WA (US); Cédric CLEYRAT, Seattle, WA (US)

(73) Assignee: Juno Therapeutics, Inc., Seattle, WA (US)

(21) Appl. No.: 19/138,595

(22) PCT Filed: Dec. 12, 2023
(86) PCT No.: PCT/US2023/083693

§ 371 Date: Jun. 12, 2025

Related U.S. Application Data
(60) Provisional application No. 63/432,342, filed on Dec. 13, 2022.

(51) Int. Cl. A61K 35/17; A61K 40/11; A61K 40/42; A61P 35/02; C07K 14/705; C07K 14/725; C07K 16/28; C12N 15/86

(52) U.S. Cl. CPC A61K 35/17; A61K 40/11; A61K 40/4211; A61K 40/4214; A61P 35/02; C07K 14/7051; C07K 14/70521; C07K 14/70578; C07K 16/2803; C07K 16/2878; C12N 15/86; A61K 2239/13; A61K 2239/17; A61K 2239/21; A61K 2239/22; A61K 2239/29; A61K 2239/38; A61K 2239/48; C07K 2317/524; C07K 2317/526; C07K 2317/565; C12N 2740/15043; C12N 2800/22

(57) ABSTRACT

Provided are B cell-activating factor receptor (BAFF-R)-binding molecules, in particular, to human antibodies specific for BAFF-R, including antibody fragments. The present disclosure further relates to recombinant receptors, including chimeric antigen receptors (CARs) that contain such antibodies or fragments, and polynucleotides that encode the antibodies, antigen-binding fragments or receptors specific for BAFF-R. Also provided are CARs which contain extracellular binding domains that bind to BAFF-R and B-lymphocyte antigen CD19 (CD19), genetically engineered cells expressing such CARs, and uses thereof in adoptive cell therapy.

### CROSS-REFERENCE TO RELATED APPLICATIONS

This application claims priority from U.S. provisional application No. 63/432,342 filed Dec. 13, 2022, entitled “Chimeric Antigen Receptors Specific for BAFF-R and CD19 and Methods and Uses Thereof,” the contents of which is incorporated by reference in its entirety.

### INCORPORATION BY REFERENCE OF SEQUENCE LISTING

The present application is being filed along with a Sequence Listing in electronic format. The Sequence Listing is provided as a file entitled 735042023340SeqList.xml, created Dec. 11, 2023, which is 181,862 bytes in size. The information in the electronic format of the Sequence Listing is herein incorporated by reference in its entirety.

### FIELD

The present disclosure relates in some aspects to chimeric antigen receptors (CARs), which contain extracellular binding domains that bind to B cell-activating factor receptor (BAFF-R) and B-lymphocyte antigen CD19 (CD19). The present disclosure further relates to BAFF-R-binding molecules, in particular, to anti-BAFF-R antibodies including antibody fragments. The disclosure also provides genetically engineered cells such as T cells containing the provided CARs, and related methods and uses thereof in adoptive cell therapy.

### BACKGROUND

CAR-T cell therapies have shown to be effective in treating cancers. However, due to the heterogeneous character of tumor cells, CAR-T therapies focused on the targeting of a single antigen may be insufficient. Additionally, tumor cells can down-regulate or mutate the CAR-T target antigen expressed on the cell surface in order to escape detection. Therefore, there is a need to produce a CAR-T cell which expresses two different targets simultaneously. Provided are embodiments that meet such needs.

### SUMMARY

Provided herein is a bispecific chimeric antigen receptor (CAR) comprising an extracellular binding domain, a spacer, a transmembrane domain, and an intracellular signaling domain, wherein the extracellular binding domain comprises: a B-cell activating factor receptor (BAFF-R)-binding domain that binds to BAFF-R comprising a heavy chain variable (V_{H}) region and a light chain variable (V_{L}) region; and a CD19-binding domain that binds to CD19 comprising a V_{H} region and a V_{L} region, wherein the extracellular binding domain comprises in order from the amino- to carboxy-terminus: (i) the V_{H} region of the BAFF-R-binding domain, the V_{L} region of the CD19-binding domain, the V_{H} region of the CD19-binding domain, and the V_{L} region of the BAFF-R binding domain; (ii) the V_{L} region of the BAFF-R-binding domain, the V_{L} region of the CD19-binding domain, the V_{H} region of the CD19-binding domain, and the V_{H} region of the BAFF-R binding domain; (iii) the V_{H} region of the BAFF-R-binding domain, the V_{H} region of the CD19-binding domain, the V_{L} region of the CD19-binding domain, and the V_{L} region of the BAFF-R-binding domain; (iv) the V_{L} region of the BAFF-R-binding domain, the V_{H} region of the CD19-binding domain, the V_{L} region of the CD19-binding domain, and the V_{H} region of the BAFF-R binding domain; (v) the V_{L} region of the CD19-binding domain, the V_{H} region of the BAFF-R-binding domain, the V_{L} region of the BAFF-R-binding domain, and the V_{H} region of the CD19-binding domain; (vi) the V_{H} region of the CD19-binding domain, the V_{H} region of the BAFF-R-binding domain, the V_{L} region of the BAFF-R-binding domain, and the V_{L} region of the CD19-binding domain; (vii) the V_{L} region of the CD19-binding domain, the V_{H} region of the CD19-binding domain, the V_{H} region of the BAFF-R-binding domain, and the V_{L} region of the BAFF-R-binding domain; (viii) the V_{L} region of the CD19-binding domain, the V_{H} region of the CD19-binding domain, the V_{L} region of the BAFF-R-binding domain, and the V_{H} region of the BAFF-R-binding domain; (ix) the V_{H} region of the CD19-binding domain, the V_{L} region of the CD19-binding domain, the V_{H} region of the BAFF-R-binding domain, and the V_{L} region of the BAFF-R-binding domain; (x) the V_{H} region of the CD19-binding domain, the V_{L} region of the CD19-binding domain, the V_{L} region of the BAFF-R-binding domain, and the V_{H} region of the BAFF-R-binding domain; (xi) the V_{L} region of the BAFF-R-binding domain, the V_{H} region of the BAFF-R-binding domain, the V_{H} region of the CD19-binding domain, and the V_{L} region of the CD19-binding domain; (xii) the V_{L} region of the BAFF-R-binding domain, the V_{H} region of the BAFF-R-binding domain, the V_{L} region of the CD19-binding domain, and the V_{H} region of the CD19-binding domain; (xiii) the V_{H} region of the BAFF-R-binding domain, the V_{L} region of the BAFF-R-binding domain, the V_{H} region of the CD19-binding domain, and the V_{L} region of the CD19-binding domain; (xiv) the V_{H} region of the BAFF-R-binding domain, the V_{L} region of the BAFF-R-binding domain, the V_{L} region of the CD19-binding domain, and the V_{H} region of the CD19-binding domain; (xv) the V_{L} region of the CD19-binding domain, the V_{L} region of the BAFF-R-binding domain, the V_{H} region of the BAFF-R-binding domain, and the V_{H} region of the CD19-binding domain; or (xvi) the V_{H} region of the CD19-binding domain, the V_{L} region of the BAFF-R-binding domain, the V_{H} region of the BAFF-R-binding domain, and the V_{L} region of the CD19-binding domain.

Provided herein is a bispecific chimeric antigen receptor (CAR) comprising an extracellular binding domain, a spacer, a transmembrane domain, and an intracellular signaling domain, wherein the extracellular binding domain comprises: a B-cell activating factor receptor (BAFF-R)-binding domain that binds to BAFF-R comprising a heavy chain variable (V_{H}) region and a light chain variable (V_{L}) region; and a CD19-binding domain that binds to CD19 comprising a V_{H} region and a V_{L} region, wherein the extracellular binding domain comprises in order from the amino- to carboxy-terminus: (i) the V_{H} region of the BAFF-R-binding domain, the V_{L} region of the CD19-binding domain, the V_{H} region of the CD19-binding domain, and the V_{L} region of the BAFF-R binding domain; (ii) the V_{L} region of the BAFF-R-binding domain, the V_{L} region of the CD19-binding domain, the V_{H} region of the CD19-binding domain, and the V_{H} region of the BAFF-R binding domain; (iii) the V_{H} region of the BAFF-R-binding domain, the V_{H} region of the CD19-binding domain, the V_{L} region of the CD19-binding domain, and the V_{L} region of the BAFF-R binding domain; or (iv) the V_{L} region of the BAFF-R-binding domain, the V_{H} region of the CD19-binding domain, the V_{L} region of the CD19-binding domain, and the V_{H} region of the BAFF-R binding domain.

Provided herein is a bispecific chimeric antigen receptor (CAR) comprising an extracellular binding domain, a spacer, a transmembrane domain, and an intracellular signaling domain, wherein the extracellular binding domain comprises: a B-cell activating factor receptor (BAFF-R)-binding domain that binds to BAFF-R comprising a heavy chain variable (V_{H}) region and a light chain variable (V_{L}) region; and a CD19-binding domain that binds to CD19 comprising a V_{H} region and a V_{L} region, wherein the extracellular binding domain comprises in order from the amino- to carboxy-terminus: the V_{H} region of the BAFF-R-binding domain, the V_{L} region of the CD19-binding domain, the V_{H} region of the CD19-binding domain, and the V_{L} region of the BAFF-R binding domain.

Provided herein is a bispecific chimeric antigen receptor (CAR) comprising an extracellular binding domain, a spacer, a transmembrane domain, and an intracellular signaling domain, wherein the extracellular binding domain comprises: a B-cell activating factor receptor (BAFF-R)-binding domain that binds to BAFF-R comprising a heavy chain variable (V_{H}) region and a light chain variable (V_{L}) region; and a CD19-binding domain that binds to CD19 comprising a V_{H} region and a V_{L} region, wherein the extracellular binding domain comprises in order from the amino- to carboxy-terminus: the V_{L} region of the BAFF-R-binding domain, the V_{L} region of the CD19-binding domain, the V_{H} region of the CD19-binding domain, and the V_{H} region of the BAFF-R binding domain.

Provided herein is a bispecific chimeric antigen receptor (CAR) comprising an extracellular binding domain, a spacer, a transmembrane domain, and an intracellular signaling domain, wherein the extracellular binding domain comprises: a B-cell activating factor receptor (BAFF-R)-binding domain that binds to BAFF-R comprising a heavy chain variable (V_{H}) region and a light chain variable (V_{L}) region; and a CD19-binding domain that binds to CD19 comprising a V_{H} region and a V_{L} region, wherein the extracellular binding domain comprises in order from the amino- to carboxy-terminus: the V_{H} region of the BAFF-R-binding domain, the V_{H} region of the CD19-binding domain, the V_{L} region of the CD19-binding domain, and the V_{L} region of the BAFF-R binding domain.

Provided herein is a bispecific chimeric antigen receptor (CAR) comprising an extracellular binding domain, a spacer, a transmembrane domain, and an intracellular signaling domain, wherein the extracellular binding domain comprises: a B-cell activating factor receptor (BAFF-R)-binding domain that binds to BAFF-R comprising a heavy chain variable (V_{H}) region and a light chain variable (V_{L}) region; and a CD19-binding domain that binds to CD19 comprising a V_{H} region and a V_{L} region, wherein the extracellular binding domain comprises in order from the amino- to carboxy-terminus: the V_{L} region of the BAFF-R-binding domain, the V_{H} region of the CD19-binding domain, the V_{L} region of the CD19-binding domain, and the V_{H} region of the BAFF-R binding domain.

Provided herein is a bispecific chimeric antigen receptor (CAR) comprising an extracellular binding domain, a spacer, a transmembrane domain, and an intracellular signaling domain, wherein the extracellular binding domain comprises: a B-cell activating factor receptor (BAFF-R)-binding domain that binds to BAFF-R comprising a heavy chain variable (V_{H}) region and a light chain variable (V_{L}) region; and a CD19-binding domain that binds to CD19 comprising a V_{H} region and a V_{L} region, wherein the extracellular binding domain comprises in order from the amino- to carboxy-terminus: (i) the V_{H} region of the BAFF-R-binding domain, the V_{L} region of the CD19-binding domain, the V_{H} region of the CD19-binding domain, and the V_{L} region of the BAFF-R binding domain; or (ii) the V_{L} region of the BAFF-R-binding domain, the V_{L} region of the CD19-binding domain, the V_{H} region of the CD19-binding domain, and the V_{H} region of the BAFF-R binding domain.

In some embodiments, the extracellular binding domain comprises in order from amino- to carboxy-terminus: the V_{L} region of the BAFF-R-binding domain, the V_{L} region of the CD19-binding domain, the V_{H} region of the CD19-binding domain, and the V_{H} region of the BAFF-R-binding domain.

In some embodiments, the extracellular binding domain comprises in order from amino- to carboxy-terminus: the V_{H} region of the BAFF-R-binding domain, the V_{L} region of the CD19-binding domain, the V_{H} region of the CD19-binding domain, and the V_{L} region of the BAFF-R-binding domain.

In some embodiments, the extracellular binding domain comprises in order from amino- to carboxy-terminus: the V_{H} region of the BAFF-R-binding domain, the V_{H} region of the CD19-binding domain, the V_{L} region of the CD19-binding domain, and the V_{L} region of the BAFF-R binding domain.

In some embodiments, the extracellular binding domain comprises in order from amino- to carboxy-terminus: the V_{L} region of the BAFF-R-binding domain, the V_{H} region of the CD19-binding domain, the V_{L} region of the CD19-binding domain, and the V_{H} region of the BAFF-R binding domain.

In some of any embodiments, (i) the V_{H} region of the BAFF-R-binding domain comprises a CDR-H1, a CDR-H2 and a CDR-H3 each comprising a sequence that is contained within SEQ ID NO:1, and the V_{L} region of the BAFF-R-binding domain comprises a CDR-L1, a CDR-L2 and a CDR-L3 each comprising a sequence that is contained within SEQ ID NO:2; (ii) the V_{H} region of the BAFF-R-binding domain comprises a CDR-H1, a CDR-H2 and a CDR-H3 each comprising a sequence that is contained within SEQ ID NO:3, and the V_{L} region of the BAFF-R-binding domain comprises a CDR-L1, a CDR-L2 and a CDR-L3 each having a sequence that is contained within SEQ ID NO:4; (iii) the V_{H} region of the BAFF-R-binding domain comprises a CDR-H1, a CDR-H2 and a CDR-H3 each having a sequence that is contained within SEQ ID NO:5, and the V_{L} region of the BAFF-R-binding domain comprises a CDR-L1, a CDR-L2 and a CDR-L3 each comprising a sequence that is contained within SEQ ID: NO 6; (iv) the V_{H} region of the BAFF-R-binding domain comprises a CDR-H1, a CDR-H2 and a CDR-H3 each comprising a sequence that is contained within SEQ ID NO:7, and the V_{L} region of the BAFF-R-binding domain comprises a CDR-L1, a CDR-L2 and a CDR-L3 each comprising a sequence that is contained within SEQ ID: NO 8; or (v) the V_{H} region of the BAFF-R-binding domain comprises a CDR-H1, a CDR-H2 and a CDR-H3 each comprising a sequence that is contained within SEQ ID NO:9, and the V_{L} region of the BAFF-R-binding domain comprises a CDR-L1, a CDR-L2 and a CDR-L3 each comprising a sequence that is contained within SEQ ID: NO 10.

In some of any embodiments, each CDR is defined in accordance with the Kabat definition, the Chothia definition, the combination of the Kabat and the Chothia definition, the AbM definition, or the contact definition.

Also provided herein is a bispecific CAR comprising an extracellular binding domain, a transmembrane domain, a spacer, and an intracellular signaling domain, wherein the extracellular binding domain comprises: a B-cell activating factor receptor (BAFF-R)-binding domain that binds to BAFF-R comprising a heavy chain variable (V_{H}) region and a light chain variable (V_{L}) region; and a CD19-binding domain that binds to CD19 comprising a V_{H} region and a V_{L} region, wherein: (i) the V_{H} region of the BAFF-R-binding domain comprises a CDR-H1, a CDR-H2 and a CDR-H3 each comprising a sequence that is contained within SEQ ID NO:1, and the V_{L} region of the BAFF-R-binding domain comprises a CDR-L1, a CDR-L2 and a CDR-L3 each comprising a sequence that is contained within SEQ ID NO:2; (ii) the V_{H} region of the BAFF-R-binding domain comprises a CDR-H1, a CDR-H2 and a CDR-H3 each comprising a sequence that is contained within SEQ ID NO:3, and the V_{L} region of the BAFF-R-binding domain comprises a CDR-L1, a CDR-L2 and a CDR-L3 each comprising a sequence that is contained within SEQ ID NO:4; (iii) the V_{H} region of the BAFF-R-binding domain comprises a CDR-H1, a CDR-H2 and a CDR-H3 each comprising a sequence that is contained within SEQ ID NO:5, and the V_{L} region of the BAFF-R-binding domain comprises CDR-L1, CDR-L2 and CDR-L3 each comprising a sequence that is contained within SEQ ID: NO 6; (iv) the V_{H} region of the BAFF-R-binding domain comprises a CDR-H1, a CDR-H2 and a CDR-H3 each comprising a sequence that is contained within SEQ ID NO:7, and the V_{L} region of the BAFF-R-binding domain comprises a CDR-L1, a CDR-L2 and a CDR-L3 each comprising a sequence that is contained within SEQ ID: NO 8; or (v) the V_{H} region of the BAFF-R-binding domain comprises a CDR-H1, a CDR-H2 and a CDR-H3 each comprising a sequence that is contained within SEQ ID NO:9, and the V_{L} region of the BAFF-R-binding domain comprises a CDR-L1, a CDR-L2 and a CDR-L3 each comprising a sequence that is contained within SEQ ID: NO 10.

In some embodiments, each CDR is defined in accordance with the Kabat definition, the Chothia definition, the combination of the Kabat and the Chothia definition, the AbM definition, or the contact definition. In some of any embodiments, (i) the V_{H} region of the BAFF-R-binding domain comprises CDR-H1, CDR-H2 and CDR-H3 sequences set forth in SEQ ID NOS: 16, 17, and 18, respectively; and the V_{L} region of the BAFF-R-binding domain comprises CDR-L1, CDR-L2 and CDR-L3 sequences set forth in SEQ ID NOS: 19, 20, and 21, respectively; (ii) the V_{H} region of the BAFF-R-binding domain comprises CDR-H1, CDR-H2 and CDR-H3 sequences set forth in SEQ ID NOS: 22, 23, and 24, respectively; and the V_{L} region of the BAFF-R-binding domain comprises CDR-L1, CDR-L2 and CDR-L3 sequences set forth in SEQ ID NOS: 25, 26, and 27, respectively; (iii) the V_{H} region of the BAFF-R-binding domain comprises CDR-H1, CDR-H2 and CDR-H3 sequences set forth in SEQ ID NOS: 28, 29, and 30, respectively; and the V_{L} region of the BAFF-R-binding domain comprises CDR-L1, CDR-L2 and CDR-L3 sequences set forth in SEQ ID NOS: 31, 26, and 27, respectively; (iv) the V_{H} region of the BAFF-R-binding domain comprises CDR-H1, CDR-H2 and CDR-H3 sequences set forth in SEQ ID NOS: 22, 32 and 24, respectively; and the V_{L} region of the BAFF-R-binding domain comprises CDR-L1, CDR-L2 and CDR-L3 sequences set forth in SEQ ID NOS: 33, 26, and 34, respectively; or (v) the V_{H} region of the BAFF-R-binding domain comprises CDR-H1, CDR-H2 and CDR-H3 sequences set forth in SEQ ID NOS: 35, 36, and 37, respectively; and the V_{L} region of the BAFF-R-binding domain comprises CDR-L1, CDR-L2 and CDR-L3 sequences set forth in SEQ ID NOS: 38, 39, and 40, respectively.

Also provided herein is a bispecific CAR comprising an extracellular binding domain, a transmembrane domain, a spacer, and an intracellular signaling domain, wherein the extracellular binding domain comprises: a B-cell activating factor receptor (BAFF-R)-binding domain that binds to BAFF-R comprising a heavy chain variable (V_{H}) region and a light chain variable (V_{L}) region; and a CD19-binding domain that binds to CD19 comprising a V_{H} region and a V_{L} region, wherein: (i) the V_{H} region of the BAFF-R-binding domain comprises CDR-H1, CDR-H2 and CDR-H3 sequences set forth in SEQ ID NOS: 16, 17, and 18, respectively; and the V_{L} region of the BAFF-R-binding domain comprises CDR-L1, CDR-L2 and CDR-L3 sequences set forth in SEQ ID NOS: 19, 20, and 21, respectively; (ii) the V_{H} region of the BAFF-R-binding domain comprises CDR-H1, CDR-H2 and CDR-H3 sequences set forth in SEQ ID NOS: 22, 23, and 24, respectively; and the V_{L} region of the BAFF-R-binding domain comprises CDR-L1, CDR-L2 and CDR-L3 sequences set forth in SEQ ID NOS: 25, 26, and 27, respectively; (iii) the V_{H} region of the BAFF-R-binding domain comprises CDR-H1, CDR-H2 and CDR-H3 sequences set forth in SEQ ID NOS: 28, 29, and 30, respectively; and the V_{L} region of the BAFF-R-binding domain comprises CDR-L1, CDR-L2 and CDR-L3 sequences set forth in SEQ ID NOS: 31, 26, and 27, respectively; (iv) the V_{H} region of the BAFF-R-binding domain comprises CDR-H1, CDR-H2 and CDR-H3 sequences set forth in SEQ ID NOS: 22, 32 and 24, respectively; and the V_{L} region of the BAFF-R-binding domain comprises CDR-L1, CDR-L2 and CDR-L3 sequences set forth in SEQ ID NOS: 33, 26, and 34, respectively; or (v) the V_{H} region of the BAFF-R-binding domain comprises CDR-H1, CDR-H2 and CDR-H3 sequences set forth in SEQ ID NOS: 35, 36, and 37, respectively; and the V_{L} region of the BAFF-R-binding domain comprises CDR-L1, CDR-L2 and CDR-L3 sequences set forth in SEQ ID NOS: 38, 39, and 40, respectively.

In some of any embodiments, the extracellular binding domain comprises in order from the amino- to carboxy-terminus: (i) the V_{H} region of the BAFF-R-binding domain, the V_{L} region of the CD19-binding domain, the V_{H} region of the CD19-binding domain, and the V_{L} region of the BAFF-R binding domain; (ii) the V_{L} region of the BAFF-R-binding domain, the V_{L} region of the CD19-binding domain, the V_{H} region of the CD19-binding domain, and the V_{H} region of the BAFF-R binding domain; (iii) the V_{H} region of the BAFF-R-binding domain, the V_{H} region of the CD19-binding domain, the V_{L} region of the CD19-binding domain, and the V_{L} region of the BAFF-R-binding domain; (iv) the V_{L} region of the BAFF-R-binding domain, the V_{H} region of the CD19-binding domain, the V_{L} region of the CD19-binding domain, and the V_{H} region of the BAFF-R binding domain; (v) the V_{L} region of the CD19-binding domain, the V_{H} region of the BAFF-R-binding domain, the V_{L} region of the BAFF-R-binding domain, and the V_{H} region of the CD19-binding domain; (vi) the V_{H} region of the CD19-binding domain, the V_{H} region of the BAFF-R-binding domain, the V_{L} region of the BAFF-R-binding domain, and the V_{L} region of the CD19-binding domain; (vii) the V_{L} region of the CD19-binding domain, the V_{H} region of the CD19-binding domain, the V_{H} region of the BAFF-R-binding domain, and the V_{L} region of the BAFF-R-binding domain; (viii) the V_{L} region of the CD19-binding domain, the V_{H} region of the CD19-binding domain, the V_{L} region of the BAFF-R-binding domain, and the V_{H} region of the BAFF-R-binding domain; (ix) the V_{H} region of the CD19-binding domain, the V_{L} region of the CD19-binding domain, the V_{H} region of the BAFF-R-binding domain, and the V_{L} region of the BAFF-R-binding domain; (x) the V_{H} region of the CD19-binding domain, the V_{L} region of the CD19-binding domain, the V_{L} region of the BAFF-R-binding domain, and the V_{H} region of the BAFF-R-binding domain; (xi) the V_{L} region of the BAFF-R-binding domain, the V_{H} region of the BAFF-R-binding domain, the V_{H} region of the CD19-binding domain, and the V_{L} region of the CD19-binding domain; (xii) the V_{L} region of the BAFF-R-binding domain, the V_{H} region of the BAFF-R-binding domain, the V_{L} region of the CD19-binding domain, and the V_{H} region of the CD19-binding domain; (xiii) the V_{H} region of the BAFF-R-binding domain, the V_{L} region of the BAFF-R-binding domain, the V_{H} region of the CD19-binding domain, and the V_{L} region of the CD19-binding domain; (xiv) the V_{H} region of the BAFF-R-binding domain, the V_{L} region of the BAFF-R-binding domain, the V_{L} region of the CD19-binding domain, and the V_{H} region of the CD19-binding domain; (xv) the V_{L} region of the CD19-binding domain, the V_{L} region of the BAFF-R-binding domain, the V_{H} region of the BAFF-R-binding domain, and the V_{H} region of the CD19-binding domain; or (xvi) the V_{H} region of the CD19-binding domain, the V_{L} region of the BAFF-R-binding domain, the V_{H} region of the BAFF-R-binding domain, and the V_{L} region of the CD19-binding domain.

In some of any embodiments, (i) the V_{H} region of the BAFF-R-binding domain comprises the sequence set forth in, or a sequence that is at least 85%, 86%, 87%, 88%, 89%, 90%, 91%, 92%, 93%, 94%, 95%, 96%, 97%, 98% or 99% identical to, SEQ ID NO:1, and the V_{L} region of the BAFF-R-binding domain comprises the sequence set forth in, or a sequence that is at least 85%, 86%, 87%, 88%, 89%, 90%, 91%, 92%, 93%, 94%, 95%, 96%, 97%, 98% or 99% identical to, SEQ ID NO:2; (ii) the V_{H} region of the BAFF-R-binding domain comprises the sequence set forth in, or a sequence that is at least 85%, 86%, 87%, 88%, 89%, 90%, 91%, 92%, 93%, 94%, 95%, 96%, 97%, 98% or 99% identical to, SEQ ID NO: 3, and the V_{L} region of the BAFF-R-binding domain comprises the sequence set forth in, or a sequence that is at least 85%, 86%, 87%, 88%, 89%, 90%, 91%, 92%, 93%, 94%, 95%, 96%, 97%, 98% or 99% identical to, SEQ ID NO:4; (iii) the V_{H} region of the BAFF-R-binding domain comprises the sequence set forth in, or a sequence that is at least 85%, 86%, 87%, 88%, 89%, 90%, 91%, 92%, 93%, 94%, 95%, 96%, 97%, 98% or 99% identical to, SEQ ID NO:5, and the V_{L} region of the BAFF-R-binding domain comprises the sequence set forth in, or a sequence that is at least 85%, 86%, 87%, 88%, 89%, 90%, 91%, 92%, 93%, 94%, 95%, 96%, 97%, 98% or 99% identical to, SEQ ID NO:6; (iv) the V_{H} region of the BAFF-R-binding domain comprises the sequence set forth in, or a sequence that is at least 85%, 86%, 87%, 88%, 89%, 90%, 91%, 92%, 93%, 94%, 95%, 96%, 97%, 98% or 99% identical to, SEQ ID NO:7, and the V_{L} region of the BAFF-R-binding domain comprises the sequence set forth in, or a sequence that is at least 85%, 86%, 87%, 88%, 89%, 90%, 91%, 92%, 93%, 94%, 95%, 96%, 97%, 98% or 99% identical to, SEQ ID NO:8; or (v) the V_{H} region of the BAFF-R-binding domain comprises the sequence set forth in, or a sequence that is at least 85%, 86%, 87%, 88%, 89%, 90%, 91%, 92%, 93%, 94%, 95%, 96%, 97%, 98% or 99% identical to, SEQ ID NO:9, and the V_{L} region of the BAFF-R-binding domain comprises the sequence set forth in, or a sequence that is at least 85%, 86%, 87%, 88%, 89%, 90%, 91%, 92%, 93%, 94%, 95%, 96%, 97%, 98% or 99% identical to, SEQ ID NO:10.

In some of any embodiments, (i) the V_{H} region of the BAFF-R-binding domain comprises the sequence set forth in SEQ ID NO:1, and the V_{L} region of the BAFF-R-binding domain comprises the sequence set forth in SEQ ID NO:2; (ii) the V_{H} of the BAFF-R-binding domain comprises the sequence set forth in SEQ ID NO:3, and the V_{L} region of the BAFF-R-binding domain comprises the sequence set forth in SEQ ID NO:4; (iii) the V_{H} region of the BAFF-R-binding domain comprises the sequence set forth in SEQ ID NO:5, and the V_{L} region of the BAFF-R-binding domain comprises the sequence set forth in SEQ ID NO:6; (iv) the V_{H} region of the BAFF-R-binding domain comprises the sequence set forth in SEQ ID NO: 7, and the V_{L} region of the BAFF-R-binding domain comprises the sequence set forth in SEQ ID NO:8; or (v) the V_{H} region of the BAFF-R-binding domain comprises the sequence set forth in SEQ ID NO:9, and the V_{L} region of the BAFF-R-binding domain comprises the sequence set forth in SEQ ID NO:10.

In some of any embodiments, the extracellular binding domain comprises, from amino to carboxy terminus: the V_{H} region of the BAFF-R-binding domain, the V_{L} region of the CD19-binding domain, the V_{H} region of the CD19-binding domain, and the V_{L} region of the BAFF-R-binding domain. In some of any embodiments, the extracellular binding domain comprises, from amino to carboxy terminus: the V_{L} region of the BAFF-R-binding domain, the V_{L} region of the CD19-binding domain, the V_{H} region of the CD19-binding domain, and the V_{H} region of the BAFF-R-binding domain.

In some of any embodiments, the extracellular binding domain comprises, from amino to carboxy terminus: the V_{L} region of the CD19-binding domain, the V_{H} region of the BAFF-R-binding domain, the V_{L} region of the BAFF-R-binding domain, and the V_{H} region of the CD19-binding domain. In some of any embodiments, the extracellular binding domain comprises, from amino to carboxy terminus: the V_{H} region of the CD19-binding domain, the V_{H} region of the BAFF-R-binding domain, the V_{L} region of the BAFF-R-binding domain, and the V_{L} region of the CD19-binding domain.

In some of any embodiments, the extracellular binding domain comprises, from amino to carboxy terminus: the V_{L} region of the CD19-binding domain, the V_{H} region of the CD19-binding domain, the V_{H} region of the BAFF-R-binding domain, and the V_{L} region of the BAFF-R-binding domain. In some of any embodiments, the extracellular binding domain comprises, from amino to carboxy terminus: the V_{L} region of the CD19-binding domain, the V_{H} region of the CD19-binding domain, the V_{L} region of the BAFF-R-binding domain, and the V_{H} region of the BAFF-R-binding domain.

In some of any embodiments, the V_{H} region of the BAFF-R-binding domain comprises CDR-H1, CDR-H2 and CDR-H3 sequences set forth in SEQ ID NOS: 22, 23, and 24, respectively; and the V_{L} region of the BAFF-R-binding domain comprises CDR-L1, CDR-L2 and CDR-L3 sequences set forth in SEQ ID NOS: 25, 26, and 27, respectively. In some of any embodiments, the V_{H} region of the BAFF-R-binding domain comprises the sequence set forth in, or a sequence that is at least 85%, 86%, 87%, 88%, 89%, 90%, 91%, 92%, 93%, 94%, 95%, 96%, 97%, 98% or 99% identical to, SEQ ID NO:3, and the V_{L} region of the BAFF-R-binding domain comprises the sequence set forth in, or a sequence that is at least 85%, 86%, 87%, 88%, 89%, 90%, 91%, 92%, 93%, 94%, 95%, 96%, 97%, 98% or 99% identical to, SEQ ID NO:4.

In some of any embodiments, the V_{H} of the BAFF-R-binding domain comprises the sequence set forth in SEQ ID NO:3, and the V_{L} region of the BAFF-R-binding domain comprises the sequence set forth in SEQ ID NO:4. In some of any embodiments, the V_{H} region of the BAFF-R-binding domain comprises the sequence set forth in, or a sequence that is at least 85%, 86%, 87%, 88%, 89%, 90%, 91%, 92%, 93%, 94%, 95%, 96%, 97%, 98% or 99% identical to, SEQ ID NO:5, and the V_{L} region of the BAFF-R-binding domain comprises the sequence set forth in, or a sequence that is at least 85%, 86%, 87%, 88%, 89%, 90%, 91%, 92%, 93%, 94%, 95%, 96%, 97%, 98% or 99% identical to, SEQ ID NO:6

In some of any embodiments, the V_{H} region of the BAFF-R-binding domain comprises CDR-H1, CDR-H2 and CDR-H3 sequences set forth in SEQ ID NOS: 28, 29, and 30, respectively; and the V_{L} region of the BAFF-R-binding domain comprises CDR-L1, CDR-L2 and CDR-L3 sequences set forth in SEQ ID NOS: 31, 26, and 27, respectively. In some of any embodiments, the V_{H} region of the BAFF-R-binding domain comprises the sequence set forth in SEQ ID NO:5, and the V_{L} region of the BAFF-R-binding domain comprises the sequence set forth in SEQ ID NO:6.

In some of any embodiments, the V_{H} region of the BAFF-R-binding domain comprises CDR-H1, CDR-H2 and CDR-H3 sequences set forth in SEQ ID NOS: 16, 17, and 18, respectively; and the V_{L} region of the BAFF-R-binding domain comprises CDR-L1, CDR-L2 and CDR-L3 sequences set forth in SEQ ID NOS: 19, 20, and 21, respectively. In some of any embodiments, the V_{H} region of the BAFF-R-binding domain comprises the sequence set forth in, or a sequence that is at least 85%, 86%, 87%, 88%, 89%, 90%, 91%, 92%, 93%, 94%, 95%, 96%, 97%, 98% or 99% identical to, SEQ ID NO:1, and the V_{L} region of the BAFF-R-binding domain comprises the sequence set forth in, or a sequence that is at least 85%, 86%, 87%, 88%, 89%, 90%, 91%, 92%, 93%, 94%, 95%, 96%, 97%, 98% or 99% identical to, SEQ ID NO:2.

In some of any embodiments, the V_{H} region of the BAFF-R-binding domain comprises the sequence set forth in SEQ ID NO:1, and the V_{L} region of the BAFF-R-binding domain comprises the sequence set forth in SEQ ID NO:2. In some of any embodiments, the V_{H} region of the BAFF-R-binding domain comprises CDR-H1, CDR-H2 and CDR-H3 sequences set forth in SEQ ID NOS: 22, 32 and 24, respectively; and the V_{L} region of the BAFF-R-binding domain comprises CDR-L1, CDR-L2 and CDR-L3 sequences set forth in SEQ ID NOS: 33, 26, and 34, respectively.

In some of any embodiments, the V_{H} region of the BAFF-R-binding domain comprises the sequence set forth in, or a sequence that is at least 85%, 86%, 87%, 88%, 89%, 90%, 91%, 92%, 93%, 94%, 95%, 96%, 97%, 98% or 99% identical to, SEQ ID NO:7, and the V_{L} region of the BAFF-R-binding domain comprises the sequence set forth in, or a sequence that is at least 85%, 86%, 87%, 88%, 89%, 90%, 91%, 92%, 93%, 94%, 95%, 96%, 97%, 98% or 99% identical to, SEQ ID NO:8. In some of any embodiments, the V_{H} region of the BAFF-R-binding domain comprises the sequence set forth in SEQ ID NO:7, and the V_{L} region of the BAFF-R-binding domain comprises the sequence set forth in SEQ ID NO:8.

In some of any embodiments, the V_{H} region of the BAFF-R-binding domain comprises CDR-H1, CDR-H2 and CDR-H3 sequences set forth in SEQ ID NOS: 35, 36, and 37, respectively; and the V_{L} region of the BAFF-R-binding domain comprises CDR-L1, CDR-L2 and CDR-L3 sequences set forth in SEQ ID NOS: 38, 39, and 40, respectively.

In some of any embodiments, the V_{H} region of the BAFF-R-binding domain comprises the sequence set forth in, or a sequence that is at least 85%, 86%, 87%, 88%, 89%, 90%, 91%, 92%, 93%, 94%, 95%, 96%, 97%, 98% or 99% identical to, SEQ ID NO:9, and the V_{L} region of the BAFF-R-binding domain comprises the sequence set forth in, or a sequence that is at least 85%, 86%, 87%, 88%, 89%, 90%, 91%, 92%, 93%, 94%, 95%, 96%, 97%, 98% or 99% identical to, SEQ ID NO:10. In some of any embodiments, the V_{H} region of the BAFF-R-binding domain comprises the sequence set forth in SEQ ID NO:9, and the V_{L} region of the BAFF-R-binding domain comprises the sequence set forth in SEQ ID NO: 10.

In some of any embodiments, the V_{H} region of the BAFF-R binding domain is joined to the V_{L} region of the BAFF-R binding domain via an intradomain linker. In some of any embodiments, the V_{H} region of the CD19-binding domain comprises a CDR-H1, a CDR-H2 and a CDR-H3 each comprising a sequence that is contained within SEQ ID NO:41, and the V_{L} region of the CD19-binding domain comprises a CDR-L1, a CDR-L2 and a CDR-L3 each comprising a sequence that is contained within SEQ ID: NO 42.

In some of any embodiments, each CDR is defined in accordance with the Kabat definition, the Chothia definition, the combination of the Kabat and the Chothia definition, the AbM definition, or the contact definition.

In some of any embodiments, the V_{H} region of the CD19-binding domain comprises CDR-H1, CDR-H2 and CDR-H3 sequences set forth in SEQ ID NOS: 41, 44 and 46, respectively; and the V_{L} region of the CD19-binding domain comprises CDR-L1, CDR-L2 and CDR-L3 sequences set forth in SEQ ID NOS: 47, 49, and 51, respectively. In some of any embodiments, (i) the V_{H} region of the CD19-binding domain comprises the sequences set forth in, or a sequence that is at least 90%, 91%, 92%, 93%, 94%, 95%, 96%, 97%, 98% or 99% identical to, SEQ ID NO: 41; and (ii) the V_{L} region of the CD19-binding domain comprises the sequences set forth in, or a sequence that is at least 90%, 91%, 92%, 93%, 94%, 95%, 96%, 97%, 98% or 99% identical to, SEQ ID NO:42.

In some of any embodiments, the V_{H} region of the CD19-binding domain comprises the sequence set forth in SEQ ID NO: 41; and the V_{L} region of the CD19-binding domain comprises the sequence set forth in SEQ ID NO:42.

Also provided herein is a bispecific CAR comprising an extracellular binding domain, a spacer, a transmembrane domain, and an intracellular signaling domain, wherein the extracellular binding domain comprises: a B-cell activating factor receptor (BAFF-R)-binding domain that binds to BAFF-R comprising a heavy chain variable (V_{H}) region and a light chain variable (V_{L}) region; and a CD19-binding domain that binds to CD19 comprising a V_{H} region and a V_{L} region, wherein the extracellular binding domain comprises in order from amino to carboxy terminus: the V_{H} region of the BAFF-R-binding domain comprising the sequence set forth in SEQ ID NO: 3, the V_{L} region of the CD19-binding domain comprising the sequence set forth in SEQ ID NO: 42, the V_{H} region of the CD19-binding domain comprising the sequence set forth in SEQ ID NO: 41, and the V_{L} region of the BAFF-R-binding domain comprising the sequence set forth in SEQ ID NO: 4.

Also provided herein is a bispecific CAR comprising an extracellular binding domain, a spacer, a transmembrane domain, and an intracellular signaling domain, wherein the extracellular binding domain comprises: a BAFF-R-binding domain comprising V_{H} and V_{L}; and a CD19-binding domain comprising V_{H} and V_{L}, wherein the extracellular binding domain comprises in order from amino to carboxy terminus: the V_{L} region of the BAFF-R-binding domain comprising the sequence set forth in SEQ ID NO: 6, the V_{L} region of the CD19-binding domain comprising the sequence set forth in SEQ ID NO: 42, the V_{H} region of the CD19-binding domain comprising the sequence set forth in SEQ ID NO: 41, and the V_{H} region of the BAFF-R-binding domain comprising the sequence set forth in SEQ ID NO: 5.

In some of any embodiments, the V_{H} region of the CD19-binding domain is joined to the V_{L} region of the CD19-binding domain via an intradomain linker. In some of any embodiments, the intradomain linker is a flexible linker. In some of any embodiments, the intradomain linker is 5 to 25 amino acids in length. In some of any embodiments, the intradomain linker is 12 to 18 amino acids in length. In some of any embodiments, the intradomain linker comprises the sequence set forth in SEQ ID NO:58. In some of any embodiments, the intradomain linker comprises the sequence set forth in SEQ ID NO:59.

In some of any embodiments, the V_{H} region or the V_{L} region of the BAFF-R-binding domain are joined by an interdomain linker to the V_{H} region or the V_{L} region of the CD19-binding domain.

In some of any embodiments, (i) the V_{H} region of the BAFF-R binding domain is joined to the V_{L} region of the CD19-binding domain by an interdomain linker; (i) the V_{H} region of the BAFF-R binding domain is joined to the V_{H} region of the CD19-binding domain by an interdomain linker; (iii) the V_{L} region of the BAFF-R binding domain is joined to the V_{L} region of the CD19-binding domain by an interdomain linker; or (iv) the V_{L} region of the BAFF-R binding domain is joined to the V_{H} region of the CD19-binding domain by an interdomain linker.

In some of any embodiments, the interdomain linker is a flexible peptide linker. In some of any embodiments, the length of the interdomain linker is between 5 and 25 amino acids, inclusive. In some of any embodiments, the length of the interdomain linker is between 5 and 15 amino acids, inclusive. In some of any embodiments, the interdomain linker is a G4S linker (SEQ ID NO:60), a G4S2 linker (SEQ ID NO:61) or a (G4S)4 linker (SEQ ID NO:62). In some of any embodiments, the interdomain linker is set forth in SEQ ID NO: 60. In some of any embodiments, the interdomain linker is set forth in SEQ ID NO: 61.

In some of any embodiments, the spacer is interposed between the extracellular binding domain and the transmembrane domain. In some of any embodiments, the spacer comprises a hinge region sequence. In some of any embodiments, the spacer comprises a hinge region of an immunoglobulin or a variant thereof. In some embodiments, the hinge region of an immunoglobulin is an IgG4 hinge region, optionally a human IgG4 hinge region, or a variant thereof. In some of any embodiments, the spacer comprises a variant IgG4 hinge region comprising substitution of amino acids CPSC to CPPC compared to the wild-type IgG4 hinge region.

In some of any embodiments, the spacer is less than at or about 15 amino acids in length. In some of any embodiments, the spacer is between 12 and 15 amino acids in length. In some of any embodiments, the spacer comprises an amino acid sequence having at least at or about 85%, 86%, 87%, 88%, 89%, 90%, 91%, 92%, 93%, 94%, 95%, 96%, 97%, 98% or 99% identity to SEQ ID NO: 52, optionally wherein the spacer has the sequence set forth in SEQ ID NO:52. In some of any embodiments, the spacer is between 200 and 250 amino acids in length, or between 220 and 240 amino acids in length. In some of any embodiments, the spacer comprises a hinge region of an immunoglobulin, a CH2 region of an immunoglobulin or a chimeric CH2 region of two different immunoglobulins, and a CH3 region of an immunoglobulin. In some embodiments, the spacer comprises an IgG4 hinge region or a variant thereof, a chimeric CH2 region comprising a portion of an IgG4 CH2 and a portion of an IgG2 CH2 (IgG2/4 CH2 region), and an IgG4 CH3 region.

In some of any embodiments, the spacer comprises an amino acid sequence having at least at or about 85%, 86%, 87%, 88%, 89%, 90%, 91%, 92%, 93%, 94%, 95%, 96%, 97%, 98% or 99% identity to SEQ ID NO:53, optionally wherein the spacer has the sequence set forth in SEQ ID NO: 53. In some of any embodiments, the transmembrane domain comprises a transmembrane domain from CD28, optionally a human CD28. In some of any embodiments, the transmembrane domain is or comprises SEQ ID NO: 55 or an amino acid sequence having at least at or about 90%, 91%, 92%, 93%, 94%, 95%, 96%, 97%, 98% or 99% sequence identity to SEQ ID NO: 55. In some of any embodiments, the transmembrane domain is set forth in SEQ ID NO: 55.

In some of any embodiments, the intracellular signaling domain comprises an intracellular signaling domain capable of inducing a primary activation signal in a T cell. In some embodiments, the intracellular signaling domain is a domain from a T cell receptor (TCR) component and/or comprises an immunoreceptor tyrosine-based activation motif (ITAM). In some of any embodiments, the intracellular signaling domain is a cytoplasmic signaling domain of a CD3-zeta (CD3ζ) chain, optionally a human CD3ζ chain.

In some of any embodiments, the intracellular signaling domain comprises the sequence set forth in SEQ ID NO: 57, or an amino acid sequence having at least at or about 90%, 91%, 92%, 93%, 94%, 95%, 96%, 97%, 98% or 99% sequence identity to SEQ ID NO: 57. In some of any embodiments, the intracellular signaling domain is set forth in SEQ ID NO: 57. In some of any embodiments, the intracellular signaling region further comprises a costimulatory signaling region. In some embodiments, the costimulatory signaling region is between the transmembrane region and the intracellular signaling domain.

In some of any embodiments, the costimulatory signaling region comprises an intracellular signaling domain of a T cell costimulatory molecule or a signaling portion thereof. In some of any embodiments, the costimulatory signaling region comprises an intracellular signaling domain of 4-1BB, optionally a human 4-1BB. In some of any embodiments, the costimulatory signaling region comprises the sequence set forth in SEQ ID NO: 56 or an amino acid sequence having at least at or about 90%, 91%, 92%, 93%, 94%, 95%, 96%, 97%, 98% or 99% sequence identity to SEQ ID NO: 56. In some of any embodiments, the costimulatory signaling region is set forth in SEQ ID NO: 56.

Also provided herein is a bispecific CAR comprising the amino acid sequence set forth in SEQ ID NO: 94, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 94.

Also provided herein is a bispecific CAR comprising the amino acid sequence set forth in SEQ ID NO: 95, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 95.

Also provided herein is a bispecific CAR comprising the amino acid sequence set forth in SEQ ID NO: 96, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 96.

Also provided herein is a bispecific CAR comprising the amino acid sequence set forth in SEQ ID NO: 97, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 97.

Also provided herein is a bispecific CAR comprising the amino acid sequence set forth in SEQ ID NO: 98, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 98.

Also provided herein is a bispecific CAR comprising the amino acid sequence set forth in SEQ ID NO: 99, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 99.

Also provided herein is a bispecific CAR comprising the amino acid sequence set forth in SEQ ID NO: 100, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 100.

Also provided herein is a bispecific CAR comprising the amino acid sequence set forth in SEQ ID NO: 101, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 101.

Also provided herein is a bispecific CAR comprising the amino acid sequence set forth in SEQ ID NO: 102, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 102.

Also provided herein is a bispecific CAR comprising the amino acid sequence set forth in SEQ ID NO: 103, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 103.

In some of any embodiments, the binding of the BAFF-R domain to BAFF in the presence of soluble BAFF is reduced no more than 10% relative to binding in the absence of soluble BAFF. In some of any embodiments, the binding of the CD19 domain to CD19 in the presence of FMC63 scFv is reduced no more than 30% relative to binding in the absence of FMC63 scFv.

Also provided is a polynucleotide encoding any of the bispecific CARs provided herein. In some embodiments, the polynucleotide is optimized by splice site elimination. In some of any embodiments, the polynucleotide is codon-optimized for expression in a human cell.

Also provided is a vector comprising any of the polynucleotides provided herein. In some of any embodiments, the vector is a viral vector. In some embodiments, the viral vector is a retroviral vector (e.g., lentiviral vector).

Also provided is a cell comprising any of the bispecific CARs provided herein.

Also provided is a cell comprising any of the polynucleotides provided herein or the vectors provided herein. In some of any embodiments, the cell is an immune cell. In some of any embodiments, the cell is a lymphocyte. In some of any embodiments, the cell is an NK cell or a T cell. In some of any embodiments, the cell is a T cell. In some of any embodiments, the T cell is a CD4+ T cell or a CD8+ T cell. In some of any embodiments, the cell is a primary cell. In some of any embodiments, the cell exhibits cytotoxic activity against CD19+ cells, BAFF-R+ cells, and CD19+/BAFF-R+ cells.

Also provided is a composition comprising a plurality of any of the cells provided herein. In some of any embodiments, the composition further comprises a pharmaceutically acceptable excipient. In some of any embodiments, the composition comprises CD4+ and CD8+ T cells. In some of any embodiments, the ratio of CD4+ to CD8+ T cells is from at or about 1:3 to 3:1, optionally at or about 1:2 to 2:1, optionally at or about 1:1.

In some of any embodiments, the composition may comprise greater than at or about 90%, greater than at or about 95% or greater than at or about 98% of cells in the composition are CD3+ T cells. In some of any embodiments, the composition may comprise at least 40%, at least 50%, at least 60%, at least 70%, at least 80% or at least 90% of cells in the composition express the CAR.

In some of any embodiments, the composition may comprise, among a plurality of the cells in the composition expressing the bispecific CAR, less than at or about 10%, at or about 9%, at or about 8%, at or about 7%, at or about 5%, at or about 4%, at or about 3%, at or about 2% or at or about 1% of the cells in the plurality exhibits tonic signaling and/or antigen independent activity or signaling.

In some of any embodiments, the composition may comprise between about 1.0×10⁷ bispecific CAR-expressing T cells and 1.2×10⁹ bispecific CAR-expressing T cells, between about 1.0×10⁷ bispecific CAR-expressing T cells and 6.5×10⁸ bispecific CAR-expressing T cells, between about 1.5×10⁷ bispecific CAR-expressing T cells and 6.5×10⁸ bispecific CAR-expressing T cells, between about 1.5×10⁷ bispecific CAR-expressing T cells and 6.0×10⁸ bispecific CAR-expressing T cells, between about 2.5×10⁷ bispecific CAR-expressing T cells and 6.0×10⁸ bispecific CAR-expressing T cells, between about 5.0×10⁷ bispecific CAR-expressing T cells and 6.0×10⁸ bispecific CAR-expressing T cells, between about 1.25×10⁷ bispecific CAR-expressing T cells and 1.2×10⁹ bispecific CAR-expressing T cells, between about 1.5×10⁷ bispecific CAR-expressing T cells and 1.2×10⁹ bispecific CAR-expressing T cells, between about 5.0×10⁷ bispecific CAR-expressing T cells and 4.5×10⁸ bispecific CAR-expressing T cells, or between about 1.5×10⁸ bispecific CAR-expressing T cells and 3.0×10⁸ bispecific CAR-expressing T cells, each inclusive.

In some of any embodiments, the composition may comprise at or about 1.5×10⁷, at or about 2.5×10⁷, at or about 5.0×10⁷, at or about 7.5×10⁷, at or about 1.5×10⁸, at or about 2.25×10⁸, at or about 3.0×10⁸, at or about 4.5×10⁸, at or about 6.0×10⁸, at or about 8.0×10⁸, or at or about 1.2×10⁹ bispecific CAR-expressing T cells.

Also provided herein is a method of treating a disease or disorder in a subject, the method comprising administering any of the cells provided herein or any of the compositions provided herein to a subject in need of treatment thereof.

Also provided herein is a method of treatment, comprising administering any of the bispecific CARs provided herein, any of the polynucleotides provided herein, or any of the vectors provided herein to a subject having a disease or disorder. In some of any embodiments, the disease or disorder is a cancer or an autoimmune disease. In some of any embodiments, the disease or disorder is a cancer. In some of any embodiments, the cancer is a BAFF-R-expressing cancer, a CD19-expressing cancer, or a BAFF-R- and CD19-expressing cancer. In some of any embodiments, the cancer is a lymphoma or a leukemia. In some embodiments the lymphoma is a large B cell lymphoma. In some embodiments, the lymphoma is a non-Hodgkin lymphoma.

In some of any embodiments, the disease or disorder is a an autoimmune disease.

Also provided herein is any of the cells provided herein or any of the compositions provided herein for use in treating a disease or disorder. Also provided herein is the use of any of the cells provided herein or any of the compositions provided herein for the manufacture of a medicament for treating a disease or disorder. In some of any embodiments, the disease or disorder is a cancer or autoimmune disease. In some of any embodiments, the disease or disorder is cancer. In some embodiments, the cancer is a BAFF-R-expressing cancer, a CD19-expressing cancer, or a BAFF-R- and CD19-expressing cancer. In some of any embodiments, the cancer is a lymphoma or a leukemia. In some embodiments, the lymphoma is a large B cell lymphoma. In some embodiments, the lymphoma is a non-Hodgkin lymphoma.

In some of any embodiments, the disease or disorder is a an autoimmune disease.

Also provided herein is the use of any of the cells provided herein or any of the compositions provided herein for the treatment of a disease or disorder. Also provided herein is any of the bispecific CARs provided herein, any of the polynucleotides provided herein, or any of the vectors provided herein for use in treating a disease or disorder. Also provided herein is the use of any of the bispecific CARs provided herein, any of the polynucleotides provided herein, or any of the vectors provided herein for the manufacture of a medicament for treating a disease or disorder. Also provided herein is the use of any of the bispecific CARs provided herein, any of the polynucleotides provided herein, or any of the vectors provided herein for the treatment of a disease or disorder.

In some of any embodiments, the disease or disorder is a cancer or autoimmune disease. In some of any embodiments, the disease or disorder is cancer. In some embodiments, the cancer is a BAFF-R-expressing cancer, a CD19-expressing cancer, or a BAFF-R- and CD19-expressing cancer. In some of any embodiments, the cancer is a lymphoma or a leukemia. In some embodiments, the lymphoma is a large B cell lymphoma. In some embodiments, the lymphoma is a non-Hodgkin lymphoma.

In some of any embodiments, the disease or disorder is a an autoimmune disease.

Also provided herein is a kit comprising any of the bispecific CARs provided herein, any of the polynucleotides provided herein, any of the vectors provided herein, any of the cells provided herein, or any of the compositions provided herein, and instructions for use, optionally wherein the instructions are for administering any of the bispecific CARs, any of the cells, or any of the compositions, optionally in accord with the method, any of the cells, any of the compositions, any of the bispecific CARs, any of the polynucleotides, or any of the vectors for any of uses or any of the uses provided herein.

Also provided herein is an article of manufacture comprising any of the bispecific CARs provided herein, any of the polynucleotides provided herein, any of the vectors provided herein, any of the cells provided herein, or any of the compositions provided herein, or any of the kits provided herein.

Also provided herein is an antibody or antigen-binding portion thereof that binds B-cell activating factor receptor (BAFF-R), comprising a heavy chain variable region (V_{H}) and a light chain variable region (V_{L}), wherein (i) V_{H} comprises CDR-H1, CDR-H2, CDR-H3 each having a sequence that is contained within SEQ ID NO:1, and the V_{L} region comprises a light chain complementarity determining region 1 (CDR-L1), a light chain complementarity determining region 2 (CDR-L2) and a light chain complementarity determining region 3 (CDR-L3) contained within SEQ ID NO:2; (ii) the V_{H} region comprises a CDR-H1, a CDR-H2 and a CDR-H3 contained within SEQ ID NO:3, and the V_{L} region comprises a CDR-L1, a CDR-L2 and a CDR-L3 contained within SEQ ID: NO 4; (iii) the V_{H} region comprises a CDR-H1, a CDR-H2 and a CDR-H3 contained within SEQ ID NO:5, and the V_{L} region comprises a CDR-L1, a CDR-L2 and a CDR-L3 contained within SEQ ID: NO 6; (iv) the V_{H} region comprises a CDR-H1, a CDR-H2 and a CDR-H3 contained within SEQ ID NO: 7, and the V_{L} region comprises a CDR-L1, a CDR-L2 and a CDR-L3 contained within SEQ ID: NO 8; or (v) V_{H} comprises a CDR-H1, a CDR-H2 and a CDR-H3 contained within SEQ ID NO:9, and the V_{L} region comprises a CDR-L1, a CDR-L2 and a CDR-L3 contained within SEQ ID: NO 10.

In some embodiments, V_{H} comprises a CDR-H1, a CDR-H2, and a CDR-H3 each having a sequence that is contained within SEQ ID NO: 1, and V_{L} comprises a CDR-L1, a CDR-L2, and a CDR-L3 each having a sequence that is contained within SEQ ID: NO 2. In some embodiments, V_{H} comprises a CDR-H1, a CDR-H2 and a CDR-H3 contained within SEQ ID NO: 3, and V_{L} comprises a CDR-L1, a CDR-L2 and a CDR-L3 contained within SEQ ID: NO 4. In some embodiments, the V_{H} region comprises a CDR-H1, a CDR-H2 and a CDR-H3 contained within SEQ ID NO: 5, and the V_{L} region comprises a CDR-L1, a CDR-L2 and a CDR-L3 contained within SEQ ID: NO 6. In some embodiments, the V_{H} region comprises a CDR-H1, a CDR-H2 and a CDR-H3 contained within SEQ ID NO: 7, and the V_{L} region comprises a CDR-L1, a CDR-L2 and a CDR-L3 contained within SEQ ID: NO 8. In some embodiments, the V_{H} region comprises a CDR-H1, a CDR-H2 and a CDR-H3 contained within SEQ ID NO: 9, and the V_{L} region comprises a CDR-L1, a CDR-L2 and a CDR-L3 contained within SEQ ID: NO 10.

In some of any embodiments, each CDR is defined in accordance with the Kabat definition, the Chothia definition, the combination of the Kabat and the Chothia definition, the AbM definition, or the contact definition. In some of any embodiments, the antibody or antigen-binding portion thereof binds to BAFF-R with a K_{D} of about 10⁻⁷ M to about 10⁻¹¹ M.

Also provided herein is an antibody or antigen-binding portion thereof that specifically binds BAFF-R, comprising V_{H} and V_{L}, wherein: (i) V_{H} comprises a CDR-H1, a CDR-H2, and a CDR-H3 comprising the sequences set forth in SEQ ID NOS: 16, 17, and 18, respectively, and V_{L} comprises a CDR-L1, CDR-L2, and a CDR-L3 comprising the sequences set forth in SEQ ID NOS: 19, 20, and 21, respectively; (ii) V_{H} comprises a CDR-H1, a CDR-H2, and a CDR-H3 comprising the sequences set forth in SEQ ID NOS: 22, 23, and 24, respectively, and V_{L} comprises a CDR-L1, a CDR-L2 and a CDR-L3 comprising the sequences set forth in SEQ ID NOS: 25, 26, and 27, respectively; (iii) V_{H} comprises a CDR-H1, a CDR-H2, and a CDR-H3 comprising the sequences set forth in SEQ ID NOS: 28, 29, and 30, respectively, and V_{L} region comprises a CDR-L1, a CDR-L2 and a CDR-L3 comprising the sequences set forth in SEQ ID NOS: 31, 26, and 27, respectively; (iv) V_{H} comprises a CDR-H1, a CDR-H2, and a CDR-H3 comprising the sequences set forth in SEQ ID NOS: 22, 32 and 24, respectively, and V_{L} comprises a CDR-L1, a CDR-L2 and a CDR-L3 comprising the sequences set forth in SEQ ID NOS: 33, 26, and 34, respectively; or (v) V_{H} comprises a CDR-H1, a CDR-H2, and a CDR-H3 comprising the sequences set forth in SEQ ID NOS: 35, 36, and 37, respectively, and V_{L} comprises a CDR-L1, a CDR-L2, and a CDR-L3 comprising the sequences set forth in SEQ ID NOS: 38, 39, and 40, respectively.

In some of any embodiments, the V_{H} region comprises a CDR-H1, a CDR-H2, and a CDR-H3 comprising the sequence set forth in SEQ ID NOS: 16, 17, and 18, respectively, and the V_{L} region comprises a CDR-L1, a CDR-L2, and a CDR-L3 comprising the sequence set forth in SEQ ID NOS: 19, 20, and 21, respectively. In some embodiments, the V_{H} region comprises a CDR-H1, a CDR-H2, and a CDR-H3 comprising the sequence set forth in SEQ ID NOS: 22, 23, and 24, respectively, and the V_{L} region comprises a CDR-L1, a CDR-L2, and a CDR-L3 comprising the sequence set forth in SEQ ID NOS: 25, 26, and 27, respectively.

In some of any embodiments, the V_{H} region comprises a CDR-H1, a CDR-H2, and a CDR-H3 comprising the sequence set forth in SEQ ID NOS: 28, 29, and 30, respectively, and the V_{L} region comprises a CDR-L1, a CDR-L2, and a CDR-L3 comprising the sequence set forth in SEQ ID NOS: 31, 26, and 27, respectively. In some of any embodiments, the V_{H} region comprises a CDR-H1, a CDR-H2, and a CDR-H3 comprising the sequence set forth in SEQ ID NOS: 22, 32, and 24, respectively, and the V_{L} region comprises a CDR-L1, a CDR-L2, and a CDR-L3 comprising the sequence set forth in SEQ ID NOS: 33, 26, and 34, respectively.

In some of any embodiments, the V_{H} region comprises a CDR-H1, a CDR-H2, and a CDR-H3 comprising the sequence set forth in SEQ ID NOS: 35, 36, and 37, respectively, and the V_{L} region comprises a CDR-L1, a CDR-L2, and a CDR-L3 comprising the sequence set forth in SEQ ID NOS: 38, 39, and 40, respectively.

In some of any embodiments, the (i) V_{H} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 1, and V_{L} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 2; (ii) V_{H} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 3, and V_{L} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 4; (iii) V_{H} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 5, and V_{L} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 6; (iv) V_{H} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 7, and V_{L} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 8; or (v) V_{H} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 9, and V_{L} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 10.

Also provided herein is an antibody or antigen-binding portion thereof that specifically binds BAFF-R, comprising V_{H} and V_{L}, wherein: (i) V_{H} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 1, and V_{L} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 2; (ii) V_{H} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 3, and V_{L} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 4; (iii) V_{H} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 5, and V_{L} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 6; (iv) V_{H} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 7, and V_{L} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 8; or (v) V_{H} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 9, and V_{L} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 10.

In some of any embodiments, the V_{H} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 1, and V_{L} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 2. In some of any embodiments, the V_{H} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 3, and V_{L} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 4.

In some of any embodiments, V_{H} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 5, and V_{L} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 6. In some of any embodiments, V_{H} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 7, and V_{L} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 8.

In some of any embodiments, V_{H} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 9, and V_{L} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 10.

In some of any embodiments, the (i) V_{H} and V_{L} are or comprise the sequence set forth in SEQ ID NOS: 1 and 2, respectively; (ii) V_{H} and V_{L} are or comprise the sequence set forth in SEQ ID NOS: 3 and 4, respectively; (iii) V_{H} and V_{L} are or comprise the sequence set forth in SEQ ID NOS: 5 and 6, respectively; (iv) V_{H} and V_{L} are or comprise the sequence set forth in SEQ ID NOS: 7 and 8, respectively; or (v) V_{H} and V_{L} are or comprise the sequence set forth in SEQ ID NOS: 9 and 10, respectively.

Also provided herein is an antibody or antigen-binding portion thereof that specifically binds BAFF-R, comprising V_{H} and V_{L}, wherein: (i) V_{H} and V_{L} are or comprise the sequence set forth in SEQ ID NOS: 1 and 2, respectively; (ii) V_{H} and V_{L} are or comprise the sequence set forth in SEQ ID NOS: 3 and 4, respectively; (iii) V_{H} and V_{L} are or comprise the sequence set forth in SEQ ID NOS: 5 and 6, respectively; (iv) V_{H} and V_{L} are or comprise the sequence set forth in SEQ ID NOS: 7 and 8, respectively; or (v) V_{H} and V_{L} are or comprise the sequence set forth in SEQ ID NOS: 9 and 10, respectively.

In some of any embodiments, V_{H} and V_{L} are or comprise the sequence set forth in SEQ ID NOS: 1 and 2, respectively. In some of any embodiments, V_{H} and V_{L} are or comprise the sequence set forth in SEQ ID NOS: 3 and 4, respectively. In some of any embodiments, V_{H} and V_{L} are or comprise the sequence set forth in SEQ ID NOS: 5 and 6, respectively. In some of any embodiments, V_{H} and V_{L} are or comprise the sequence set forth in SEQ ID NOS: 7 and 8, respectively. In some of any embodiments, V_{H} and V_{L} are or comprise the sequence set forth in SEQ ID NOS: 9 and 10, respectively.

In some of any embodiments, the antibody is a full-length antibody. In some of any embodiments, the antibody is an antigen-binding fragment. In some of any embodiments, said anti-BAFF-R antibody or antigen-binding portion thereof may be recombinant. In some of any embodiments, V_{H} and V_{L} are human or are derived from a human protein. In some of any embodiments, the antigen-binding portion thereof comprises a single chain variable fragment (scFv).

In some of any embodiments, V_{H} is amino-terminal to V_{L}. In some of any embodiments, V_{H} is carboxy-terminal to V_{L}. In some of any embodiments, V_{H} and V_{L} are joined by a flexible linker. In some of any embodiments, the flexible linker comprises the sequence set forth in SEQ ID NO: 58.

In some of any embodiments, the antigen-binding portion is or comprises the sequence set forth in SEQ ID NO: 11, 12, 13, 14, or 15, or an amino acid sequence having at least at or about 85%, 86%, 87%, 88%, 89%, 90%, 91%, 92%, 93%, 94%, 95%, 96%, 97%, 98% or 99% sequence identity to SEQ ID NO: 11, 12, 13, 14, or 15. In some of any embodiments, the antigen-binding portion is or comprises the sequence set forth in SEQ ID NO: 11. In some of any embodiments, the antigen-binding portion is or comprises the sequence set forth in SEQ ID NO: 12. In some of any embodiments, the antigen-binding portion is or comprises the sequence set forth in SEQ ID NO: 13. In some of any embodiments, the antigen-binding portion is or comprises the sequence set forth in SEQ ID NO: 14. In some of any embodiments, the antigen-binding portion is or comprises the sequence set forth in SEQ ID NO: 15.

In some of any embodiments, said anti-BAFF-R antibody or antigen-binding portion thereof specifically binds to a human B-cell activating factor receptor (BAFF-R) protein. In some of any embodiments, the human BAFF-R protein comprises an amino acid sequence set forth in SEQ ID NO: 120.

Also provided herein is a pharmaceutical composition comprising any of the antibodies or antigen-binding portions provided herein, and a pharmaceutical carrier.

Also provided herein is a chimeric antigen receptor (CAR) comprising any of the extracellular binding domains provided herein comprising any of the antibodies or antigen-binding portions provided herein, a transmembrane domain, and an intracellular signaling domain. In some of any embodiments, the intracellular signaling domain comprises an intracellular signaling domain capable of inducing a primary activation signal in a T cell.

In some of any embodiments, the intracellular signaling domain is a domain from a T cell receptor (TCR) component and/or comprises an immunoreceptor tyrosine-based activation motif (ITAM). In some embodiments, the intracellular signaling domain is a cytoplasmic signaling domain of a CD3-zeta (CD3ζ) chain, optionally a human CD3ζ chain. In some embodiments, the intracellular signaling region further comprises a costimulatory signaling region.

In some of any embodiments, the costimulatory signaling region is between the transmembrane region and the intracellular signaling domain. In some of any embodiments, the costimulatory signaling region comprises an intracellular signaling domain of a T cell costimulatory molecule or a signaling portion thereof. In some of any embodiments, the costimulatory signaling region comprises an intracellular signaling domain of 4-1BB, optionally a human 4-1BB.

Also provided herein is a conjugate, comprising any of the antibodies or antigen-binding portions thereof provided herein and a heterologous molecule or moiety. In some embodiments, the heterologous molecule or moiety is a therapeutic moiety.

Also provided herein is a nucleic acid encoding any of the antibodies or antigen-binding portions provided herein.

Also provided herein is a polynucleotide comprising any of the nucleic acids provided herein.

Also provided herein is a polynucleotide comprising any of the nucleic acids encoding any of the conjugates provided herein. In some of any embodiments, the polynucleotide is optimized by splice site elimination. In some of any embodiments, the polynucleotide is codon-optimized for expression in a human cell.

Also provided herein is an expression vector comprising any of the nucleic acids provided herein.

Also provided herein is a vector, comprising any of the polynucleotides provided herein. In some of any embodiments, the vector is a viral vector. In some of any embodiments, the viral vector is a retroviral vector or a lentiviral vector.

Also provided herein is a cell comprising any of the antibodies or antigen-binding portions thereof provided herein, any of the CARs provided herein, or any of the conjugates provided herein.

Also provided herein is a cell comprising any of the polynucleotides provided herein, or any of the vectors provided herein. In some of any embodiments, the cell is an immune cell. In some of any embodiments, the cell may be a lymphocyte. In some of any embodiments, the cell may be an NK cell or a T cell. In some of any embodiments, the cell is a T cell and the T cell is a CD4+ T cell or a CD8+ T cell. In some of any embodiments, the cell is a primary cell obtained from a subject.

Also provided herein is a composition comprising any of the cells provided herein.

Also provided herein is a composition comprising any of the antibodies or antigen-binding portions thereof provided herein, any of the CARs provided herein, or any of the conjugates provided herein. In some of any embodiments, the composition may further comprise a pharmaceutically acceptable excipient. In some of any embodiments, the composition comprises CD4+ and CD8+ T cells and the ratio of CD4+ to CD8+ T cells is from at or about 1:3 to 3:1, optionally at or about 1:2 to 2:1, optionally at or about 1:1.

Also provided herein is a method of producing an antibody or antigen-binding portion that specifically binds to BAFF-R, comprising culturing any of the host cells provided herein under suitable conditions, and obtaining the product expressed by the host cell.

Also provided herein is a method for preparing a BAFF-R-targeting drug, an anti-BAFF-R antibody-drug conjugate (ADC), a multifunctional anti-BAFF-R antibody, a reagent for diagnosing a tumor expressing BAFF-R, or an anti-BAFF-R chimeric antigen receptor (CAR) modified immune cell, wherein the method comprises providing any of the antibodies or antigen-binding portions provided herein and incorporating said antibody or antigen-binding portion into the BAFF-R-targeting drug, the anti-BAFF-R ADC, the multifunctional anti-BAFF-R antibody, the reagent for diagnosing a tumor expressing BAFF-R, or the anti-BAFF-R chimeric antigen receptor (CAR) modified immune cell.

Also provided herein is a method of treatment, comprising administering any of the cells provided herein or any of the compositions provided herein to a subject having a disease or disorder associated with BAFF-R.

Also provided herein is any of the cells provided herein or any of the compositions provided herein for use in treating a disease or disorder associated with BAFF-R.

Also provided herein is a use of any of the cells provided herein or any of the compositions provided herein for the manufacture of a medicament for treating a disease or disorder associated with BAFF-R.

Also provided herein is a use of any of the cells provided herein or any of the compositions provided herein for the treatment of a disease or disorder associated with BAFF-R.

Also provided herein is a method of treatment, comprising administering any of the antibodies or antigen-binding portions thereof provided herein, any of the pharmaceutical compositions provided herein, any of the CARs provided herein, any of the conjugates provided herein, any of the nucleic acids provided herein, any of the polynucleotides provided herein, or any of the vectors provided herein to a subject having a disease or disorder associated with BAFF-R.

Also provided herein is any of the anti-BAFF-R antibodies or antigen-binding portions thereof provided herein, any of the pharmaceutical compositions provided herein, any of the CARs provided herein, any of the conjugates provided herein, any of the nucleic acids provided herein, any of the polynucleotides provided herein, or any of the vectors provided herein for use in treating a disease or disorder associated with BAFF-R.

Also provided herein is use of any of the anti-BAFF-R antibodies or antigen-binding portions thereof provided herein, any of the pharmaceutical compositions provided herein, any of the CARs provided herein, any of the conjugates provided herein, any of the nucleic acids provided herein, any of the polynucleotides provided herein, or any of the vectors provided herein for the manufacture of a medicament for treating a disease or disorder associated with BAFF-R.

Also provided herein is use of any of the antibodies or antigen-binding portions thereof provided herein, any of the pharmaceutical compositions provided herein, any of the CARs provided herein, any of the conjugates provided herein, any of the nucleic acids provided herein, any of the polynucleotides provided herein, or any of the vectors provided herein for the treatment of a disease or disorder associated with BAFF-R. In some of any embodiments, the disease or disorder associated with BAFF-R is a cancer. In some of any embodiments, the cancer is a BAFF-R-expressing cancer.

Also provided herein is a kit comprising any of the antibodies or antigen-binding portions thereof provided herein, any of the pharmaceutical compositions provided herein, any of the CARs provided herein, any of the conjugates provided herein, any of the nucleic acids provided herein, any of the polynucleotides provided herein, or any of the vectors provided herein, any of the cells provided herein, or any of the compositions provided herein, and instructions for use, optionally wherein the instructions are for administering the antibody or antigen-binding portion thereof, the conjugate, the cell, or the composition, optionally in accord with the method, the cell, composition, antibody or antigen-binding portion thereof, conjugate, polynucleotide, or vector for any use provided herein or for any of the uses provided herein.

Also provided herein is an article of manufacture comprising any of the antibodies or antigen-binding portions thereof provided herein, any of the pharmaceutical compositions provided herein, any of the CARs provided herein, any of the conjugates provided herein, any of the nucleic acids provided herein, any of the polynucleotides provided herein, or any of the vectors provided herein, any of the cells provided herein, any of the compositions provided herein, or any of the kits provided herein.

### BRIEF DESCRIPTION OF THE DRAWINGS

FIG. 1 depicts the activation of Jurkat T cells containing a Nur77 knock-in reporter and constructs encoding various anti-BAFF-R CARs when co-cultured with HEK293 target cells.

FIG. 2 depicts a schematic of the orientation of the BAFF-R and CD19 binders in either a linear bispecific design (left) or a loop bispecific design (right).

FIG. 3 depicts the ability of bispecific CARs to be stimulated by BAFF-R− CD19+ cells, BAFF-R+CD19− cells, neither group of cells, or both groups of cells in Jurkat T cells containing a Nur77 knock-in reporter.

FIGS. 4A-4G depict the cell killing capability (FIGS. 4A, 4C, 4E, and 4F) and the ability to elicit cytokine production (FIGS. 4B, 4D, and 4G) of 30 selected bispecific CARs.

FIGS. 5A-5C depict the cytolytic activity of bispecific CAR-expressing T cells when co-cultured with a B cell cancer cell line (Granta-519). Cytolytic activity of bispecific CAR-expressing T cells co-cultured with Granta cells, which express both BAFF-R and CD19 (FIG. 5A), Granta cells harboring CD19 knock-out (KO) and hence expressing only BAFF-R (FIG. 5B), or Granta cells harboring BAFF-R KO and hence expressing only CD19 (FIG. 5C) at a 1:2 effector: target ratio.

FIGS. 6A-6C depict the levels of cytokine production by T cells expressing various CAR receptors following co-incubation with a B cell cancer cell line (Granta-519). The production of IFNγ (FIG. 6A), IL-2 (FIG. 6B), and TNF-α (FIG. 6C) by the CAR-T cells following co-incubation Granta cells expressing only BAFF-R (CD19 KO), only CD19 (BAFF-R KO), or both BAFF-R and CD19 is depicted.

FIGS. 7A-7B depict expression levels of BAFF-R (FIG. 7A) and CD19 (FIG. 7B) in various cell lines including Granta, Nalm-6, and RL cell lines.

FIG. 8 depicts the effect of soluble BAFF on selected anti-CD19/anti-BAFF-R bispecific CARs.

FIG. 9A-9D depicts the therapeutic effect of selected bispecific CAR T cells in a tumor mouse model. Seven (7) selected bispecific CAR T cells were administered to mice in a Raji (FIG. 9A) or Nalm6 (FIG. 9B) mouse model in two different doses. Four (4) selected bispecific CAR T cells were administered to mice in a Raji (FIG. 9C) or Nalm6 (FIG. 9D) mouse model in two different doses.

### DETAILED DESCRIPTION

Provided herein are bispecific chimeric antigen receptors (CARs) targeting or directed to B-cell activating factor receptor (BAFF-R) and CD19, and BAFF-R- and/or CD19-expressing cells and diseases. Also provided are cells, such as T cells, engineered to express a provided bispecific CAR and compositions containing such cells. BAFF-R is expressed on most mature B cells and in certain diseases or conditions such as B-cell lymphoproliferative disorders. Among the provided embodiments are approaches useful in the treatment of diseases and conditions and/or for targeting such cell types, including nucleic acid molecules that encode BAFF-R- and CD19-binding domains, including chimeric antigen receptors (CARs), and the encoded receptors such as the encoded CARs, and compositions and articles of manufacture comprising the same. The receptors generally can contain antibodies (including antigen-binding antibody fragments, such as heavy chain variable (V_{H}) regions, single domain antibody fragments and single chain fragments, including scFvs, and camelid-derived single domain antibody fragments such as V_{HH} domains) specific for BAFF-R and CD19. Also provided are cells, such as engineered or recombinant cells expressing such BAFF-R- and CD19-binding receptors, e.g., bispecific CARs and/or containing nucleic acids encoding such receptors, and compositions and articles of manufacture and therapeutic doses containing such cells.

The embodiments provided herein relate to CAR T cells targeting both BAFF-R and CD19 for the treatment of cancer, specifically B-cell cancers. While CD19 CAR-T cell therapy has been effective in treating human subjects with B-cell malignancies, a subset of patients will relapse due to CAR-specific antigen loss (“escape”) on the cancerous cells or poor performance of the CAR-T cells. BAFF-R is one of three known receptors for BAFF, a regulator of both B-cell and T-cell function. BAFF-R is a B-cell survival receptor and is highly expressed in B-cell malignancies. The targeting of a second antigen could overcome antigen downregulation or loss thus decreasing the opportunity for immune escape.

Provided are cell therapy approaches utilizing bispecific CARs targeting both BAFF-R and CD19 expressed on autologous primary T cells for use as a therapeutic agent against cancer cells. In some cases, simultaneously targeting both antigens as provided herein may improve the depth and durability of responses across patients, in addition to minimizing relapse due to antigen escape. A mechanism of resistance to CAR T-cell therapies, as evidenced by data from CAR T-cell trials in B-cell malignancies, may be the loss or downregulation (“escape”) of the target antigen. (Robbie G. Majzner and Crystal L. Mackall, Cancer Discov Aug. 22, 2018; DOI 10.1158/2159-8290.CD-18-0442). Such a dual targeting strategy may achieve synergistic or improved tumor responses based on targeting two antigens compared to approaches involving only single antigen targeting. A dual targeting approach may be advantageous to overcome problems due to potential for antigen loss and/or to maximize antigen targeting in cancer.

In some contexts, recombinant receptors can exhibit antigen-independent activity or signaling (also known as “tonic signaling”), which could lead to undesirable effects, such as due to increased differentiation and/or exhaustion of T cells that express the recombinant receptor. In some aspects, such activities may limit the T cell's activity, effect or potency. In some cases, during engineering and ex vivo expansion of the cells for recombinant receptor expression, the cells may exhibit phenotypes indicative of exhaustion, due to tonic signaling through the recombinant receptor. In some cases, alternative or additional cancer-targeted T cell therapy approaches are needed. Among provided chimeric antigen receptors are chimeric receptors that display high expression of both CD19- and BAFF-R-binding domains, as well as low tonic signaling, thereby minimizing possibility of antigen-independent (tonic) signaling. In particular, the bispecific CARs provided herein include CARs with high antigen-dependent activation and minimal tonic signaling.

Among the provided embodiments are approaches useful in the treatment of diseases and conditions and/or for targeting such cell types, including nucleic acid molecules that encode bispecific chimeric antigen receptors (CARs) that bind to both BAFF-R and CD19, and the encoded receptors such as the encoded CARs, and compositions and articles of manufacture comprising the same. The receptors generally can contain antibodies (including antigen-binding antibody fragments, such as heavy chain variable (V_{H}) regions, single domain antibody fragments and single chain fragments, including single chain variable fragments (scFvs)) specific for BAFF-R and CD19. Also provided are cells, such as engineered or recombinant cells expressing such CARs, and/or containing nucleic acids encoding such receptors, and compositions and articles of manufacture and therapeutic doses containing such cells.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. BAFF-R-TARGETING ANTIBODIES

Provided herein are anti-BAFF-R antibodies. Among the antibodies are single-chain antibodies such as those containing a heavy chain variable region (V_{H}) and/or a light chain variable region (V_{L}), or a portion thereof. In some embodiments, the antibodies include a V_{H} and a V_{L}, such as single chain Fv fragments (scFvs). The antibodies include antibodies that specifically bind to BAFF-R, e.g., human BAFF-R. Among the provided anti-BAFF-R antibodies are human antibodies, or antibodies that are modified from or variant of human antibodies. The antibodies include isolated antibodies. Also provided are BAFF-R binding molecules containing such antibodies, such as single-chain proteins, fusion proteins, conjugates and/or recombinant receptors such as chimeric receptors, including antigen receptors. In some aspects, the BAFF-R-binding molecules include isolated molecules.

Also provided are BAFF-R-binding cell surface proteins, such as BAFF-R-binding recombinant receptors. The BAFF-R-binding cell surface proteins can contain the provided antibodies (e.g., antigen-binding antibody fragments) that specifically bind to BAFF-R, such as to BAFF-R proteins, such as human BAFF-R protein. In some examples, the recombinant receptors are chimeric antigen receptors, such as those containing anti-BAFF-R antibodies or antigen-binding fragments thereof.

The term “antibody” herein is used in the broadest sense and includes polyclonal and monoclonal antibodies, including intact antibodies and functional (antigen-binding) antibody fragments, including fragment antigen binding (Fab) fragments, F(ab′)₂ fragments, Fab′ fragments, Fv fragments, recombinant IgG (rlgG) fragments, heavy chain variable (V_{H}) regions capable of specifically binding the antigen, single chain antibody fragments, including single chain variable fragments (scFv), and single domain antibodies (e.g., sdAb, sdFv, nanobody, V_{HH}) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific or trispecific, antibodies, diabodies, triabodies, and tetrabodies, tandem di-scFv, tandem tri-scFv. Unless otherwise stated, the term “antibody” should be understood to encompass functional antibody fragments thereof also referred to herein as “antigen-binding fragments.” The term also encompasses intact or full-length antibodies, including antibodies of any class or sub-class, including IgG and sub-classes thereof, IgM, IgE, IgA, and IgD.

The terms “complementarity determining region,” and “CDR,” synonymous with “hypervariable region” or “HVR,” are known to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4).

The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme); A1-Lazikani et al., J Mol Biol, 1997; 273(4): 927-48 (“Chothia” numbering scheme); MacCallum et al., J. Mol. Biol, 1996; 262:732-745.” (“Contact” numbering scheme); Lefranc M P et al., Dev Comp Immunol, 2003; 27(1): 55-77 (“IMGT” numbering scheme); Honegger A and Plückthun A, J Mol Biol, 2001; 309(3): 657-70, (“Aho” numbering scheme); Martin et al., PNAS, 1989; 86(23): 9268-9272, (“AbM” numbering scheme); and Ye et al., Nucleic Acids Res. 2013; 41 (Web Server issue): W34-40, (“IgBLAST numbering scheme). Details regarding various numbering schemes are also described in, for example, Jarasch et al., Proteins, 2017; 85(1): 65-71; Martin et al., Bioinformatics tools for antibody engineering. In: Dübel, S. (editor) Handbook of Therapeutic Antibodies, Vol. 1. Wiley-VCH, Weinheim, Germany; Martin, A. C. R. (2010). Protein Sequence and Structure Analysis of Antibody Variable Domains. In: kontermann, R., Dübel, S. (eds) Antibody Engineering. Springer Protocols Handbooks. Springer, Berlin, Heidelberg; and Martin, A C R, Antibody Information: How to identify the CDRs by looking at a sequence [online] bioinf.org.uk/abs/info.html, all of which are incorporated by reference in their entireties. Various prediction algorithm tools are available and known for numbering antibody residues and CDRs (e.g., AbYsis, Abnum, AbYmod, AbRSA, IgBLAST, IMGT, or ANARCI).

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, in some cases with insertions. Insertions in the sequence relative to the standard numbering scheme are indicated using insertion letter codes. For example, residues that are inserted between residues L30 and L31 are indicated as L31A, L31B, etc. Deletions in the sequence relative to the standard scheme are accommodated by skipping numbers. The two schemes place certain insertions and deletions (“indels”) at different positions, resulting in differential numbering. For instance, the Chothia numbering scheme is nearly identical to the Kabat numbering scheme, except that insertions are placed at structural positions and topologically equivalents residues do get assigned the same numbers. The Contact scheme is based on analysis of complex crystal structures and is similar in many respects to the Chothia numbering scheme. The AbM scheme is a compromise between Kabat and Chothia definitions based on that used by Oxford Molecular's AbM antibody modeling software. The IgBLAST scheme is based on matching to germline V, D and J genes, and can be determined using National Center for Biotechnology Information (NCBI)'s IgBLAST tool.

In some embodiments, Kabat numbering can be determined by known sequence rules as described in, for example, Kabat et al. (1991), “Sequences of Proteins of Immunological Interest,” 5th Ed. Public Health Service, National Institutes of Health, Bethesda, MD. In some embodiments, the Kabat numbering scheme in some aspects can include any of the following rules to designate CDRs: CDR-L1 starts at approximately residue 24 of the light chain, always has a preceding C residue, and always has a following W residue; the end of CDR-L1 is defined by a stretch of 3 residues, where the W residue can be followed by Y, L, or F, followed by Q or L; CDR-1 has a length of 10 to 17 residues; CDR-L2 always starts 16 residues after the end of CDR-L1; the two residues before CDR-L2 are I and Y but can also be V and Y, I and K, or I and F; CDR-L2 is always 7 residues long; CDR-L3 always starts 33 residues after the end of CDR-L2, always has a preceding C residue, and is strictly followed by a F-G-X-G sequence motif, where X is any amino acid; CDR-L3 has a length of 7 to 11 residues; CDR-H1 starts at approximately position 26 of the heavy chain; the first amino acid in CDR-H1 is always 9 residues after a conserved C residue; CDR-H1 is followed by an invariant W residue followed by typically V, but also can be I or A; CDR-H1 has a length of 5 to 7 residues; CDR-H2 always starts at 15 residues after the end of CDR-H1; the first residue in CDR-H2 is usually preceded by the sequence motif L-E-W-I-G but a number of variations exist; the end of CDR-H2 is defined by a motif of 3 residues—the first residue of the motif of 3 residues can be either K or R, the second residue of the motif of 3 residues can be L, I, V, F, T, or A, the third residue of the motif of 3 residues can be T, S, I, or A; CDR-H2 has a length of 16 to 19 residues; CDR-H3 always starts 33 residues after the end of CDR-H2 and is always 3 residues after a C residue—the first residue of CDR-H3 is preceded by the conserved C residue followed by two residues, which are usually A-R; the residues following CDR-H3 is strictly followed by a W-G-X-G sequence motif, where the X is any amino acid; CDR-H3 typically has a length of 3 to 25 residues; CDR-H3 can be much longer than 25 residues.

In some cases, according to the Chothia numbering scheme, exact boundary positions of certain CDRs can differ based on different definitions for the CDRs (See e.g., Martin, ACR, Antibody Information: How to identify the CDRs by looking at a sequence [online] bioinf.org.uk/abs/info.html). For example, in some instances, the boundary positions for CDR-L1 according to Chothia numbering can be L26-L32 (Chothia et al., Science, 1986; 233(4765): 755-8 and Chothia C. and Lesk A. M. J Mol Biol, 1987; 196(4): 901-17). In some instances, the boundary positions for CDR-L1 can be L25-L32 (Al-Lazikani et al., J Mol Biol, 1997; 273(4): 927-48). In some instances, the boundary positions for CDR-L2 can be L50-L52 and for CDR-L3 can be L91-L96 (Chothia et al., Science, 1986; 233(4765): 755-8; Chothia C. and Lesk A. M. J Mol Biol, 1987; 196(4): 901-17; and Al-Lazikani et al., J Mol Biol, 1997; 273(4): 927-48). In some instances, the boundary positions for CDR-H1 according to Chothia numbering can be H26-H32 (Chothia et al., Science, 1986; 233(4765): 755-8; Chothia C. and Lesk A. M. J Mol Biol, 1987; 196(4): 901-17; and Al-Lazikani et al., J Mol Biol, 1997; 273(4): 927-48). In some instances, the boundary positions for CDR-H2 can be H53-H55 (Chothia et al., Science, 1986; 233(4765): 755-8 and Chothia C. and Lesk A. M. J Mol Biol, 1987, 196(4): 901-17); H52a-H55 (Tramontano et al., J Mol Biol, 1990, 215(1): 175-82), or H52-H56 (Al-Lazikani et al., J Mol Biol., 1997; 273(4): 927-48). In some instances, the boundary positions for CDR-H3 can be H96-H101 (Chothia et al., Science, 1986; 233(4765): 755-8 and Chothia C. and Lesk A. M. J Mol Biol., 1987; 196(4): 901-17). In some instances, the boundary positions for CDR-H3 can be H92-H104 (Morea et al., Biophys Chem, 1997; 68(1-3): 9-16 and Morea et al., J Mol Biol., 1998; 275(2): 269-94).

Table 1, below, exemplifies exemplary numbering and lists exemplary position boundaries of CDR-L1, CDR-L2, CDR-L3 and CDR-H1, CDR-H2, CDR-H3 as identified by Kabat, Chothia, AbM, and Contact schemes, respectively. For CDR-H1, residue numbering is listed using both the Kabat and Chothia numbering schemes. FRs are located between CDRs, for example, with FR-L1 located before CDR-L1, FR-L2 located between CDR-L1 and CDR-L2, FR-L3 located between CDR-L2 and CDR-L3 and so forth. It is noted that because the shown Kabat numbering scheme places insertions at H35A and H35B, the end of the Chothia CDR-H1 loop when numbered using the shown Kabat numbering convention varies between H32 and H34, depending on the length of the loop.

TABLE 1
Boundaries of CDRs according to various numbering schemes.

| CDR | Kabat | Chothia | AbM | Contact |
|---|---|---|---|---|
| CDR-L1 | L24--L34 | L24--L34 | L24--L34 | L30--L36 |
| CDR-L2 | L50--L56 | L50--L56 | L50--L56 | L46--L55 |
| CDR-L3 | L89--L97 | L89--L97 | L89--L97 | L89--L96 |
| CDR-H1 | H31--H35B | H26--H32 . . . 34 | H26--H35B | H30--H35B |
| (Kabat Numbering¹) | | | | |
| CDR-H1 | H31--H35 | H26--H32 | H26--H35 | H30--H35 |
| (Chothia Numbering²) | | | | |
| CDR-H2 | H50--H65 | H52--H56 | H50--H58 | H47--H58 |
| CDR-H3 | H95--H102 | H95--H102 | H95--H102 | H93--H101 |

¹Kabat et al. (1991), “Sequences of Proteins of Immunological Interest,” 5th Ed. Public Health Service, National Institutes of Health, Bethesda, MD
²Al-Lazikani et al., J Mol Biol., 1997; 273(4):927-48).

Thus, unless otherwise specified, a “CDR” or “complementary determining region,” or individual specified CDRs (e.g., CDR-H1, CDR-H2, CDR-H3), of a given antibody or region thereof, such as a variable region thereof, should be understood to encompass a (or the specific) complementary determining region as defined by any of the aforementioned schemes, or other known schemes. For example, where it is stated that a particular CDR (e.g., a CDR-H3) contains the amino acid sequence of a corresponding CDR in a given V_{H} or V_{L} region amino acid sequence, it is understood that such a CDR has a sequence of the corresponding CDR (e.g., CDR-H3) within the variable region, as defined by any of the aforementioned schemes, or other known schemes. In some embodiments, where it is stated that an antibody or antigen-binding fragment thereof comprises a CDR-H1, a CDR-H2, and a CDR-H3 as contained within a given V_{H} region amino acid sequence and a CDR-L1, a CDR-L2, and a CDR-L3 as contained within a given V_{L} region amino acid sequence, the CDRs can be defined by any of the aforementioned schemes, such as Kabat, Chothia, AbM, IgBLAST, IMGT, or Contact method, or other known scheme. In some embodiments, specific CDR sequences are specified. Exemplary CDR sequences of provided antibodies are described using various numbering schemes, although it is understood that a provided antibody can include CDRs as described according to any of the other aforementioned numbering schemes or other known numbering schemes.

Likewise, unless otherwise specified, a FR or individual specified FR(s) (e.g., FR-H1, FR-H2, FR-H3, FR-H4, FR-L1, FR-L2, FR-L3, and/or FR-L4), of a given antibody or region thereof, such as a variable region thereof, should be understood to encompass a (or the specific) framework region as defined by any of the known schemes. In some instances, the scheme for identification of a particular CDR, FR, or FRs or CDRs is specified, such as the CDR as defined by the Kabat, Chothia, AbM, IgBLAST, IMGT, or Contact method, or other known schemes. In other cases, the particular amino acid sequence of a CDR or FR is given. In some embodiments, where it is stated that an antibody or antigen-binding fragment thereof comprises a FR-H1, a FR-H2, a FR-H3, and a FR-H4 as contained within a given V_{H} region amino acid sequence and a FR-L1, a FR-L2, a FR-L3, and a FR-L4 as contained within a given V_{L} region amino acid sequence, the FRs can be defined by any of the aforementioned schemes, such as Kabat, Chothia, AbM, IgBLAST, IMGT, or Contact method, or other known scheme.

The term “variable region” or “variable domain” refers to the domain of an antibody heavy or light chain that is involved in binding the antibody to antigen. The variable regions of the heavy chain and light chain (V_{H} and V_{L}, respectively) of a native antibody generally have similar structures, with each domain comprising four conserved framework regions (FRs) and three CDRs (See, e.g., Kindt et al. Kuby Immunology, 6th ed., W.H. Freeman and Co., page 91 (2007)). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively. See, e.g., Portolano et al., J. Immunol. 150:880-887 (1993); Clarkson et al., Nature 352:624-628 (1991).

Among the provided antibodies are antibody fragments. An “antibody fragment” or “antigen-binding fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; heavy chain variable (V_{H}) regions, single-chain antibody molecules such as scFvs and single-domain antibodies comprising only the V_{H} region; and multispecific antibodies formed from antibody fragments. In some embodiments, the antibody is or comprises an antibody fragment comprising a variable heavy chain (V_{H}) and a variable light chain (V_{L}) region. In particular embodiments, the antibodies are single-chain antibody fragments comprising a heavy chain variable (V_{H}) region and/or a light chain variable (V_{L}) region, such as scFvs.

Single-domain antibodies (sdAbs) are antibody fragments comprising all or a portion of the heavy chain variable region or all or a portion of the light chain variable region of an antibody. In certain embodiments, a single-domain antibody is a human single-domain antibody.

Antibody fragments can be made by various techniques, including but not limited to proteolytic digestion of an intact antibody as well as production by recombinant host cells. In some embodiments, the antibodies are recombinantly-produced fragments, such as fragments comprising arrangements that do not occur naturally, such as those with two or more antibody regions or chains joined by synthetic linkers, e.g., peptide linkers, and/or that are may not be produced by enzyme digestion of a naturally-occurring intact antibody. In some aspects, the antibody fragments are scFvs.

A “humanized” antibody is an antibody in which all or substantially all CDR amino acid residues are derived from non-human CDRs and all or substantially all FR amino acid residues are derived from human FRs. A humanized antibody optionally may include at least a portion of an antibody constant region derived from a human antibody. A “humanized form” of a non-human antibody, refers to a variant of the non-human antibody that has undergone humanization, typically to reduce immunogenicity to humans, while retaining the specificity and affinity of the parental non-human antibody. In some embodiments, some FR residues in a humanized antibody are substituted with corresponding residues from a non-human antibody (e.g., the antibody from which the CDR residues are derived), e.g., to restore or improve antibody specificity or affinity.

Among the provided antibodies are human antibodies. A “human antibody” is an antibody with an amino acid sequence corresponding to that of an antibody produced by a human or a human cell, or non-human source that utilizes human antibody repertoires or other human antibody-encoding sequences, including human antibody libraries. The term excludes humanized forms of non-human antibodies comprising non-human antigen-binding regions, such as those in which all or substantially all CDRs are non-human. The term includes antigen-binding fragments of human antibodies.

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic animals, the endogenous immunoglobulin loci have generally been inactivated. Human antibodies also may be derived from human antibody libraries, including phage display and cell-free libraries, containing antibody-encoding sequences derived from a human repertoire.

Among the provided antibodies are monoclonal antibodies, including monoclonal antibody fragments. The term “monoclonal antibody” as used herein refers to an antibody obtained from or within a population of substantially homogeneous antibodies, i.e., the individual antibodies comprising the population are identical, except for possible variants containing naturally occurring mutations or arising during production of a monoclonal antibody preparation, such variants generally being present in minor amounts. In contrast to polyclonal antibody preparations, which typically include different antibodies directed against different epitopes, each monoclonal antibody of a monoclonal antibody preparation is directed against a single epitope on an antigen. The term is not to be construed as requiring production of the antibody by any particular method. A monoclonal antibody may be made by a variety of techniques, including but not limited to generation from a hybridoma, recombinant DNA methods, phage-display and other antibody display methods.

### A. Exemplary Antibodies

In some embodiments, the antibody, e.g., the anti-BAFF-R antibody, e.g., antigen-binding antibody fragment, contains a heavy and/or light chain variable region (V_{H} or V_{L}) sequence as described, or a sufficient antigen-binding portion thereof. In some embodiments, the antibody, e.g., the anti-BAFF-R antibody, e.g., antigen-binding antibody fragment, is a single chain fragment, such as a single chain Fv (scFv) fragment. In some aspects, the scFv comprises a V_{H} and a V_{L}.

In some embodiments, the anti-BAFF-R antibody, e.g., antigen-binding antibody fragment, contains a V_{H} sequence or sufficient antigen-binding portion thereof that contains a heavy chain complementarity determining region 1 (CDR-H1), a heavy chain complementarity determining region 2 (CDR-H2), and/or a heavy chain complementarity determining region 3 (CDR-H3) as described. In some embodiments, the anti-BAFF-R antibody, e.g., antigen-binding antibody fragment, contains a V_{H} sequence or sufficient antigen-binding portion thereof that contains a CDR-H1, a CDR-H2, and a CDR-H3 as described. In some embodiments, the anti-BAFF-R antibody, e.g., antigen-binding antibody fragment, contains a V_{L} sequence or sufficient antigen-binding portion that contains a light chain complementarity determining region 1 (CDR-L1), a light chain complementarity determining region 2 (CDR-L2), and/or a light chain complementarity determining region 3 (CDR-L3) as described. In some embodiments, the anti-BAFF-R antibody, e.g., antigen-binding antibody fragment, contains a V_{L} sequence or sufficient antigen-binding portion that contains a CDR-L1, a CDR-L2, and a CDR-L3 as described.

In some embodiments, the anti-BAFF-R antibody, e.g., antigen-binding antibody fragment, contains a V_{H} sequence that contains a CDR-H1, a CDR-H2, and/or a CDR-H3 as described and contains a V_{L} sequence that contains a CDR-L1, a CDR-L2, and/or a CDR-L3 as described. In some embodiments, the anti-BAFF-R antibody, e.g., antigen-binding antibody fragment, contains a V_{H} sequence that contains a CDR-H1, a CDR-H2, and a CDR-H3 as described and contains a V_{L} sequence that contains a CDR-L1, a CDR-L2, and a CDR-L3 as described. Also among the antibodies are those having sequences at least at or about 90%, at or about 91%, at or about 92%, at or about 93%, at or about 94%, at or about 95%, at or about 96%, at or about 97%, at or about 98%, or at or about 99% identical to such a sequence.

In some embodiments, the anti-BAFF-R antibody or antigen-binding antibody fragment thereof has a heavy chain variable (V_{H}) region having the amino acid sequence set forth in SEQ ID NO: 1, or an amino acid sequence that has at least at or about 90%, at or about 91%, at or about 92%, at or about 93%, at or about 94%, at or about 95%, at or about 96%, at or about 97%, at or about 98%, or at or about 99% sequence identity to the V_{H} region amino acid set forth in SEQ ID NO: 1, or contains a CDR-H1, CDR-H2, and/or CDR-H3 present in such a V_{H} sequence.

In some embodiments, the anti-BAFF-R antibody or antigen-binding antibody fragment thereof has a heavy chain variable (V_{H}) region having the amino acid sequence set forth in SEQ ID NO: 3, or an amino acid sequence that has at least at or about 90%, at or about 91%, at or about 92%, at or about 93%, at or about 94%, at or about 95%, at or about 96%, at or about 97%, at or about 98%, or at or about 99% sequence identity to the V_{H} region amino acid set forth in SEQ ID NO: 3, or contains a CDR-H1, CDR-H2, and/or CDR-H3 present in such a V_{H} sequence.

In some embodiments, the anti-BAFF-R antibody or antigen-binding antibody fragment thereof has a heavy chain variable (V_{H}) region having the amino acid sequence set forth in SEQ ID NO: 5, or an amino acid sequence that has at least at or about 90%, at or about 91%, at or about 92%, at or about 93%, at or about 94%, at or about 95%, at or about 96%, at or about 97%, at or about 98%, or at or about 99% sequence identity to the V_{H} region amino acid set forth in SEQ ID NO: 5, or contains a CDR-H1, CDR-H2, and/or CDR-H3 present in such a V_{H} sequence.

In some embodiments, the anti-BAFF-R antibody or antigen-binding antibody fragment thereof has a heavy chain variable (V_{H}) region having the amino acid sequence set forth in SEQ ID NO: 7, or an amino acid sequence that has at least at or about 90%, at or about 91%, at or about 92%, at or about 93%, at or about 94%, at or about 95%, at or about 96%, at or about 97%, at or about 98%, or at or about 99% sequence identity to the V_{H} region amino acid set forth in SEQ ID NO: 7, or contains a CDR-H1, CDR-H2, and/or CDR-H3 present in such a V_{H} sequence.

In some embodiments, the anti-BAFF-R antibody or antigen-binding antibody fragment thereof has a heavy chain variable (V_{H}) region having the amino acid sequence set forth in SEQ ID NO: 9, or an amino acid sequence that has at least at or about 90%, at or about 91%, at or about 92%, at or about 93%, at or about 94%, at or about 95%, at or about 96%, at or about 97%, at or about 98%, or at or about 99% sequence identity to the V_{H} region amino acid set forth in SEQ ID NO: 9, or contains a CDR-H1, CDR-H2, and/or CDR-H3 present in such a V_{H} sequence.

In some embodiments, the V_{H} region of an antibody or antigen-binding fragment thereof comprises a CDR-H1, CDR-H2, and/or CDR-H3 according to Kabat numbering. In some embodiments, the V_{H} region of an antibody or antigen-binding fragment thereof comprises a CDR-H1, CDR-H2, and/or CDR-H3 according to Chothia numbering. In some embodiments, the V_{H} region of an antibody or antigen-binding fragment thereof comprises a CDR-H1, CDR-H2, and/or CDR-H3 according to AbM numbering.

In some embodiments, the anti-BAFF-R antibody or antigen-binding antibody fragment thereof comprises a variable heavy chain (V_{H}) region comprising a CDR-H1 comprising the amino acid sequence set forth in SEQ ID NO: 16, a CDR-H2 comprising the amino acid sequence set forth in SEQ ID NO: 17, and a CDR-H3 comprising the amino acid sequence set forth in SEQ ID NO:18. In some embodiments, the antibody or antigen-binding fragment thereof comprises a V_{H} region comprising the amino acid sequence set forth in SEQ ID NO: 16, 17, and 18.

In some embodiments of the antibody or antigen-binding fragment thereof provided herein, the V_{H} region comprises any of the CDR-H1, CDR-H2 and CDR-H3 as described and comprises a framework region 1 (FR1), a FR2, a FR3 and/or a FR4 having at least at or about 90%, at or about 91%, at or about 92%, at or about 93%, at or about 94%, at or about 95%, at or about 96%, at or about 97%, at or about 98%, or at or about 99% sequence identity, respectively, to a FR1, a FR2, a FR3 and/or a FR4 contained within the V_{H} region amino acid sequence set forth in SEQ ID NO: 1. In some embodiments, the antibody or antigen-binding fragment thereof comprises a V_{H} region comprising the amino acid sequence set forth in SEQ ID NO: 1.

In some embodiments, the anti-BAFF-R antibody or antigen-binding antibody fragment thereof comprises a variable heavy chain (V_{H}) region comprising a CDR-H1 comprising the amino acid sequence set forth in SEQ ID NO: 22, a CDR-H2 comprising the amino acid sequence set forth in SEQ ID NO: 23, and a CDR-H3 comprising the amino acid sequence set forth in SEQ ID NO: 24. In some embodiments, the antibody or antigen-binding fragment thereof comprises a V_{H} region comprising the amino acid sequence set forth in SEQ ID NO: 22, 23, and 24.

In some embodiments of the antibody or antigen-binding fragment thereof provided herein, the V_{H} region comprises any of the CDR-H1, CDR-H2 and CDR-H3 as described and comprises a framework region 1 (FR1), a FR2, a FR3 and/or a FR4 having at least at or about 90%, at or about 91%, at or about 92%, at or about 93%, at or about 94%, at or about 95%, at or about 96%, at or about 97%, at or about 98%, or at or about 99% sequence identity, respectively, to a FR1, a FR2, a FR3 and/or a FR4 contained within the V_{H} region amino acid sequence set forth in SEQ ID NO: 3. In some embodiments, the antibody or antigen-binding fragment thereof comprises a V_{H} region comprising the amino acid sequence set forth in SEQ ID NO: 3.

In some embodiments, the anti-BAFF-R antibody or antigen-binding antibody fragment thereof comprises a variable heavy chain (V_{H}) region comprising a CDR-H1 comprising the amino acid sequence set forth in SEQ ID NO: 28, a CDR-H2 comprising the amino acid sequence set forth in SEQ ID NO: 29, and a CDR-H3 comprising the amino acid sequence set forth in SEQ ID NO: 30. In some embodiments, the antibody or antigen-binding fragment thereof comprises a V_{H} region comprising the amino acid sequence set forth in SEQ ID NO: 28, 29, and 30.

In some embodiments of the antibody or antigen-binding fragment thereof provided herein, the V_{H} region comprises any of the CDR-H1, CDR-H2 and CDR-H3 as described and comprises a framework region 1 (FR1), a FR2, a FR3 and/or a FR4 having at least at or about 90%, at or about 91%, at or about 92%, at or about 93%, at or about 94%, at or about 95%, at or about 96%, at or about 97%, at or about 98%, or at or about 99% sequence identity, respectively, to a FR1, a FR2, a FR3 and/or a FR4 contained within the V_{H} region amino acid sequence set forth in SEQ ID NO: 5. In some embodiments, the antibody or antigen-binding fragment thereof comprises a V_{H} region comprising the amino acid sequence set forth in SEQ ID NO: 5.

In some embodiments, the anti-BAFF-R antibody or antigen-binding antibody fragment thereof, has a variable heavy chain (V_{H}) region comprising a CDR-H1 comprising the amino acid sequence set forth in SEQ ID NO: 22, a CDR-H2 comprising the amino acid sequence set forth in SEQ ID NO: 32, and a CDR-H3 comprising the amino acid sequence set forth in SEQ ID NO: 24. In some embodiments, the antibody or antigen-binding fragment thereof comprises a V_{H} region comprising the amino acid sequence set forth in SEQ ID NO: 22, 32, and 24.

In some embodiments of the antibody or antigen-binding fragment thereof provided herein, the V_{H} region comprises any of the CDR-H1, CDR-H2 and CDR-H3 as described and comprises a framework region 1 (FR1), a FR2, a FR3 and/or a FR4 having at least at or about 90%, at or about 91%, at or about 92%, at or about 93%, at or about 94%, at or about 95%, at or about 96%, at or about 97%, at or about 98%, or at or about 99% sequence identity, respectively, to a FR1, a FR2, a FR3 and/or a FR4 contained within the V_{H} region amino acid sequence set forth in SEQ ID NO: 7. In some embodiments, the antibody or antigen-binding fragment thereof comprises a V_{H} region comprising the amino acid sequence set forth in SEQ ID NO: 7.

In some embodiments, the anti-BAFF-R antibody or antigen-binding antibody fragment thereof comprises a variable heavy chain (V_{H}) region comprising a CDR-H1 comprising the amino acid sequence set forth in SEQ ID NO: 35, a CDR-H2 comprising the amino acid sequence set forth in SEQ ID NO: 36, and a CDR-H3 comprising the amino acid sequence set forth in SEQ ID NO: 37. In some embodiments, the antibody or antigen-binding fragment thereof comprises a V_{H} region comprising the amino acid sequence set forth in SEQ ID NO: 35, 36, and 37.

In some embodiments of the antibody or antigen-binding fragment thereof provided herein, the V_{H} region comprises any of the CDR-H1, CDR-H2 and CDR-H3 as described and comprises a framework region 1 (FR1), a FR2, a FR3 and/or a FR4 having at least at or about 90%, at or about 91%, at or about 92%, at or about 93%, at or about 94%, at or about 95%, at or about 96%, at or about 97%, at or about 98%, or at or about 99% sequence identity, respectively, to a FR1, a FR2, a FR3 and/or a FR4 contained within the V_{H} region amino acid sequence set forth in SEQ ID NO: 9. In some embodiments, the antibody or antigen-binding fragment thereof comprises a V_{H} region comprising the amino acid sequence set forth in SEQ ID NO: 9.

In some embodiments, the antibody or antibody fragment comprising a V_{H} region further comprises a light chain or a sufficient antigen binding portion thereof. For example, in some embodiments, the antibody or antigen-binding fragment thereof contains a V_{H} region and a V_{L} region, or a sufficient antigen-binding portion of a V_{H} and V_{L} region. In such embodiments, a V_{H} region sequence can be any of the above described V_{H} sequence. In some such embodiments, the antibody is an antigen-binding fragment, such as a Fab or an scFv. In some such embodiments, the antibody is a full-length antibody that also contains a constant region.

In some embodiments, the anti-BAFF-R antibody, or antigen-binding fragment thereof, contains any of the above V_{H} region and contains a variable light chain region or a sufficient antigen binding portion thereof. For example, in some embodiments, the antibody or antigen-binding fragment thereof contains a V_{H} region and a variable light chain (V_{L}) region, or a sufficient antigen-binding portion of a V_{H} and V_{L} region. In such embodiments, a V_{H} region sequence can be any of the above described V_{H} sequence. In some such embodiments, the antibody is an antigen-binding fragment, such as a Fab or an scFv. In some such embodiments, the antibody is a full-length antibody that also contains a constant region.

In some embodiments, the anti-BAFF-R antibody or antigen-binding fragment thereof has a light chain variable (V_{L}) region having the amino acid sequence set forth in SEQ ID NO: 2, or an amino acid sequence that has at least at or about 90%, at or about 91%, at or about 92%, at or about 93%, at or about 94%, at or about 95%, at or about 96%, at or about 97%, at or about 98%, or at or about 99% sequence identity to the V_{L} region amino acid set forth in SEQ ID NO: 2, or contains a CDR-L1, CDR-L2, and/or CDR-L3 present in such a V_{L} sequence.

In some embodiments, the anti-BAFF-R antibody or antigen-binding antibody fragment thereof has a light chain variable (V_{L}) region having the amino acid sequence set forth in SEQ ID NO: 4, or an amino acid sequence that has at least at or about 90%, at or about 91%, at or about 92%, at or about 93%, at or about 94%, at or about 95%, at or about 96%, at or about 97%, at or about 98%, or at or about 99% sequence identity to the V_{L} region amino acid set forth in SEQ ID NO: 4, or contains a CDR-L1, CDR-L2, and/or CDR-L3 present in such a V_{L} sequence.

In some embodiments, the anti-BAFF-R antibody or antigen-binding antibody fragment thereof has a light chain variable (V_{L}) region having the amino acid sequence set forth in SEQ ID NO: 6, or an amino acid sequence that has at least at or about 90%, at or about 91%, at or about 92%, at or about 93%, at or about 94%, at or about 95%, at or about 96%, at or about 97%, at or about 98%, or at or about 99% sequence identity to the V_{L} region amino acid set forth in SEQ ID NO: 6, or contains a CDR-L1, CDR-L2, and/or CDR-L3 present in such a V_{L} sequence.

In some embodiments, the anti-BAFF-R antibody or antigen-binding antibody fragment thereof has a light chain variable (V_{L}) region having the amino acid sequence set forth in SEQ ID NO: 8, or an amino acid sequence that has at least at or about 90%, at or about 91%, at or about 92%, at or about 93%, at or about 94%, at or about 95%, at or about 96%, at or about 97%, at or about 98%, or at or about 99% sequence identity to the V_{L} region amino acid set forth in SEQ ID NO: 8, or contains a CDR-L1, CDR-L2, and/or CDR-L3 present in such a V_{L} sequence.

In some embodiments, the anti-BAFF-R antibody or antigen-binding antibody fragment thereof has a light chain variable (V_{L}) region having the amino acid sequence set forth in SEQ ID NO: 10, or an amino acid sequence that has at least at or about 90%, at or about 91%, at or about 92%, at or about 93%, at or about 94%, at or about 95%, at or about 96%, at or about 97%, at or about 98%, or at or about 99% sequence identity to the V_{L} region amino acid set forth in SEQ ID NO: 10, or contains a CDR-L1, CDR-L2, and/or CDR-L3 present in such a V_{L} sequence.

In some embodiments, the V_{L} region of an antibody or antigen-binding fragment thereof comprises a CDR-L1, CDR-L2, and/or CDR-L3 according to Kabat numbering. In some embodiments, the V_{L} region of an antibody or antigen-binding fragment thereof comprises a CDR-L1, CDR-L2, and/or CDR-L3 according to Chothia numbering. In some embodiments, the V_{L} region of an antibody or antigen-binding fragment thereof comprises a CDR-L1, CDR-L2, and/or CDR-L3 according to AbM numbering.

In some embodiments, the anti-BAFF-R antibody or antigen-binding antibody fragment thereof comprises a variable light chain (V_{L}) region comprising a CDR-L1 comprising the amino acid sequence set forth in SEQ ID NO: 19, a CDR-L2 comprising the amino acid sequence set forth in SEQ ID NO: 20, and a CDR-L3 comprising the amino acid sequence set forth in SEQ ID NO: 21. In some embodiments, the antibody or antigen-binding fragment thereof comprises a V_{L} region comprising the amino acid sequence set forth in SEQ ID NO: 19, 20, and 21. In some embodiments, the antibody or antigen-binding fragment thereof contains a CDR-L1, CDR-L2, and CDR-L3, respectively, contained within the V_{L} region amino acid sequence set forth in SEQ ID NO: 2.

In some embodiments, the anti-BAFF-R antibody or antigen-binding antibody fragment thereof comprises a variable light chain (V_{L}) region comprising a CDR-L1 comprising the amino acid sequence set forth in SEQ ID NO: 25, a CDR-L2 comprising the amino acid sequence set forth in SEQ ID NO: 26, and a CDR-L3 comprising the amino acid sequence set forth in SEQ ID NO: 27. In some embodiments, the antibody or antigen-binding fragment thereof comprises a V_{L} region comprising the amino acid sequence set forth in SEQ ID NO: 25, 26, and 27. In some embodiments, the antibody or antigen-binding fragment thereof contains a CDR-L1, CDR-L2, and CDR-L3, respectively, contained within the V_{L} region amino acid sequence set forth in SEQ ID NO: 4.

In some embodiments, the anti-BAFF-R antibody or antigen-binding antibody fragment thereof comprises a variable light chain (V_{L}) region comprising a CDR-L1 comprising the amino acid sequence set forth in SEQ ID NO: 31, a CDR-L2 comprising the amino acid sequence set forth in SEQ ID NO: 26, and a CDR-L3 comprising the amino acid sequence set forth in SEQ ID NO: 27. In some embodiments, the antibody or antigen-binding fragment thereof comprises a V_{L} region comprising the amino acid sequence set forth in SEQ ID NO: 31, 26, and 27. In some embodiments, the antibody or antigen-binding fragment thereof contains a CDR-L1, CDR-L2, and CDR-L3, respectively, contained within the V_{L} region amino acid sequence set forth in SEQ ID NO: 6.

In some embodiments, the anti-BAFF-R antibody or antigen-binding antibody fragment thereof comprises a variable light chain (V_{L}) region comprising a CDR-L1 comprising the amino acid sequence set forth in SEQ ID NO: 33, a CDR-L2 comprising the amino acid sequence set forth in SEQ ID NO: 26, and a CDR-L3 comprising the amino acid sequence set forth in SEQ ID NO: 34. In some embodiments, the antibody or antigen-binding fragment thereof comprises a V_{L} region comprising the amino acid sequence set forth in SEQ ID NO: 33, 26, and 34. In some embodiments, the antibody or antigen-binding fragment thereof contains a CDR-L1, CDR-L2, and CDR-L3, respectively, contained within the V_{L} region amino acid sequence set forth in SEQ ID NO: 8.

In some embodiments, the anti-BAFF-R antibody or antigen-binding antibody fragment thereof comprises a variable light chain (V_{L}) region comprising a CDR-L1 comprising the amino acid sequence set forth in SEQ ID NO: 38, a CDR-L2 comprising the amino acid sequence set forth in SEQ ID NO: 39, and a CDR-L3 comprising the amino acid sequence set forth in SEQ ID NO: 40. In some embodiments, the antibody or antigen-binding fragment thereof comprises a V_{L} region comprising the amino acid sequence set forth in SEQ ID NO: 38, 39, and 40. In some embodiments, the antibody or antigen-binding fragment thereof contains a CDR-L1, CDR-L2, and CDR-L3, respectively, contained within the V_{L} region amino acid sequence set forth in SEQ ID NO: 10.

Provided herein is an anti-BAFF-R antibody or antigen-binding fragment thereof comprising a V_{H} region amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 1, and a V_{L} region comprising an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 2.

In some embodiments, the V_{H} region of the antibody or antigen-binding fragment thereof comprises a CDR-H1, a CDR-H2, a CDR-H3, respectively, comprising the amino acid sequences of CDR-H1, CDR-H2, and CDR-H3 contained within the V_{H} region amino acid sequence set forth in SEQ ID NO: 1; and comprises a CDR-L1, a CDR-L2, a CDR-L3, respectively, comprising the amino acid sequences of CDR-L1, CDR-L2, and CDR-L3, respectively contained within the V_{L} region amino acid sequence set forth in SEQ ID NO: 2.

In some embodiments, the V_{H} region of the antibody or antigen-binding fragment thereof comprise the amino acid sequence set forth in SEQ ID NO: 1, and the V_{L} region of the antibody or antigen-binding fragment comprises the amino acid sequence set forth in SEQ ID NO: 2. In some embodiments, the V_{H} and V_{L} regions of the antibody or antigen-binding fragment thereof comprise the amino acid sequences set forth in SEQ ID NO: 1 and 2, respectively, or any antibody or antigen-binding fragment thereof that has at least 90% sequence identity to any of the above V_{H} and V_{L}, such as at least 90%, 91%, 92%, 93%, 94%, 95%, 96%, 97%, 98%, or 99% sequence identity thereto.

For example, the V_{H} and V_{L} regions of the antibody or antigen-binding fragment thereof provided therein comprise the amino acid sequence set forth in SEQ ID NO: 1 and 2, respectively.

Provided herein is an anti-BAFF-R antibody or antigen-binding fragment thereof comprising a V_{H} region amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 3, and a V_{L} region comprising an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 4.

In some embodiments, the V_{H} region of the antibody or antigen-binding fragment thereof comprises a CDR-H1, a CDR-H2, a CDR-H3, respectively, comprising the amino acid sequences of CDR-H1, CDR-H2, and CDR-H3 contained within the V_{H} region amino acid sequence set forth in SEQ ID NO: 3; and comprises a CDR-L1, a CDR-L2, a CDR-L3, respectively, comprising the amino acid sequences of CDR-L1, CDR-L2, and CDR-L3, respectively contained within the V_{L} region amino acid sequence set forth in SEQ ID NO: 4.

In some embodiments, the V_{H} region of the antibody or antigen-binding fragment thereof comprise the amino acid sequence set forth in SEQ ID NO: 3, and the V_{L} region of the antibody or antigen-binding fragment comprises the amino acid sequence set forth in SEQ ID NO: 4. In some embodiments, the V_{H} and V_{L} regions of the antibody or antigen-binding fragment thereof comprise the amino acid sequences set forth in SEQ ID NO: 3 and 4, respectively, or any antibody or antigen-binding fragment thereof that has at least 90% sequence identity to any of the above V_{H} and V_{L}, such as at least 90%, 91%, 92%, 93%, 94%, 95%, 96%, 97%, 98%, or 99% sequence identity thereto.

For example, the V_{H} and V_{L} regions of the antibody or antigen-binding fragment thereof provided therein comprise the amino acid sequence set forth in SEQ ID NO: 3 and 4, respectively.

Provided herein is an anti-BAFF-R antibody or antigen-binding fragment thereof comprising a V_{H} region amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 5, and a V_{L} region comprising an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 6.

In some embodiments, the V_{H} region of the antibody or antigen-binding fragment thereof comprises a CDR-H1, a CDR-H2, a CDR-H3, respectively, comprising the amino acid sequences of CDR-H1, CDR-H2, and CDR-H3 contained within the V_{H} region amino acid sequence set forth in SEQ ID NO: 5; and comprises a CDR-L1, a CDR-L2, a CDR-L3, respectively, comprising the amino acid sequences of CDR-L1, CDR-L2, and CDR-L3, respectively contained within the V_{L} region amino acid sequence set forth in SEQ ID NO: 6.

In some embodiments, the V_{H} region of the antibody or antigen-binding fragment thereof comprise the amino acid sequence set forth in SEQ ID NO: 5, and the V_{L} region of the antibody or antigen-binding fragment comprises the amino acid sequence set forth in SEQ ID NO: 6. In some embodiments, the V_{H} and V_{L} regions of the antibody or antigen-binding fragment thereof comprise the amino acid sequences set forth in SEQ ID NO: 5 and 6, respectively, or any antibody or antigen-binding fragment thereof that has at least 90% sequence identity to any of the above V_{H} and V_{L}, such as at least 90%, 91%, 92%, 93%, 94%, 95%, 96%, 97%, 98%, or 99% sequence identity thereto.

For example, the V_{H} and V_{L} regions of the antibody or antigen-binding fragment thereof provided therein comprise the amino acid sequence set forth in SEQ ID NO: 5 and 6, respectively.

Provided herein is an anti-BAFF-R antibody or antigen-binding fragment thereof comprising a V_{H} region amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 7, and a V_{L} region comprising an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 8.

In some embodiments, the V_{H} region of the antibody or antigen-binding fragment thereof comprises a CDR-H1, a CDR-H2, a CDR-H3, respectively, comprising the amino acid sequences of CDR-H1, CDR-H2, and CDR-H3 contained within the V_{H} region amino acid sequence set forth in SEQ ID NO: 7; and comprises a CDR-L1, a CDR-L2, a CDR-L3, respectively, comprising the amino acid sequences of CDR-L1, CDR-L2, and CDR-L3, respectively contained within the V_{L} region amino acid sequence set forth in SEQ ID NO: 8.

In some embodiments, the V_{H} region of the antibody or antigen-binding fragment thereof comprise the amino acid sequence set forth in SEQ ID NO: 7, and the V_{L} region of the antibody or antigen-binding fragment comprises the amino acid sequence set forth in SEQ ID NO: 8. In some embodiments, the V_{H} and V_{L} regions of the antibody or antigen-binding fragment thereof comprise the amino acid sequences set forth in SEQ ID NO: 7 and 8, respectively, or any antibody or antigen-binding fragment thereof that has at least 90% sequence identity to any of the above V_{H} and V_{L}, such as at least 90%, 91%, 92%, 93%, 94%, 95%, 96%, 97%, 98%, or 99% sequence identity thereto.

For example, the V_{H} and V_{L} regions of the antibody or antigen-binding fragment thereof provided therein comprise the amino acid sequence set forth in SEQ ID NO: 7 and 8, respectively.

Provided herein is an anti-BAFF-R antibody or antigen-binding fragment thereof comprising a V_{H} region amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 9, and a V_{L} region comprising an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 10.

In some embodiments, the V_{H} region of the antibody or antigen-binding fragment thereof comprises a CDR-H1, a CDR-H2, a CDR-H3, respectively, comprising the amino acid sequences of CDR-H1, CDR-H2, and CDR-H3 contained within the V_{H} region amino acid sequence set forth in SEQ ID NO: 9; and comprises a CDR-L1, a CDR-L2, a CDR-L3, respectively, comprising the amino acid sequences of CDR-L1, CDR-L2, and CDR-L3, respectively contained within the V_{L} region amino acid sequence set forth in SEQ ID NO: 10.

In some embodiments, the V_{H} region of the antibody or antigen-binding fragment thereof comprise the amino acid sequence set forth in SEQ ID NO: 9, and the V_{L} region of the antibody or antigen-binding fragment comprises the amino acid sequence set forth in SEQ ID NO: 10. In some embodiments, the V_{H} and V_{L} regions of the antibody or antigen-binding fragment thereof comprise the amino acid sequences set forth in SEQ ID NO: 9 and 10, respectively, or any antibody or antigen-binding fragment thereof that has at least 90% sequence identity to any of the above V_{H} and V_{L}, such as at least 90%, 91%, 92%, 93%, 94%, 95%, 96%, 97%, 98%, or 99% sequence identity thereto.

For example, the V_{H} and V_{L} regions of the antibody or antigen-binding fragment thereof provided therein comprise the amino acid sequence set forth in SEQ ID NO: 9 and 10, respectively.

Provided herein is an anti-BAFF-R antibody or antigen-binding fragment thereof comprising a V_{H} region comprising the sequence set forth in SEQ ID NO: 1 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO: 1; and comprises a V_{L} region comprising the sequence set forth in SEQ ID NO: 2 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO: 2. In some embodiments, the BAFF-R-antibody comprises a V_{H} region that has a CDRH1, a CDRH2 and a CDRH3 comprising the amino acid sequence of SEQ ID NOS: 16, 17, and 18, respectively and a V_{L} region that has a CDRL1, a CDRL2 and a CDRL3 comprising the amino acid sequence of SEQ ID NOS: 19, 20, and 21, respectively. In some embodiments, the V_{H} region comprises the sequence set forth in SEQ ID NO: 1 and the V_{L} region comprises the sequence set forth in SEQ ID NO: 2.

Provided herein is an anti-BAFF-R antibody or antigen-binding fragment thereof comprising a V_{H} region comprising the sequence set forth in SEQ ID NO: 3 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO: 3; and comprises a V_{L} region comprising the sequence set forth in SEQ ID NO: 4 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO: 4. In some embodiments, the BAFF-R-antibody comprises a V_{H} region that has a CDRH1, a CDRH2 and a CDRH3 comprising the amino acid sequence of SEQ ID NOS: 22, 23, and 24, respectively and a V_{L} region that has a CDRL1, a CDRL2 and a CDRL3 comprising the amino acid sequence of SEQ ID NOS: 25, 26, and 27, respectively. In some embodiments, the V_{H} region comprises the sequence set forth in SEQ ID NO: 3 and the V_{L} region comprises the sequence set forth in SEQ ID NO: 4.

Provided herein is an anti-BAFF-R antibody or antigen-binding fragment thereof comprising a V_{H} region comprising the sequence set forth in SEQ ID NO: 5 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO: 5; and comprises a V_{L} region comprising the sequence set forth in SEQ ID NO: 6 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO: 6. In some embodiments, the BAFF-R-antibody comprises a V_{H} region that has a CDRH1, a CDRH2 and a CDRH3 comprising the amino acid sequence of SEQ ID NOS: 28, 29, and 30, respectively and a V_{L} region that has a CDRL1, a CDRL2 and a CDRL3 comprising the amino acid sequence of SEQ ID NOS: 31, 26, and 27, respectively. In some embodiments, the V_{H} region comprises the sequence set forth in SEQ ID NO: 5 and the V_{L} region comprises the sequence set forth in SEQ ID NO: 6.

Provided herein is an anti-BAFF-R antibody or antigen-binding fragment thereof comprising a V_{H} region comprising the sequence set forth in SEQ ID NO: 7 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO: 7; and comprises a V_{L} region comprising the sequence set forth in SEQ ID NO: 8 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO: 8. In some embodiments, the BAFF-R-antibody comprises a V_{H} region that has a CDRH1, a CDRH2 and a CDRH3 comprising the amino acid sequence of SEQ ID NOS: 22, 32, and 24, respectively and a V_{L} region that has a CDRL1, a CDRL2 and a CDRL3 comprising the amino acid sequence of SEQ ID NOS: 33, 26, and 34, respectively. In some embodiments, the V_{H} region comprises the sequence set forth in SEQ ID NO: 7 and the V_{L} region comprises the sequence set forth in SEQ ID NO: 8.

Provided herein is an anti-BAFF-R antibody or antigen-binding fragment thereof comprising a V_{H} region comprising the sequence set forth in SEQ ID NO: 9 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO: 9; and comprises a V_{L} region comprising the sequence set forth in SEQ ID NO: 10 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO: 10. In some embodiments, the BAFF-R-antibody comprises a V_{H} region that has a CDRH1, a CDRH2 and a CDRH3 comprising the amino acid sequence of SEQ ID NOS: 35, 36, and 37, respectively and a V_{L} region that has a CDRL1, a CDRL2 and a CDRL3 comprising the amino acid sequence of SEQ ID NOS: 38, 39, and 40, respectively. In some embodiments, the V_{H} region comprises the sequence set forth in SEQ ID NO: 9 and the V_{L} region comprises the sequence set forth in SEQ ID NO: 10.

In some embodiments, the antibody or antigen-binding fragment thereof is a single-chain antibody fragment, such as a single chain variable fragment (scFv) or a diabody or a single domain antibody (sdAb). In some embodiments, the antibody or antigen-binding fragment is a single domain antibody comprising only the V_{H} region. In some embodiments, the antibody or antigen binding fragment is an scFv comprising a heavy chain variable (V_{H}) region and a light chain variable (V_{L}) region. In some embodiments, the single-chain antibody fragment (e.g., scFv) includes one or more linkers joining two antibody domains or regions, such as a heavy chain variable (V_{H}) region and a light chain variable (V_{L}) region. The linker typically is a peptide linker, e.g., a flexible and/or soluble peptide linker. Among the linkers are those rich in glycine and serine and/or in some cases threonine. In some embodiments, the linkers further include charged residues such as lysine and/or glutamate, which can improve solubility. In some embodiments, the linkers further include one or more proline.

The linker typically is a peptide linker, e.g., a flexible and/or soluble peptide linker, such as one rich in glycine and serine. In some embodiments, the BAFF-R-binding domain comprises a linker between the V_{H} and V_{L} regions. In some embodiments, in order from N- to C-terminus, the BAFF-R-binding domain comprises one of the V_{H} and V_{L} regions, a linker, and the other of the V_{H} and V_{L} regions. In some embodiments, the linker is set forth in SEQ ID NO: 58. Thus, in some embodiments, in order from N- to C-terminus, the BAFF-R-binding domain comprises one of the V_{H} and V_{L} regions, the linker set forth in SEQ ID NO: 58, and the other of the V_{H} and V_{L} regions.

In some aspects, the linkers rich in glycine and serine (and/or threonine) include at least 80%, 85%, 90%, 91%, 92%, 93%, 94%, 95%, 96%, 97%, 98%, or 99% such amino acid(s). In some embodiments, they include at least at or about 50%, 55%, 60%, 70%, or 75%, glycine, serine, and/or threonine. In some embodiments, the linker is comprised substantially entirely of glycine, serine, and/or threonine. The linkers generally are between about 5 and about 50 amino acids in length, typically between at or about 10 and at or about 30, e.g., 10, 11, 12, 13, 14, 15, 16, 17, 18, 19, 20, 21, 22, 23, 24, 25, 26, 27, 28, 29, or 30, and in some examples between 10 and 25 amino acids in length. Exemplary linkers include linkers having various numbers of repeats of the sequence GGGGS (4GS; SEQ ID NO: 60), such as between 2, 3, 4 and 5 repeats of such a sequence. Exemplary linkers include those having or consisting of an sequence set forth in SEQ ID NO: 58 (GGGGSGGGGSGGGGS). Exemplary linkers further include those having or consisting of the sequence set forth in SEQ ID NO: 59 (GSTSGSGKPGSGEGSTKG), SEQ ID NO: 61 (GGGGSGGGGS), and SEQ ID NO: 62 (GGGGGGGGSGGGGSGGGGS).

Accordingly, in some embodiments, the provided embodiments include single-chain antibody fragments, e.g., scFvs, comprising one or more of the aforementioned linkers, such as glycine/serine rich linkers, including linkers having repeats of GGGS (SEQ ID NO: 60), such as the linker set forth in SEQ ID NO: 58, 61, or 62. In some embodiments, the linker comprises the sequence set forth in SEQ ID NO: 58. In some embodiments, the linker comprises the sequence set forth in SEQ ID NO: 60. In some embodiments, the linker comprises the sequence set forth in SEQ ID NO: 61. In some embodiments, the linker comprises the sequence set forth in SEQ ID NO: 62.

In some embodiments, the V_{H} region may be amino terminal to the V_{L} region. In some embodiments, the V_{H} region may be carboxy terminal to the V_{L} region. In particular embodiments, the fragment, e.g., scFv, may include a V_{H} region or portion thereof, followed by the linker, followed by a V_{L} region or portion thereof. In other embodiments, the fragment, e.g., the scFv, may include the V_{L} region or portion thereof, followed by the linker, followed by the V_{H} region or portion thereof.

In some aspects, a BAFF-R scFv provided herein comprises the amino acid sequence set forth in SEQ ID NOs: 11-15, or has an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NOs: 11-15. In some aspects, a BAFF-R scFv provided herein comprises the amino acid sequence set forth in SEQ ID NO: 11, or has an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 11. In some aspects, an scFv provided herein comprises the amino acid sequence set forth in SEQ ID NO: 11. In some aspects, an scFv provided herein comprises an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 11. In some aspects, an scFv provided herein comprises the amino acid sequence set forth in SEQ ID NO: 12, or has an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 12. In some aspects, an scFv provided herein comprises the amino acid sequence set forth in SEQ ID NO: 12. In some aspects, an scFv provided herein comprises an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 12.

In some aspects, a BAFF-R scFv provided herein comprises the amino acid sequence set forth in SEQ ID NO: 13, or has an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 13. In some aspects, an scFv provided herein comprises the amino acid sequence set forth in SEQ ID NO: 13. In some aspects, an scFv provided herein comprises an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 13. In some aspects, an scFv provided herein comprises the amino acid sequence set forth in SEQ ID NO: 14, or has an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 14. In some aspects, an scFv provided herein comprises the amino acid sequence set forth in SEQ ID NO: 14. In some aspects, an scFv provided herein comprises an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 14. In some aspects, a BAFF-R scFv provided herein comprises the amino acid sequence set forth in SEQ ID NO: 15, or has an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 15. In some aspects, an scFv provided herein comprises the amino acid sequence set forth in SEQ ID NO: 15. In some aspects, an scFv provided herein comprises an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 15.

Among the anti-BAFF-R antibodies are human antibodies. In some embodiments of a provided human anti-BAFF-R antibody, e.g., antigen-binding fragments, the human antibody contains a V_{H} region that comprises a portion having at least 95%, 96%, 97%, 98%, 99%, or 100% sequence identity to an amino acid sequence encoded by a germline nucleotide human heavy chain V segment, a portion having at least 95%, 96%, 97%, 98%, 99%, or 100% sequence identity to an amino acid sequence encoded by a germline nucleotide human heavy chain D segment, and/or a portion having at least 95%, 96%, 97%, 98%, 99%, or 100% sequence identity to an amino acid sequence encoded by a germline nucleotide human heavy chain J segment; and/or contains a V_{L} region that comprises a portion having at least 95%, 96%, 97%, 98%, 99%, or 100% sequence identity to an amino acid sequence encoded by a germline nucleotide human kappa or lambda chain V segment, and/or a portion having at least 95%, 96%, 97%, 98%, 99%, or 100% sequence identity to an amino acid sequence encoded by a germline nucleotide human kappa or lambda chain J segment. In some embodiments, the portion of the V_{H} region corresponds to the CDR-H1, CDR-H2 and/or CDR-H3. In some embodiments, the portion of the V_{H} region corresponds to the framework region 1 (FR1), FR2, FR2 and/or FR4. In some embodiments, the portion of the V_{L} region corresponds to the CDR-L1, CDR-L2 and/or CDR-L3. In some embodiments, the portion of the V_{L} region corresponds to the FR1, FR2, FR2 and/or FR4.

In some embodiments, the human antibody, e.g., antigen-binding fragment, contains a CDR-H1 having at least 95%, 96%, 97%, 98%, 99%, or 100% sequence identity to an amino acid sequence of the corresponding CDR-H1 region within a sequence encoded by a germline nucleotide human heavy chain V segment. For example, the human antibody in some embodiments contains a CDR-H1 having a sequence that is 100% identical or with no more than one, two or three amino acid differences as compared to the corresponding CDR-H1 region within a sequence encoded by a germline nucleotide human heavy chain V segment.

In some embodiments, the human antibody, e.g., antigen-binding fragment, contains a CDR-H2 having at least 95%, 96%, 97%, 98%, 99%, or 100% sequence identity to an amino acid sequence of the corresponding CDR-H2 region within a sequence encoded by a germline nucleotide human heavy chain V segment. For example, the human antibody in some embodiments contains a CDR-H2 having a sequence that is 100% identical or with no more than one, two or three amino acid difference as compared to the corresponding CDR-H2 region within a sequence encoded by a germline nucleotide human heavy chain V segment.

In some embodiments, the human antibody, e.g., antigen-binding fragment, contains a CDR-H3 having at least 95%, 96%, 97%, 98%, 99%, or 100% sequence identity to an amino acid sequence of the corresponding CDR-H3 region within a sequence encoded by a germline nucleotide human heavy chain V segment, D segment and J segment. For example, the human antibody in some embodiments contains a CDR-H3 having a sequence that is 100% identical or with no more than one, two or three amino acid differences as compared to the corresponding CDR-H3 region within a sequence encoded by a germline nucleotide human heavy chain V segment, D segment and J segment.

In some embodiments, the human antibody, e.g., antigen-binding fragment, contains a CDR-L1 having at least 95%, 96%, 97%, 98%, 99%, or 100% sequence identity to an amino acid sequence of the corresponding CDR-L1 region within a sequence encoded by a germline nucleotide human light chain V segment. For example, the human antibody in some embodiments contains a CDR-L1 having a sequence that is 100% identical or with no more than one, two or three amino acid differences as compared to the corresponding CDR-L1 region within a sequence encoded by a germline nucleotide human light chain V segment.

In some embodiments, the human antibody, e.g., antigen-binding fragment, contains a CDR-L2 having at least 95%, 96%, 97%, 98%, 99%, or 100% sequence identity to an amino acid sequence of the corresponding CDR-L2 region within a sequence encoded by a germline nucleotide human light chain V segment. For example, the human antibody in some embodiments contains a CDR-L2 having a sequence that is 100% identical or with no more than one, two or three amino acid difference as compared to the corresponding CDR-L2 region within a sequence encoded by a germline nucleotide human light chain V segment.

In some embodiments, the human antibody, e.g., antigen-binding fragment, contains a CDR-L3 having at least 95%, 96%, 97%, 98%, 99%, or 100% sequence identity to an amino acid sequence of the corresponding CDR-L3 region within a sequence encoded by a germline nucleotide human light chain V segment and J segment. For example, the human antibody in some embodiments contains a CDR-L3 having a sequence that is 100% identical or with no more than one, two or three amino acid differences as compared to the corresponding CDR-L3 region within a sequence encoded by a germline nucleotide human light chain V segment and J segment.

In some embodiments, the human antibody, e.g., antigen-binding fragment, contains a framework region that contains human germline gene segment sequences. For example, in some embodiments, the human antibody contains a V_{H} region in which the framework region, e.g. FR1, FR2, FR3 and FR4, has at least 95%, 96%, 97%, 98%, 99%, or 100% sequence identity to a framework region encoded by a human germline antibody segment, such as a V segment and/or J segment. In some embodiments, the human antibody contains a V_{L} region in which the framework region e.g. FR1, FR2, FR3 and FR4, has at least 95%, 96%, 97%, 98%, 99%, or 100% sequence identity to a framework region encoded by a human germline antibody segment, such as a V segment and/or J segment. For example, in some such embodiments, the framework region sequence contained within the V_{H} region and/or V_{L} region differs by no more than 10 amino acids, such as no more than 9, 8, 7, 6, 5, 4, 3, 2 or 1 amino acid, compared to the framework region sequence encoded by a human germline antibody segment.

### B. Immunoconjugates

In some embodiments, the antibody is or is part of an immunoconjugate, in which the antibody is conjugated to one or more heterologous molecule(s), such as, but not limited to, a cytotoxic or an imaging agent. Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins. In some embodiments, the antibody is conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes.

Among the immunoconjugates are antibody-drug conjugates (ADCs), in which an antibody is conjugated to one or more drugs, including but not limited to a maytansinoid (see U.S. Pat. Nos. 5,208,020, 5,416,064 and European Patent EP 0 425 235 B1); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof (see U.S. Pat. Nos. 5,712,374, 5,714,586, 5,739,116, 5,767,285, 5,770,701, 5,770,710, 5,773,001, and 5,877,296; Hinman et al., Cancer Res. 53:3336-3342 (1993); and Lode et al., Cancer Res. 58:2925-2928 (1998)); an anthracycline such as daunomycin or doxorubicin (see Kratz et al., Current Med. Chem. 13:477-523 (2006); Jeffrey et al., Bioorganic & Med. Chem. Letters 16:358-362 (2006); Torgov et al., Bioconj. Chem. 16:717-721 (2005); Nagy et al., Proc. Natl. Acad. Sci. USA 97:829-834 (2000); Dubowchik et al., Bioorg. & Med. Chem. Letters 12:1529-1532 (2002); King et al., J. Med. Chem. 45:4336-4343 (2002); and U.S. Pat. No. 6,630,579); methotrexate; vindesine; a taxane such as docetaxel, paclitaxel, larotaxel, tesetaxel, and ortataxel; a trichothecene; and CC1065.

Also among the immunoconjugates are those in which the antibody is conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

Also among the immunoconjugates are those in which the antibody is conjugated to a radioactive atom to form a radioconjugate. Exemplary radioactive isotopes include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu.

Conjugates of an antibody and cytotoxic agent may be made using any of a number of known protein coupling agents, e.g., linkers, (see Vitetta et al., Science 238:1098 (1987)), WO94/11026. The linker may be a “cleavable linker” facilitating release of a cytotoxic drug in the cell, such as acid-labile linkers, peptidase-sensitive linkers, photolabile linkers, dimethyl linkers, and disulfide-containing linkers (Chari et al., Cancer Res. 52:127-131 (1992); U.S. Pat. No. 5,208,020).

### C. Exemplary Features

In some aspects, the provided antibodies have one or more specified functional features, such as binding properties, including binding to particular epitopes or exhibiting lower or reduced binding to a related but non-specific antigen. In some aspects, the provided antibodies can bind to an epitope that is similar to or overlaps with epitopes of other antibodies, such as reference antibodies, and/or exhibit particular binding affinities. In some aspects, the provided antibodies can bind to an epitope that is different from epitopes of other antibodies, e.g., binding a conformational epitope.

In some embodiments, the provided antibodies or antigen-binding fragment thereof specifically bind to B cell-activating factor receptor (BAFF-R). In some of any of the embodiments provided herein, BAFF-R refers to human BAFF-R. The observation that an antibody or other binding molecule binds to BAFF-R or specifically binds to BAFF-R does not necessarily mean that it binds to BAFF-R from every species. For example, in some embodiments, features of binding to BAFF-R, such as the ability to specifically bind thereto and/or to compete for binding thereto with a reference antibody, and/or to bind with a particular affinity or compete to a particular degree, in some embodiments, refers to the ability with respect to a human BAFF-R and the antibody may not have this feature with respect to a BAFF-R of another species such as mouse. In some embodiments, the antibody binds to human BAFF-R and binds to BAFF-R of another species, such as Rhesus macaque or cynomolgus macaque. In some embodiments, the antibody or an antigen-binding fragment thereof binds to human BAFF-R and does not bind to BAFF-R of another species, such as mouse. In some embodiments, the antibody binds to human BAFF-R and binds to BAFF-R of another species, such as mouse.

In some embodiments, the antibodies, such as the anti-BAFF-R antibodies, e.g., the human antibodies, specifically bind to a particular epitope or region of BAFF-R, such as generally an extracellular epitope or region. In some embodiments, the antibodies or antigen-binding fragment thereof bind, such as specifically bind, to human BAFF-R, such as to one or more epitopes or region of human BAFF-R, or an allelic variant or splice variant thereof. In some embodiments, the antibodies or antigen-binding fragment thereof specifically binds to one or more epitopes within a human BAFF-R.

In some embodiments, the antibodies or antigen-binding fragment thereof bind one or more epitopes of BAFF-R, such as a human BAFF-R. In some embodiments, the antibodies or antigen-binding fragment thereof bind a linear epitope of BAFF-R, such as a human BAFF-R. In some embodiments, the antibodies or antigen-binding fragment thereof bind one or more conformational epitopes of BAFF-R, such as a human BAFF-R.

In some embodiments, the antibody binds to non-human BAFF-R, such as Rhesus macaques (Macaca mulatta) or cynomolgus macaques (Macaca fasicularis). In some aspects, the extracellular binding domain of the non-human BAFF-R is at least 99% identical to the human BAFF-R sequence. In some embodiments, the antibody binds to non-human BAFF-R, such as monkey, rabbit, rat, mouse, or other species of BAFF-R. In some embodiments, the antibody binds to mouse (Mus musculus) BAFF-R, such as to an epitope or region of mouse BAFF-R. In some embodiments, the antibody binds to human BAFF-R and binds to mouse BAFF-R. In some of any of the provided embodiments, the antibody or antigen-binding fragment thereof does not bind to, is not cross-reactive to, or binds at a lower extent, level or degree or affinity to a non-human BAFF-R.

In some embodiments, the provided antibodies are capable of binding BAFF-R, such as human BAFF-R, with at least a certain affinity, as measured by any of a number of known methods. In some embodiments, the affinity is represented by an equilibrium dissociation constant (K_{D}). In some embodiments, the affinity is represented by EC₅₀.

A variety of assays are known for assessing binding affinity, equilibrium dissociation constant (K_{D}), equilibrium association constant (K_{A}), EC₅₀, on-rate (association rate constant; kₒₙ or kₐ; units of 1/Ms or M⁻¹ s⁻¹) and the off-rate (dissociation rate constant; k_{off} or k_{d}; units of l/s or s⁻¹) and/or determining whether a binding molecule (e.g., an antibody or fragment thereof) specifically binds to a particular ligand (e.g., an antigen, such as BAFF-R). One can determine the binding affinity of a binding molecule, e.g., an antibody or an antigen-binding fragment thereof, for an antigen, e.g., BAFF-R, such as human BAFF-R or cynomolgus BAFF-R or mouse BAFF-R, such as by using any of a number of binding assays that are well known. For example, in some embodiments, a BIAcore® instrument can be used to determine the binding kinetics and constants of a complex between two proteins (e.g., an antibody or fragment thereof, and an antigen, such as BAFF-R), using surface plasmon resonance (SPR) analysis (see, e.g., Scatchard et al., Ann. N.Y. Acad. Sci. 51:660, 1949; Wilson, Science 295:2103, 2002; Wolff et al., Cancer Res. 53:2560, 1993; and U.S. Pat. Nos. 5,283,173, 5,468,614, or the equivalent).

SPR measures changes in the concentration of molecules at a sensor surface as molecules bind to or dissociate from the surface. The change in the SPR signal is directly proportional to the change in mass concentration close to the surface, thereby allowing measurement of binding kinetics between two molecules. The dissociation rate constant (k_{off} or k_{d}), the association rate constant (kₒₙ or kₐ) and/or equilibrium dissociation constant (K_{D}) and/or equilibrium association constant (K_{A}) for the complex can be determined by monitoring changes in the refractive index with respect to time as buffer is passed over the chip. Other suitable assays for measuring the binding of one protein to another include, for example, immunoassays such as enzyme linked immunosorbent assays (ELISA) and radioimmunoassays (RIA), or determination of binding by monitoring the change in the spectroscopic or optical properties of the proteins through fluorescence, UV absorption, circular dichroism, or nuclear magnetic resonance (NMR). Other exemplary assays include, but are not limited to, Western blot, ELISA, analytical ultracentrifugation, spectroscopy, flow cytometry, sequencing, genetic reporter assays, flow cytometry, and other methods for detection of expressed nucleic acids or binding of proteins.

In some embodiments, the binding molecule, e.g., antibody or fragment thereof, binds, such as specifically binds, to an antigen, e.g., BAFF-R or an epitope therein, with an affinity or K_{A} (i.e., an equilibrium association constant of a particular binding interaction with units of 1/M or M⁻¹; equal to the ratio of the on-rate [kₒₙ or kₐ] to the off-rate [k_{off} or k_{d}] for this association reaction, assuming bimolecular interaction) equal to or greater than 10⁵ M⁻¹. In some embodiments, the peptide binding molecule binds, such as specifically binds, to an epitope of an antigen, e.g., human BAFF-R, with an affinity or K_{A} (i.e., an equilibrium association constant of a particular binding interaction with units of 1/M or M⁻¹) equal to or greater than 10⁵ M⁻¹ (which equals the ratio of the on-rate [kₒₙ] to the off-rate [k_{off}] for this association reaction). In some embodiments, the binding molecule, e.g., antibody or antigen-binding fragment thereof, exhibits a binding affinity for a T cell epitope of the target polypeptide with an affinity or K_{A} ranging from at or about 10⁶ M⁻¹ to at or about 10¹⁰ M⁻¹, such as from at or about 10⁶ M⁻¹ to at or about 10⁹ M⁻¹, or from at or about 10⁶ M⁻¹ to at or about 10⁸ M⁻¹. In some embodiments, binding affinity may be classified as high affinity or as low affinity. For example, in some cases, a binding molecule, e.g., antibody or antigen-binding fragment thereof, that exhibits high affinity binding to a particular epitope interacts with such epitope with a K_{A} of at least at or about 10⁷ M⁻¹, at least at or about 10⁸ M⁻¹, at least at or about 10⁹ M¹, at least at or about 10¹⁰ M⁻¹, at least at or about 10¹¹ M⁻¹, at least at or about 10¹² M⁻¹, or at least at or about 10¹³ M⁻¹. In some cases, a binding molecule, e.g., antibody or antigen-binding fragment thereof, that exhibits low affinity binding exhibits a K_{A} of up to 10⁷ M¹, up to 10⁶ M⁻¹, up to 10⁵ M⁻¹.

Alternatively, affinity can be defined as an equilibrium dissociation constant (K_{D}) of a particular binding interaction with units of M (e.g., 10⁻⁵ M to 10⁻¹³ M). In some embodiments, the antibody or fragment thereof exhibits a binding affinity for the epitope with a K_{D} (i.e., an equilibrium dissociation constant of a particular binding interaction with units of M; equal to the ratio of the off-rate [k_{off} or k_{d}] to the on-rate [kₒₙ or kₐ] for this association reaction, assuming bimolecular interaction) of equal to or less than 10⁻⁵ M. For example, the equilibrium dissociation constant K_{D} can range from 10⁻⁵ M to 10⁻¹³ M, such as 10⁻⁷ M to 10⁻¹¹ M, 10⁻⁷ M to 10⁻¹⁰ M, 10⁻⁷ M to 10⁻⁹ M, 10⁻⁸ M to 10⁻¹⁰ M, or 10⁻⁹ M to 10⁻¹⁰ M.

The on-rate (association rate constant; kₒₙ or kₐ; units of 1/Ms or M⁻¹ s⁻¹) and the off-rate (dissociation rate constant; k_{off} or k_{d}; units of l/s or s⁻¹) can be determined using any of the known assay methods, for example, surface plasmon resonance (SPR), or other methods described herein for measuring the binding of one protein to another.

In some embodiments, the binding affinity (EC₅₀) and/or the equilibrium dissociation constant (K_{D}) of the antibody to BAFF-R, such as human BAFF-R, is from at or about 0.1 nM to at or about 500 nM, from at or about 0.1 nM to at or about 100 nM, from at or about 0.1 nM to at or about 50 nM, from at or about 0.1 nM to at or about 10 nM, from at or about 0.1 nM to at or about 1 nM, from at or about 1 nM to at or about 500 nM, from at or about 1 nM to at or about 100 nM, from at or about 1 nM to at or about 50 nM, from at or about 1 nM to at or about 10 nM, from at or about 10 nM to at or about 500 nM, from at or about 10 nM to at or about 100 nM, from at or about 10 nM to at or about 50 nM, from at or about 50 nM to at or about 500 nM, from at or about 50 nM to at or about 100 nM or from at or about 100 nM to at or about 500 nM. In certain embodiments, the binding affinity (EC₅₀) and/or the equilibrium dissociation constant (K_{D}) of the antibody to BAFF-R, such as human BAFF-R, is at or about or less than at or about 100 nM, 50 nM, 40 nM, 30 nM, 25 nM, 20 nM, 19 nM, 18 nM, 17 nM, 16 nM, 15 nM, 14 nM, 13 nM, 12 nM, 11 nM, 10 nM, 9 nM, 8 nM, 7 nM, 6 nM, 5 nM, 4 nM, 3 nM, 2 nM, or 1 nM, or a range defined by any of the foregoing. In some embodiments, the antibodies bind to BAFF-R, such as human BAFF-R, with a sub-nanomolar binding affinity, for example, with a binding affinity less than at or about 1 nM, such as less than at or about 0.9 nM, 0.8 nM, 0.7 nM, 0.6 nM, 0.5 nM, 0.4 nM, 0.3 nM, 0.2 nM or 0.1 nM. In some embodiments, the binding affinity (EC₅₀) and/or the equilibrium dissociation constant, K_{D}, of the binding molecule, e.g., anti-BAFF-R antibody or fragment thereof, to BAFF-R, such as human BAFF-R, is from at or about 0.01 nM to about 1 μM, 0.1 nM to 1 μM, 1 nM to 1 μM, 1 nM to 500 nM, 1 nM to 100 nM, 1 nM to 50 nM, 1 nM to 10 nM, 10 nM to 500 nM, 10 nM to 100 nM, 10 nM to 50 nM, 50 nM to 500 nM, 50 nM to 100 nM or 100 nM to 500 nM. In certain embodiments, the binding affinity (EC₅₀) and/or the equilibrium dissociation constant, K_{D}, of the binding molecule, e.g., anti-BAFF-R antibody or fragment thereof, to BAFF-R, such as a human BAFF-R, is at or about or less than at or about 1 μM, 500 nM, 100 nM, 50 nM, 40 nM, 30 nM, 25 nM, 20 nM, 19 nM, 18 nM, 17 nM, 16 nM, 15 nM, 14 nM, 13 nM, 12 nM, 11 nM, 10 nM, 9 nM, 8 nM, 7 nM, 6 nM, 5 nM, 4 nM, 3 nM, 2 nM, or 1 nM or less, or a range defined by any of the foregoing.

In some embodiments, the EC₅₀ and/or the K_{D} of the binding molecule, e.g., anti-BAFF-R antibody or fragment thereof, to BAFF-R, is between at or about 10 nM and at or about 90 nM, between at or about 20 nM and at or about 80 nM, between at or about 30 nM and at or about 70 nM, between at or about 40 nM and at or about 60 nM, or between at or about 40 nM and at or about 50 nM. In certain embodiments, the EC₅₀ and/or the K_{D} of the binding molecule, e.g., anti-BAFF-R antibody or fragment thereof, to BAFF-R, such as human BAFF-R, is at or about 10 nM, 20 nM, 30 nM, 40 nM, 50 nM, 60 nM, 70 nM, 80 nM, 90 nM or 100 nM, or a range defined by any of the foregoing. In certain embodiments, the EC₅₀ and/or the K_{D} of the binding molecule, e.g., anti-BAFF-R antibody or fragment thereof, to BAFF-R, such as human BAFF-R, is at or about 10 nM, 20 nM, 30 nM, 40 nM, 50 nM, 60 nM, 70 nM, 80 nM, 90 nM or 100 nM, or a range defined by any of the foregoing. In certain embodiments, the EC₅₀ and/or the K_{D} of the binding molecule, e.g., anti-BAFF-R antibody or fragment thereof, to BAFF-R, such as human BAFF-R, is at or about 40 nM, 41 nM, 42 nM, 43 nM, 44 nM, or 45 nM, or a range defined by any of the foregoing.

In some embodiments, the provided binding molecule, e.g., anti-BAFF-R antibody or antigen-binding fragment thereof or receptors containing such antibody or antigen-binding fragments, has a fast off-rate (dissociation rate constant; k_{off} or k_{d}; units of l/s or s⁻¹). In some embodiments, the off-rate (k_{off} or k_{d}) of the provided binding molecules is between at or about 1×10⁻⁵ s⁻¹ and at or about 1×10⁻² s⁻¹, such as at or about 5×10⁻⁵ s⁻¹ and at or about 9×10⁻³ s⁻¹, at or about 1×10⁻⁴ s⁻¹ and at or about 8×10⁻³ s⁻¹, at or about 5×10⁻⁴ s⁻¹ and at or about 7×10⁻³ s⁻¹, at or about 1×10⁻³ s⁻¹ and at or about 6×10⁻³ s⁻¹, and at or about 4×10⁻³ s⁻¹ and at or about 6×10⁻³ s⁻¹. In some embodiments, the off-rate (k_{off} or k_{d}) of the provided binding molecules is at least at or about 1×10⁻⁵ s⁻¹, 5×10⁻⁵ s⁻¹, 1×10⁻⁴ s⁻¹, 5×10⁻⁴ s⁻¹, 1×10⁻³ s⁻¹, 5×10⁻³ s⁻¹, or 1×10⁻² s⁻¹. In some embodiments, the off-rate (k_{off} or k_{d}) of the provided binding molecules is at least at or about 6×10⁻⁴ s⁻¹, 7×10⁻⁴ s⁻¹, 8×10⁻⁴ s⁻¹, 9×10⁻⁴ s⁻¹, 1×10⁻³ s⁻¹, 2×10⁻³ s⁻¹, 3×10⁻³ s⁻¹, 4×10⁻³ s⁻¹, 5×10⁻³ s⁻¹, 6×10⁻³ s⁻¹, 7×10⁻³ s⁻¹, 8×10⁻³ s⁻¹, 9×10⁻³ s⁻¹ or 1×10⁻² s⁻¹. In some embodiments, the off-rate (k_{off} or k_{d}) of the provided binding molecules is at least at or about 4×10⁻³ s⁻¹, 5×10⁻³ s⁻¹ or 6×10⁻³ s⁻¹, or a range defined by any of the foregoing. In some embodiments, the provided binding molecule, e.g., anti-BAFF-R antibody or antigen-binding fragment thereof or receptors containing such antibody or antigen-binding fragments, has an off-rate that is at least at or about 2-fold, 3-fold, 4-fold, 5-fold, 6-fold, 7-fold, 8-fold, 9-fold or 10-fold faster than the off-rate of a reference anti-BAFF-R antibody or an antigen-binding fragment thereof, or receptors containing such antibody or antigen-binding fragments.

In some embodiments, the binding affinity of a binding molecule, such as an anti-BAFF-R antibody, for different antigens, e.g., BAFF-R from different species can be compared to determine the species cross-reactivity. For example, species cross-reactivity can be classified as high cross reactivity or low cross reactivity. In some embodiments, the equilibrium dissociation constant, K_{D}, for different antigens, e.g., BAFF-R from different species such as human, cynomolgus monkey or mouse, can be compared to determine species cross-reactivity. In some embodiments, the species cross-reactivity of an anti-BAFF-R antibody can be high, e.g., the anti-BAFF-R antibody binds to human BAFF-R and a species variant BAFF-R to a similar degree, e.g., the ratio of K_{D} for human BAFF-R and K_{D} for the species variant BAFF-R is or is about 1. In some embodiments, the species cross-reactivity of an anti-BAFF-R antibody can be low, e.g., the anti-BAFF-R antibody has a high affinity for human BAFF-R but a low affinity for a species variant BAFF-R, or vice versa. For example, the ratio of K_{D} for the species variant BAFF-R and K_{D} for the human BAFF-R is more than 10, 15, 20, 25, 30, 40, 50, 60, 70, 80, 90, 100, 200, 500, 1000, 2000 or more, and the anti-BAFF-R antibody has low species cross-reactivity. The degree of species cross-reactivity can be compared with the species cross-reactivity of a known antibody, such as a reference antibody.

In some embodiments, the provided antibodies or antigen binding fragments thereof bind to a similar degree to human BAFF-R and non-human BAFF-R. For example, in some embodiments, the provided antibodies or antigen binding fragments thereof bind to human BAFF-R, or an allelic variant or splice variant thereof, with a specific an equilibrium dissociation constant (K_{D}), and to non-human BAFF-R, such as a cynomolgus monkey BAFF-R, with a K_{D} that is similar, or about the same, or less than 2-fold different, or less than 5-fold different.

For example, in some embodiments, the provided antibodies or antigen binding fragments thereof bind to a human BAFF-R with a K_{D} of about or less than at or about 1 μM, 500 nM, 100 nM, 50 nM, 40 nM, 30 nM, 25 nM, 20 nM, 19 nM, 18 nM, 17 nM, 16 nM, 15 nM, 14 nM, 13 nM, 12 nM, 11 nM, 10 nM, 9 nM, 8 nM, 7 nM, 6 nM, 5 nM, 4 nM, 3 nM, 2 nM, or 1 nM or less, and binds to a cynomolgus monkey BAFF-R with a K_{D} of about or less than at or about 1μ, 500 nM, 100 nM, 50 nM, 40 nM, 30 nM, 25 nM, 20 nM, 19 nM, 18 nM, 17 nM, 16 nM, 15 nM, 14 nM, 13 nM, 12 nM, 11 nM, 10 nM, 9 nM, 8 nM, 7 nM, 6 nM, 5 nM, 4 nM, 3 nM, 2 nM, or 1 nM or less. In some embodiments, the provided antibodies or antigen binding fragments thereof bind to a mouse BAFF-R protein with a K_{D} of about or less than at or about 1 μM, 500 nM, 100 nM, 50 nM, 40 nM, 30 nM, 25 nM, 20 nM, 19 nM, 18 nM, 17 nM, 16 nM, 15 nM, 14 nM, 13 nM, 12 nM, 11 nM, 10 nM, 9 nM, 8 nM, 7 nM, 6 nM, 5 nM, 4 nM, 3 nM, 2 nM, or 1 nM or less. In some embodiments, the provided antibodies or antigen binding fragments thereof bind to a human BAFF-R, a cynomolgus monkey BAFF-R and a mouse BAFF-R with high affinity. In some embodiments, the provided antibodies or antigen binding fragments thereof bind to a human BAFF-R and cynomolgus monkey BAFF-R with a high affinity, and to a mouse BAFF-R with low affinity. In some embodiments, the provided antibodies or antigen binding fragments thereof bind to a human BAFF-R and BAFF-R from other species, or other variants of the BAFF-R protein, with high affinity.

In some embodiments, the total binding capacity (Rₘₐₓ), as measured using particular surface plasmon resonance (SPR) conditions, is used to determine the ability or capacity of binding of the provided antibody or antigen binding fragment thereof, to the antigen, e.g., BAFF-R, such as human BAFF-R. For SPR analysis, the “ligand” is the immobilized target molecule on the surface of the sensor, for example, BAFF-R, and the “analyte” is the tested molecule, e.g., antibody, for binding to the “ligand”. For example, the “analyte” can be any of the provided antibodies or antigen binding fragments thereof, that binds to BAFF-R. For a particular ligand and analyte pair in SPR, the Rₘₐₓ can be determined assuming a 1:1 binding stoichiometry model, for a particular condition. In some embodiments, binding capacity (Rₘₐₓ) can be determined using the following formula: Rₘₐₓ (RU)=(analyte molecular weight)/(ligand molecular weight) x immobilized ligand level (RU). In particular aspects of SPR conditions, the Rₘₐₓ of binding between any of the provided antibody or antigen binding fragment thereof and BAFF-R, such as human BAFF-R or a cynomolgus BAFF-R, is at least or at least about 50 resonance units (RU), such as about 25 RU, 20 RU, 15 RU, 10 RU, 5 RU or 1 RU.

In some embodiments, properties or features of the provided antibodies are described in relation to properties observed for another antibody, e.g., a reference antibody. In some embodiments, the reference antibody is a non-human anti-BAFF-R antibody, such as a rabbit or chimeric or humanized anti-BAFF-R antibody. In some embodiments, the provided antibody contains heavy and light chain CDRs that are distinct from the CDRs present in the reference antibody or antibodies. Among the provided antibodies are those that compete for binding with and/or bind to the same or overlapping epitopes of BAFF-R as those bound by a reference antibody or antibody but nonetheless contain distinct CDRs, e.g., distinct heavy and/or light chain CDR1, CDR2, and CDR3.

In some embodiments, the antibody has an affinity that is about the same as or lower than that of the corresponding form of the reference antibody, e.g., EC₅₀ or K_{D} that is no more than at or about 1.5-fold or no more than at or about 2-fold greater, no more than at or about 3-fold greater, and/or no more than at or about 10-fold greater, than the EC₅₀ or K_{D} of the corresponding form of the reference antibody. In some embodiments, the antibody has an affinity that is about the same as or lower than that of the corresponding form of the reference antibody, e.g., EC₅₀ or K_{D} that is at least at or about 1.5-fold greater, at least at or about 2-fold greater, at least at or about 3-fold greater, at least at or about 5-fold greater, at least at or about 10-fold greater, at least at or about 20-fold greater, at least at or about 25-fold greater, at least at or about 30-fold greater, at least at or about 40-fold greater, at least at or about 50-fold greater, or at least at or about 100-fold greater, than the EC₅₀ or K_{D} of the corresponding form of the reference antibody. In some embodiments, the antibody has an affinity that is about the same as or lower than that of the corresponding form of the reference antibody, an affinity that is at or about 1.5-fold, 2-fold, 3-fold, 4-fold, 5-fold, 6-fold, 7-fold, 8-fold, 9-fold, 10-fold, 20-fold, 25-fold, 30-fold, 40-fold, 50-fold or 100-fold lower than the affinity of the reference antibody.

In some embodiments, the antibody has an affinity that is greater than that of the corresponding form of the reference antibody, e.g., EC₅₀ or K_{D} that is lower than or lower than at or about 5-fold, 10-fold, 20-fold, 30-fold, 40-fold, 50-fold, 60-fold, 70-fold, 80-fold, 90-fold, 100-fold, 150-fold, 200-fold, 250-fold or lower than the EC₅₀ or K_{D} of the corresponding form of the reference antibody.

In some embodiments, the antibodies display a binding preference for BAFF-R-expressing cells as compared to BAFF-R-negative cells, such as particular cells known and/or described herein to express BAFF-R and known not to express BAFF-R. In some embodiments, the binding preference is observed where a significantly greater degree of binding is measured to the BAFF-R-expressing, as compared to the non-expressing, cells or cells expressing a related but different antigen. In some embodiments, the fold change in degree of binding detected, for example, as measured by mean fluorescence intensity in a flow cytometry-based assay and/or dissociation constant or EC₅₀, to the BAFF-R-expressing cells as compared to the non-BAFF-R-expressing cells or cells expressing a related but different antigen, is at least at or about 1.5, 2, 3, 4, 5, 6, or more, and/or is about as great, about the same, at least as great or at least about as great, or greater, than the fold change observed for the corresponding form of the reference antibody. In some cases, the total degree of observed binding to BAFF-R or to the BAFF-R-expressing cells is approximately the same, at least as great, or greater than that observed for the corresponding form of the reference antibody.

In some aspects, the affinity is at or about the same degree or substantially the same degree of affinity compared to the corresponding form of the reference antibody, such as rabbit BAFF-R antibody. In some aspects, the affinity is at least at or about 80, 85, 90, 95, or 99% of or the same as that of the corresponding form of the reference antibody.

In some embodiments, the antibody specifically binds to an epitope that overlaps with the epitope of BAFF-R bound by a reference antibody. In some aspects, among such antibodies are antibodies that bind to the same or a similar epitope as the reference antibody. In some embodiments, the antibodies bind to the same or a similar epitope or an epitope within the same region or containing residues within the same region of BAFF-R as a reference antibody. In some embodiments, the antibody inhibits binding to and/or competes for binding to BAFF-R, such as human BAFF-R, with the reference antibody.

Competitive inhibition assays are known and include ELISA-based, flow cytometry-based assays, and RIA-based assays. In some aspects, competitive inhibition assays are carried out by incorporating an excess of an unlabeled form of one of the antibodies and assessing its ability to block binding of the other antibody, which is labeled with a detectable marker, such that degree of binding and reduction thereof can be assessed by detection of the label or marker. In some embodiments, addition of the provided antibody in excess, e.g., 1-, 2-, 5-, 10-, 50- or 100-fold excess, as compared to the amount or concentration of the reference antibody, inhibits binding to the antigen by the reference antibody (or vice versa). In some embodiments, the inhibition of binding is by at least 50%, and in some embodiments by at least 75%, 90% or 99%. In some aspects, the competitive inhibition is as measured in a competitive binding assay (see, e.g., Junghans et al., Cancer Res. 1990:50:1495-1502). Competition assays may be used to identify an antibody that competes with any of the antibodies described herein. Assays for mapping epitopes bound by the antibodies and reference antibodies also may be used and are known.

Anti-BAFF-R antibodies provided herein may be identified, screened for, or characterized for their physical/chemical properties and/or biological activities by various known assays. In one aspect, the antibody is tested for its antigen binding activity, e.g., by known methods such as ELISA, Western blotting, and/or flow cytometric assays, including cell-based binding assays, for example, assessing binding of the antibody (e.g., conjugated to a fluorescent marker or tagged) to a cell expressing the target antigen, e.g., BAFF-R, in some cases compared to results using cells that do not express the target antigen, e.g., BAFF-R. Binding affinity may be measured as K_{D}, K_{A} or EC₅₀.

### D. Variants

In certain embodiments, the antibodies include one or more amino acid variations, e.g., substitutions, deletions, insertions, and/or mutations, compared to the sequence of an antibody described herein. Exemplary variants include those designed to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

In certain embodiments, the antibodies include one or more amino acid substitutions, e.g., as compared to an antibody sequence described herein and/or compared to a sequence of a natural repertoire, e.g., human repertoire. Sites of interest for substitutional mutagenesis include the CDRs and FRs. Amino acid substitutions may be introduced into an antibody of interest and the products screened for a desired activity, e.g., retained/improved antigen binding, decreased immunogenicity, improved half-life, and/or improved effector function, such as the ability to promote antibody-dependent cellular cytotoxicity (ADCC) or complement-dependent cytotoxicity (CDC).

In some embodiments, one or more residues within a CDR of a parent antibody (e.g. a humanized or human antibody) is/are substituted. In some embodiments, the substitution is made to revert a sequence or position in the sequence to a germline sequence, such as an antibody sequence found in the germline (e.g., human germline), for example, to reduce the likelihood of immunogenicity, e.g., upon administration to a human subject.

In some embodiments, alterations are made in CDR “hotspots,” residues encoded by codons that undergo mutation at high frequency during the somatic maturation process (see, e.g., Chowdhury, Methods Mol. Biol. 207:179-196 (2008)), and/or residues that contact antigen, with the resulting variant V_{H} or V_{L} being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001)). In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves CDR-directed approaches, in which several CDR residues (e.g., 4-6 residues at a time) are randomized. CDR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more CDRs so long as such alterations do not substantially reduce the ability of the antibody to bind antigen. For example, conservative alterations (e.g., conservative substitutions as provided herein) that do not substantially reduce binding affinity may be made in CDRs. Such alterations may, for example, be outside of antigen contacting residues in the CDRs. In certain embodiments of the variant V_{H} and V_{L} sequences provided above, each CDR either is unaltered, or contains no more than one, two or three amino acid substitutions.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme or a polypeptide which increases the serum half-life of the antibody.

### II. RECOMBINANT RECEPTORS (E.G., CHIMERIC ANTIGEN RECEPTORS)

Provided in some aspects are BAFF-R-binding agents, such as recombinant receptors or chimeric antigen receptors (CARs) comprising extracellular binding domains that bind to BAFF-R. In some embodiments, the extracellular binding domain is a bispecific binding domain that binds to BAFF-R and CD19. The provided recombinant receptors generally contain the extracellular binding domain and an intracellular signaling domain. Among the provided receptors are recombinant receptors in which the extracellular binding domain is composed of antibodies or antigen-binding fragments thereof, such as an anti-BAFF-R antibody or an anti-BAFF-R antibody and an anti-CD19 antibody. Such receptors include chimeric antigen receptors that contain such antibodies. In some embodiments, the recombinant receptor, such as a CAR, contains an extracellular binding domain, a transmembrane domain, and an intracellular signaling domain. In some embodiments, the extracellular binding domain of the recombinant receptor, such as a CAR, targets BAFF-R. In some embodiments, the extracellular binding domain of the recombinant receptor, such as a CAR, targets both BAFF-R and CD19. In some embodiments, the recombinant receptor, such as a CAR, also includes a spacer domain (e.g. hinge domain) separating the extracellular binding domain and the transmembrane domain.

In some embodiments, the extracellular binding domain of any of the provided recombinant receptors, such as a CAR, includes a BAFF-R-binding domain. In some embodiments, the BAFF-R binding domain includes cell surface proteins containing antibodies (e.g., antigen-binding antibody fragments) and/or other binding peptides that specifically bind to BAFF-R (e.g., human BAFF-R protein). In some aspects, the binding domain binds to an extracellular portion of BAFF-R. In some aspects, the BAFF-R-binding domain binds to an extracellular portion of BAFF-R. In some examples, the recombinant receptors are chimeric antigen receptors, such as those containing anti-BAFF-R antibodies or antigen-binding fragments thereof.

Also provided herein are recombinant receptors, such as chimeric antigen receptors (CARs), in which the extracellular binding domains binds to both BAFF-R and CD19. In some embodiments, the extracellular binding domains comprise a BAFF-R-binding domain that binds to BAFF-R and a CD19-binding domain that binds to CD19. The BAFF-R-binding domain includes cell surface proteins containing antibodies (e.g., antigen-binding antibody fragments) and/or other binding peptides that specifically bind to BAFF-R (e.g., human BAFF-R protein). The CD19-binding domain includes cell surface proteins containing antibodies (e.g., antigen-binding antibody fragments) and/or other binding peptides that specifically bind to CD19 (e.g., human CD19). In some aspects, the binding domain binds to an extracellular portion of BAFF-R. In some aspects, the BAFF-R-binding domain binds to an extracellular portion of BAFF-R. In some aspects, the binding domain binds to an extracellular portion of CD19. In some aspects, the CD19-binding domain binds to an extracellular portion of CD19.

Also provided herein are polynucleotides encoding any of the provided recombinant receptors, such as any of the provided CARs.

In some embodiments, among the provided polynucleotides are those that encode recombinant receptors, such as a CAR, that specifically bind BAFF-R. The provided polynucleotides can be incorporated into constructs, such as deoxyribonucleic acid (DNA) or RNA constructs, such as those that can be introduced into cells for expression of the encoded recombinant BAFF-R-binding domains. Hence, also provided herein are engineered cells containing any of the provided BAFF-R targeted binding receptors. Exemplary engineered cells and methods of preparing same are described in Section III. Also provided herein are compositions and articles of manufacture and uses of any of the engineered cells.

In some embodiments, among the provided polynucleotides are those that encode recombinant receptors, such as a CAR, that specifically bind BAFF-R and CD19 (BAFF-R/CD19). The provided polynucleotides can be incorporated into constructs, such as deoxyribonucleic acid (DNA) or RNA constructs, such as those that can be introduced into cells for expression of the encoded recombinant BAFF-R/CD19-binding domains. Hence, also provided herein are engineered cells containing any of the provided BAFF-R/CD19 bispecific binding receptors. Exemplary engineered cells and methods of preparing same are described in Section III. Also provided herein are compositions and articles of manufacture and uses of any of the engineered cells.

Also provided are cells expressing the recombinant receptors and uses thereof in adoptive cell therapy, such as treatment of diseases and disorders associated with BAFF-R expression, CD19 expression, or both.

### A. Extracellular Binding Domains

### 1. Baff-R Binding Domain

In some embodiments, the extracellular binding domain of a provided recombinant receptor, such as a CAR, includes, is, or comprises an anti-BAFF-R antibody. Thus, the chimeric receptors, e.g., CARs, typically include in their extracellular portions a BAFF-R-binding domain, such as antigen-binding fragments, domains, or portions, or one or more antibody variable regions, and/or antibody molecules, such as those described herein.

In some embodiments, the extracellular binding domain comprises a BAFF-R-binding domain. In some embodiments, the BAFF-R-binding domain comprises an anti-BAFF-R antibody or antigen-binding fragment thereof. In some embodiments, the BAFF-R binding domain comprises a heavy chain variable (V_{H}) region and a light chain variable (V_{L}) region.

In some embodiments, the CAR includes a BAFF-R-binding domain comprising an antibody, such as a heavy chain variable (V_{H}) region and/or light chain variable (V_{L}) region of the antibody. In some embodiments, the (V_{H}) region and the (V_{L}) region of the BAFF-R-binding domain are joined by a linker. In some embodiments, the (V_{H}) region and the (V_{L}) region of the BAFF-R-binding domain comprise an scFv antibody fragment. In some embodiments, the provided BAFF-R-binding CARs contain an antibody, such as an anti-BAFF-R antibody, or an antigen-binding fragment thereof that confers the BAFF-R-binding properties of the provided CAR. In some embodiments, the CAR contains a variable heavy (V_{H}) and/or a variable light (V_{L}) region derived from an antibody described Section I.

In some embodiments, the antibody, e.g., the anti-BAFF-R antibody, or antigen-binding fragment, contains a heavy and/or light chain variable (V_{H} or V_{L}) region sequence as described, or a sufficient antigen-binding portion thereof. In some embodiments, the anti-BAFF-R antibody, e.g., antigen-binding fragment, contains a V_{H} region sequence or sufficient antigen-binding portion thereof that contains a CDR-H1, CDR-H2 and/or CDR-H3 as described. In some embodiments, the anti-BAFF-R antibody, e.g., antigen-binding fragment, contains a V_{L} region sequence or sufficient antigen-binding portion that contains a CDR-L1, CDR-L2 and/or CDR-L3 as described. In some embodiments, the anti-BAFF-R antibody, e.g., antigen-binding fragment, contains a V_{H} region sequence that contains a CDR-H1, CDR-H2 and/or CDR-H3 as described and contains a V_{L} region sequence that contains a CDR-L1, CDR-L2 and/or CDR-L3 as described. Also among the antibodies are those having sequences at least at or about 90%, at or about 91%, at or about 92%, at or about 93%, at or about 94%, at or about 95%, at or about 96%, at or about 97%, at or about 98%, or at or about 99% identical to such a sequence. In some embodiments, the antibody or antibody fragment, in the provided CAR, has a V_{H} region of any of the antibodies described in Section I.

In some embodiments, the antibody or antigen-binding fragment thereof, in the provided CAR, is a single-chain antibody fragment, such as a single chain variable fragment (scFv) or a diabody or a single domain antibody (sdAb). In some embodiments, the antibody or antigen-binding fragment is a single domain antibody comprising only the V_{H} region. In some embodiments, the antibody or antigen binding fragment is an scFv comprising a heavy chain variable (V_{H}) region and a light chain variable (V_{L}) region. In some embodiments, the single-chain antibody fragment (e.g., scFv) includes one or more linkers joining two antibody domains or regions, such as a heavy chain variable (V_{H}) region and a light chain variable (V_{L}) region. The linker typically is a peptide linker, e.g., a flexible and/or soluble peptide linker. Among the linkers are those rich in glycine and serine and/or in some cases threonine. In some embodiments, the linkers further include charged residues such as lysine and/or glutamate, which can improve solubility. In some embodiments, the linkers further include one or more proline.

Accordingly, the provided CARs contain anti-BAFF-R antibodies that include single-chain antibody fragments, such as scFvs and diabodies, particularly human single-chain antibody fragments, typically comprising linker(s) joining two antibody domains or regions, such V_{H} and V_{L} regions. In some aspects, a BAFF-R scFv provided herein comprises the amino acid sequence set forth in SEQ ID NOs: 11-15, or has an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NOs: 11-15.

The linker typically is a peptide linker, e.g., a flexible and/or soluble peptide linker, such as one rich in glycine and serine. In some embodiments, the BAFF-R-binding domain comprises a linker between the V_{H} and V_{L} regions. In some embodiments, in order from N- to C-terminus, the BAFF-R-binding domain comprises one of the V_{H} and V_{L} regions, a linker, and the other of the V_{H} and V_{L} regions. In some embodiments, the linker is set forth in SEQ ID NO: 58. Thus, in some embodiments, in order from N- to C-terminus, the BAFF-R-binding domain comprises one of the V_{H} and V_{L} regions, the linker set forth in SEQ ID NO: 58, and the other of the V_{H} and V_{L} regions.

In some aspects, the linkers rich in glycine and serine (and/or threonine) include at least 80%, 85%, 90%, 91%, 92%, 93%, 94%, 95%, 96%, 97%, 98%, or 99% such amino acid(s). In some embodiments, they include at least at or about 50%, 55%, 60%, 70%, or 75%, glycine, serine, and/or threonine. In some embodiments, the linker is comprised substantially entirely of glycine, serine, and/or threonine. The linkers generally are between about 5 and about 50 amino acids in length, typically between at or about 10 and at or about 30, e.g., 10, 11, 12, 13, 14, 15, 16, 17, 18, 19, 20, 21, 22, 23, 24, 25, 26, 27, 28, 29, or 30, and in some examples between 10 and 25 amino acids in length. Exemplary linkers include linkers having various numbers of repeats of the sequence GGGGS (4GS; SEQ ID NO: 60), such as between 2, 3, 4 and 5 repeats of such a sequence. Exemplary linkers include those having or consisting of an sequence set forth in SEQ ID NO: 58 (GGGGSGGGGSGGGGS). Exemplary linkers further include those having or consisting of the sequence set forth in SEQ ID NO: 59 (GSTSGSGKPGSGEGSTKG), SEQ ID NO: 61 (GGGGSGGGGS), and SEQ ID NO: 62 (GGGGSGGGGSGGGGSGGGGS).

Accordingly, in some embodiments, the provided embodiments include single-chain antibody fragments, e.g., scFvs, comprising one or more of the aforementioned linkers, such as glycine/serine rich linkers, including linkers having repeats of GGGS (SEQ ID NO: 60), such as the linker set forth in SEQ ID NO: 58, 61, or 62.

### 2. Bispecific BAFF-R/CD19 Binding Domain

In some embodiments, the extracellular binding domain of a provided recombinant receptor, such as a CAR, includes, is, or comprises an anti-BAFF-R antibody and an anti-CD19 antibody. Thus, the chimeric receptors, e.g., CARs, typically include in their extracellular portions a BAFF-R-binding domain and a CD19-binding domain, such as antigen-binding fragments, domains, or portions, or one or more antibody variable regions, and/or antibody molecules, such as those described herein.

In some embodiments, the extracellular binding domain comprises a BAFF-R-binding domain and a CD19-binding domain. In some embodiments, the BAFF-R-binding domain comprises an anti-BAFF-R antibody or antigen-binding fragment thereof. In some embodiments, the CD19-binding domain comprises an anti-CD19 antibody of antigen-binding fragment thereof. In some embodiments, the BAFF-R binding domain comprises a heavy chain variable (V_{H}) region and a light chain variable (V_{L}) region. In some embodiments, the CD19-binding domain comprises a heavy chain variable (V_{H}) region and a light chain variable (V_{L}) region.

a. BAFF-R-Binding Domain

In some embodiments, the CAR includes a BAFF-R-binding domain comprising an antibody, such as a heavy chain variable (V_{H}) region and/or light chain variable (V_{L}) region of the antibody. In some embodiments, the (V_{H}) region and the (V_{L}) region of the BAFF-R-binding domain are joined by a linker. In some embodiments, the (V_{H}) region and the (V_{L}) region of the BAFF-R-binding domain comprise an scFv antibody fragment. In some embodiments, the provided BAFF-R-binding CARs contain an antibody, such as an anti-BAFF-R antibody, or an antigen-binding fragment thereof that confers the BAFF-R-binding properties of the provided CAR. In some embodiments, the CAR contains a variable heavy (V_{H}) and/or a variable light (V_{L}) region derived from an antibody described Section I.

In some embodiments, the antibody, e.g., the anti-BAFF-R antibody, or antigen-binding fragment, contains a heavy and/or light chain variable (V_{H} or V_{L}) region sequence as described, or a sufficient antigen-binding portion thereof. In some embodiments, the anti-BAFF-R antibody, e.g., antigen-binding fragment, contains a V_{H} region sequence or sufficient antigen-binding portion thereof that contains a CDR-H1, CDR-H2 and/or CDR-H3 as described. In some embodiments, the anti-BAFF-R antibody, e.g., antigen-binding fragment, contains a V_{L} region sequence or sufficient antigen-binding portion that contains a CDR-L1, CDR-L2 and/or CDR-L3 as described. In some embodiments, the anti-BAFF-R antibody, e.g., antigen-binding fragment, contains a V_{H} region sequence that contains a CDR-H1, CDR-H2 and/or CDR-H3 as described and contains a V_{L} region sequence that contains a CDR-L1, CDR-L2 and/or CDR-L3 as described. Also among the antibodies are those having sequences at least at or about 90%, at or about 91%, at or about 92%, at or about 93%, at or about 94%, at or about 95%, at or about 96%, at or about 97%, at or about 98%, or at or about 99% identical to such a sequence. In some embodiments, the antibody or antibody fragment, in the provided CAR, has a V_{H} region of any of the antibodies described in Section I.

In some embodiments, the CAR contains an antibody or antigen-binding fragment thereof, that has a heavy chain variable (V_{H}) region having the amino acid sequence set forth in SEQ ID NO: 1, or an amino acid sequence that has at least at or about 90%, at or about 91%, at or about 92%, at or about 93%, at or about 94%, at or about 95%, at or about 96%, at or about 97%, at or about 98%, or at or about 99% sequence identity to the V_{H} region amino acid set forth in SEQ ID NO: 1, or contains a CDR-H1, CDR-H2, and/or CDR-H3 present in such a V_{H} sequence.

In some embodiments, the CAR contains an antibody or antigen-binding fragment thereof, that has a heavy chain variable (V_{H}) region having the amino acid sequence set forth in SEQ ID NO: 3, or an amino acid sequence that has at least at or about 90%, at or about 91%, at or about 92%, at or about 93%, at or about 94%, at or about 95%, at or about 96%, at or about 97%, at or about 98%, or at or about 99% sequence identity to the V_{H} region amino acid set forth in SEQ ID NO: 3, or contains a CDR-H1, CDR-H2, and/or CDR-H3 present in such a V_{H} sequence.

In some embodiments, the CAR contains an antibody or antigen-binding fragment thereof, that has a heavy chain variable (V_{H}) region having the amino acid sequence set forth in SEQ ID NO: 5, or an amino acid sequence that has at least at or about 90%, at or about 91%, at or about 92%, at or about 93%, at or about 94%, at or about 95%, at or about 96%, at or about 97%, at or about 98%, or at or about 99% sequence identity to the V_{H} region amino acid set forth in SEQ ID NO: 5, or contains a CDR-H1, CDR-H2, and/or CDR-H3 present in such a V_{H} sequence.

In some embodiments, the CAR contains an antibody or antigen-binding fragment thereof, that has a heavy chain variable (V_{H}) region having the amino acid sequence set forth in SEQ ID NO: 7, or an amino acid sequence that has at least at or about 90%, at or about 91%, at or about 92%, at or about 93%, at or about 94%, at or about 95%, at or about 96%, at or about 97%, at or about 98%, or at or about 99% sequence identity to the V_{H} region amino acid set forth in SEQ ID NO: 7, or contains a CDR-H1, CDR-H2, and/or CDR-H3 present in such a V_{H} sequence.

In some embodiments, the CAR contains an antibody or antigen-binding fragment thereof, that has a heavy chain variable (V_{H}) region having the amino acid sequence set forth in SEQ ID NO: 9, or an amino acid sequence that has at least at or about 90%, at or about 91%, at or about 92%, at or about 93%, at or about 94%, at or about 95%, at or about 96%, at or about 97%, at or about 98%, or at or about 99% sequence identity to the V_{H} region amino acid set forth in SEQ ID NO: 9, or contains a CDR-H1, CDR-H2, and/or CDR-H3 present in such a V_{H} sequence.

In some embodiments, the V_{H} region of an antibody or antigen-binding fragment thereof comprises a CDR-H1, CDR-H2, and/or CDR-H3 according to Kabat numbering. In some embodiments, the V_{H} region of an antibody or antigen-binding fragment thereof comprises a CDR-H1, CDR-H2, and/or CDR-H3 according to Chothia numbering. In some embodiments, the V_{H} region of an antibody or antigen-binding fragment thereof comprises a CDR-H1, CDR-H2, and/or CDR-H3 according to AbM numbering.

In some embodiments, the CAR contains an antibody or antigen-binding fragment thereof, that has a variable heavy chain (V_{H}) region comprising a CDR-H1 comprising the amino acid sequence set forth in SEQ ID NO: 16, a CDR-H2 comprising the amino acid sequence set forth in SEQ ID NO: 17, and a CDR-H3 comprising the amino acid sequence set forth in SEQ ID NO:18. In some embodiments, the antibody or antigen-binding fragment thereof comprises a V_{H} region comprising a CDR-H1, a CDR-H2, and a CDR-H3 comprising the amino acid sequence set forth in SEQ ID NO: 16, 17, and 18, respectively. In some embodiments, the antibody or antigen-binding fragment thereof comprises a V_{H} region comprising the amino acid sequence set forth in SEQ ID NO: 16, 17, and 18.

In some embodiments of the antibody or antigen-binding fragment thereof provided herein, the V_{H} region comprises any of the CDR-H1, CDR-H2 and CDR-H3 as described and comprises a framework region 1 (FR1), a FR2, a FR3 and/or a FR4 having at least at or about 90%, at or about 91%, at or about 92%, at or about 93%, at or about 94%, at or about 95%, at or about 96%, at or about 97%, at or about 98%, or at or about 99% sequence identity, respectively, to a FR1, a FR2, a FR3 and/or a FR4 contained within the V_{H} region amino acid sequence set forth in SEQ ID NO: 1. In some embodiments, the antibody or antigen-binding fragment thereof comprises a V_{H} region comprising the amino acid sequence set forth in SEQ ID NO: 1.

In some embodiments, the CAR contains an antibody or antigen-binding fragment thereof, that has a variable heavy chain (V_{H}) region comprising a CDR-H1 comprising the amino acid sequence set forth in SEQ ID NO: 22, a CDR-H2 comprising the amino acid sequence set forth in SEQ ID NO: 23, and a CDR-H3 comprising the amino acid sequence set forth in SEQ ID NO: 24. In some embodiments, the antibody or antigen-binding fragment thereof comprises a V_{H} region comprising a CDR-H1, a CDR-H2, and a CDR-H3 comprising the amino acid sequence set forth in SEQ ID NO: 22, 23, and 24, respectively. In some embodiments, the antibody or antigen-binding fragment thereof comprises a V_{H} region comprising the amino acid sequence set forth in SEQ ID NO: 22, 23, and 24.

In some embodiments of the antibody or antigen-binding fragment thereof provided herein, the V_{H} region comprises any of the CDR-H1, CDR-H2 and CDR-H3 as described and comprises a framework region 1 (FR1), a FR2, a FR3 and/or a FR4 having at least at or about 90%, at or about 91%, at or about 92%, at or about 93%, at or about 94%, at or about 95%, at or about 96%, at or about 97%, at or about 98%, or at or about 99% sequence identity, respectively, to a FR1, a FR2, a FR3 and/or a FR4 contained within the V_{H} region amino acid sequence set forth in SEQ ID NO: 3. In some embodiments, the antibody or antigen-binding fragment thereof comprises a V_{H} region comprising the amino acid sequence set forth in SEQ ID NO: 3.

In some embodiments, the CAR contains an antibody or antigen-binding fragment thereof, that has a variable heavy chain (V_{H}) region comprising a CDR-H1 comprising the amino acid sequence set forth in SEQ ID NO: 28, a CDR-H2 comprising the amino acid sequence set forth in SEQ ID NO: 29, and a CDR-H3 comprising the amino acid sequence set forth in SEQ ID NO: 30. In some embodiments, the antibody or antigen-binding fragment thereof comprises a V_{H} region comprising a CDR-H1, a CDR-H2, and a CDR-H3 comprising the amino acid sequence set forth in SEQ ID NO: 28, 29, and 30, respectively. In some embodiments, the antibody or antigen-binding fragment thereof comprises a V_{H} region comprising the amino acid sequence set forth in SEQ ID NO: 28, 29, and 30.

In some embodiments of the antibody or antigen-binding fragment thereof provided herein, the V_{H} region comprises any of the CDR-H1, CDR-H2 and CDR-H3 as described and comprises a framework region 1 (FR1), a FR2, a FR3 and/or a FR4 having at least at or about 90%, at or about 91%, at or about 92%, at or about 93%, at or about 94%, at or about 95%, at or about 96%, at or about 97%, at or about 98%, or at or about 99% sequence identity, respectively, to a FR1, a FR2, a FR3 and/or a FR4 contained within the V_{H} region amino acid sequence set forth in SEQ ID NO: 5. In some embodiments, the antibody or antigen-binding fragment thereof comprises a V_{H} region comprising the amino acid sequence set forth in SEQ ID NO: 5.

In some embodiments, the CAR contains an antibody or antigen-binding fragment thereof, that has a variable heavy chain (V_{H}) region comprising a CDR-H1 comprising the amino acid sequence set forth in SEQ ID NO: 22, a CDR-H2 comprising the amino acid sequence set forth in SEQ ID NO: 32, and a CDR-H3 comprising the amino acid sequence set forth in SEQ ID NO: 24. In some embodiments, the antibody or antigen-binding fragment thereof comprises a V_{H} region comprising a CDR-H1, a CDR-H2, and a CDR-H3 comprising the amino acid sequence set forth in SEQ ID NO: 22, 32, and 24, respectively. In some embodiments, the antibody or antigen-binding fragment thereof comprises a V_{H} region comprising the amino acid sequence set forth in SEQ ID NO: 22, 32, and 24.

In some embodiments of the antibody or antigen-binding fragment thereof provided herein, the V_{H} region comprises any of the CDR-H1, CDR-H2 and CDR-H3 as described and comprises a framework region 1 (FR1), a FR2, a FR3 and/or a FR4 having at least at or about 90%, at or about 91%, at or about 92%, at or about 93%, at or about 94%, at or about 95%, at or about 96%, at or about 97%, at or about 98%, or at or about 99% sequence identity, respectively, to a FR1, a FR2, a FR3 and/or a FR4 contained within the V_{H} region amino acid sequence set forth in SEQ ID NO: 7. In some embodiments, the antibody or antigen-binding fragment thereof comprises a V_{H} region comprising the amino acid sequence set forth in SEQ ID NO: 7.

In some embodiments, the CAR contains an antibody or antigen-binding fragment thereof, that has a variable heavy chain (V_{H}) region comprising a CDR-H1 comprising the amino acid sequence set forth in SEQ ID NO: 35, a CDR-H2 comprising the amino acid sequence set forth in SEQ ID NO: 36, and a CDR-H3 comprising the amino acid sequence set forth in SEQ ID NO: 37. In some embodiments, the antibody or antigen-binding fragment thereof comprises a V_{H} region comprising a CDR-H1, a CDR-H2, and a CDR-H3 comprising the amino acid sequence set forth in SEQ ID NO: 35, 36, and 37, respectively. In some embodiments, the antibody or antigen-binding fragment thereof comprises a V_{H} region comprising the amino acid sequence set forth in SEQ ID NO: 35, 36, and 37.

In some embodiments of the antibody or antigen-binding fragment thereof provided herein, the V_{H} region comprises any of the CDR-H1, CDR-H2 and CDR-H3 as described and comprises a framework region 1 (FR1), a FR2, a FR3 and/or a FR4 having at least at or about 90%, at or about 91%, at or about 92%, at or about 93%, at or about 94%, at or about 95%, at or about 96%, at or about 97%, at or about 98%, or at or about 99% sequence identity, respectively, to a FR1, a FR2, a FR3 and/or a FR4 contained within the V_{H} region amino acid sequence set forth in SEQ ID NO: 9. In some embodiments, the antibody or antigen-binding fragment thereof comprises a V_{H} region comprising the amino acid sequence set forth in SEQ ID NO: 9.

In some embodiments, the antibody or antibody fragment, in the provided CAR comprising a V_{H} region further comprises a light chain or a sufficient antigen binding portion thereof. For example, in some embodiments, the antibody or antigen-binding fragment thereof contains a V_{H} region and a V_{L} region, or a sufficient antigen-binding portion of a V_{H} and V_{L} region. In such embodiments, a V_{H} region sequence can be any of the above described V_{H} sequence. In some such embodiments, the antibody is an antigen-binding fragment, such as a Fab or an scFv. In some such embodiments, the antibody is a full-length antibody that also contains a constant region.

In some embodiments, a CAR provided herein, contains an antibody such as an anti-BAFF-R antibody, or antigen-binding fragment thereof that contains any of the above V_{H} region and contains a variable light chain region or a sufficient antigen binding portion thereof. For example, in some embodiments, the CAR contains an antibody or antigen-binding fragment thereof that contains a V_{H} region and a variable light chain (V_{L}) region, or a sufficient antigen-binding portion of a V_{H} and V_{L} region. In such embodiments, a V_{H} region sequence can be any of the above described V_{H} sequence. In some such embodiments, the antibody is an antigen-binding fragment, such as a Fab or an scFv. In some such embodiments, the antibody is a full-length antibody that also contains a constant region. In some embodiments, the antibody has a V_{L} region described in Section I.

In some embodiments, the CAR contains an antibody or antigen-binding fragment thereof, that has a light chain variable (V_{L}) region having the amino acid sequence set forth in SEQ ID NO: 2, or an amino acid sequence that has at least at or about 90%, at or about 91%, at or about 92%, at or about 93%, at or about 94%, at or about 95%, at or about 96%, at or about 97%, at or about 98%, or at or about 99% sequence identity to the V_{L} region amino acid set forth in SEQ ID NO: 2, or contains a CDR-L1, CDR-L2, and/or CDR-L3 present in such a V_{L} sequence.

In some embodiments, the CAR contains an antibody or antigen-binding fragment thereof, that has a light chain variable (V_{L}) region having the amino acid sequence set forth in SEQ ID NO: 4, or an amino acid sequence that has at least at or about 90%, at or about 91%, at or about 92%, at or about 93%, at or about 94%, at or about 95%, at or about 96%, at or about 97%, at or about 98%, or at or about 99% sequence identity to the V_{L} region amino acid set forth in SEQ ID NO: 4, or contains a CDR-L1, CDR-L2, and/or CDR-L3 present in such a V_{L} sequence.

In some embodiments, the CAR contains an antibody or antigen-binding fragment thereof, that has a light chain variable (V_{L}) region having the amino acid sequence set forth in SEQ ID NO: 6, or an amino acid sequence that has at least at or about 90%, at or about 91%, at or about 92%, at or about 93%, at or about 94%, at or about 95%, at or about 96%, at or about 97%, at or about 98%, or at or about 99% sequence identity to the V_{L} region amino acid set forth in SEQ ID NO: 6, or contains a CDR-L1, CDR-L2, and/or CDR-L3 present in such a V_{L} sequence.

In some embodiments, the CAR contains an antibody or antigen-binding fragment thereof, that has a light chain variable (V_{L}) region having the amino acid sequence set forth in SEQ ID NO: 8, or an amino acid sequence that has at least at or about 90%, at or about 91%, at or about 92%, at or about 93%, at or about 94%, at or about 95%, at or about 96%, at or about 97%, at or about 98%, or at or about 99% sequence identity to the V_{L} region amino acid set forth in SEQ ID NO: 8, or contains a CDR-L1, CDR-L2, and/or CDR-L3 present in such a V_{L} sequence.

In some embodiments, the CAR contains an antibody or antigen-binding fragment thereof, that has a light chain variable (V_{L}) region having the amino acid sequence set forth in SEQ ID NO: 10, or an amino acid sequence that has at least at or about 90%, at or about 91%, at or about 92%, at or about 93%, at or about 94%, at or about 95%, at or about 96%, at or about 97%, at or about 98%, or at or about 99% sequence identity to the V_{L} region amino acid set forth in SEQ ID NO: 10, or contains a CDR-L1, CDR-L2, and/or CDR-L3 present in such a V_{L} sequence.

In some embodiments, the V_{L} region of an antibody or antigen-binding fragment thereof comprises a CDR-L1, CDR-L2, and/or CDR-L3 according to Kabat numbering. In some embodiments, the V_{L} region of an antibody or antigen-binding fragment thereof comprises a CDR-L1, CDR-L2, and/or CDR-L3 according to Chothia numbering. In some embodiments, the V_{L} region of an antibody or antigen-binding fragment thereof comprises a CDR-L1, CDR-L2, and/or CDR-L3 according to AbM numbering.

In some embodiments, the CAR contains an antibody or antigen-binding fragment thereof, that has a variable light chain (V_{L}) region comprising a CDR-L1 comprising the amino acid sequence set forth in SEQ ID NO: 19, a CDR-L2 comprising the amino acid sequence set forth in SEQ ID NO: 20, and a CDR-L3 comprising the amino acid sequence set forth in SEQ ID NO: 21. In some embodiments, the antibody or antigen-binding fragment thereof comprises a V_{L} region comprising a CDR-H1, a CDR-H2, and a CDR-H3 comprising the amino acid sequence set forth in SEQ ID NO: 19, 20, and 21, respectively. In some embodiments, the antibody or antigen-binding fragment thereof comprises a V_{L} region comprising the amino acid sequence set forth in SEQ ID NO: 19, 20, and 21. In some embodiments, the antibody or antigen-binding fragment thereof contains a CDR-L1, CDR-L2, and CDR-L3, respectively, contained within the V_{L} region amino acid sequence set forth in SEQ ID NO: 2.

In some embodiments, the CAR contains an antibody or antigen-binding fragment thereof, that has a variable light chain (V_{L}) region comprising a CDR-L1 comprising the amino acid sequence set forth in SEQ ID NO: 25, a CDR-L2 comprising the amino acid sequence set forth in SEQ ID NO: 26, and a CDR-L3 comprising the amino acid sequence set forth in SEQ ID NO: 27. In some embodiments, the antibody or antigen-binding fragment thereof comprises a V_{L} region comprising a CDR-H1, a CDR-H2, and a CDR-H3 comprising the amino acid sequence set forth in SEQ ID NO: 25, 26, and 27, respectively. In some embodiments, the antibody or antigen-binding fragment thereof comprises a V_{L} region comprising the amino acid sequence set forth in SEQ ID NO: 25, 26, and 27. In some embodiments, the antibody or antigen-binding fragment thereof contains a CDR-L1, CDR-L2, and CDR-L3, respectively, contained within the V_{L} region amino acid sequence set forth in SEQ ID NO: 4.

In some embodiments, the CAR contains an antibody or antigen-binding fragment thereof, that has a variable light chain (V_{L}) region comprising a CDR-L1 comprising the amino acid sequence set forth in SEQ ID NO: 31, a CDR-L2 comprising the amino acid sequence set forth in SEQ ID NO: 26, and a CDR-L3 comprising the amino acid sequence set forth in SEQ ID NO: 27. In some embodiments, the antibody or antigen-binding fragment thereof comprises a V_{L} region comprising a CDR-H1, a CDR-H2, and a CDR-H3 comprising the amino acid sequence set forth in SEQ ID NO: 31, 26, and 27, respectively. In some embodiments, the antibody or antigen-binding fragment thereof comprises a V_{L} region comprising the amino acid sequence set forth in SEQ ID NO: 31, 26, and 27. In some embodiments, the antibody or antigen-binding fragment thereof contains a CDR-L1, CDR-L2, and CDR-L3, respectively, contained within the V_{L} region amino acid sequence set forth in SEQ ID NO: 6.

In some embodiments, the CAR contains an antibody or antigen-binding fragment thereof, that has a variable light chain (V_{L}) region comprising a CDR-L1 comprising the amino acid sequence set forth in SEQ ID NO: 33, a CDR-L2 comprising the amino acid sequence set forth in SEQ ID NO: 26, and a CDR-L3 comprising the amino acid sequence set forth in SEQ ID NO: 34. In some embodiments, the antibody or antigen-binding fragment thereof comprises a V_{L} region comprising a CDR-H1, a CDR-H2, and a CDR-H3 comprising the amino acid sequence set forth in SEQ ID NO: 33, 26, and 34, respectively. In some embodiments, the antibody or antigen-binding fragment thereof comprises a V_{L} region comprising the amino acid sequence set forth in SEQ ID NO: 33, 26, and 34. In some embodiments, the antibody or antigen-binding fragment thereof contains a CDR-L1, CDR-L2, and CDR-L3, respectively, contained within the V_{L} region amino acid sequence set forth in SEQ ID NO: 8.

In some embodiments, the CAR contains an antibody or antigen-binding fragment thereof, that has a variable light chain (V_{L}) region comprising a CDR-L1 comprising the amino acid sequence set forth in SEQ ID NO: 38, a CDR-L2 comprising the amino acid sequence set forth in SEQ ID NO: 39, and a CDR-L3 comprising the amino acid sequence set forth in SEQ ID NO: 40. In some embodiments, the antibody or antigen-binding fragment thereof comprises a V_{L} region comprising a CDR-H1, a CDR-H2, and a CDR-H3 comprising the amino acid sequence set forth in SEQ ID NO: 38, 39, and 40, respectively. In some embodiments, the antibody or antigen-binding fragment thereof comprises a V_{L} region comprising the amino acid sequence set forth in SEQ ID NO: 38, 39, and 40. In some embodiments, the antibody or antigen-binding fragment thereof contains a CDR-L1, CDR-L2, and CDR-L3, respectively, contained within the V_{L} region amino acid sequence set forth in SEQ ID NO: 10.

Among the CARs provided herein is a CAR in which the antibody, such as an anti-BAFF-R antibody, or antibody fragment, in the provided CAR, comprises a V_{H} region amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 1, and a V_{L} region comprising an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 2.

In some embodiments, the V_{H} region of the antibody or antigen-binding fragment thereof comprises a CDR-H1, a CDR-H2, a CDR-H3, respectively, comprising the amino acid sequences of CDR-H1, CDR-H2, and CDR-H3 contained within the V_{H} region amino acid sequence set forth in SEQ ID NO: 1; and comprises a CDR-L1, a CDR-L2, a CDR-L3, respectively, comprising the amino acid sequences of CDR-L1, CDR-L2, and CDR-L3, respectively contained within the V_{L} region amino acid sequence set forth in SEQ ID NO: 2.

In some embodiments, the V_{H} region of the antibody or antigen-binding fragment thereof comprise the amino acid sequence set forth in SEQ ID NO: 1, and the V_{L} region of the antibody or antigen-binding fragment comprises the amino acid sequence set forth in SEQ ID NO: 2. In some embodiments, the V_{H} and V_{L} regions of the antibody or antigen-binding fragment thereof comprise the amino acid sequences set forth in SEQ ID NO: 1 and 2, respectively, or any antibody or antigen-binding fragment thereof that has at least 90% sequence identity to any of the above V_{H} and V_{L}, such as at least 90%, 91%, 92%, 93%, 94%, 95%, 96%, 97%, 98%, or 99% sequence identity thereto.

For example, the V_{H} and V_{L} regions of the antibody or antigen-binding fragment thereof provided therein comprise the amino acid sequence set forth in SEQ ID NO: 1 and 2, respectively.

Among the CARs provided herein is a CAR in which the antibody, such as an anti-BAFF-R antibody, or antibody fragment, in the provided CAR, comprises a V_{H} region amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 3, and a V_{L} region comprising an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 4.

In some embodiments, the V_{H} region of the antibody or antigen-binding fragment thereof comprises a CDR-H1, a CDR-H2, a CDR-H3, respectively, comprising the amino acid sequences of CDR-H1, CDR-H2, and CDR-H3 contained within the V_{H} region amino acid sequence set forth in SEQ ID NO: 3; and comprises a CDR-L1, a CDR-L2, a CDR-L3, respectively, comprising the amino acid sequences of CDR-L1, CDR-L2, and CDR-L3, respectively contained within the V_{L} region amino acid sequence set forth in SEQ ID NO: 4.

In some embodiments, the V_{H} region of the antibody or antigen-binding fragment thereof comprise the amino acid sequence set forth in SEQ ID NO: 3, and the V_{L} region of the antibody or antigen-binding fragment comprises the amino acid sequence set forth in SEQ ID NO: 4. In some embodiments, the V_{H} and V_{L} regions of the antibody or antigen-binding fragment thereof comprise the amino acid sequences set forth in SEQ ID NO: 3 and 4, respectively, or any antibody or antigen-binding fragment thereof that has at least 90% sequence identity to any of the above V_{H} and V_{L}, such as at least 90%, 91%, 92%, 93%, 94%, 95%, 96%, 97%, 98%, or 99% sequence identity thereto.

For example, the V_{H} and V_{L} regions of the antibody or antigen-binding fragment thereof provided therein comprise the amino acid sequence set forth in SEQ ID NO: 3 and 4, respectively.

Among the CARs provided herein is a CAR in which the antibody, such as an anti-BAFF-R antibody, or antibody fragment, in the provided CAR, comprises a V_{H} region amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 5, and a V_{L} region comprising an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 6.

In some embodiments, the V_{H} region of the antibody or antigen-binding fragment thereof comprises a CDR-H1, a CDR-H2, a CDR-H3, respectively, comprising the amino acid sequences of CDR-H1, CDR-H2, and CDR-H3 contained within the V_{H} region amino acid sequence set forth in SEQ ID NO: 5; and comprises a CDR-L1, a CDR-L2, a CDR-L3, respectively, comprising the amino acid sequences of CDR-L1, CDR-L2, and CDR-L3, respectively contained within the V_{L} region amino acid sequence set forth in SEQ ID NO: 6.

In some embodiments, the V_{H} region of the antibody or antigen-binding fragment thereof comprise the amino acid sequence set forth in SEQ ID NO: 5, and the V_{L} region of the antibody or antigen-binding fragment comprises the amino acid sequence set forth in SEQ ID NO: 6. In some embodiments, the V_{H} and V_{L} regions of the antibody or antigen-binding fragment thereof comprise the amino acid sequences set forth in SEQ ID NO: 5 and 6, respectively, or any antibody or antigen-binding fragment thereof that has at least 90% sequence identity to any of the above V_{H} and V_{L}, such as at least 90%, 91%, 92%, 93%, 94%, 95%, 96%, 97%, 98%, or 99% sequence identity thereto.

For example, the V_{H} and V_{L} regions of the antibody or antigen-binding fragment thereof provided therein comprise the amino acid sequence set forth in SEQ ID NO: 5 and 6, respectively.

Among the CARs provided herein is a CAR in which the antibody, such as an anti-BAFF-R antibody, or antibody fragment, in the provided CAR, comprises a V_{H} region amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 7, and a V_{L} region comprising an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 8.

In some embodiments, the V_{H} region of the antibody or antigen-binding fragment thereof comprises a CDR-H1, a CDR-H2, a CDR-H3, respectively, comprising the amino acid sequences of CDR-H1, CDR-H2, and CDR-H3 contained within the V_{H} region amino acid sequence set forth in SEQ ID NO: 7; and comprises a CDR-L1, a CDR-L2, a CDR-L3, respectively, comprising the amino acid sequences of CDR-L1, CDR-L2, and CDR-L3, respectively contained within the V_{L} region amino acid sequence set forth in SEQ ID NO: 8.

In some embodiments, the V_{H} region of the antibody or antigen-binding fragment thereof comprise the amino acid sequence set forth in SEQ ID NO: 7, and the V_{L} region of the antibody or antigen-binding fragment comprises the amino acid sequence set forth in SEQ ID NO: 8. In some embodiments, the V_{H} and V_{L} regions of the antibody or antigen-binding fragment thereof comprise the amino acid sequences set forth in SEQ ID NO: 7 and 8, respectively, or any antibody or antigen-binding fragment thereof that has at least 90% sequence identity to any of the above V_{H} and V_{L}, such as at least 90%, 91%, 92%, 93%, 94%, 95%, 96%, 97%, 98%, or 99% sequence identity thereto.

For example, the V_{H} and V_{L} regions of the antibody or antigen-binding fragment thereof provided therein comprise the amino acid sequence set forth in SEQ ID NO: 7 and 8, respectively.

Among the CARs provided herein is a CAR in which the antibody, such as an anti-BAFF-R antibody, or antibody fragment, in the provided CAR, comprises a V_{H} region amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 9, and a V_{L} region comprising an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 10.

In some embodiments, the V_{H} region of the antibody or antigen-binding fragment thereof comprises a CDR-H1, a CDR-H2, a CDR-H3, respectively, comprising the amino acid sequences of CDR-H1, CDR-H2, and CDR-H3 contained within the V_{H} region amino acid sequence set forth in SEQ ID NO: 9; and comprises a CDR-L1, a CDR-L2, a CDR-L3, respectively, comprising the amino acid sequences of CDR-L1, CDR-L2, and CDR-L3, respectively contained within the V_{L} region amino acid sequence set forth in SEQ ID NO: 10.

In some embodiments, the V_{H} region of the antibody or antigen-binding fragment thereof comprise the amino acid sequence set forth in SEQ ID NO: 9, and the V_{L} region of the antibody or antigen-binding fragment comprises the amino acid sequence set forth in SEQ ID NO: 10. In some embodiments, the V_{H} and V_{L} regions of the antibody or antigen-binding fragment thereof comprise the amino acid sequences set forth in SEQ ID NO: 9 and 10, respectively, or any antibody or antigen-binding fragment thereof that has at least 90% sequence identity to any of the above V_{H} and V_{L}, such as at least 90%, 91%, 92%, 93%, 94%, 95%, 96%, 97%, 98%, or 99% sequence identity thereto.

For example, the V_{H} and V_{L} regions of the antibody or antigen-binding fragment thereof provided therein comprise the amino acid sequence set forth in SEQ ID NO: 9 and 10, respectively.

In some embodiments, the antibody or antigen-binding fragment thereof, in the provided CAR, is based on a single-chain antibody fragment, such as a single chain variable fragment (scFv) or a diabody or a single domain antibody (sdAb). In some embodiments, the antibody or antigen-binding fragment is a single domain antibody comprising only the V_{H} region. In some embodiments, the antibody or antigen binding fragment is an scFv comprising a heavy chain variable (V_{H}) region and a light chain variable (V_{L}) region. In some embodiments, a single-chain antibody fragment (e.g., scFv) includes one or more linkers joining two antibody domains or regions, such as a heavy chain variable (V_{H}) region and a light chain variable (V_{L}) region. The linker typically is a peptide linker, e.g., a flexible and/or soluble peptide linker. Among the linkers are those rich in glycine and serine and/or in some cases threonine. In some embodiments, the linkers further include charged residues such as lysine and/or glutamate, which can improve solubility. In some embodiments, the linkers further include one or more proline.

Accordingly, the provided CARs contain anti-BAFF-R antibodies that include single-chain antibody fragments, based on scFvs and diabodies, particularly human single-chain antibody fragments, typically comprising linker(s) joining two antibody domains or regions, such V_{H} and V_{L} regions. The provided CARs contain an anti-CD19 antibody that include single-chain antibody fragment, based on scFvs and diabodies, particularly human single-chain antibody fragments, typically comprising linker(s) joining two antibody domains or regions, such V_{H} and V_{L} regions.

The linker typically is a peptide linker, e.g., a flexible and/or soluble peptide linker, such as one rich in glycine and serine. In some embodiments, the BAFF-R-binding domain comprises a linker between the V_{H} and V_{L} regions. In some embodiments, in order from N- to C-terminus, the BAFF-R-binding domain comprises one of the V_{H} and V_{L} regions, a linker, and the other of the V_{H} and V_{L} regions. In some embodiments, the linker is set forth in SEQ ID NO: 58. Thus, in some embodiments, in order from N- to C-terminus, the BAFF-R-binding domain comprises one of the V_{H} and V_{L} regions, the linker set forth in SEQ ID NO: 58, and the other of the V_{H} and V_{L} regions.

In some aspects, the linkers rich in glycine and serine (and/or threonine) include at least 80%, 85%, 90%, 91%, 92%, 93%, 94%, 95%, 96%, 97%, 98%, or 99% such amino acid(s). In some embodiments, they include at least at or about 50%, 55%, 60%, 70%, or 75%, glycine, serine, and/or threonine. In some embodiments, the linker is comprised substantially entirely of glycine, serine, and/or threonine. The linkers generally are between about 5 and about 50 amino acids in length, typically between at or about 10 and at or about 30, e.g., 10, 11, 12, 13, 14, 15, 16, 17, 18, 19, 20, 21, 22, 23, 24, 25, 26, 27, 28, 29, or 30, and in some examples between 10 and 25 amino acids in length. Exemplary linkers include linkers having various numbers of repeats of the sequence GGGGS (4GS; SEQ ID NO: 60), such as between 2, 3, 4 and 5 repeats of such a sequence. Exemplary linkers include those having or consisting of an sequence set forth in SEQ ID NO: 58 (GGGGSGGGGSGGGGS). Exemplary linkers further include those having or consisting of the sequence set forth in SEQ ID NO: 59 (GSTSGSGKPGSGEGSTKG), SEQ ID NO: 61 (GGGGSGGGGS), and SEQ ID NO: 62 (GGGGSGGGGSGGGGSGGGGS).

Accordingly, in some embodiments, the provided embodiments include single-chain antibody fragments, e.g., V_{H} and V_{L} of an scFv, comprising one or more of the aforementioned linkers, such as glycine/serine rich linkers, including linkers having repeats of GGGS (SEQ ID NO: 60), such as the linker set forth in SEQ ID NO: 58, 61, or 62.

In some embodiments, the CAR comprises a loop format. In some embodiments, the V_{H} or V_{L} region of the CD19-binding domain is joined to the V_{H} or the V_{L} region of the BAFF-R-binding domain by a linker. In some embodiments, one of the V_{H} and the V_{L} region of the CD19-binding domain is joined to the other of the V_{H} and the V_{L} region of the CD19-binding domain by a linker. In some embodiments, one of the V_{H} and the V_{L} region of the BAFF-R-binding domain is joined to the other of the V_{H} and the V_{L} region of the BAFF-R-binding domain by a linker. In some embodiments, the linker is set forth in SEQ ID NO: 58. In some embodiments, the linker is set forth in SEQ ID NO: 59. In some embodiments, the linker is set forth in SEQ ID NO: 60. In some embodiments, the linker is set forth in SEQ ID NO: 61. In some embodiments, the linker is set forth in SEQ ID NO: 62.

In some embodiments, the V_{H} region of the CD19-binding domain is joined to the V_{L} region of the BAFF-R-binding domain by the linker set forth in SEQ ID NO: 62. In some embodiments, the V_{H} region of the CD19-binding domain is joined to the V_{L} region of the BAFF-R-binding domain by the linker set forth in SEQ ID NO: 60. In some embodiments, the V_{H} region of the CD19-binding domain is joined to the V_{L} region of the BAFF-R-binding domain by the linker set forth in SEQ ID NO: 61. In some embodiments, the V_{H} region of the CD19-binding domain is joined to the V_{H} region of the BAFF-R-binding domain by the linker set forth in SEQ ID NO: 62. In some embodiments, the V_{H} region of the CD19-binding domain is joined to the V_{H} region of the BAFF-R-binding domain by the linker set forth in SEQ ID NO: 60. In some embodiments, the V_{H} region of the CD19-binding domain is joined to the V_{H} region of the BAFF-R-binding domain by the linker set forth in SEQ ID NO: 61.

In some embodiments, the V_{L} region of the CD19-binding domain is joined to the V_{L} region of the BAFF-R-binding domain by the linker set forth in SEQ ID NO: 62. In some embodiments, the V_{L} region of the CD19-binding domain is joined to the V_{L} region of the BAFF-R-binding domain by the linker set forth in SEQ ID NO: 60. In some embodiments, the V_{L} region of the CD19-binding domain is joined to the V_{L} region of the BAFF-R-binding domain by the linker set forth in SEQ ID NO: 61. In some embodiments, the V_{L} region of the CD19-binding domain is joined to the V_{H} region of the BAFF-R-binding domain by the linker set forth in SEQ ID NO: 62. In some embodiments, the V_{L} region of the CD19-binding domain is joined to the V_{H} region of the BAFF-R-binding domain by the linker set forth in SEQ ID NO: 60. In some embodiments, the V_{L} region of the CD19-binding domain is joined to the V_{H} region of the BAFF-R-binding domain by the linker set forth in SEQ ID NO: 61.

Accordingly, in some embodiments, the provided embodiments include single-chain antibody fragments, e.g., V_{H} and V_{L} of an scFvs, comprising one or more of the aforementioned linkers, such as glycine/serine rich linkers, including linkers having repeats of GGGGS (SEQ ID NO: 60), such as the linker set forth in SEQ ID NO: 58, 61, or 62. In some embodiments, the linker comprises the sequence set forth in SEQ ID NO: 58. In some embodiments, the linker comprises the sequence set forth in SEQ ID NO: 60. In some embodiments, the linker comprises the sequence set forth in SEQ ID NO: 61. In some embodiments, the linker comprises the sequence set forth in SEQ ID NO: 62.

In some embodiments, the V_{H} region may be amino terminal to the V_{L} region. In some embodiments, the V_{H} region may be carboxy terminal to the V_{L} region. In particular embodiments, the fragment may include a V_{H} region or portion thereof, followed by the linker, followed by a V_{L} region or portion thereof. In other embodiments, the fragment may include the V_{L} region or portion thereof, followed by the linker, followed by the V_{H} region or portion thereof.

In some embodiments, the CAR comprises a linear format. Thus, in some embodiments, the CAR comprises a V_{H} and V_{L} of an anti-BAFF-R scFv and a V_{H} and V_{L} of an anti-CD19 scFv. In some embodiments, the V_{H} and V_{L} of the anti-BAFF-R scFv and the V_{H} and V_{L} of the anti-CD19 scFv are joined by a linker. In some embodiments, the linker is set forth in SEQ ID NO: 58. In some embodiments, the linker is set forth in SEQ ID NO: 60. In some embodiments, the linker is set forth in SEQ ID NO: 61. In some embodiments, the linker is set forth in SEQ ID NO:62. In some embodiments, the V_{H} and V_{L} regions of the anti-BAFF-R scFv are joined by the linker set forth in SEQ ID NO: 58. In some embodiments, the V_{H} and V_{L} regions of the anti-CD19 scFv are joined by the linker set forth in SEQ ID NO: 59.

In some aspects, a BAFF-R scFv provided herein comprises the amino acid sequence set forth in SEQ ID NOs: 11-15, or has an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NOs: 11-15. In some aspects, a BAFF-R scFv provided herein comprises the amino acid sequence set forth in SEQ ID NO: 11, or has an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 11. In some aspects, an scFv provided herein comprises the amino acid sequence set forth in SEQ ID NO: 11. In some aspects, an scFv provided herein comprises an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 11. In some aspects, an scFv provided herein comprises the amino acid sequence set forth in SEQ ID NO: 12, or has an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 12. In some aspects, an scFv provided herein comprises the amino acid sequence set forth in SEQ ID NO: 12. In some aspects, an scFv provided herein comprises an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 12.

In some aspects, a BAFF-R scFv provided herein comprises the amino acid sequence set forth in SEQ ID NO: 13, or has an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 13. In some aspects, an scFv provided herein comprises the amino acid sequence set forth in SEQ ID NO: 13. In some aspects, an scFv provided herein comprises an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 13. In some aspects, an scFv provided herein comprises the amino acid sequence set forth in SEQ ID NO: 14, or has an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 14. In some aspects, an scFv provided herein comprises the amino acid sequence set forth in SEQ ID NO: 14. In some aspects, an scFv provided herein comprises an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 14. In some aspects, a BAFF-R scFv provided herein comprises the amino acid sequence set forth in SEQ ID NO: 15, or has an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 15. In some aspects, an scFv provided herein comprises the amino acid sequence set forth in SEQ ID NO: 15. In some aspects, an scFv provided herein comprises an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 15.

Among the provided CARs is a CAR in which the BAFF-R-binding domain contains a V_{H} region comprising the sequence set forth in SEQ ID NO: 1 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO: 1; and contains a V_{L} region comprising the sequence set forth in SEQ ID NO: 2 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO: 2. In some embodiments, the BAFF-R-binding domain of the provided CAR contains a V_{H} region that has a CDRH1, a CDRH2 and a CDRH3 comprising the amino acid sequence of SEQ ID NOS: 16, 17, and 18, respectively and a V_{L} region that has a CDRL1, a CDRL2 and a CDRL3 comprising the amino acid sequence of SEQ ID NOS: 19, 20, and 21, respectively. In some embodiments, the V_{H} region comprises the sequence set forth in SEQ ID NO: 1 and the V_{L} region comprises the sequence set forth in SEQ ID NO: 2.

Among the provided CARs is a CAR in which the BAFF-R-binding domain contains a V_{H} region comprising the sequence set forth in SEQ ID NO: 3 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO: 3; and contains a V_{L} region comprising the sequence set forth in SEQ ID NO: 4 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO: 4. In some embodiments, the BAFF-R-binding domain of the provided CAR contains a V_{H} region that has a CDRH1, a CDRH2 and a CDRH3 comprising the amino acid sequence of SEQ ID NOS: 22, 23, and 24, respectively and a V_{L} region that has a CDRL1, a CDRL2 and a CDRL3 comprising the amino acid sequence of SEQ ID NOS: 25, 26, and 27, respectively. In some embodiments, the V_{H} region comprises the sequence set forth in SEQ ID NO: 3 and the V_{L} region comprises the sequence set forth in SEQ ID NO: 4.

Among the provided CARs is a CAR in which the BAFF-R-binding domain contains a V_{H} region comprising the sequence set forth in SEQ ID NO: 5 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO: 5; and contains a V_{L} region comprising the sequence set forth in SEQ ID NO: 6 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO: 6. In some embodiments, the BAFF-R-binding domain of the provided CAR contains a V_{H} region that has a CDRH1, a CDRH2 and a CDRH3 comprising the amino acid sequence of SEQ ID NOS: 28, 29, and 30, respectively and a V_{L} region that has a CDRL1, a CDRL2 and a CDRL3 comprising the amino acid sequence of SEQ ID NOS: 31, 26, and 27, respectively. In some embodiments, the V_{H} region comprises the sequence set forth in SEQ ID NO: 5 and the V_{L} region comprises the sequence set forth in SEQ ID NO: 6.

Among the provided CARs is a CAR in which the BAFF-R-binding domain contains a V_{H} region comprising the sequence set forth in SEQ ID NO: 7 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO: 7; and contains a V_{L} region comprising the sequence set forth in SEQ ID NO: 8 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO: 8. In some embodiments, the BAFF-R-binding domain of the provided CAR contains a V_{H} region that has a CDRH1, a CDRH2 and a CDRH3 comprising the amino acid sequence of SEQ ID NOS: 22, 32, and 24, respectively and a V_{L} region that has a CDRL1, a CDRL2 and a CDRL3 comprising the amino acid sequence of SEQ ID NOS: 33, 26, and 34, respectively. In some embodiments, the V_{H} region comprises the sequence set forth in SEQ ID NO: 7 and the V_{L} region comprises the sequence set forth in SEQ ID NO: 8.

Among the provided CARs is a CAR in which the BAFF-R-binding domain contains a V_{H} region comprising the sequence set forth in SEQ ID NO: 9 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO: 9; and contains a V_{L} region comprising the sequence set forth in SEQ ID NO: 10 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO: 10. In some embodiments, the BAFF-R-binding domain of the provided CAR contains a V_{H} region that has a CDRH1, a CDRH2 and a CDRH3 comprising the amino acid sequence of SEQ ID NOS: 35, 36, and 37, respectively and a V_{L} region that has a CDRL1, a CDRL2 and a CDRL3 comprising the amino acid sequence of SEQ ID NOS: 38, 39, and 40, respectively. In some embodiments, the V_{H} region comprises the sequence set forth in SEQ ID NO: 9 and the V_{L} region comprises the sequence set forth in SEQ ID NO: 10.

Among the antibodies, e.g., antigen-binding fragments, in the provided CARs, are human antibodies. In some embodiments of a provided human anti-BAFF-R antibody, e.g., antigen-binding fragments, the human antibody contains a V_{H} region that comprises a portion having at least 95%, 96%, 97%, 98%, 99%, or 100% sequence identity to an amino acid sequence encoded by a germline nucleotide human heavy chain V segment, a portion having at least 95%, 96%, 97%, 98%, 99%, or 100% sequence identity to an amino acid sequence encoded by a germline nucleotide human heavy chain D segment, and/or a portion having at least 95%, 96%, 97%, 98%, 99%, or 100% sequence identity to an amino acid sequence encoded by a germline nucleotide human heavy chain J segment; and/or contains a V_{L} region that comprises a portion having at least 95%, 96%, 97%, 98%, 99%, or 100% sequence identity to an amino acid sequence encoded by a germline nucleotide human kappa or lambda chain V segment, and/or a portion having at least 95%, 96%, 97%, 98%, 99%, or 100% sequence identity to an amino acid sequence encoded by a germline nucleotide human kappa or lambda chain J segment. In some embodiments, the portion of the V_{H} region corresponds to the CDR-H1, CDR-H2 and/or CDR-H3. In some embodiments, the portion of the V_{H} region corresponds to the framework region 1 (FR1), FR2, FR2 and/or FR4. In some embodiments, the portion of the V_{L} region corresponds to the CDR-L1, CDR-L2 and/or CDR-L3. In some embodiments, the portion of the V_{L} region corresponds to the FR1, FR2, FR2 and/or FR4.

In some embodiments, the human antibody, e.g., antigen-binding fragment, contains a CDR-H1 having at least 95%, 96%, 97%, 98%, 99%, or 100% sequence identity to an amino acid sequence of the corresponding CDR-H1 region within a sequence encoded by a germline nucleotide human heavy chain V segment. For example, the human antibody in some embodiments contains a CDR-H1 having a sequence that is 100% identical or with no more than one, two or three amino acid differences as compared to the corresponding CDR-H1 region within a sequence encoded by a germline nucleotide human heavy chain V segment.

In some embodiments, the human antibody, e.g., antigen-binding fragment, contains a CDR-H2 having at least 95%, 96%, 97%, 98%, 99%, or 100% sequence identity to an amino acid sequence of the corresponding CDR-H2 region within a sequence encoded by a germline nucleotide human heavy chain V segment. For example, the human antibody in some embodiments contains a CDR-H2 having a sequence that is 100% identical or with no more than one, two or three amino acid difference as compared to the corresponding CDR-H2 region within a sequence encoded by a germline nucleotide human heavy chain V segment.

In some embodiments, the human antibody, e.g., antigen-binding fragment, contains a CDR-H3 having at least 95%, 96%, 97%, 98%, 99%, or 100% sequence identity to an amino acid sequence of the corresponding CDR-H3 region within a sequence encoded by a germline nucleotide human heavy chain V segment, D segment and J segment. For example, the human antibody in some embodiments contains a CDR-H3 having a sequence that is 100% identical or with no more than one, two or three amino acid differences as compared to the corresponding CDR-H3 region within a sequence encoded by a germline nucleotide human heavy chain V segment, D segment and J segment.

In some embodiments, the human antibody, e.g., antigen-binding fragment, contains a CDR-L1 having at least 95%, 96%, 97%, 98%, 99%, or 100% sequence identity to an amino acid sequence of the corresponding CDR-LI region within a sequence encoded by a germline nucleotide human light chain V segment. For example, the human antibody in some embodiments contains a CDR-L1 having a sequence that is 100% identical or with no more than one, two or three amino acid differences as compared to the corresponding CDR-L1 region within a sequence encoded by a germline nucleotide human light chain V segment.

In some embodiments, the human antibody, e.g., antigen-binding fragment, contains a CDR-L2 having at least 95%, 96%, 97%, 98%, 99%, or 100% sequence identity to an amino acid sequence of the corresponding CDR-L2 region within a sequence encoded by a germline nucleotide human light chain V segment. For example, the human antibody in some embodiments contains a CDR-L2 having a sequence that is 100% identical or with no more than one, two or three amino acid difference as compared to the corresponding CDR-L2 region within a sequence encoded by a germline nucleotide human light chain V segment.

In some embodiments, the human antibody, e.g., antigen-binding fragment, contains a CDR-L3 having at least 95%, 96%, 97%, 98%, 99%, or 100% sequence identity to an amino acid sequence of the corresponding CDR-L3 region within a sequence encoded by a germline nucleotide human light chain V segment and J segment. For example, the human antibody in some embodiments contains a CDR-L3 having a sequence that is 100% identical or with no more than one, two or three amino acid differences as compared to the corresponding CDR-L3 region within a sequence encoded by a germline nucleotide human light chain V segment and J segment.

In some embodiments, the human antibody, e.g., antigen-binding fragment, contains a framework region that contains human germline gene segment sequences. For example, in some embodiments, the human antibody contains a V_{H} region in which the framework region, e.g. FR1, FR2, FR3 and FR4, has at least 95%, 96%, 97%, 98%, 99%, or 100% sequence identity to a framework region encoded by a human germline antibody segment, such as a V segment and/or J segment. In some embodiments, the human antibody contains a V_{L} region in which the framework region e.g. FR1, FR2, FR3 and FR4, has at least 95%, 96%, 97%, 98%, 99%, or 100% sequence identity to a framework region encoded by a human germline antibody segment, such as a V segment and/or J segment. For example, in some such embodiments, the framework region sequence contained within the V_{H} region and/or V_{L} region differs by no more than 10 amino acids, such as no more than 9, 8, 7, 6, 5, 4, 3, 2 or 1 amino acid, compared to the framework region sequence encoded by a human germline antibody segment.

b. CD19-Binding Domain

In some embodiments, the CAR includes a CD19-binding domain comprising an antibody, such as a heavy chain variable (V_{H}) region and/or light chain variable (V_{L}) region of the antibody. In some embodiments, the (V_{H}) region and the (V_{L}) region of the CD19-binding domain are joined by an intradomain linker. In some embodiments, the (V_{H}) region and the (V_{L}) region of the CD19-binding domain comprise an scFv antibody fragment. In some embodiments, the provided CD19-binding CARs contain an antibody, such as an anti-CD19 antibody, or an antigen-binding fragment thereof that confers the CD19-binding properties of the provided CAR.

In some embodiments, the antibody, e.g., the anti-CD19 antibody, or antigen-binding fragment, contains a heavy and/or light chain variable (V_{H} or V_{L}) region sequence as described, or a sufficient antigen-binding portion thereof. In some embodiments, the anti-CD19 antibody, e.g., antigen-binding fragment, contains a V_{H} region sequence or sufficient antigen-binding portion thereof that contains a CDR-H1, CDR-H2 and/or CDR-H3 as described. In some embodiments, the anti-CD19 antibody, e.g., antigen-binding fragment, contains a V_{L} region sequence or sufficient antigen-binding portion that contains a CDR-L1, CDR-L2 and/or CDR-L3 as described. In some embodiments, the anti-CD19 antibody, e.g., antigen-binding fragment, contains a V_{H} region sequence that contains a CDR-H1, CDR-H2 and/or CDR-H3 as described and contains a V_{L} region sequence that contains a CDR-L1, CDR-L2 and/or CDR-L3 as described. Also among the antibodies are those having sequences at least at or about 90%, at or about 91%, at or about 92%, at or about 93%, at or about 94%, at or about 95%, at or about 96%, at or about 97%, at or about 98%, or at or about 99% identical to such a sequence.

In some embodiments, the V_{H} and V_{L} region of the anti-CD19 antibody in a provided CAR is the V_{H} and V_{L} sequence of a CAR T cell therapy that targets CD19. Exemplary CAR T cell therapies that target CD19 include those investigated or being investigated in clinical trials NCT02644655, NCT03744676, NCT01087294, NCT03366350, NCT03790891, NCT03497533, NCT04007029, NCT03960840, NCT04049383, NCT04094766, NCT03366324, NCT02546739, NCT03448393, NCT03467256, NCT03488160, NCT04012879, NCT03016377, NCT03468153, NCT03483688, NCT03398967, NCT03229876, NCT03455972, NCT03423706, NCT03497533, and NCT04002401, including FDA-approved products BREYANZI® (lisocabtagene maraleucel), TECARTUS™ (brexucabtagene autoleucel), KYMRIAH™ (tisagenlecleucel), and YESCARTA™ (axicabtagene ciloleucel).

In some embodiments, the V_{H} and a V_{L} is derived from an antibody or an antibody fragment specific to CD19. In some embodiments, the antibody or antibody fragment that binds CD19 is a mouse derived antibody such as FMC63 and SJ25C1. In some embodiments, exemplary antibody or antibody fragment include human anti-CD19 antibodies, such as those described in U.S. Patent Publication No. WO 2014/031687, US 2016/0152723 and WO 2016/033570, the contents of each of which are incorporated by reference in their entirety.

In some embodiments the antigen-binding domain includes a V_{H} and/or V_{L} derived from FMC63, which, in some aspects, can be an scFv. In some embodiments the scFv and/or V_{H} domains is derived from FMC63. FMC63 generally refers to a mouse monoclonal IgG1 antibody raised against Nalm-1 and -16 cells expressing CD19 of human origin (Ling, N. R., et al. (1987). Leucocyte typing III. 302). The FMC63 antibody comprises CDRH1 and H2 set forth in SEQ ID NOS: 43 and 44 respectively, and CDRH3 set forth in SEQ ID NOS: 45 or 46, and CDRL1 set forth in SEQ ID NOS: 47 and CDR L2 set forth in SEQ ID NO: 48, or 49, and CDR L3 sequences set forth in SEQ ID NO: 50, or 51. The FMC63 antibody comprises the heavy chain variable region (V_{H}) comprising the amino acid sequence of SEQ ID NO: 41 and the light chain variable region (V_{L}) comprising the amino acid sequence of SEQ ID NO: 42. In some embodiments, the scFv comprises a variable light chain containing the CDRL1 sequence of SEQ ID NO:47, a CDRL2 sequence of SEQ ID NO: 48, and a CDRL3 sequence of SEQ ID NO:50 and/or a variable heavy chain containing a CDRH1 sequence of SEQ ID NO:43, a CDRH2 sequence of SEQ ID NO:44, and a CDRH3 sequence of SEQ ID NO:45. In some embodiments, the scFv comprises a variable heavy chain region of FMC63 set forth in SEQ ID NO:41 and a variable light chain region of FMC63 set forth in SEQ ID NO:42.

In some embodiments, the anti-CD19 binding domain of the CAR includes the V_{H} and V_{L} sequences of the antigen-binding domain of the anti-CD19 CAR of BREYANZI® (lisocabtagene maraleucel).

In some embodiments, the anti-CD19 binding domain of the CAR includes the V_{H} and V_{L} sequences of the antigen-binding domain of the anti-CD19 CAR of TECARTUS™ (brexucabtagene autoleucel).

In some embodiments, the anti-CD19 binding domain of the CAR includes the V_{H} and V_{L} sequences of the antigen-binding domain of the anti-CD19 CAR of KYMRIAH™ (tisagenlecleucel).

In some embodiments, the anti-CD19 binding domain of the CAR includes the V_{H} and V_{L} sequences of the antigen-binding domain of the anti-CD19 CAR of YESCARTA™ (axicabtagene ciloleucel).

In some embodiments the anti-CD19 antigen-binding domain of the provided CAR includes a V_{H} and/or V_{L} derived from SJ25C1, which, in some aspects, can be an scFv. SJ25C1 is a mouse monoclonal IgG1 antibody raised against Nalm-1 and -16 cells expressing CD19 of human origin (Ling, N. R., et al. (1987). Leucocyte typing III. 302). The SJ25C1 antibody comprises CDRH1, H2 and H3 set forth in SEQ ID NOS: 124-126, respectively, and CDRL1, L2 and L3 sequences set forth in SEQ ID NOS: 121-123, respectively. The SJ25C1 antibody comprises the heavy chain variable region (V_{H}) comprising the amino acid sequence of SEQ ID NO: 127 and the light chain variable region (V_{L}) comprising the amino acid sequence of SEQ ID NO: 128. In some embodiments, the scFv comprises a variable light chain containing the CDRL1 sequence of SEQ ID NO:121, a CDRL2 sequence of SEQ ID NO: 122, and a CDRL3 sequence of SEQ ID NO:123 and/or a variable heavy chain containing a CDRH1 sequence of SEQ ID NO:124, a CDRH2 sequence of SEQ ID NO:125, and a CDRH3 sequence of SEQ ID NO:126. In some embodiments, the scFv comprises a variable heavy chain region of SJ25C1 set forth in SEQ ID NO:127 and a variable light chain region of SJ25C1 set forth in SEQ ID NO:128.

In some embodiments, the CAR contains an antibody or antigen-binding fragment thereof, that has a heavy chain variable (V_{H}) region having the amino acid sequence set forth in SEQ ID NO: 41 or an amino acid sequence that has at least at or about 90%, at or about 91%, at or about 92%, at or about 93%, at or about 94%, at or about 95%, at or about 96%, at or about 97%, at or about 98%, or at or about 99% sequence identity to the V_{H} region amino acid set forth in SEQ ID NO: 41 or contains a CDR-H1, CDR-H2, and/or CDR-H3 present in such a V_{H} sequence.

In some embodiments, the CAR contains an antibody or antigen-binding fragment thereof, that has a heavy chain variable (V_{H}) region having the amino acid sequence set forth in SEQ ID NO: 63 or an amino acid sequence that has at least at or about 90%, at or about 91%, at or about 92%, at or about 93%, at or about 94%, at or about 95%, at or about 96%, at or about 97%, at or about 98%, or at or about 99% sequence identity to the V_{H} region amino acid set forth in SEQ ID NO: 63 or contains a CDR-H1, CDR-H2, and/or CDR-H3 present in such a V_{H} sequence.

In some embodiments, the CAR contains an antibody or antigen-binding fragment thereof, that has a heavy chain variable (V_{H}) region having the amino acid sequence set forth in SEQ ID NO: 64 or an amino acid sequence that has at least at or about 90%, at or about 91%, at or about 92%, at or about 93%, at or about 94%, at or about 95%, at or about 96%, at or about 97%, at or about 98%, or at or about 99% sequence identity to the V_{H} region amino acid set forth in SEQ ID NO: 64 or contains a CDR-H1, CDR-H2, and/or CDR-H3 present in such a V_{H} sequence.

In some embodiments, the V_{H} region of an antibody or antigen-binding fragment thereof comprises a CDR-H1, CDR-H2, and/or CDR-H3 according to Kabat numbering. In some embodiments, the V_{H} region of an antibody or antigen-binding fragment thereof comprises a CDR-H1, CDR-H2, and/or CDR-H3 according to Chothia numbering. In some embodiments, the V_{H} region of an antibody or antigen-binding fragment thereof comprises a CDR-H1, CDR-H2, and/or CDR-H3 according to AbM numbering.

In some embodiments, the CAR contains an antibody or antigen-binding fragment thereof, that has a variable heavy chain (V_{H}) region comprising a CDR-H1 comprising the amino acid sequence set forth in SEQ ID NO: 43, a CDR-H2 comprising the amino acid sequence set forth in SEQ ID NO: 44, and a CDR-H3 comprising the amino acid sequence set forth in SEQ ID NO: 45 or 46. In some embodiments, the antibody or antigen-binding fragment thereof comprises a V_{H} region comprising a CDR-H1, a CDR-H2, and a CDR-H3 comprising the amino acid sequence set forth in SEQ ID NO:43, 44, and 45, respectively or 43, 44, and 46 respectively. In some embodiments, the antibody or antigen-binding fragment thereof comprises a V_{H} region comprising the amino acid sequence set forth in SEQ ID NO: 43, 44, and 45. In some embodiments, the antibody or antigen-binding fragment thereof comprises a V_{H} region comprising the amino acid sequence set forth in SEQ ID NO: 43, 44, and 46. In some embodiments, the antibody or antigen-binding fragment thereof comprises a CDR-H1, CDR-H2 and CDR-H3, respectively, comprising the amino acid sequence of a CDR-H1, a CDR-H2, and a CDR-H3 contained within the V_{H} region amino acid sequence set forth in SEQ ID NO: 41.

In some embodiments of the antibody or antigen-binding fragment thereof provided herein, the V_{H} region comprises any of the CDR-H1, CDR-H2 and CDR-H3 as described and comprises a framework region 1 (FR1), a FR2, a FR3 and/or a FR4 having at least at or about 90%, at or about 91%, at or about 92%, at or about 93%, at or about 94%, at or about 95%, at or about 96%, at or about 97%, at or about 98%, or at or about 99% sequence identity, respectively, to a FR1, a FR2, a FR3 and/or a FR4 contained within the V_{H} region amino acid sequence set forth in SEQ ID NO: 41. In some embodiments, the antibody or antigen-binding fragment thereof comprises a V_{H} region comprising the amino acid sequence set forth in SEQ ID NO: 41.

In some embodiments, the CAR contains an antibody or antigen-binding fragment thereof, that has a variable heavy chain (V_{H}) region comprising a CDR-H1 comprising the amino acid sequence set forth in SEQ ID NO: 75, a CDR-H2 comprising the amino acid sequence set forth in SEQ ID NO: 76, and a CDR-H3 comprising the amino acid sequence set forth in SEQ ID NO: 77. In some embodiments, the antibody or antigen-binding fragment thereof comprises a V_{H} region comprising a CDR-H1, a CDR-H2, and a CDR-H3 comprising the amino acid sequence set forth in SEQ ID NO: 75, 76, and 77, respectively. In some embodiments, the antibody or antigen-binding fragment thereof comprises a V_{H} region comprising the amino acid sequence set forth in SEQ ID NO: 75, 76, and 77. In some embodiments, the antibody or antigen-binding fragment thereof comprises a CDR-H1, CDR-H2 and CDR-H3, respectively, comprising the amino acid sequence of a CDR-H1, a CDR-H2, and a CDR-H3 contained within the V_{H} region amino acid sequence set forth in SEQ ID NO: 63.

In some embodiments of the antibody or antigen-binding fragment thereof provided herein, the V_{H} region comprises any of the CDR-H1, CDR-H2 and CDR-H3 as described and comprises a framework region 1 (FR1), a FR2, a FR3 and/or a FR4 having at least at or about 90%, at or about 91%, at or about 92%, at or about 93%, at or about 94%, at or about 95%, at or about 96%, at or about 97%, at or about 98%, or at or about 99% sequence identity, respectively, to a FR1, a FR2, a FR3 and/or a FR4 contained within the V_{H} region amino acid sequence set forth in SEQ ID NO: 63. In some embodiments, the antibody or antigen-binding fragment thereof comprises a V_{H} region comprising the amino acid sequence set forth in SEQ ID NO: 63.

In some embodiments, the CAR contains an antibody or antigen-binding fragment thereof, that has a variable heavy chain (V_{H}) region comprising a CDR-H1 comprising the amino acid sequence set forth in SEQ ID NO: 75, a CDR-H2 comprising the amino acid sequence set forth in SEQ ID NO: 76, and a CDR-H3 comprising the amino acid sequence set forth in SEQ ID NO: 77. In some embodiments, the antibody or antigen-binding fragment thereof comprises a V_{H} region comprising a CDR-H1, a CDR-H2, and a CDR-H3 comprising the amino acid sequence set forth in SEQ ID NO: 75, 76, and 77, respectively. In some embodiments, the antibody or antigen-binding fragment thereof comprises a V_{H} region comprising the amino acid sequence set forth in SEQ ID NO: 75, 76, and 77. In some embodiments, the antibody or antigen-binding fragment thereof comprises a CDR-H1, CDR-H2 and CDR-H3, respectively, comprising the amino acid sequence of a CDR-H1, a CDR-H2, and a CDR-H3 contained within the V_{H} region amino acid sequence set forth in SEQ ID NO: 64.

In some embodiments of the antibody or antigen-binding fragment thereof provided herein, the V_{H} region comprises any of the CDR-H1, CDR-H2 and CDR-H3 as described and comprises a framework region 1 (FR1), a FR2, a FR3 and/or a FR4 having at least at or about 90%, at or about 91%, at or about 92%, at or about 93%, at or about 94%, at or about 95%, at or about 96%, at or about 97%, at or about 98%, or at or about 99% sequence identity, respectively, to a FR1, a FR2, a FR3 and/or a FR4 contained within the V_{H} region amino acid sequence set forth in SEQ ID NO: 64. In some embodiments, the antibody or antigen-binding fragment thereof comprises a V_{H} region comprising the amino acid sequence set forth in SEQ ID NO: 64.

In some embodiments, the antibody or antibody fragment, in the provided CAR comprising a V_{H} region further comprises a light chain or a sufficient antigen binding portion thereof. For example, in some embodiments, the antibody or antigen-binding fragment thereof contains a V_{H} region and a V_{L} region, or a sufficient antigen-binding portion of a V_{H} and V_{L} region. In such embodiments, a V_{H} region sequence can be any of the above described V_{H} sequence. In some such embodiments, the antibody is an antigen-binding fragment, such as a Fab or an scFv. In some such embodiments, the antibody is a full-length antibody that also contains a constant region.

In some embodiments, a CAR provided herein, contains an antibody such as an anti-CD19 antibody, or antigen-binding fragment thereof that contains any of the above V_{H} region and contains a variable light chain region or a sufficient antigen binding portion thereof. For example, in some embodiments, the CAR contains an antibody or antigen-binding fragment thereof that contains a V_{H} region and a variable light chain (V_{L}) region, or a sufficient antigen-binding portion of a V_{H} and V_{L} region. In such embodiments, a V_{H} region sequence can be any of the above described V_{H} sequence. In some such embodiments, the antibody is an antigen-binding fragment, such as a Fab or an scFv. In some such embodiments, the antibody is a full-length antibody that also contains a constant region.

In some embodiments, the CAR contains an antibody or antigen-binding fragment thereof, that has a light chain variable (V_{L}) region having the amino acid sequence set forth in SEQ ID NO: 42, or an amino acid sequence that has at least at or about 90%, at or about 91%, at or about 92%, at or about 93%, at or about 94%, at or about 95%, at or about 96%, at or about 97%, at or about 98%, or at or about 99% sequence identity to the V_{L} region amino acid set forth in SEQ ID NO: 42, or contains a CDR-L1, CDR-L2, and/or CDR-L3 present in such a V_{L} sequence.

In some embodiments, the CAR contains an antibody or antigen-binding fragment thereof, that has a light chain variable (V_{L}) region having the amino acid sequence set forth in SEQ ID NO: 65, or an amino acid sequence that has at least at or about 90%, at or about 91%, at or about 92%, at or about 93%, at or about 94%, at or about 95%, at or about 96%, at or about 97%, at or about 98%, or at or about 99% sequence identity to the V_{L} region amino acid set forth in SEQ ID NO: 65, or contains a CDR-L1, CDR-L2, and/or CDR-L3 present in such a V_{L} sequence.

In some embodiments, the CAR contains an antibody or antigen-binding fragment thereof, that has a light chain variable (V_{L}) region having the amino acid sequence set forth in SEQ ID NO: 66, or an amino acid sequence that has at least at or about 90%, at or about 91%, at or about 92%, at or about 93%, at or about 94%, at or about 95%, at or about 96%, at or about 97%, at or about 98%, or at or about 99% sequence identity to the V_{L} region amino acid set forth in SEQ ID NO: 66, or contains a CDR-L1, CDR-L2, and/or CDR-L3 present in such a V_{L} sequence.

In some embodiments, the CAR contains an antibody or antigen-binding fragment thereof, that has a light chain variable (V_{L}) region having the amino acid sequence set forth in SEQ ID NO: 67, or an amino acid sequence that has at least at or about 90%, at or about 91%, at or about 92%, at or about 93%, at or about 94%, at or about 95%, at or about 96%, at or about 97%, at or about 98%, or at or about 99% sequence identity to the V_{L} region amino acid set forth in SEQ ID NO: 67, or contains a CDR-L1, CDR-L2, and/or CDR-L3 present in such a V_{L} sequence.

In some embodiments, the CAR contains an antibody or antigen-binding fragment thereof, that has a light chain variable (V_{L}) region having the amino acid sequence set forth in SEQ ID NO: 68, or an amino acid sequence that has at least at or about 90%, at or about 91%, at or about 92%, at or about 93%, at or about 94%, at or about 95%, at or about 96%, at or about 97%, at or about 98%, or at or about 99% sequence identity to the V_{L} region amino acid set forth in SEQ ID NO: 68, or contains a CDR-L1, CDR-L2, and/or CDR-L3 present in such a V_{L} sequence.

In some embodiments, the CAR contains an antibody or antigen-binding fragment thereof, that has a light chain variable (V_{L}) region having the amino acid sequence set forth in SEQ ID NO: 69, or an amino acid sequence that has at least at or about 90%, at or about 91%, at or about 92%, at or about 93%, at or about 94%, at or about 95%, at or about 96%, at or about 97%, at or about 98%, or at or about 99% sequence identity to the V_{L} region amino acid set forth in SEQ ID NO: 69, or contains a CDR-L1, CDR-L2, and/or CDR-L3 present in such a V_{L} sequence.

In some embodiments, the V_{L} region of an antibody or antigen-binding fragment thereof comprises a CDR-L1, CDR-L2, and/or CDR-L3 according to Kabat numbering. In some embodiments, the V_{L} region of an antibody or antigen-binding fragment thereof comprises a CDR-L1, CDR-L2, and/or CDR-L3 according to Chothia numbering. In some embodiments, the V_{L} region of an antibody or antigen-binding fragment thereof comprises a CDR-L1, CDR-L2, and/or CDR-L3 according to AbM numbering.

In some embodiments, the CAR contains an antibody or antigen-binding fragment thereof, that has a variable light chain (V_{L}) region comprising a CDR-L1 comprising the amino acid sequence set forth in SEQ ID NO: 47, a CDR-L2 comprising the amino acid sequence set forth in SEQ ID NO:48 or 49, and a CDR-L3 comprising the amino acid sequence set forth in SEQ ID NO: 50 or 51. In some embodiments, the antibody or antigen-binding fragment thereof comprises a V_{L} region comprising a CDR-L1, a CDR-L2, and a CDR-L3 comprising the amino acid sequence set forth in SEQ ID NO: 47, 48, and 50, respectively; 47, 49, and 50, respectively; 47, 48, and 51, respectively; or 47, 49, and 51, respectively.

In some embodiments, the antibody or antigen-binding fragment thereof contains a CDR-L1, CDR-L2, and CDR-L3, respectively, contained within the V_{L} region amino acid sequence set forth in SEQ ID NO: 42.

In some embodiments, the CAR contains an antibody or antigen-binding fragment thereof, that has a variable light chain (V_{L}) region comprising a CDR-L1 comprising the amino acid sequence set forth in SEQ ID NO: 78, a CDR-L2 comprising the amino acid sequence set forth in SEQ ID NO:79, and a CDR-L3 comprising the amino acid sequence set forth in SEQ ID NO: 80. In some embodiments, the antibody or antigen-binding fragment thereof comprises a V_{L} region comprising a CDR-L1, a CDR-L2, and a CDR-L3 comprising the amino acid sequence set forth in SEQ ID NO: 78, 79, and 80, respectively. In some embodiments, the antibody or antigen-binding fragment thereof contains a CDR-L1, CDR-L2, and CDR-L3, respectively, contained within the V_{L} region amino acid sequence set forth in SEQ ID NO: 65.

In some embodiments, the CAR contains an antibody or antigen-binding fragment thereof, that has a variable light chain (V_{L}) region comprising a CDR-L1 comprising the amino acid sequence set forth in SEQ ID NO: 81, a CDR-L2 comprising the amino acid sequence set forth in SEQ ID NO: 82, and a CDR-L3 comprising the amino acid sequence set forth in SEQ ID NO: 83. In some embodiments, the antibody or antigen-binding fragment thereof comprises a V_{L} region comprising a CDR-L1, a CDR-L2, and a CDR-L3 comprising the amino acid sequence set forth in SEQ ID NO: 81, 82, and 83, respectively. In some embodiments, the antibody or antigen-binding fragment thereof contains a CDR-L1, CDR-L2, and CDR-L3, respectively, contained within the V_{L} region amino acid sequence set forth in SEQ ID NO: 66.

In some embodiments, the CAR contains an antibody or antigen-binding fragment thereof, that has a variable light chain (V_{L}) region comprising a CDR-L1 comprising the amino acid sequence set forth in SEQ ID NO: 84, a CDR-L2 comprising the amino acid sequence set forth in SEQ ID NO: 85, and a CDR-L3 comprising the amino acid sequence set forth in SEQ ID NO: 86. In some embodiments, the antibody or antigen-binding fragment thereof comprises a V_{L} region comprising a CDR-L1, a CDR-L2, and a CDR-L3 comprising the amino acid sequence set forth in SEQ ID NO: 84, 85, and 86, respectively. In some embodiments, the antibody or antigen-binding fragment thereof contains a CDR-L1, CDR-L2, and CDR-L3, respectively, contained within the V_{L} region amino acid sequence set forth in SEQ ID NO: 67.

In some embodiments, the CAR contains an antibody or antigen-binding fragment thereof, that has a variable light chain (V_{L}) region comprising a CDR-L1 comprising the amino acid sequence set forth in SEQ ID NO: 84, a CDR-L2 comprising the amino acid sequence set forth in SEQ ID NO:85, and a CDR-L3 comprising the amino acid sequence set forth in SEQ ID NO: 87. In some embodiments, the antibody or antigen-binding fragment thereof comprises a V_{L} region comprising a CDR-L1, a CDR-L2, and a CDR-L3 comprising the amino acid sequence set forth in SEQ ID NO: 84, 85, and 87, respectively. In some embodiments, the antibody or antigen-binding fragment thereof contains a CDR-L1, CDR-L2, and CDR-L3, respectively, contained within the V_{L} region amino acid sequence set forth in SEQ ID NO: 68.

In some embodiments, the CAR contains an antibody or antigen-binding fragment thereof, that has a variable light chain (V_{L}) region comprising a CDR-L1 comprising the amino acid sequence set forth in SEQ ID NO: 88, a CDR-L2 comprising the amino acid sequence set forth in SEQ ID NO:89, and a CDR-L3 comprising the amino acid sequence set forth in SEQ ID NO: 90. In some embodiments, the antibody or antigen-binding fragment thereof comprises a V_{L} region comprising a CDR-L1, a CDR-L2, and a CDR-L3 comprising the amino acid sequence set forth in SEQ ID NO: 88, 89, and 90, respectively. In some embodiments, the antibody or antigen-binding fragment thereof contains a CDR-L1, CDR-L2, and CDR-L3, respectively, contained within the V_{L} region amino acid sequence set forth in SEQ ID NO: 69.

Among the CARs provided herein is a CAR in which the antibody, such as an anti-CD19 antibody, or antibody fragment, in the provided CAR, comprises a V_{H} region amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NOs: 41 and a V_{L} region comprising an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 42.

In some embodiments, the V_{H} region of the antibody or antigen-binding fragment thereof comprises a CDR-H1, a CDR-H2, a CDR-H3, respectively, comprising the amino acid sequences of CDR-H1, CDR-H2, and CDR-H3 contained within the V_{H} region amino acid sequence set forth in SEQ ID NO: 41; and comprises a CDR-L1, a CDR-L2, a CDR-L3, respectively, comprising the amino acid sequences of CDR-L1, CDR-L2, and CDR-L3, respectively contained within the V_{L} region amino acid sequence set forth in SEQ ID NO: 42.

In some embodiments, the V_{H} region of the antibody or antigen-binding fragment thereof comprise the amino acid sequence set forth in SEQ ID NO: 41 and the V_{L} region of the antibody or antigen-binding fragment comprises the amino acid sequence set forth in SEQ ID NO: 42. In some embodiments, the V_{H} and V_{L} regions of the antibody or antigen-binding fragment thereof comprise the amino acid sequences set forth in SEQ ID NO: 41 and 42, respectively, or any antibody or antigen-binding fragment thereof that has at least 90% sequence identity to any of the above V_{H} and V_{L}, such as at least 90%, 91%, 92%, 93%, 94%, 95%, 96%, 97%, 98%, or 99% sequence identity thereto.

For example, the V_{H} and V_{L} regions of the antibody or antigen-binding fragment thereof provided therein comprise the amino acid sequence set forth in SEQ ID NO: 41 and 42, respectively.

Among the CARs provided herein is a CAR in which the antibody, such as an anti-CD19 antibody, or antibody fragment, in the provided CAR, comprises a V_{H} region amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NOs: 63, and a V_{L} region comprising an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 65.

In some embodiments, the V_{H} region of the antibody or antigen-binding fragment thereof comprises a CDR-H1, a CDR-H2, a CDR-H3, respectively, comprising the amino acid sequences of CDR-H1, CDR-H2, and CDR-H3 contained within the V_{H} region amino acid sequence set forth in SEQ ID NO: 63; and comprises a CDR-L1, a CDR-L2, a CDR-L3, respectively, comprising the amino acid sequences of CDR-L1, CDR-L2, and CDR-L3, respectively contained within the V_{L} region amino acid sequence set forth in SEQ ID NO: 65.

In some embodiments, the V_{H} region of the antibody or antigen-binding fragment thereof comprise the amino acid sequence set forth in SEQ ID NOs: 63 and the V_{L} region of the antibody or antigen-binding fragment comprises the amino acid sequence set forth in SEQ ID NO: 65. In some embodiments, the V_{H} and V_{L} regions of the antibody or antigen-binding fragment thereof comprise the amino acid sequences set forth in SEQ ID NO: 63 and 65, respectively, or any antibody or antigen-binding fragment thereof that has at least 90% sequence identity to any of the above V_{H} and V_{L}, such as at least 90%, 91%, 92%, 93%, 94%, 95%, 96%, 97%, 98%, or 99% sequence identity thereto.

For example, the V_{H} and V_{L} regions of the antibody or antigen-binding fragment thereof provided therein comprise the amino acid sequence set forth in SEQ ID NO: 63 and 65, respectively.

Among the CARs provided herein is a CAR in which the antibody, such as an anti-CD19 antibody, or antibody fragment, in the provided CAR, comprises a V_{H} region amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NOs: 63, and a V_{L} region comprising an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 66.

In some embodiments, the V_{H} region of the antibody or antigen-binding fragment thereof comprises a CDR-H1, a CDR-H2, a CDR-H3, respectively, comprising the amino acid sequences of CDR-H1, CDR-H2, and CDR-H3 contained within the V_{H} region amino acid sequence set forth in SEQ ID NO: 63; and comprises a CDR-L1, a CDR-L2, a CDR-L3, respectively, comprising the amino acid sequences of CDR-L1, CDR-L2, and CDR-L3, respectively contained within the V_{L} region amino acid sequence set forth in SEQ ID NO: 66.

In some embodiments, the V_{H} region of the antibody or antigen-binding fragment thereof comprise the amino acid sequence set forth in SEQ ID NOs: 63 and the V_{L} region of the antibody or antigen-binding fragment comprises the amino acid sequence set forth in SEQ ID NO: 66. In some embodiments, the V_{H} and V_{L} regions of the antibody or antigen-binding fragment thereof comprise the amino acid sequences set forth in SEQ ID NO: 63 and 66, respectively, or any antibody or antigen-binding fragment thereof that has at least 90% sequence identity to any of the above V_{H} and V_{L}, such as at least 90%, 91%, 92%, 93%, 94%, 95%, 96%, 97%, 98%, or 99% sequence identity thereto.

For example, the V_{H} and V_{L} regions of the antibody or antigen-binding fragment thereof provided therein comprise the amino acid sequence set forth in SEQ ID NO: 63 and 66, respectively.

Among the CARs provided herein is a CAR in which the antibody, such as an anti-CD19 antibody, or antibody fragment, in the provided CAR, comprises a V_{H} region amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NOs: 64, and a V_{L} region comprising an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 67.

In some embodiments, the V_{H} region of the antibody or antigen-binding fragment thereof comprises a CDR-H1, a CDR-H2, a CDR-H3, respectively, comprising the amino acid sequences of CDR-H1, CDR-H2, and CDR-H3 contained within the V_{H} region amino acid sequence set forth in SEQ ID NO: 64; and comprises a CDR-L1, a CDR-L2, a CDR-L3, respectively, comprising the amino acid sequences of CDR-L1, CDR-L2, and CDR-L3, respectively contained within the V_{L} region amino acid sequence set forth in SEQ ID NO: 67.

In some embodiments, the V_{H} region of the antibody or antigen-binding fragment thereof comprise the amino acid sequence set forth in SEQ ID NOs: 64 and the V_{L} region of the antibody or antigen-binding fragment comprises the amino acid sequence set forth in SEQ ID NO: 67. In some embodiments, the V_{H} and V_{L} regions of the antibody or antigen-binding fragment thereof comprise the amino acid sequences set forth in SEQ ID NO: 64 and 67, respectively, or any antibody or antigen-binding fragment thereof that has at least 90% sequence identity to any of the above V_{H} and V_{L}, such as at least 90%, 91%, 92%, 93%, 94%, 95%, 96%, 97%, 98%, or 99% sequence identity thereto.

For example, the V_{H} and V_{L} regions of the antibody or antigen-binding fragment thereof provided therein comprise the amino acid sequence set forth in SEQ ID NO: 64 and 67, respectively.

Among the CARs provided herein is a CAR in which the antibody, such as an anti-CD19 antibody, or antibody fragment, in the provided CAR, comprises a V_{H} region amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NOs: 64, and a V_{L} region comprising an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 68.

In some embodiments, the V_{H} region of the antibody or antigen-binding fragment thereof comprises a CDR-H1, a CDR-H2, a CDR-H3, respectively, comprising the amino acid sequences of CDR-H1, CDR-H2, and CDR-H3 contained within the V_{H} region amino acid sequence set forth in SEQ ID NO: 64; and comprises a CDR-L1, a CDR-L2, a CDR-L3, respectively, comprising the amino acid sequences of CDR-L1, CDR-L2, and CDR-L3, respectively contained within the V_{L} region amino acid sequence set forth in SEQ ID NO: 68.

In some embodiments, the V_{H} region of the antibody or antigen-binding fragment thereof comprise the amino acid sequence set forth in SEQ ID NOs: 64 and the V_{L} region of the antibody or antigen-binding fragment comprises the amino acid sequence set forth in SEQ ID NO: 68. In some embodiments, the V_{H} and V_{L} regions of the antibody or antigen-binding fragment thereof comprise the amino acid sequences set forth in SEQ ID NO: 64 and 68, respectively, or any antibody or antigen-binding fragment thereof that has at least 90% sequence identity to any of the above V_{H} and V_{L}, such as at least 90%, 91%, 92%, 93%, 94%, 95%, 96%, 97%, 98%, or 99% sequence identity thereto.

For example, the V_{H} and V_{L} regions of the antibody or antigen-binding fragment thereof provided therein comprise the amino acid sequence set forth in SEQ ID NO: 64 and 68, respectively.

Among the CARs provided herein is a CAR in which the antibody, such as an anti-CD19 antibody, or antibody fragment, in the provided CAR, comprises a V_{H} region amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NOs: 64, and a V_{L} region comprising an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 69.

In some embodiments, the V_{H} region of the antibody or antigen-binding fragment thereof comprises a CDR-H1, a CDR-H2, a CDR-H3, respectively, comprising the amino acid sequences of CDR-H1, CDR-H2, and CDR-H3 contained within the V_{H} region amino acid sequence set forth in SEQ ID NO: 64; and comprises a CDR-L1, a CDR-L2, a CDR-L3, respectively, comprising the amino acid sequences of CDR-L1, CDR-L2, and CDR-L3, respectively contained within the V_{L} region amino acid sequence set forth in SEQ ID NO: 69.

In some embodiments, the V_{H} region of the antibody or antigen-binding fragment thereof comprise the amino acid sequence set forth in SEQ ID NOs: 64 and the V_{L} region of the antibody or antigen-binding fragment comprises the amino acid sequence set forth in SEQ ID NO: 69. In some embodiments, the V_{H} and V_{L} regions of the antibody or antigen-binding fragment thereof comprise the amino acid sequences set forth in SEQ ID NO: 64 and 69, respectively, or any antibody or antigen-binding fragment thereof that has at least 90% sequence identity to any of the above V_{H} and V_{L}, such as at least 90%, 91%, 92%, 93%, 94%, 95%, 96%, 97%, 98%, or 99% sequence identity thereto.

For example, the V_{H} and V_{L} regions of the antibody or antigen-binding fragment thereof provided therein comprise the amino acid sequence set forth in SEQ ID NO: 64 and 69, respectively.

In some embodiments, the antibody or antigen-binding fragment thereof, in the provided CAR, is a single-chain antibody fragment, such as a single chain variable fragment (scFv) or a diabody or a single domain antibody (sdAb). In some embodiments, the antibody or antigen-binding fragment is a single domain antibody comprising only the V_{H} region. In some embodiments, the antibody or antigen binding fragment is based on an scFv comprising a heavy chain variable (V_{H}) region and a light chain variable (V_{L}) region. In some embodiments, the single-chain antibody fragment includes one or more linkers joining two antibody domains or regions, such as a heavy chain variable (V_{H}) region and a light chain variable (V_{L}) region. The linker typically is a peptide linker, e.g., a flexible and/or soluble peptide linker. Among the linkers are those rich in glycine and serine and/or in some cases threonine. In some embodiments, the linkers further include charged residues such as lysine and/or glutamate, which can improve solubility. In some embodiments, the linkers further include one or more proline.

Accordingly, the provided CARs contain anti-CD19 antibodies that include single-chain antibody fragments, such as scFvs and diabodies, particularly human single-chain antibody fragments, typically comprising linker(s) joining two antibody domains or regions, such V_{H} and V_{L} regions. The linker typically is a peptide linker, e.g., a flexible and/or soluble peptide linker, such as one rich in glycine and serine. In some embodiments, the CD19-binding domain comprises a linker between the V_{H} and V_{L} regions. In some embodiments, in order from N- to C-terminus, the CD19-binding domain comprises one of the V_{H} and V_{L} regions, a linker, and the other of the V_{H} and V_{L} regions. In some embodiments, the linker is set forth in SEQ ID NO: 59. Thus, in some embodiments, in order from N- to C-terminus, the CD19-binding domain comprises one of the V_{H} and V_{L} regions, the linker set forth in SEQ ID NO: 59, and the other of the V_{H} and V_{L} regions.

In some aspects, the linkers rich in glycine and serine (and/or threonine) include at least 80%, 85%, 90%, 91%, 92%, 93%, 94%, 95%, 96%, 97%, 98%, or 99% such amino acid(s). In some embodiments, they include at least at or about 50%, 55%, 60%, 70%, or 75%, glycine, serine, and/or threonine. In some embodiments, the linker is comprised substantially entirely of glycine, serine, and/or threonine. The linkers generally are between about 5 and about 50 amino acids in length, typically between at or about 10 and at or about 30, e.g., 10, 11, 12, 13, 14, 15, 16, 17, 18, 19, 20, 21, 22, 23, 24, 25, 26, 27, 28, 29, or 30, and in some examples between 10 and 25 amino acids in length. Exemplary linkers include linkers having various numbers of repeats of the sequence GGGGS (4GS; SEQ ID NO: 60), such as between 2, 3, 4 and 5 repeats of such a sequence. Exemplary linkers include those having or consisting of an sequence set forth in SEQ ID NO: 58 (GGGGSGGGGSGGGGS). Exemplary linkers further include those having or consisting of the sequence set forth in SEQ ID NO: 59 (GSTSGSGKPGSGEGSTKG), SEQ ID NO: 61 (GGGGSGGGGS), and SEQ ID NO: 62 (GGGGSGGGGSGGGGSGGGGS).

Accordingly, in some embodiments, the provided embodiments include single-chain antibody fragments, e.g., scFvs, comprising one or more of the aforementioned linkers, such as glycine/serine rich linkers, including linkers having repeats of GGGS (SEQ ID NO: 60), such as the linker set forth in SEQ ID NO: 58, 61, or 62.

In some embodiments, the CAR comprises a loop format. In some embodiments, the V_{H} or V_{L} region of the CD19-binding domain is joined to the V_{H} or the V_{L} region of the BAFF-R-binding domain by a linker. In some embodiments, one of the V_{H} and the V_{L} region of the CD19-binding domain is joined to the other of the V_{H} and the V_{L} region of the CD19-binding domain by a linker. In some embodiments, one of the V_{H} and the V_{L} region of the BAFF-R-binding domain is joined to the other of the V_{H} and the V_{L} region of the BAFF-R-binding domain by a linker. In some embodiments, the linker is set forth in SEQ ID NO: 58. In some embodiments, the linker is set forth in SEQ ID NO: 59. In some embodiments, the linker is set forth in SEQ ID NO: 60. In some embodiments, the linker is set forth in SEQ ID NO: 61. In some embodiments, the linker is set forth in SEQ ID NO: 62.

In some embodiments, the V_{H} region of the CD19-binding domain is joined to the V_{L} region of the BAFF-R-binding domain by the linker set forth in SEQ ID NO: 62. In some embodiments, the V_{H} region of the CD19-binding domain is joined to the V_{L} region of the BAFF-R-binding domain by the linker set forth in SEQ ID NO: 60. In some embodiments, the V_{H} region of the CD19-binding domain is joined to the V_{L} region of the BAFF-R-binding domain by the linker set forth in SEQ ID NO: 61. In some embodiments, the V_{H} region of the CD19-binding domain is joined to the V_{H} region of the BAFF-R-binding domain by the linker set forth in SEQ ID NO: 62. In some embodiments, the V_{H} region of the CD19-binding domain is joined to the V_{H} region of the BAFF-R-binding domain by the linker set forth in SEQ ID NO: 60. In some embodiments, the V_{H} region of the CD19-binding domain is joined to the V_{H} region of the BAFF-R-binding domain by the linker set forth in SEQ ID NO: 61.

In some embodiments, the V_{L} region of the CD19-binding domain is joined to the V_{L} region of the BAFF-R-binding domain by the linker set forth in SEQ ID NO: 62. In some embodiments, the V_{L} region of the CD19-binding domain is joined to the V_{L} region of the BAFF-R-binding domain by the linker set forth in SEQ ID NO: 60. In some embodiments, the V_{L} region of the CD19-binding domain is joined to the V_{L} region of the BAFF-R-binding domain by the linker set forth in SEQ ID NO: 61. In some embodiments, the V_{L} region of the CD19-binding domain is joined to the V_{H} region of the BAFF-R-binding domain by the linker set forth in SEQ ID NO: 62. In some embodiments, the V_{L} region of the CD19-binding domain is joined to the V_{H} region of the BAFF-R-binding domain by the linker set forth in SEQ ID NO: 60. In some embodiments, the V_{L} region of the CD19-binding domain is joined to the V_{H} region of the BAFF-R-binding domain by the linker set forth in SEQ ID NO: 61.

Accordingly, in some embodiments, the provided embodiments include single-chain antibody fragments, e.g., scFvs, comprising one or more of the aforementioned linkers, such as glycine/serine rich linkers, including linkers having repeats of GGGGS (SEQ ID NO: 58), such as the linker set forth in SEQ ID NO: 60, 61, or 62. In some embodiments, the linker comprises the sequence set forth in SEQ ID NO: 58. In some embodiments, the linker comprises the sequence set forth in SEQ ID NO: 60. In some embodiments, the linker comprises the sequence set forth in SEQ ID NO: 61. In some embodiments, the linker comprises the sequence set forth in SEQ ID NO: 62.

In some embodiments, the V_{H} region may be amino terminal to the V_{L} region. In some embodiments, the V_{H} region may be carboxy terminal to the V_{L} region. In particular embodiments, the fragment may include a V_{H} region or portion thereof, followed by the linker, followed by a V_{L} region or portion thereof. In other embodiments, the fragment may include the V_{L} region or portion thereof, followed by the linker, followed by the V_{H} region or portion thereof.

In some embodiments, the CAR comprises a linear format. Thus, in some embodiments, the CAR comprises a V_{H} and V_{L} of an anti-BAFF-R scFv and a V_{H} and V_{L} of an anti-CD19 scFv. In some embodiments, the V_{H} and V_{L} of the anti-BAFF-R scFv and the V_{H} and V_{L} of the anti-CD19 scFv are joined by a linker. In some embodiments, the linker is set forth in SEQ ID NO: 58. In some embodiments, the linker is set forth in SEQ ID NO: 60. In some embodiments, the linker is set forth in SEQ ID NO: 61. In some embodiments, the V_{H} and V_{L} regions of the anti-BAFF-R antibody are joined by the linker set forth in SEQ ID NO: 58 or 60-62. In some embodiments, the V_{H} and V_{L} regions of the anti-CD19 antibody are joined by the linker set forth in SEQ ID NO: 59.

In some aspects, a CD19 scFv provided herein comprises the amino acid sequence set forth in SEQ ID NOs: 119 or 70-74, or has an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NOs: 119 or 70-74. In some aspects, an scFv provided herein comprises the amino acid sequence set forth in SEQ ID NO: 119, or has an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 119. In some aspects, an scFv provided herein comprises the amino acid sequence set forth in SEQ ID NO: 119. In some aspects, an scFv provided herein comprises an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 119.

In some aspects, an scFv provided herein comprises the amino acid sequence set forth in SEQ ID NO: 70, or has an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 70. In some aspects, an scFv provided herein comprises the amino acid sequence set forth in SEQ ID NO: 70. In some aspects, an scFv provided herein comprises an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 70.

In some aspects, an scFv provided herein comprises the amino acid sequence set forth in SEQ ID NO: 71, or has an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 71. In some aspects, an scFv provided herein comprises the amino acid sequence set forth in SEQ ID NO: 71. In some aspects, an scFv provided herein comprises an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 71.

In some aspects, an scFv provided herein comprises the amino acid sequence set forth in SEQ ID NO: 72, or has an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 72. In some aspects, an scFv provided herein comprises the amino acid sequence set forth in SEQ ID NO: 72. In some aspects, an scFv provided herein comprises an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 72.

In some aspects, an scFv provided herein comprises the amino acid sequence set forth in SEQ ID NO: 73, or has an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 73. In some aspects, an scFv provided herein comprises the amino acid sequence set forth in SEQ ID NO: 73. In some aspects, an scFv provided herein comprises an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 73.

In some aspects, an scFv provided herein comprises the amino acid sequence set forth in SEQ ID NO: 74, or has an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 74. In some aspects, an scFv provided herein comprises the amino acid sequence set forth in SEQ ID NO: 74. In some aspects, an scFv provided herein comprises an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 74.

Among the provided CARs is a CAR in which the CD19-binding domain contains a V_{H} region comprising the sequence set forth in SEQ ID NO: 41 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO: 41; and contains a V_{L} region comprising the sequence set forth in SEQ ID NO: 42 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO: 42. In some embodiments, the CD19-binding domain of the provided CAR contains a V_{H} region that has a CDRH1, a CDRH2 and a CDRH3 comprising the amino acid sequence of SEQ ID NOS: 43, 44, and 45, respectively, or 43, 44, and 46 respectively, and a V_{L} region that has a CDRL1, a CDRL2 and a CDRL3 comprising the amino acid sequence of SEQ ID NOS: 47, 48, and 50, respectively; 47, 49, and 50, respectively; 47, 48, and 51, respectively; or 47, 49, and 51, respectively. In some embodiments, the V_{H} region comprises the sequence set forth in SEQ ID NO: 41 and the V_{L} region comprises the sequence set forth in SEQ ID NO: 42.

Among the provided CARs is a CAR in which the CD19-binding domain contains a V_{H} region comprising the sequence set forth in SEQ ID NO: 63 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO: 63; and contains a V_{L} region comprising the sequence set forth in SEQ ID NO: 65 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO: 65. In some embodiments, the CD19-binding domain of the provided CAR contains a V_{H} region that has a CDRH1, a CDRH2 and a CDRH3 comprising the amino acid sequence of SEQ ID NOS: 75, 76, and 77, respectively, and a V_{L} region that has a CDRL1, a CDRL2 and a CDRL3 comprising the amino acid sequence of SEQ ID NOS: 78, 79, and 80. In some embodiments, the V_{H} region comprises the sequence set forth in SEQ ID NO: 63 and the V_{L} region comprises the sequence set forth in SEQ ID NO: 65.

Among the provided CARs is a CAR in which the CD19-binding domain contains a V_{H} region comprising the sequence set forth in SEQ ID NO: 63 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO: 63; and contains a V_{L} region comprising the sequence set forth in SEQ ID NO: 66 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO: 66. In some embodiments, the CD19-binding domain of the provided CAR contains a V_{H} region that has a CDRH1, a CDRH2 and a CDRH3 comprising the amino acid sequence of SEQ ID NOS: 75, 76, and 77, respectively, and a V_{L} region that has a CDRL1, a CDRL2 and a CDRL3 comprising the amino acid sequence of SEQ ID NOS: 81, 82, and 83, respectively. In some embodiments, the V_{H} region comprises the sequence set forth in SEQ ID NO: 63 and the V_{L} region comprises the sequence set forth in SEQ ID NO: 66.

Among the provided CARs is a CAR in which the CD19-binding domain contains a V_{H} region comprising the sequence set forth in SEQ ID NO: 64 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO: 64; and contains a V_{L} region comprising the sequence set forth in SEQ ID NO: 67 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO: 67. In some embodiments, the CD19-binding domain of the provided CAR contains a V_{H} region that has a CDRH1, a CDRH2 and a CDRH3 comprising the amino acid sequence of SEQ ID NOS: 75, 76, and 77, respectively, and a V_{L} region that has a CDRL1, a CDRL2 and a CDRL3 comprising the amino acid sequence of SEQ ID NOS: 84, 85, and 86, respectively. In some embodiments, the V_{H} region comprises the sequence set forth in SEQ ID NO: 64 and the V_{L} region comprises the sequence set forth in SEQ ID NO: 67.

Among the provided CARs is a CAR in which the CD19-binding domain contains a V_{H} region comprising the sequence set forth in SEQ ID NO: 64 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO: 64; and contains a V_{L} region comprising the sequence set forth in SEQ ID NO: 68 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO: 68. In some embodiments, the CD19-binding domain of the provided CAR contains a V_{H} region that has a CDRH1, a CDRH2 and a CDRH3 comprising the amino acid sequence of SEQ ID NOS: 75, 76, and 77, respectively, and a V_{L} region that has a CDRL1, a CDRL2 and a CDRL3 comprising the amino acid sequence of SEQ ID NOS: 84, 85, and 87. In some embodiments, the V_{H} region comprises the sequence set forth in SEQ ID NO: 64 and the V_{L} region comprises the sequence set forth in SEQ ID NO: 68.

Among the provided CARs is a CAR in which the CD19-binding domain contains a V_{H} region comprising the sequence set forth in SEQ ID NO: 64 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO: 64; and contains a V_{L} region comprising the sequence set forth in SEQ ID NO: 69 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO: 69. In some embodiments, the CD19-binding domain of the provided CAR contains a V_{H} region that has a CDRH1, a CDRH2 and a CDRH3 comprising the amino acid sequence of SEQ ID NOS: 75, 76, and 77, respectively, and a V_{L} region that has a CDRL1, a CDRL2 and a CDRL3 comprising the amino acid sequence of SEQ ID NOS: 88, 89, and 90, respectively. In some embodiments, the V_{H} region comprises the sequence set forth in SEQ ID NO: 64 and the V_{L} region comprises the sequence set forth in SEQ ID NO: 69.

Among the antibodies, e.g., antigen-binding fragments, in the provided CARs, are human antibodies. In some embodiments of a provided human anti-CD19 antibody, e.g., antigen-binding fragments, the human antibody contains a V_{H} region that comprises a portion having at least 95%, 96%, 97%, 98%, 99%, or 100% sequence identity to an amino acid sequence encoded by a germline nucleotide human heavy chain V segment, a portion having at least 95%, 96%, 97%, 98%, 99%, or 100% sequence identity to an amino acid sequence encoded by a germline nucleotide human heavy chain D segment, and/or a portion having at least 95%, 96%, 97%, 98%, 99%, or 100% sequence identity to an amino acid sequence encoded by a germline nucleotide human heavy chain J segment; and/or contains a V_{L} region that comprises a portion having at least 95%, 96%, 97%, 98%, 99%, or 100% sequence identity to an amino acid sequence encoded by a germline nucleotide human kappa or lambda chain V segment, and/or a portion having at least 95%, 96%, 97%, 98%, 99%, or 100% sequence identity to an amino acid sequence encoded by a germline nucleotide human kappa or lambda chain J segment. In some embodiments, the portion of the V_{H} region corresponds to the CDR-H1, CDR-H2 and/or CDR-H3. In some embodiments, the portion of the V_{H} region corresponds to the framework region 1 (FR1), FR2, FR2 and/or FR4. In some embodiments, the portion of the V_{L} region corresponds to the CDR-L1, CDR-L2 and/or CDR-L3. In some embodiments, the portion of the V_{L} region corresponds to the FR1, FR2, FR2 and/or FR4.

In some embodiments, the human antibody, e.g., antigen-binding fragment, contains a CDR-H1 having at least 95%, 96%, 97%, 98%, 99%, or 100% sequence identity to an amino acid sequence of the corresponding CDR-H1 region within a sequence encoded by a germline nucleotide human heavy chain V segment. For example, the human antibody in some embodiments contains a CDR-H1 having a sequence that is 100% identical or with no more than one, two or three amino acid differences as compared to the corresponding CDR-H1 region within a sequence encoded by a germline nucleotide human heavy chain V segment.

In some embodiments, the human antibody, e.g., antigen-binding fragment, contains a CDR-H2 having at least 95%, 96%, 97%, 98%, 99%, or 100% sequence identity to an amino acid sequence of the corresponding CDR-H2 region within a sequence encoded by a germline nucleotide human heavy chain V segment. For example, the human antibody in some embodiments contains a CDR-H2 having a sequence that is 100% identical or with no more than one, two or three amino acid difference as compared to the corresponding CDR-H2 region within a sequence encoded by a germline nucleotide human heavy chain V segment.

In some embodiments, the human antibody, e.g., antigen-binding fragment, contains a CDR-H3 having at least 95%, 96%, 97%, 98%, 99%, or 100% sequence identity to an amino acid sequence of the corresponding CDR-H3 region within a sequence encoded by a germline nucleotide human heavy chain V segment, D segment and J segment. For example, the human antibody in some embodiments contains a CDR-H3 having a sequence that is 100% identical or with no more than one, two or three amino acid differences as compared to the corresponding CDR-H3 region within a sequence encoded by a germline nucleotide human heavy chain V segment, D segment and J segment.

In some embodiments, the human antibody, e.g., antigen-binding fragment, contains a CDR-L1 having at least 95%, 96%, 97%, 98%, 99%, or 100% sequence identity to an amino acid sequence of the corresponding CDR-L1 region within a sequence encoded by a germline nucleotide human light chain V segment. For example, the human antibody in some embodiments contains a CDR-L1 having a sequence that is 100% identical or with no more than one, two or three amino acid differences as compared to the corresponding CDR-L1 region within a sequence encoded by a germline nucleotide human light chain V segment.

In some embodiments, the human antibody, e.g., antigen-binding fragment, contains a CDR-L2 having at least 95%, 96%, 97%, 98%, 99%, or 100% sequence identity to an amino acid sequence of the corresponding CDR-L2 region within a sequence encoded by a germline nucleotide human light chain V segment. For example, the human antibody in some embodiments contains a CDR-L2 having a sequence that is 100% identical or with no more than one, two or three amino acid difference as compared to the corresponding CDR-L2 region within a sequence encoded by a germline nucleotide human light chain V segment.

In some embodiments, the human antibody, e.g., antigen-binding fragment, contains a CDR-L3 having at least 95%, 96%, 97%, 98%, 99%, or 100% sequence identity to an amino acid sequence of the corresponding CDR-L3 region within a sequence encoded by a germline nucleotide human light chain V segment and J segment. For example, the human antibody in some embodiments contains a CDR-L3 having a sequence that is 100% identical or with no more than one, two or three amino acid differences as compared to the corresponding CDR-L3 region within a sequence encoded by a germline nucleotide human light chain V segment and J segment.

In some embodiments, the human antibody, e.g., antigen-binding fragment, contains a framework region that contains human germline gene segment sequences. For example, in some embodiments, the human antibody contains a V_{H} region in which the framework region, e.g. FR1, FR2, FR3 and FR4, has at least 95%, 96%, 97%, 98%, 99%, or 100% sequence identity to a framework region encoded by a human germline antibody segment, such as a V segment and/or J segment. In some embodiments, the human antibody contains a V_{L} region in which the framework region e.g. FR1, FR2, FR3 and FR4, has at least 95%, 96%, 97%, 98%, 99%, or 100% sequence identity to a framework region encoded by a human germline antibody segment, such as a V segment and/or J segment. For example, in some such embodiments, the framework region sequence contained within the V_{H} region and/or V_{L} region differs by no more than 10 amino acids, such as no more than 9, 8, 7, 6, 5, 4, 3, 2 or 1 amino acid, compared to the framework region sequence encoded by a human germline antibody segment.

c. Orientation of Extracellular Binding Domain

In some embodiments, the extracellular binding domain comprises, in any order, the V_{H} region of a BAFF-R-binding domain, the V_{L} region of a BAFF-R-binding domain, the V_{H} region of a CD19-binding domain, and the V_{L} of a CD19-binding domain. In some embodiments, the extracellular binding domain comprises, from amino to carboxy terminus: a V_{H} region of the BAFF-R-binding domain, a V_{L} region of the CD19-binding domain, a V_{H} region of the CD19-binding domain, and a V_{L} region of the BAFF-R-binding domain. In some embodiments, the extracellular binding domain comprises, from amino to carboxy terminus: a V_{L} region of the BAFF-R-binding domain, a V_{L} region of the CD19-binding domain, a V_{H} region of the CD19-binding domain, and a V_{H} region of the BAFF-R-binding domain. In some embodiments, the extracellular binding domain comprises, from amino to carboxy terminus: a V_{L} region of the CD19-binding domain, a V_{H} region of the BAFF-R-binding domain, a V_{L} region of the BAFF-R-binding domain, and a Vu region of the CD19-binding domain.

In some embodiments, the extracellular binding domain comprises, from amino to carboxy terminus: a V_{H} of the CD19-binding domain, a V_{H} region of the BAFF-R-binding domain, a V_{L} region of the BAFF-R-binding domain, and a V_{L} region of the CD19-binding domain. In some embodiments, the extracellular binding domain comprises, from amino to carboxy terminus: a V_{L} region of the CD19-binding domain, a V_{H} region of the CD19-binding domain, a V_{H} region of the BAFF-R-binding domain, and a V_{L} region of the BAFF-R-binding domain. In some embodiments, the extracellular binding domain comprises, from amino to carboxy terminus: a V_{L} region of the CD19-binding domain, a V_{H} region of the CD19-binding domain, a V_{L} region of the BAFF-R-binding domain, and a V_{H} region of the BAFF-R-binding domain.

In some embodiments, the extracellular binding domain comprises, from amino to carboxy terminus: a V_{L} region that is or comprises the amino acid sequence encoded by SEQ ID NO: 42 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 42; a V_{H} region that is or comprises the amino acid sequence encoded by SEQ ID NO: 1 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 1; a V_{L} region that is or comprises the amino acid sequence encoded by SEQ ID NO: 2 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 2; and a V_{H} region that is or comprises the amino acid sequence encoded by SEQ ID NO: 41 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 41.

In some embodiments, the extracellular binding domain comprises, from amino to carboxy terminus: a V_{L} region that is or comprises the amino acid sequence encoded by SEQ ID NO: 42 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 42; a V_{H} region that is or comprises the amino acid sequence encoded by SEQ ID NO: 3 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 3; a V_{L} region that is or comprises the amino acid sequence encoded by SEQ ID NO: 4 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 4; and a V_{H} region that is or comprises the amino acid sequence encoded by SEQ ID NO: 41 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 41.

In some embodiments, the extracellular binding domain comprises, from amino to carboxy terminus: a V_{L} region that is or comprises the amino acid sequence encoded by SEQ ID NO: 4 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 4; a V_{L} region that is or comprises the amino acid sequence encoded by SEQ ID NO: 42 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 42; a V_{H} region that is or comprises the amino acid sequence encoded by SEQ ID NO: 41 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 41; and a V_{H} region that is or comprises the amino acid sequence encoded by SEQ ID NO: 3 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 3.

In some embodiments, the extracellular binding domain comprises, from amino to carboxy terminus: a V_{H} region that is or comprises the amino acid sequence encoded by SEQ ID NO: 3 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 3; a V_{L} region that is or comprises the amino acid sequence encoded by SEQ ID NO: 42 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 42; a V_{H} region that is or comprises the amino acid sequence encoded by SEQ ID NO: 41 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 41; and a V_{L} region that is or comprises the amino acid sequence encoded by SEQ ID NO: 4 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 4.

In some embodiments, the extracellular binding domain comprises, from amino to carboxy terminus: a V_{L} region that is or comprises the amino acid sequence encoded by SEQ ID NO: 6 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 6; a V_{L} region that is or comprises the amino acid sequence encoded by SEQ ID NO: 42 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 42; a V_{H} region that is or comprises the amino acid sequence encoded by SEQ ID NO: 41 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 41; and a V_{H} region that is or comprises the amino acid sequence encoded by SEQ ID NO: 5 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 5.

In some embodiments, the extracellular binding domain comprises, from amino to carboxy terminus: a V_{L} region that is or comprises the amino acid sequence encoded by SEQ ID NO: 42 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 42; a V_{H} region that is or comprises the amino acid sequence encoded by SEQ ID NO: 9 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 9; a V_{L} region that is or comprises the amino acid sequence encoded by SEQ ID NO: 10 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 10; and a V_{H} region that is or comprises the amino acid sequence encoded by SEQ ID NO: 41 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 41.

In some embodiments, the extracellular binding domain comprises, from amino to carboxy terminus: a V_{L} region that is or comprises the amino acid sequence encoded by SEQ ID NO: 42 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 42; a V_{H} region that is or comprises the amino acid sequence encoded by SEQ ID NO: 41 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 41; a V_{H} region that is or comprises the amino acid sequence encoded by SEQ ID NO: 3 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 3; and a V_{L} region that is or comprises the amino acid sequence encoded by SEQ ID NO: 4 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 4.

In some embodiments, the extracellular binding domain comprises, from amino to carboxy terminus: a V_{L} region that is or comprises the amino acid sequence encoded by SEQ ID NO: 42 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 42; a V_{H} region that is or comprises the amino acid sequence encoded by SEQ ID NO: 41 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 41; a V_{L} region that is or comprises the amino acid sequence encoded by SEQ ID NO: 4 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 4; and a V_{H} region that is or comprises the amino acid sequence encoded by SEQ ID NO: 3 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 3.

In some embodiments, the extracellular binding domain comprises, from amino to carboxy terminus: a V_{L} region that is or comprises the amino acid sequence encoded by SEQ ID NO: 42 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 42; a V_{H} region that is or comprises the amino acid sequence encoded by SEQ ID NO: 41 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 41; a V_{H} region that is or comprises the amino acid sequence encoded by SEQ ID NO: 7 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 7; and a V_{L} region that is or comprises the amino acid sequence encoded by SEQ ID NO: 8 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 8.

In some embodiments, the extracellular binding domain comprises, from amino to carboxy terminus: a V_{L} region that is or comprises the amino acid sequence encoded by SEQ ID NO: 42 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 42; a V_{H} region that is or comprises the amino acid sequence encoded by SEQ ID NO: 41 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 41; a V_{L} region that is or comprises the amino acid sequence encoded by SEQ ID NO: 4 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 4; and a V_{H} region that is or comprises the amino acid sequence encoded by SEQ ID NO: 3 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 3.

### B. Spacer

In some embodiments, the recombinant receptor such as a CAR comprising an antibody (e.g., antigen-binding fragment) provided herein further include a spacer domain (in some cases also called a spacer region). In some embodiments, the spacer is or includes at least a portion of an immunoglobulin constant region or variant or modified version thereof. In some embodiments, the portion of the immunoglobulin constant region includes a hinge region, e.g., an IgG4 hinge region, and/or a C_{H}1, C_{H}2 or C_{H}3 and/or Fc region. In some embodiments, the constant region or portion is of a human IgG, such as IgG4 or IgG1. In some aspects, the portion of the constant region serves as a spacer region between the antigen-binding domain or a portion thereof (e.g., a V_{H} or V_{L} of the BAFF-R-binding domain or the CD19-binding domain) and transmembrane domain (also referred to herein as a transmembrane region).

In some embodiments, the spacer is or includes at least a portion of human CD4, CD8, or CD28 proteins. In some embodiments, the spacer is or includes a hinge region from CD4, CD8, or CD28 extracellular domains.

In some embodiments, the length of the spacer is adjusted to optimize the biophysical synapse distance between the CAR-expressing cell and the target of the CAR, such as a CAR-expressing T-cell, and the target of the CAR, such as a BAFF-R-expressing or CD19-expressing cell. In some embodiments, the CAR is expressed by a T cell, and the length of the spacer is adjusted to a length that is compatible for T cell activation or to optimize CAR T-cell performance.

In some embodiments, the spacer can be of a length that provides for increased responsiveness of the cell following antigen binding, as compared to in the absence of the spacer or as compared to an alternative spacer of a different length (e.g. longer in length). In some examples, the spacer is at or about 12 to 15 amino acids in length. In some examples, the spacer is at or about 220 to 240 amino acids in length.

Exemplary spacers include those having at least at or about 10 to at or about 300 amino acids, at or about 10 to at or about 229 amino acids, at or about 10 to at or about 200 amino acids, at or about 10 to at or about 175 amino acids, at or about 10 to at or about 150 amino acids, at or about 10 to at or about 125 amino acids, at or about 10 to at or about 100 amino acids, at or about 10 to at or about 75 amino acids, at or about 10 to at or about 50 amino acids, at or about 10 to at or about 40 amino acids, at or about 10 to at or about 30 amino acids, at or about 10 to at or about 20 amino acids, or at or about 12 to at or about 15 amino acids in length, and including any integer between the endpoints of any of the listed ranges. Exemplary spacers include those having at least at or about at or about 50 to at or about 175 amino acids, at or about 50 to at or about 150 amino acids, at or about 10 to at or about 125 amino acids, at or about 50 to at or about 100 amino acids, at or about 100 to at or about 300 amino acids, at or about 100 to at or about 250 amino acids, at or about 125 to at or about 250 amino acids, or at or about 200 to at or about 250 amino acids, and including any integer between the endpoints of any of the listed ranges. In some embodiments, a spacer is at least at or about 12 amino acids, at least at or about 119 amino acids, at least at or about 125 amino acids, at least at or about 200 amino acids, or at least at or about 220 amino acids, or at least at or about 225 amino acids in length. In some embodiments, a spacer is at least at or about 13 amino acids, at least at or about 120 amino acids, at least at or about 125 amino acids, at least at or about 200 amino acids, or at least at or about 220 amino acids, or at least at or about 229 amino acids in length. In some embodiments, a spacer is at or about 11, 12, 13, 14, 15, 16, 17, 18, 19, 20, 21, 22, 23, 24, 25 amino acids or less in length. In some embodiments, the spacer is at least at or about 100 amino acids in length, such as at least at or about 110, 111, 112, 113, 114, 115, 116, 117, 118, 119, 120, 121, 122, 123, 124, 125, 130, 135, 140, 145, 150, 160, 170, 180, 190, 200, 210, 220, 230, 240, or 250 amino acids in length. In some embodiments, the spacer is between 220 and 240 amino acids in length.

In some embodiments, the spacer is at least at or about 125 to at or about 300 amino acids, at or about 125 to at or about 250 amino acids, at or about 125 to at or about 230 amino acids, at or about 125 to at or about 200 amino acids, at or about 125 to at or about 180 amino acids, at or about 125 to at or about 150 amino acids, at or about 150 to at or about 300 amino acids, at or about 150 to at or about 250 amino acids, at or about 150 to at or about 230 amino acids, at or about 150 to at or about 200 amino acids, at or about 150 to at or about 180 amino acids, at or about 180 to at or about 300 amino acids, at or about 180 to at or about 250 amino acids, at or about 180 to at or about 230 amino acids, at or about 180 to at or about 200 amino acids, at or about 200 to at or about 300 amino acids, at or about 200 to at or about 250 amino acids, at or about 200 to at or about 230 amino acids, at or about 230 to at or about 300 amino acids, at or about 230 to at or about 250 amino acids in length or 250 to at or about 300 amino acids in length.

Exemplary spacers include an IgG hinge alone, an IgG hinge linked to one or more of a C_{H}2 and C_{H}3 domain, IgG hinge linked to the C_{H}3 domain. In some embodiments, the spacer includes an IgG hinge alone. In some embodiments, the IgG hinge, C_{H}2 and/or C_{H}3 can be derived all or in part from IgG4 or IgG2, such as all or in part from human IgG4 or human IgG2. In some embodiments, the spacer can be a chimeric polypeptide containing one or more of a hinge, C_{H}2 and/or C_{H}3 sequence(s) derived from IgG4, IgG2, and/or IgG2 and IgG4. In some embodiments, the hinge region comprises all or a portion of an IgG4 hinge region. In some embodiments, the hinge region comprises all or a portion of an IgG4 hinge region and/or of an IgG2 hinge region, wherein the IgG4 hinge region is optionally a human IgG4 hinge region and the IgG2 hinge region is optionally a human IgG2 hinge region; the C_{H}2 region comprises all or a portion of an IgG4 C_{H}2 region and/or of an IgG2 C_{H}2 region, wherein the IgG4 C_{H}2 region is optionally a human IgG4 C_{H}2 region and the IgG2 C_{H}2 region is optionally a human IgG2 C_{H}2 region; and/or the C_{H}3 region comprises all or a portion of an IgG4 C_{H}3 region and/or of an IgG2 C_{H}3 region, wherein the IgG4 C_{H}3 region is optionally a human IgG4 C_{H}3 region and the IgG2 C_{H}3 region is optionally a human IgG2 C_{H}3 region. In some embodiments, the hinge, C_{H}2 and C_{H}3 comprises all or a portion of each of a hinge region, C_{H}2 and C_{H}3 from IgG4. In some embodiments, the hinge region is chimeric and comprises a hinge region from human IgG4 and human IgG2; the C_{H}2 region is chimeric and comprises a C_{H}2 region from human IgG4 and human IgG2; and/or the C_{H}3 region is chimeric and comprises a C_{H}3 region from human IgG4 and human IgG2. In some embodiments, the spacer comprises an IgG4/2 chimeric hinge or a modified IgG4 hinge comprising at least one amino acid replacement compared to human IgG4 hinge region; an human IgG2/4 chimeric C_{H}2 region; and a human IgG4 C_{H}3 region.

In some embodiments, the spacer and can contain one or more single amino acid mutations in one or more domains of the immunoglobulin, such as in one or more domains of the hinge, C_{H}2 or C_{H}3 region. In some examples, the amino acid modification is a substitution of a proline (P) for a serine(S) in the hinge region of an IgG4. In some embodiments, the spacer is or contains a IgG4 hinge that is a variant IgG4 hinge region comprising substitution of amino acids CPSC to CPPC compared to the wild-type IgG4 hinge region. In some embodiments, the spacer contains a C_{H}2 of an IgG4 that is a variant C_{H}2 in which amino acid modification is a substitution of a glutamine (Q) for an asparagine (N) to reduce glycosylation heterogeneity, such as an N177Q mutation at position 177, in the CH2 region, of the full-length IgG4 Fc sequence set forth in SEQ ID NO: 93 or an N176Q at position 176, in the C_{H}2 region, of the full-length IgG2 Fc sequence set forth in SEQ ID NO: 92.

In some examples, the spacer is at or about 12 amino acids in length or is no more than at or about 12 amino acids in length. In some examples, the spacer is at or about 15 amino acids in length or is no more than at or about 15 amino acids in length.

In some embodiments, the spacer comprises or consists of all or a portion of an immunoglobulin hinge or a modified version thereof. In some embodiments, the spacer is at or about 15 amino acids or less in length. In some embodiments, the spacer comprises or consists of all or a portion of an immunoglobulin hinge, optionally an IgG4 hinge, or a modified version thereof and/or comprises about 15 amino acids or less. In some embodiments, the spacer is at or about 13 amino acids in length and/or comprises or consists of all or a portion of an immunoglobulin hinge, optionally an IgG4, or a modified version thereof. In some embodiments, the spacer is at or about 12 amino acids in length and/or comprises or consists of all or a portion of an immunoglobulin hinge, optionally an IgG4, or a modified version thereof. In some embodiments, the spacer comprises the formula X₁PPX₂P (SEQ ID NO: 91), where X₁ is glycine, cysteine or arginine and X₂ is cysteine or threonine. In some embodiments, the spacer does not comprise a CD28 extracellular region or a CD8 extracellular region. In certain cases, the spacer has a methionine residue at the C-terminus. In some embodiments, the spacer comprises or consists of the sequence of SEQ ID NO: 52, or a variant of any of the foregoing having at least 85%, 86%, 87%, 88%, 89%, 90%, 91%, 92%, 93%, 94%, 95%, 96%, 97%, 98%, 99% or more sequence identity thereto.

In some embodiments, the spacer can be from all or in part from IgG4 and/or IgG2 and can contain mutations, such as one or more single amino acid mutations in one or more domains. In some examples, the amino acid modification is a substitution of a proline (P) for a serine(S) in the hinge region of an IgG4. In some embodiments, the spacer is or contains a IgG4 hinge that is a variant IgG4 hinge region comprising substitution of amino acids CPSC to CPPC compared to the wild-type IgG4 hinge region. In some embodiments, the amino acid modification is a substitution of a glutamine (Q) for an asparagine (N) to reduce glycosylation heterogeneity, such as an N177Q mutation at position 177, in the C_{H}2 region, of the full-length IgG4 Fc sequence set forth in SEQ ID NO: 93 or an N176Q at position 176, in the C_{H}2 region, of the full-length IgG2 Fc sequence set forth in SEQ ID NO: 92.

In some embodiments, the spacer is or comprises an IgG4/2 chimeric hinge or a modified IgG4 hinge; an IgG2/4 chimeric C_{H}2 region; and an IgG4 C_{H}3 region and optionally is about 228 or 229 amino acids in length; or a spacer set forth in SEQ ID NO: 53. In some embodiments, the spacer is or contains an amino acid sequence having at least at or about 85%, 86%, 87%, 88%, 89%, 90%, 91%, 92%, 93%, 94%, 95%, 96%, 97%, 98% or 99% identity to SEQ ID NO: 53. In some embodiments, the spacer is or contains the sequence set forth in SEQ ID NO: 53.

Additional exemplary spacers include, but are not limited to, those described in Hudecek et al. (2013) Clin. Cancer Res., 19:3153, Hudecek et al. (2015) Cancer Immunol. Res., 3(2): 125-135, or WO2014031687. In some embodiments, the nucleotide sequence of the spacer is optimized to reduce RNA heterogeneity upon expression. In some embodiments, the nucleotide sequence of the spacer is optimized to reduce cryptic splice sites or reduce the likelihood of a splice event at a splice site.

In some embodiments, the spacer has an amino acid sequence that exhibits at least 85%, 86%, 87%, 88%, 89%, 90%, 91%, 92%, 93%, 94%, 95%, 96%, 97%, 98%, 99% or more sequence identity to SEQ ID NO: 52 or 53, encoded by a polynucleotide that has been optionally optimized for codon usage and/or to reduce RNA heterogeneity. Methods to reduce RNA heterogeneity, such as by removing cryptic splice donor and/or acceptor sites, are described below. Observations have shown that cryptic splice donor and/or acceptor sites are present in the spacer region of certain immunoglobulin spacers when present in a CAR. In some embodiments, the spacer in a provided CAR is encoded by a polynucleotide in which one or more cryptic splice donor and/or acceptor sites are eliminated and/or are modified to reduce heterogeneity of the RNA transcribed from the construct, such as mRNA, following expression in a cell.

### C. Transmembrane Domain

The antigen-recognition component (i.e., the BAFF-R- and CD19-binding domains) generally is linked to one or more intracellular signaling components, such as signaling components that mimic activation through an antigen receptor complex, such as a TCR complex, in the case of a CAR, and/or signal via another cell surface receptor. Thus, in some embodiments, a BAFF-R-binding domain or a component thereof, or a CD19-binding domain or a component thereof, (e.g., antibody or antigen binding fragment thereof) is linked to one or more transmembrane domains such as those described herein and intracellular signaling domains comprising one or more intracellular components such as those described herein. In some embodiments, the V_{H} or the V_{L} of the binding domain most proximal to the cellular membrane is linked to the transmembrane domain. In some embodiments, the transmembrane domain is fused to the extracellular binding domain. In one embodiment, a transmembrane domain that naturally is associated with one of the domains in the receptor, e.g., CAR, is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane domains include those derived from (i.e. comprise at least the transmembrane domain(s) of) the alpha, beta or zeta chain of the T-cell receptor, CD3 epsilon, CD4, CD5, CD8, CD9, CD16, CD22, CD28, CD33, CD37, CD45, CD64, CD80, CD86, CD134, CD137, and/or CD154. For example, the transmembrane domain can be a CD4 transmembrane. In some embodiments, the transmembrane domain of the receptor is a transmembrane domain of human CD4 or variant thereof. In other embodiments, the transmembrane domain can be a CD8 transmembrane domain. In some embodiments, the transmembrane domain of the receptor is a transmembrane domain of human CD8 or variant thereof. In other embodiments, the transmembrane domain can be a CD28 transmembrane domain that comprises the sequence of amino acids set forth in SEQ ID NO: 55. In some embodiments, the transmembrane domain of the receptor is a transmembrane domain of human CD28 or variant thereof, e.g., a 27-amino acid transmembrane domain of a human CD28 (Accession No.: P10747.1), or a 28-amino acid sequence, or is a transmembrane domain that comprises the sequence of amino acids set forth in SEQ ID NO: 55 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 55.

In some embodiments, the transmembrane domain is or contains SEQ ID NO: 55 or an amino acid sequence having at least at or about 90%, 91%, 92%, 93%, 94%, 95%, 96%, 97%, 98% or 99% sequence identity to SEQ ID NO: 55. In some embodiments, the transmembrane domain is or contains the sequence set forth in SEQ ID NO: 55.

Alternatively, the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain. In some embodiments, the linkage is by linkers, spacers, and/or transmembrane domain(s).

### D. Intracellular Signaling Domain

Among the intracellular signaling domains are those that mimic or approximate a signal through a natural antigen receptor, a signal through such a receptor in combination with a costimulatory receptor, and/or a signal through a costimulatory receptor alone. In some embodiments, a short oligo- or polypeptide linker, for example, a linker of between 2 and 10 amino acids in length, such as one containing glycines and serines, e.g., glycine-serine doublet, is present and forms a linkage between the transmembrane domain and the intracellular signaling domain of the CAR.

The receptor, e.g., the CAR, generally includes an intracellular signaling region comprising at least one intracellular signaling component or components. In some embodiments, the receptor includes an intracellular component or signaling domain of a TCR complex, such as a TCR CD3 chain that mediates T-cell activation and cytotoxicity, e.g., CD3 zeta (CD3ζ) chain. Thus, in some aspects, the BAFF-R or CD19 binding domain is linked to one or more cell signaling modules. In some embodiments, cell signaling modules include CD3 intracellular signaling domains and/or other CD transmembrane domains. In some embodiments, the receptor, e.g., CAR, further includes a portion of one or more additional molecules such as Fc receptor γ, CD8, CD4, CD25, or CD16.

In some embodiments, upon ligation of the CAR, the cytoplasmic domain or intracellular signaling region of the CAR stimulates and/or activates at least one of the normal effector functions or responses of the immune cell, e.g., T cell engineered to express the CAR. For example, in some contexts, the CAR induces a function of a T cell such as cytolytic activity or T-helper activity, such as secretion of cytokines or other factors. In some embodiments, a truncated portion of an intracellular signaling domain of an antigen receptor component or costimulatory molecule is used in place of an intact immunostimulatory chain, for example, if it transduces the effector function signal. In some embodiments, the intracellular signaling domain or domains include the cytoplasmic sequences of the T cell receptor (TCR), and in some aspects also those of co-receptors that in the natural context act in concert with such receptor to initiate signal transduction following antigen receptor engagement, and/or any derivative or variant of such molecules, and/or any synthetic sequence that has the same functional capability.

In the context of a natural TCR, full activation generally requires not only signaling through the TCR, but also a costimulatory signal. Thus, in some embodiments, to promote full activation, a component for generating secondary or co-stimulatory signal is also included in the CAR. In other embodiments, the CAR does not include a component for generating a costimulatory signal. In some aspects, an additional CAR is expressed in the same cell and provides the component for generating the secondary or costimulatory signal.

T cell activation is in some aspects described as being mediated by two classes of cytoplasmic signaling sequences: those that initiate antigen-dependent primary activation through the TCR (primary cytoplasmic signaling sequences), and those that act in an antigen-independent manner to provide a secondary or co-stimulatory signal (secondary cytoplasmic signaling sequences). In some aspects, the CAR includes one or both of such classes of cytoplasmic signaling sequences.

In some aspects, the CAR includes a primary cytoplasmic signaling sequence that regulates primary stimulation and/or activation of the TCR complex. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences include those derived from TCR or CD3 zeta, FcR gamma, CD3 gamma, CD3 delta and CD3 epsilon. In some embodiments, the intracellular signaling region in the CAR contain(s) a cytoplasmic signaling domain, portion thereof, or sequence derived from CD3 zeta. In some embodiments the CD3 zeta comprises the sequence of amino acids set forth in SEQ ID NO: 57.

In some embodiments, the intracellular signaling domain comprises a human CD3 zeta stimulatory signaling domain or functional variant thereof, such as an 112 AA cytoplasmic domain of isoform 3 of human CD3ζ (Accession No.: P20963.2) or a CD3 zeta signaling domain as described in U.S. Pat. No. 7,446,190 or U.S. Pat. No. 8,911,993. In some embodiments, the intracellular signaling domain comprises the sequence of amino acids set forth in SEQ ID NO: 57 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 57. In some embodiments, the CD3 zeta is or contains the sequence set forth in SEQ ID NO: 57.

In some embodiments, the CAR includes a signaling domain (e.g., an intracellular or cytoplasmic signaling domain) and/or transmembrane portion of a costimulatory molecule, such as a T cell costimulatory molecule. Exemplary costimulatory molecules include CD28, 4-1BB, OX40, DAP10, CD2, CD40, CD7, CD27, GITR, and ICOS. For example, a costimulatory molecule can be derived from 4-1BB. In some embodiments, the CAR costimulatory domain is derived from immune-stimulatory receptors such as TACI, BAFF-R, or BCMA. In some embodiments, the costimulatory molecule from 4-1BB is encoded by a polynucleotide that has been optionally optimized for codon usage and/or to reduce RNA heterogeneity, e.g., by removing cryptic splice sites. In some embodiments, the costimulatory molecule from 4-1BB comprises the amino acid sequence set forth in SEQ ID NO: 56. In some embodiments, the intracellular domain comprises an intracellular costimulatory signaling domain of 4-1BB or functional variant or portion thereof, such as a 42-amino acid cytoplasmic domain of a human 4-1BB (Accession No. Q07011.1) or functional variant or portion thereof, such as the sequence of amino acids set forth in SEQ ID NO: 56 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 56. In some aspects, the same CAR includes both the stimulatory or activating components (e.g., cytoplasmic signaling sequence) and costimulatory components.

In some aspects, the transmembrane domain contains a transmembrane portion of CD28. The extracellular binding domain and transmembrane can be linked directly or indirectly. In some embodiments, the extracellular binding domain and transmembrane are linked by a spacer, such as any described herein. In some embodiments, the chimeric antigen receptor contains an intracellular domain of a T cell costimulatory molecule, such as between the transmembrane domain and intracellular signaling domain. In some aspects, the T cell costimulatory molecule is 4-1BB.

In some embodiments, the stimulatory or activating components are included within one CAR, whereas the costimulatory component is provided by another CAR recognizing another antigen. In some embodiments, the CARs include activating or stimulatory CARs, and costimulatory CARs, both expressed on the same cell (see WO 2014/055668). In some aspects, the CAR is the stimulatory or activating CAR; in other aspects, it is the costimulatory CAR. In some embodiments, the cells further include inhibitory CARs (iCARs, see Fedorov et al., Sci. Transl. Medicine, 5(215) (December 2013) whereby a stimulatory or an activating signal delivered through the bispecific tandem CAR is diminished or inhibited by binding of the inhibitory CAR to its ligand, e.g., to reduce off-target effects.

In some embodiments, the two receptors induce, respectively, an activating and an inhibitory signal to the cell, such that ligation of one of the receptor to its antigen activates the cell or induces a response, but ligation of the second inhibitory receptor to its antigen induces a signal that suppresses or dampens that response. Examples are combinations of activating CARs and inhibitory CARs. Such a strategy may be used, for example, to reduce the likelihood of off-target effects in the context in which the activating CAR binds an antigen expressed in a disease or condition but which is also expressed on normal cells, and the inhibitory receptor binds to a separate antigen which is expressed on the normal cells but not cells of the disease or condition.

In some aspects, the chimeric receptor is or includes an inhibitory CAR and includes intracellular components that dampen or suppress an immune response, such as an ITAM- and/or co stimulatory-promoted response in the cell. Exemplary of such intracellular signaling components are those found on immune checkpoint molecules, including PD-1, CTLA4, LAG3, BTLA, OX2R, TIM-3, TIGIT, LAIR-1, PGE2 receptors, EP2/4 Adenosine receptors including A2AR. In some aspects, the engineered cell includes an inhibitory CAR including a signaling domain of or derived from such an inhibitory molecule, such that it serves to dampen the response of the cell, for example, that induced by an activating and/or costimulatory CAR.

In certain embodiments, the intracellular signaling region comprises a CD28 transmembrane and signaling domain linked to a CD3 (e.g., CD3-zeta) intracellular domain. In some embodiments, the intracellular signaling domain comprises a chimeric CD28 and 4-1BB (CD137; TNFRSF9) co-stimulatory domains, linked to a CD3 zeta intracellular domain.

In some embodiments, the CAR encompasses one or more, e.g., two or more, costimulatory domains and a stimulatory or an activation domain, e.g., primary activation domain, in the cytoplasmic portion. Exemplary CARs include intracellular components of CD3-zeta and 4-1BB.

### E. Exemplary CARs

In some of or any of the provided embodiments, the bispecific CAR and/or the BAFF-R-binding domain, antibody or antigen binding fragment, specifically binds to BAFF-R, such as BAFF-R on the surface of a cancer cell. In some embodiments binding can be to a human BAFF-R, a mouse BAFF-R protein, or a non-human primate (e.g., cynomolgus monkey) BAFF-R protein. In some embodiments, among provided bispecific CARs and/or BAFF-R-binding domain are those that bind human BAFF-R protein. The observation that an antibody or other binding molecule binds to BAFF-R protein or specifically binds to BAFF-R protein does not necessarily mean that it binds to a BAFF-R protein of every species. For example, in some embodiments, features of binding to BAFF-R protein, such as the ability to specifically bind thereto and/or to compete for binding thereto with a reference antibody, and/or to bind with a particular affinity or compete to a particular degree, in some embodiments, refers to the ability with respect to a human BAFF-R protein and the antibody may not have this feature with respect to a BAFF-R protein of another species, such as mouse. In some embodiments, the antibodies specifically bind to human BAFF-R protein, such as to an epitope or region of human BAFF-R protein.

In one embodiment, the extent of binding of an anti-BAFF-R antibody or antigen-binding domain or CAR to an unrelated, non-BAFF-R protein, such as a non-human BAFF-R protein or other non-BAFF-R protein, is less than at or about 10% of the binding of the antibody or antigen-binding domain or CAR to human BAFF-R protein or human membrane-bound BAFF-R as measured, e.g., by a radioimmunoassay (RIA). In some embodiments, among the antibodies or antigen-binding domains in the provided CARs, are antibodies or antigen-binding domains or CARs in which binding to mouse BAFF-R protein is less than or at or about 10% of the binding of the antibody to human BAFF-R protein. In some embodiments, among the antibodies or antigen-binding domains in the provided CARs, are antibodies in which binding to cynomolgus monkey BAFF-R protein is less than or at or about 10% of the binding of the antibody to human BAFF-R protein. In some embodiments, among the antibodies or antigen-binding domains in the provided CARs, are antibodies in which binding to cynomolgus monkey BAFF-R protein and/or a mouse BAFF-R protein is similar to or about the same as the binding of the antibody to human BAFF-R protein.

In some embodiments, the antibodies, in the provided CARs, are capable of binding BAFF-R protein, such as human BAFF-R protein, with at least a certain affinity, as measured by any of a number of known methods. In some embodiments, the affinity is represented by an equilibrium dissociation constant (K_{D}); in some embodiments, the affinity is represented by EC₅₀.

In some cases, the BAFF-R domain of the bispecific CAR can be blocked or inhibited by the presence of soluble BAFF. In some embodiments, the binding of the BAFF-R domain of the bispecific CAR to BAFF on target cells is reduced in the presence of soluble BAFF. In some embodiments, the binding of the BAFF-R domain of the bispecific CAR to BAFF on target cells in the presence of soluble BAFF is reduced no more than 40% relative to binding in the absence of soluble BAFF. In some embodiments, the binding of the BAFF-R domain of the bispecific CAR to BAFF on target cells in the presence of soluble BAFF is reduced no more than 30% relative to binding in the absence of soluble BAFF. In some embodiments, the binding of the BAFF-R domain of the bispecific CAR to BAFF on target cells in the presence of soluble BAFF is reduced no more than 20% relative to binding in the absence of soluble BAFF. In some embodiments, the binding of the BAFF-R domain of the bispecific CAR to BAFF on target cells in the presence of soluble BAFF is reduced no more than 10% relative to binding in the absence of soluble BAFF. In some embodiments, the binding of the BAFF-R domain of the bispecific CAR is not reduced or blocked or is not substantially reduced or blocked in the presence of soluble BAFF.

In some cases, the CD19 domain of the bispecific CAR can be blocked or inhibited by the presence of FMC63 scFv. In some embodiments, the binding of the CD19 domain of the bispecific CAR to CD19 on target cells is reduced in the presence of FMC63 scFv. In some embodiments, the binding of the CD19 domain of the bispecific CAR to CD19 on target cells in the presence of FMC63 scFv is reduced no more than 50% relative to binding in the absence of FMC63 scFv. In some embodiments, the binding of the CD19 domain of the bispecific CAR to CD19 on target cells in the presence of FMC63 scFv is reduced no more than 40% relative to binding in the absence of FMC63 scFv. In some embodiments, the binding of the CD19 domain of the bispecific CAR to CD19 on target cells in the presence of FMC63 scFv is reduced no more than 30% relative to binding in the absence of FMC63 scFv. In some embodiments, the binding of the CD19 domain of the bispecific CAR to CD19 on target cells in the presence of FMC63 scFv is reduced no more than 20% relative to binding in the absence of FMC63 scFv. In some embodiments, the binding of the CD19 domain of the bispecific CAR to CD19 on target cells in the presence of FMC63 scFv is reduced no more than 10% relative to binding in the absence of FMC63 scFv. In some embodiments, the binding of the CD19 domain of the bispecific CAR is not reduced or blocked or is not substantially reduced or blocked in the presence of FMC63 scFv.

A variety of assays are known for assessing binding affinity and/or determining whether a binding molecule (e.g., an antibody or fragment thereof) specifically binds to a particular ligand (e.g., an antigen). It is within the level of a skilled artisan to determine the binding affinity of a binding molecule, e.g., an antibody, for an antigen, such as by using any of a number of binding assays that are well known in the art. For example, in some embodiments, a BIAcore® instrument can be used to determine the binding kinetics and constants of a complex between two proteins (e.g., an antibody or fragment thereof, and an antigen, such as BAFF-R), using surface plasmon resonance (SPR) analysis (see, e.g., Scatchard et al., Ann. N.Y. Acad. Sci. 51:660, 1949; Wilson, Science 295:2103, 2002; Wolff et al., Cancer Res. 53:2560, 1993; and U.S. Pat. Nos. 5,283,173, 5,468,614, or the equivalent).

In some of or any of the provided embodiments, the bispecific CAR and/or the CD19-binding domain, antibody or antigen binding fragment, specifically binds to CD19, such as CD19 on the surface of a cancer cell. In some embodiments binding can be to a human CD19, a mouse CD19 protein, or a non-human primate (e.g., cynomolgus monkey) CD19 protein. In some embodiments, among provided bispecific CARs and/or CD19-binding domain are those that bind human CD19 protein. The observation that an antibody or other binding molecule binds to CD19 protein or specifically binds to D19 protein does not necessarily mean that it binds to a CD19 protein of every species. For example, in some embodiments, features of binding to CD19 protein, such as the ability to specifically bind thereto and/or to compete for binding thereto with a reference antibody, and/or to bind with a particular affinity or compete to a particular degree, in some embodiments, refers to the ability with respect to a human CD19 protein and the antibody may not have this feature with respect to a CD19 protein of another species, such as mouse. In some embodiments, the antibodies specifically bind to human CD19 protein, such as to an epitope or region of human CD19 protein.

In one embodiment, the extent of binding of an anti-CD19 antibody or antigen-binding domain or CAR to an unrelated, non-CD19 protein, such as a non-human CD19 protein or other non-CD19 protein, is less than at or about 10% of the binding of the antibody or antigen-binding domain or CAR to human CD19 protein or human membrane-bound CD19 as measured, e.g., by a radioimmunoassay (RIA). In some embodiments, among the antibodies or antigen-binding domains in the provided CARs, are antibodies or antigen-binding domains or CARs in which binding to mouse CD19 protein is less than or at or about 10% of the binding of the antibody to human CD19 protein. In some embodiments, among the antibodies or antigen-binding domains in the provided CARs, are antibodies in which binding to cynomolgus monkey CD19 protein is less than or at or about 10% of the binding of the antibody to human CD19 protein. In some embodiments, among the antibodies or antigen-binding domains in the provided CARs, are antibodies in which binding to cynomolgus monkey CD19 protein and/or a mouse CD19 protein is similar to or about the same as the binding of the antibody to human CD19 protein.

In some embodiments, the antibodies, in the provided CARs, are capable of binding CD19 protein, such as human CD19 protein, with at least a certain affinity, as measured by any of a number of known methods. In some embodiments, the affinity is represented by an equilibrium dissociation constant (K_{D}); in some embodiments, the affinity is represented by EC₅₀.

A variety of assays are known for assessing binding affinity and/or determining whether a binding molecule (e.g., an antibody or fragment thereof) specifically binds to a particular ligand (e.g., an antigen, such as CD19). It is within the level of a skilled artisan to determine the binding affinity of a binding molecule, e.g., an antibody, for an antigen, e.g., CD19, such as human CD19 or cynomolgus CD19 or mouse CD19, such as by using any of a number of binding assays that are well known in the art. For example, in some embodiments, a BIAcore® instrument can be used to determine the binding kinetics and constants of a complex between two proteins (e.g., an antibody or fragment thereof, and an antigen, such as CD19), using surface plasmon resonance (SPR) analysis (see, e.g., Scatchard et al., Ann. N.Y. Acad. Sci. 51:660, 1949; Wilson, Science 295:2103, 2002; Wolff et al., Cancer Res. 53:2560, 1993; and U.S. Pat. Nos. 5,283,173, 5,468,614, or the equivalent).

SPR measures changes in the concentration of molecules at a sensor surface as molecules bind to or dissociate from the surface. The change in the SPR signal is directly proportional to the change in mass concentration close to the surface, thereby allowing measurement of binding kinetics between two molecules. The dissociation constant for the complex can be determined by monitoring changes in the refractive index with respect to time as buffer is passed over the chip. Other suitable assays for measuring the binding of one protein to another include, for example, immunoassays such as enzyme linked immunosorbent assays (ELISA) and radioimmunoassays (RIA), or determination of binding by monitoring the change in the spectroscopic or optical properties of the proteins through fluorescence, UV absorption, circular dichroism, or nuclear magnetic resonance (NMR). Other exemplary assays include, but are not limited to, Western blot, ELISA, analytical ultracentrifugation, spectroscopy, flow cytometry, sequencing and other methods for detection of expressed polynucleotides or binding of proteins.

In some embodiments, the binding molecule, e.g., antibody or fragment thereof or antigen-binding domain of a CAR, binds, such as specifically binds, to an antigen, e.g., a BAFF-R protein or an epitope therein, with an affinity or K_{A} (i.e., an equilibrium association constant of a particular binding interaction with units of 1/M; equal to the ratio of the on-rate [kₒₙ or kₐ] to the off-rate [k_{off} or k_{d}] for this association reaction, assuming bimolecular interaction) equal to or greater than 10⁵ M⁻¹. In some embodiments, the antibody or fragment thereof or antigen-binding domain of a CAR exhibits a binding affinity for the peptide epitope with a K_{D} (i.e., an equilibrium dissociation constant of a particular binding interaction with units of M; equal to the ratio of the off-rate [k_{off} or k_{d}] to the on-rate [kₒₙ or kₐ] for this association reaction, assuming bimolecular interaction) of equal to or less than 10⁻⁵ M. For example, the equilibrium dissociation constant K_{D} ranges from 10⁻⁵ M to 10⁻¹³ M, such as 10⁻⁷ M to 10⁻¹¹ M, 10⁻⁸ M to 10⁻¹⁰ M, or 10⁻⁹ M to 10⁻¹⁰ M. The on-rate (association rate constant; kₒₙ or kₐ; units of 1/Ms) and the off-rate (dissociation rate constant; k_{off} or k_{d}; units of l/s) can be determined using any of the assay methods known in the art, for example, surface plasmon resonance (SPR).

In some embodiments, the binding affinity (EC₅₀) and/or the dissociation constant of the antibody (e.g. antigen-binding fragment) or antigen-binding domain of a CAR to BAFF-R protein, such as human BAFF-R protein, is from or from about 0.01 nM to about 500 nM, from or from about 0.01 nM to about 400 nM, from or from about 0.01 nM to about 100 nM, from or from about 0.01 nM to about 50 nM, from or from about 0.01 nM to about 10 nM, from or from about 0.01 nM to about 1 nM, from or from about 0.01 nM to about 0.1 nM, from or from about 0.1 nM to about 500 nM, from or from about 0.1 nM to about 400 nM, from or from about 0.1 nM to about 100 nM, from or from about 0.1 nM to about 50 nM, from or from about 0.1 nM to about 10 nM, from or from about 0.1 nM to about 1 nM, from or from about 0.5 nM to about 200 nM, from or from about 1 nM to about 500 nM, from or from about 1 nM to about 100 nM, from or from about 1 nM to about 50 nM, from or from about 1 nM to about 10 nM, from or from about 2 nM to about 50 nM, from or from about 10 nM to about 500 nM, from or from about 10 nM to about 100 nM, from or from about 10 nM to about 50 nM, from or from about 50 nM to about 500 nM, from or from about 50 nM to about 100 nM or from or from about 100 nM to about 500 nM. In certain embodiments, the binding affinity (EC₅₀) and/or the equilibrium dissociation constant, K_{D}, of the antibody to a BAFF-R protein, such as human BAFF-R protein, is at or less than or about 400 nM, 300 nM, 200 nM, 100 nM, 50 nM, 40 nM, 30 nM, 25 nM, 20 nM, 19 nM, 18 nM, 17 nM, 16 nM, 15 nM, 14 nM, 13 nM, 12 nM, 11 nM, 10 nM, 9 nM, 8 nM, 7 nM, 6 nM, 5 nM, 4 nM, 3 nM, 2 nM, or 1 nM or less. In some embodiments, the antibodies bind to a BAFF-R protein, such as human BAFF-R protein, with a sub-nanomolar binding affinity, for example, with a binding affinity less than about 1 nM, such as less than about 0.9 nM, about 0.8 nM, about 0.7 nM, about 0.6 nM, about 0.5 nM, about 0.4 nM, about 0.3 nM, about 0.2 nM or about 0.1 nM or less.

In some embodiments, the binding affinity may be classified as high affinity or as low affinity. In some cases, the binding molecule (e.g. antibody or fragment thereof) or antigen-binding domain of a CAR that exhibits low to moderate affinity binding exhibits a K_{A} of up to 10⁷ M⁻¹, up to 10⁶ M⁻¹, up to 10⁵ M⁻¹. In some cases, a binding molecule (e.g. antibody or fragment thereof) that exhibits high affinity binding to a particular epitope interacts with such epitope with a K_{A} of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹. In some embodiments, the binding affinity (EC₅₀) and/or the equilibrium dissociation constant, K_{D}, of the binding molecule, e.g., anti-BAFF-R antibody or fragment thereof or antigen-binding domain of a CAR, to a BAFF-R protein, is from or from about 0.01 nM to about 1 μM, 0.1 nM to 1 μM, 1 nM to 1μ, 1 nM to 500 nM, 1 nM to 100 nM, 1 nM to 50 nM, 1 nM to 10 nM, 10 nM to 500 nM, 10 nM to 100 nM, 10 nM to 50 nM, 50 nM to 500 nM, 50 nM to 100 nM or 100 nM to 500 nM. In certain embodiments, the binding affinity (EC₅₀) and/or the dissociation constant of the equilibrium dissociation constant, K_{D}, of the binding molecule, e.g., anti-BAFF-R antibody or fragment thereof or antigen-binding domain of a CAR, to a BAFF-R protein, is at or about or less than at or about 1 μM, 500 nM, 100 nM, 50 nM, 40 nM, 30 nM, 25 nM, 20 nM, 19 nM, 18 nM, 17 nM, 16 nM, 15 nM, 14 nM, 13 nM, 12 nM, 11 nM, 10 nM, 9 nM, 8 nM, 7 nM, 6 nM, 5 nM, 4 nM, 3 nM, 2 nM, or 1 nM or less. The degree of affinity of a particular antibody can be compared with the affinity of a known antibody, such as a reference antibody.

In some embodiments, the binding affinity of a binding molecule, such as an anti-BAFF-R antibody or antigen-binding domain of a CAR, for different antigens, e.g., BAFF-R proteins from different species can be compared to determine the species cross-reactivity. For example, species cross-reactivity can be classified as high cross reactivity or low cross reactivity. In some embodiments, the equilibrium dissociation constant, K_{D}, for different antigens, e.g., BAFF-R proteins from different species such as human, cynomolgus monkey or mouse, can be compared to determine species cross-reactivity. In some embodiments, the species cross-reactivity of an anti-BAFF-R antibody or antigen-binding domain of a CAR can be high, e.g., the anti-BAFF-R antibody binds to human BAFF-R and a species variant BAFF-R to a similar degree, e.g., the ratio of K_{D} for human BAFF-R and K_{D} for the species variant BAFF-R is or is about 1. In some embodiments, the species cross-reactivity of an anti-BAFF-R antibody or antigen-binding domain of a CAR can be low, e.g., the anti-BAFF-R antibody has a high affinity for human BAFF-R but a low affinity for a species variant BAFF-R, or vice versa. For example, the ratio of K_{D} for the species variant BAFF-R and K_{D} for the human BAFF-R is more than 10, 15, 20, 25, 30, 40, 50, 60, 70, 80, 90, 100, 200, 500, 1000, 2000 or more, and the anti-BAFF-R antibody has low species cross-reactivity. The degree of species cross-reactivity can be compared with the species cross-reactivity of a known antibody, such as a reference antibody.

Among the provided bispecific CARs are CARs that exhibit antigen-dependent activity or signaling, i.e. signaling activity that is measurably absent or at background levels in the absence of antigen, e.g. BAFF-R. Thus, in some aspects, provided CARs do not exhibit, or exhibit no more than background or a tolerable or low level of, tonic signaling or antigen-independent activity or signaling in the absence of antigen, e.g. BAFF-R, being present. In some embodiments, the provided bispecific CAR-expressing cells exhibit biological activity or function, including cytotoxic activity, cytokine production, and ability to proliferate.

In some embodiments, the binding molecule, e.g., antibody or fragment thereof or antigen-binding domain of a CAR, binds, such as specifically binds, to an antigen, e.g., a CD19 protein or an epitope therein, with an affinity or K_{A} (i.e., an equilibrium association constant of a particular binding interaction with units of 1/M; equal to the ratio of the on-rate [kₒₙ or kₐ] to the off-rate [k_{off} or k_{d}] for this association reaction, assuming bimolecular interaction) equal to or greater than 10⁵ M⁻¹. In some embodiments, the antibody or fragment thereof or antigen-binding domain of a CAR exhibits a binding affinity for the peptide epitope with a K_{D} (i.e., an equilibrium dissociation constant of a particular binding interaction with units of M; equal to the ratio of the off-rate [k_{off} or k_{d}] to the on-rate [kₒₙ or kₐ] for this association reaction, assuming bimolecular interaction) of equal to or less than 10⁻⁵ M. For example, the equilibrium dissociation constant K_{D} ranges from 10⁻⁵ M to 10⁻¹³ M, such as 10⁻⁷ M to 10⁻¹¹ M, 10⁻⁸ M to 10⁻¹⁰ M, or 10⁻⁹ M to 10⁻¹⁰ M. The on-rate (association rate constant; kₒₙ or kₐ; units of 1/Ms) and the off-rate (dissociation rate constant; k_{off} or k_{d}; units of l/s) can be determined using any of the assay methods known in the art, for example, surface plasmon resonance (SPR).

In some embodiments, the binding affinity (EC₅₀) and/or the dissociation constant of the antibody (e.g. antigen-binding fragment) or antigen-binding domain of a CAR to CD19 protein, such as human CD19 protein, is from or from about 0.01 nM to about 500 nM, from or from about 0.01 nM to about 400 nM, from or from about 0.01 nM to about 100 nM, from or from about 0.01 nM to about 50 nM, from or from about 0.01 nM to about 10 nM, from or from about 0.01 nM to about 1 nM, from or from about 0.01 nM to about 0.1 nM, is from or from about 0.1 nM to about 500 nM, from or from about 0.1 nM to about 400 nM, from or from about 0.1 nM to about 100 nM, from or from about 0.1 nM to about 50 nM, from or from about 0.1 nM to about 10 nM, from or from about 0.1 nM to about 1 nM, from or from about 0.5 nM to about 200 nM, from or from about 1 nM to about 500 nM, from or from about 1 nM to about 100 nM, from or from about 1 nM to about 50 nM, from or from about 1 nM to about 10 nM, from or from about 2 nM to about 50 nM, from or from about 10 nM to about 500 nM, from or from about 10 nM to about 100 nM, from or from about 10 nM to about 50 nM, from or from about 50 nM to about 500 nM, from or from about 50 nM to about 100 nM or from or from about 100 nM to about 500 nM. In certain embodiments, the binding affinity (EC₅₀) and/or the equilibrium dissociation constant, K_{D}, of the antibody to a CD19 protein, such as human CD19 protein, is at or less than or about 400 nM, 300 nM, 200 nM, 100 nM, 50 nM, 40 nM, 30 nM, 25 nM, 20 nM, 19 nM, 18 nM, 17 nM, 16 nM, 15 nM, 14 nM, 13 nM, 12 nM, 11 nM, 10 nM, 9 nM, 8 nM, 7 nM, 6 nM, 5 nM, 4 nM, 3 nM, 2 nM, or 1 nM or less. In some embodiments, the antibodies bind to a CD19 protein, such as human CD19 protein, with a sub-nanomolar binding affinity, for example, with a binding affinity less than about 1 nM, such as less than about 0.9 nM, about 0.8 nM, about 0.7 nM, about 0.6 nM, about 0.5 nM, about 0.4 nM, about 0.3 nM, about 0.2 nM or about 0.1 nM or less.

In some embodiments, the binding affinity may be classified as high affinity or as low affinity. In some cases, the binding molecule (e.g. antibody or fragment thereof) or antigen-binding domain of a CAR that exhibits low to moderate affinity binding exhibits a K_{A} of up to 10⁷ M⁻¹, up to 10⁶ M⁻¹, up to 10⁵ M⁻¹. In some cases, a binding molecule (e.g. antibody or fragment thereof) that exhibits high affinity binding to a particular epitope interacts with such epitope with a K_{A} of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹. In some embodiments, the binding affinity (EC₅₀) and/or the equilibrium dissociation constant, K_{D}, of the binding molecule, e.g., anti-CD19 antibody or fragment thereof or antigen-binding domain of a CAR, to a CD19 protein, is from or from about 0.01 nM to about 1 μM, 0.1 nM to 1 μM, 1 nM to 1 μM, 1 nM to 500 nM, 1 nM to 100 nM, 1 nM to 50 nM, 1 nM to 10 nM, 10 nM to 500 nM, 10 nM to 100 nM, 10 nM to 50 nM, 50 nM to 500 nM, 50 nM to 100 nM or 100 nM to 500 nM. In certain embodiments, the binding affinity (EC₅₀) and/or the dissociation constant of the equilibrium dissociation constant, K_{D}, of the binding molecule, e.g., anti-CD19 antibody or fragment thereof or antigen-binding domain of a CAR, to a CD19 protein, is at or about or less than at or about 1 μM, 500 nM, 100 nM, 50 nM, 40 nM, 30 nM, 25 nM, 20 nM, 19 nM, 18 nM, 17 nM, 16 nM, 15 nM, 14 nM, 13 nM, 12 nM, 11 nM, 10 nM, 9 nM, 8 nM, 7 nM, 6 nM, 5 nM, 4 nM, 3 nM, 2 nM, or 1 nM or less. The degree of affinity of a particular antibody can be compared with the affinity of a known antibody, such as a reference antibody.

In some embodiments, the binding affinity of a binding molecule, such as an anti-CD19 antibody or antigen-binding domain of a CAR, for different antigens, e.g., CD19 proteins from different species can be compared to determine the species cross-reactivity. For example, species cross-reactivity can be classified as high cross reactivity or low cross reactivity. In some embodiments, the equilibrium dissociation constant, K_{D}, for different antigens, e.g., CD19 proteins from different species such as human, cynomolgus monkey or mouse, can be compared to determine species cross-reactivity. In some embodiments, the species cross-reactivity of an anti-CD19 antibody or antigen-binding domain of a CAR can be high, e.g., the anti-CD19 antibody binds to human CD19 and a species variant CD19 to a similar degree, e.g., the ratio of K_{D} for human CD19 and K_{D} for the species variant CD19 is or is about 1. In some embodiments, the species cross-reactivity of an anti-CD19 antibody or antigen-binding domain of a CAR can be low, e.g., the anti-CD19 antibody has a high affinity for human CD19 but a low affinity for a species variant CD19, or vice versa. For example, the ratio of K_{D} for the species variant CD19 and K_{D} for the human CD19 is more than 10, 15, 20, 25, 30, 40, 50, 60, 70, 80, 90, 100, 200, 500, 1000, 2000 or more, and the anti-CD19 antibody has low species cross-reactivity. The degree of species cross-reactivity can be compared with the species cross-reactivity of a known antibody, such as a reference antibody.

Among the provided bispecific CARs are CARs that exhibit antigen-dependent activity or signaling, i.e. signaling activity that is measurably absent or at background levels in the absence of antigen, e.g. CD19. Thus, in some aspects, provided CARs do not exhibit, or exhibit no more than background or a tolerable or low level of, tonic signaling or antigen-independent activity or signaling in the absence of antigen, e.g. CD19, being present. In some embodiments, the provided bispecific CAR-expressing cells exhibit biological activity or function, including cytotoxic activity, cytokine production, and ability to proliferate.

In some embodiments, biological activity or functional activity of a chimeric receptor, such as cytotoxic activity, can be measured using any of a number of known methods. The activity can be assessed or determined either in vitro or in vivo. In some embodiments, activity can be assessed once the cells are administered to the subject (e.g., human). Parameters to assess include specific binding of an engineered or natural T cell or other immune cell to antigen, e.g., in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells also can be measured by assaying expression and/or secretion of certain cytokines, such as interlekukin-2 (IL-2), interferon-gamma (IFNγ), interleukin-4 (IL-4), TNF-alpha (TNFα), interleukin-6 (IL-6), interleukin-10 (IL-10), interleukin-12 (IL-12), granulocyte-macrophage colony-stimulating factor (GM-CSF), CD107a, and/or TGF-beta (TGFβ). Assays to measure cytokines are well known in the art, and include but are not limited to, ELISA, intracellular cytokine staining, cytometric bead array, RT-PCR, ELISPOT, flow cytometry and bio-assays in which cells responsive to the relevant cytokine are tested for responsiveness (e.g. proliferation) in the presence of a test sample. In some aspects, the biological activity is measured by assessing clinical outcome, such as reduction in tumor burden or load.

In some aspects, a reporter cell line can be employed to monitor antigen-independent activity and/or tonic signaling through bispecific CAR-expressing cells. In some embodiments, a T cell line, such as a Jurkat cell line (which is CD19-negative/BAFF-R-negative), contains a reporter molecule, such as a fluorescent protein or other detectable molecule, such as a red fluorescent protein, expressed under the control of the endogenous Nur77 transcriptional regulatory elements. In some embodiments, the Nur77 reporter expression is cell intrinsic and dependent upon signaling through a recombinant reporter containing a primary activation signal in a T cell, a signaling domain of a T cell receptor (TCR) component, and/or a signaling domain comprising an immunoreceptor tyrosine-based activation motif (ITAM), such as a CD3ζ chain. Nur77 expression is generally not affected by other signaling pathways such as cytokine signaling or toll-like receptor (TLR) signaling, which may act in a cell extrinsic manner and may not depend on signaling through the recombinant receptor. Thus, only cells that express the exogenous bispecific tandem CAR containing the appropriate signaling regions is capable of expressing Nur77 upon stimulation (e.g., binding of the specific antigen). In some cases, Nur77 expression also can show a dose-dependent response to the amount of stimulation (e.g., antigen).

In some embodiments, the provided bispecific CARs exhibit improved expression on the surface of cells, such as compared to an alternative CAR that has an identical amino acid sequence but that is encoded by non-splice site eliminated and/or a non-codon-optimized nucleotide sequence. In some embodiments, the expression of the recombinant receptor on the surface of the cell can be assessed. Approaches for determining expression of the recombinant receptor on the surface of the cell may include use of chimeric antigen receptor (CAR)-specific antibodies (e.g., Brentjens et al., Sci. Transl. Med. 2013 March; 5(177): 177ra38), Protein L (Zheng et al., J. Transl. Med. 2012 February; 10:29), epitope tags, and monoclonal antibodies that specifically bind to a CAR polypeptide (see international patent application Pub. No. WO2014190273). In some embodiments, the expression of the recombinant receptor on the surface of the cell, e.g., primary T cell, can be assessed, for example, by flow cytometry, using binding molecules that can bind to the recombinant receptor or a portion thereof that can be detected. In some embodiments, the binding molecules used for detecting expression of the recombinant receptor is or comprises an anti-idiotypic antibody, e.g., an anti-idiotypic agonist antibody specific for a binding domain, e.g., scFv, or a portion thereof. In some embodiments, the binding molecule is or comprises an isolated or purified antigen, e.g., recombinantly expressed antigen.

In some embodiments, the CAR comprises, in order from N- to C-terminus: the V_{L} region of the CD19-binding domain set forth in SEQ ID NO: 42, the linker set forth in SEQ ID NO: 60, the V_{H} region of the BAFF-R-binding domain set forth in SEQ ID NO: 1, the linker set forth in SEQ ID NO: 58, the V_{L} region of the BAFF-R-binding domain set forth in SEQ ID NO: 2, the linker set forth in SEQ ID NO: 60, and the V_{H} region of the CD19-binding domain set forth in SEQ ID NO: 41.

In some embodiments, the CAR comprises, in order from N- to C-terminus: the V_{L} region of the CD19-binding domain set forth in SEQ ID NO: 42, the linker set forth in SEQ ID NO: 60, the V_{H} region of the BAFF-R-binding domain set forth in SEQ ID NO: 3, the linker set forth in SEQ ID NO: 58, the V_{L} region of the BAFF-R-binding domain set forth in SEQ ID NO: 4, the linker set forth in SEQ ID NO: 60, and the V_{H} region of the CD19-binding domain set forth in SEQ ID NO: 41.

In some embodiments, the CAR comprises, in order from N- to C-terminus: the V_{L} region of the BAFF-R-binding domain set forth in SEQ ID NO: 4, the linker set forth in SEQ ID NO: 61, the V_{L} region of the CD19-binding domain set forth in SEQ ID NO: 42, the linker set forth in SEQ ID NO: 59, the V_{H} region of the CD19-binding domain set forth in SEQ ID NO: 41, the linker set forth in SEQ ID NO: 61, and the V_{H} region of the BAFF-R-binding domain set forth in SEQ ID NO: 3.

In some embodiments, the CAR comprises, in order from N- to C-terminus: the V_{H} region of the BAFF-R-binding domain set forth in SEQ ID NO: 3, the linker set forth in SEQ ID NO: 60, the V_{L} region of the CD19-binding domain set forth in SEQ ID NO: 42, the linker set forth in SEQ ID NO: 59, the V_{H} region of the CD19-binding domain set forth in SEQ ID NO: 41, the linker set forth in SEQ ID NO: 60, and the V_{L} region of the BAFF-R-binding domain set forth in SEQ ID NO: 4.

In some embodiments, the CAR comprises, in order from N- to C-terminus: the V_{L} region of the BAFF-R-binding domain set forth in SEQ ID NO: 6, the linker set forth in SEQ ID NO: 61, the V_{L} region of the CD19-binding domain set forth in SEQ ID NO: 42, the linker set forth in SEQ ID NO: 59, the V_{H} region of the CD19-binding domain set forth in SEQ ID NO: 41, the linker set forth in SEQ ID NO: 61, and the V_{H} region of the BAFF-R-binding domain set forth in SEQ ID NO: 5.

In some embodiments, the CAR comprises, in order from N- to C-terminus: the V_{L} region of the CD19-binding domain set forth in SEQ ID NO: 42, the linker set forth in SEQ ID NO: 60, the V_{H} region of the BAFF-R-binding domain set forth in SEQ ID NO: 9, the linker set forth in SEQ ID NO: 58, the V_{L} region of the BAFF-R-binding domain set forth in SEQ ID NO: 10, the linker set forth in SEQ ID NO: 60, and the V_{H} region of the CD19-binding domain set forth in SEQ ID NO: 41.

In some embodiments, the CAR comprises, in order from N- to C-terminus: the V_{L} region of the CD19-binding domain set forth in SEQ ID NO: 42, the linker set forth in SEQ ID NO: 59, the V_{H} region of the CD19-binding domain set forth in SEQ ID NO: 41, the linker set forth in SEQ ID NO: 60, the V_{H} region of the BAFF-R-binding domain set forth in SEQ ID NO: 3, the linker set forth in SEQ ID NO: 58, and the V_{L} region of the BAFF-R-binding domain set forth in SEQ ID NO: 4.

In some embodiments, the CAR comprises, in order from N- to C-terminus: the V_{L} region of the CD19-binding domain set forth in SEQ ID NO: 42, the linker set forth in SEQ ID NO: 59, the V_{H} region of the CD19-binding domain set forth in SEQ ID NO: 41, the linker set forth in SEQ ID NO: 60, the V_{L} region of the BAFF-R-binding domain set forth in SEQ ID NO: 4, the linker set forth in SEQ ID NO: 58, and the V_{H} region of the BAFF-R-binding domain set forth in SEQ ID NO: 3.

In some embodiments, the CAR comprises, in order from N- to C-terminus: the V_{L} region of the CD19-binding domain set forth in SEQ ID NO: 42, the linker set forth in SEQ ID NO: 59, the V_{H} region of the CD19-binding domain set forth in SEQ ID NO: 41, the linker set forth in SEQ ID NO: 60, the V_{H} region of the BAFF-R-binding domain set forth in SEQ ID NO: 7, the linker set forth in SEQ ID NO: 58, and the V_{L} region of the BAFF-R-binding domain set forth in SEQ ID NO: 8.

In some embodiments, the CAR comprises, in order from N- to C-terminus: the V_{L} region of the CD19-binding domain set forth in SEQ ID NO: 42, the linker set forth in SEQ ID NO: 59, the V_{H} region of the CD19-binding domain set forth in SEQ ID NO: 41, the linker set forth in SEQ ID NO: 62, the V_{L} region of the BAFF-R-binding domain set forth in SEQ ID NO: 4, the linker set forth in SEQ ID NO: 58, and the V_{H} region of the BAFF-R-binding domain set forth in SEQ ID NO: 3.

In some of any embodiments, the bispecific CAR comprises the amino acid sequence set forth in SEQ ID NO: 94, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 94. In some of any embodiments, the bispecific CAR comprises the amino acid sequence set forth in SEQ ID NO: 95, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 95. In some of any embodiments, the bispecific CAR comprises the amino acid sequence set forth in SEQ ID NO: 96, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 96.

In some of any embodiments, the bispecific CAR comprises the amino acid sequence set forth in SEQ ID NO: 97, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 97. In some of any embodiments, the bispecific CAR comprises the amino acid sequence set forth in SEQ ID NO: 98, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 98. In some of any embodiments, the bispecific CAR comprises the amino acid sequence set forth in SEQ ID NO: 99, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 99.

In some of any embodiments, the bispecific CAR comprises the amino acid sequence set forth in SEQ ID NO: 100, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 100. In some of any embodiments, the bispecific CAR comprises the amino acid sequence set forth in SEQ ID NO: 101, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 101.

In some of any embodiments, the bispecific CAR comprises the amino acid sequence set forth in SEQ ID NO: 102, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 102. In some of any embodiments, the bispecific CAR comprises the amino acid sequence set forth in SEQ ID NO: 103, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO:103.

In some embodiments, the CAR comprises the amino acid sequence set forth in any one of SEQ ID NOs: 94-103. In some embodiments, the CAR comprises the amino acid sequence set forth in SEQ ID NO: 94. In some embodiments, the CAR comprises the amino acid sequence set forth in SEQ ID NO: 95. In some embodiments, the CAR comprises the amino acid sequence set forth in SEQ ID NO: 96. In some embodiments, the CAR comprises the amino acid sequence set forth in SEQ ID NO: 97. In some embodiments, the CAR comprises the amino acid sequence set forth in SEQ ID NO: 98. In some embodiments, the CAR comprises the amino acid sequence set forth in SEQ ID NO: 99. In some embodiments, the CAR comprises the amino acid sequence set forth in SEQ ID NO: 100. In some embodiments, the CAR comprises the amino acid sequence set forth in SEQ ID NO: 101. In some embodiments, the CAR comprises the amino acid sequence set forth in SEQ ID NO: 102. In some embodiments, the CAR comprises the amino acid sequence set forth in SEQ ID NO: 103.

In some embodiments, the CAR comprises the amino acid sequence of SEQ ID NO: 130 or by an amino acid sequence that exhibits at least 85%, 86%, 87%, 88%, 89%, 90%, 91%, 92%, 93%, 94%, 95%, 96%, 97%, 98%, or 99% sequence identity to the sequence set forth in SEQ ID NO: 130.

In some embodiments, the CAR is encoded by the nucleic acid sequence set forth in SEQ ID NO: 129 or by a nucleic acid sequence that exhibits at least 85%, 86%, 87%, 88%, 89%, 90%, 91%, 92%, 93%, 94%, 95%, 96%, 97%, 98%, or 99% sequence identity to the sequence set forth in SEQ ID NO: 129.

### F. Polynucleotides Encoding the Recombinant Receptor

Also provided are polynucleotides encoding the chimeric antigen receptor and/or portions, e.g., chains, thereof. Among the provided polynucleotides are those encoding the bispecific chimeric antigen receptors (e.g., antigen-binding fragment) binding BAFF-R and CD19 described herein. The polynucleotides may include those encompassing natural and/or non-naturally occurring nucleotides and bases, e.g., including those with backbone modifications. The terms “nucleic acid molecule”, “nucleic acid”, “sequence of nucleotides”, and “polynucleotide” may be used interchangeably, and refer to a polymer of nucleotides. Such polymers of nucleotides may contain natural and/or non-natural nucleotides, and include, but are not limited to, DNA, RNA, and PNA. “Nucleic acid sequence” refers to the linear sequence of nucleotides that comprise the nucleic acid molecule or polynucleotide.

In some embodiments, the extracellular binding domains comprises, in any order: one of the V_{H} region and the V_{L} region of the BAFF-R-binding domain; the other of the V_{H} region and the V_{L} region of the BAFF-R-binding domain; one of the V_{H} region and the V_{L} region of the CD19-binding domain; and the other of the V_{H} region and the V_{L} region of the CD19-binding domain. In some cases, the polynucleotide encoding the BAFF-R-binding domain and/or the CD19-binding domain contains a signal sequence that encodes a signal peptide, in some cases encoded upstream of the nucleic acid sequences encoding the BAFF-R-binding domain and/or the CD19-binding domain, or joined at the 5′ terminus of the nucleic acid sequences encoding the BAFF-R-binding domain and/or the CD19-binding domain. In some cases, the polynucleotide containing nucleic acid sequences encoding the BAFF-R-binding domain and/or the CD19-binding domain contain a signal sequence that encodes a signal peptide.

In some aspects, the signal sequence may encode a signal peptide derived from a native polypeptide. In other aspects, the signal sequence may encode a heterologous or non-native signal peptide. In some aspects, non-limiting exemplary signal peptide include a signal peptide of a CD33 signal peptide set forth in SEQ ID NO: 104. In some cases, the polynucleotide encoding the BAFF-R-binding domain can contain nucleic acid sequence encoding additional molecules, such as a surrogate marker or other markers, or can contain additional components, such as promoters, regulatory elements and/or multicistronic elements. In some embodiments, the nucleic acid sequence encoding the bispecific tandem CARs can be operably linked to any of the additional components.

In some embodiments, among CARs provided herein are those encoded by polynucleotides that are optimized, or contain certain features designed for optimization, such as for codon usage, to reduce RNA heterogeneity and/or to modify, e.g., increase or render more consistent among cell product lots, expression, such as surface expression, of the encoded receptor. In some embodiments, polynucleotides, encoding BAFF-R-binding domains or CD19-binding domains, are modified as compared to a reference polynucleotide, such as to remove cryptic or hidden splice sites, to reduce RNA heterogeneity. In some embodiments, polynucleotides, encoding BAFF-R-binding and CD19-binding domains, are codon optimized, such as for expression in a mammalian, e.g., human, cell, such as in a human T cell. In some aspects, the modified polynucleotides result in in improved, e.g., increased or more uniform or more consistent level of, expression, e.g., surface expression, when expressed in a cell. Such polynucleotides can be utilized in constructs for generation of engineered cells that express the encoded BAFF-R-binding and CD19-binding domains. Thus, also provided are cells expressing the recombinant receptors encoded by the polynucleotides provided herein and uses thereof in adoptive cell therapy, such as treatment of diseases and disorders associated with BAFF-R and/or CD19 expression.

Also provided are cells, such as T cells, engineered to express a polynucleotide encoding a provided polynucleotide, including polynucleotides encoding a BAFF-R-binding domain and a CD19-binding domain, and compositions containing such cells. In some embodiments, the polynucleotide constructs are codon optimized for expression in a human cell. In some embodiments, one or more splice donor and/or acceptor sites in a polynucleotide construct is modified to reduce heterogeneity of the RNA transcribed from the construct, such as mRNA, following expression in a cell.

### 1. Codon Optimization

In some embodiments the polynucleotides are modified by optimization of the codons for expression in humans. In some aspects, codon optimization can be considered before and/or after the steps for splice site identification and/or splice site elimination, and/or at each of the iterative steps for reducing RNA heterogeneity. Codon optimization generally involves balancing the percentages of codons selected with the abundance, e.g., published abundance, of human transfer RNAs, for example, so that none is overloaded or limiting. In some cases, such balancing is necessary or useful because most amino acids are encoded by more than one codon, and codon usage generally varies from organism to organism. Differences in codon usage between transfected or transduced genes or nucleic acids and host cells can have effects on protein expression from the nucleic acid molecule. Table 2 below sets forth an exemplary human codon usage frequency table. In some embodiments, to generate codon-optimized nucleic acid sequences, codons are chosen to select for those codons that are in balance with human usage frequency. The redundancy of the codons for amino acids is such that different codons code for one amino acid, such as depicted in Table 2. In selecting a codon for replacement, it is desired that the resulting mutation is a silent mutation such that the codon change does not affect the amino acid sequence. Generally, the last nucleotide of the codon (e.g., at the third position) can remain unchanged without affecting the amino acid sequence.

TABLE 2
Human Codon Usage Frequency

| Human | amino | freq./ | | Human | amino | freq./ | |
|---|---|---|---|---|---|---|---|
| codon | acid | 1000 | number | codon | acid | 1000 | number |
| TTT | F | 17.6 | 714298 | TCT | S | 15.2 | 618711 |
| TTC | F | 20.3 | 824692 | TCC | S | 17.7 | 718892 |
| TTA | L | 7.7 | 311881 | TCA | S | 12.2 | 496448 |
| TTG | L | 12.9 | 525688 | TCG | S | 4.4 | 179419 |
| CTT | L | 13.2 | 536515 | CCT | P | 17.5 | 713233 |
| CTC | L | 19.6 | 796638 | CCC | P | 19.8 | 804620 |
| CTA | L | 7.2 | 290751 | CCA | P | 16.9 | 688038 |
| CTG | L | 39.6 | 1611801 | CCG | P | 6.9 | 281570 |
| ATT | I | 16 | 650473 | ACT | T | 13.1 | 533609 |
| ATC | I | 20.8 | 846466 | ACC | T | 18.9 | 768147 |
| ATA | I | 7.5 | 304565 | ACA | T | 15.1 | 614523 |
| ATG | M | 22 | 896005 | ACG | T | 6.1 | 246105 |
| GTT | V | 11 | 448607 | GCT | A | 18.4 | 750096 |
| GTC | V | 14.5 | 588138 | GCC | A | 27.7 | 1127679 |
| GTA | V | 7.1 | 287712 | GCA | A | 15.8 | 643471 |
| GTG | V | 28.1 | 1143534 | GCG | A | 7.4 | 299495 |
| TAT | Y | 12.2 | 495699 | TGT | C | 10.6 | 430311 |
| TAC | Y | 15.3 | 622407 | TGC | C | 12.6 | 513028 |
| TAA | * | 1 | 40285 | TGA | * | 1.6 | 63237 |
| TAG | * | 0.8 | 32109 | TGG | W | 13.2 | 535595 |
| CAT | H | 10.9 | 441711 | CGT | R | 4.5 | 184609 |
| CAC | H | 15.1 | 613713 | CGC | R | 10.4 | 423516 |
| CAA | Q | 12.3 | 501911 | CGA | R | 6.2 | 250760 |
| CAG | Q | 34.2 | 1391973 | CGG | R | 11.4 | 464485 |
| AAT | N | 17 | 689701 | AGT | S | 12.1 | 493429 |
| AAC | N | 19.1 | 776603 | AGC | S | 19.5 | 791383 |
| AAA | K | 24.4 | 993621 | AGA | R | 12.2 | 494682 |
| AAG | K | 31.9 | 1295568 | AGG | R | 12 | 486463 |
| GAT | D | 21.8 | 885429 | GGT | G | 10.8 | 437126 |
| GAC | D | 25.1 | 1020595 | GGC | G | 22.2 | 903565 |
| GAA | E | 29 | 1177632 | GGA | G | 16.5 | 669873 |
| GAG | E | 39.6 | 1609975 | GGG | G | 16.5 | 669768 |

For example, the codons TCT, TCC, TCA, TCG, AGT and AGC all code for Serine (note that T in the DNA equivalent to the U in RNA). From a human codon usage frequency, such as set forth in Table 2 above, the corresponding usage frequencies for these codons are 15.2, 17.7, 12.2, 4.4, 12.1, and 19.5, respectively. Since TCG corresponds to 4.4%, if this codon were commonly used in a gene synthesis, the tRNA for this codon would be limiting. In codon optimization, the goal is to balance the usage of each codon with the normal frequency of usage in the species of animal in which the transgene is intended to be expressed.

### 2. Splice Sites

Provided herein are polynucleotides in which one or more potential splice donor and/or splice acceptor sites have been identified and the nucleic acid sequence at or near the one or more of the identified splice donor sites has been modified. In some embodiments, the resulting modified nucleic acid sequence(s) is/are then synthesized and used to transduce cells to test for splicing as indicated by RNA heterogeneity.

Also provided here are polynucleotides, such as those encoding any of the antibodies, receptors (such as antigen receptors such as chimeric antigen receptors) and/or BAFF-R-specific and/or CD19-specific binding domains provided herein, that are or have been modified to reduce heterogeneity or contain one or more nucleic acid sequences observed herein (such as by the optimization methods) to result in improved features of the polypeptides, such as the CARs, as compared to those containing distinct, reference, sequences or that have not been modified. Among such features include improvements in RNA heterogeneity, such as that resulting from the presence of one or more splice sites, such as one or more cryptic splice sites, and/or improved expression and/or surface expression of the encoded protein, such as increased levels, uniformity, or consistency of expression among cells or different therapeutic cell compositions engineered to express the polypeptides.

Splice sites may be identified in polynucleotide sequences by harvesting RNA from the expressing cells, amplifying by reverse transcriptase polymerase chain reaction (RT-PCR) and resolving by agarose gel electrophoresis to determine the heterogeneity of the RNA, compared to the starting sequence. In some cases, improved sequences can be resubmitted to the gene synthesis vendor for further codon optimization and splice site removal, followed by further cryptic splice site evaluation, modification, synthesis and testing, until the RNA on the agarose gel exhibits minimal RNA heterogeneity.

Also provided are polynucleotides that have been modified to eliminate splice sites, such as cryptic splice sites. Genomic nucleic acid sequences generally, in nature, in a mammalian cell, undergo processing co-transcriptionally or immediately following transcription, wherein a nascent precursor messenger ribonucleic acid (pre-mRNA), transcribed from a genomic deoxyribonucleic acid (DNA) sequence, is in some cases edited by way of splicing, to remove introns, followed by ligation of the exons in eukaryotic cells. Consensus sequences for splice sites are known, but in some aspects, specific nucleotide information defining a splice site may be complex and may not be readily apparent based on available methods. Cryptic splice sites are splice sites that are not predicted based on the standard consensus sequences and are variably activated. Hence, variable splicing of pre-mRNA at cryptic splice sites leads to heterogeneity in the transcribed mRNA products upon expression in eukaryotic cells.

Polynucleotides generated for the expression of transgenes are typically constructed from nucleic acid sequences, such as complementary DNA (cDNA), or portions thereof, that do not contain introns. Thus, splicing of such sequences is not expected to occur. However, the presence of cryptic splice sites within the cDNA sequence can lead to unintended or undesired splicing reactions and heterogeneity in the transcribed mRNA. Such heterogeneity results in translation of unintended protein products, such as truncated protein products with variable amino acid sequences that exhibit modified expression and/or activity.

In some embodiments, eliminating splice sites, such as cryptic splice sites, can improve or optimize expression of a transgene product, such as a polypeptide translated from the transgene, such as a bispecific CAR polypeptide. Splicing at cryptic splice sites of an encoded transgene, such as an encoded CAR comprising a BAFF-R-binding domain and a CD19-binding domain, can lead to reduced protein expression, e.g., expression on cell surfaces, and/or reduced function, e.g., reduced intracellular signaling. Provided herein are polynucleotides, encoding bispecific CAR proteins that have been optimized to reduce or eliminate cryptic splice sites. Also provided herein are polynucleotides encoding bispecific CAR proteins that have been optimized for codon expression and/or in which one or more sequence, such as one identified by the methods or observations herein regarding splice sites, is present, and/or in which an identified splice site, such as any of the identified splice sites herein, is not present. Among the provided polynucleotides are those exhibiting below a certain degree of RNA heterogeneity or splice forms when expressed under certain conditions and/or introduced into a specified cell type, such as a human T cell, such as a primary human T cell, and cells and compositions and articles of manufacture containing such polypeptides and/or exhibiting such properties. In some embodiments, the RNA heterogeneity of transcribed RNA is reduced by greater than or greater than about 10%, 15%, 20%, 25%, 30%, 40%, 50% or more compared to a polynucleotide that has not been modified to remove cryptic splice sites and/or by codon optimization. In some embodiments, the provided polynucleotides encoding a bispecific CAR exhibit RNA homogeneity of transcribed RNA that is at least 70%, 75%, 80%, 85%, 90%, or 95% or greater.

RNA heterogeneity can be determined by any of a number of methods provided herein or described or known. In some embodiments, RNA heterogeneity of a transcribed nucleic acid is determined by amplifying the transcribed nucleic acid, such as by reverse transcriptase polymerase chain reaction (RT-PCR) followed by detecting one or more differences, such as differences in size, in the one or more amplified products. In some embodiments, the RNA heterogeneity is determined based on the number of differently sized amplified products, or the proportion of various differently sized amplified products. In some embodiments, RNA, such as total RNA or cytoplasmic polyadenylated RNA, is harvested from cells, expressing the transgene to be optimized, and amplified by reverse transcriptase polymerase chain reaction (RT-PCR) using a primer specific to the 5′ untranslated region (5′ UTR), in some cases corresponding to a portion of the promoter sequence in the expression vector, located upstream of the transgene in the transcribed RNA, and a primer specific to the 3′ untranslated region (3′ UTR), located downstream of the expressed transgene in the transcribed RNA sequence or a primer specific to a sequence within the transgene. In particular embodiments, at least one primer complementary to a sequence in the 5′ untranslated region (UTR) and at least one primer complementary to a sequence in the 3′ untranslated region (UTR) are employed to amplify the transgene. One can resolve RNA, such as messenger RNA, and analyze the heterogeneity thereof by several methods. Non-limiting, exemplary methods include agarose gel electrophoresis, chip-based capillary electrophoresis, analytical centrifugation, field flow fractionation, and chromatography, such as size exclusion chromatography or liquid chromatography.

In some aspects, the presence of potential cryptic splice sites (splice donor and/or acceptor sites that are present in a transcript, such as a transgene transcript, can result in RNA heterogeneity of the transcript following expression in a cell. In some embodiments, the one or more potential splice sites that can be present in the transgene transcript, that are not desired and/or that may be created in a transgene transcript from various underlying sequences are identified, following codon optimization of a transcript and/or by mutation or mistake or error in transcription. In some aspects of the provided embodiments, the splice donor sites and splice acceptor sites are identified independently. In some embodiments, the splice acceptor and/or donor site(s) is/are canonical, non-canonical, and/or cryptic splice acceptor and/or donor site(s).

In some embodiments, one or more potential splice site (e.g., canonical, non-canonical, and/or cryptic splice acceptor and/or donor site(s) or branch sites) in a polynucleotide, such as a polynucleotide encoding a transgene, such as a recombinant receptor, that may exhibit RNA heterogeneity, are identified and/or modified. Also provided are polypeptides having reduced numbers of such splice sites as compared to such reference polynucleotides.

In some aspects, identification of the one or more splice sites in a nucleic acid sequence is an iterative process. In some embodiments, splice sites can be identified using a splice site and/or codon optimization prediction tool, such as by submitting the starting or reference sequence encoding the transgene, such as a bispecific CAR, or a BAFF-R- or CD19-binding domain comprised therein, to a database, a gene synthesis vendor or other source able to computationally or algorithmically compare the starting or reference sequence to identify or predict splice sites and/or for codon optimization and/or splice site removal. In some embodiments, after modifying the sequence for codon optimization and/or splice site removal, one or more further assessment of a sequence, such as a revised or modified nucleic acid sequence, is carried out to further evaluate for splice site removal, such as cryptic splice sites, using one or more other or additional splice site prediction tool(s).

In some aspects, RNA heterogeneity can be a result of the activity of the spliceosome present in a eukaryotic cell. In some aspects, splicing is typically carried out in a series of reactions catalyzed by the spliceosome. Consensus sequences for splice sites are known, but in some aspects, specific nucleotide information defining a splice site may be complex and may not be readily apparent based on available methods. Cryptic splice sites are splice sites that are not predicted based on the standard consensus sequences and are variably activated. Hence, variable splicing of pre-mRNA at cryptic splice sites leads to heterogeneity in the transcribed mRNA products following expression in eukaryotic cells. In some cases, within spliceosomal introns, a donor site (usually at the 5′ end of the intron), a branch site (near the 3′ end of the intron) and an acceptor site (3′ end of the intron) are required for a splicing event. The splice donor site can include a GU sequence at the 5′ end of the intron, with a large less highly conserved region. The splice acceptor site at the 3′ end of the intron can terminate with an AG sequence.

In some embodiments, splice sites, including potential cryptic splice sites can be identified by comparing sequences to known splice site sequences, such as those in a sequence database. In some embodiments, splice sites can be identified by computationally by submitting nucleotide sequences for analysis by splice site prediction tools, such as Human Splice Finder (Desmet et al., Nucl. Acids Res. 37(9): e67 (2009)), a neural network splice site prediction tool, NNSplice (Reese et al., J. Comput. Biol., 4(4): 311 (1997)), GeneSplicer (Pertea et al., Nucleic Acids Res. 2001 29(5): 1185-1190) or NetUTR (Eden and Brunak, Nucleic Acids Res. 32(3): 1131 (2004)), which identify potential splice sites and the probability of a splicing event at such sites. Additional splice prediction tools include RegRNA, ESEfinder, and MIT splice predictor. Splice site prediction tools such as GeneSplicer has been trained and/or tested successfully on databases for different species, such as human, Drosophila melanogaster, Plasmodium falciparum, Arabidopsis thaliana, and rice. In some embodiments, different prediction tools may be adapted for different extents on different database and/or for different species. In some embodiments, the one or more prediction tools are selected based upon their utility in certain database and/or for certain species. See, e.g., Saxonov et al., (2000) Nucleic Acids Res., 28, 185-190.

In some embodiments, one or more splice site prediction tools are used to determine potential splice donor and/or acceptor sites. In some embodiments, splice site prediction tools that can be run locally; that can be retrained with a set of data at the user site; that can use databases for particular species (such as human), that can be compiled for multiple platforms, that allow real-time predictions for sequence selections, and/or that is an OSI certified open source software such that particular tools or plugins can be modified, can be employed. Exemplary tools that can be employed include NNSplice, GeneSplicer or both.

In some aspects, the splice site prediction tools can be used to identify a list of potential splice donor and/or splice acceptor sites in a sequence such as a polynucleotide sequence containing transgene sequences. In some aspects, the prediction tools also can generate one or more prediction scores for one or more sequences in the polynucleotide, that can indicate the likelihoods of the one or more sequences being a splice donor or acceptor site sequence.

In some embodiments, the prediction score for a particular splice site is compared with a threshold score or reference score to determine or identify a particular splice sites that are candidate for elimination or removal. For example, in some embodiments, the predicted splice site is identified as a potential splice site when the prediction score is greater or no less than the threshold score or reference score. In some aspects, considerations for eliminating or removing a particular splice site include the prediction score as compared to a reference score or a threshold score; and whether a particular splice site is desired or intentional (for example, when the splicing event is more advantageous or is required for regulation of transcription and/or translation). In some aspects, the likelihood that the resulting splice variant loses the desired function or has compromised function can also be considered when determining particular donor and/or acceptor sites for elimination or removal. In some aspects, the one or more potential splice donor and/or splice acceptor sites exhibit a score about or at least about 0.7, 0.75, 0.8, 0.85, 0.9, 0.95, or 1.0 (e.g., on a scale with a maximum of 1.0) of a splice event or probability of a splice event, and the site can be a candidate for splice site elimination or removal. In some aspects, the score, e.g., used by GeneSplicer, at the one or more potential splice donor and/or splice site is based on the difference between the log-odds score returned for that sequence by the true Markov model and the score is computed by the false Markov model. In particular embodiments, the splice donor sites and splice acceptor sites are evaluated independently, or individually. In some embodiments, splice donor sites and splice acceptor sites are evaluated as a splice donor/acceptor pair.

In some embodiments, one or more splice donor and/or splice acceptor site(s), such as the potential splice donor and/or acceptor sites that may be involved in a cryptic splicing event that is not desired or that results in undesired RNA heterogeneity, is eliminated. In some embodiments, eliminating one or more splice sites comprises modifying one or more nucleotides (e.g., by substitution or replacement) in, at, containing or near the splice donor and/or acceptor sites that are candidates for removal. In some aspects, a particular nucleotide within a codon that is at, contains or is near the splice site is modified (e.g., substituted or replaced). In some aspects, the modification (such as substitution or replacement) retains or preserves the amino acid encoded by the particular codon at the site, at the same time removing the potential splice donor and/or acceptor sites.

In some embodiments, the codon at or near the splice site for modification comprises one or more codons that involve one or both of the two nucleotides at the potential splice site (in some cases referred to as “splice site codon”). When the potential splicing is predicted to occur between two nucleotides in a codon, the codon is the only splice site codon for this splice site. If the potential splicing is predicted to occur between two adjacent codons, for example, between the last nucleotide of the first codon and the first nucleotide of the next codon, the two codons are splice site codons. For example, for splice sites that are predicted to be at boundaries of two codons, the two adjacent codons can be candidates for nucleotide modification. In some embodiments, the one or more codons comprise one splice site codon. In some embodiments, the one or more codons comprise both splice site codons. In some embodiments, a potential splice donor site is eliminated by modifying one or both splice site codons. In some embodiments, a potential splice acceptor donor site is eliminated by modifying one or both splice site codons. In some embodiments, the one or both codons at the splice site is not modified, for example, when there are no synonymous codon for the splice site codon. In some embodiments, if there are no synonymous codons available for the particular splice site codon, one or more nucleotides in a nearby codon can be modified. In some embodiments, one or more codons that are modified include a splice site codon, wherein the modification comprises changing one or both nucleotides at the splice site to a different nucleotide or different nucleotides. In some embodiments, In some embodiments, the splice donor site is eliminated by modifying one or both splice site codons, wherein the modification does not change one or two of the nucleotides of the at the splice site to a different nucleotide, but a nearby nucleotide, e.g., a part of a codon adjacent to the splice site, is modified. In some embodiments, the nearby or adjacent nucleotides that can be modified include modification of a nucleotide that is a part of a nearby or adjacent codon, such as a codon that is within one, two, three, four, five, six, seven, eight, nine or ten codons upstream or downstream of the splice site codon.

In some cases, polynucleotides can be manually modified, while preserving the encoded amino acid sequence, to reduce the probability of a predicted splice site. In some embodiments, one or more of the predicted splice sites having at least 80%, 85%, 90%, or 95% probability of a splice site are manually modified to reduce the probability of the splicing event. In some embodiments, the one or more modification(s) is/are by nucleotide replacement or substitution of 1, 2, 3, 4, 5, 6 or 7 nucleotides. In some embodiments, the modification(s) is/are at the junction of the splice donor site or are at the junction of the splice acceptor site. In some embodiments, at least one of the one or more nucleotide modifications is within 1, 2, 3, 4, 5, 6, 7, 8, 9 or 10 residues of the splice site junction of the splice acceptor and/or splice donor site. In some embodiments, libraries of modified nucleic acid sequences can be generated with reduced probability of cryptic splice sites. In some embodiments, splice donor sites and splice acceptor sites are evaluated as a splice donor/acceptor pair. In particular embodiments, the splice donor sites and splice acceptor sites are evaluated independently, or individually, and not part as a splice donor/acceptor pair. In some embodiments, one or more predicted splice sites are not eliminated. In some embodiments, splice sites, such as known or predicted splice sites, within the promoter region of the transcript are not eliminated.

In some embodiments, one or more potential donor splice site is eliminated by modifying one or two splice site codons or one or more nearby or adjacent codons (for example, if a synonymous codon is not available for the splice site codon). In some embodiments, one or more potential acceptor splice site is eliminated by modifying one or two splice site codons or one or more nearby or adjacent codons (for example, if a synonymous codon is not available for the splice site codon). In some embodiments, the nearby or adjacent codon that is subject to modification include a codon that is within one, two, three, four, five, six, seven, eight, nine or ten codons upstream or downstream of the splice site codon, such as a codon that is within one, two or three codons from the splice site. In some embodiments, a potential branch site for splicing is removed or eliminated. In some aspects, a nucleotide within the codon at or near the branch site can be modified, e.g., substituted or replaced, to eliminate cryptic splicing and/or reduce RNA heterogeneity. In some embodiments, the modification of the one or more nucleotides can involve a substitution or replacement of one of the nucleotides that may be involved in splicing (such as at the splice donor site, splice acceptor site or splice branch site), such that the amino acid encoded by the codon is preserved, and the nucleotide substitution or replacement does not change the polypeptide sequence that is encoded by the polynucleotide. In some cases, the third position in the codon is more degenerate than the other two positions. Thus, various synonymous codons can encode a particular amino acid (see, e.g., Section F.1. above). In some embodiments, the modification includes replacing the codon with a synonymous codon used in the species of the cell into which the polynucleotide is introduced (e.g., human). In some embodiments, the species is human. In some embodiments, the one or more codon is replaced with a corresponding synonymous codons that the most frequently used in the species or synonymous codons that have a similar frequency of usage (e.g., most closest frequency of usage) as the corresponding codon (see, e.g., Section F.1. above).

In some embodiments, the transgene candidacy for the removal of splice sites is assessed, after initial proposed modification. In some aspects, the proposed modification can be evaluated again, to assess the proposed modification and identify any further potential splice sites after modification and/or codon optimization. In some aspects, after modifying the sequence for codon optimization and/or splice site removal, one or more further assessment of a sequence, such as a revised or modified nucleic acid sequence, is carried out to further evaluate for splice site removal, such as cryptic splice sites, using the same or one or more other or additional splice site prediction tool(s). In some aspects, proposed modifications are considered for subsequent steps, and iterative optimization can be used. In some aspects, the methods any of the identification and/or modification steps may be repeated, for example, until heterogeneity of the transcript is reduced compared to the heterogeneity of the transcript as initially determined. In some embodiments, a further or a different modification, such as with a different nucleotide replacement at the same codon or a modification at a different position or codon, can be done after an iterative evaluation and assessment. In some embodiments, corresponding different synonymous codon can be used, such as the second most frequently used in the particular species or a codon that has a similar frequency of usage (e.g., the next closest frequency of usage) as the corresponding codon (see, e.g., Section F.1 above).

In some aspects, a proposed modification can be further evaluated, for example, to assess whether the modification generates an undesired or additional restriction site in the polynucleotide. In some aspects, an additional restriction site may not be desired, and a further or a different modification (e.g., with a different nucleotide replacement at the same codon or a modification at a different position or codon) can be considered. In some aspects, particular restriction site, such as a designated restriction site, is avoided. In some aspects, if the modification does not substantially reduce the splice site prediction score, an additional or alternative modification can be proposed. In some embodiments, the splice site prediction score can be is reduced or lowered by at least about 5%, 10%, 15%, 20%, 25%, 30%, 35%, 40%, 45%, 50%, 55%, 60%, 65%, 70% or 75%, after one or more iteration of the methods.

In some embodiments, a computer system can be used to execute one or more steps, tools, functions, processes or scripts. In some embodiments, the splice site prediction, evaluation and modification for elimination or removal of a splice site can be performed by computer implemented methods and/or by methods which include steps that are computer implemented steps. In some embodiments, comparison of the sequences to a known database, calculating a splice site prediction score, determining potential nucleotide modifications, codon optimization and/or any one of the iterative steps can be implemented by a computer or using a computer-implemented steps, tools, functions, processes or scripts. In particular embodiments, a computer system comprising a processor and memory is provided, wherein the memory contains instructions operable to cause the processor to carry out any one or more of steps of the methods provided herein. In some embodiments, steps, functions, processes or scripts are performed computationally, e.g., performed using one or more computer programs and/or via the use of computational algorithms

Exemplary steps, functions, processes or scripts for identifying and/or removing possible splice sites include one or more steps of: selecting sequence, writing FASTA format sequences, loading codon table (e.g., from www.kazusa.or.jp/codon, running GeneSplicer, loading predictions, parsing codons, determining overlaps in prediction, identifying next highest usage synonymous codon, reviewing for restriction site, creating annotations or assessing other codons. Particular steps can assess both forward and reverse strands. In some aspects, previously annotated splice site modifications can also be considered, to allow for iterative optimization. In some embodiments, any one or more of the steps, functions, processes or scripts can be repeated.

In some embodiments, a provided polynucleotide encoding a CAR provided herein, or a construct provided herein, includes modifications to remove one or more splice donor and/or acceptor site that may contribute to splice events and/or reduced expression and/or increased RNA heterogeneity.

### 3. Other Features

Also provided are vectors containing the polynucleotides and host cells containing the vectors, e.g., for producing the chimeric antigen receptors. Also provided are methods for producing the chimeric antigen receptors. The nucleic acid may encode a chimeric antigen receptor comprising a V_{L} region and/or a V_{H} region of an antibody (e.g., the light and/or heavy chains of the antibody). The nucleic acid may encode one or more amino binding domains (e.g., a CD19-binding domain and a BAFF-R-binding domain) each comprising a V_{L} region and/or a V_{H} region of an antibody (e.g., the light and/or heavy chains of the antibody). In a further embodiment, one or more vectors (e.g., expression vectors) comprising such polynucleotides are provided. In a further embodiment, a host cell comprising such polynucleotides is provided. In one such embodiment, a host cell comprises (e.g., has been transformed with) a vector comprising a nucleic acid that encodes chimeric antigen receptor comprising the V_{H} region of an antibody. In another such embodiment, a host cell comprises (e.g., has been transformed with) (1) a vector comprising a nucleic acid that encodes a chimeric antigen receptor comprising the V_{L} region of the antibody and the V_{H} region of the antibody, or (2) a vector comprising a nucleic acid that encodes a chimeric antigen receptor comprising a first antibody and a second antibody. In some embodiments, a host cell comprises (e.g., has been transformed with) one or more vectors comprising one or more nucleic acid that encodes one or more chimeric antigen receptors. In some embodiments, one or more such host cells are provided. In some embodiments, a composition containing one or more such host cells are provided. In some embodiments, the one or more host cells can express different chimeric antigen receptors, or the same chimeric antigen receptor. In some embodiments, each of the host cells can express more than one chimeric antigen receptor.

Also provided are methods of making the bispecific chimeric antigen receptors that bind to CD19 and BAFF-R. For recombinant production of the chimeric receptors, a nucleic acid sequence encoding a chimeric receptor antibody, e.g., as described herein, may be isolated and inserted into one or more vectors for further cloning and/or expression in a host cell. Such nucleic acid sequences may be readily isolated and sequenced using conventional procedures (e.g., by using oligonucleotide probes that are capable of binding specifically to genes encoding the heavy and light chains of the antibody). In some embodiments, a method of making the bispecific chimeric antigen receptor is provided, wherein the method comprises culturing a host cell comprising a nucleic acid sequence encoding the antibodies (i.e., the CD19-binding domain and the BAFF-R-binding domain), as provided above, under conditions suitable for expression of the receptor.

In some embodiments, a method of making a cellular composition comprising cells expressing the bispecific chimeric antigen receptor is provided.

In addition to prokaryotes, eukaryotic microbes such as filamentous fungi or yeast are suitable cloning or expression hosts for antibody-encoding vectors, including fungi and yeast strains whose glycosylation pathways have been modified to mimic or approximate those in human cells, resulting in the production of an antibody with a partially or fully human glycosylation pattern. See Gerngross, Nat. Biotech. 22:1409-1414 (2004), and Li et al., Nat. Biotech. 24:210-215 (2006).

Exemplary eukaryotic cells that may be used to express polypeptides include, but are not limited to, COS cells, including COS 7 cells; 293 cells, including 293-6E cells; CHO cells, including CHO-S, DG44. Lec13 CHO cells, and FUT8 CHO cells; PER.C6® cells; and NSO cells. In some embodiments, the antibody heavy chains and/or light chains (e.g., V_{H} region and/or VL region) may be expressed in yeast (see, e.g., U.S. Publication No. US 2006/0270045 A1). In some embodiments, a particular eukaryotic host cell is selected based on its ability to make desired post-translational modifications to the heavy chains and/or light chains (e.g., V_{H} region and/or V_{L} region). For example, in some embodiments, CHO cells produce polypeptides that have a higher level of sialylation than the same polypeptide produced in 293 cells. In particular examples immune cells, such as human immune cells are used to express the provided polypeptides encoding chimeric antigen receptors. In some examples, the immune cells are T cells, such as CD4+ and/or CD8+ immune cells.

### III. ENGINEERED CELLS

Also provided are cells such as engineered cells that contain a recombinant receptor (e.g., a chimeric antigen receptor) such as one that contains an extracellular binding domain including both a BAFF-R-binding domain and a CD19-binding domain as provided herein. Also provided are populations of such cells, compositions containing such cells and/or enriched for such cells, such as in which cells expressing the BAFF-R-binding domain and the CD19 binding domain make up at least 50, 60, 70, 80, 90, 91, 92, 93, 94, 95, 96, 97, 98, 99, or more percent of the total cells in the composition or cells of a certain type such as T cells, CD8+ cells or CD4+ cells.

Also provided are cells such as engineered cells that are engineered to contain a recombinant receptor (e.g., a CAR) comprising a BAFF-R-binding domain and a CD19-binding domain. In some embodiments, the recombinant receptor is a tandem CAR comprising a BAFF-R-binding domain and CD19-binding domain. Exemplary BAFF-R-binding domains are described in Section I.

In some embodiments, the engineered cells provided herein can be combined with one or more engineered cell population(s) expressing one or more other recombinant receptor(s). Such engineered cell populations can be formulated in the same or separate compositions. Among the compositions are pharmaceutical compositions and formulations for administration, such as for adoptive cell therapy. Also provided are therapeutic methods for administering any of the cells or compositions provided herein to subjects, e.g., patients.

Also provided are genetically engineered cells expressing the recombinant receptors containing the antibodies, e.g., cells containing the CARs. The cells generally are eukaryotic cells, such as mammalian cells, and typically are human cells. In some embodiments, the cells are derived from the blood, bone marrow, lymph, or lymphoid organs, are cells of the immune system, such as cells of the innate or adaptive immunity, e.g., myeloid or lymphoid cells, including lymphocytes, typically T cells and/or NK cells. Other exemplary cells include stem cells, such as multipotent and pluripotent stem cells, including induced pluripotent stem cells (iPSCs). The cells typically are primary cells, such as those isolated directly from a subject and/or isolated from a subject and frozen.

In some embodiments, the cells are T cells. In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. In some embodiments, provided herein is a T cell comprising a provided bispecific CAR. In some embodiments the T cell is a CD4+ T cell. In some embodiments, the T cell is a CD8+ T cell.

With reference to the subject to be treated, the cells may be allogeneic and/or autologous. Among the methods include off-the-shelf methods. In some aspects, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, as described herein, and re-introducing them into the same patient, before or after cryopreservation.

Among the sub-types and subpopulations of T cells and/or of CD4⁺ and/or of CD8⁺ T cells are naïve T (T_{N}) cells, effector T cells (T_{EFF}), memory T cells and sub-types thereof, such as stem cell memory T (T_{SCM}), central memory T (T_{CM}), effector memory T (T_{EM}), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, the cells are natural killer (NK) cells. In some embodiments, the cells are monocytes or granulocytes, e.g., myeloid cells, macrophages, neutrophils, dendritic cells, mast cells, eosinophils, and/or basophils.

In some embodiments, the cells include one or more polynucleotides introduced via genetic engineering, and thereby express recombinant or genetically engineered products of such polynucleotides. In some embodiments, the polynucleotides are heterologous, i.e., normally not present in a cell or sample obtained from the cell, such as one obtained from another organism or cell, which for example, is not ordinarily found in the cell being engineered and/or an organism from which such cell is derived. In some embodiments, the polynucleotides are not naturally occurring, such as a polynucleotide not found in nature, including one comprising chimeric combinations of polynucleotides encoding various domains from multiple different cell types. In some embodiments, the cells (e.g., engineered cells) comprise a vector (e.g., a viral vector, expression vector, etc.) as described herein such as a vector comprising a nucleic acid comprising a nucleic acid encoding a recombinant receptor described herein.

### A. Vectors and Methods for Genetic Engineering

Also provided are methods, nucleic acids, compositions, and kits, for expressing the bispecific recombinant receptors (e.g., CARs) and for producing the genetically engineered cells expressing said receptors. In some embodiments, one or more recombinant receptors (e.g., CARs) can be genetically engineered into cells or plurality of cells. The genetic engineering generally involves introduction of a nucleic acid encoding the recombinant or engineered component into the cell, such as by retroviral transduction, transfection, or transformation.

In some embodiments, gene transfer is accomplished by first stimulating the cell, such as by combining it with a stimulus that induces a response such as proliferation, survival, and/or activation, e.g., as measured by expression of a cytokine or activation marker, followed by transduction of the activated cells, and expansion in culture to numbers sufficient for clinical applications.

In some contexts, overexpression of a stimulatory factor (for example, a lymphokine or a cytokine) may be toxic to a subject. Thus, in some contexts, the engineered cells include gene segments that cause the cells to be susceptible to negative selection in vivo, such as upon administration in adoptive immunotherapy. For example in some aspects, the cells are engineered so that they can be eliminated as a result of a change in the in vivo condition of the patient to which they are administered. The negative selectable phenotype may result from the insertion of a gene that confers sensitivity to an administered agent, for example, a compound. Negative selectable genes include the Herpes simplex virus type I thymidine kinase (HSV-I TK) gene (Wigler et al., Cell 2:223, 1977) which confers ganciclovir sensitivity; the cellular hypoxanthine phosphoribosyltransferase (HPRT) gene, the cellular adenine phosphoribosyltransferase (APRT) gene, bacterial cytosine deaminase, (Mullen et al., Proc. Natl. Acad. Sci. USA. 89:33 (1992)).

In some aspects, the cells further are engineered to promote expression of cytokines or other factors. Various methods for the introduction of genetically engineered components, e.g., bispecific CARs, are well known and may be used with the provided methods and compositions. Exemplary methods include those for transfer of polynucleotides encoding the receptors, including via viral, e.g., retroviral or lentiviral, transduction, transposons, and electroporation.

In some embodiments, recombinant polynucleotides are transferred into cells using recombinant infectious virus particles, such as, e.g., vectors derived from simian virus 40 (SV40), adenoviruses, or adeno-associated virus. In some embodiments, recombinant polynucleotides are transferred into T cells using recombinant lentiviral vectors, such as HIV-1 lentivirus-based vectors (lentivectors; see, e.g., Amado et al., Science. 1999 Jul. 30; 285(5428): 674-676), or retroviral vectors, such as gamma-retroviral vectors (see, e.g., Koste et al. (2014) Gene Therapy 2014 Apr. 3. doi: 10.1038/gt.2014.25; Carlens et al. (2000) Exp Hematol 28(10): 1137-46; Alonso-Camino et al. (2013) Mol Ther Nucl Acids 2, e93; Park et al., Trends Biotechnol. 2011 Nov. 29(11): 550-557).

In some embodiments, the retroviral vector or lentiviral vector has a long terminal repeat sequence (LTR). In some embodiments the vector is derived from the Moloney murine leukemia virus (MoMLV), myeloproliferative sarcoma virus (MPSV), murine embryonic stem cell virus (MESV), murine stem cell virus (MSCV), spleen focus forming virus (SFFV), human immunodeficiency virus type 1 (HIV-1) or human immunodeficiency virus type 2 (HIV-2/SIV). In some embodiments, the vectors are self-inactivating (SIN). In some embodiments, the vectors are conditionally replicating (mobilizable) vectors. Most lentiviral vectors are derived from human, feline or simian lentiviruses. Most retroviral vectors are derived from murine retroviruses. In some embodiments, the lentiviruses or retroviruses include those derived from any avian or mammalian cell source. The lentiviruses or retroviruses typically are amphotropic, meaning that they are capable of infecting host cells of several species, including humans. In one embodiment, the gene to be expressed replaces the retroviral gag, pol and/or env sequences. Methods of lentiviral transduction are known. Exemplary methods are described in, e.g., Wang et al. (2012) J. Immunother. 35(9): 689-701; Cooper et al. (2003) Blood. 101:1637-1644; Verhoeyen et al. (2009) Methods Mol Biol. 506:97-114; and Cavalieri et al. (2003) Blood. 102(2): 497-505. A number of illustrative retroviral systems have also been described (e.g., Amado et al., (1999) Science 285(5428): 674-676, U.S. Pat. Nos. 5,219,740; 6,207,453; 5,219,740; Miller and Rosman (1989) BioTechniques 7:980-990; Miller (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3:102-109).

In some embodiments, recombinant polynucleotides are transferred into T cells via electroporation (see, e.g., Chicaybam et al, (2013) PLOS ONE 8(3): e60298 and Van Tedeloo et al. (2000) Gene Therapy 7(16): 1431-1437). In some embodiments, recombinant polynucleotides are transferred into T cells via transposition (see, e.g., Manuri et al. (2010) Hum Gene Ther 21(4): 427-437; Sharma et al. (2013) Molec Ther Nucl Acids 2, e74; and Huang et al. (2009) Methods Mol Biol 506:115-126). Other methods of introducing and expressing genetic material in immune cells include calcium phosphate transfection (e.g., as described in Current Protocols in Molecular Biology, John Wiley & Sons, New York. N.Y.), protoplast fusion, cationic liposome-mediated transfection; tungsten particle-facilitated microparticle bombardment (Johnston (1990) Nature 346:776-777); and strontium phosphate DNA co-precipitation (Brash et al., (1987) Mol. Cell Biol. 7:2031-2034). Other approaches and vectors for transfer of the polynucleotides encoding the recombinant products are those described, e.g., in WO2014055668, and U.S. Pat. No. 7,446,190.

Among additional polynucleotides, e.g., genes for introduction are those to improve the outcome of therapy, such as by promoting viability and/or function of transferred cells; genes to provide a genetic marker for selection and/or evaluation of the cells, such as to assess in vivo survival or localization; genes to improve safety, for example, by making the cell susceptible to negative selection in vivo as described by Lupton S. D. et al., Mol. and Cell Biol., 11:6 (1991); and Riddell et al., Human Gene Therapy 3:319-338 (1992); see also the publications of PCT/US91/08442 and PCT/US94/05601 by Lupton et al. describing the use of bifunctional selectable fusion genes derived from fusing a dominant positive selectable marker with a negative selectable marker. See, e.g., Riddell et al., U.S. Pat. No. 6,040,177, at columns 14-17.

In some embodiments, one or more recombinant receptors (e.g., CARs) can be genetically engineered to be expressed in cells or plurality of cells. In some embodiments, a recombinant receptor is a CAR. In some embodiments, the CAR comprises two antigen-binding domains. In some embodiments, the CAR comprises a BAFF-R-binding domain that binds to BAAFF-R (e.g., human BAFF-R) and a CD19-binding domain that binds to CD19 (e.g., human CD19). In some embodiments, the BAFF-R-binding domain and the CD19-binding domain of the CAR are separated by a linker, such as a polypeptide linker.

In some embodiments the vector or construct can contain a promoter and/or enhancer or regulatory elements to regulate expression of the encoded recombinant receptor. In some examples the promoter and/or enhancer or regulatory elements can be condition-dependent promoters, enhancers, and/or regulatory elements. In some examples these elements drive expression of the transgene. In some examples, the CAR transgene can be operatively linked to a promoter, such as an EF1alpha promoter with an HTLV1 enhancer (SEQ ID NO: 105). In some examples, the CAR transgene is operatively linked to a Woodchuck Hepatitis Virus (WHP) Posttranscriptional Regulatory Element (WPRE; SEQ ID NO: 106), located downstream of the transgene.

In some embodiments, the vector or construct can contain a single promoter that drives the expression of one or more nucleic acid molecules. In some embodiments, such nucleic acid molecules, e.g., transcripts, can be multicistronic (bicistronic or tricistronic, see e.g., U.S. Pat. No. 6,060,273). For example, in some embodiments, transcription units can be engineered as a bicistronic unit containing an IRES (internal ribosome entry site), which allows coexpression of gene products (e.g., encoding a first and second chimeric receptor) by a message from a single promoter.

Alternatively, in some cases, a single promoter may direct expression of an RNA that contains, in a single open reading frame (ORF), two or three genes (e.g., encoding a first and second binding molecules, e.g., antibody recombinant receptor) separated from one another by sequences encoding a self-cleavage peptide (e.g., 2A cleavage sequences) or a protease recognition site (e.g., furin). The ORF thus encodes a single polypeptide, which, either during (in the case of T2A) or after translation, is cleaved into the individual proteins. In some cases, the peptide, such as T2A, can cause the ribosome to skip (ribosome skipping) synthesis of a peptide bond at the C-terminus of a 2A element, leading to separation between the end of the 2A sequence and the next peptide downstream (see, for example, de Felipe. Genetic Vaccines and Ther. 2:13 (2004) and deFelipe et al. Traffic 5:616-626 (2004)). Many 2A elements are known. Examples of 2A sequences that can be used in the methods and polynucleotides disclosed herein, without limitation, 2A sequences from the foot-and-mouth disease virus (F2A, e.g., SEQ ID NO: 107 or 108), equine rhinitis A virus (E2A, e.g., SEQ ID NO: 109 or 110), Thosea asigna virus (T2A, e.g., SEQ ID NO: 111, 112, or 113), and porcine teschovirus-1 (P2A, e.g., SEQ ID NO: 114 or 115) as described in U.S. Patent Publication No. 20070116690. In some embodiments, the one or more different or separate promoters drive the expression of one or more nucleic acid molecules encoding the one or more binding molecules, e.g., recombinant receptors.

Any of the recombinant receptors provided herein, e.g., bispecific CARs binding to BAFF-R and CD19, can be encoded by polynucleotides containing one or more nucleic acid molecules encoding the receptors, in any combinations or arrangements. For example, one, two, three or more polynucleotides can encode one, two, three or more different receptors or domains. In some embodiments, one vector or construct contains nucleic acid molecules encoding one or more recombinant receptor(s), and a separate vector or construct contains nucleic acid molecules encoding an additional binding molecule, e.g., antibody and/or recombinant receptor.

In some embodiments, the nucleic acid molecules can also encode one or more surrogate marker(s), such as fluorescent protein (e.g., green fluorescent protein (GFP)) or a cell surface marker (e.g., a truncated surface marker such as truncated EGFR (tEGFR), which may be used to confirm transduction or engineering of the cell to express the receptor. For example, in some aspects, extrinsic marker genes are utilized in connection with engineered cell therapies to permit detection or selection of cells and, in some cases, also to promote cell suicide by ADCC. Exemplary marker genes include truncated epidermal growth factor receptor (EGFRt), which can be co-expressed with a transgene of interest (e.g., a CAR or TCR) in transduced cells (see, e.g., U.S. Pat. No. 8,802,374). EGFRt contains an epitope recognized by the antibody cetuximab (Erbitux®). For this reason, Erbitux® can be used to identify or select cells that have been engineered with the EGFRt construct, including in cells also co-engineered with another recombinant receptor, such as a chimeric antigen receptor (CAR).

In some embodiments, the nucleic acid encoding the binding molecules further contain contains a nucleic acid sequence encoding one or more marker(s). In some embodiments, the one or more marker(s) is a transduction marker, surrogate marker and/or a selection marker.

In some embodiments, the marker is a transduction marker or a surrogate marker. A transduction marker or a surrogate marker can be used to detect cells that have been introduced with the polynucleotide, e.g., a polynucleotide encoding the bispecific CARs. In some embodiments, the transduction marker can indicate or confirm modification of a cell. In some embodiments, the surrogate marker is a protein that is made to be co-expressed on the cell surface with the bispecific CAR. In particular embodiments, such a surrogate marker is a surface protein that has been modified to have little or no activity. In certain embodiments, the surrogate marker is encoded on the same polynucleotide that encodes the bispecific CAR. In some embodiments, the nucleic acid sequence encoding the recombinant receptor is operably linked to a nucleic acid sequence encoding a marker, optionally separated by an internal ribosome entry site (IRES), or a nucleic acid encoding a self-cleaving peptide or a peptide that causes ribosome skipping, such as a 2A sequence, such as a T2A, a P2A, an E2A or an F2A. Extrinsic marker genes may in some cases be utilized in connection with engineered cell to permit detection or selection of cells and, in some cases, also to promote cell suicide.

Exemplary surrogate markers can include truncated forms of cell surface polypeptides, such as truncated forms that are non-functional and to not transduce or are not capable of transducing a signal or a signal ordinarily transduced by the full-length form of the cell surface polypeptide, and/or do not or are not capable of internalizing. Exemplary truncated cell surface polypeptides including truncated forms of growth factors or other receptors such as a truncated human epidermal growth factor receptor 2 (tHER2), a truncated epidermal growth factor receptor (tEGFR, exemplary tEGFR sequence set forth in SEQ ID NO: 116-118) or a prostate-specific membrane antigen (PSMA) or modified form thereof. tEGFR may contain an epitope recognized by the antibody cetuximab (Erbitux®) or other therapeutic anti-EGFR antibody or binding molecule, which can be used to identify or select cells that have been engineered with the tEGFR construct and an encoded exogenous protein, and/or to eliminate or separate cells expressing the encoded exogenous protein. See U.S. Pat. No. 8,802,374 and Liu et al., Nature Biotech. 2016 April; 34(4): 430-434). In some aspects, the marker, e.g. surrogate marker, includes all or part (e.g., truncated form) of CD34, a NGFR, a CD19 or a truncated CD19, e.g., a truncated non-human CD19, or epidermal growth factor receptor (e.g., tEGFR).

In some embodiments, the marker is or comprises a fluorescent protein, such as green fluorescent protein (GFP), enhanced green fluorescent protein (EGFP), such as super-fold GFP (sfGFP), red fluorescent protein (RFP), such as tdTomato, mCherry, mStrawberry, AsRed2, DsRed or DsRed2, cyan fluorescent protein (CFP), blue green fluorescent protein (BFP), enhanced blue fluorescent protein (EBFP), and yellow fluorescent protein (YFP), and variants thereof, including species variants, monomeric variants, and codon-optimized and/or enhanced variants of the fluorescent proteins. In some embodiments, the marker is or comprises an enzyme, such as a luciferase, the lacZ gene from E. coli, alkaline phosphatase, secreted embryonic alkaline phosphatase (SEAP), chloramphenicol acetyl transferase (CAT). Exemplary light-emitting reporter genes include luciferase (luc), β-galactosidase, chloramphenicol acetyltransferase (CAT), β-glucuronidase (GUS) or variants thereof.

In some embodiments, the marker is a selection marker. In some embodiments, the selection marker is or comprises a polypeptide that confers resistance to exogenous agents or drugs. In some embodiments, the selection marker is an antibiotic resistance gene. In some embodiments, the selection marker is an antibiotic resistance gene confers antibiotic resistance to a mammalian cell. In some embodiments, the selection marker is or comprises a Puromycin resistance gene, a Hygromycin resistance gene, a Blasticidin resistance gene, a Neomycin resistance gene, a Geneticin resistance gene or a Zeocin resistance gene or a modified form thereof.

In some embodiments, the nucleic acid encoding the marker is operably linked to a polynucleotide encoding for a linker sequence, such as a cleavable linker sequence, e.g., a T2A. For example, a marker, and optionally a linker sequence, can be any as disclosed in PCT Pub. No. WO2014031687. For example, the marker can be a truncated EGFR (tEGFR) that is, optionally, linked to a linker sequence, such as a T2A cleavable linker sequence. An exemplary polypeptide for a truncated EGFR (e.g. tEGFR) comprises the sequence of amino acids set forth in SEQ ID NO: 116-118 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 116-118.

In some embodiments, the marker is a molecule, e.g., cell surface protein, not naturally found on T cells or not naturally found on the surface of T cells, or a portion thereof.

In some embodiments, the molecule is a non-self-molecule, e.g., non-self-protein, i.e., one that is not recognized as “self” by the immune system of the host into which the cells will be adoptively transferred.

In some embodiments, the marker serves no therapeutic function and/or produces no effect other than to be used as a marker for genetic engineering, e.g., for selecting cells successfully engineered. In other embodiments, the marker may be a therapeutic molecule or molecule otherwise exerting some desired effect, such as a ligand for a cell to be encountered in vivo, such as a costimulatory or immune checkpoint molecule to enhance and/or dampen responses of the cells upon adoptive transfer and encounter with ligand.

Also provided are compositions containing one or more of the nucleic acid molecules, vectors or constructs, such as any described above. In some embodiments, the nucleic acid molecules, vectors, constructs or compositions can be used to engineer cells, such as T cells, to express any of the binding molecules, e.g., antibody or recombinant receptor, and/or the additional binding molecules.

### B. Preparation of Cells for Engineering

In some embodiments, preparation of the engineered cells includes one or more culture and/or preparation steps. The cells for introduction of the recombinant receptor (e.g., CAR) may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered.

Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g., transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, non-human primate, or pig.

In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contain cells other than red blood cells and platelets.

In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some aspects, a washing step is accomplished a semi-automated “flow-through” centrifuge (for example, the Cobe 2991 cell processor, Baxter) according to the manufacturer's instructions. In some aspects, a washing step is accomplished by tangential flow filtration (TFF) according to the manufacturer's instructions. In some embodiments, the cells are resuspended in a variety of biocompatible buffers after washing, such as, for example, Ca++/Mg++ free PBS. In certain embodiments, components of a blood cell sample are removed and the cells directly resuspended in culture media.

In some embodiments, the methods include density-based cell separation methods, such as the preparation of white blood cells from peripheral blood by lysing the red blood cells and centrifugation through a Percoll or Ficoll gradient.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

For example, in some aspects, specific subpopulations of T cells, such as cells positive or expressing high levels of one or more surface markers, e.g., CD28+, CD62L+, CCR7+, CD27+, CD127+, CD4+, CD8+, CD45RA+, and/or CD45RO+ T cells, are isolated by positive or negative selection techniques.

For example, CD3+, CD28+ T cells can be positively selected using anti-CD3/anti-CD28 conjugated magnetic beads (e.g., DYNABEADS® M-450 CD3/CD28 T Cell Expander, MACSiBeads™, etc.).

In some embodiments, isolation is carried out by enrichment for a particular cell population by positive selection, or depletion of a particular cell population, by negative selection. In some embodiments, positive or negative selection is accomplished by incubating cells with one or more antibodies or other binding agent that specifically bind to one or more surface markers expressed or expressed (marker+) at a relatively higher level (marker^{high}) on the positively or negatively selected cells, respectively.

In some embodiments, T cells are separated from a PBMC sample by negative selection of markers expressed on non-T cells, such as B cells, monocytes, or other white blood cells, such as CD14. In some aspects, a CD4+ or CD8+ selection step is used to separate CD4+ helper and CD8+ cytotoxic T cells. Such CD4+ and CD8+ populations can be further sorted into sub-populations by positive or negative selection for markers expressed or expressed to a relatively higher degree on one or more naive, memory, and/or effector T cell subpopulations.

In some embodiments, CD8+ cells are further enriched for or depleted of naive, central memory, effector memory, and/or central memory stem cells, such as by positive or negative selection based on surface antigens associated with the respective subpopulation. In some embodiments, enrichment for central memory T (T_{CM}) cells is carried out to increase certain features, such as to improve long-term survival, expansion, and/or engraftment following administration, which in some aspects is particularly robust in such sub-populations (see Terakura et al. (2012) Blood. 1:72-82; Wang et al. (2012) J Immunother. 35(9): 689-701). In some embodiments, combining TCM-enriched CD8+ T cells and CD4+ T cells further enhances response.

In embodiments, memory T cells are present in both CD62L+ and CD62L-subsets of CD8+ peripheral blood lymphocytes. PBMC can be enriched for or depleted of CD62L-CD8+ and/or CD62L+CD8+ fractions, such as using anti-CD8 and anti-CD62L antibodies.

In some embodiments, the enrichment for central memory T (T_{CM}) cells is based on positive or high surface expression of CD45RO, CD62L, CCR7, CD28, CD3, and/or CD 127; in some aspects, it is based on negative selection for cells expressing or highly expressing CD45RA and/or granzyme B. In some aspects, isolation of a CD8+ population enriched for TCM cells is carried out by depletion of cells expressing CD4, CD14, CD45RA, and positive selection or enrichment for cells expressing CD62L. In one aspect, enrichment for central memory T (T_{CM}) cells is carried out starting with a negative fraction of cells selected based on CD4 expression, which is subjected to a negative selection based on expression of CD14 and CD45RA, and a positive selection based on CD62L. Such selections in some aspects are carried out simultaneously and in other aspects are carried out sequentially, in either order. In some aspects, the same CD4 expression-based selection step used in preparing the CD8+ cell population or subpopulation, also is used to generate the CD4+ cell population or sub-population, such that both the positive and negative fractions from the CD4-based separation are retained and used in subsequent steps of the methods, optionally following one or more further positive or negative selection steps.

In a particular example, a sample of PBMCs or other white blood cell sample is subjected to selection of CD4+ cells, where both the negative and positive fractions are retained. The negative fraction then is subjected to negative selection based on expression of CD14 and CD45RA, and positive selection based on a marker characteristic of central memory T cells, such as CD62L or CCR7, where the positive and negative selections are carried out in either order.

CD4+T helper cells are sorted into naïve, central memory, and effector cells by identifying cell populations that have cell surface antigens. CD4+ lymphocytes can be obtained by standard methods. In some embodiments, naive CD4+T lymphocytes are CD45RO−, CD45RA+, CD62L+, CD4+ T cells. In some embodiments, central memory CD4+ cells are CD62L+ and CD45RO+. In some embodiments, effector CD4+ cells are CD62L− and CD45RO−.

In one example, to enrich for CD4+ cells by negative selection, a monoclonal antibody cocktail typically includes antibodies to CD14, CD20, CD11b, CD16, HLA-DR, and CD8. In some embodiments, the antibody or binding partner is bound to a solid support or matrix, such as a magnetic bead or paramagnetic bead, to allow for separation of cells for positive and/or negative selection. For example, in some embodiments, the cells and cell populations are separated or isolated using immunomagnetic (or affinity magnetic) separation techniques (reviewed in Methods in Molecular Medicine, vol. 58: Metastasis Research Protocols, Vol. 2: Cell Behavior In vitro and In vivo, p 17-25 Edited by: S. A. Brooks and U. Schumacher @ Humana Press Inc., Totowa, NJ).

In some aspects, the sample or composition of cells to be separated is incubated with small, magnetizable or magnetically responsive material, such as magnetically responsive particles or microparticles, such as paramagnetic beads (e.g., such as Dynabeads® or MACS® beads). The magnetically responsive material, e.g., particle, generally is directly or indirectly attached to a binding partner, e.g., an antibody, that specifically binds to a molecule, e.g., surface marker, present on the cell, cells, or population of cells that it is desired to separate, e.g., that it is desired to negatively or positively select.

In some embodiments, the magnetic particle or bead comprises a magnetically responsive material bound to a specific binding member, such as an antibody or other binding partner. There are many well-known magnetically responsive materials used in magnetic separation methods. Suitable magnetic particles include those described in Molday, U.S. Pat. No. 4,452,773, and in European Patent Specification EP 452342 B, which are hereby incorporated by reference. Colloidal sized particles, such as those described in Owen U.S. Pat. No. 4,795,698, and Liberti et al., U.S. Pat. No. 5,200,084, are other examples.

The incubation generally is carried out under conditions whereby the antibodies or binding partners, or molecules, such as secondary antibodies or other reagents, which specifically bind to such antibodies or binding partners, which are attached to the magnetic particle or bead, specifically bind to cell surface molecules if present on cells within the sample.

In some aspects, the sample is placed in a magnetic field, and those cells having magnetically responsive or magnetizable particles attached thereto will be attracted to the magnet and separated from the unlabeled cells. For positive selection, cells that are attracted to the magnet are retained; for negative selection, cells that are not attracted (unlabeled cells) are retained. In some aspects, a combination of positive and negative selection is performed during the same selection step, where the positive and negative fractions are retained and further processed or subject to further separation steps.

In certain embodiments, the magnetically responsive particles are coated in primary antibodies or other binding partners, secondary antibodies, lectins, enzymes, or streptavidin. In certain embodiments, the magnetic particles are attached to cells via a coating of primary antibodies specific for one or more markers. In certain embodiments, the cells, rather than the beads, are labeled with a primary antibody or binding partner, and then cell-type specific secondary antibody- or other binding partner (e.g., streptavidin)-coated magnetic particles, are added. In certain embodiments, streptavidin-coated magnetic particles are used in conjunction with biotinylated primary or secondary antibodies.

In some embodiments, the magnetically responsive particles are left attached to the cells that are to be subsequently incubated, cultured and/or engineered; in some aspects, the particles are left attached to the cells for administration to a patient. In some embodiments, the magnetizable or magnetically responsive particles are removed from the cells. Methods for removing magnetizable particles from cells are known and include, e.g., the use of competing non-labeled antibodies, magnetizable particles or antibodies conjugated to cleavable linkers, etc. In some embodiments, the magnetizable particles are biodegradable.

In some embodiments, the affinity-based selection is via magnetic-activated cell sorting (MACS®) (Miltenyi Biotec, Auburn, CA). Magnetic Activated Cell Sorting (MACS®) systems are capable of high-purity selection of cells having magnetized particles attached thereto. In certain embodiments, MACS® operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they can be eluted and recovered. In certain embodiments, the non-target cells are labelled and depleted from the heterogeneous population of cells.

In certain embodiments, the isolation or separation is carried out using a system, device, or apparatus that carries out one or more of the isolation, cell preparation, separation, processing, incubation, culture, and/or formulation steps of the methods. In some aspects, the system is used to carry out each of these steps in a closed or sterile environment, for example, to minimize error, user handling and/or contamination. In one example, the system is a system as described in WO2009/072003 or US20110003380.

In some embodiments, the system or apparatus carries out one or more, e.g., all, of the isolation, processing, engineering, and formulation steps in an integrated or self-contained system, and/or in an automated or programmable fashion. In some aspects, the system or apparatus includes a computer and/or computer program in communication with the system or apparatus, which allows a user to program, control, assess the outcome of, and/or adjust various aspects of the processing, isolation, engineering, and formulation steps.

In some aspects, the separation and/or other steps is carried out using CliniMACS® system (Miltenyi Biotec), for example, for automated separation of cells on a clinical-scale level in a closed and sterile system. Components can include an integrated microcomputer, magnetic separation unit, peristaltic pump, and various pinch valves. The integrated computer in some aspects controls all components of the instrument and directs the system to perform repeated procedures in a standardized sequence. The magnetic separation unit in some aspects includes a movable permanent magnet and a holder for the selection column. The peristaltic pump controls the flow rate throughout the tubing set and, together with the pinch valves, ensures the controlled flow of buffer through the system and continual suspension of cells.

The CliniMACS® system in some aspects uses antibody-coupled magnetizable particles that are supplied in a sterile, non-pyrogenic solution. In some embodiments, after labelling of cells with magnetic particles the cells are washed to remove excess particles. A cell preparation bag is then connected to the tubing set, which in turn is connected to a bag containing buffer and a cell collection bag. The tubing set consists of pre-assembled sterile tubing, including a pre-column and a separation column, and are for single use only. After initiation of the separation program, the system automatically applies the cell sample onto the separation column. Labelled cells are retained within the column, while unlabeled cells are removed by a series of washing steps. In some embodiments, the cell populations for use with the methods described herein are unlabeled and are not retained in the column. In some embodiments, the cell populations for use with the methods described herein are labeled and are retained in the column. In some embodiments, the cell populations for use with the methods described herein are eluted from the column after removal of the magnetic field, and are collected within the cell collection bag.

In certain embodiments, separation and/or other steps are carried out using the CliniMACS Prodigy® system (Miltenyi Biotec). The CliniMACS Prodigy® system in some aspects is equipped with a cell processing unity that permits automated washing and fractionation of cells by centrifugation. The CliniMACS Prodigy® system can also include an onboard camera and image recognition software that determines the optimal cell fractionation endpoint by discerning the macroscopic layers of the source cell product. For example, peripheral blood may be automatically separated into erythrocytes, white blood cells and plasma layers. The CliniMACS Prodigy® system can also include an integrated cell cultivation chamber which accomplishes cell culture protocols such as, e.g., cell differentiation and expansion, antigen loading, and long-term cell culture. Input ports can allow for the sterile removal and replenishment of media and cells can be monitored using an integrated microscope (see, e.g., Klebanoff et al. (2012) J Immunother. 35(9): 651-660, Terakura et al. (2012) Blood. 1:72-82, and Wang et al. (2012) J Immunother. 35(9): 689-701).

In some embodiments, a cell population described herein is collected and enriched (or depleted) via flow cytometry, in which cells stained for multiple cell surface markers are carried in a fluidic stream. In some embodiments, a cell population described herein is collected and enriched (or depleted) via preparative scale (FACS)-sorting. In certain embodiments, a cell population described herein is collected and enriched (or depleted) by use of microelectromechanical systems (MEMS) chips in combination with a FACS-based detection system (see, e.g., WO 2010/033140, Cho et al. (2010) Lab Chip 10, 1567-1573; and Godin et al. (2008) J Biophoton. 1(5): 355-376. In both cases, cells can be labeled with multiple markers, allowing for the isolation of well-defined T cell subsets at high purity.

In some embodiments, the antibodies or binding partners are labeled with one or more detectable marker, to facilitate separation for positive and/or negative selection. For example, separation may be based on binding to fluorescently labeled antibodies. In some examples, separation of cells based on binding of antibodies or other binding partners specific for one or more cell surface markers are carried in a fluidic stream, such as by fluorescence-activated cell sorting (FACS), including preparative scale (FACS) and/or microelectromechanical systems (MEMS) chips, e.g., in combination with a flow-cytometric detection system. Such methods allow for positive and negative selection based on multiple markers simultaneously.

In some embodiments, the preparation methods include steps for freezing, e.g., cryopreserving, the cells, either before or after isolation, incubation, and/or engineering. In some embodiments, the freeze and subsequent thaw step removes granulocytes and, to some extent, monocytes in the cell population. In some embodiments, the cells are suspended in a freezing solution, e.g., following a washing step to remove plasma and platelets. Any of a variety of known freezing solutions and parameters in some aspects may be used. One example involves using PBS containing 20% DMSO and 8% human serum albumin (HSA), or other suitable cell freezing media. This is then diluted 1:1 with media so that the final concentration of DMSO and HSA are 10% and 4%, respectively. The cells are then frozen to −80 C at a rate of 1 C per minute and stored in the vapor phase of a liquid nitrogen storage tank.

In some embodiments, the provided methods include cultivation, incubation, culture, and/or genetic engineering steps. For example, in some embodiments, provided are methods for incubating and/or engineering the depleted cell populations and culture-initiating compositions.

Thus, in some embodiments, the cell populations are incubated in a culture-initiating composition. The incubation and/or engineering may be carried out in a culture vessel, such as a unit, chamber, well, column, tube, tubing set, valve, vial, culture dish, bag, or other container for culture or cultivating cells.

In some embodiments, the cells are incubated and/or cultured prior to or in connection with genetic engineering. The incubation steps can include culture, cultivation, stimulation, activation, and/or propagation. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor.

The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some embodiments, the stimulating conditions or agents include one or more agent, e.g., ligand, which is capable of stimulating or activating an intracellular signaling domain of a TCR complex. In some aspects, the agent turns on or initiates TCR/CD3 intracellular signaling cascade in a T cell. Such agents can include antibodies, such as those specific for a TCR component and/or costimulatory receptor, e.g., anti-CD3, anti-CD28, for example, bound to solid support such as a bead, and/or one or more cytokines. Optionally, the expansion method may further comprise the step of adding anti-CD3 and/or anti-CD28 antibody to the culture medium (e.g., at a concentration of at least about 0.5 ng/ml). In some embodiments, the stimulating agents include IL-2 and/or IL-15, for example, an IL-2 concentration of at least about 10 units/mL.

In some aspects, incubation is carried out in accordance with techniques such as those described in U.S. Pat. No. 6,040,177 to Riddell et al., Klebanoff et al. (2012) J Immunother. 35(9): 651-660, Terakura et al. (2012) Blood. 1:72-82, and/or Wang et al. (2012) J Immunother. 35(9): 689-701.

In some embodiments, the T cells are expanded by adding to the culture-initiating composition feeder cells, such as non-dividing peripheral blood mononuclear cells (PBMC), (e.g., such that the resulting population of cells contains at least about 5, 10, 20, or 40 or more PBMC feeder cells for each T lymphocyte in the initial population to be expanded); and incubating the culture (e.g. for a time sufficient to expand the numbers of T cells). In some aspects, the non-dividing feeder cells can comprise gamma-irradiated PBMC feeder cells. In some embodiments, the PBMC are irradiated with gamma rays in the range of about 3000 to 3600 rads to prevent cell division. In some aspects, the feeder cells are added to culture medium prior to the addition of the populations of T cells.

In some embodiments, the stimulating conditions include temperature suitable for the growth of human T lymphocytes, for example, at least about 25 degrees Celsius, generally at least about 30 degrees, and generally at or about 37 degrees Celsius. Optionally, the incubation may further comprise adding non-dividing EBV-transformed lymphoblastoid cells (LCL) as feeder cells. LCL can be irradiated with gamma rays in the range of about 6000 to 10,000 rads. The LCL feeder cells in some aspects is provided in any suitable amount, such as a ratio of LCL feeder cells to initial T lymphocytes of at least about 10:1.

In embodiments, antigen-specific T cells, such as antigen-specific CD4+ and/or CD8+ T cells, are obtained by stimulating naive or antigen specific T lymphocytes with antigen. For example, antigen-specific T cell lines or clones can be generated to cytomegalovirus antigens by isolating T cells from infected subjects and stimulating the cells in vitro with the same antigen.

### C. Engineered Cells, Vectors, and Compositions for Multi-Targeting

Also provided are cells such as engineered cells that can bind to multiple antigens. In some embodiments, improved selectivity and specificity is achieved through strategies targeting multiple antigens. Such strategies generally involve multiple antigen-binding domains, which typically are present on distinct genetically engineered antigen receptors and specifically bind to distinct antigens. In some embodiments, the cells are engineered with the ability to bind more than one antigen. For example, in some embodiments, the cells are engineered to express multispecific binding molecules. In some embodiments, the cells express multiple binding molecules, antigen-binding domains, each of which can target one antigen or multiple antigens, e.g., one binding domain that targets BAFF-R, such as any described herein, and another binding domain that targets another antigen, e.g., CD19.

In some aspects, the antigen receptor comprises a plurality of binding domains, which bind to different antigens, each expressed in or on the disease or condition to be targeted with the cells or tissues or cells thereof. Such features can in some aspects address or reduce the likelihood of off-target effects and/or increase response. For example, where a single antigen expressed in a disease or condition is also expressed on or in non-diseased or normal cells, such multi-targeting approaches can provide selectivity for desired cell types by requiring binding via multiple antigen receptors in order to activate the cell or induce a particular effector function. In some embodiments, a plurality of cells can be engineered to express a recombinant receptor (e.g., a CAR) comprising one or more different binding domains, e.g., a BAFF-R-binding domain and a CD19-binding domain.

Also provided are multispecific cells or compositions, such as those containing one or more of any of the recombinant receptors or cells provided herein. In some aspects, the multispecific cells such as cells containing a cell surface protein including the BAFF-R-binding domain or portion thereof and the CD19-binding domain or a portion thereof. In some embodiments, provided are compositions of cells that express recombinant receptors, wherein one or more of the binding domains binds and/or targets BAFF-R. In some embodiments, provided are compositions of cells that express recombinant receptors, wherein one or more of the binding domains binds and/or targets CD19. In some embodiments, the bispecific recombinant receptors or cells or compositions expressing the same target one or more epitopes on BAFF-R and one or more epitopes on CD19.

In some embodiments, provided are composition of cells, wherein cells within the composition expresses a bispecific recombinant receptor, e.g. a CAR. In some embodiments, the cell comprises (and in some cases has been transformed or transfected or transduced with) one or more vectors or constructs comprising one or more nucleic acid that encodes one or more amino acid sequence comprising one or more antibodies and/or portions thereof, e.g., antigen-binding fragments thereof. In some embodiments, one or more such cells are provided. In some embodiments, a composition containing one or more such cells is provided. In some embodiments, cells within the composition express a bispecific recombinant receptor, e.g., CAR. In some aspects, the provided embodiments include multi-targeting strategies that target BAFF-R and CD19.

In some embodiments, BAFF-R and CD19 are expressed or suspected of being expressed on the cell, tissue, or disease or condition being targeted, such as on the cancer cell. In some aspects, the cell, tissue, disease or condition is a BAFF-R-expressing cancer, a CD19-expressing cancer, or a BAFF-R- and CD19-expressing cancer.

### IV. PHARMACEUTICAL COMPOSITIONS

Also provided are compositions including the bispecific recombinant receptors (e.g. bispecific CARs targeting BAFF-R and CD19), and engineered cells, including pharmaceutical compositions and formulations. Among such compositions are those that include engineered cells, such as a plurality of engineered cells, expressing the provided bispecific recombinant receptors (e.g., CARs). In some embodiments, provided compositions include engineered cells, expressing the provided CARs that bind BAFF-R and CD19, such as those comprising a BAFF-R-binding domain and a CD19-binding domain.

Provided are pharmaceutical formulations comprising a bispecific recombinant receptor (CAR) that binds BAFF-R and CD19 and engineered cells expressing said receptors, a plurality of engineered cells expressing said receptors and/or additional agents for combination treatment or therapy. The pharmaceutical compositions and formulations generally include one or more optional pharmaceutically acceptable carrier(s) or excipient(s). In some embodiments, the composition includes at least one additional therapeutic agent.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some aspects, the choice of carrier is determined in part by the particular cell, binding molecule, and/or antibody, and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulation or composition may also contain more than one active ingredient useful for the particular indication, disease, or condition being treated with the binding molecules or cells, preferably those with activities complementary to the binding molecule or cell, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc. In some embodiments, the cells or antibodies are administered in the form of a salt, e.g., a pharmaceutically acceptable salt. Suitable pharmaceutically acceptable acid addition salts include those derived from mineral acids, such as hydrochloric, hydrobromic, phosphoric, metaphosphoric, nitric, and sulphuric acids, and organic acids, such as tartaric, acetic, citric, malic, lactic, fumaric, benzoic, glycolic, gluconic, succinic, and arylsulphonic acids, for example, p-toluenesulphonic acid.

Active ingredients may be entrapped in microcapsules, in colloidal drug delivery systems (for example, liposomes, albumin microspheres, microemulsions, nano-particles and nanocapsules) or in macroemulsions. In certain embodiments, the pharmaceutical composition is formulated as an inclusion complex, such as cyclodextrin inclusion complex, or as a liposome. Liposomes can serve to target the host cells (e.g., T-cells or NK cells) to a particular tissue. Many methods are available for preparing liposomes, such as those described in, for example, Szoka et al., Ann. Rev. Biophys. Bioeng., 9:467 (1980), and U.S. Pat. Nos. 4,235,871, 4,501,728, 4,837,028, and 5,019,369.

The pharmaceutical composition in some aspects can employ time-released, delayed release, and sustained release delivery systems such that the delivery of the composition occurs prior to, and with sufficient time to cause, sensitization of the site to be treated. Many types of release delivery systems are available and known. Such systems can avoid repeated administrations of the composition, thereby increasing convenience to the subject and the physician.

The pharmaceutical composition in some embodiments contains the binding molecules and/or cells in amounts effective to treat or prevent the disease or condition, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

In certain embodiments, in the context of genetically engineered cells containing the binding molecules, a subject is administered the range of at or about one million to at or about 100 billion cells, such as, e.g., at or about 1 million to at or about 50 billion cells (e.g., at or about 5 million cells, at or about 25 million cells, at or about 500 million cells, at or about 1 billion cells, at or about 5 billion cells, at or about 20 billion cells, at or about 30 billion cells, at or about 40 billion cells, or a range defined by any two of the foregoing values), such as at or about 10 million to at or about 100 billion cells (e.g., at or about 20 million cells, at or about 30 million cells, at or about 40 million cells, at or about 60 million cells, at or about 70 million cells, at or about 80 million cells, at or about 90 million cells, at or about 10 billion cells, at or about 25 billion cells, at or about 50 billion cells, at or about 75 billion cells, at or about 90 billion cells, or a range defined by any two of the foregoing values), and in some cases at or about 100 million cells to at or about 50 billion cells (e.g., at or about 120 million cells, at or about 250 million cells, at or about 350 million cells, at or about 450 million cells, at or about 650 million cells, at or about 800 million cells, at or about 900 million cells, at or about 3 billion cells, at or about 30 billion cells, at or about 45 billion cells) or any value in between these ranges, and/or such a number of cells per kilogram of body weight of the subject. In some aspects, in the context of genetically engineered cells expressing the binding molecules, e.g., CAR, a composition can contain at least the number of cells for administration for a dose of cell therapy, such as about or at least a number of cells described herein for administration.

The cells may be administered using standard administration techniques, formulations, and/or devices. Provided are formulations and devices, such as syringes and vials, for storage and administration of the compositions. Administration of the cells can be autologous or heterologous. For example, immune cells, immunoresponsive cells or progenitors can be obtained from one subject, and administered to the same subject or a different, compatible subject. Peripheral blood derived immunoresponsive cells or their progeny (e.g., in vivo, ex vivo or in vitro derived) can be administered via localized injection, including catheter administration, systemic injection, localized injection, intravenous injection, or parenteral administration. When administering a therapeutic composition (e.g., a pharmaceutical composition containing a genetically modified immune cell such as a T cell), it will generally be formulated in a unit dosage injectable form (solution, suspension, emulsion).

Formulations include those for oral, intravenous, intraperitoneal, subcutaneous, pulmonary, transdermal, intramuscular, intranasal, buccal, sublingual, or suppository administration. In some embodiments, the cell populations are administered parenterally. The term “parenteral,” as used herein, includes intravenous, intramuscular, subcutaneous, rectal, vaginal, intracranial, intrathoracic, and intraperitoneal administration. In some embodiments, the cell populations are administered to a subject using peripheral systemic delivery by intravenous, intraperitoneal, or subcutaneous injection.

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the binding molecule in a solvent, such as in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like.

Prolonged absorption of the injectable pharmaceutical form can be brought about by the use of agents delaying absorption, for example, aluminum monostearate and gelatin.

Sustained-release preparations may be prepared. Suitable examples of sustained-release preparations include semipermeable matrices of solid hydrophobic polymers containing the antibody, which matrices are in the form of shaped articles, e.g. films, or microcapsules.

The formulations to be used for in vivo administration are generally sterile. Sterility may be readily accomplished, e.g., by filtration through sterile filtration membranes.

Also provided are pharmaceutical compositions for combination therapy. Any of the additional agents for combination therapy described herein can be prepared and administered as one or more pharmaceutical compositions with the bispecific tandem CAR and/or engineered cells expressing said CAR described herein. The combination therapy can be administered in one or more pharmaceutical compositions, e.g., where the CARs and/or cells are in the same pharmaceutical composition as the additional agent, or in separate pharmaceutical compositions. For example, in some embodiments, the additional agent is an additional engineered cell, e.g., cell engineered to express a different CAR that targets a different epitope on PR3, and is administered in the same composition or in a separate composition. In some embodiments, each of the pharmaceutical composition is formulated in a suitable formulation according to the particular binding molecule, recombinant receptor, cell, e.g., engineered cell, and/or additional agent, and the particular dosage regimen and/or method of delivery.

### V. METHODS AND USES

Also provided are methods of using and uses of the BAFF-R- and CD19-targeted recombinant receptors, engineered cells, and pharmaceutical compositions and formulations thereof, such as in the treatment of diseases, conditions, and disorders in which BAFF-R and/or CD19 is expressed, or detection, diagnostic, and prognostic methods. Among such methods and uses are those that involve administering to a subject engineered cells, such as a plurality of engineered cells, expressing the provided bispecific recombinant receptors (e.g., CARs). Also provided are methods of combination therapy and/or treatment.

### A. Therapeutic and Prophylactic Methods and Uses

Also provided are methods of administering and uses of, such as therapeutic and prophylactic uses of, the bispecific recombinant receptors (e.g., CARs), engineered cells expressing the recombinant receptors (e.g., CARs), plurality of engineered cells expressing the receptors, and/or compositions comprising the same. Such methods and uses include therapeutic methods and uses, for example, involving administration of the molecules (e.g., recombinant receptors), cells (e.g., engineered cells), or compositions containing the same, to a subject having a disease, condition, or disorder associated with BAFF-R and/or CD19 such as a disease, condition, or disorder associated with BAFF-R and/or CD19 expression, and/or in which cells or tissues express, e.g., specifically express, BAFF-R and/or CD19. Such methods and uses include therapeutic methods and uses, for example, involving administration of the molecules (e.g., recombinant receptors), cells (e.g., engineered cells), or compositions containing the same, to a subject having a disease, condition, or disorder associated with BAFF-R such as a disease, condition, or disorder associated with BAFF-R expression, and/or in which cells or tissues express, e.g., specifically express, BAFF-R. Such methods and uses include therapeutic methods and uses, for example, involving administration of the molecules (e.g., recombinant receptors), cells (e.g., engineered cells), or compositions containing the same, to a subject having a disease, condition, or disorder associated with CD19 such as a disease, condition, or disorder associated with CD19 expression, and/or in which cells or tissues express, e.g., specifically express, CD19. Such methods and uses include therapeutic methods and uses, for example, involving administration of the molecules (e.g., recombinant receptors), cells (e.g., engineered cells), or compositions containing the same, to a subject having a disease, condition, or disorder associated with BAFF-R and CD19 such as a disease, condition, or disorder associated with BAFF-R and CD19 expression, and/or in which cells or tissues express, e.g., specifically express, BAFF-R and CD19.

In some embodiments, the molecule, cell, and/or composition is/are administered in an effective amount to effect treatment of the disease or disorder. Provided herein are uses of the recombinant receptors (e.g., CARs), and cells (e.g., engineered cells) in such methods and treatments, and in the preparation of a medicament in order to carry out such therapeutic methods. In some embodiments, the methods are carried out by administering the binding molecules or cells, or compositions comprising the same, to the subject having, having had, or suspected of having the disease or condition. In some embodiments, the methods thereby treat the disease or condition or disorder in the subject. Also provided herein are use of any of the compositions, such as pharmaceutical compositions provided herein, for the treatment of a disease or disorder associated with BAFF-R and/or CD19, such as use in a treatment regimen. Also provided herein are use of any of the compositions, such as pharmaceutical compositions provided herein, for the treatment of a disease or disorder associated with BAFF-R, such as use in a treatment regimen. Also provided herein are use of any of the compositions, such as pharmaceutical compositions provided herein, for the treatment of a disease or disorder associated with CD19, such as use in a treatment regimen. Also provided herein are use of any of the compositions, such as pharmaceutical compositions provided herein, for the treatment of a disease or disorder associated with BAFF-R and CD19, such as use in a treatment regimen.

As used herein, “treatment” (and grammatical variations thereof such as “treat” or “treating”) refers to complete or partial amelioration or reduction of a disease or condition or disorder, or a symptom, adverse effect or outcome, or phenotype associated therewith. Desirable effects of treatment include, but are not limited to, preventing recurrence of disease, alleviation of symptoms, diminishment of any direct or indirect pathological consequences of the disease, preventing metastasis, decreasing the rate of disease progression, amelioration or palliation of the disease state, and remission or improved prognosis. The terms do not imply complete curing of a disease or complete elimination of any symptom or effect(s) on all symptoms or outcomes.

As used herein, “delaying development of a disease” means to defer, hinder, slow, retard, stabilize, suppress and/or postpone development of the disease (such as cancer). This delay can be of varying lengths of time, depending on the history of the disease and/or subject being treated. As sufficient or significant delay can, in effect, encompass prevention, in that the subject does not develop the disease. For example, a late stage cancer, such as development of metastasis, may be delayed.

“Preventing,” as used herein, includes providing prophylaxis with respect to the occurrence or recurrence of a disease in a subject that may be predisposed to the disease but has not yet been diagnosed with the disease. In some embodiments, the provided molecules and compositions are used to delay development of a disease or to slow the progression of a disease.

As used herein, to “suppress” a function or activity is to reduce the function or activity when compared to otherwise same conditions except for a condition or parameter of interest, or alternatively, as compared to another condition. For example, an antibody or composition or cell which suppresses tumor growth reduces the rate of growth of the tumor compared to the rate of growth of the tumor in the absence of the antibody or composition or cell.

An “effective amount” of an agent, e.g., a pharmaceutical formulation, binding molecule, antibody, cells, or composition, in the context of administration, refers to an amount effective, at dosages/amounts and for periods of time necessary, to achieve a desired result, such as a therapeutic or prophylactic result.

A “therapeutically effective amount” of an agent, e.g., a pharmaceutical formulation, binding molecule, antibody, cells, or composition refers to an amount effective, at dosages and for periods of time necessary, to achieve a desired therapeutic result, such as for treatment of a disease, condition, or disorder, and/or pharmacokinetic or pharmacodynamic effect of the treatment. The therapeutically effective amount may vary according to factors such as the disease state, age, sex, and weight of the subject, and the populations of cells administered. In some embodiments, the provided methods involve administering the molecules, antibodies, cells, and/or compositions at effective amounts, e.g., therapeutically effective amounts.

A “prophylactically effective amount” refers to an amount effective, at dosages and for periods of time necessary, to achieve the desired prophylactic result. Typically, but not necessarily, since a prophylactic dose is used in subjects prior to or at an earlier stage of disease, the prophylactically effective amount will be less than the therapeutically effective amount.

As used herein, a “subject” or an “individual” is a mammal. In some embodiments, a “mammal” includes humans, non-human primates, domestic and farm animals, and zoo, sports, or pet animals, such as dogs, horses, rabbits, cattle, pigs, hamsters, gerbils, mice, ferrets, rats, cats, monkeys, etc. In some embodiments, the subject is human.

The disease or condition that is treated can be any in which expression of an antigen is associated with and/or involved in the etiology of a disease condition or disorder, e.g. causes, exacerbates or otherwise is involved in such disease, condition, or disorder. Exemplary diseases and conditions can include diseases or conditions associated with malignancy or transformation of cells (e.g. cancer), autoimmune or inflammatory disease, or an infectious disease, e.g. caused by a bacterial, viral or other pathogen. Exemplary antigens, which include antigens associated with various diseases and conditions that can be treated, are described above. In particular embodiments, the chimeric antigen receptor (CAR) or transgenic TCR specifically binds to an antigen associated with the disease or condition.

Among the diseases, conditions, and disorders are tumors, including solid tumors, hematologic malignancies, and melanomas, and including localized and metastatic tumors, infectious diseases, such as infection with a virus or other pathogen, e.g., HIV, HCV, HBV, CMV, HPV, and parasitic disease, and autoimmune and inflammatory diseases.

Provided herein are methods of treatment that involve administering engineered cells or compositions containing engineered cells, such as engineered T cells, including methods for the treatment of subjects with systemic autoimmune diseases. In some embodiments, provided herein are methods and use of BAFFR/CD19-directed CAR engineered cells (e.g., T cells) and/or compositions thereof, including methods for the treatment of subjects with systemic autoimmune diseases. In some embodiments, the systemic autoimmune disease is a severe and/or moderate systemic autoimmune diseases that have failed at least two or more prior therapies. In particular embodiments, the method includes administering to the subject a dose of T cells that includes CD4+ and CD8+ T cells, wherein the T cells comprises a chimeric antigen receptor (CAR) that specifically binds to BAFFR and CD19. In particular embodiments, the method includes administering to the subject a dose of T cells that includes CD4+ and CD8+ T cells, wherein the T cells comprises a chimeric antigen receptor (CAR) that specifically binds to BAFFR and CD19.

In some embodiment, the methods provided herein are used to treat autoimmune diseases caused by, associated with and/or specific to cells expressing CD19 and/or BAFFR. In some embodiment, the methods provided herein are used to treat autoimmune diseases caused by, associated with and/or specific to cells expressing CD19 and BAFFR.

In some embodiments, the immune disease include, but are not limited to, Addison's disease, allergies, ankylosing spondylitis, asthma, atherosclerosis, autoimmune diseases of the ear, autoimmune diseases of the eye, autoimmune hepatitis, autoimmune parotitis, colitis, coronary heart disease, diabetes, including Type 1 and/or Type 2 diabetes, epididymitis, glomerulonephritis, Graves' disease, Guillain-Barre syndrome, Hashimoto's disease, hemolytic anemia, idiopathic thrombocytopeniarpura, inflammatory bowel disease, immune response to recombinant drug products, myasthenia gravis, pemphigus, psoriasis, rheumatic fever, rheumatoid arthritis, sarcoidosis, scleroderma, spondyloarthropathies, thyroiditis, transplant rejection, vasculitis, AIDS, atopic allergy, bronchial asthma, eczema, leprosy, schizophrenia, chronic fatigue syndrome, Alzheimer's disease, Parkinson's disease, myocardial infarction, stroke, autism, epilepsy, Arthus's phenomenon, and anaphylaxis. In some embodiments, the systemic autoimmune diseases comprises, systemic lupus erythematosus (SLE) and severe SLE, rheumatoid arthritis (RA), and systemic sclerosis (SSc). In some embodiments, the systemic autoimmune diseases may include Systemic Lupus Erythematosus (SLE), Sjogren's' syndrome, progressive systemic sclerosis (i.e., scleroderma), idiopathic inflammatory myositis (IIM, including dermatomyositis, polymyositis and necrotizing myositis), mixed connective tissue disorder (MCTD), relapsing-remitting multiple sclerosis, ANCA-associated vasculitis (AAV), Crohn's disease, myasthenia gravis, Behçet's, rheumatoid arthritis, multiple sclerosis, primary progressive MS, IgA nephropathy, pemphigus vulgaris, myasthernia gravis, autoimmune hemolytic anemia, immune thrombocytopenia, IgG4-related diseases, membranous nephropathy, cutaneous lupus erythematosus, sarcoidosis, light chain amyloidosis, acute respiratory distress syndrome, atopic eczema, hereditary angioedema, hidradenitis suppurative, inclusion-body myositis, inflammatory bowel disease, mastocytosis, multifocal motor neuropathy, necrotizing myopathy, neuromyelitis optica spectrum disorder, mixed connective tissue disorder, POEMS syndrome, primary biliary cholangitis, psoriasis, rhesus hemolytic disease, Still's disease, type 1 diabetes, urticaria, capillary leakage syndrome, cytokine release syndrome, erythema multiforme, pyoderma gangrenosum, x-linked agammaglobulinemia, antiphospholipid syndrome, and chronic inflammatory demyelinating polyneuropathy. and chronic inflammatory demyelinating polyneuropathy.

In some embodiment, the methods provided herein are used to treat autoimmune diseases such as, SLE, IIM, SSc, AAV, systemic sclerosis, multiple sclerosis (MS), highly active relapsing remitting MS, primary progressive MS, IgA nephropathy, pemphigus vulgaris, myasthernia gravis, demyelinating polyradiculoneuropathy, autoimmune hemolytic anemia, immune thrombocytopenia, IgG4-related diseases, membranous nephropathy, Primary Sjorgren's syndrome, cutaneous lupus erythematosus, sarcoidosis, light chain amyloidosis, rheumatoid arthritis, bullous pemphigoid, acute respiratory distress syndrome, atopic eczema, hereditary angioedema, hidradenitis suppurative, inclusion-body myositis, inflammatory bowel disease, mastocytosis, multifocal motor neuropathy, necrotizing myopathy, neuromyelitis optica spectrum disorder, mixed connective tissue disorder, POEMS syndrome, primary biliary cholangitis, psoriasis, rhesus hemolytic disease, Still's disease, type 1 diabetes, urticaria, capillary leakage syndrome, cytokine release syndrome, erythema multiforme, pyoderma gangrenosum, antiphospholipid syndrome, or x-linked agammaglobulinemia. In some embodiments, the methods provided herein are used to treat of SLE, IMM, AAV, systemic sclerosis, highly active relapsing remitting MS, primary progressive MS, IgA nephropathy, pemphigus vulgaris, or myasthernia gravis. In some embodiments, the methods provided herein are used to treated SLE. In some embodiments, the methods provided herein are used to treated IIM. In some embodiments, the methods provided herein are used to treated SSc. In some embodiments, the methods provided herein are used to treat MS.

In some embodiments, the systemic autoimmune disease is SLE, such as a moderate SLE or severe refractory SLE, idiopathic inflammatory myopathy, systemic sclerosis, rheumatoid arthritis (RA), or multiple sclerosis. Among provided methods are methods of treatment that involve administering engineered cells or compositions containing engineered cells, such as engineered T cells to subjects with SLE, including severe refractory SLE. Also provided are methods and uses of provided BAFFR/CD19-directed CAR engineered cells (e.g., T cells) and/or compositions thereof, including methods for the treatment of subjects having a SLE, including severe refractory SLE, that involves administration of the engineered cells and/or compositions thereof. In certain embodiments, the subject has severe refractory SLE. In some embodiments, the subject is selected for or identified as having severe refractory SLE, such as by the presence of certain features or clinical manifestations that indicate the presence of severe refractory SLE. In some embodiments, the methods and use of provided BAFFR/CD19-directed CAR engineered cells (e.g., T cells) and/or compositions thereof, include methods for the treatment of subjects with severe refractory SLE that have failed at least two or more prior therapies. In particular embodiments, the method includes administering to the subject a dose of T cells that includes CD4+ and CD8+ T cells, wherein the T cells comprises a chimeric antigen receptor (CAR) that specifically binds to BAFFR and CD19.

In some embodiments, the disease, disorder or condition to be treated is a tumor, cancer, malignancy, neoplasm, or other proliferative disease or disorder. In some embodiments, the disease or condition is a B cell malignancy. Such diseases include but are not limited to leukemia, lymphoma, and multiple myeloma (MM). In some embodiments, the B cell malignancy is selected from among acute lymphoblastic leukemia (ALL), adult ALL, pro-lymphocytic leukemias, hairy cell leukemias, small lymphocytic lymphoma (SLL), common acute lymphocytic leukemias, chronic lymphoblastic leukemia (CLL), Null-acute lymphoblastic leukemias, follicular lymphoma, splenic lymphoma, marginal zone lymphoma, mantle cell lymphoma, indolent B cell lymphoma, Anaplastic large cell lymphoma (ALCL), Hodgkin lymphoma, non-Hodgkin lymphoma (NHL), and Diffuse Large B-Cell Lymphoma (DLBCL). In some embodiments, the disease or condition is NHL. In some embodiments, the NHL is selected from the group consisting of aggressive NHL, diffuse large B cell lymphoma (DLBCL), NOS (de novo and transformed from indolent), primary mediastinal large B cell lymphoma (PMBCL), T cell/histocyte-rich large B cell lymphoma (TCHRBCL), Burkitt's lymphoma, mantle cell lymphoma (MCL), and/or follicular lymphoma (FL), or refractory follicular lymphoma, optionally, follicular lymphoma Grade 3B (FL3B).

In some embodiments, the disease or disorder is a B cell-related disorder. In some of any of the provided embodiments of the provided methods, the disease or disorder is an autoimmune disease or disorder. In some of any of the provided embodiments of the provided methods, the autoimmune disease or disorder is systemic lupus erythematosus (SLE), lupus nephritis, inflammatory bowel disease, rheumatoid arthritis, ANCA associated vasculitis, idiopathic thrombocytopenia purpura (ITP), thrombotic thrombocytopenia purpura (TTP), autoimmune thrombocytopenia, Chagas' disease, Grave's disease, Wegener's granulomatosis, poly-arteritis nodosa, Sjogren's syndrome, pemphigus vulgaris, scleroderma, Crohn's disease, asthma, multiple sclerosis, psoriasis, IgA nephropathy, IgM polyneuropathies, vasculitis, diabetes mellitus, Reynaud's syndrome, anti-phospholipid syndrome, Goodpasture's disease, Kawasaki disease, autoimmune hemolytic anemia, myasthenia gravis, progressive glomerulonephritis, and/or a disease or condition associated with transplant.

Methods for administration of cells for adoptive cell therapy are known and may be used in connection with the provided methods and compositions. For example, adoptive T cell therapy methods are described, e.g., in US Pat. App. Pub. No. 2003/0170238 to Gruenberg et al; U.S. Pat. No. 4,690,915 to Rosenberg; Rosenberg (2011) Nat Rev Clin Oncol. 8(10): 577-85). See, e.g., Themeli et al. (2013) Nat Biotechnol. 31(10): 928-933; Tsukahara et al. (2013) Biochem Biophys Res Commun 438(1): 84-9; Davila et al. (2013) PLOS ONE 8(4): e61338.

Among the diseases to be treated is any disease or disorder associated with BAFF-R and/or CD19 or any disease or disorder in which BAFF-R and/or CD19 is specifically expressed and/or in which BAFF-R and/or CD19 has been targeted for treatment (also referred to herein interchangeably as a “BAFF-R-associated disease or disorder” or a “CD19-associated disease or disorder”). In some embodiments, the disease or disorder associated with BAFF-R and/or CD19 is cancer.

In some embodiments, the disease or disorder is associated with expression of BAFF-R and CD19. In some embodiments, cells of the disease are suspected of expressing both antigens. In some embodiments, one or both of the antigens is susceptible to antigen loss, in that some cells of the disease may no longer express both antigens. Thus, in some embodiments, a dual-targeting approach, targeting both BAFF-R and CD19, may be advantageous.

In some embodiments, the methods may identify a subject who has, is suspected to have, or is at risk for developing a BAFF-R-associated and/or CD19-associated disease or disorder. Hence, provided are methods for identifying subjects with diseases or disorders associated with BAFF-R and/or CD19 expression and selecting them for treatment with a provided bispecific recombinant receptors (e.g., CARs), and/or engineered cells expressing the recombinant receptors.

For example, a subject may be screened for the presence of a disease or disorder associated with elevated BAFF-R and/or CD19 expression, such as a BAFF-R- and/or CD19-expressing cancer. In some embodiments, the methods include screening for or detecting the presence of a BAFF-R- and/or CD19-associated disease, e.g., a tumor. Thus, in some aspects, a sample may be obtained from a patient suspected of having a disease or disorder associated with elevated BAFF-R and/or CD19 expression and assayed for the expression level of BAFF-R and/or CD19. In some aspects, a subject who tests positive for a BAFF-R- and/or CD19-associated disease or disorder may be selected for treatment by the present methods, and may be administered a therapeutically effective amount of a composition comprising cells expressing a recombinant receptor (e.g., CAR) comprising a BAFF-R-binding domain and a CD19-binding domain, or a pharmaceutical composition thereof as described herein. In some aspects, a subject who tests positive for a BAFF-R- and/or CD19-associated disease or disorder may be selected for treatment by the present methods, and may be administered a therapeutically effective amount of a composition comprising cells expressing a recombinant receptor (e.g., CAR) comprising a BAFF-R-binding domain and a CD19-binding domain, cells expressing a recombinant receptor comprising a BAFF-R-binding domain and/or a CD19-binding domain, or a pharmaceutical composition thereof as described herein.

In some embodiments, the subject has received a prior therapy that is a BAFF-R CAR therapy or other BAFF-R-targeted therapy. In some embodiments, the subject is refractory to or has relapsed following such BAFF-R CAR therapy or other BAFF-R-targeted therapy.

In some embodiments, the subject has persistent or relapsed disease, e.g., following treatment with a BAFF-R-specific antibody and/or cells expressing a BAFF-R-targeting chimeric receptor and/or other therapy, including chemotherapy, radiation, and/or hematopoietic stem cell transplantation (HSCT), e.g., allogenic HSCT or autologous HSCT. In some embodiments, the administration effectively treats the subject despite the subject having become resistant to another BAFF-R-targeted therapy. In some embodiments, the subject has persistent or relapsed disease, e.g., following treatment with a CD19-specific antibody and/or cells expressing a CD19-targeting chimeric receptor and/or other therapy, including chemotherapy, radiation, and/or hematopoietic stem cell transplantation (HSCT), e.g., allogenic HSCT or autologous HSCT. In some embodiments, the administration effectively treats the subject despite the subject having become resistant to another CD19-targeted therapy. In some embodiments, the subject has not relapsed but is determined to be at risk for relapse, such as at a high risk of relapse, and thus the compound or composition is administered prophylactically, e.g., to reduce the likelihood of or prevent relapse.

In some embodiments, the subject has received a prior therapy that is a CD19 CAR therapy or other CD19-targeted therapy. In some embodiments, the subject is refractory to or has relapsed following such CD19 CAR therapy or other CD19-targeted therapy. In some cases, the subject is refractory to or has relapsed due to CD19 antigen-negative tumor cells and/or CD19 antigen/epitope loss following therapy.

In some embodiments, the subject has persistent or relapsed disease following treatment with another therapy, such as treatment with a CD19-specific antibody, CD19-targeting receptor, and/or cells expressing a CD19-targeting chimeric receptor. In some embodiments, the administration effectively treats the subject despite the subject having become resistant to another therapy, such as a CD19-targeted therapy. In some embodiments, the subject has not relapsed but is determined to be at risk for relapse, such as at a high risk of relapse, and thus the compound or composition is administered prophylactically, e.g., to reduce the likelihood of or prevent relapse.

In some embodiments, the subject is one that is eligible for a transplant, such as is eligible for a hematopoietic stem cell transplantation (HSCT), e.g., allogenic HSCT or autologous HSCT. In some of such embodiments, the subject has not previously received a transplant, despite being eligible, prior to administration of the bispecific recombinant receptors (e.g., CARs), engineered cells expressing the recombinant receptors (e.g., CARs), plurality of engineered cells expressing the receptors, and/or compositions comprising the same, as provided herein.

In some embodiments, the subject is one that is not eligible for a transplant, such as is not eligible for a hematopoietic stem cell transplantation (HSCT), e.g., allogenic HSCT or autologous HSCT. In some of such embodiments, such a subject is administered the bispecific recombinant receptors (e.g., CARs), engineered cells expressing the recombinant receptors (e.g., CARs), plurality of engineered cells expressing the receptors, and/or compositions comprising the same, according to the provided embodiments herein.

In some embodiments, prior to the initiation of administration of the engineered cells, the subject has received one or more prior therapies. In some embodiments, the subject has received at least 1, 2, 3, 4, 5, 6, 7, 8, 9, 10, 11, 12, 13, 14, 15, 16, 17, 18, 19 or 20 or more prior therapies. In some embodiments, the subject has received at least 3, 4, 5, 6, 7, 8, 9, 10 or more prior therapies.

In some aspects, the subject has relapsed following, or has been refractory to, one or more of, for example, each, individually, of the one or more prior therapies. In some aspects, the prior therapies include treatment with autologous stem cell transplant (ASCT); an immunomodulatory agent; a proteasome inhibitor; and an anti-CD38 antibody; unless the subject was not a candidate for or was contraindicated for one or more of the therapies. In some embodiments, the immunomodulatory agent is selected from among thalidomide, lenalidomide or pomalidomide. In some embodiments, the proteasome inhibitor is selected from among bortezomib, carfilzomib or ixazomib. In some embodiments, the anti-CD38 antibody is or comprises daratumumab. In some embodiments, the subject must have undergone at least 2 consecutive cycles of treatment for each regimen unless progressive disease was the best response to the regimen.

In some embodiments, the treatment does not induce an immune response by the subject to the therapy, and/or does not induce such a response to a degree that prevents effective treatment of the disease or condition. In some aspects, the degree of immunogenicity and/or graft versus host response is less than that observed with a different but comparable treatment. For example, in the case of adoptive cell therapy using cells expressing CARs including the provided CD19- and BAFF-R-binding domains, the degree of immunogenicity in some embodiments is reduced compared to CARs including a different antibody that binds to a similar, e.g., overlapping epitope and/or that competes for binding to BAFF-R or CD19 with the antibody, such as a mouse or monkey or rabbit or humanized antibody.

In some embodiments, the methods include adoptive cell therapy, whereby genetically engineered cells expressing the provided recombinant receptors comprising a BAFF-R-binding domain and a CD19-binding domain (e.g., CARs comprising anti-BAFF-R antibody or antigen-binding fragment thereof and anti-CD19 antibody or antigen-binding fragment thereof) are administered to subjects. Such administration can promote activation of the cells (e.g., T cell activation) in a BAFF-R- and/or CD19-targeted manner, such that the cells of the disease or disorder are targeted for destruction.

Thus, the provided methods and uses include methods and uses for adoptive cell therapy. In some embodiments, the methods include administration of the cells or a composition containing the cells to a subject, tissue, or cell, such as one having, at risk for, or suspected of having the disease, condition or disorder. In some embodiments, the cells, populations, and compositions are administered to a subject having the particular disease or condition to be treated, e.g., via adoptive cell therapy, such as adoptive T cell therapy. In some embodiments, the cells or compositions are administered to the subject, such as a subject having or at risk for the disease or condition. In some aspects, the methods thereby treat, e.g., ameliorate one or more symptom of the disease or condition, such as by lessening tumor burden in a BAFF-R- and/or CD19-expressing cancer. In some aspects, the methods thereby treat, e.g., ameliorate one or more symptom of the disease or condition, such as by lessening tumor burden in a BAFF-R-expressing cancer. In some aspects, the methods thereby treat, e.g., ameliorate one or more symptom of the disease or condition, such as by lessening tumor burden in a CD19-expressing cancer. In some aspects, the methods thereby treat, e.g., ameliorate one or more symptom of the disease or condition, such as by lessening tumor burden in a BAFF-R- and CD19-expressing cancer.

Methods for administration of cells for adoptive cell therapy are known and may be used in connection with the provided methods and compositions. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10): 577-85). See, e.g., Themeli et al. (2013) Nat Biotechnol. 31(10): 928-933; Tsukahara et al. (2013) Biochem Biophys Res Commun 438(1): 84-9; Davila et al. (2013) PLOS ONE 8(4): e61338.

In some embodiments, the cell therapy, e.g., adoptive cell therapy, e.g., adoptive T cell therapy, is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

In some embodiments, the subject, to whom the cells, cell populations, or compositions are administered is a primate, such as a human. In some embodiments, the primate is a monkey or an ape. The subject can be male or female and can be any suitable age, including infant, juvenile, adolescent, adult, and geriatric subjects. In some embodiments, the subject is a non-primate mammal, such as a rodent. In some examples, the patient or subject is a validated animal model for disease, adoptive cell therapy, and/or for assessing toxic outcomes such as cytokine release syndrome (CRS).

The bispecific recombinant receptors (e.g., CARs) and cells expressing the same, can be administered by any suitable means, for example, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. The bispecific recombinant receptors (e.g., CARs) and cells expressing the same can be administered by any suitable means, for example, by injection, e.g., intravenous or subcutaneous injections, intraocular injection, periocular injection, subretinal injection, intravitreal injection, trans-septal injection, subscleral injection, intrachoroidal injection, intracameral injection, subconjunctival injection, subconjunctival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, intracranial, intrathoracic, or subcutaneous administration. Dosing and administration may depend in part on whether the administration is brief or chronic. Various dosing schedules include but are not limited to single or multiple administrations over various time-points, bolus administration, and pulse infusion.

For the prevention or treatment of disease, the appropriate dosage of the binding molecule or cell may depend on the type of disease to be treated, the type of binding molecule, the severity and course of the disease, whether the binding molecule is administered for preventive or therapeutic purposes, previous therapy, the patient's clinical history and response to the binding molecule, and the discretion of the attending physician.

The compositions and molecules and cells are in some embodiments suitably administered to the patient at one time or over a series of treatments.

For the prevention or treatment of disease, the appropriate dosage of the binding molecule, recombinant receptor or cell may depend on the type of disease to be treated, the type of binding molecule or recombinant receptor, the severity and course of the disease, whether the binding molecule or recombinant receptor is administered for preventive or therapeutic purposes, previous therapy, the patient's clinical history and response to the recombinant receptor or cell, and the discretion of the attending physician. The compositions and molecules and cells are in some embodiments suitably administered to the patient at one time or over a series of treatments.

In some embodiments, the dose and/or frequency of administration is/are determined based on efficacy and/or response. In some embodiments, efficacy is determined by evaluating disease status. Exemplary methods for assessing disease status include: measurement of M protein in biological fluids, such as blood and/or urine, by electrophoresis and immunofixation; quantification of sFLC (κ and λ) in blood; skeletal survey; and imaging by positron emission tomography (PET)/computed tomography (CT) in subjects with extramedullary disease. In some embodiments, disease status can be evaluated by bone marrow examination.

In some examples, dose and/or frequency of administration is determined by the expansion and persistence of the recombinant receptor or cell in the blood and/or bone marrow. In some embodiments, dose and/or frequency of administration is determined based on the antitumor activity of the recombinant receptor or engineered cell. In some embodiments antitumor activity is determined by the overall response rate (ORR) and/or International Myeloma Working Group (IMWG) Uniform Response Criteria (see Kumar et al. (2016) Lancet Oncol 17(8): e328-346). In some embodiments, response is evaluated using minimal residual disease (MRD) assessment. In some embodiments, MRD can be assessed by methods such as flow cytometry and high-throughput sequencing, e.g., deep sequencing. In some embodiments, response is evaluated based on the duration of response following administration of the recombinant receptor or cells. In some examples, dose and/or frequency of administration can be based on toxicity. In some embodiments, dose and/or frequency can be determined based on health-related quality of life (HRQOL) of the subject to which the recombinant receptor and/or cells is/are administered. In some embodiments, dose and/or frequency of administration can be changed, i.e., increased or decreased, based on any of the above criteria.

In some embodiments, the Eastern Cooperative Oncology Group (ECOG) performance status indicator can be used to assess or select subjects for treatment, e.g., subjects who have had poor performance from prior therapies (see, e.g., Oken et al. (1982) Am J Clin Oncol. 5:649-655). The ECOG Scale of Performance Status describes a patient's level of functioning in terms of their ability to care for themselves, daily activity, and physical ability (e.g., walking, working, etc.). In some embodiments, an ECOG performance status of 0 indicates that a subject can perform normal activity. In some aspects, subjects with an ECOG performance status of 1 exhibit some restriction in physical activity but the subject is fully ambulatory. In some aspects, patients with an ECOG performance status of 2 is more than 50% ambulatory. In some cases, the subject with an ECOG performance status of 2 may also be capable of selfcare; see e.g., Sørensen et al., (1993) Br J Cancer 67(4) 773-775. In some embodiments, the subject that are to be administered according to the methods or treatment regimen provided herein include those with an ECOG performance status of 0 or 1.

In some embodiments, the administration can treat the subject despite the subject having become resistant to another therapy. In some embodiments, when administered to subjects according to the embodiments described herein, the dose or the composition is capable of achieving objective response (OR), in at least 50%, 60%, 70%, 80%, 90%, or 95% of subjects that were administered. In some embodiments, OR includes subjects who achieve stringent complete response (sCR), complete response (CR), very good partial response (VGPR), partial response (PR) and minimal response (MR). In some embodiments, when administered to subjects according to the embodiments described herein, the dose or the composition is capable of achieving stringent complete response (sCR), complete response (CR), very good partial response (VGPR) or partial response (PR), in at least 50%, 60%, 70%, 80%, or 85% of subjects that were administered. In some embodiments, when administered to subjects according to the embodiments described herein, the dose or the composition is capable of achieving stringent complete response (sCR) or complete response (CR) at least 20%, 30%, 40% 50%, 60% or 70% of subjects that were administered. In some embodiments, exemplary doses include about 5.0×10⁷, 1.5×10⁸, 3.0×10⁸ or 4.5×10⁸ CAR-expressing T cells. In some embodiments, exemplary doses include about 1.0×10⁷, 1.25×10⁷, 1.5×10⁷, 1.75×10⁷, 2.0×10⁷, 2.5×10⁷, 3.0×10⁷, 3.5×10⁷, 4.0×10⁷, 4.5×10⁷, 5.0×10⁷, 7.5×10⁷, 1.5×10⁸, 2.25×10⁸, 3.0×10⁸, 4.5×10⁸, or 6.0×10⁸ CAR-expressing T cells. In some embodiments, the dose comprises about 1.0×10⁷ CAR-expressing T cells. In some embodiments, the dose comprises about 1.25×10⁷ CAR-expressing T cells. In some embodiments, the dose comprises about 1.5×10⁷ CAR-expressing T cells. In some embodiments, the dose comprises about 1.75×10⁷ CAR-expressing T cells. In some embodiments, the dose comprises about 2.0×10⁷ CAR-expressing T cells. In some embodiments, the dose comprises about 2.5×10⁷ CAR-expressing T cells. In some embodiments, the dose comprises about 3.0×10⁷ CAR-expressing T cells. In some embodiments, the dose comprises about 3.5×10⁷ CAR-expressing T cells. In some embodiments, the dose comprises about 4.0×10⁷ CAR-expressing T cells. In some embodiments, the dose comprises about 4.5×10⁷ CAR-expressing T cells. In some embodiments, the dose comprises about 5.0×10⁷ CAR-expressing T cells. In some embodiments, the dose comprises about 6.0×10⁷ CAR-expressing T cells. In some embodiments, the dose comprises about 7.0×10⁷ CAR-expressing T cells. In some embodiments, the dose comprises about 7.5×10⁷. In some embodiments, the dose comprises about 8.0×10⁷ CAR-expressing T cells. In some embodiments, the dose comprises about 9.0×10⁷ CAR-expressing T cells. In some embodiments, the dose comprises about 1.0×10⁸ CAR-expressing T cells. In some embodiments, the dose comprises about 1.25×10⁸ CAR-expressing T cells. In some embodiments, the dose comprises about 1.5×10⁸ CAR-expressing T cells. In some embodiments, the dose comprises about 1.75×10⁸ CAR-expressing T cells. In some embodiments, the dose comprises about 2.0×10⁸ CAR-expressing T cells. In some embodiments, the dose comprises about 2.25×10⁸ CAR-expressing T cells. In some embodiments, the dose comprises about 2.5×10⁸ CAR-expressing T cells. In some embodiments, the dose comprises about 3.0×10⁸ CAR-expressing T cells. In some embodiments, the dose comprises about 3.5×10⁸ CAR-expressing T cells. In some embodiments, the dose comprises about 4.0×10⁸ CAR-expressing T cells. In some embodiments, the dose comprises about 4.5×10⁸ CAR-expressing T cells. In some embodiments, the dose comprises about 6.0×10⁸ CAR-expressing T cells. In some aspects, particular response to the treatment, e.g., according to the methods provided herein, can be assessed based on the International Myeloma Working Group (IMWG) Uniform Response Criteria (see Kumar et al. (2016) Lancet Oncol 17(8): e328-346). In some embodiments, exemplary doses to achieve particular outcomes, such as OR, includes about 1.0×10⁷ CAR-expressing T cells. In some embodiments, exemplary doses to achieve particular outcomes, such as OR, includes about 5.0×10⁷ CAR-expressing T cells. In some embodiments, exemplary doses to achieve particular outcomes, such as OR, includes about 1.0×10⁸ CAR-expressing T cells. In some embodiments, exemplary doses to achieve particular outcomes, such as OR, includes about 1.5×10⁸ CAR-expressing T cells.

In some embodiments, toxicity and/or side-effects of treatment can be monitored and used to adjust dose and/or frequency of administration of the recombinant receptor, e.g., CAR, cells, and or compositions. For example, adverse events and laboratory abnormalities can be monitored and used to adjust dose and/or frequency of administration. Adverse events include infusion reactions, cytokine release syndrome (CRS), neurotoxicity, macrophage activation syndrome, and tumor lysis syndrome (TLS). Any of such events can establish dose-limiting toxicities and warrant decrease in dose and/or a termination of treatment. Other side effects or adverse events which can be used as a guideline for establishing dose and/or frequency of administration include non-hematologic adverse events, which include but are not limited to fatigue, fever or febrile neutropenia, increase in transaminases for a set duration (e.g., less than or equal to 2 weeks or less than or equal to 7 days), headache, bone pain, hypotension, hypoxia, chills, diarrhea, nausea/vomiting, neurotoxicity (e.g., confusion, aphasia, seizures, convulsions, lethargy, and/or altered mental status), disseminated intravascular coagulation, other asymptomatic non-hematological clinical laboratory abnormalities, such as electrolyte abnormalities. Other side effects or adverse events which can be used as a guideline for establishing dose and/or frequency of administration include hematologic adverse events, which include but are not limited to neutropenia, leukopenia, thrombocytopenia, animal, and/or B-cell aplasia and hypogammaglobinemia.

In some embodiments, treatment according to the provided methods can result in a lower rate and/or lower degree of toxicity, toxic outcome or symptom, toxicity-promoting profile, factor, or property, such as a symptom or outcome associated with or indicative of cytokine release syndrome (CRS) or neurotoxicity, such as severe CRS or severe neurotoxicity, for example, compared to administration of other therapies.

In certain embodiments, in the context of genetically engineered cells containing the binding molecules or recombinant receptors, a subject is administered the range of about one million to about 100 billion cells and/or that amount of cells per kilogram of body weight, such as, e.g., about 1 million to about 50 billion cells (e.g., about 5 million cells, about 10 million, about 12.5 million, about 15 million, about 20 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 10 million cells, about 12.5 million cells, about 15 million cells, 20 million cells, about 25 million cells, about 30 million cells, about 40 million cells, about 50 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 150 million cells, about 250 million cells, about 300 million cells, about 350 million cells, about 450 million cells, about 500 million cells, about 600 million cells, about 650 million cells, about 800 million cells, about 900 million cells, about 1 billion cells, about 1.2 billion cells, about 3 billion cells, about 30 billion cells, about 45 billion cells, or about 50 billion cells) or any value in between these ranges and/or per kilogram of body weight. Again, dosages may vary depending on attributes particular to the disease or disorder and/or patient and/or other treatments.

In some embodiments, the methods comprise administering a dose of the engineered cells or a composition comprising a dose of the engineered cells. In some embodiments, the engineered cells or compositions containing engineered cells can be used in a treatment regimen, wherein the treatment regimen comprises administering a dose of the engineered cells or a composition comprising a dose of the engineered cells. In some embodiments, the dose can contain, for example, a particular number or range of recombinant receptor-expressing T cells, total T cells, or total peripheral blood mononuclear cells (PBMCs), such as any number of such cells described herein. In some embodiments, a composition containing a dose of the cells can be administered. In some aspects, the number, amount or proportion of CAR-expressing cells in a cell population or a cell composition can be assessed by detection of a surrogate marker, e.g., by flow cytometry or other means, or by detecting binding of a labelled molecule, such as a labelled antigen, that can specifically bind to the binding molecules or receptors provided herein.

In some embodiments, for example, where the subject is a human, the dose includes more than about 1×10⁶ total recombinant receptor (e.g., CAR)-expressing cells, T cells, or peripheral blood mononuclear cells (PBMCs) and fewer than about 2×10⁹ total recombinant receptor (e.g., CAR)-expressing cells, T cells, or peripheral blood mononuclear cells (PBMCs), e.g., in the range of about 2.5×10⁷ to about 1.2×10⁹ such cells, such as 2.5×10⁷, 5×10⁷, 1.5×10⁸, 3×10⁸, 4.5×10⁸, 8×10⁸, or 1.2×10⁹ total such cells, or the range between any two of the foregoing values. In some embodiments, for example, where the subject is a human, the dose includes more than about 1×10⁶ total recombinant receptor (e.g., CAR)-expressing cells, T cells, or peripheral blood mononuclear cells (PBMCs) and fewer than about 2×10⁹ total recombinant receptor (e.g., CAR)-expressing cells, T cells, or peripheral blood mononuclear cells (PBMCs), e.g., in the range of about 1.0×10⁷ to about 1.2×10⁹ such cells, such as 1.0×10⁷, 1.25×10⁷, 1.5×10⁷, 2.0×10⁷, 2.5×10⁷, 5×10⁷, 7.5×10⁷, 1.5×10⁸, 2.25×10⁸, 3×10⁸, 4.5×10⁸, 6.0×10⁸, 8×10⁸, or 1.2×10⁹ total such cells, or the range between any two of the foregoing values. In some embodiments, for example, where the subject is a human, the dose includes more than about 1×10⁶ total recombinant receptor (e.g., CAR)-expressing cells, T cells, or peripheral blood mononuclear cells (PBMCs) and fewer than about 2×10⁹ total recombinant receptor (e.g., CAR)-expressing cells, T cells, or peripheral blood mononuclear cells (PBMCs), e.g., in the range of about 1.0×10⁷ to about 6.5×10⁸ such cells, about 1.5×10⁷ to about 6.0×10⁸ such cells, about 1.5×10⁷ to about 6.5×10⁸ such cells, about 2.5×10⁷ to about 6.0×10⁸ such cells, or about 5.0×10⁷ to about 6.0×10⁸ such cells.

In some embodiments, the dose of genetically engineered cells comprises between at or about 2.5×10⁷ CAR-expressing T cells, total T cells, or total peripheral blood mononuclear cells (PBMCs), and at or about 1.2×10⁹ CAR-expressing T cells, total T cells, or total PBMCs, between at or about 5.0×10⁷ CAR-expressing T cells and at or about 4.5×10⁸ CAR-expressing T cells, total T cells, or total peripheral blood mononuclear cells (PBMCs), between at or about 1.5×10⁸ CAR-expressing T cells and at or about 3.0×10⁸ CAR-expressing T cells, total T cells, or total PBMCs, each inclusive. In some embodiments, the number is with reference to the total number of CD3+ or CD8+, in some cases also CAR-expressing (e.g. CAR+) cells. In some embodiments, the dose comprises a number of cell from or from about 2.5×10⁷ to or to about 1.2×10⁹ CD3+ or CD8+ total T cells or CD3+ or CD8+ CAR-expressing cells, from or from about 5.0×10⁷ to or to about 4.5×10⁸ CD3+ or CD8+ total T cells or CD3+ or CD8+ CAR-expressing cells, or from or from about 1.5×10⁸ to or to about 3.0×10⁸ CD3+ or CD8+ total T cells or CD3+ or CD8+ CAR-expressing cells, each inclusive.

In some embodiments, the dose of genetically engineered cells comprises between at or about 1.0×10⁷ CAR-expressing T cells, total T cells, or total peripheral blood mononuclear cells (PBMCs), and at or about 1.2×10⁹ CAR-expressing T cells, total T cells, or total PBMCs, between at or about 2.0×10⁷ CAR-expressing T cells and at or about 4.5×10⁸ CAR-expressing T cells, total T cells, or total peripheral blood mononuclear cells (PBMCs), between at or about 1.5×10⁸ CAR-expressing T cells and at or about 3.0×10⁸ CAR-expressing T cells, total T cells, or total PBMCs, each inclusive. In some embodiments, the number is with reference to the total number of CD3+ or CD8+, in some cases also CAR-expressing (e.g. CAR+) cells. In some embodiments, the dose comprises a number of cell from or from about 1.0×10⁷ to or to about 1.2×10⁹ CD3+ or CD8+ total T cells or CD3+ or CD8+ CAR-expressing cells, from or from about 1.5×10⁷ to or to about 1.2×10⁹ CD3+ or CD8+ total T cells or CD3+ or CD8+ CAR-expressing cells, from or from about 2.5×10⁷ to or to about 1.2×10⁹ CD3+ or CD8+ total T cells or CD3+ or CD8+ CAR-expressing cells, from or from about 1.5×10⁷ to or to about 8.0×10⁸ CD3+ or CD8+ total T cells or CD3+ or CD8+ CAR-expressing cells, from or from about 2.5×10⁷ to or to about 8.0×10⁸ CD3+ or CD8+ total T cells or CD3+ or CD8+ CAR-expressing cells, from or from about 1.5×10⁷ to or to about 6.0×10⁸ CD3+ or CD8+ total T cells or CD3+ or CD8+ CAR-expressing cells, from or from about 2.5×10⁷ to or to about 6.0×10⁸ CD3+ or CD8+ total T cells or CD3+ or CD8+ CAR-expressing cells, from or from about 5.0×10⁷ to or to about 6.0×10⁸ CD3+ or CD8+ total T cells or CD3+ or CD8+ CAR-expressing cells, from or from about 5.0×10⁷ to or to about 4.5×10⁸ CD3+ or CD8+ total T cells or CD3+ or CD8+ CAR-expressing cells, or from or from about 1.5×10⁸ to or to about 3.0×10⁸ CD3+ or CD8+ total T cells or CD3+ or CD8+ CAR-expressing cells, each inclusive.

In some embodiments, the T cells of the dose include CD4+ T cells, CD8+ T cells or CD4+ and CD8+ T cells.

In some embodiments, for example, where the subject is human, the CD8+ T cells of the dose, including in a dose including CD4+ and CD8+ T cells, includes between at or about 1×10⁶ and at or about 2×10⁹ total recombinant receptor (e.g., CAR)-expressing CD8+ cells, e.g., in the range of at or about 5×10⁷ to at or about 4.5×10⁸ such cells, such as at or about 2.5×10⁷, at or about 5×10⁷, at or about 1.5×10⁸, at or about 3×10⁸, at or about 4.5×10⁸, at or about 8×10⁸, or at or about 1.2×10⁹ total such cells, or the range between any two of the foregoing values.

In some embodiments, for example, where the subject is human, the CD8+ T cells of the dose, including in a dose including CD4+ and CD8+ T cells, includes between at or about 1×10⁶ and at or about 2×10⁹ total recombinant receptor (e.g., CAR)-expressing CD8+ cells, e.g., in the range of at or about 1×10⁷ to at or about 4.5×10⁸ such cells, such as at or about 1.0×10⁷, at or about 1.25×10⁷, at or about 1.5×10⁷, at or about 2.0×10⁷, at or about 2.5×10⁷, at or about 5×10⁷, at or about 7.5×10⁷, at or about 1.5×10⁸, at or about 3×10⁸, at or about 4.5×10⁸, at or about 6.0×10⁸, at or about 8×10⁸, or at or about 1.2×10⁹ total such cells, or the range between any two of the foregoing values.

In some embodiments, the dose of cells, e.g., recombinant receptor-expressing T cells, is administered to the subject as a single dose or is administered only one time within a period of two weeks, one month, three months, six months, 1 year or more. In some embodiments, the patient is administered multiple doses, and each of the doses or the total dose can be within any of the foregoing values.

In some embodiments, the engineered cells for administration or composition of engineered cells for administration, exhibits properties indicative of or consistent with cell health. In some embodiments, at or about or at least at or about 70, 75, 80, 85, or 90% CAR+ cells of such dose exhibit one or more properties or phenotypes indicative of cell health or biologically active CAR cell, such as absence expression of an apoptotic marker.

In particular embodiments, the phenotype is or includes an absence of apoptosis and/or an indication the cell is undergoing the apoptotic process. Apoptosis is a process of programmed cell death that includes a series of stereotyped morphological and biochemical events that lead to characteristic cell changes and death, including blebbing, cell shrinkage, nuclear fragmentation, chromatin condensation, chromosomal DNA fragmentation, and global mRNA decay. In some aspects, early stages of apoptosis can be indicated by activation of certain caspases, e.g., 2, 8, 9, and 10. In some aspects, middle to late stages of apoptosis are characterized by further loss of membrane integrity, chromatin condensation and DNA fragmentation, include biochemical events such as activation of caspases 3, 6, and 7.

In particular embodiments, the phenotype is negative expression of one or more factors associated with programmed cell death, for example pro-apoptotic factors known to initiate apoptosis, e.g., members of the death receptor pathway, activated members of the mitochondrial (intrinsic) pathway, such as Bcl-2 family members, e.g., Bax, Bad, and Bid, and caspases. In certain embodiments, the phenotype is the absence of an indicator, e.g., an Annexin V molecule or by TUNEL staining, that will preferentially bind to cells undergoing apoptosis when incubated with or contacted to a cell composition. In some embodiments, the phenotype is or includes the expression of one or more markers that are indicative of an apoptotic state in the cell. In some embodiments, the phenotype is lack of expression and/or activation of a caspase, such as caspase 3. In some aspects, activation of caspase-3 is indicative of an increase or revival of apoptosis. In certain embodiments, caspase activation can be detected by known methods. In some embodiments, an antibody that binds specifically to an activated caspase (i.e., binds specifically to the cleaved polypeptide) can be used to detect caspase activation. In particular embodiments, the phenotype is or includes active caspase 3—. In some embodiments, the marker of apoptosis is a reagent that detects a feature in a cell that is associated with apoptosis. In certain embodiments, the reagent is an annexin V molecule.

In some embodiments, the compositions containing the engineered cells for administration contain a certain number or amount of cells that exhibit phenotypes indicative of or consistent with cell health. In some of any embodiments, less than about 25%, 20%, 15%, 10%, 9%, 8%, 7%, 6%, 5%, 4%, 3%, 2% or 1% of the CAR-expressing T cells in the dose of engineered T cells express a marker of apoptosis, optionally Annexin V or active Caspase 3. In some of any embodiments, less than 5%, 4%, 3%, 2% or 1% of the CAR-expressing T cells in the dose of engineered T cells express Annexin V or active Caspase 3.

In some embodiments the cells administered are immune cells engineered to express the BAFF-R-binding recombinant receptor, e.g., CAR. In some embodiments the immune cells are T cells. In some embodiments, the administered cells are CD4+ T cells. In some embodiments the administered cells are CD8+ T cells. In some embodiments, the administered cells are a combination of CD4+ and CD8+ T cells, such as CAR T cells. In some examples the ratio of CD4+ cells to CD8+ cells (CD4: CD8) is 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1.

In some embodiments, the cells, binding molecules, or recombinant receptors are administered as part of a combination treatment, such as simultaneously with or sequentially with, in any order, another therapeutic intervention, such as another antibody or engineered cell or receptor or agent, such as a cytotoxic or therapeutic agent.

The cells, binding molecules and/or recombinant receptors in some embodiments are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cells, binding molecules and/or recombinant receptors are administered prior to the one or more additional therapeutic agents. In some embodiments, the cells, binding molecules and/or recombinant receptors are administered after to the one or more additional therapeutic agents.

In some embodiments, the subject may receive a bridging therapy after leukapheresis and before lymphodepleting chemotherapy. A treating physician can determine if bridging therapy is necessary, for example for disease control, during manufacturing of the provided composition or cells. In some embodiments, bridging therapies do not include biological agents, such as antibodies (e.g., Daratumumab). In some embodiments, bridging therapies are discontinued prior to initiation of lymphodepletion. In some embodiments, bridging therapies are discontinued 1 day, 2 days 3 days, 4 days, 5 days, 7 days, 10 days, 14 days, 21 days, 28 days, 45 days, or 60 days before lymphodepletion.

Once the cells are administered to a mammal (e.g., a human), the biological activity of the engineered cell populations is measured by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable known methods, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells also can be measured by assaying expression and/or secretion of certain cytokines, such as CD 107a, IFNγ, IL-2, and TNF. In some aspects the biological activity is measured by assessing clinical outcome, such as reduction in tumor burden or load.

In certain embodiments, engineered cells are modified in any number of ways, such that their therapeutic or prophylactic efficacy is increased. For example, the engineered CAR expressed by the population in some embodiments are conjugated either directly or indirectly through a linker to a targeting moiety. The practice of conjugating compounds, e.g., the CAR to targeting moieties is known. See, for instance, Wadwa et al., J. Drug Targeting 3: 111 (1995), and U.S. Pat. No. 5,087,616.

### B. Combination Therapy

Also provided are methods of combination therapy that include administration and uses, such as therapeutic and prophylactic uses, of the BAFF-R- and CD19-binding recombinant receptors (e.g., bispecific CARs), engineered cells expressing the recombinant receptors (e.g., CARs), plurality of engineered cells expressing the receptors, and/or compositions comprising the same.

In some embodiments, the BAFF-R- and CD19-binding recombinant receptor (e.g., bispecific CAR), and/or engineered cells expressing said molecules (e.g., recombinant receptor) described herein are administered as part of a combination treatment or combination therapy, such as simultaneously with, sequentially with, or intermittently with, in any order, one or more additional therapeutic intervention. In some embodiments, the one or more additional therapeutic intervention includes, for example, an antibody, an engineered cell, a receptor and/or an agent, such as a cell expressing a recombinant receptor, and/or cytotoxic or therapeutic agent, e.g., a chemotherapeutic agent. In some embodiments, the combination therapy includes administration of one or more additional agents, therapies and/or treatments, e.g., any of the additional agents, therapy and/or treatments described herein. In some embodiments, the combination therapy includes administration of one or more additional agents for treatment or therapy, such as an immunomodulatory agent, immune checkpoint inhibitor, adenosine pathway or adenosine receptor antagonist or agonist and kinase inhibitors. In some embodiments, the combination treatment or combination therapy includes an additional treatment, such as a surgical treatment, transplant, and/or radiation therapy. Also provided are methods of combination treatment or combination therapy that includes BAFF-R- and CD19-binding recombinant receptors (e.g., bispecific CARs), cells and/or compositions described herein and one or more additional therapeutic interventions.

In some embodiments, the additional agent for combination treatment or combination therapy enhances, boosts and/or promotes the efficacy and/or safety of the therapeutic effect of binding molecules, recombinant receptors, cells and/or compositions. In some embodiments, the additional agent enhances or improves the efficacy, survival or persistence of the administered cells, e.g., cells expressing the binding molecule or a recombinant receptor. In some embodiments, the additional agent is selected from among a protein phosphatase inhibitor, a kinase inhibitor, a cytokine, an immunomodulator, or an agent that decreases the level or activity of a regulatory T (Treg) cell. In some embodiments, the additional agent enhances safety, by virtue of reducing or ameliorating adverse effects of the administered binding molecules, recombinant receptors, cells and/or compositions. In some embodiments, the additional agent can treat the same disease, condition or a comorbidity. In some embodiments, the additional agent can ameliorate, reduce or eliminate one or more toxicities, adverse effects or side effects that are associated with administration of the recombinant receptors, cells and/or compositions, e.g., CAR-expressing cells.

In some embodiments, pain management medication such as acetaminophen, or antihistamine, such as diphenhydramine can be administered prior to, during or after administration of the recombinant receptor, cell or composition provided herein, to ameliorate or reduce or eliminate minor side effects associated with treatment. In some examples, red blood cell and platelet transfusions, and/or colony-stimulating factors can be administered reduce or eliminate one or more toxicities, adverse effects or side effects that are associated with administration of the recombinant receptors, cells and/or compositions, e.g., CAR-expressing cells. In some embodiments, prophylactic or empiric anti-infective agents (e.g., trimethoprim/sulfamethoxazole for pneumocystis pneumonia [PCP] prophylaxis, broad spectrum antibiotics, antifungals, or antiviral agents for febrile neutropenia) can be administered to treat side-effects resulting from treatment. In some examples, when necessary, prophylaxis may be provided to treat lymphopenia and/or neutropenia occurring as a result of treatment.

In some embodiments, the additional therapy, treatment or agent includes chemotherapy, radiation therapy, surgery, transplantation, adoptive cell therapy, antibodies, cytotoxic agents, chemotherapeutic agents, cytokines, growth inhibitory agents, anti-hormonal agents, kinase inhibitors, anti-angiogenic agents, cardioprotectants, immunostimulatory agents, immunosuppressive agents, immune checkpoint inhibitors, antibiotics, angiogenesis inhibitors, metabolic modulators or other therapeutic agents or any combination thereof. In some embodiments, the additional agent is a protein, a peptide, a nucleic acid, a small molecule agent, a cell, a toxin, a lipid, a carbohydrate or combinations thereof, or any other type of therapeutic agent, e.g., radiation. In some embodiments, the additional therapy, agent or treatment includes surgery, chemotherapy, radiation therapy, transplantation, administration of cells expressing a recombinant receptor, e.g., CAR, kinase inhibitor, immune checkpoint inhibitor, mTOR pathway inhibitor, immunosuppressive agents, immunomodulators, antibodies, immunoablative agents, antibodies and/or antigen binding fragments thereof, antibody conjugates, other antibody therapies, cytotoxins, steroids, cytokines, peptide vaccines, hormone therapy, antimetabolites, metabolic modulators, drugs that inhibit either the calcium dependent phosphatase calcineurin or the p70S6 kinase FK506) or inhibit the p70S6 kinase, alkylating agents, anthracyclines, vinca alkaloids, proteasome inhibitors, GITR agonists, protein tyrosine phosphatase inhibitors, protein kinase inhibitors, an oncolytic virus, and/or other types of immunotherapy. In some embodiments, the additional agent or treatment is bone marrow transplantation, T cell ablative therapy using chemotherapy agents such as, fludarabine, external-beam radiation therapy (XRT), cyclophosphamide, and/or antibody therapy.

In some embodiments, the cells, BAFF-R- and CD19-binding recombinant receptors and/or compositions, e.g., CAR-expressing cells, are administered in combination with other engineered cells, e.g., other CAR-expressing cells. In some embodiments, the cells, BAFF-R- and CD19-binding recombinant receptors and/or compositions, e.g., CAR-expressing cells, are administered in combination with an additional agent. In some embodiments, the cells, BAFF-R- and CD19-binding recombinant receptors and/or compositions, e.g., CAR-expressing cells, are administered in combination with other engineered cells, e.g., other CAR-expressing cells, as well as in combination with an additional agent. In some embodiments, the additional agent is a kinase inhibitor, e.g., an inhibitor of Bruton's tyrosine kinase (Btk), e.g., ibrutinib. In some embodiments, the additional agent is an adenosine pathway or adenosine receptor antagonist or agonist. In some embodiments, the additional agent is an immunomodulator such as thalidomide or a thalidomide derivative (e.g., lenalidomide). In some embodiments, the additional agent is a gamma secretase inhibitor, such as a gamma secretase inhibitor that inhibits or reduces intramembrane cleavage of a target of a gamma secretase, e.g., CD19, on a cell (such as a tumor/cancer cell). In some embodiments, the additional therapy, agent or treatment is a cytotoxic or chemotherapy agent, a biologic therapy (e.g., antibody, e.g., monoclonal antibody, or cellular therapy), or an inhibitor (e.g., kinase inhibitor).

In some embodiments, the additional agent is a chemotherapeutic agent. Exemplary chemotherapeutic agents include an anthracycline (e.g., doxorubicin, such as liposomal doxorubicin); a vinca alkaloid (e.g., vinblastine, vincristine, vindesine, vinorelbine); an alkylating agent (e.g., cyclophosphamide, decarbazine, melphalan, ifosfamide, temozolomide); an immune cell antibody (e.g., alemtuzumab, gemtuzumab, rituximab, tositumomab); an antimetabolite (including, e.g., folic acid antagonists, pyrimidine analogs, purine analogs and adenosine deaminase inhibitors such as fludarabine); a TNFR glucocorticoid induced TNFR related protein (GITR) agonist; a proteasome inhibitor (e.g., aclacinomycin A, gliotoxin or bortezomib); an immunomodulatory such as thalidomide or a thalidomide derivative (e.g., lenalidomide).

In some embodiments, the additional therapy or treatment is cell therapy, e.g., adoptive cell therapy. In some embodiments, the additional therapy includes administration of engineered cells, e.g., additional CAR-expressing cell. In some embodiments, the additional engineered cell is a CAR-expressing cell that expresses the same or different recombinant receptor as the engineered cells provided herein, cells. In some embodiments, the recombinant receptor, e.g., CAR, expressed on the additional engineered cell, recognizes a different antigen and/or epitope. In some embodiments, the recombinant receptor, e.g., CAR, expressed on the additional engineered cell, recognizes a different epitope of the same antigen as the recombinant receptors described herein, e.g., BAFF-R or CD19. In some embodiments, the recombinant receptor, e.g., CAR, expressed on the additional engineered cell, recognizes a different antigen, e.g., a different tumor antigen or combination of antigens. For example, in some embodiments, the recombinant receptor, e.g., CAR, expressed on the additional engineered cell, targets cancer cells that express early lineage markers, e.g., cancer stem cells, while other CAR-expressing cells target cancer cells that express later lineage markers. In such embodiments, the additional engineered cell is administered prior to, concurrently with, or after administration (e.g., infusion) of the CAR-expressing cells described herein. In some embodiments, the additional engineered cell expresses an allogeneic CAR.

In some embodiments, the configurations of one or more of the CAR molecules comprise a primary intracellular signaling domain and two or more, e.g., 2, 3, 4, or 5 or more, costimulatory signaling domains. In some embodiments, the one or more of the CAR molecules may have the same or a different primary intracellular signaling domain, the same or different costimulatory signaling domains, or the same number or a different number of costimulatory signaling domains. In some embodiments, the one or more of the CAR molecules can be configured as a split CAR, in which one of the CAR molecules comprises an antigen binding domain and a costimulatory domain (e.g., 4-1BB), while the other CAR molecule comprises an antigen binding domain and a primary intracellular signaling domain (e.g., CD3 zeta).

In some embodiments, the additional agent is any of the cells engineered to express one or more of the BAFF-R- and CD19-binding molecules and/or cells engineered to express additional binding molecules, e.g., recombinant receptors, e.g., CAR, that target a different antigen. In some embodiments, the additional agent includes any of the cells or plurality of cells described herein, e.g., in Section III. In some embodiments, the additional agent is a cell engineered to express a recombinant receptor, e.g., CAR, targeting a different epitope and/or antigen, e.g., a different antigen associated with a disease or condition. In some embodiments, the additional agent is a cell engineered to express a recombinant receptor, e.g., CAR, targeting a second or additional antigen expressed in cancer.

In some embodiments, the additional agent is an immunomodulatory agent. In some embodiments, the combination therapy includes an immunomodulatory agent that can stimulate, amplify and/or otherwise enhance an anti-tumor immune response, e.g., anti-tumor immune response from the administered engineered cells, such as by inhibiting immunosuppressive signaling or enhancing immunostimulant signaling. In some embodiments, the immunomodulatory agent is a peptide, protein or is a small molecule. In some embodiments, the protein can be a fusion protein or a recombinant protein. In some embodiments, the immunomodulatory agent binds to an immunologic target, such as a cell surface receptor expressed on immune cells, such a T cells, B cells or antigen-presenting cells. For example, in some embodiments, the immunomodulatory agent is an antibody or antigen-binding antibody fragment, a fusion protein, a small molecule or a polypeptide. In some embodiments, the recombinant receptors, cells and/or compositions are administered in combination with an additional agent that is an antibody or an antigen-binding fragment thereof, such as a monoclonal antibody.

In some embodiments, the immunomodulatory agent blocks, inhibits or counteracts a component of the immune checkpoint pathway. The immune system has multiple inhibitory pathways that are involved in maintaining self-tolerance and for modulating immune responses. Tumors can use certain immune-checkpoint pathways as a major mechanism of immune resistance, particularly against T cells that are specific for tumor antigens (Pardoll (2012) Nature Reviews Cancer 12:252-264), e.g., engineered cells such as CAR-expressing cells. Because many such immune checkpoints are initiated by ligand-receptor interactions, they can be readily blocked by antibodies against the ligands and/or their receptors.

Therefore, therapy with antagonistic molecules blocking an immune checkpoint pathway, such as small molecules, nucleic acid inhibitors (e.g., RNAi) or antibody molecules, are becoming promising avenues of immunotherapy for cancer and other diseases. In contrast to the majority of anti-cancer agents, checkpoint inhibitors do not necessarily target tumor cells directly, but rather target lymphocyte receptors or their ligands in order to enhance the endogenous antitumor activity of the immune system.

As used herein, the term “immune checkpoint inhibitor” refers to molecules that totally or partially reduce, inhibit, interfere with or modulate one or more checkpoint proteins. Checkpoint proteins regulate T-cell activation or function. These proteins are responsible for co-stimulatory or inhibitory interactions of T-cell responses. Immune checkpoint proteins regulate and maintain self-tolerance and the duration and amplitude of physiological immune responses. In some embodiments, the subject can be administered an additional agent that can enhance or boost the immune response, e.g., immune response effected by the BAFF-R- and CD19-binding recombinant receptors, cells and/or compositions provided herein, against a disease or condition, e.g., a cancer, such as any described herein.

Immune checkpoint inhibitors include any agent that blocks or inhibits in a statistically significant manner, the inhibitory pathways of the immune system. Such inhibitors may include small molecule inhibitors or may include antibodies, or antigen binding fragments thereof, that bind to and block or inhibit immune checkpoint receptors, ligands and/or receptor-ligand interaction. In some embodiments, modulation, enhancement and/or stimulation of particular receptors can overcome immune checkpoint pathway components. Illustrative immune checkpoint molecules that may be targeted for blocking, inhibition, modulation, enhancement and/or stimulation include, but are not limited to, PD-1 (CD279), PD-L1 (CD274, B7-H1), PDL2 (CD273, B7-DC), CTLA-4, LAG-3 (CD223), TIM-3, 4-1BB (CD137), 4-1BBL (CD137L), GITR (TNFRSF18, AITR), CD40, OX40 (CD134, TNFRSF4), CXCR2, tumor associated antigens (TAA), B7-H3, B7-H4, BTLA, HVEM, GAL9, B7H3, B7H4, VISTA, KIR, 2B4 (belongs to the CD2 family of molecules and is expressed on all NK, γδ, and memory CD8+ (αβ) T cells), CD160 (also referred to as BY55), CGEN-15049, CEACAM (e.g., CEACAM-1, CEACAM-3 and/or CEACAM-5), TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and a transforming growth factor receptor (TGFR; e.g., TGFR beta). Immune checkpoint inhibitors include antibodies, or antigen binding fragments thereof, or other binding proteins, that bind to and block or inhibit and/or enhance or stimulate the activity of one or more of any of the said molecules.

Exemplary immune checkpoint inhibitors include Tremelimumab (CTLA-4 blocking antibody, also known as ticilimumab, CP-675,206), anti-OX40, PD-L1 monoclonal antibody (Anti-B7-H1; MEDI4736), MK-3475 (PD-1 blocker), nivolumab (anti-PD-1 antibody), CT-011 (anti-PD-1 antibody), BY55 monoclonal antibody, AMP224 (anti-PD-L1 antibody), BMS-936559 (anti-PD-L1 antibody), MPLDL3280A (anti-PD-L1 antibody), MSB0010718C (anti-PD-L1 antibody) and ipilimumab (anti-CTLA-4 antibody, also known as Yervoy®, MDX-010 and MDX-101). Exemplary immunomodulatory antibodies include, but are not limited to, Daclizumab (Zenapax), Bevacizumab (Avastin®), Basiliximab, Ipilimumab, Nivolumab, pembrolizumab, MPDL3280A, Pidilizumab (CT-011), MK-3475, BMS-936559, MPDL3280A (Atezolizumab), tremelimumab, IMP321, BMS-986016, LAG525, urelumab, PF-05082566, TRX518, MK-4166, dacetuzumab (SGN-40), lucatumumab (HCD122), SEA-CD40, CP-870, CP-893, MEDI6469, MEDI6383, MOXR0916, AMP-224, MSB0010718C (Avelumab), MEDI4736, PDR001, rHIgM12B7, Ulocuplumab, BKT140, Varlilumab (CDX-1127), ARGX-110, MGA271, lirilumab (BMS-986015, IPH2101), IPH2201, ARGX-115, Emactuzumab, CC-90002 and MNRP1685A or an antibody-binding fragment thereof. Other exemplary immunomodulators include, e.g., afutuzumab (available from Roche®); pegfilgrastim (Neulasta®); lenalidomide (CC-5013, Revlimid®); thalidomide (Thalomid®), actimid (CC4047); and IRX-2 (mixture of human cytokines including interleukin 1, interleukin 2, and interferon gamma, CAS 951209-71-5, available from IRX Therapeutics).

Programmed cell death 1 (PD-1) is an immune checkpoint protein that is expressed in B cells, NK cells, and T cells (Shinohara et al., 1995, Genomics 23:704-6; Blank et al., 2007, Cancer Immunol Immunother 56:739-45; Finger et al., 1997, Gene 197:177-87; Pardoll (2012) Nature Reviews Cancer 12:252-264). The major role of PD-1 is to limit the activity of T cells in peripheral tissues during inflammation in response to infection, as well as to limit autoimmunity. PD-1 expression is induced in activated T cells and binding of PD-1 to one of its endogenous ligands acts to inhibit T-cell activation by inhibiting stimulatory kinases. PD-1 also acts to inhibit the TCR “stop signal”. PD-1 is highly expressed on Treg cells and may increase their proliferation in the presence of ligand (Pardoll (2012) Nature Reviews Cancer 12:252-264). Anti-PD 1 antibodies have been used for treatment of melanoma, non-small-cell lung cancer, bladder cancer, prostate cancer, colorectal cancer, head and neck cancer, triple-negative breast cancer, leukemia, lymphoma and renal cell cancer (Topalian et al., 2012, N Engl J Med 366:2443-54; Lipson et al., 2013, Clin Cancer Res 19:462-8; Berger et al., 2008, Clin Cancer Res 14:3044-51; Gildener-Leapman et al., 2013, Oral Oncol 49:1089-96; Menzies & Long, 2013, Ther Adv Med Oncol 5:278-85). Exemplary anti-PD-1 antibodies include nivolumab (Opdivo by BMS), pembrolizumab (Keytruda by Merck), pidilizumab (CT-011 by Cure Tech), lambrolizumab (MK-3475 by Merck), and AMP-224 (Merck), nivolumab (also referred to as Opdivo, BMS-936558 or MDX1106; Bristol-Myers Squibb) is a fully human IgG4 monoclonal antibody which specifically blocks PD-1. Nivolumab (clone 5C4) and other human monoclonal antibodies that specifically bind to PD-1 are described in U.S. Pat. No. 8,008,449 and WO2006/121168. Pidilizumab (CT-011; Cure Tech) is a humanized IgG1k monoclonal antibody that binds to PD-1. Pidilizumab and other humanized anti-PD-1 monoclonal antibodies are described in WO2009/101611. Pembrolizumab (formerly known as lambrolizumab, and also referred to as Keytruda, MK03475; Merck) is a humanized IgG4 monoclonal antibody that binds to PD-1. Pembrolizumab and other humanized anti-PD-1 antibodies are described in U.S. Pat. No. 8,354,509 and WO2009/114335. Other anti-PD-1 antibodies include AMP 514 (Amplimmune), among others, e.g., anti-PD-1 antibodies described in U.S. Pat. No. 8,609,089, US2010028330, US 20120114649 and/or US20150210769. AMP-224 (B7-DCIg; Amplimmune; e.g., described in WO2010/027827 and WO2011/066342), is a PD-L2 Fc fusion soluble receptor that blocks the interaction between PD-1 and B7-H1.

PD-L1 (also known as CD274 and B7-H1) and PD-L2 (also known as CD273 and B7-DC) are ligands for PD-1, found on activated T cells, B cells, myeloid cells, macrophages, and some types of tumor cells. Anti-tumor therapies have focused on anti-PD-L1 antibodies. The complex of PD-1 and PD-L1 inhibits proliferation of CD8+ T cells and reduces the immune response (Topalian et al., 2012, N Engl J Med 366:2443-54; Brahmer et al., 2012, N Eng J Med 366:2455-65). Anti-PD-L1 antibodies have been used for treatment of non-small cell lung cancer, melanoma, colorectal cancer, renal-cell cancer, pancreatic cancer, gastric cancer, ovarian cancer, breast cancer, and hematologic malignancies (Brahmer et al., 2012, N Eng J Med 366:2455-65; Ott et al., 2013, Clin Cancer Res 19:5300-9; Radvanyi et al., 2013, Clin Cancer Res 19:5541; Menzies & Long, 2013, Ther Adv Med Oncol 5:278-85; Berger et al., 2008, Clin Cancer Res 14:13044-51). Exemplary anti-PD-L1 antibodies include MDX-1105 (Medarex), MEDI4736 (Medimmune) MPDL3280A (Genentech), BMS-935559 (Bristol-Myers Squibb) and MSB0010718C. MEDI4736 (Medimmune) is a human monoclonal antibody that binds to PD-L1, and inhibits interaction of the ligand with PD-1. MDPL3280A (Genentech/Roche) is a human Fc optimized IgG1 monoclonal antibody that binds to PD-L1. MDPL3280A and other human monoclonal antibodies to PD-L1 are described in U.S. Pat. No. 7,943,743 and U.S. Publication No. 20120039906. Other anti-PD-L1 binding agents include YW243.55.S70 (see WO2010/077634) and MDX-1105 (also referred to as BMS-936559, and, e.g., anti-PD-L1 binding agents described in WO2007/005874).

Cytotoxic T-lymphocyte-associated antigen (CTLA-4), also known as CD152, is a co-inhibitory molecule that functions to regulate T-cell activation. CTLA-4 is a member of the immunoglobulin superfamily that is expressed exclusively on T-cells. CTLA-4 acts to inhibit T-cell activation and is reported to inhibit helper T-cell activity and enhance regulatory T-cell immunosuppressive activity. Although the precise mechanism of action of CTLA-4 remains under investigation, it has been suggested that it inhibits T cell activation by outcompeting CD28 in binding to CD80 and CD86, as well as actively delivering inhibitor signals to the T cell (Pardoll (2012) Nature Reviews Cancer 12:252-264). Anti-CTLA-4 antibodies have been used in clinical trials for the treatment of melanoma, prostate cancer, small cell lung cancer, non-small cell lung cancer (Robert & Ghiringhelli, 2009, Oncologist 14:848-61; Ott et al., 2013, Clin Cancer Res 19:5300; Weber, 2007, Oncologist 12:864-72; Wada et al., 2013, J Transl Med 11:89). A significant feature of anti-CTLA-4 is the kinetics of anti-tumor effect, with a lag period of up to 6 months after initial treatment required for physiologic response. In some cases, tumors may actually increase in size after treatment initiation, before a reduction is seen (Pardoll (2012) Nature Reviews Cancer 12:252-264). Exemplary anti-CTLA-4 antibodies include ipilimumab (Bristol-Myers Squibb) and tremelimumab (Pfizer). Ipilimumab has recently received FDA approval for treatment of metastatic melanoma (Wada et al., 2013, J Transl Med 11:89).

Lymphocyte activation gene-3 (LAG-3), also known as CD223, is another immune checkpoint protein. LAG-3 has been associated with the inhibition of lymphocyte activity and in some cases the induction of lymphocyte anergy. LAG-3 is expressed on various cells in the immune system including B cells, NK cells, and dendritic cells. LAG-3 is a natural ligand for the MHC class II receptor, which is substantially expressed on melanoma-infiltrating T cells including those endowed with potent immune-suppressive activity. Exemplary anti-LAG-3 antibodies include Relatlimab (BMS-986016) (Bristol-Myers Squib), which is a monoclonal antibody that targets LAG-3. IMP701 (Immutep) is an antagonist LAG-3 antibody and IMP731 (Immutep and GlaxoSmithKline) is a depleting LAG-3 antibody. Other LAG-3 inhibitors include IMP321 (Immutep), which is a recombinant fusion protein of a soluble portion of LAG-3 and Ig that binds to MHC class II molecules and activates antigen presenting cells (APC). Other antibodies are described, e.g., in WO2010/019570 and US 2015/0259420.

T-cell immunoglobulin domain and mucin domain-3 (TIM-3), initially identified on activated Th1 cells, has been shown to be a negative regulator of the immune response. Blockade of TIM-3 promotes T-cell mediated anti-tumor immunity and has anti-tumor activity in a range of mouse tumor models. Combinations of TIM-3 blockade with other immunotherapeutic agents such as TSR-042, anti-CD137 antibodies and others, can be additive or synergistic in increasing anti-tumor effects. TIM-3 expression has been associated with a number of different tumor types including melanoma, NSCLC and renal cancer, and additionally, expression of intratumoral TIM-3 has been shown to correlate with poor prognosis across a range of tumor types including NSCLC, cervical, and gastric cancers. Blockade of TIM-3 is also of interest in promoting increased immunity to a number of chronic viral diseases. TIM-3 has also been shown to interact with a number of ligands including galectin-9, phosphatidylserine and HMGB1, although which of these, if any, are relevant in regulation of anti-tumor responses is not clear at present. In some embodiments, antibodies, antibody fragments, small molecules, or peptide inhibitors that target TIM-3 can bind to the IgV domain of TIM-3 to inhibit interaction with its ligands. Exemplary antibodies and peptides that inhibit TIM-3 are described in US 2015/0218274, WO2013/006490 and US 2010/0247521. Other anti-TIM-3 antibodies include humanized versions of RMT3-23 (Ngiow et al., 2011, Cancer Res, 71:3540-3551), and clone 8B.2C12 (Monney et al., 2002, Nature, 415:536-541). Bi-specific antibodies that inhibit TIM-3 and PD-1 are described in US 2013/0156774.

In some embodiments, the additional agent is a CEACAM inhibitor (e.g., CEACAM-1, CEACAM-3, and/or CEACAM-5 inhibitor). In some embodiments, the inhibitor of CEACAM is an anti-CEACAM antibody molecule. Exemplary anti-CEACAM-1 antibodies are described in WO 2010/125571, WO 2013/082366 WO 2014/059251 and WO 2014/022332, e.g., a monoclonal antibody 34B1, 26H7, and 5F4; or a recombinant form thereof, as described in, e.g., US 2004/0047858, U.S. Pat. No. 7,132,255 and WO 99/052552. In some embodiments, the anti-CEACAM antibody binds to CEACAM-5 as described in, e.g., Zheng et al. PLOS One. (2011) 6(6): e21146), or cross reacts with CEACAM-1 and CEACAM-5 as described in, e.g., WO 2013/054331 and US 2014/0271618.

4-1BB, also known as CD137, is transmembrane glycoprotein belonging to the TNFR superfamily. 4-1BB receptors are present on activated T cells and B cells and monocytes. An exemplary anti-4-1BB antibody is urelumab (BMS-663513), which has potential immunostimulatory and antineoplastic activities.

Tumor necrosis factor receptor superfamily, member 4 (TNFRSF4), also known as OX40 and CD134, is another member of the TNFR superfamily. OX40 is not constitutively expressed on resting naïve T cells and acts as a secondary co-stimulatory immune checkpoint molecule. Exemplary anti-OX40 antibodies are MEDI6469 and MOXR0916 (RG7888, Genentech).

In some embodiments, the additional agent includes a molecule that decreases the regulatory T cell (Treg) population. Methods that decrease the number of (e.g., deplete) Treg cells are known in the art and include, e.g., CD25 depletion, cyclophosphamide administration, and modulating Glucocorticoid-induced TNFR family related gene (GITR) function. GITR is a member of the TNFR superfamily that is upregulated on activated T cells, which enhances the immune system. Reducing the number of Treg cells in a subject prior to apheresis or prior to administration of engineered cells, e.g., CAR-expressing cells, can reduce the number of unwanted immune cells (e.g., Tregs) in the tumor microenvironment and reduces the subject's risk of relapse. In some embodiments, the additional agent includes a molecule targeting GITR and/or modulating GITR functions, such as a GITR agonist and/or a GITR antibody that depletes regulatory T cells (Tregs). In some embodiments, the additional agent includes cyclophosphamide. In some embodiments, the GITR binding molecule and/or molecule modulating GITR function (e.g., GITR agonist and/or Treg depleting GITR antibodies) is administered prior to the engineered cells, e.g., CAR-expressing cells. For example, in some embodiments, the GITR agonist can be administered prior to apheresis of the cells. In some embodiments, cyclophosphamide is administered to the subject prior to administration (e.g., infusion or re-infusion) of the engineered cells, e.g., CAR-expressing cells or prior to apheresis of the cells. In some embodiments, cyclophosphamide and an anti-GITR antibody are administered to the subject prior to administration (e.g., infusion or re-infusion) of the engineered cells, e.g., CAR-expressing cells or prior to apheresis of the cells.

In some embodiments, the additional agent is a GITR agonist. Exemplary GITR agonists include, e.g., GITR fusion proteins and anti-GITR antibodies (e.g., bivalent anti-GITR antibodies) such as, e.g., a GITR fusion protein described in U.S. Pat. No. 6,111,090, European Patent No. 090505B 1, U.S. Pat. No. 8,586,023, PCT Publication Nos.: WO 2010/003118 and 2011/090754, or an anti-GITR antibody described, e.g., in U.S. Pat. No. 7,025,962, European Patent No. 1947183B 1, U.S. Pat. Nos. 7,812,135, 8,388,967, 8,591,886, European Patent No. EP 1866339, PCT Publication No. WO 2011/028683, PCT Publication No. WO 2013/039954, PCT Publication No.

WO2005/007190, PCT Publication No. WO 2007/133822, PCT Publication No.

WO2005/055808, PCT Publication No. WO 99/40196, PCT Publication No. WO 2001/03720, PCT Publication No. WO99/20758, PCT Publication No. WO2006/083289, PCT Publication No. WO 2005/115451, U.S. Pat. No. 7,618,632, and PCT Publication No. WO 2011/051726. An exemplary anti-GITR antibody is TRX518.

In some embodiments, the additional agent enhances tumor infiltration or transmigration of the administered cells, e.g., CAR-expressing cells. For example, in some embodiments, the additional agent stimulates CD40, such as CD40L, e.g., recombinant human CD40L. Cluster of differentiation 40 (CD40) is also a member of the TNFR superfamily. CD40 is a costimulatory protein found on antigen-presenting cells and mediates a broad variety of immune and inflammatory responses. CD40 is also expressed on some malignancies, where it promotes proliferation. Exemplary anti-CD40 antibodies are dacetuzumab (SGN-40), lucatumumab (Novartis, antagonist), SEA-CD40 (Seattle Genetics), and CP-870,893. In some embodiments, the additional agent that enhances tumor infiltration includes tyrosine kinase inhibitor sunitnib, heparanase, and/or chemokine receptors such as CCR2, CCR4, and CCR7.

In some embodiments, the additional agent includes thalidomide drugs or analogs thereof and/or derivatives thereof, such as lenalidomide, pomalidomide or apremilast. See, e.g., Bertilaccio et al., Blood (2013) 122:4171, Otahal et al., Oncoimmunology (2016) 5(4): e1115940; Fecteau et al., Blood (2014) 124(10): 1637-1644 and Kuramitsu et al., Cancer Gene Therapy (2015) 22:487-495). Lenalidomide ((RS)-3-(4-Amino-1-oxo-1,3-dihydro-2H-isoindol-2-yl) piperidine-2,6-dione; also known as Revlimid) is a synthetic derivative of thalidomide, and has multiple immunomodulatory effects, including enforcement of immune synapse formation between T cell and antigen presenting cells (APCs). For example, in some cases, lenalidomide modulates T cell responses and results in increased interleukin (IL)-2 production in CD4+ and CD8+ T cells, induces the shift of T helper (Th) responses from Th2 to Th1, inhibits expansion of regulatory subset of T cells (Tregs), and improves functioning of immunological synapses in follicular lymphoma and chronic lymphocytic leukemia (CLL) (Otahal et al., Oncoimmunology (2016) 5(4): e1115940). Lenalidomide also can enhance T-cell proliferation and interferon-γ production in response to activation of T cells via CD3 ligation or dendritic cell-mediated activation. Lenalidomide can also induce malignant B cells to express higher levels of immunostimulatory molecules such as CD80, CD86, HLA-DR, CD95, and CD40 (Fecteau et al., Blood (2014) 124(10): 1637-1644). In some embodiments, lenalidomide is administered at a dosage of from about 1 mg to about 20 mg daily, e.g., from about 1 mg to about 10 mg, from about 2.5 mg to about 7.5 mg, from about 5 mg to about 15 mg, such as about 5 mg, 10 mg, 15 mg or 20 mg daily. In some embodiments, lenalidomide is administered at a dose of from about 10 μg/kg to 5 mg/kg, e.g., about 100 μg/kg to about 2 mg/kg, about 200 μg/kg to about 1 mg/kg, about 400 μg/kg to about 600 μg/kg, such as about 500 μg/kg. In some embodiments, rituximab is administered at a dosage of about 350-550 mg/m² (e.g., 350-375, 375-400, 400-425, 425-450, 450-475, or 475-500 mg/m²), e.g., intravenously. In some embodiments, lenalidomide is administered at a low dose.

In some embodiments, the additional agent is a B-cell inhibitor. In some embodiments, the additional agent is one or more B-cell inhibitors selected from among inhibitors of CD10, CD20, CD22, CD34, CD123, CD79a, CD79b, CD179b, FLT-3, or ROR1, or a combination thereof. In some embodiments, the B-cell inhibitor is an antibody (e.g., a mono- or bispecific antibody) or an antigen binding fragment thereof. In some embodiments, the additional agent is an engineered cell expressing recombinant receptors that target B-cell targets, e.g., CD10, CD20, CD22, CD34, CD123, CD79a, CD79b, CD179b, FLT-3, or ROR1.

In some embodiments, the additional agent is a CD20 inhibitor, e.g., an anti-CD20 antibody (e.g., an anti-CD20 mono- or bi-specific antibody) or a fragment thereof. Exemplary anti-CD20 antibodies include but are not limited to rituximab, ofatumumab, ocrelizumab (also known as GA101 or RO5072759), veltuzumab, obinutuzumab, TRU-015 (Trubion Pharmaceuticals), ocaratuzumab (also known as AME-133v or ocaratuzumab), and Pro131921 (Genentech). See, e.g., Lim et al. Haematologica. (2010) 95(1): 135-43. In some embodiments, the anti-CD20 antibody comprises rituximab. Rituximab is a chimeric mouse/human monoclonal antibody IgG1 kappa that binds to CD20 and causes cytolysis of a CD20 expressing cell. In some embodiments, the additional agent includes rituximab. In some embodiments, the CD20 inhibitor is a small molecule.

In some embodiments, the additional agent is a CD22 inhibitor, e.g., an anti-CD22 antibody (e.g., an anti-CD22 mono- or bi-specific antibody) or a fragment thereof. Exemplary anti-CD22 antibodies include epratuzumab and RFB4. In some embodiments, the CD22 inhibitor is a small molecule. In some embodiments, the antibody is a monospecific antibody, optionally conjugated to a second agent such as a chemotherapeutic agent. For instance, in some embodiments, the antibody is an anti-CD22 monoclonal antibody-MMAE conjugate (e.g., DCDT2980S). In some embodiments, the antibody is an scFv of an anti-CD22 antibody, e.g., an scFv of antibody RFB4. In some embodiments, the scFv is fused to all of or a fragment of Pseudomonas exotoxin-A (e.g., BL22). In some embodiments, the scFv is fused to all of or a fragment of (e.g., a 38 kDa fragment of) Pseudomonas exotoxin-A (e.g., moxetumomab pasudotox). In some embodiments, the anti-CD22 antibody is an anti-CD19/CD22 bispecific antibody, optionally conjugated to a toxin. For instance, in some embodiments, the anti-CD22 antibody comprises an anti-CD19/CD22 bispecific portion, (e.g., two scFv ligands, recognizing human CD19 and CD22) optionally linked to all of or a portion of diphtheria toxin (DT), e.g., first 389 amino acids of diphtheria toxin (DT), DT 390, e.g., a ligand-directed toxin such as DT2219ARL). In some embodiments, the bispecific portion (e.g., anti-CD 19/anti-CD22) is linked to a toxin such as deglycosylated ricin A chain (e.g., Combotox).

In some embodiments, the immunomodulatory agent is a cytokine. In some embodiments, the immunomodulatory agent is a cytokine or is an agent that induces increased expression of a cytokine in the tumor microenvironment. Cytokines have important functions related to T cell expansion, differentiation, survival, and homeostasis. Cytokines that can be administered to the subject receiving the BAFF-R-binding recombinant receptors, cells and/or compositions provided herein include one or more of IL-2, IL-4, IL-7, IL-9, IL-15, IL-18, and IL-21. Cytokines that can be administered to the subject receiving the BAFF-R-binding recombinant receptors, CD19-binding recombinant receptors, BAFF-R- and CD19-binding recombinant receptors, cells and/or compositions provided herein include one or more of IL-2, IL-4, IL-7, IL-9, IL-15, IL-18, and IL-21. In some embodiments, the cytokine administered is IL-7, IL-15, or IL-21, or a combination thereof. In some embodiments, administration of the cytokine to the may improve certain aspects such as response or anti-tumor activity of the administered cells, e.g., CAR-expressing cells.

Cytokine may refer to proteins released by one cell population that act on another cell as intercellular mediators. Examples of such cytokines are lymphokines, monokines, and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-alpha and -beta; mullerian-inhibiting substance; mouse gonadotropin-associated peptide; inhibin; activin; vascular endothelial growth factor; integrin; thrombopoietin (TPO); nerve growth factors such as NGF-beta; platelet-growth factor; transforming growth factors (TGFs) such as TGF-alpha and TGF-beta; insulin-like growth factor-I and -II; erythropoietin (EPO); osteoinductive factors; interferons such as interferon-alpha, beta, and -gamma; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1alpha, IL-2, IL-3, IL-4, IL-5, IL-6, IL-7, IL-8, IL-9, IL-10, IL-11, IL-12; IL-15, a tumor necrosis factor such as TNF-alpha or TNF-beta; and other polypeptide factors including LIF and kit ligand (KL). As used herein, the term cytokine includes proteins from natural sources or from recombinant cell culture, and biologically active equivalents of the native sequence cytokines. For example, the immunomodulatory agent is a cytokine and the cytokine is IL-4, TNF-α, GM-CSF or IL-2.

In some embodiments, the additional agent includes an interleukin-15 (IL-15) polypeptide, an interleukin-15 receptor alpha (IL-15Rα) polypeptide, or combination thereof, e.g., hetIL-15 (Admune Therapeutics, LLC). hetIL-15 is a heterodimeric non-covalent complex of IL-15 and IL-15Rα. hetIL-15 is described in, e.g., U.S. Pat. No. 8,124,084, U.S. 2012/0177598, U.S. 2009/0082299, U.S. 2012/0141413, and U.S. 2011/0081311. In some embodiments, the immunomodulatory agent can contain one or more cytokines. For example, the interleukin can include leukocyte interleukin injection (Multikine), which is a combination of natural cytokines. In some embodiments, the immunomodulatory agent is a Toll-like receptor (TLR) agonist, an adjuvant or a cytokine.

In some embodiments, the additional agent is an agent that ameliorates or neutralizes one or more toxicities or side effects associated with the cell therapy. In some embodiments, the additional agent is selected from among a steroid (e.g., corticosteroid), an inhibitor of TNFα, and an inhibitor of IL-6. An example of a TNFα inhibitor is an anti-TNFα antibody molecule such as, infliximab, adalimumab, certolizumab pegol, and golimumab. Another example of a TNFα inhibitor is a fusion protein such as entanercept. Small molecule inhibitors of TNFα include, but are not limited to, xanthine derivatives (e.g. pentoxifylline) and bupropion. An example of an IL-6 inhibitor is an anti-IL-6 antibody molecule such as tocilizumab, sarilumab, elsilimomab, CNTO 328, ALD518/BMS-945429, CNTO 136, CPSI-2364, CDP6038, VX30, ARGX-109, FE301, and FM101. In some embodiments, the anti-IL-6 antibody molecule is tocilizumab. In some embodiments, the additional agent is an IL-1R inhibitor, such as anakinra.

In some embodiments, the additional agent is a modulator of adenosine levels and/or an adenosine pathway component. Adenosine can function as an immunomodulatory agent in the body. For example, adenosine and some adenosine analogs that non-selectively activate adenosine receptor subtypes decrease neutrophil production of inflammatory oxidative products (Cronstein et al., Ann. N.Y. Acad. Sci. 451:291, 1985; Roberts et al., Biochem. J., 227:669, 1985; Schrier et al., J. Immunol. 137:3284, 1986; Cronstein et al., Clinical Immunol. Immunopath. 42:76, 1987). In some cases, concentration of extracellular adenosine or adenosine analogs can increase in specific environments, e.g., tumor microenvironment (TME). In some cases, adenosine or adenosine analog signaling depends on hypoxia or factors involved in hypoxia or its regulation, e.g., hypoxia inducible factor (HIF). In some embodiments, increase in adenosine signaling can increase in intracellular CAMP and cAMP-dependent protein kinase that results in inhibition of proinflammatory cytokine production, and can lead to the synthesis of immunosuppressive molecules and development of Tregs (Sitkovsky et al., Cancer Immunol Res (2014) 2(7): 598-605). In some embodiments, the additional agent can reduce or reverse immunosuppressive effects of adenosine, adenosine analogs and/or adenosine signaling. In some embodiments, the additional agent can reduce or reverse hypoxia-driven A2-adenosinergic T cell immunosuppression. In some embodiments, the additional agent is selected from among antagonists of adenosine receptors, extracellular adenosine-degrading agents, inhibitors of adenosine generation by CD39/CD73 ectoenzymes, and inhibitors of hypoxia-HIF-1α signaling. In some embodiments, the additional agent is an adenosine receptor antagonist or agonist.

Inhibition or reduction of extracellular adenosine or the adenosine receptor by virtue of an inhibitor of extracellular adenosine (such as an agent that prevents the formation of, degrades, renders inactive, and/or decreases extracellular adenosine), and/or an adenosine receptor inhibitor (such as an adenosine receptor antagonist) can enhance immune response, such as a macrophage, neutrophil, granulocyte, dendritic cell, T- and/or B cell-mediated response. In addition, inhibitors of the Gs protein mediated cAMP dependent intracellular pathway and inhibitors of the adenosine receptor-triggered Gi protein mediated intracellular pathways, can also increase acute and chronic inflammation.

In some embodiments, the additional agent is an adenosine receptor antagonist or agonist, e.g., an antagonist or agonist of one or more of the adenosine receptors A2a, A2b, A1, and A3. A1 and A3 inhibit, and A2a and A2b stimulate, respectively, adenylate cyclase activity. Certain adenosine receptors, such as A2a, A2b, and A3, can suppress or reduce the immune response during inflammation. Thus, antagonizing immunosuppressive adenosine receptors can augment, boost or enhance immune response, e.g., immune response from administered cells, e.g., CAR-expressing T cells. In some embodiments, the additional agent inhibits the production of extracellular adenosine and adenosine-triggered signaling through adenosine receptors. For example, enhancement of an immune response, local tissue inflammation, and targeted tissue destruction can be enhanced by inhibiting or reducing the adenosine-producing local tissue hypoxia; by degrading (or rendering inactive) accumulated extracellular adenosine; by preventing or decreasing expression of adenosine receptors on immune cells; and/or by inhibiting/antagonizing signaling by adenosine ligands through adenosine receptors.

An antagonist is any substance that tends to nullify the action of another, as an agent that binds to a cell receptor without eliciting a biological response. In some embodiments, the antagonist is a chemical compound that is an antagonist for an adenosine receptor, such as the A2a, A2b, or A3 receptor. In some embodiments, the antagonist is a peptide, or a pepidomimetic, that binds the adenosine receptor but does not trigger a Gi protein dependent intracellular pathway. Exemplary antagonists are described in U.S. Pat. Nos. 5,565,566; 5,545,627, 5,981,524; 5,861,405; 6,066,642; 6,326,390; 5,670,501; 6,117,998; 6,232,297; 5,786,360; 5,424,297; 6,313,131, 5,504,090; and 6,322,771.

In some embodiments, the additional agent is an A2 receptor (A2R) antagonist, such as an A2a antagonist. Exemplary A2R antagonists include KW6002 (istradefyline), SCH58261, caffeine, paraxanthine, 3,7-dimethyl-1-propargylxanthine (DMPX), 8-(m-chlorostyryl) caffeine (CSC), MSX-2, MSX-3, MSX-4, CGS-15943, ZM-241385, SCH-442416, preladenant, vipadenant (BII014), V2006, ST-1535, SYN-115, PSB-1115, ZM241365, FSPTP, and an inhibitory nucleic acid targeting A2R expression, e.g., siRNA or shRNA, or any antibodies or antigen-binding fragment thereof that targets an A2R. In some embodiments, the additional agent is an A2R antagonist described in, e.g., Ohta et al., Proc Natl Acad Sci USA (2006) 103:13132-13137; Jin et al., Cancer Res. (2010) 70(6): 2245-2255; Leone et al., Computational and Structural Biotechnology Journal (2015) 13:265-272; Beavis et al., Proc Natl Acad Sci USA (2013) 110:14711-14716; and Pinna, A., Expert Opin Investig Drugs (2009) 18:1619-1631; Sitkovsky et al., Cancer Immunol Res (2014) 2(7): 598-605; U.S. Pat. Nos. 8,080,554; 8,716,301; US20140056922; WO2008/147482; U.S. Pat. No. 8,883,500; US20140377240; WO02/055083; U.S. Pat. Nos. 7,141,575; 7,405,219; 8,883,500; 8,450,329 and 8,987,279).

In some embodiments, the antagonist is an antisense molecule, inhibitory nucleic acid molecule (e.g., small inhibitory RNA (siRNA)) or catalytic nucleic acid molecule (e.g. a ribozyme) that specifically binds mRNA encoding an adenosine receptor. In some embodiments, the antisense molecule, inhibitory nucleic acid molecule or catalytic nucleic acid molecule binds nucleic acids encoding A2a, A2b, or A3. In some embodiments, an antisense molecule, inhibitory nucleic acid molecule or catalytic nucleic acid targets biochemical pathways downstream of the adenosine receptor. For example, the antisense molecule or catalytic nucleic acid can inhibit an enzyme involved in the Gs protein- or Gi protein-dependent intracellular pathway. In some embodiments, the additional agent includes dominant negative mutant form of an adenosine receptor, such as A2a, A2b, or A3.

In some embodiments, the additional agent that inhibits extracellular adenosine includes agents that render extracellular adenosine non-functional (or decrease such function), such as a substance that modifies the structure of adenosine to inhibit the ability of adenosine to signal through adenosine receptors. In some embodiments, the additional agent is an extracellular adenosine-generating or adenosine-degrading enzyme, a modified form thereof or a modulator thereof. For example, in some embodiments, the additional agent is an enzyme (e.g. adenosine deaminase) or another catalytic molecule that selectively binds and destroys the adenosine, thereby abolishing or significantly decreasing the ability of endogenously formed adenosine to signal through adenosine receptors and terminate inflammation.

In some embodiments, the additional agent is an adenosine deaminase (ADA) or a modified form thereof, e.g., recombinant ADA and/or polyethylene glycol-modified ADA (ADA-PEG), which can inhibit local tissue accumulation of extracellular adenosine. ADA-PEG has been used in treatment of patients with ADA SCID (Hershfield (1995) Hum Mutat. 5:107). In some embodiments, an agent that inhibits extracellular adenosine includes agents that prevent or decrease formation of extracellular adenosine, and/or prevent or decrease the accumulation of extracellular adenosine, thereby abolishing, or substantially decreasing, the immunosuppressive effects of adenosine. In some embodiments, the additional agent specifically inhibits enzymes and proteins that are involved in regulation of synthesis and/or secretion of pro-inflammatory molecules, including modulators of nuclear transcription factors. Suppression of adenosine receptor expression or expression of the Gs protein- or Gi protein-dependent intracellular pathway, or the cAMP dependent intracellular pathway, can result in an increase/enhancement of immune response.

In some embodiments, the additional agent can target ectoenzymes that generate or produce extracellular adenosine. In some embodiments, the additional agent targets CD39 and CD73 ectoenzymes, which function in tandem to generate extracellular adenosine. CD39 (also called ectonucleoside triphosphate diphosphohydrolase) converts extracellular ATP (or ADP) to 5′AMP. Subsequently, CD73 (also called 5′nucleotidase) converts 5′AMP to adenosine. The activity of CD39 is reversible by the actions of NDP kinase and adenylate kinase, whereas the activity of CD73 is irreversible. CD39 and CD73 are expressed on tumor stromal cells, including endothelial cells and Tregs, and also on many cancer cells. For example, the expression of CD39 and CD73 on endothelial cells is increased under the hypoxic conditions of the tumor microenvironment. Tumor hypoxia can result from inadequate blood supply and disorganized tumor vasculature, impairing delivery of oxygen (Carroll and Ashcroft (2005), Expert. Rev. Mol. Med. 7(6): 1-16). Hypoxia also inhibits adenylate kinase (AK), which converts adenosine to AMP, leading to very high extracellular adenosine concentration. Thus, adenosine is released at high concentrations in response to hypoxia, which is a condition that frequently occurs the tumor microenvironment (TME), in or around solid tumors. In some embodiments, the additional agent is one or more of anti-CD39 antibody or antigen binding fragment thereof, anti-CD73 antibody or antigen binding fragment thereof, e.g., MEDI9447 or TY/23, α-β-methylene-adenosine diphosphate (ADP), ARL 67156, POM-3, IPH52 (see, e.g., Allard et al. Clin Cancer Res (2013) 19(20): 5626-5635; Hausler et al., Am J Transl Res (2014) 6(2): 129-139; Zhang, B., Cancer Res. (2010) 70(16): 6407-6411).

In some embodiments, the additional agent is an inhibitor of hypoxia inducible factor 1 alpha (HIF-1α) signaling. Exemplary inhibitors of HIF-1α include digoxin, acriflavine, sirtuin-7 and ganetespib.

In some embodiments, the additional agent includes a protein tyrosine phosphatase inhibitor, e.g., a protein tyrosine phosphatase inhibitor described herein. In some embodiments, the protein tyrosine phosphatase inhibitor is an SHP-1 inhibitor, e.g., an SHP-1 inhibitor described herein, such as, e.g., sodium stibogluconate. In some embodiments, the protein tyrosine phosphatase inhibitor is an SHP-2 inhibitor, e.g., an SHP-2 inhibitor described herein.

In some embodiments, the additional agent is a kinase inhibitor. Kinase inhibitors, such as a CDK4 kinase inhibitor, a BTK kinase inhibitor, a MNK kinase inhibitor, or a DGK kinase inhibitor, can regulate the constitutively active survival pathways that exist in tumor cells and/or modulate the function of immune cells. In some embodiments, the kinase inhibitor is a Bruton's tyrosine kinase (BTK) inhibitor, e.g., ibrutinib. In some embodiments, the kinase inhibitor is a phosphatidylinositol-4,5-bisphosphate 3-kinase (PI3K) inhibitor. In some embodiments, the kinase inhibitor is a CDK4 inhibitor, e.g., a CDK4/6 inhibitor. In some embodiments, the kinase inhibitor is an mTOR inhibitor, such as, e.g., rapamycin, a rapamycin analog, OSI-027. The mTOR inhibitor can be, e.g., an mTORC1 inhibitor and/or an mTORC2 inhibitor, e.g., an mTORC1 inhibitor and/or mTORC2 inhibitor. In some embodiments, the kinase inhibitor is an MNK inhibitor, or a dual PI3K/mTOR inhibitor. In some embodiments, other exemplary kinase inhibitors include the AKT inhibitor perifosine, the mTOR inhibitor temsirolimus, the Src kinase inhibitors dasatinib and fostamatinib, the JAK2 inhibitors pacritinib and ruxolitinib, the PKCβ inhibitors enzastaurin and bryostatin, and the AAK inhibitor alisertib.

In some embodiments, the kinase inhibitor is a BTK inhibitor selected from ibrutinib (PCI-32765); GDC-0834; RN-486; CGI-560; CGI-1764; HM-71224; CC-292; ONO-4059; CNX-774; and LFM-A13. In some embodiments, the BTK inhibitor does not reduce or inhibit the kinase activity of interleukin-2-inducible kinase (ITK), and is selected from GDC-0834; RN-486; CGI-560; CGI-1764; HM-71224; CC-292; ONO-4059; CNX-774; and LFM-A13.

In some embodiments, the kinase inhibitor is a BTK inhibitor, e.g., ibrutinib (1-[(3R)-3-[4-Amino-3-(4-phenoxyphenyl)-1H-pyrazolo[3,4-d]pyrimidin-1-yl]piperidin-1-yl]prop-2-en-1-one; also known as PCI-32765). In some embodiments, the kinase inhibitor is a BTK inhibitor, e.g., ibrutinib (PCI-32765), and the ibrutinib is administered at a dose of about 250 mg, 300 mg, 350 mg, 400 mg, 420 mg, 440 mg, 460 mg, 480 mg, 500 mg, 520 mg, 540 mg, 560 mg, 580 mg, 600 mg (e.g., 250 mg, 420 mg or 560 mg) daily for a period of time, e.g., daily for 21 day cycle, or daily for 28 day cycle. In some embodiments, 1, 2, 3, 4, 5, 6, 7, 8, 9, 10, 11, 12 or more cycles of ibrutinib are administered. In some embodiments, the BTK inhibitor is a BTK inhibitor described in International Application WO 2015/079417.

In some embodiments, the kinase inhibitor is a PI3K inhibitor. PI3K is central to the PI3K/Akt/mTOR pathway involved in cell cycle regulation and lymphoma survival. Exemplary PI3K inhibitor includes idelalisib (PI3Kδ inhibitor). In some embodiments, the additional agent is idelalisib and rituximab.

In some embodiments, the additional agent is an inhibitor of mammalian target of rapamycin (mTOR). In some embodiments, the kinase inhibitor is an mTOR inhibitor selected from temsirolimus; ridaforolimus (also known as AP23573 and MK8669); everolimus (RAD001); rapamycin (AY22989); simapimod; AZD8055; PF04691502; SF1126; and XL765. In some embodiments, the additional agent is an inhibitor of mitogen-activated protein kinase (MAPK), such as vemurafenib, dabrafenib, and trametinib.

In some embodiments, the additional agent is an agent that regulates pro- or anti-apoptotic proteins. In some embodiments, the additional agent includes a B-cell lymphoma 2 (BCL-2) inhibitor (e.g., venetoclax, also called ABT-199 or GDC-0199; or ABT-737). Venetoclax is a small molecule (4-(4-{[2-(4-Chlorophenyl)-4,4-dimethyl-1-cyclohexen-1-yl]methyl}-1-piperazinyl)-N-({3-nitro-4-[(tetrahydro-2H-pyran-4-ylmethyl)amino]phenyl}sulfonyl)-2-(1H-pyrrolo[2,3-b]pyridin-5-yloxy)benzamide) that inhibits the anti-apoptotic protein, BCL-2. Other agents that modulate pro- or anti-apoptotic protein include BCL-2 inhibitor ABT-737, navitoclax (ABT-263); Mcl-1 siRNA or Mcl-1 inhibitor retinoid N-(4-hydroxyphenyl) retinamide (4-HPR) for maximal efficacy. In some embodiments, the additional agent provides a pro-apoptotic stimuli, such as recombinant tumor necrosis factor-related apoptosis-inducing ligand (TRAIL), which can activate the apoptosis pathway by binding to TRAIL death receptors DR-4 and DR-5 on tumor cell surface, or TRAIL-R2 agonistic antibodies.

In some embodiments, the additional agent includes an indoleamine 2,3-dioxygenase (IDO) inhibitor. IDO is an enzyme that catalyzes the degradation of the amino acid, L-tryptophan, to kynurenine. Many cancers overexpress IDO, e.g., prostatic, colorectal, pancreatic, cervical, gastric, ovarian, head, and lung cancer. Plasmacytoid dendritic cells (pDCs), macrophages, and dendritic cells (DCs) can express IDO. In some aspects, a decrease in L-tryptophan (e.g., catalyzed by IDO) results in an immunosuppressive milieu by inducing T-cell anergy and apoptosis. Thus, in some aspects, an IDO inhibitor can enhance the efficacy of the BAFF-R-binding recombinant receptors, cells and/or compositions described herein, e.g., by decreasing the suppression or death of the administered CAR-expressing cell. Exemplary inhibitors of IDO include but are not limited to 1-methyl-tryptophan, indoximod (New Link Genetics) (see, e.g., Clinical Trial Identifier Nos. NCT01191216; NCT01792050), and INCB024360 (Incyte Corp.) (see, e.g., Clinical Trial Identifier Nos. NCT01604889; NCT01685255).

In some embodiments, the additional agent includes a cytotoxic agent, e.g., CPX-351 (Celator Pharmaceuticals), cytarabine, daunorubicin, vosaroxin (Sunesis Pharmaceuticals), sapacitabine (Cyclacel Pharmaceuticals), idarubicin, or mitoxantrone. In some embodiments, the additional agent includes a hypomethylating agent, e.g., a DNA methyltransferase inhibitor, e.g., azacitidine or decitabine.

In another embodiment, the additional therapy is transplantation, e.g., an allogeneic stem cell transplant.

In some embodiments, the additional therapy is a lymphodepleting therapy. Lymphodepleting chemotherapy is thought to improve engraftment and activity of recombinant receptor-expressing cells, such as CAR T cells. In some embodiments, lymphodepleting chemotherapy may enhance adoptively transferred tumor-specific T cells to proliferate in vivo through homeostatic proliferation (Grossman 2004, Stachel 2004). In some embodiments, chemotherapy may reduce or eliminate CD4+CD25+ regulatory T cells, which can suppress the function of tumor-targeted adoptively transferred T cells (Turk 2004). In some embodiments, lymphodepleting chemotherapy prior to adoptive T-cell therapy may enhance the expression of stromal cell-derived factor 1 (SDF-1) in the bone marrow, enhancing the homing of modified T cells to the primary tumor site through binding of SDF-1 with CXCR-4 expressed on the T-cell surface (Pinthus 2004). In some embodiments, lymphodepleting chemotherapy may further reduce the subject's tumor burden and potentially lower the risk and severity of CRS.

In some embodiments, lymphodepletion is performed on a subject, e.g., prior to administering engineered cells, e.g., CAR-expressing cells. In some embodiments, the lymphodepletion comprises administering one or more of melphalan, Cytoxan, cyclophosphamide, and/or fludarabine. In some embodiments, a lymphodepleting chemotherapy is administered to the subject prior to, concurrently with, or after administration (e.g., infusion) of engineered cells, e.g., CAR-expressing cells. In an example, the lymphodepleting chemotherapy is administered to the subject prior to administration of engineered cells, e.g., CAR-expressing cells. In some embodiments the lymphodepleting chemotherapy is administered 1 to 10 days prior to administration of engineered cells, such as 1, 2, 3, 4, 5, 6, 7, 8, 9, or 10 days prior to the initiation of administration of engineered cells, or at least 2 days prior, such as at least 3, 4, 5, 6, or 7 days prior, to the initiation of administration of engineered cell. In some embodiments, the subject is administered a preconditioning agent no more than 7 days prior, such as no more than 6, 5, 4, 3, or 2 days prior, to the initiation of administration of engineered cell. The number of days after lymphodepleting chemotherapy that the engineered ells are administered can be determined based on clinical or logistical circumstances. In some examples, dose adjustments or other changes to the lymphodepleting chemotherapy regimen can implemented due to a subject's health, such as the subject's underlying organ function, as determined by the treating physician.

In some embodiments, lymphodepleting chemotherapy comprises administration of a lymphodepleting agent, such as cyclophosphamide, fludarabine, or combinations thereof. In some embodiments, the subject is administered cyclophosphamide at a dose between or between about 20 mg/kg and 100 mg/kg body weight of the subject, such as between or between about 40 mg/kg and 80 mg/kg. In some aspects, the subject is administered about 60 mg/kg of cyclophosphamide. In some embodiments, the cyclophosphamide is administered once daily for one or two days. In some embodiments, where the lymphodepleting agent comprises cyclophosphamide, the subject is administered cyclophosphamide at a dose between or between about 100 mg/m² and 500 mg/m² body surface area of the subject, such as between or between about 200 mg/m² and 400 mg/m², or 250 mg/m² and 350 mg/m², inclusive. In some instances, the subject is administered about 300 mg/m² of cyclophosphamide. In some embodiments, the cyclophosphamide can be administered in a single dose or can be administered in a plurality of doses, such as given daily, every other day or every three days. In some embodiments, cyclophosphamide is administered daily, such as for 1-5 days, for example, for 2 to 4 days. In some instances, the subject is administered about 300 mg/m² of cyclophosphamide, daily for 3 days, prior to initiation of the cell therapy.

In some embodiments, where the lymphodepleting agent comprises fludarabine, the subject is administered fludarabine at a dose between or between about 1 mg/m² and 100 mg/m² body surface area of the subject, such as between or between about 10 mg/m² and 75 mg/m², 15 mg/m² and 50 mg/m², 20 mg/m² and 40 mg/m², or 24 mg/m² and 35 mg/m², inclusive. In some instances, the subject is administered about 30 mg/m² of fludarabine. In some embodiments, the fludarabine can be administered in a single dose or can be administered in a plurality of doses, such as given daily, every other day or every three days. In some embodiments, fludarabine is administered daily, such as for 1-5 days, for example, for 2 to 4 days. In some instances, the subject is administered about 30 mg/m² of fludarabine, daily for 3 days, prior to initiation of the cell therapy.

In some embodiments, the lymphodepleting agent comprises a combination of agents, such as a combination of cyclophosphamide and fludarabine. Thus, the combination of agents may include cyclophosphamide at any dose or administration schedule, such as those described above, and fludarabine at any dose or administration schedule, such as those described above. For example, in some aspects, the subject is administered fludarabine at or about 30 mg/m², daily, and cyclophosphamide at or about 300 mg/m², daily, for 3 days.

In some embodiments, antiemetic therapy, except dexamethasone or other steroids, may be given prior to lymphodepleting chemotherapy. In some embodiments, Mesna may be used for subjects with a history of hemorrhagic cystitis.

In some embodiments, the additional agent is an oncolytic virus. In some embodiments, oncolytic viruses are capable of selectively replicating in and triggering the death of or slowing the growth of a cancer cell. In some cases, oncolytic viruses have no effect or a minimal effect on non-cancer cells. An oncolytic virus includes but is not limited to an oncolytic adenovirus, oncolytic Herpes Simplex Viruses, oncolytic retrovirus, oncolytic parvovirus, oncolytic vaccinia virus, oncolytic Sinbis virus, oncolytic influenza virus, or oncolytic RNA virus (e.g., oncolytic reovirus, oncolytic Newcastle Disease Virus (NDV), oncolytic measles virus, or oncolytic vesicular stomatitis virus (VSV)).

Other exemplary combination therapy, treatment and/or agents include anti-allergenic agents, anti-emetics, analgesics and adjunct therapies. In some embodiments, the additional agent includes cytoprotective agents, such as neuroprotectants, free-radical scavengers, cardioprotectors, anthracycline extravasation neutralizers and nutrients.

In some embodiments, an antibody used as an additional agent is conjugated or otherwise bound to a therapeutic agent, e.g., a chemotherapeutic agent (e.g., Cytoxan, fludarabine, histone deacetylase inhibitor, demethylating agent, peptide vaccine, anti-tumor antibiotic, tyrosine kinase inhibitor, alkylating agent, anti-microtubule or anti-mitotic agent), anti-allergic agent, anti-nausea agent (or anti-emetic), pain reliever, or cytoprotective agent described herein. In some embodiments, the additional agent is an antibody-drug conjugate.

In some embodiments, the additional agent can modulate, inhibit or stimulate particular factors at the DNA, RNA or protein levels, such as to enhance or boost certain aspects. In some embodiments, the additional agent can modulate the factors at the nucleic acid level, e.g., DNA or RNA, within the administered cells, e.g., cells engineered to express recombinant receptors, e.g., CAR. In some embodiments, an inhibitory nucleic acid, e.g., an inhibitory nucleic acid, e.g., a dsRNA, e.g., an siRNA or shRNA, or a clustered regularly interspaced short palindromic repeats (CRISPR), a transcription-activator like effector nuclease (TALEN), or a zinc finger endonuclease (ZFN), can be used to inhibit expression of an inhibitory molecule in the engineered cell, e.g., CAR-expressing cell. In some embodiments the inhibitor is an shRNA. In some embodiments, the inhibitory molecule is inhibited within the engineered cell, e.g., CAR-expressing cell. In some embodiments, a nucleic acid molecule that encodes a dsRNA molecule that inhibits expression of the molecule that modulates or regulates, e.g., inhibits, T-cell function is operably linked to a promoter, e.g., a HI- or a U6-derived promoter such that the dsRNA molecule that inhibits expression of the inhibitory molecule is expressed within the engineered cell, e.g., CAR-expressing cell. See, e.g., Brummelkamp T R, et al. (2002) Science 296:550-553; Miyagishi M, et al. (2002) Nat. Biotechnol. 19:497-500.

In some embodiments, the additional agent is capable of disrupting the gene encoding an inhibitory molecule, such as any immune checkpoint inhibitors described herein. In some embodiments, disruption is by deletion, e.g., deletion of an entire gene, exon, or region, and/or replacement with an exogenous sequence, and/or by mutation, e.g., frameshift or missense mutation, within the gene, typically within an exon of the gene. In some embodiments, the disruption results in a premature stop codon being incorporated into the gene, such that the inhibitory molecule is not expressed or is not expressed in a form that is capable of being expressed on the cells surface and/or capable of mediating cell signaling. The disruption is generally carried out at the DNA level. The disruption generally is permanent, irreversible, or not transient.

In some aspects, the disruption is carried out by gene editing, such as using a DNA binding protein or DNA-binding nucleic acid, which specifically binds to or hybridizes to the gene at a region targeted for disruption. In some aspects, the protein or nucleic acid is coupled to or complexed with a nuclease, such as in a chimeric or fusion protein. For example, in some embodiments, the disruption is effected using a fusion comprising a DNA-targeting protein and a nuclease, such as a Zinc Finger Nuclease (ZFN) or TAL-effector nuclease (TALEN), or an RNA-guided nuclease such as a clustered regularly interspersed short palindromic nucleic acid (CRISPR)-Cas system, such as CRISPR-Cas9 system, specific for the gene being disrupted. In some embodiments, methods of producing or generating genetically engineered cells, e.g., CAR-expressing cells, include introducing into a population of cells nucleic acid molecules encoding a genetically engineered antigen receptor (e.g. CAR) and nucleic acid molecules encoding an agent targeting an inhibitory molecule that is a gene editing nuclease, such as a fusion of a DNA-targeting protein and a nuclease such as a ZFN or a TALEN, or an RNA-guided nuclease such as of the CRISPR-Cas9 system, specific for an inhibitory molecule.

Any of the additional agents described herein can be prepared and administered as combination therapy with the BAFF-R- and CD19-binding recombinant receptor (e.g., bispecific chimeric antigen receptor) and/or engineered cells expressing said molecules (e.g., recombinant receptor) described herein, such as in pharmaceutical compositions comprising one or more agents of the combination therapy and a pharmaceutically acceptable carrier, such as any described herein. In some embodiments, the BAFF-R- and CD19-binding recombinant receptor (e.g., bispecific chimeric antigen receptor), engineered cells expressing said molecules (e.g., recombinant receptor), plurality of engineered cells expressing said molecules (e.g., recombinant receptor) can be administered simultaneously, concurrently or sequentially, in any order with the additional agents, therapy or treatment, wherein such administration provides therapeutically effective levels each of the agents in the body of the subject. In some embodiments, the additional agent can be co-administered with the BAFF-R- and CD19-binding recombinant receptors, cells and/or compositions described herein, for example, as part of the same pharmaceutical composition or using the same method of delivery. In some embodiments, the additional agent is administered simultaneously with the BAFF-R- and CD19-binding recombinant receptors, cells and/or compositions described herein, but in separate compositions. In some embodiments, the additional agent is an additional engineered cell, e.g., cell engineered to express a different recombinant receptor, and is administered in the same composition or in a separate composition. In some embodiments, the additional agent is incubated with the engineered cell, e.g., CAR-expressing cells, prior to administration of the cells.

In some examples, the one or more additional agents are administered subsequent to or prior to the administration of the BAFF-R- and CD19-binding recombinant receptors, cells and/or compositions described herein, separated by a selected time period. In some examples, the time period is 1 day, 2 days, 3 days, 4 days, 5 days, 6 days, 1 week, 2 weeks, 3 weeks, 1 month, 2 months, or 3 months. In some examples, the one or more additional agents are administered multiple times and/or the BAFF-R- and CD19-binding recombinant receptors, cells and/or compositions described herein, is administered multiple times. For example, in some embodiments, the additional agent is administered prior to the BAFF-R- and CD19-binding recombinant receptors, cells and/or compositions described herein, e.g., two weeks, 12 days, 10 days, 8 days, one week, 6 days, 5 days, 4 days, 3 days, 2 days or 1 day before the administration.

The dose of the additional agent can be any therapeutically effective amount, e.g., any dose amount described herein, and the appropriate dosage of the additional agent may depend on the type of disease to be treated, the type, dose and/or frequency of the recombinant receptor, cell and/or composition administered, the severity and course of the disease, whether the recombinant receptor, cell and/or composition is administered for preventive or therapeutic purposes, previous therapy, the patient's clinical history and response to the recombinant receptor, cell and/or composition, and the discretion of the attending physician. The recombinant receptor, cell and/or composition and/or the additional agent and/or therapy can be administered to the patient at one time, repeated or administered over a series of treatments.

### VI. ARTICLES OF MANUFACTURE OR KITS

Also provided are articles of manufacture or kits containing the provided recombinant receptors (e.g., CARs), genetically engineered cells, and/or compositions comprising the same. The articles of manufacture may include a container and a label or package insert on or associated with the container. Suitable containers include, for example, bottles, vials, syringes, test tubes, IV solution bags, etc. The containers may be formed from a variety of materials such as glass or plastic. In some embodiments, the container has a sterile access port. Exemplary containers include an intravenous solution bags, vials, including those with stoppers pierceable by a needle for injection. The article of manufacture or kit may further include a package insert indicating that the compositions can be used to treat a particular condition such as a condition described herein (e.g., a cancer).

Alternatively, or additionally, the article of manufacture or kit may further include another or the same container comprising a pharmaceutically-acceptable buffer. It may further include other materials such as other buffers, diluents, filters, needles, and/or syringes.

The label or package insert may indicate that the composition is used for treating the BAFF-R-expressing or BAFF-R-associated disease, disorder or condition in an individual. The label or package insert may indicate that the composition is used for treating the CD19-expressing or CD19-associated disease, disorder or condition in an individual. The label or a package insert, which is on or associated with the container, may indicate directions for reconstitution and/or use of the formulation. The label or package insert may further indicate that the formulation is useful or intended for subcutaneous, intravenous, or other modes of administration for treating or preventing a BAFF-R-expressing or BAFF-R-associated disease, disorder or condition in an individual. The label or package insert may further indicate that the formulation is useful or intended for subcutaneous, intravenous, or other modes of administration for treating or preventing a CD19-expressing or CD19-associated disease, disorder or condition in an individual.

The container in some embodiments holds a composition which is by itself or combined with another composition effective for treating, preventing and/or diagnosing the condition. The article of manufacture or kit may include (a) a first container with a composition contained therein (i.e., first medicament), wherein the composition includes the CAR; and (b) a second container with a composition contained therein (i.e., second medicament), wherein the composition includes a further agent, such as a cytotoxic or otherwise therapeutic agent, and which article or kit further comprises instructions on the label or package insert for treating the subject with the second medicament, in an effective amount.

### VII. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, reference to a “corresponding form” of an antibody means that when comparing a property or activity of two antibodies, the property is compared using the same form of the antibody. For example, if it is stated that an antibody has greater activity compared to the activity of the corresponding form of a first antibody, that means that a particular form, such as an scFv of that antibody, has greater activity compared to the scFv form of the first antibody.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

The terms “full length antibody,” “intact antibody,” and “whole antibody” are used herein interchangeably to refer to an antibody having a structure substantially similar to a native antibody structure or having heavy chains that contain an Fc region as defined herein.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

The terms “polypeptide” and “protein” are used interchangeably to refer to a polymer of amino acid residues, and are not limited to a minimum length. Polypeptides, including the antibodies and antibody chains and other peptides, e.g., linkers, may include amino acid residues including natural and/or non-natural amino acid residues. The terms also include post-expression modifications of the polypeptide, for example, glycosylation, sialylation, acetylation, phosphorylation, and the like. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

As used herein, “percent (%) amino acid sequence identity” and “percent identity” and “sequence identity” when used with respect to an amino acid sequence (reference polypeptide sequence) is defined as the percentage of amino acid residues in a candidate sequence (e.g., the subject antibody or fragment) that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

An amino acid substitution may include replacement of one amino acid in a polypeptide with another amino acid. Amino acid substitutions may be introduced into a binding molecule, e.g., antibody, of interest and the products screened for a desired activity, e.g., retained/improved antigen binding, or decreased immunogenicity.

Amino acids generally can be grouped according to the following common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative amino acid substitutions will involve exchanging a member of one of these classes for another class.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The term “package insert” is used to refer to instructions customarily included in commercial packages of therapeutic products, that contain information about the indications, usage, dosage, administration, combination therapy, contraindications and/or warnings concerning the use of such therapeutic products.

As used herein, the singular forms “a,” “an,” and “the” include plural referents unless the context clearly dictates otherwise. For example, “a” or “an” means “at least one” or “one or more.” It is understood that aspects, embodiments, and variations described herein include “comprising,” “consisting,” and/or “consisting essentially of” aspects, embodiments and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X”.

As used herein, a “composition” refers to any mixture of two or more products, substances, or compounds, including cells. It may be a solution, a suspension, liquid, powder, a paste, aqueous, non-aqueous or any combination thereof.

As used herein, a statement that a cell or population of cells is “positive” for a particular marker refers to the detectable presence on or in the cell of a particular marker, typically a surface marker. When referring to a surface marker, the term refers to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control under otherwise identical conditions and/or at a level substantially similar to that for cell known to be positive for the marker, and/or at a level substantially higher than that for a cell known to be negative for the marker.

As used herein, a statement that a cell or population of cells is “negative” for a particular marker refers to the absence of substantial detectable presence on or in the cell of a particular marker, typically a surface marker. When referring to a surface marker, the term refers to the absence of surface expression as detected by flow cytometry, for example, by staining with an antibody that specifically binds to the marker and detecting said antibody, wherein the staining is not detected by flow cytometry at a level substantially above the staining detected carrying out the same procedure with an isotype-matched control under otherwise identical conditions, and/or at a level substantially lower than that for cell known to be positive for the marker, and/or at a level substantially similar as compared to that for a cell known to be negative for the marker.

As used herein, the recitation of a numerical range for a variable is intended to convey that the invention may be practiced with the variable equal to any of the values within that range. Thus, for a variable which is inherently discrete, the variable can be equal to any integer value within the numerical range, including the end-points of the range. Similarly, for a variable which is inherently continuous, the variable can be equal to any real value within the numerical range, including the end-points of the range. As an example, and without limitation, a variable which is described as having values between 0 and 2 can take the values 0, 1 or 2 if the variable is inherently discrete, and can take the values 0.0, 0.1, 0.01, 0.001, or any other real values >0 and <2 if the variable is inherently continuous.

### VIII. EXEMPLARY EMBODIMENTS

Among the provided embodiments are:

1. A bispecific chimeric antigen receptor (CAR) comprising an extracellular binding domain, a spacer, a transmembrane domain, and an intracellular signaling domain, wherein the extracellular binding domain comprises:
a B-cell activating factor receptor (BAFF-R)-binding domain that binds to BAFF-R comprising a heavy chain variable (V_{H}) region and a light chain variable (V_{L}) region; and
a CD19-binding domain that binds to CD19 comprising a V_{H} region and a V_{L} region, wherein the extracellular binding domain comprises in order from the amino- to carboxy-terminus:
(i) the V_{H} region of the BAFF-R-binding domain, the V_{L} region of the CD19-binding domain, the V_{H} region of the CD19-binding domain, and the V_{L} region of the BAFF-R binding domain;
(ii) the V_{L} region of the BAFF-R-binding domain, the V_{L} region of the CD19-binding domain, the V_{H} region of the CD19-binding domain, and the V_{H} region of the BAFF-R binding domain;
(iii) the V_{H} region of the BAFF-R-binding domain, the V_{H} region of the CD19-binding domain, the V_{L} region of the CD19-binding domain, and the V_{L} region of the BAFF-R binding domain; or
(iv) the V_{L} region of the BAFF-R-binding domain, the V_{H} region of the CD19-binding domain, the V_{L} region of the CD19-binding domain, and the V_{H} region of the BAFF-R binding domain.

2. The bispecific CAR of embodiment 1, wherein the extracellular binding domain comprises in order from amino- to carboxy-terminus: the V_{L} region of the BAFF-R-binding domain, the V_{L} region of the CD19-binding domain, the V_{H} region of the CD19-binding domain, and the V_{H} region of the BAFF-R-binding domain.

3. The bispecific CAR of embodiment 1, wherein the extracellular binding domain comprises in order from amino- to carboxy-terminus: the V_{H} region of the BAFF-R-binding domain, the V_{L} region of the CD19-binding domain, the V_{H} region of the CD19-binding domain, and the V_{L} region of the BAFF-R-binding domain.

4. The bispecific CAR of any one of embodiments 1-3, wherein:
(i) the V_{H} region of the BAFF-R-binding domain comprises a CDR-H1, a CDR-H2 and a CDR-H3 each comprising a sequence that is contained within SEQ ID NO:1, and the V_{L} region of the BAFF-R-binding domain comprises a CDR-L1, a CDR-L2 and a CDR-L3 each comprising a sequence that is contained within SEQ ID NO:2;
(ii) the V_{H} region of the BAFF-R-binding domain comprises a CDR-H1, a CDR-H2 and a CDR-H3 each comprising a sequence that is contained within SEQ ID NO:3, and the V_{L} region of the BAFF-R-binding domain comprises a CDR-L1, a CDR-L2 and a CDR-L3 each having a sequence that is contained within SEQ ID NO:4;
(iii) the V_{H} region of the BAFF-R-binding domain comprises a CDR-H1, a CDR-H2 and a CDR-H3 each having a sequence that is contained within SEQ ID NO:5, and the V_{L} region of the BAFF-R-binding domain comprises a CDR-L1, a CDR-L2 and a CDR-L3 each comprising a sequence that is contained within SEQ ID: NO 6;
(iv) the V_{H} region of the BAFF-R-binding domain comprises a CDR-H1, a CDR-H2 and a CDR-H3 each comprising a sequence that is contained within SEQ ID NO:7, and the V_{L} region of the BAFF-R-binding domain comprises a CDR-L1, a CDR-L2 and a CDR-L3 each comprising a sequence that is contained within SEQ ID: NO 8; or
(v) the V_{H} region of the BAFF-R-binding domain comprises a CDR-H1, a CDR-H2 and a CDR-H3 each comprising a sequence that is contained within SEQ ID NO:9, and the V_{L} region of the BAFF-R-binding domain comprises a CDR-L1, a CDR-L2 and a CDR-L3 each comprising a sequence that is contained within SEQ ID: NO 10.

5. The bispecific CAR of any one of embodiments 1-4, wherein each CDR is defined in accordance with the Kabat definition, the Chothia definition, the combination of the Kabat and the Chothia definition, the AbM definition, or the contact definition.

6. A bispecific CAR comprising an extracellular binding domain, a transmembrane domain, a spacer, and an intracellular signaling domain, wherein the extracellular binding domain comprises:
a B-cell activating factor receptor (BAFF-R)-binding domain that binds to BAFF-R comprising a heavy chain variable (V_{H}) region and a light chain variable (V_{L}) region; and
a CD19-binding domain that binds to CD19 comprising a V_{H} region and a V_{L} region, wherein: (i) the V_{H} region of the BAFF-R-binding domain comprises a CDR-H1, a CDR-H2 and a CDR-H3 each comprising a sequence that is contained within SEQ ID NO:1, and the V_{L} region of the BAFF-R-binding domain comprises a CDR-L1, a CDR-L2 and a CDR-L3 each comprising a sequence that is contained within SEQ ID NO:2;
(ii) the V_{H} region of the BAFF-R-binding domain comprises a CDR-H1, a CDR-H2 and a CDR-H3 each comprising a sequence that is contained within SEQ ID NO:3, and the V_{L} region of the BAFF-R-binding domain comprises a CDR-L1, a CDR-L2 and a CDR-L3 each comprising a sequence that is contained within SEQ ID NO:4;
(iii) the V_{H} region of the BAFF-R-binding domain comprises a CDR-H1, a CDR-H2 and a CDR-H3 each comprising a sequence that is contained within SEQ ID NO:5, and the V_{L} region of the BAFF-R-binding domain comprises CDR-L1, CDR-L2 and CDR-L3 each comprising a sequence that is contained within SEQ ID: NO 6;
(iv) the V_{H} region of the BAFF-R-binding domain comprises a CDR-H1, a CDR-H2 and a CDR-H3 each comprising a sequence that is contained within SEQ ID NO:7, and the V_{L} region of the BAFF-R-binding domain comprises a CDR-L1, a CDR-L2 and a CDR-L3 each comprising a sequence that is contained within SEQ ID: NO 8; or
(v) the V_{H} region of the BAFF-R-binding domain comprises a CDR-H1, a CDR-H2 and a CDR-H3 each comprising a sequence that is contained within SEQ ID NO:9, and the V_{L} region of the BAFF-R-binding domain comprises a CDR-L1, a CDR-L2 and a CDR-L3 each comprising a sequence that is contained within SEQ ID: NO 10.

7. The bispecific CAR of embodiment 6, wherein each CDR is defined in accordance with the Kabat definition, the Chothia definition, the combination of the Kabat and the Chothia definition, the AbM definition, or the contact definition.

8. The bispecific CAR of any one of embodiments 1-7, wherein:
(i) the V_{H} region of the BAFF-R-binding domain comprises CDR-H1, CDR-H2 and CDR-H3 sequences set forth in SEQ ID NOS: 16, 17, and 18, respectively; and the V_{L} region of the BAFF-R-binding domain comprises CDR-L1, CDR-L2 and CDR-L3 sequences set forth in SEQ ID NOS: 19, 20, and 21, respectively;
(ii) the V_{H} region of the BAFF-R-binding domain comprises CDR-H1, CDR-H2 and CDR-H3 sequences set forth in SEQ ID NOS: 22, 23, and 24, respectively; and the V_{L} region of the BAFF-R-binding domain comprises CDR-L1, CDR-L2 and CDR-L3 sequences set forth in SEQ ID NOS: 25, 26, and 27, respectively;
(iii) the V_{H} region of the BAFF-R-binding domain comprises CDR-H1, CDR-H2 and CDR-H3 sequences set forth in SEQ ID NOS: 28, 29, and 30, respectively; and the V_{L} region of the BAFF-R-binding domain comprises CDR-L1, CDR-L2 and CDR-L3 sequences set forth in SEQ ID NOS: 31, 26, and 27, respectively;
(iv) the V_{H} region of the BAFF-R-binding domain comprises CDR-H1, CDR-H2 and CDR-H3 sequences set forth in SEQ ID NOS: 22, 32 and 24, respectively; and the V_{L} region of the BAFF-R-binding domain comprises CDR-L1, CDR-L2 and CDR-L3 sequences set forth in SEQ ID NOS: 33, 26, and 34, respectively; or
(v) the V_{H} region of the BAFF-R-binding domain comprises CDR-H1, CDR-H2 and CDR-H3 sequences set forth in SEQ ID NOS: 35, 36, and 37, respectively; and the V_{L} region of the BAFF-R-binding domain comprises CDR-L1, CDR-L2 and CDR-L3 sequences set forth in SEQ ID NOS: 38, 39, and 40, respectively.

9. A bispecific CAR comprising an extracellular binding domain, a transmembrane domain, a spacer, and an intracellular signaling domain, wherein the extracellular binding domain comprises:
a B-cell activating factor receptor (BAFF-R)-binding domain that binds to BAFF-R comprising a heavy chain variable (V_{H}) region and a light chain variable (V_{L}) region; and
a CD19-binding domain that binds to CD19 comprising a V_{H} region and a V_{L} region, wherein:
(i) the V_{H} region of the BAFF-R-binding domain comprises CDR-H1, CDR-H2 and CDR-H3 sequences set forth in SEQ ID NOS: 16, 17, and 18, respectively; and the V_{L} region of the BAFF-R-binding domain comprises CDR-L1, CDR-L2 and CDR-L3 sequences set forth in SEQ ID NOS: 19, 20, and 21, respectively;
(ii) the V_{H} region of the BAFF-R-binding domain comprises CDR-H1, CDR-H2 and CDR-H3 sequences set forth in SEQ ID NOS: 22, 23, and 24, respectively; and the V_{L} region of the BAFF-R-binding domain comprises CDR-L1, CDR-L2 and CDR-L3 sequences set forth in SEQ ID NOS: 25, 26, and 27, respectively;
(iii) the V_{H} region of the BAFF-R-binding domain comprises CDR-H1, CDR-H2 and CDR-H3 sequences set forth in SEQ ID NOS: 28, 29, and 30, respectively; and the V_{L} region of the BAFF-R-binding domain comprises CDR-L1, CDR-L2 and CDR-L3 sequences set forth in SEQ ID NOS: 31, 26, and 27, respectively;
(iv) the V_{H} region of the BAFF-R-binding domain comprises CDR-H1, CDR-H2 and CDR-H3 sequences set forth in SEQ ID NOS: 22, 32 and 24, respectively; and the V_{L} region of the BAFF-R-binding domain comprises CDR-L1, CDR-L2 and CDR-L3 sequences set forth in SEQ ID NOS: 33, 26, and 34, respectively; or
(v) the V_{H} region of the BAFF-R-binding domain comprises CDR-H1, CDR-H2 and CDR-H3 sequences set forth in SEQ ID NOS: 35, 36, and 37, respectively; and the V_{L} region of the BAFF-R-binding domain comprises CDR-L1, CDR-L2 and CDR-L3 sequences set forth in SEQ ID NOS: 38, 39, and 40, respectively.

10. The bispecific CAR of embodiments 6-9, wherein the extracellular binding domain comprises in order from the amino- to carboxy-terminus:
(i) the V_{H} region of the BAFF-R-binding domain, the V_{L} region of the CD19-binding domain, the V_{H} region of the CD19-binding domain, and the V_{L} region of the BAFF-R binding domain;
(ii) the V_{L} region of the BAFF-R-binding domain, the V_{L} region of the CD19-binding domain, the V_{H} region of the CD19-binding domain, and the V_{H} region of the BAFF-R binding domain;
(iii) the V_{H} region of the BAFF-R-binding domain, the V_{H} region of the CD19-binding domain, the V_{L} region of the CD19-binding domain, and the V_{L} region of the BAFF-R-binding domain;
(iv) the V_{L} region of the BAFF-R-binding domain, the V_{H} region of the CD19-binding domain, the V_{L} region of the CD19-binding domain, and the V_{H} region of the BAFF-R binding domain;
(v) the V_{L} region of the CD19-binding domain, the V_{H} region of the BAFF-R-binding domain, the V_{L} region of the BAFF-R-binding domain, and the V_{H} region of the CD19-binding domain;
(vi) the V_{H} region of the CD19-binding domain, the V_{H} region of the BAFF-R-binding domain, the V_{L} region of the BAFF-R-binding domain, and the V_{L} region of the CD19-binding domain;
(vii) the V_{L} region of the CD19-binding domain, the V_{H} region of the CD19-binding domain, the V_{H} region of the BAFF-R-binding domain, and the V_{L} region of the BAFF-R-binding domain;
(viii) the V_{L} region of the CD19-binding domain, the V_{H} region of the CD19-binding domain, the V_{L} region of the BAFF-R-binding domain, and the V_{H} region of the BAFF-R-binding domain;
(ix) the V_{H} region of the CD19-binding domain, the V_{L} region of the CD19-binding domain, the V_{H} region of the BAFF-R-binding domain, and the V_{L} region of the BAFF-R-binding domain;
(x) the V_{H} region of the CD19-binding domain, the V_{L} region of the CD19-binding domain, the V_{L} region of the BAFF-R-binding domain, and the V_{H} region of the BAFF-R-binding domain;
(xi) the V_{L} region of the BAFF-R-binding domain, the V_{H} region of the BAFF-R-binding domain, the V_{H} region of the CD19-binding domain, and the V_{L} region of the CD19-binding domain;
(xii) the V_{L} region of the BAFF-R-binding domain, the V_{H} region of the BAFF-R-binding domain, the V_{L} region of the CD19-binding domain, and the V_{H} region of the CD19-binding domain;
(xiii) the V_{H} region of the BAFF-R-binding domain, the V_{L} region of the BAFF-R-binding domain, the V_{H} region of the CD19-binding domain, and the V_{L} region of the CD19-binding domain;
(xiv) the V_{H} region of the BAFF-R-binding domain, the V_{L} region of the BAFF-R-binding domain, the V_{L} region of the CD19-binding domain, and the V_{H} region of the CD19-binding domain;
(xv) the V_{L} region of the CD19-binding domain, the V_{L} region of the BAFF-R-binding domain, the V_{H} region of the BAFF-R-binding domain, and the V_{H} region of the CD19-binding domain; or
(xvi) the V_{H} region of the CD19-binding domain, the V_{L} region of the BAFF-R-binding domain, the V_{H} region of the BAFF-R-binding domain, and the V_{L} region of the CD19-binding domain.

11. The bispecific CAR of any of embodiments 1-10, wherein:
(i) the V_{H} region of the BAFF-R-binding domain comprises the sequence set forth in, or a sequence that is at least 85%, 86%, 87%, 88%, 89%, 90%, 91%, 92%, 93%, 94%, 95%, 96%, 97%, 98% or 99% identical to, SEQ ID NO:1, and the V_{L} region of the BAFF-R-binding domain comprises the sequence set forth in, or a sequence that is at least 85%, 86%, 87%, 88%, 89%, 90%, 91%, 92%, 93%, 94%, 95%, 96%, 97%, 98% or 99% identical to, SEQ ID NO:2;
(ii) the V_{H} region of the BAFF-R-binding domain comprises the sequence set forth in, or a sequence that is at least 85%, 86%, 87%, 88%, 89%, 90%, 91%, 92%, 93%, 94%, 95%, 96%, 97%, 98% or 99% identical to, SEQ ID NO:3, and the V_{L} region of the BAFF-R-binding domain comprises the sequence set forth in, or a sequence that is at least 85%, 86%, 87%, 88%, 89%, 90%, 91%, 92%, 93%, 94%, 95%, 96%, 97%, 98% or 99% identical to, SEQ ID NO:4;
(iii) the V_{H} region of the BAFF-R-binding domain comprises the sequence set forth in, or a sequence that is at least 85%, 86%, 87%, 88%, 89%, 90%, 91%, 92%, 93%, 94%, 95%, 96%, 97%, 98% or 99% identical to, SEQ ID NO:5, and the V_{L} region of the BAFF-R-binding domain comprises the sequence set forth in, or a sequence that is at least 85%, 86%, 87%, 88%, 89%, 90%, 91%, 92%, 93%, 94%, 95%, 96%, 97%, 98% or 99% identical to, SEQ ID NO:6;
(iv) the V_{H} region of the BAFF-R-binding domain comprises the sequence set forth in, or a sequence that is at least 85%, 86%, 87%, 88%, 89%, 90%, 91%, 92%, 93%, 94%, 95%, 96%, 97%, 98% or 99% identical to, SEQ ID NO:7, and the V_{L} region of the BAFF-R-binding domain comprises the sequence set forth in, or a sequence that is at least 85%, 86%, 87%, 88%, 89%, 90%, 91%, 92%, 93%, 94%, 95%, 96%, 97%, 98% or 99% identical to, SEQ ID NO:8; or
(v) the V_{H} region of the BAFF-R-binding domain comprises the sequence set forth in, or a sequence that is at least 85%, 86%, 87%, 88%, 89%, 90%, 91%, 92%, 93%, 94%, 95%, 96%, 97%, 98% or 99% identical to, SEQ ID NO:9, and the V_{L} region of the BAFF-R-binding domain comprises the sequence set forth in, or a sequence that is at least 85%, 86%, 87%, 88%, 89%, 90%, 91%, 92%, 93%, 94%, 95%, 96%, 97%, 98% or 99% identical to, SEQ ID NO:10.

12. The bispecific CAR of any of embodiments 1-11, wherein:
(i) the V_{H} region of the BAFF-R-binding domain comprises the sequence set forth in SEQ ID NO:1, and the V_{L} region of the BAFF-R-binding domain comprises the sequence set forth in SEQ ID NO:2;
(ii) the V_{H} of the BAFF-R-binding domain comprises the sequence set forth in SEQ ID NO: 3, and the V_{L} region of the BAFF-R-binding domain comprises the sequence set forth in SEQ ID NO:4;
(iii) the V_{H} region of the BAFF-R-binding domain comprises the sequence set forth in SEQ ID NO:5, and the V_{L} region of the BAFF-R-binding domain comprises the sequence set forth in SEQ ID NO:6;
(iv) the V_{H} region of the BAFF-R-binding domain comprises the sequence set forth in SEQ ID NO:7, and the V_{L} region of the BAFF-R-binding domain comprises the sequence set forth in SEQ ID NO:8; or
(v) the V_{H} region of the BAFF-R-binding domain comprises the sequence set forth in SEQ ID NO:9, and the V_{L} region of the BAFF-R-binding domain comprises the sequence set forth in SEQ ID NO:10.

13. The bispecific CAR of any of embodiments 1-12, wherein the extracellular binding domain comprises, from amino to carboxy terminus: the V_{H} region of the BAFF-R-binding domain, the V_{L} region of the CD19-binding domain, the V_{H} region of the CD19-binding domain, and the V_{L} region of the BAFF-R-binding domain.

14. The bispecific CAR of any of embodiments 1-12, wherein the extracellular binding domain comprises, from amino to carboxy terminus: the V_{L} region of the BAFF-R-binding domain, the V_{L} region of the CD19-binding domain, the V_{H} region of the CD19-binding domain, and the V_{H} region of the BAFF-R-binding domain.

15. The bispecific CAR of any of embodiments 6-12, wherein the extracellular binding domain comprises, from amino to carboxy terminus: the V_{L} region of the CD19-binding domain, the V_{H} region of the BAFF-R-binding domain, the V_{L} region of the BAFF-R-binding domain, and the V_{H} region of the CD19-binding domain.

16. The bispecific CAR of any of embodiments 6-12, wherein the extracellular binding domain comprises, from amino to carboxy terminus: the V_{H} region of the CD19-binding domain, the V_{H} region of the BAFF-R-binding domain, the V_{L} region of the BAFF-R-binding domain, and the V_{L} region of the CD19-binding domain.

17. The bispecific CAR of any of embodiments 6-12, wherein the extracellular binding domain comprises, from amino to carboxy terminus: the V_{L} region of the CD19-binding domain, the V_{H} region of the CD19-binding domain, the V_{H} region of the BAFF-R-binding domain, and the V_{L} region of the BAFF-R-binding domain.

18. The bispecific CAR of any of embodiments 6-12, wherein the extracellular binding domain comprises, from amino to carboxy terminus: the V_{L} region of the CD19-binding domain, the V_{H} region of the CD19-binding domain, the V_{L} region of the BAFF-R-binding domain, and the V_{H} region of the BAFF-R-binding domain.

19. The bispecific CAR of any of embodiments 1-18, wherein the V_{H} region of the BAFF-R-binding domain comprises CDR-H1, CDR-H2 and CDR-H3 sequences set forth in SEQ ID NOS: 22, 23, and 24, respectively; and the V_{L} region of the BAFF-R-binding domain comprises CDR-L1, CDR-L2 and CDR-L3 sequences set forth in SEQ ID NOS: 25, 26, and 27, respectively.

20. The bispecific CAR of any of embodiments 1-19, wherein the V_{H} region of the BAFF-R-binding domain comprises the sequence set forth in, or a sequence that is at least 85%, 86%, 87%, 88%, 89%, 90%, 91%, 92%, 93%, 94%, 95%, 96%, 97%, 98% or 99% identical to, SEQ ID NO:3, and the V_{L} region of the BAFF-R-binding domain comprises the sequence set forth in, or a sequence that is at least 85%, 86%, 87%, 88%, 89%, 90%, 91%, 92%, 93%, 94%, 95%, 96%, 97%, 98% or 99% identical to, SEQ ID NO:4.

21. The bispecific CAR of any of embodiments 1-20, wherein the V_{H} of the BAFF-R-binding domain comprises the sequence set forth in SEQ ID NO:3, and the V_{L} region of the BAFF-R-binding domain comprises the sequence set forth in SEQ ID NO:4.

22. The bispecific CAR of any of embodiments 1-18, wherein the V_{H} region of the BAFF-R-binding domain comprises the sequence set forth in, or a sequence that is at least 85%, 86%, 87%, 88%, 89%, 90%, 91%, 92%, 93%, 94%, 95%, 96%, 97%, 98% or 99% identical to, SEQ ID NO:5, and the V_{L} region of the BAFF-R-binding domain comprises the sequence set forth in, or a sequence that is at least 85%, 86%, 87%, 88%, 89%, 90%, 91%, 92%, 93%, 94%, 95%, 96%, 97%, 98% or 99% identical to, SEQ ID NO:6.

23. The bispecific CAR of any of embodiments 1-18 and 22, wherein the V_{H} region of the BAFF-R-binding domain comprises CDR-H1, CDR-H2 and CDR-H3 sequences set forth in SEQ ID NOS: 28, 29, and 30, respectively; and the V_{L} region of the BAFF-R-binding domain comprises CDR-L1, CDR-L2 and CDR-L3 sequences set forth in SEQ ID NOS: 31, 26, and 27, respectively.

24. The bispecific CAR of any of embodiments 1-18, 22, and 23, wherein the V_{H} region of the BAFF-R-binding domain comprises the sequence set forth in SEQ ID NO:5, and the V_{L} region of the BAFF-R-binding domain comprises the sequence set forth in SEQ ID NO: 6.

25. The bispecific CAR of any of embodiments 1-18, wherein the V_{H} region of the BAFF-R-binding domain comprises CDR-H1, CDR-H2 and CDR-H3 sequences set forth in SEQ ID NOS: 16, 17, and 18, respectively; and the V_{L} region of the BAFF-R-binding domain comprises CDR-L1, CDR-L2 and CDR-L3 sequences set forth in SEQ ID NOS: 19, 20, and 21, respectively.

26. The bispecific CAR of any of embodiments 1-18 and 25, wherein the V_{H} region of the BAFF-R-binding domain comprises the sequence set forth in, or a sequence that is at least 85%, 86%, 87%, 88%, 89%, 90%, 91%, 92%, 93%, 94%, 95%, 96%, 97%, 98% or 99% identical to, SEQ ID NO:1, and the V_{L} region of the BAFF-R-binding domain comprises the sequence set forth in, or a sequence that is at least 85%, 86%, 87%, 88%, 89%, 90%, 91%, 92%, 93%, 94%, 95%, 96%, 97%, 98% or 99% identical to, SEQ ID NO:2.

27. The bispecific CAR of any of embodiments 1-18, 25, and 26, wherein the V_{H} region of the BAFF-R-binding domain comprises the sequence set forth in SEQ ID NO:1, and the V_{L} region of the BAFF-R-binding domain comprises the sequence set forth in SEQ ID NO: 2.

28. The bispecific CAR of any of embodiments 1-18, wherein the V_{H} region of the BAFF-R-binding domain comprises CDR-H1, CDR-H2 and CDR-H3 sequences set forth in SEQ ID NOS: 22, 32 and 24, respectively; and the V_{L} region of the BAFF-R-binding domain comprises CDR-L1, CDR-L2 and CDR-L3 sequences set forth in SEQ ID NOS: 33, 26, and 34, respectively.

29. The bispecific CAR of any of embodiments 1-18 and 28, wherein the V_{H} region of the BAFF-R-binding domain comprises the sequence set forth in, or a sequence that is at least 85%, 86%, 87%, 88%, 89%, 90%, 91%, 92%, 93%, 94%, 95%, 96%, 97%, 98% or 99% identical to, SEQ ID NO:7, and the V_{L} region of the BAFF-R-binding domain comprises the sequence set forth in, or a sequence that is at least 85%, 86%, 87%, 88%, 89%, 90%, 91%, 92%, 93%, 94%, 95%, 96%, 97%, 98% or 99% identical to, SEQ ID NO:8.

30. The bispecific CAR of any of embodiments 1-18, 28, and 29, wherein the V_{H} region of the BAFF-R-binding domain comprises the sequence set forth in SEQ ID NO:7, and the V_{L} region of the BAFF-R-binding domain comprises the sequence set forth in SEQ ID NO: 8.

31. The bispecific CAR of any of embodiments 1-18, wherein the V_{H} region of the BAFF-R-binding domain comprises CDR-H1, CDR-H2 and CDR-H3 sequences set forth in SEQ ID NOS: 35, 36, and 37, respectively; and the V_{L} region of the BAFF-R-binding domain comprises CDR-L1, CDR-L2 and CDR-L3 sequences set forth in SEQ ID NOS: 38, 39, and 40, respectively.

32. The bispecific CAR of any of embodiments 1-18 and 31, wherein the V_{H} region of the BAFF-R-binding domain comprises the sequence set forth in, or a sequence that is at least 85%, 86%, 87%, 88%, 89%, 90%, 91%, 92%, 93%, 94%, 95%, 96%, 97%, 98% or 99% identical to, SEQ ID NO:9, and the V_{L} region of the BAFF-R-binding domain comprises the sequence set forth in, or a sequence that is at least 85%, 86%, 87%, 88%, 89%, 90%, 91%, 92%, 93%, 94%, 95%, 96%, 97%, 98% or 99% identical to, SEQ ID NO:10.

33. The bispecific CAR of any of embodiments 1-18, 31, and 32, wherein the V_{H} region of the BAFF-R-binding domain comprises the sequence set forth in SEQ ID NO:9, and the V_{L} region of the BAFF-R-binding domain comprises the sequence set forth in SEQ ID NO: 10.

34. The bispecific CAR of any of embodiments 1-33, wherein the V_{H} region of the BAFF-R binding domain is joined to the V_{L} region of the BAFF-R binding domain via an intradomain linker.

35. The bispecific CAR of any one of embodiments 1-34, wherein the V_{H} region of the CD19-binding domain comprises a CDR-H1, a CDR-H2 and a CDR-H3 each comprising a sequence that is contained within SEQ ID NO:41, and the V_{L} region of the CD19-binding domain comprises a CDR-L1, a CDR-L2 and a CDR-L3 each comprising a sequence that is contained within SEQ ID: NO 42.

36. The bispecific CAR of embodiment 35, wherein each CDR is defined in accordance with the Kabat definition, the Chothia definition, the combination of the Kabat and the Chothia definition, the AbM definition, or the contact definition.

37. The bispecific CAR of any of embodiments 1-36, wherein the V_{H} region of the CD19-binding domain comprises CDR-H1, CDR-H2 and CDR-H3 sequences set forth in SEQ ID NOS: 41, 44 and 46, respectively; and the V_{L} region of the CD19-binding domain comprises CDR-L1, CDR-L2 and CDR-L3 sequences set forth in SEQ ID NOS: 47, 49, and 51, respectively.

38. The bispecific CAR of any one of embodiments 1-37, wherein
(i) the V_{H} region of the CD19-binding domain comprises the sequences set forth in, or a sequence that is at least 90%, 91%, 92%, 93%, 94%, 95%, 96%, 97%, 98% or 99% identical to, SEQ ID NO: 41; and
(ii) the V_{L} region of the CD19-binding domain comprises the sequences set forth in, or a sequence that is at least 90%, 91%, 92%, 93%, 94%, 95%, 96%, 97%, 98% or 99% identical to, SEQ ID NO:42.

39. The bispecific CAR of any one of embodiments 1-38, wherein the V_{H} region of the CD19-binding domain comprises the sequence set forth in SEQ ID NO: 41; and the V_{L} region of the CD19-binding domain comprises the sequence set forth in SEQ ID NO:42.

40. A bispecific CAR comprising an extracellular binding domain, a spacer, a transmembrane domain, and an intracellular signaling domain, wherein the extracellular binding domain comprises:
a B-cell activating factor receptor (BAFF-R)-binding domain that binds to BAFF-R comprising a heavy chain variable (V_{H}) region and a light chain variable (V_{L}) region; and
a CD19-binding domain that binds to CD19 comprising a V_{H} region and a V_{L} region,
wherein the extracellular binding domain comprises in order from amino to carboxy terminus:
the V_{H} region of the BAFF-R-binding domain comprising the sequence set forth in SEQ ID NO: 3, the V_{L} region of the CD19-binding domain comprising the sequence set forth in SEQ ID NO: 42, the V_{H} region of the CD19-binding domain comprising the sequence set forth in SEQ ID NO: 41, and the V_{L} region of the BAFF-R-binding domain comprising the sequence set forth in SEQ ID NO: 4.

41. A bispecific CAR comprising an extracellular binding domain, a spacer, a transmembrane domain, and an intracellular signaling domain, wherein the extracellular binding domain comprises:
a BAFF-R-binding domain comprising V_{H} and V_{L}; and
a CD19-binding domain comprising V_{H} and V_{L},
wherein the extracellular binding domain comprises in order from amino to carboxy terminus:
the V_{L} region of the BAFF-R-binding domain comprising the sequence set forth in SEQ ID NO: 6, the V_{L} region of the CD19-binding domain comprising the sequence set forth in SEQ ID NO: 42, the V_{H} region of the CD19-binding domain comprising the sequence set forth in SEQ ID NO: 41, and the V_{H} region of the BAFF-R-binding domain comprising the sequence set forth in SEQ ID NO: 5.

42. The bispecific CAR of any of embodiments 1-41, wherein the V_{H} region of the CD19-binding domain is joined to the V_{L} region of the CD19-binding domain via an intradomain linker.

43. The bispecific CAR of embodiment 34 or embodiment 42, wherein the intradomain linker is a flexible linker.

44. The bispecific CAR of any of embodiments 34, 42, and 43, wherein the intradomain linker is 5 to 25 amino acids in length.

45. The bispecific CAR of any of embodiments 34 and 42-44, wherein the intradomain linker is 12 to 18 amino acids in length.

46. The bispecific CAR of any of embodiments 34, and 42-45, wherein the intradomain linker comprises the sequence set forth in SEQ ID NO:58.

47. The bispecific CAR of any of embodiments 34 and 42-45, wherein the intradomain linker comprises the sequence set forth in SEQ ID NO:59.

48. The bispecific CAR of any of embodiments 1-47, wherein the V_{H} region or the V_{L} region of the BAFF-R-binding domain are joined by an interdomain linker to the V_{H} region or the V_{L} region of the CD19-binding domain.

49. The bispecific CAR of any of embodiments 1-48, wherein:
(i) the V_{H} region of the BAFF-R binding domain is joined to the V_{L} region of the CD19-binding domain by an interdomain linker;
(i) the V_{H} region of the BAFF-R binding domain is joined to the V_{H} region of the CD19-binding domain by an interdomain linker;
(iii) the V_{L} region of the BAFF-R binding domain is joined to the V_{L} region of the CD19-binding domain by an interdomain linker; or
(iv) the V_{L} region of the BAFF-R binding domain is joined to the V_{H} region of the CD19-binding domain by an interdomain linker.

50. The bispecific CAR of embodiment 48 or embodiment 49, wherein the interdomain linker is a flexible peptide linker.

51. The bispecific CAR of any of embodiments 48-50, wherein the length of the interdomain linker is between 5 and 25 amino acids, inclusive.

52. The bispecific CAR of any of embodiments 48-51, wherein the length of the interdomain linker is between 5 and 15 amino acids, inclusive.

53. The bispecific CAR of any of embodiments 34 and 48-52, wherein the interdomain linker is a G4S linker (SEQ ID NO:60), a G4S2 linker (SEQ ID NO:61) or a (G4S)4 linker (SEQ ID NO:62).

54. The bispecific CAR of any one of embodiments 48-53, wherein the interdomain linker is set forth in SEQ ID NO: 60.

55. The bispecific CAR of any one of embodiments 48-54, wherein the interdomain linker is set forth in SEQ ID NO: 61.

56. The bispecific CAR of any of embodiments 1-55, wherein the spacer is interposed between the extracellular binding domain and the transmembrane domain.

57. The bispecific CAR of any one of embodiments 1-56, wherein the spacer comprises a hinge region sequence.

58. The bispecific CAR of any one of embodiments 1-57, wherein the spacer comprises a hinge region of an immunoglobulin or a variant thereof.

59. The bispecific CAR of embodiment 52, wherein the hinge region of an immunoglobulin is an IgG4 hinge region, optionally a human IgG4 hinge region, or a variant thereof.

60. The bispecific CAR of any one of embodiments 1-59, wherein the spacer comprises a variant IgG4 hinge region comprising substitution of amino acids CPSC to CPPC compared to the wild-type IgG4 hinge region.

61. The bispecific CAR of any one of embodiments 1-60, wherein the spacer is less than at or about 15 amino acids in length.

62. The bispecific CAR of any one of embodiments 1-61, wherein the spacer is between 12 and 15 amino acids in length.

63. The bispecific CAR of any one of embodiments 1-62, wherein the spacer comprises an amino acid sequence having at least at or about 85%, 86%, 87%, 88%, 89%, 90%, 91%, 92%, 93%, 94%, 95%, 96%, 97%, 98% or 99% identity to SEQ ID NO: 52, optionally wherein the spacer has the sequence set forth in SEQ ID NO:52.

64. The bispecific CAR of any one of embodiments 1-60, wherein the spacer is between 200 and 250 amino acids in length, or between 220 and 240 amino acids in length.

65. The bispecific CAR of any one of embodiments 1-60 and 64 wherein the spacer comprises a hinge region of an immunoglobulin, a CH2 region of an immunoglobulin or a chimeric CH2 region of two different immunoglobulins, and a CH3 region of an immunoglobulin.

66. The bispecific CAR of embodiment 65, wherein the spacer comprises an IgG4 hinge region or a variant thereof, a chimeric CH2 region comprising a portion of an IgG4 CH2 and a portion of an IgG2 CH2 (IgG2/4 CH2 region), and an IgG4 CH3 region.

67. The bispecific CAR of any one of embodiments 1-62 and 64-66, wherein the spacer comprises an amino acid sequence having at least at or about 85%, 86%, 87%, 88%, 89%, 90%, 91%, 92%, 93%, 94%, 95%, 96%, 97%, 98% or 99% identity to SEQ ID NO:53, optionally wherein the spacer has the sequence set forth in SEQ ID NO: 53.

68. The bispecific CAR of any one of embodiments 1-67 wherein the transmembrane domain comprises a transmembrane domain from CD28, optionally a human CD28.

69. The bispecific CAR of any one of embodiments 1-68, wherein the transmembrane domain is or comprises SEQ ID NO: 55 or an amino acid sequence having at least at or about 90%, 91%, 92%, 93%, 94%, 95%, 96%, 97%, 98% or 99% sequence identity to SEQ ID NO: 55.

70. The bispecific CAR of any one of embodiments 1-69, wherein the transmembrane domain is set forth in SEQ ID NO: 55.

71. The bispecific CAR of any one of embodiments 1-70, wherein the intracellular signaling domain comprises an intracellular signaling domain capable of inducing a primary activation signal in a T cell.

72. The bispecific CAR of embodiment 71, wherein the intracellular signaling domain is a domain from a T cell receptor (TCR) component and/or comprises an immunoreceptor tyrosine-based activation motif (ITAM).

73. The bispecific CAR of embodiment 71 or embodiment 72, wherein the intracellular signaling domain is a cytoplasmic signaling domain of a CD3-zeta (CD3ζ) chain, optionally a human CD3ζ chain.

74. The bispecific CAR of any one of embodiments 71-73, wherein the intracellular signaling domain comprises the sequence set forth in SEQ ID NO: 57, or an amino acid sequence having at least at or about 90%, 91%, 92%, 93%, 94%, 95%, 96%, 97%, 98% or 99% sequence identity to SEQ ID NO: 57.

75. The bispecific CAR of any one of embodiments 71-74, wherein the intracellular signaling domain is set forth in SEQ ID NO: 57.

76. The bispecific CAR of any one of embodiments 71-75, wherein the intracellular signaling region further comprises a costimulatory signaling region.

77. The bispecific CAR of embodiment 76, wherein the costimulatory signaling region is between the transmembrane region and the intracellular signaling domain.

78. The bispecific CAR of embodiment 76 or embodiment 77, wherein the costimulatory signaling region comprises an intracellular signaling domain of a T cell costimulatory molecule or a signaling portion thereof.

79. The bispecific CAR of any one of embodiments 76-78, wherein the costimulatory signaling region comprises an intracellular signaling domain of 4-1BB, optionally a human 4-1BB.

80. The bispecific CAR of any of embodiments 76-79, wherein the costimulatory signaling region comprises the sequence set forth in SEQ ID NO: 56 or an amino acid sequence having at least at or about 90%, 91%, 92%, 93%, 94%, 95%, 96%, 97%, 98% or 99% sequence identity to SEQ ID NO: 56.

81. The bispecific CAR of any of embodiments 76-80, wherein the costimulatory signaling region is set forth in SEQ ID NO: 56.

82. A bispecific CAR comprising the amino acid sequence set forth in SEQ ID NO: 94, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 94.

83. A bispecific CAR comprising the amino acid sequence set forth in SEQ ID NO: 95, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 95.

84. A bispecific CAR comprising the amino acid sequence set forth in SEQ ID NO: 96, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 96.

85. A bispecific CAR comprising the amino acid sequence set forth in SEQ ID NO: 97, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 97.

86. A bispecific CAR comprising the amino acid sequence set forth in SEQ ID NO: 98, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 98.

87. A bispecific CAR comprising the amino acid sequence set forth in SEQ ID NO: 99, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 99.

88. A bispecific CAR comprising the amino acid sequence set forth in SEQ ID NO: 100, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 100.

89. A bispecific CAR comprising the amino acid sequence set forth in SEQ ID NO: 101, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 101.

90. A bispecific CAR comprising the amino acid sequence set forth in SEQ ID NO: 102, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 102.

91. A bispecific CAR comprising the amino acid sequence set forth in SEQ ID NO: 103, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 103.

92. The bispecific CAR of any of embodiments 1-91, wherein the binding of the BAFF-R domain to BAFF in the presence of soluble BAFF is reduced no more than 10% relative to binding in the absence of soluble BAFF.

93. The bispecific CAR of any of embodiments 1, 3-13, 19-21, 35-40, 42-45, 47-54, and 56-92 wherein the binding of the CD19 domain to CD19 in the presence of FMC63 scFv is reduced no more than 30% relative to binding in the absence of FMC63 scFv. 94. A polynucleotide encoding the bispecific CAR of any one of embodiments 1-93.

95. The polynucleotide of embodiment 94, wherein the polynucleotide is optimized by splice site elimination.

96. The polynucleotide of embodiment 94 or embodiment 95, wherein the polynucleotide is codon-optimized for expression in a human cell.

97. A vector comprising the polynucleotide of any one of the embodiments 94-96.

98. The vector of embodiment 97, wherein the vector is a viral vector.

99. The vector of embodiment 98, wherein the viral vector is a retroviral vector (e.g., lentiviral vector).

100. A cell comprising the bispecific CAR of any one of embodiments 1-93.

101. A cell comprising the polynucleotide of any of embodiments 94-96 or the vector of any of embodiments 97-99.

102. The cell of embodiment 100 or 101, wherein the cell is an immune cell.

103. The cell of any one of embodiments 100-102, wherein the cell is a lymphocyte. 104. The cell of any one of embodiments 100-103, that is an NK cell or a T cell.

105. The cell of any one of embodiments 100-104, wherein the cell is a T cell.

106. The cell of any one of embodiments 100-105, wherein the T cell is a CD4+ T cell or a CD8+ T cell.

107. The cell of any one of embodiments 100-106, wherein the cell is a primary cell.

108. The cell of any one of embodiments 100-107, wherein the cell exhibits cytotoxic activity against CD19+ cells, BAFF-R+ cells, and CD19+/BAFF-R+ cells.

109. A composition comprising a plurality of cells of any one of embodiments 100-108.

110. The composition of embodiment 109, further comprising a pharmaceutically acceptable excipient.

111. The composition of embodiment 109 or 110, wherein the composition comprises CD4+ and CD8+ T cells.

112. The composition of embodiment 111, wherein the ratio of CD4+ to CD8+ T cells is from at or about 1:3 to 3:1, optionally at or about 1:2 to 2:1, optionally at or about 1:1.

113. The composition of any one of embodiments 109-112, wherein greater than at or about 90%, greater than at or about 95% or greater than at or about 98% of cells in the composition are CD3+ T cells.

114. The composition of any one of embodiments 109-113, wherein at least 40%, at least 50%, at least 60%, at least 70%, at least 80% or at least 90% of cells in the composition express the CAR.

115. The composition of any one of embodiments 109-114, wherein, among a plurality of the cells in the composition expressing the bispecific CAR, less than at or about 10%, at or about 9%, at or about 8%, at or about 7%, at or about 5%, at or about 4%, at or about 3%, at or about 2% or at or about 1% of the cells in the plurality exhibits tonic signaling and/or antigen independent activity or signaling.

116. The composition of any one of embodiments 109-115, wherein the composition comprises between about 1.0×10⁷ bispecific CAR-expressing T cells and 1.2×10⁹ bispecific CAR-expressing T cells, between about 1.0×10⁷ bispecific CAR-expressing T cells and 6.5×10⁸ bispecific CAR-expressing T cells, between about 1.5×10⁷ bispecific CAR-expressing T cells and 6.5×10⁸ bispecific CAR-expressing T cells, between about 1.5×10⁷ bispecific CAR-expressing T cells and 6.0×10⁸ bispecific CAR-expressing T cells, between about 2.5×10⁷ bispecific CAR-expressing T cells and 6.0×10⁸ bispecific CAR-expressing T cells, between about 5.0×10⁷ bispecific CAR-expressing T cells and 6.0×10⁸ bispecific CAR-expressing T cells, between about 1.25×10⁷ bispecific CAR-expressing T cells and 1.2×10⁹ bispecific CAR-expressing T cells, between about 1.5×10⁷ bispecific CAR-expressing T cells and 1.2×10⁹ bispecific CAR-expressing T cells, between about 5.0×10⁷ bispecific CAR-expressing T cells and 4.5×10⁸ bispecific CAR-expressing T cells, or between about 1.5×10⁸ bispecific CAR-expressing T cells and 3.0×10⁸ bispecific CAR-expressing T cells, each inclusive.

117. The composition of any one of embodiments 109-116, wherein the composition comprises at or about 1.5×10⁷, at or about 2.5×10⁷, at or about 5.0×10⁷, at or about 7.5×10⁷, at or about 1.5×10⁸, at or about 2.25×10⁸, at or about 3.0×10⁸, at or about 4.5×10⁸, at or about 6.0×10⁸, at or about 8.0×10⁸, or at or about 1.2×10⁹ bispecific CAR-expressing T cells.

118. A method of treating a disease or disorder in a subject, the method comprising administering the cell of any of embodiments 100-108 or the composition of any of embodiments 107-115 to a subject in need of treatment thereof.

119. A method of treatment, comprising administering the bispecific CAR of any of embodiments 1-93, the polynucleotide of any one of embodiments 94-96, or the vector of any one of embodiments 97-99 to a subject having a disease or disorder.

120. The method of embodiment 118 or embodiment 119, wherein the disease or disorder is a cancer or an autoimmune disease.

121. The method of embodiment 120, wherein the disease or disorder is a cancer.

122. The method of embodiment 120 or 121, wherein the cancer is a BAFF-R-expressing cancer, a CD19-expressing cancer, or a BAFF-R- and CD19-expressing cancer.

123. The method of any of embodiments 120-122, wherein the cancer is a lymphoma or a leukemia.

124. The method of embodiment 123, wherein the lymphoma is a large B cell lymphoma.

125. The method of embodiment 123, wherein the lymphoma is a non-Hodgkin lymphoma.

126. The method of embodiment 120, wherein the disease or disorder is a an autoimmune disease.

127. The cell of any of embodiments 100-108 or the composition of any of embodiments 109-117 for use in treating a disease or disorder.

128. Use of the cell of any of embodiments 100-108 or the composition of any of embodiments 109-117 for the manufacture of a medicament for treating a disease or disorder.

129. Use of the cell of any of embodiments 100-108 or the composition of any of embodiments 109-117 for the treatment of a disease or disorder.

130. The bispecific CAR of any one of embodiments 1-93, the polynucleotide of any one of embodiments 94-96, or the vector of any one of embodiments 97-99 for use in treating a disease or disorder.

131. Use of the bispecific CAR of any of embodiments 1-93, the polynucleotide of any one of embodiments 94-96, or the vector of any one of embodiments 97-99 for the manufacture of a medicament for treating a disease or disorder.

132. Use of the bispecific CAR of any of embodiments 1-93, the polynucleotide of any one of embodiments 94-96, or the vector of any one of embodiments 97-99 for the treatment of a disease or disorder.

133. The use of embodiment 126 or embodiment 127, wherein the disease or disorder is a cancer or an autoimmune disease.

134. The use of embodiment 133, wherein the disease or disorder is a cancer.

135. The use of embodiment 133 or 134, wherein the cancer is a BAFF-R-expressing cancer, a CD19-expressing cancer, or a BAFF-R- and CD19-expressing cancer.

136. The use of any of embodiments 133-135, wherein the cancer is a lymphoma or a leukemia.

137. The use of embodiment 136, wherein the lymphoma is a large B cell lymphoma.

138. The use of embodiment 136, wherein the lymphoma is a non-Hodgkin lymphoma.

139. The use of embodiment 133, wherein the disease or disorder is a an autoimmune disease.

140. The use of embodiment 128 or 129, wherein the disease or disorder is a cancer or an autoimmune disease.

141. The use of embodiment 140, wherein the disease or disorder is a cancer.

142. The use of embodiment 140 or 141, wherein the cancer is a BAFF-R-expressing cancer, a CD19-expressing cancer, or a BAFF-R- and CD19-expressing cancer.

143. The use of any of embodiments 140-142, wherein the cancer is a lymphoma or a leukemia.

144. The use of embodiment 143, wherein the lymphoma is a large B cell lymphoma.

145. The use of embodiment 143, wherein the lymphoma is a non-Hodgkin lymphoma.

146. The use of embodiment 140, wherein the disease or disorder is an autoimmune disease.

147. A kit comprising the bispecific CAR of any one of embodiments 1-93, the polynucleotide of any one of embodiments 94-96, the vector of any one of embodiments 97-99, the cell of any one of embodiments 100-108, or the composition of any one of embodiments 109-117, and instructions for use, optionally wherein the instructions are for administering the bispecific CAR, the cell, or the composition, optionally in accord with the method, the cell, the composition, the bispecific CAR, the polynucleotide, or the vector for use or the use of any of embodiments 128-146.

148. An article of manufacture comprising the bispecific CAR of any one of embodiments 1-93, the polynucleotide of any one of embodiments 94-96, the vector of any one of embodiments 97-99, the cell of any one of embodiments 100-108, or the composition of any one of embodiments 109-117 or the kit of embodiment 147.

149. An antibody or antigen-binding portion thereof that binds B-cell activating factor receptor (BAFF-R), comprising a heavy chain variable region (V_{H}) and a light chain variable region (V_{L}), wherein
(i) V_{H} comprises CDR-H1, CDR-H2, CDR-H3 each having a sequence that is contained within SEQ ID NO:1, and the V_{L} region comprises a light chain complementarity determining region 1 (CDR-L1), a light chain complementarity determining region 2 (CDR-L2) and a light chain complementarity determining region 3 (CDR-L3) contained within SEQ ID NO: 2;
(ii) the V_{H} region comprises a CDR-H1, a CDR-H2 and a CDR-H3 contained within SEQ ID NO:3, and the V_{L} region comprises a CDR-L1, a CDR-L2 and a CDR-L3 contained within SEQ ID: NO 4;
(iii) the V_{H} region comprises a CDR-H1, a CDR-H2 and a CDR-H3 contained within SEQ ID NO:5, and the V_{L} region comprises a CDR-L1, a CDR-L2 and a CDR-L3 contained within SEQ ID: NO 6;
(iv) the V_{H} region comprises a CDR-H1, a CDR-H2 and a CDR-H3 contained within SEQ ID NO:7, and the V_{L} region comprises a CDR-L1, a CDR-L2 and a CDR-L3 contained
(v) V_{H} comprises a CDR-H1, a CDR-H2 and a CDR-H3 contained within SEQ ID NO: 9, and the V_{L} region comprises a CDR-L1, a CDR-L2 and a CDR-L3 contained within SEQ ID: NO 10.

150. The antibody or antigen-binding portion thereof of embodiment 149, wherein V_{H} comprises a CDR-H1, a CDR-H2, and a CDR-H3 each having a sequence that is contained within SEQ ID NO: 1, and V_{L} comprises a CDR-L1, a CDR-L2, and a CDR-L3 each having a sequence that is contained within SEQ ID: NO 2.

151. The antibody or antigen-binding portion thereof of embodiment 150, wherein V_{H} comprises a CDR-H1, a CDR-H2 and a CDR-H3 contained within SEQ ID NO: 3, and V_{L} comprises a CDR-L1, a CDR-L2 and a CDR-L3 contained within SEQ ID: NO 4.

152. The antibody or antigen-binding fragment thereof of embodiment 150, wherein the V_{H} region comprises a CDR-H1, a CDR-H2 and a CDR-H3 contained within SEQ ID NO: 5, and the V_{L} region comprises a CDR-L1, a CDR-L2 and a CDR-L3 contained within SEQ ID: NO 6.

153. The antibody or antigen-binding fragment thereof of embodiment 150, wherein the V_{H} region comprises a CDR-H1, a CDR-H2 and a CDR-H3 contained within SEQ ID NO: 7, and the V_{L} region comprises a CDR-L1, a CDR-L2 and a CDR-L3 contained within SEQ ID: NO 8.

154. The antibody or antigen-binding fragment thereof of embodiment 150, wherein the V_{H} region comprises a CDR-H1, a CDR-H2 and a CDR-H3 contained within SEQ ID NO: 9, and the V_{L} region comprises a CDR-L1, a CDR-L2 and a CDR-L3 contained within SEQ ID: NO 10.

155. The antibody or antigen-binding portion of any one of embodiment 150-154, wherein each CDR is defined in accordance with the Kabat definition, the Chothia definition, the combination of the Kabat and the Chothia definition, the AbM definition, or the contact definition.

156. The antibody or antigen-binding portion of any one of embodiments 149-154, wherein the antibody or antigen-binding portion thereof binds to BAFF-R with a K_{D} of about 10⁻⁷ M to about 10⁻¹¹ M.

157. An antibody or antigen-binding portion thereof that specifically binds BAFF-R, comprising V_{H} and V_{L}, wherein:
(i) V_{H} comprises a CDR-H1, a CDR-H2, and a CDR-H3 comprising the sequences set forth in SEQ ID NOS: 16, 17, and 18, respectively, and V_{L} comprises a CDR-L1, CDR-L2, and a CDR-L3 comprising the sequences set forth in SEQ ID NOS: 19, 20, and 21, respectively;
(ii) V_{H} comprises a CDR-H1, a CDR-H2, and a CDR-H3 comprising the sequences set forth in SEQ ID NOS: 22, 23, and 24, respectively, and V_{L} comprises a CDR-L1, a CDR-L2 and a CDR-L3 comprising the sequences set forth in SEQ ID NOS: 25, 26, and 27, respectively;
(iii) V_{H} comprises a CDR-H1, a CDR-H2, and a CDR-H3 comprising the sequences set forth in SEQ ID NOS: 28, 29, and 30, respectively, and V_{L} region comprises a CDR-L1, a CDR-L2 and a CDR-L3 comprising the sequences set forth in SEQ ID NOS: 31, 26, and 27, respectively;
(iv) V_{H} comprises a CDR-H1, a CDR-H2, and a CDR-H3 comprising the sequences set forth in SEQ ID NOS: 22, 32 and 24, respectively, and V_{L} comprises a CDR-L1, a CDR-L2 and a CDR-L3 comprising the sequences set forth in SEQ ID NOS: 33, 26, and 34, respectively; or
(v) V_{H} comprises a CDR-H1, a CDR-H2, and a CDR-H3 comprising the sequences set forth in SEQ ID NOS: 35, 36, and 37, respectively, and V_{L} comprises a CDR-L1, a CDR-L2, and a CDR-L3 comprising the sequences set forth in SEQ ID NOS: 38, 39, and 40, respectively.

158. The antibody or antigen-binding fragment thereof of any of embodiments 149, 150 and 157, wherein the V_{H} region comprises a CDR-H1, a CDR-H2, and a CDR-H3 comprising the sequence set forth in SEQ ID NOS: 16, 17, and 18, respectively, and the V_{L} region comprises a CDR-L1, a CDR-L2, and a CDR-L3 comprising the sequence set forth in SEQ ID NOS: 19, 20, and 21, respectively.

159. The antibody or antigen-binding fragment thereof of any of embodiments 149, 151 and 157, wherein the V_{H} region comprises a CDR-H1, a CDR-H2, and a CDR-H3 comprising the sequence set forth in SEQ ID NOS: 22, 23, and 24, respectively, and the V_{L} region comprises a CDR-L1, a CDR-L2, and a CDR-L3 comprising the sequence set forth in SEQ ID NOS: 25, 26, and 27, respectively.

160. The antibody or antigen-binding fragment thereof of any of embodiments 149, 152 and 157, wherein the V_{H} region comprises a CDR-H1, a CDR-H2, and a CDR-H3 comprising the sequence set forth in SEQ ID NOS: 28, 29, and 30, respectively, and the V_{L} region comprises a CDR-L1, a CDR-L2, and a CDR-L3 comprising the sequence set forth in SEQ ID NOS: 31, 26, and 27, respectively.

161. The antibody or antigen-binding fragment thereof of any of embodiments 149, 153 and 157, wherein the V_{H} region comprises a CDR-H1, a CDR-H2, and a CDR-H3 comprising the sequence set forth in SEQ ID NOS: 22, 32, and 24, respectively, and the V_{L} region comprises a CDR-L1, a CDR-L2, and a CDR-L3 comprising the sequence set forth in SEQ ID NOS: 33, 26, and 34, respectively.

162. The antibody or antigen-binding fragment thereof of any of embodiments 149, 154 and 157, wherein the V_{H} region comprises a CDR-H1, a CDR-H2, and a CDR-H3 comprising the sequence set forth in SEQ ID NOS: 35, 36, and 37, respectively, and the V_{L} region comprises a CDR-L1, a CDR-L2, and a CDR-L3 comprising the sequence set forth in SEQ ID NOS: 38, 39, and 40, respectively.

163. The antibody or antigen-binding portion thereof of embodiment 149 or embodiment 157 wherein:
(i) V_{H} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 1, and V_{L} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 2;
(ii) V_{H} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 3, and V_{L} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 4;
(iii) V_{H} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 5, and V_{L} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 6;
(iv) V_{H} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 7, and V_{L} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 8; or
(v) V_{H} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 9, and V_{L} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 10.

164. An antibody or antigen-binding portion thereof that specifically binds BAFF-R, comprising V_{H} and V_{L}, wherein:
(i) V_{H} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 1, and V_{L} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 2;
(ii) V_{H} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 3, and V_{L} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 4;
(iii) V_{H} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 5, and V_{L} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 6;
(iv) V_{H} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 7, and V_{L} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 8; or
(v) V_{H} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 9, and V_{L} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 10.

165. The antibody or antigen-binding portion thereof of any of embodiments 149, 150, 157, 158, 160, and 164, wherein V_{H} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 1, and V_{L} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 2.

166. The antibody or antigen-binding portion thereof of any of embodiments 149, 151, 157, 159, 163, and 164, wherein V_{H} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 3, and V_{L} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 4.

167. The antibody or antigen-binding portion thereof of any of embodiments 149, 152, 157, 160, 163, and 164, wherein V_{H} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 5, and V_{L} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 6.

168. The antibody or antigen-binding portion thereof of any of embodiments 149, 153, 157, 161, 163, and 164, wherein V_{H} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 7, and V_{L} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 8.

169. The antibody or antigen-binding portion thereof of any of embodiments 149, 154, 157, 162, 163, and 164, wherein V_{H} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 9, and V_{L} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 10.

170. The antibody or antigen-binding portion thereof of any of embodiments 149, 157, 163, and 164, wherein:
(i) V_{H} and V_{L} are or comprise the sequence set forth in SEQ ID NOS: 1 and 2, respectively;
(ii) V_{H} and V_{L} are or comprise the sequence set forth in SEQ ID NOS: 3 and 4, respectively;
(iii) V_{H} and V_{L} are or comprise the sequence set forth in SEQ ID NOS: 5 and 6, respectively;
(iv) V_{H} and V_{L} are or comprise the sequence set forth in SEQ ID NOS: 7 and 8, respectively; or
(v) V_{H} and V_{L} are or comprise the sequence set forth in SEQ ID NOS: 9 and 10, respectively.

171. An antibody or antigen-binding portion thereof that specifically binds BAFF-R, comprising V_{H} and V_{L}, wherein:
(i) V_{H} and V_{L} are or comprise the sequence set forth in SEQ ID NOS: 1 and 2, respectively;
(ii) V_{H} and V_{L} are or comprise the sequence set forth in SEQ ID NOS: 3 and 4, respectively;
(iii) V_{H} and V_{L} are or comprise the sequence set forth in SEQ ID NOS: 5 and 6, respectively;
(iv) V_{H} and V_{L} are or comprise the sequence set forth in SEQ ID NOS: 7 and 8, respectively; or
(v) V_{H} and V_{L} are or comprise the sequence set forth in SEQ ID NOS: 9 and 10, respectively.

172. The antibody or antigen-binding portion thereof of any of embodiments 149, 150, 157, 158, 163, 164, 165, 170, and 171, wherein V_{H} and V_{L} are or comprise the sequence set forth in SEQ ID NOS: 1 and 2, respectively.

173. The antibody or antigen-binding portion thereof of any of embodiments 149, 151, 157, 159, 163, 164, 166, 170, and 171, wherein V_{H} and V_{L} are or comprise the sequence set forth in SEQ ID NOS: 3 and 4, respectively.

174. The antibody or antigen-binding portion thereof of any of embodiments 149, 152, 157, 160, 163, 164, 167, 170, and 171, wherein V_{H} and V_{L} are or comprise the sequence set forth in SEQ ID NOS: 5 and 6, respectively.

175. The antibody or antigen-binding portion thereof of any of embodiments 149, 153, 157, 161, 163, 164, 168, 170, and 171, wherein V_{H} and V_{L} are or comprise the sequence set forth in SEQ ID NOS: 7 and 8, respectively.

176. The antibody or antigen-binding portion thereof of any of embodiments 149, 154, 157, 162, 163, 164, 169, 170, and 171, wherein V_{H} and V_{L} are or comprise the sequence set forth in SEQ ID NOS: 9 and 10, respectively.

177. The antibody or antigen-binding portion thereof of any of embodiments 149-176, wherein the antibody is a full-length antibody.

178. The antibody or antigen-binding portion thereof of any of embodiments 149-176, wherein the antibody is an antigen-binding fragment.

179. The antibody or antigen-binding portion thereof of any of embodiments 149-178, wherein said anti-BAFF-R antibody or antigen-binding portion thereof is recombinant.

180. The antibody or antigen-binding portion thereof of any of embodiments 149-179, wherein V_{H} and V_{L} are human or are derived from a human protein.

181. The antibody or antigen-binding portion thereof of any of embodiments 149-176 and 177-180, wherein the antigen-binding portion thereof comprises a single chain variable fragment (scFv).

182. The antibody or antigen-binding portion thereof of embodiment 181, wherein V_{H} is amino-terminal to V_{L}.

183. The antibody or antigen-binding portion thereof of embodiment 181, wherein V_{H} is carboxy-terminal to V_{L}.

184. The antibody or antigen-binding portion thereof of any of embodiments 179-181, when V_{H} and V_{L} are joined by a flexible linker.

185. The antibody or antigen-binding portion thereof of embodiment 184, wherein the flexible linker comprises the sequence set forth in SEQ ID NO: 58.

186. The antibody or antigen-binding portion thereof of any of embodiments 179-185, wherein the antigen-binding portion is or comprises the sequence set forth in SEQ ID NO: 11, 12, 13, 14, or 15, or an amino acid sequence having at least at or about 85%, 86%, 87%, 88%, 89%, 90%, 91%, 92%, 93%, 94%, 95%, 96%, 97%, 98% or 99% sequence identity to SEQ ID NO: 11, 12, 13, 14, or 15.

187. The antibody or antigen-binding portion thereof of any of embodiments 179-185, wherein the antigen-binding portion is or comprises the sequence set forth in SEQ ID NO: 11.

188. The antibody or antigen-binding portion thereof of any of embodiments 179-185, wherein the antigen-binding portion is or comprises the sequence set forth in SEQ ID NO: 12.

189. The antibody or antigen-binding portion thereof of any of embodiments 179-185, wherein the antigen-binding portion is or comprises the sequence set forth in SEQ ID NO: 13.

190. The antibody or antigen-binding portion thereof of any of embodiments 179-185, wherein the antigen-binding portion is or comprises the sequence set forth in SEQ ID NO: 14.

191. The antibody or antigen-binding portion thereof of any of embodiments 179-185, wherein the antigen-binding portion is or comprises the sequence set forth in SEQ ID NO: 15.

192. The antibody or antigen-binding portion thereof of any of embodiments 149-191, wherein said anti-BAFF-R antibody or antigen-binding portion thereof specifically binds to a human B-cell activating factor receptor (BAFF-R) protein.

193. The antibody or antigen-binding portion thereof of embodiment 192, wherein the human BAFF-R protein comprises an amino acid sequence set forth in SEQ ID NO: 120.

194. A pharmaceutical composition comprising the antibody or antigen-binding portion of any of embodiments 149-193, and a pharmaceutical carrier.

195. A chimeric antigen receptor (CAR) comprising an extracellular binding domain comprising an antibody or antigen-binding portion thereof of any of embodiments 149-193, a transmembrane domain, and an intracellular signaling domain.

196. The CAR of embodiment 195, wherein the intracellular signaling domain comprises an intracellular signaling domain capable of inducing a primary activation signal in a T cell.

197. The CAR of embodiment 195 or 196, wherein the intracellular signaling domain is a domain from a T cell receptor (TCR) component and/or comprises an immunoreceptor tyrosine-based activation motif (ITAM).

198. The CAR of any one of embodiments 195-197, wherein the intracellular signaling domain is a cytoplasmic signaling domain of a CD3-zeta (CD3ζ) chain, optionally a human CD3ζ chain.

199. The CAR of any one of embodiments 195-198, wherein the intracellular signaling region further comprises a costimulatory signaling region.

200. The CAR of embodiment 199, wherein the costimulatory signaling region is between the transmembrane region and the intracellular signaling domain.

201. The CAR of embodiment 199 or embodiment 200, wherein the costimulatory signaling region comprises an intracellular signaling domain of a T cell costimulatory molecule or a signaling portion thereof.

202. The CAR of any one of embodiments 199-201, wherein the costimulatory signaling region comprises an intracellular signaling domain of 4-1BB, optionally a human 4-1BB.

203. A conjugate, comprising the antibody or antigen-binding portion thereof of any of embodiments 149-193 and a heterologous molecule or moiety.

204. The conjugate of embodiment 203, wherein the heterologous molecule or moiety is a therapeutic moiety.

205. A nucleic acid encoding the antibody or antigen-binding portion of any of embodiments 149-193.

206. A polynucleotide comprising a nucleic acid of embodiment 205.

207. A polynucleotide comprising a nucleic acid encoding the conjugate of embodiment 203 or embodiment 204.

208. The polynucleotide of embodiment 206 or embodiment 207, wherein the polynucleotide is optimized by splice site elimination.

209. The polynucleotide of any one of embodiments 206-208, wherein the polynucleotide is codon-optimized for expression in a human cell.

210. An expression vector comprising the nucleic acid of embodiment 205.

211. A vector, comprising the polynucleotide of any of embodiments 206-209.

212. The vector of embodiment 211, wherein the vector is a viral vector.

213. The vector of embodiment 212, wherein the viral vector is a retroviral vector or a lentiviral vector.

214. A cell comprising the antibody or antigen-binding portion thereof of any of embodiments 149-193, the CAR of any one of embodiments 195-202, or the conjugate of embodiment 203 or embodiment 204.

215. A cell comprising the polynucleotide of any of embodiments 206-209, or the vector of any of embodiments 210-213.

216. The cell of embodiment 215, wherein the cell is an immune cell.

217. The cell of embodiment 215 or embodiment 216, that is a lymphocyte.

218. The cell of any one of embodiments 215-217, that is an NK cell or a T cell.

219. The cell of any one of embodiments 215-218, wherein the cell is a T cell and the T cell is a CD4+ T cell or a CD8+ T cell.

220. The cell of any of embodiments 214-219, wherein the cell is a primary cell obtained from a subject.

221. A composition comprising the cell of any of embodiments 214-220.

222. A composition comprising the antibody or antigen-binding portion thereof of any of embodiments 149-193, the CAR of any one of embodiments 195-202, or the conjugate of embodiment 203 or embodiment 204.

223. The composition of embodiment 212 or embodiment 213, further comprising a pharmaceutically acceptable excipient.

224. The composition of embodiment 221 or embodiment 222, wherein the composition comprises CD4+ and CD8+ T cells and the ratio of CD4+ to CD8+ T cells is from at or about 1:3 to 3:1, optionally at or about 1:2 to 2:1, optionally at or about 1:1.

225. A method of producing an antibody or antigen-binding portion that specifically binds to BAFF-R, comprising culturing the host cell of embodiment 215 under suitable conditions, and obtaining the product expressed by the host cell.

226. A method for preparing a BAFF-R-targeting drug, an anti-BAFF-R antibody-drug conjugate (ADC), a multifunctional anti-BAFF-R antibody, a reagent for diagnosing a tumor expressing BAFF-R, or an anti-BAFF-R chimeric antigen receptor (CAR) modified immune cell, wherein the method comprises providing the antibody or antigen-binding portion of any of embodiments 149-193 and incorporating said antibody or antigen-binding portion into the BAFF-R-targeting drug, the anti-BAFF-R ADC, the multifunctional anti-BAFF-R antibody, the reagent for diagnosing a tumor expressing BAFF-R, or the anti-BAFF-R chimeric antigen receptor (CAR) modified immune cell.

227. A method of treatment, comprising administering the cell of any one of embodiments 214-220 or the composition of any one of embodiments 221-224 to a subject having a disease or disorder associated with BAFF-R.

228. The cell of any one of embodiments 214-220 or the composition of any one of embodiments 221-224 for use in treating a disease or disorder associated with BAFF-R.

229. Use of the cell of any one of embodiments 214-220 or the composition of any one of embodiments 221-224 for the manufacture of a medicament for treating a disease or disorder associated with BAFF-R.

230. Use of the cell of any one of embodiments 214-220 or the composition of any one of embodiments 221-224 for the treatment of a disease or disorder associated with BAFF-R.

231. A method of treatment, comprising administering the antibody or antigen-binding portion thereof of any of embodiments 149-193, the pharmaceutical composition of embodiment 194, the CAR of any one of embodiments 195-1202, the conjugate of embodiment 203 or embodiment 204, the nucleic acid of embodiment 205, the polynucleotide of any one of embodiments 206-209, or the vector of any one of embodiments 210-213 to a subject having a disease or disorder associated with BAFF-R.

232. The anti-BAFF-R antibody or antigen-binding portion thereof of any of embodiments 149-193, the pharmaceutical composition of embodiment 194, the CAR of any one of embodiments 195-202, the conjugate of embodiment 203 or embodiment 204, the nucleic acid of embodiment 205, the polynucleotide of any one of embodiments 206-209, or the vector of any one of embodiments 210-213 for use in treating a disease or disorder associated with BAFF-R.

233. Use of the anti-BAFF-R antibody or antigen-binding portion thereof of any one of embodiments 149-193, the pharmaceutical composition of embodiment 194, the CAR of any one of embodiments 195-202, the conjugate of embodiment 203 or embodiment 204, the nucleic acid of embodiment 205, the polynucleotide of any one of embodiments 206-209, or the vector of any one of embodiments 210-213 for the manufacture of a medicament for treating a disease or disorder associated with BAFF-R.

234. Use of the antibody or antigen-binding portion thereof of any one of embodiments 149-193, the pharmaceutical composition of embodiment 194, the CAR of any one of embodiments 195-202, the conjugate of embodiment 203 or embodiment 204, the nucleic acid of embodiment 205, the polynucleotide of any one of embodiments 206-209, or the vector of any one of embodiments 210-213 for the treatment of a disease or disorder associated with BAFF-R.

235. The method, the cell, composition, antibody or antigen-binding portion thereof, conjugate, polynucleotide, or vector for use or the use of any of embodiments 225-234, wherein the disease or disorder associated with BAFF-R is a cancer.

236. The method, the cell, composition, antibody or antigen-binding portion thereof, conjugate, polynucleotide, or vector for use or the use of embodiment 235, wherein the cancer is a BAFF-R-expressing cancer.

237. A kit comprising the antibody or antigen-binding portion thereof of any of embodiments 149-193, the pharmaceutical composition of embodiment 194, the CAR of any one of embodiments 195-202, the conjugate of embodiment 203 or embodiment 204, the nucleic acid of embodiment 205, the polynucleotide of any one of embodiments 206-209, or the vector of any one of embodiments 210-213, the cell of any one of embodiments 214-220 or the composition of any of embodiments 221-224, and instructions for use, optionally wherein the instructions are for administering the antibody or antigen-binding portion thereof, the conjugate, the cell, or the composition, optionally in accord with the method, the cell, composition, antibody or antigen-binding portion thereof, conjugate, polynucleotide, or vector for use or the use of any of embodiments 225-236.

238. An article of manufacture comprising the antibody or antigen-binding portion thereof of any one of embodiments 149-193, the pharmaceutical composition of embodiment 194, the CAR of any one of embodiments 195-202, the conjugate of embodiment 203 or embodiment 204, the nucleic acid of embodiment 205, the polynucleotide of any one of embodiments 206-209, or the vector of any one of embodiments 210-213, the cell of any one of embodiments 214-220, the composition of any one of embodiments 221-224, or the kit of embodiment 237.

### IX. EXAMPLES

The following examples are included for illustrative purposes only and are not intended to limit the scope of the invention.

### Example 1: Generation of Antibodies to BAFF Receptor (BAFF-R)

Mice that were genetically modified to produce antibodies containing fully human antibody variable regions were immunized with recombinant extracellular binding domain (ECD) of human BAFF-R. The spleen, lymph nodes and bone marrow were harvested, and BAFF-R specific memory B cells and plasma cells were enriched by immunoaffinity-based selection. Individual antibody variable heavy (V_{H}) and variable light (V_{L}) chain pairs were sequenced from cell populations from enriched fractions.

The V_{H} and V_{L} sequences were reformatted as single chain variable antibodies (scFv) in different orientations (V_{L}-V_{H} & V_{H}-V_{L}) and with a linker sequence set forth in SEQ ID NO: 58 between the V_{H} and V_{L}. About 270 scFv binders were then used for subsequent analysis. The antibodies were formatted as part of a chimeric antigen receptor and assessed for binding and activity as described in Examples below.

### Example 2: Generation and Assessment of Anti-BAFF-R Chimeric Antigen Receptor (CAR)

The synthesized anti-BAFF-R-encoding sequences were cloned into an exemplary polynucleotide construct to generate candidate polynucleotides encoding chimeric antigen receptors (CARs) containing the candidate BAFF-R scFv binders as the antigen-binding domains. Specifically, the polynucleotide CAR constructs contained nucleic acid sequences encoding a signal peptide; a candidate anti-BAFF-R scFv binder; a spacer: either a spacer containing a modified IgG4/IgG2-hinge CH2-CH3 (SEQ ID NO:53) or a spacer containing a modified IgG4 hinge region (SEQ ID NO:52); a human CD28 transmembrane domain (SEQ ID NO: 55); a human 4-1BB intracellular signaling region (SEQ ID NO: 56); and a human CD3-zeta intracellular signaling region (SEQ ID NO: 57). Polynucleotides encoding the CARs were cloned into a lentiviral expression vector for transduction of T cells.

Viral preparations encoding the various candidate anti-BAFF-R CARs were individually introduced by transduction into a Jurkat T cell line containing a Nur77 knock-in reporter (see, e.g., WO 2019/089982). The Nur77 knock-in cell line contained nucleic acid sequences encoding a reporter molecule (e.g., a red fluorescent protein) knocked-in at the endogenous Nur77 locus, which is an immediate-early response gene induced by stimulation of signal from the T cell receptor and/or via molecules containing immunoreceptor tyrosine-based activation motif (ITAM). The Jurkat reporter cells were assessed for cell surface expression of the CAR, and antigen-dependent and antigen-independent signaling.

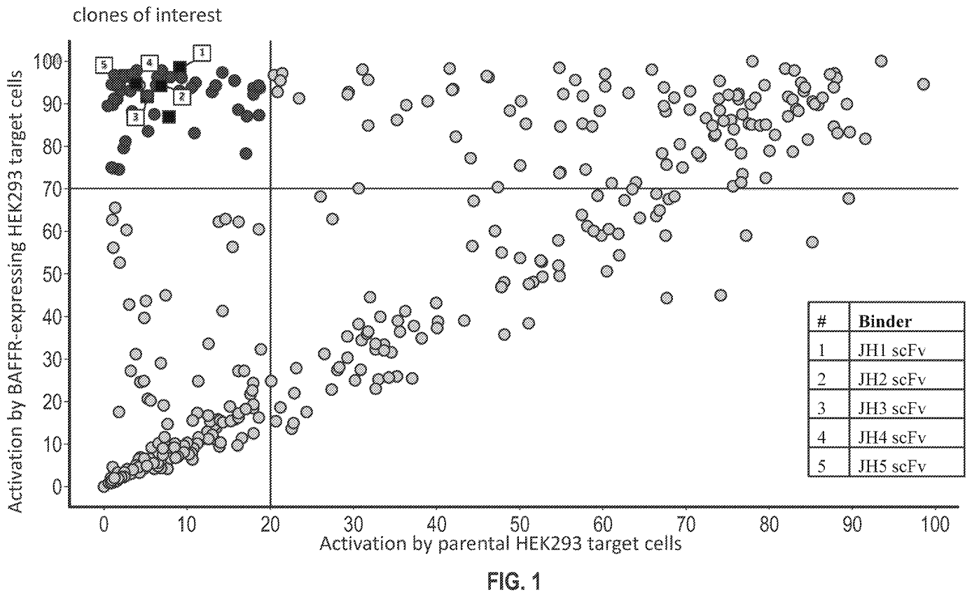

After transduction, the Jurkat reporter cells were evaluated for the expression of red fluorescent protein after co-culture with HEK293 target cells expressing BAFF-R or parental HEK293 target cells. FIG. 1 shows the results of the BAFF-R CAR library screen. Jurkat reporter cells transduced with candidate anti-BAFF-R CARs exhibited varying levels of antigen-specific activity, and certain BAFF-R CARs also exhibited tonic signaling as evidenced by detection of reporter cell activation in parental cells lacking BAFF-R antigen. About 60 anti-BAFF-R CARs each incorporating a different anti-BAFF-R scFv were selected that exhibited high BAFF-R antigen-dependent activity but low tonic signaling (selected CARs depicted in the dashed box of the upper left corner).

### Example 3: Design and Generation of Anti-BAFF-R/Anti-CD19 (BAFFR×CD19) Tandem CARs

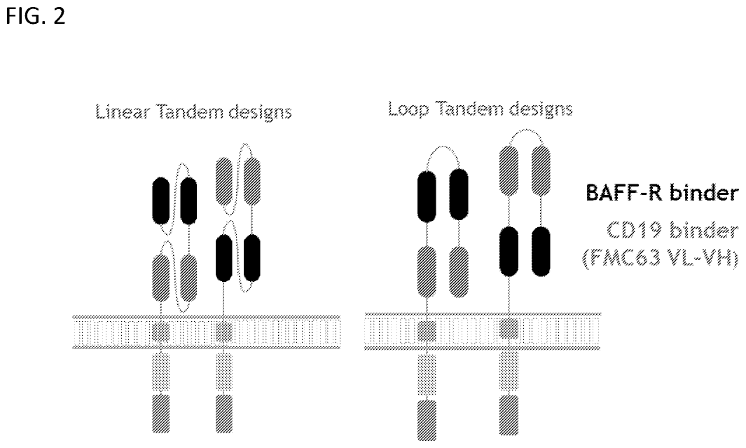

Exemplary tandem CARs were engineered, each incorporating an extracellular binding domain having (1) a BAFF-R targeted binding domain comprising the variable heavy chain (V_{H}) and the variable light chain (V_{L}) sequences of BAFF-R-targeted scFv binders identified in Example 2, and (2) a CD19-targeted binding domain comprising the variable heavy chain (V_{H}) and the variable light chain (V_{L}) sequences derived from anti-CD19 antibody FMC63 (V_{H} set forth in SEQ ID NO: 41 and V_{L} set forth in SEQ ID NO: 42). The extracellular binding domains were generated to have various structural configurations, including different combinations of: arrangement of the two binding domains in either linear tandem or loop tandem format; order of the V_{H} and the V_{L} of each binding domain, from N- to C-terminus; linkers between the V_{H} and the V_{L} of the same binding domain (“intra-domain linker,” e.g., SEQ ID NO: 58 or 59); linkers between the different binding domains (“inter-domain linker,” e.g. SEQ ID NO: 60 or 61); and arrangement of either the BAFF-R-targeted binding domain or the CD19-targeted binding domain proximal to the cellular membrane. As depicted in FIG. 2, the different binders were oriented in either a linear tandem design (Table E2B) or a loop tandem design (Table E2A) with either of the BAFF-R scFv binder or the CD19 scFv binder being proximal to the membrane.

Each generated tandem CAR construct contained the extracellular binding domain having both the BAFF-R and CD19-targeted binding domains; one of two different length immunoglobulin-derived spacer domains [short (hinge only, 12aa; SEQ ID NO: 52) or long (hinge-CH2-CH3, 228aa; SEQ ID NO: 53, encoded by the sequence set forth in SEQ ID NO: 54) with CH2 modifications to limit Fc receptor binding]; a human CD28-derived transmembrane domain (SEQ ID NO: 55); a human 4-1BB-derived intracellular signaling domain (SEQ ID NO: 56); and a human CD3zeta-derived intracellular signaling domain (SEQ ID NO: 57). Additionally, varying intra-domain and inter-domain linkers were used in the construction of the library. Polynucleotides encoding the tandem CARs were cloned into a lentiviral expression vector for transduction of T cells.

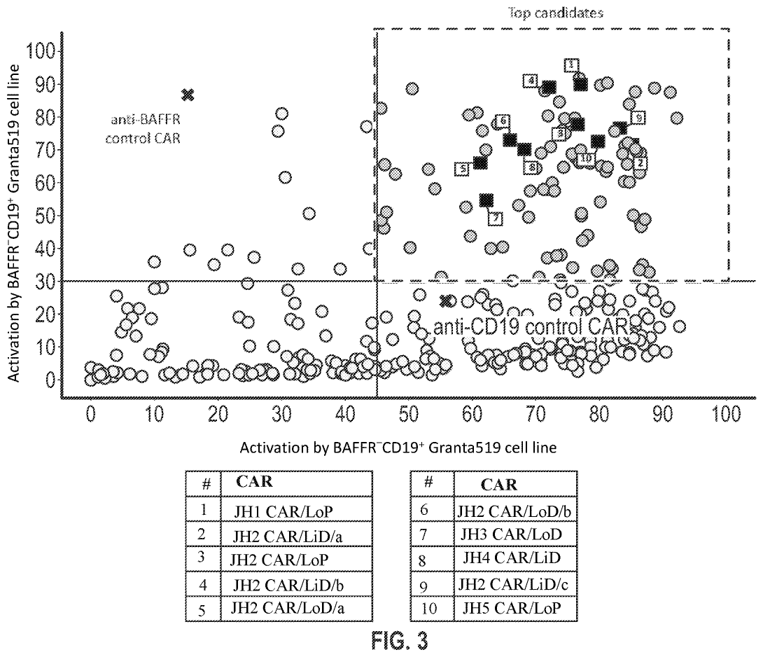

The tandem CARs were screened using the Jurkat reporter assay as described in Example 2. After transduction, the Jurkat reporter cells were evaluated for the expression of red fluorescent protein after co-culture with both Granta cells containing BAFF-R KO (BAFF-R-CD19+ Granta B cells) and Granta cells containing CD19 KO (BAFF-R+CD19-Granta B cells). To evaluate antigen-independent activity, CAR-transduced reporter cells were cultured alone in the absence of antigen-expressing target cells. The CAR-transduced reporter cells were assessed for level of antigen-independent (basal) signaling in the absence of target antigen, and inducible CAR signaling in the presence of target cells expressing CD19, BAFF-R, or both. The transduced reporter cells were also assessed for expression of the tandem CAR. Candidate tandem CARs were selected that expressed well, exhibited low level of basal signaling and exhibited high antigen-dependent CAR signaling. Results for exemplary CARs are shown in FIG. 3, which shows the ability of the tandem CARs to be stimulated by BAFF-R-CD19+ cells, BAFF-R+CD19− cells, neither group of cells, or both groups of cells (only candidates with low basal signaling are shown).

Based upon these factors, about 100 candidate tandem CARs (depicted in FIG. 3, upper right corner) were selected for further analysis as described in Examples below.

### Example 4: Assessment of CAR Expression and Function in Primary T Cells

The expression and function of the tandem CARs selected in Example 3 were assessed in primary human T cells.

CD4+ and CD8+ T cells were isolated by immunoaffinity-based enrichment from leukapheresis samples of one human donor. Isolated CD4+ and CD8+ T cells were mixed at approximately 1:1 ratio, and the combined cell population was stimulated with anti-CD3/anti-CD28 antibody stimulation reagent in the presence of recombinant IL-2, IL-7, and IL-15, transduced with lentiviral preparations encoding a selected tandem CAR, and cultivated under conditions for expansion. As controls, primary T cells also were similarly stimulated and transduced with lentiviral preparations encoding a reference anti-CD19 CAR or a reference anti-BAFF CAR.

The primary T cells expressing one of the candidate CARs were co-cultured with Granta cells expressing both CD19 and BAFF-R (Granta parental cells) or were cultured with Granta cells that were engineered to eliminate expression of one of the antigens by knock out (KO): Granta CD19 KO (BAFF-R+) and Granta BAFF-R KO (CD19+). The Granta cells also were labeled with NucLight Red (NLR) to monitor cytolytic cell killing via analysis of red fluorescent signal over time on an IncuCyte® instrument. To assess antigen-dependent CAR activity, co-cultures were assessed for cytolytic activity of the Granta target cells based on AUC measurements by the Incucyte assay and cytokine response by the CAR T cell by measurement of IFN-γ, IL-2, and TNF-α in culture supernatant.

Four experimental batches were run collectively testing thirty (30) selected CARs for cytolytic killing and cytokine production using the above methods. The Batch IDs of the ten (10) selected CARs are listed below in Table E2.

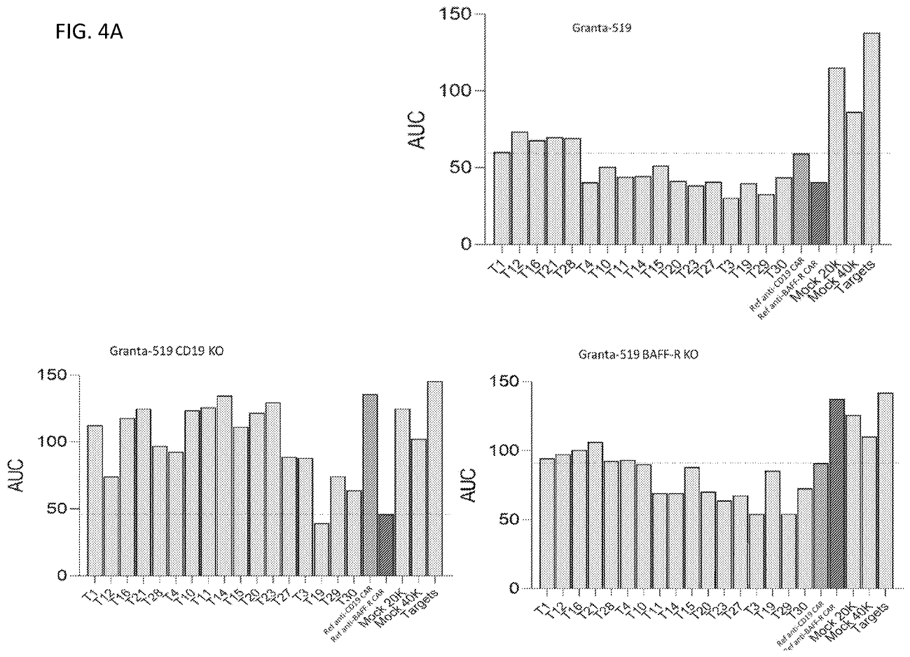

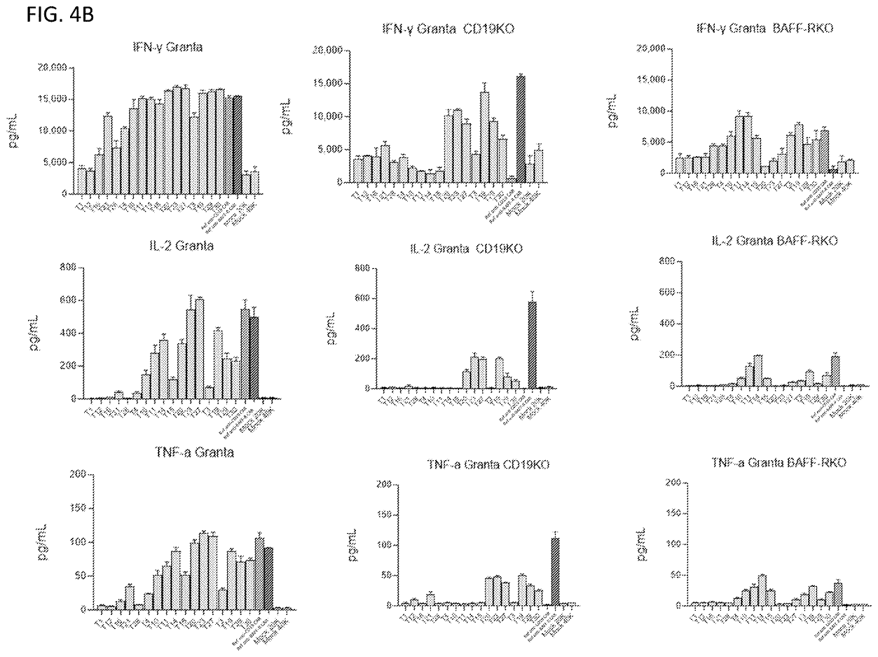

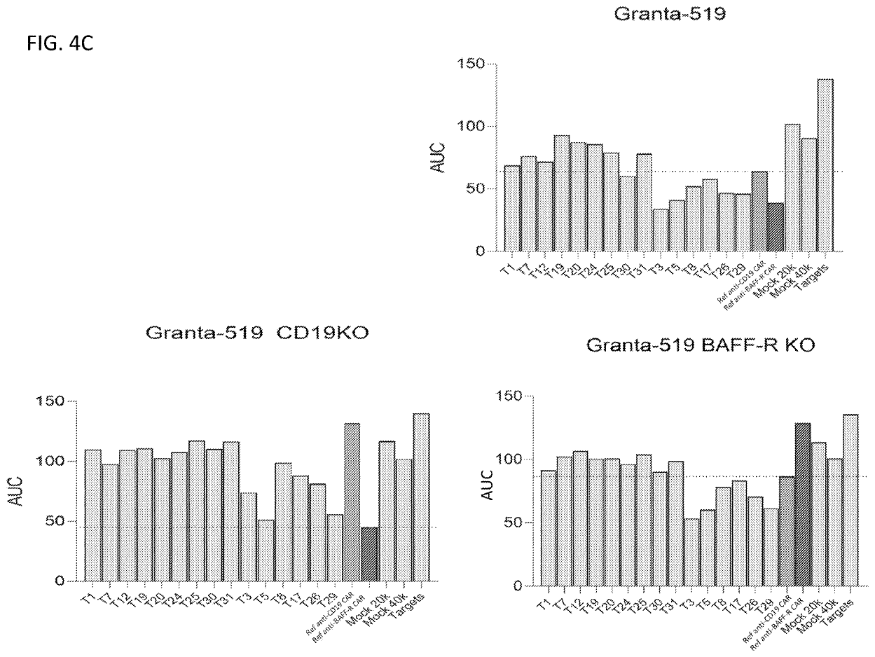

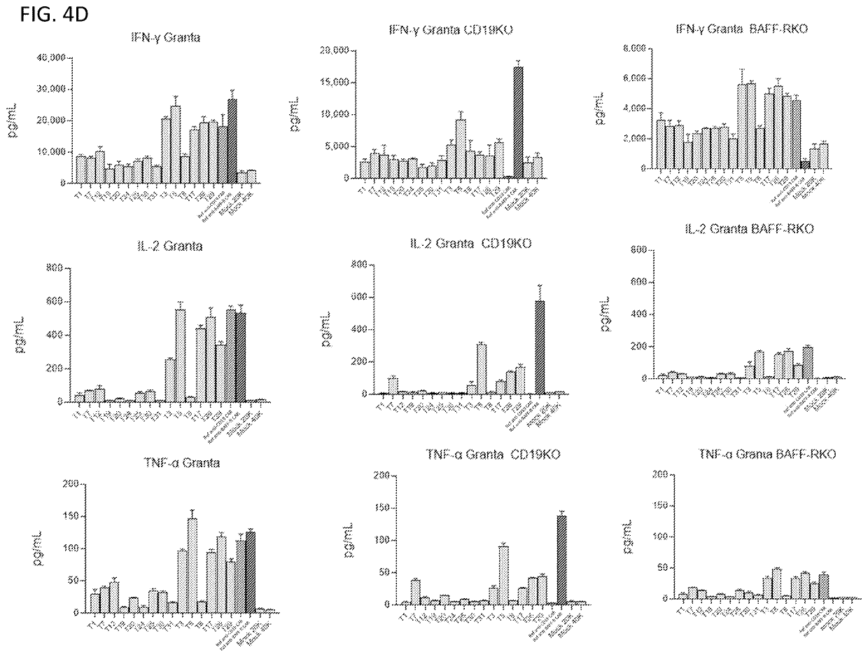

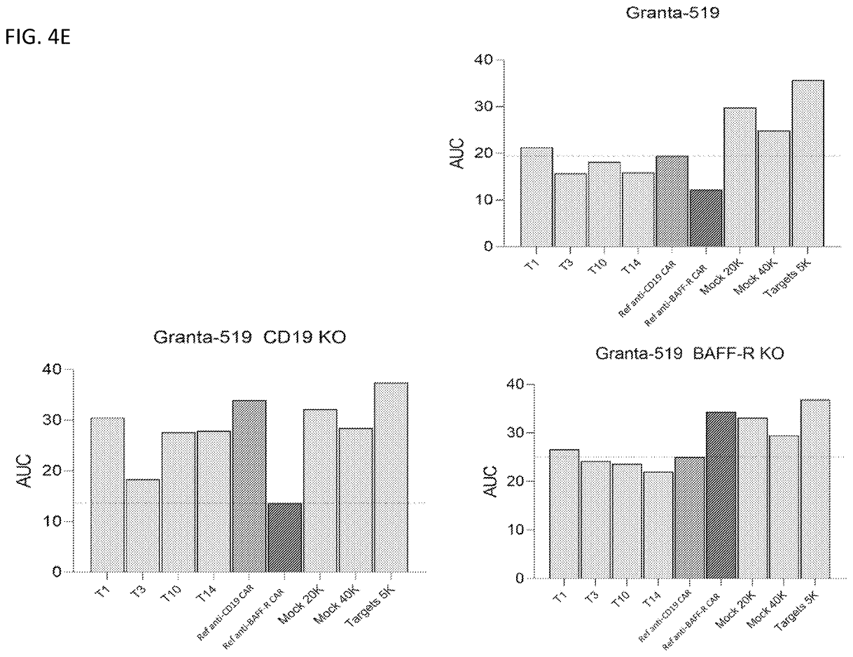

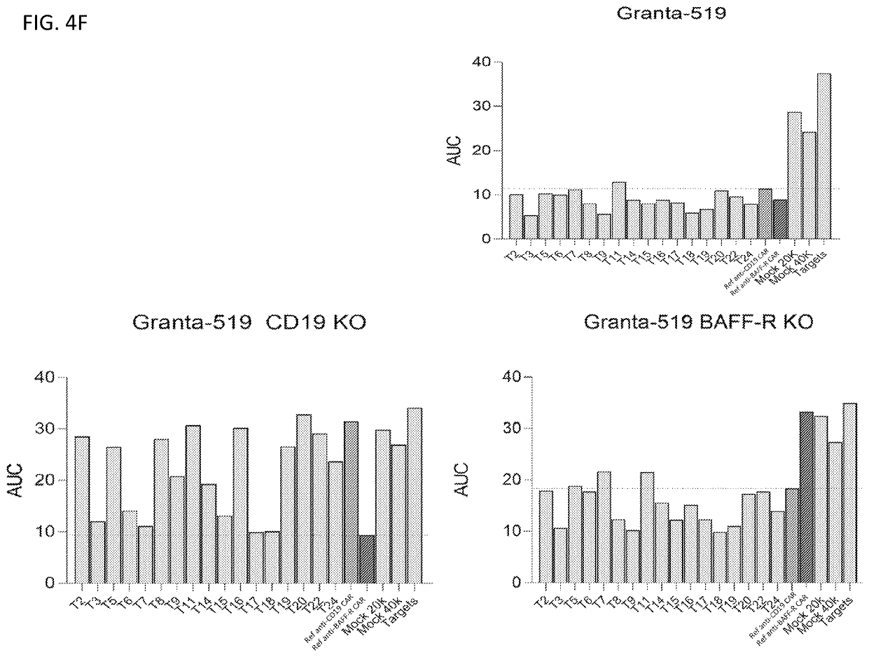

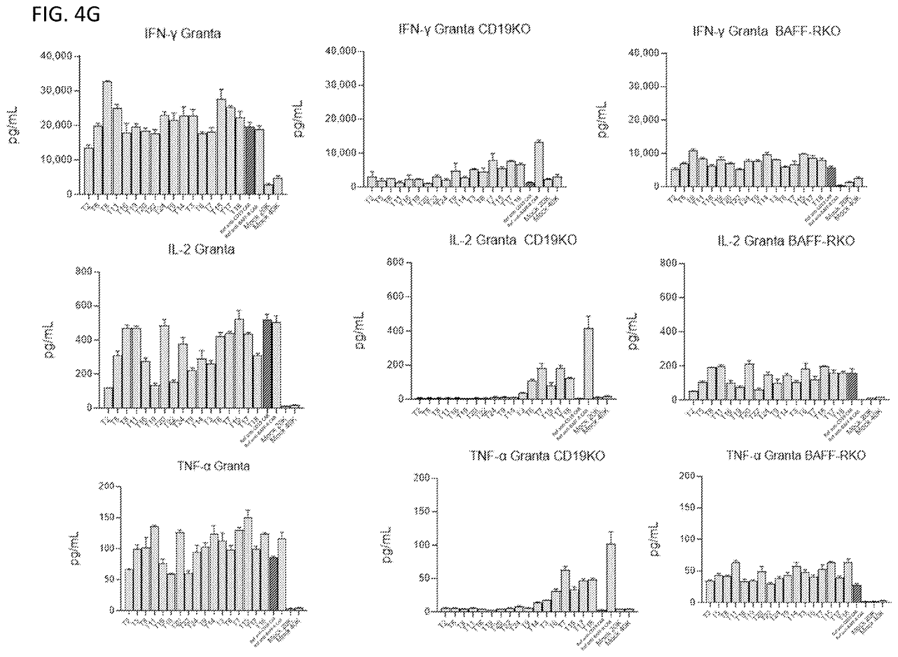

Results for cell killing capability is shown: Batch 1: FIG. 4A; Batch 2: FIG. 4C; Batch 3: FIG. 4E; Batch 4: FIG. 4F. Results for cytokine production in the supernatant is shown: Batch 1: FIG. 4B; Batch 2: FIG. 4D; Batch 4: FIG. 4G). The anti-CD19 and anti-BAFF-R reference CARs showed very little cytolytic killing or cytokine production when co-cultured with the CD19 KO and BAFF-R KO cultures respectively, while candidate CARs yielded varying levels of cytolytic killing and cytokine production.

Tandem CARs were selected that exhibited among the highest transduction efficiency, and antigen-dependent CAR activity for both target antigens expressed by the Granta target cells.

Ten (10) tandem CARs were selected for further in vitro testing. Five unique BAFF-R targeting domains derived from sequences from five unique scFvs identified in Example 2 were featured among these top 10 tandem CARs. The 10 tandem CARs include both linear and loop tandem configurations as well as configurations in which the CD19 binder is in either the membrane proximal or membrane distal position. The sequences of the CDRs and the scFvs of these exemplary BAFF-R scFv binders are presented in Table E1 below.

TABLE E1
Sequence identifier (SEQ ID NO:) for BAFF-R Binders

| BAFF-R | SEQ ID NO: | | | | | | | | | |
|---|---|---|---|---|---|---|---|---|---|---|
| Binder | V_{H} | CDR-H1 | CDR-H2 | CDR-H3 | V_{L} | CDR-L1 | CDR-L2 | CDR-L3 | scFv | Linker |
| JH1 | 1 | 16 | 17 | 18 | 2 | 19 | 20 | 21 | 11 | 58 |
| JH2 | 3 | 22 | 23 | 24 | 4 | 25 | 26 | 27 | 12 | 58 |
| JH3 | 5 | 28 | 29 | 30 | 6 | 31 | 26 | 27 | 13 | 58 |
| JH4 | 7 | 22 | 32 | 24 | 8 | 33 | 26 | 34 | 14 | 58 |
| JH5 | 9 | 35 | 36 | 37 | 10 | 38 | 39 | 40 | 15 | 58 |

The construction of the bispecific tandem targeting domain of the ten selected CARs is shown in Table E2A and E2B below.

TABLE E2A
Targeting domain from amino to carboxy terminus (SEQ ID NO) - Tandem Loop (Lo)

| | | Inter- | | Intra- | | Inter- | | Batch |
|---|---|---|---|---|---|---|---|---|
| Tandem | | domain | Domain | domain | Domain | domain | Domain | CAR |
| CAR | Domain 1 | Linker | 2 | Linker | 3 | Linker | 4 | ID |
| JH1 | CD19 VL | G4S | BAFF-R | (G4S)x3 | BAFF-R | G4S | CD19 VH | Batch 1 |
| CAR/LoP | (42) | (60) | VH | (58) | VL | (60) | (41) | T19 |
| | | | (1) | | (2) | | | |
| JH2 | CD19 VL | G4S | BAFF-R | (G4S)x3 | BAFF-R | G4S | CD19 VH | Batch 2 |
| CAR/LoP | (42) | (60) | VH | (58) | VL | (60) | (41) | T29 |
| | | | (3) | | (4) | | | |
| JH2 | BAFF-R | (G4S)x2 | CD19 | Whitlow | CD19 VH | (G4S)x2 | BAFF-R | Batch 4 |
| CAR/LoD/ | VL | (61) | VL | (59) | (41) | (61) | VH | T15 |
| a | (4) | | (42) | | | | (3) | |
| JH2 | BAFF-R | G4S | CD19 | Whitlow | CD19 VH | G4S | BAFF-R | Batch 4 |
| CAR/LoD/ | VH | (60) | VL | (59) | (41) | (60) | VL | T17 |
| b | (3) | | (42) | | | | (4) | |
| JH3 | BAFF-R | (G4S)x2 | CD19 | Whitlow | CD19 VH | (G4S)x2 | BAFF-R | Batch 4 |
| CAR/LoD | VL | (61) | VL | (59) | (41) | (61) | VH | T18 |
| | (6) | | (42) | | | | (5) | |
| JH5 | CD19 VL | G4S | BAFF-R | (G4S)x3 | BAFF-R | G4S | CD19 VH | Batch 2 |
| CAR/LoP | (42) | (60) | VH | (58) | VL | (60) | (41) | T17 |
| | | | (9) | | (10) | | | |

TABLE E2B
Targeting domain from amino to carboxy terminus (SEQ ID NO) - Tandem Linear (Li)

| | | Intra- | | Inter- | | Intra- | | Batch |
|---|---|---|---|---|---|---|---|---|
| Tandem | | domain | Domain | domain | Domain | domain | Domain | CAR |
| CAR | Domain 1 | Linker | 2 | Linker | 3 | Linker | 4 | ID |
| JH2 | CD19 VL | Whitlow | CD19 VH | G4S | BAFF-R | (G4S)x3 | BAFF-R | Batch 2 |
| CAR/LiD/ | (42) | (59) | (41) | (60) | VH | (58) | VL | T5 |
| a | | | | | (3) | | (4) | |
| JH2 | CD19 VL | Whitlow | CD19 VH | G4S | BAFF-R | (G4S)x3 | BAFF-R | Batch 4 |
| CAR/LiD/ | (42) | (59) | (41) | (60) | VL | (58) | VH | T6 |
| b | | | | | (4) | | (3) | |
| JH4 | CD19 VL | Whitlow | CD19 VH | G4S | BAFF-R | (G4S)x3 | BAFF-R | Batch 4 |
| CAR/LiD | (42) | (59) | (41) | (60) | VH | (58) | VL | T3 |
| | | | | | (7) | | (8) | |
| JH2 | CD19 VL | Whitlow | CD19 VH | G4S4 | BAFF-R | (G4S)x3 | BAFF-R | Batch 2 |
| CAR/LiD/ | (42) | (59) | (41) | (62) | VL | (58) | VH | T26 |
| c | | | | | (4) | | (3) | |

### Example 5: Cytolytic Activity and Cytokine Production of Selected CAR T Cells in Response to B Cell Cancer Lines

Antigen-dependent activity of the CARs selected as described in Example 4 was examined against multiple B cell cancer lines. CD4+ and CD8+ T cells isolated by immunoaffinity-based enrichment from leukapheresis samples from a human donor subject were stimulated and transduced with lentiviral preparations encoding one of the ten tandem anti-BAFF-R/anti-CD19 bispecific CARs. T cells also were transduced with a reference anti-CD19 CAR or a reference anti-BAFF-R CAR as a control.

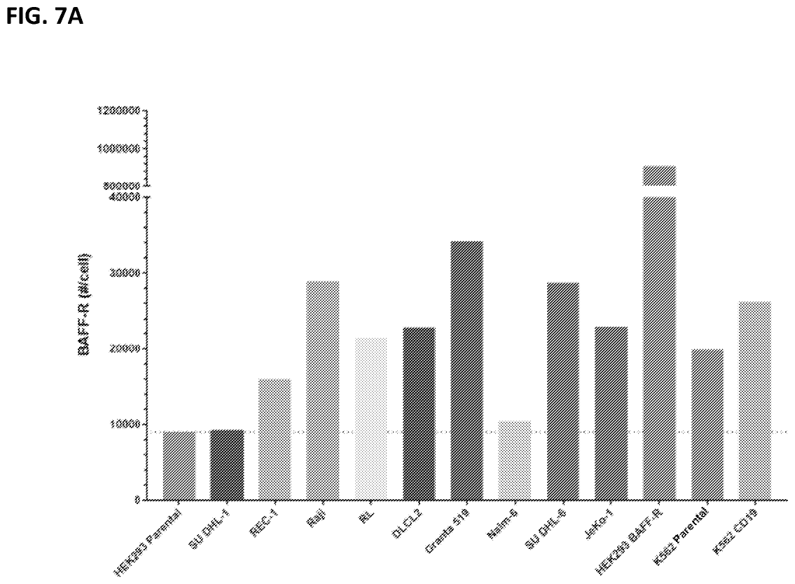

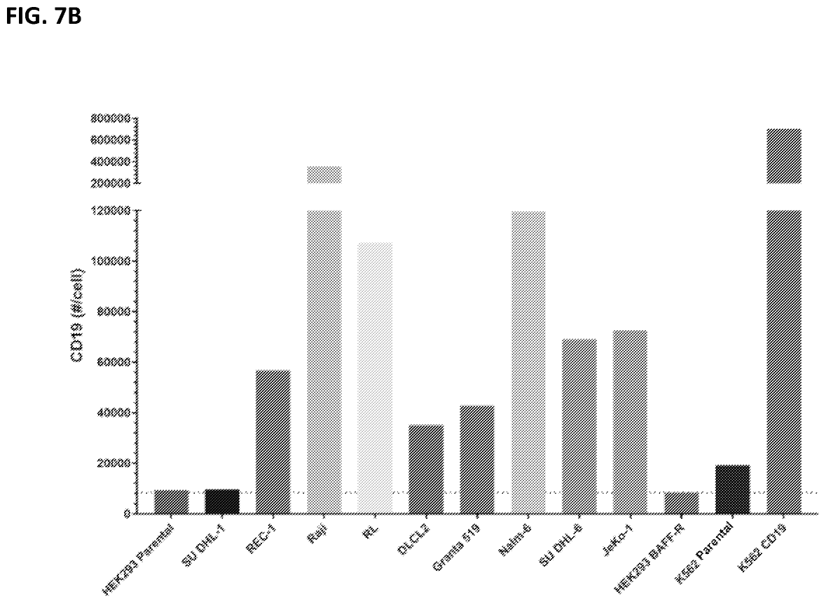

Engineered CAR-T cells were co-cultured in triplicate with Granta-519 target cell B cell cancer lines in vitro at an effector: target (E:T) ratio of 1:2. The Granta-519 target cell B cell cancer lines included: Granta cells (either (a) parental, CD19+& BAFF-R+; (b) Granta CD19 KO, CD19− & BAFF-R+; or (c) Granta BAFF-R KO, CD19+& BAFF-R−). All target cells were labeled with NucLight Red (NLR) to monitor cytolytic cell killing. Expression of BAFF-R and CD19 in various cell lines including Granta cell lines is shown in FIGS. 7A and 7B.

To assess antigen-dependent CAR activity, co-cultures were assessed for cytolytic activity of the target cells and for cytokine production in the supernatant. Cytolytic activity was assessed by measuring the loss of viable target cells over a period of between 0 and 108 hours, as determined by changes in red fluorescent signal. The production of cytokines IFN-Y, TNF-α and IL-2 in the supernatant was also assessed after 108 hours of co-culture. As controls, cultures of target cells only and co-cultures of mock cells (not expressing a CAR) with the target cells were assessed.

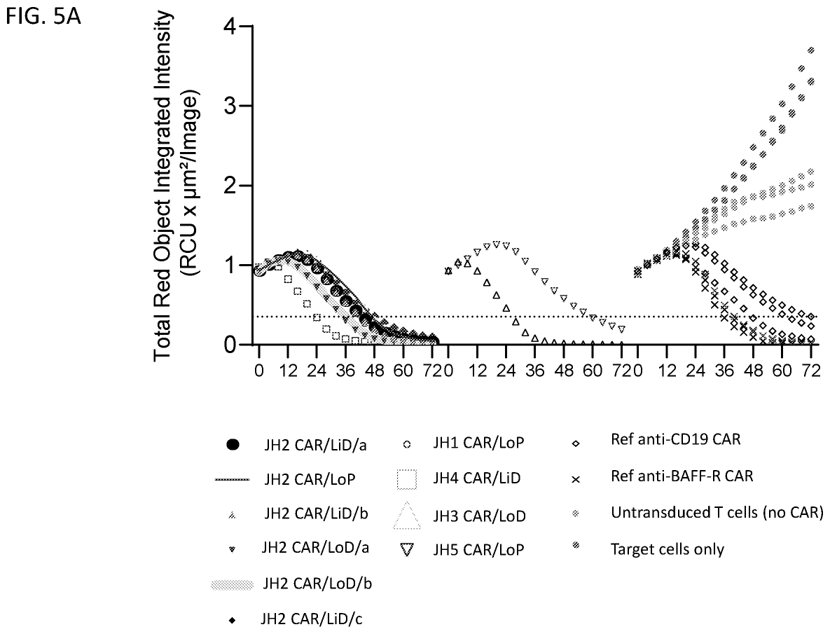

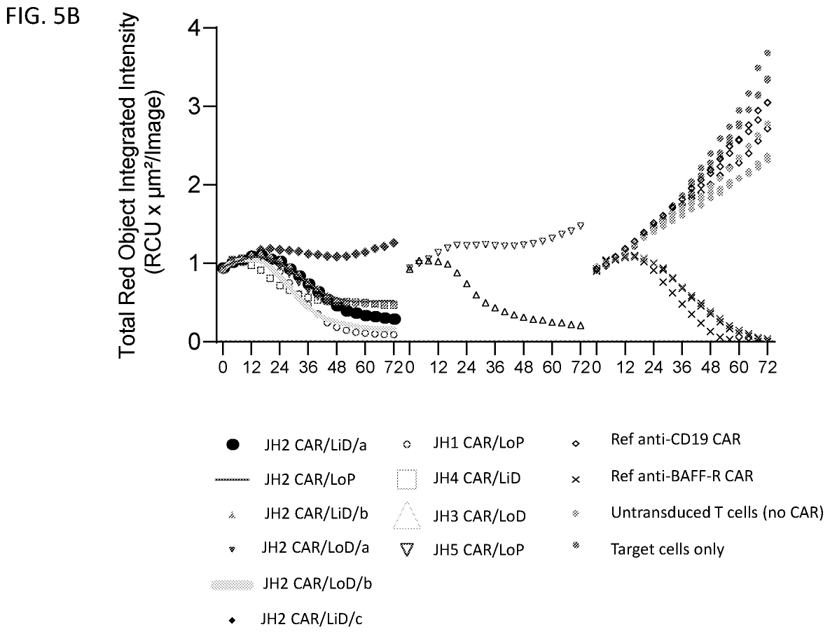

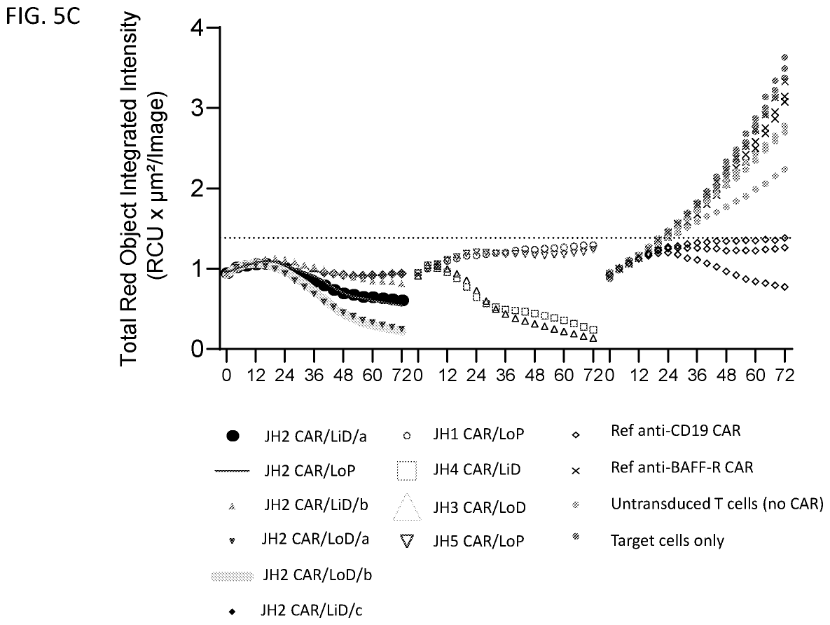

As shown in FIGS. 5A-5C, all tested tandem anti-BAFF-R/anti-CD19 CARs were functionally active, including against target cells expressing only a single target antigen, either CD19 or BAFF-R antigens (FIGS. 5B and 5C), as well as against target cells expressing both target antigens (FIG. 5A). This indicates that the CD19 binding domain (FMC63) and the various BAFF-R binding domains were able to function independently within the tandem CARs, including linear and loop tandem designs as well as configurations in which the CD19 binder is in the membrane proximal and membrane distal position of the tandem CAR. All of the candidate tandem CARs tested showed robust in vitro cytotoxic activity against the cancer lines. The cytotoxic killing activity of several tandem anti-CD19/anti-BAFF-R CARs was similar to or greater than the single targeting anti-CD19 or anti-BAFF-R CAR controls against respective target cells expressing CD19 or BAFF-R, respectively.

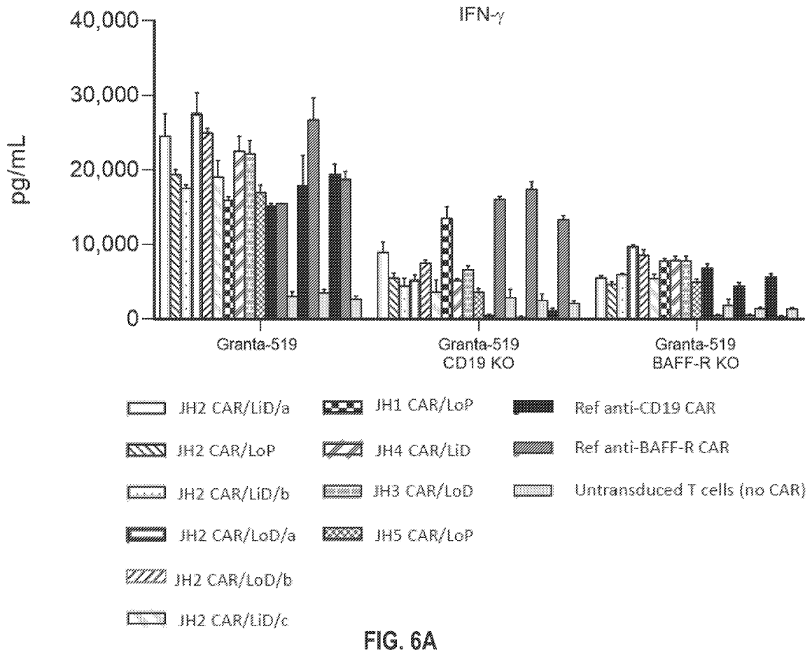

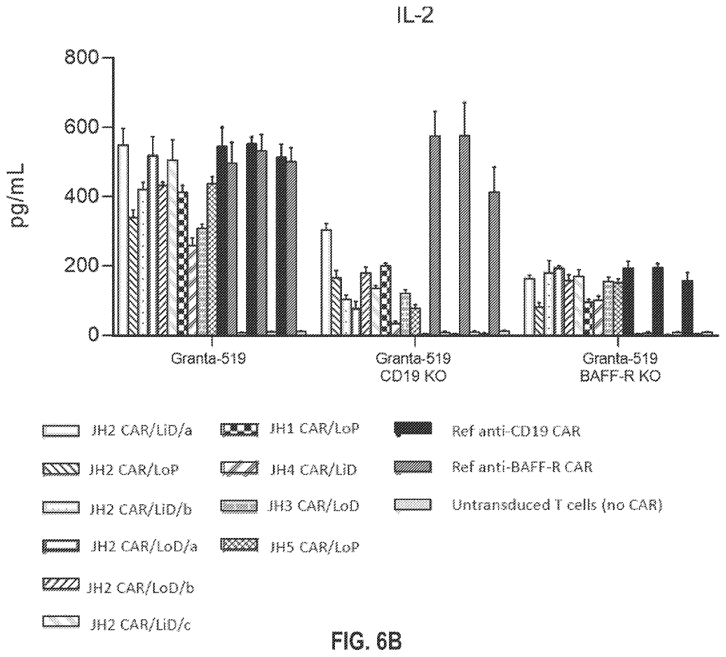

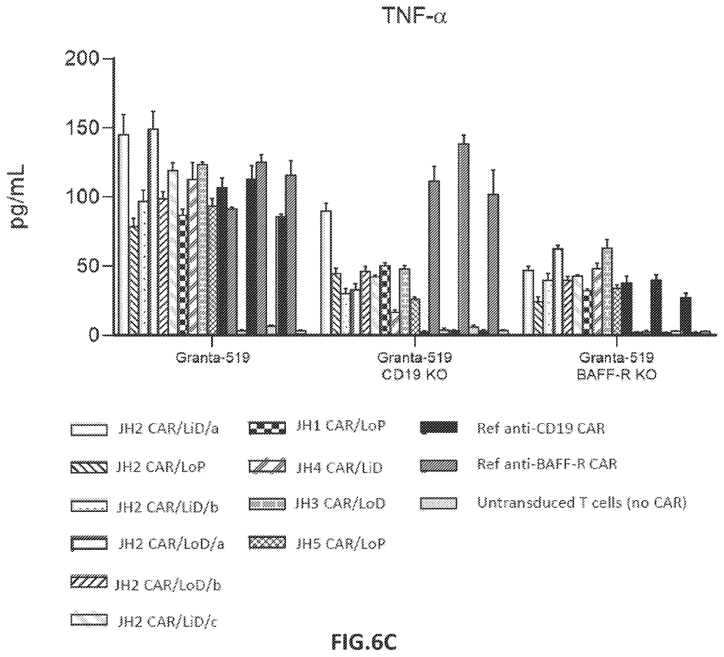

As shown in FIGS. 6A-6C, the anti-BAFF-R/anti-CD19 tandem CARs produced varying levels of three cytokines analyzed in the different cell assays. All ten tandem CARs stimulated production of IFN-γ (FIG. 6A), IL-2 (FIG. 6B) and TNF-α (FIG. 6C) when exposed to Granta target cells with both antigens present or when missing either the CD19 or BAFF-R antigen, although the level of cytokine production differed among the different tandem CARs. In contrast, T cells engineered with the anti-CD19 CAR control or the anti-BAFF-R CAR control did not exhibit cytokine production in Granta cells that were CD19− (Granta CD19 KO) or BAFF-R− (Granta BAFF-R KO), respectively. This suggests the tandems may be less susceptible to variation of targeted antigen levels compared to single-target CAR-T comparators. Further, several tandem anti-CD19/anti-BAFFR tandem CARs exhibited similar ability to stimulate production of cytokines in cells that were positive for CD19 or BAFF-R, or both antigens, as the single targeting anti-CD19 or anti-BAFF-R CAR controls.

### Example 6: Effect of Soluble BAFF on Selected Bispecific CAR T Cells

The effect of soluble BAFF on seven (7) selected anti-CD19/anti-BAFF-R bispecific CARs was assessed. JeKo-1 target cells (clone 4.6) (BAFF-R^{low} CD19⁺) were preincubated with anti-CD19 clone FMC63 scFv; soluble BAFF (sBAFF); or both anti-CD19 clone FMC63 scFv and sBAFF. All proteins were added to a final concentration of 20 μg/mL. The anti-CD19 FMC63 scFv was used to block binding of the anti-CD19 arm of the CAR to the cellular CD19 antigen while the soluble BAFF (sBAFF) acted to block the anti-BAFF-R arm of the CAR to the cellular BAFF-R antigen.

25,000 CAR T cells engineered with a bispecific tandem CAR (Table E1) were co-cultured with 25,000 JeKo-1 (BAFF-R^{low} CD19⁺) target cells for 4.5 hours. Supernatant was collected and production of IFN-γ was determined using a Meso Scale Discovery (MSD) ELISA-based assay platform.

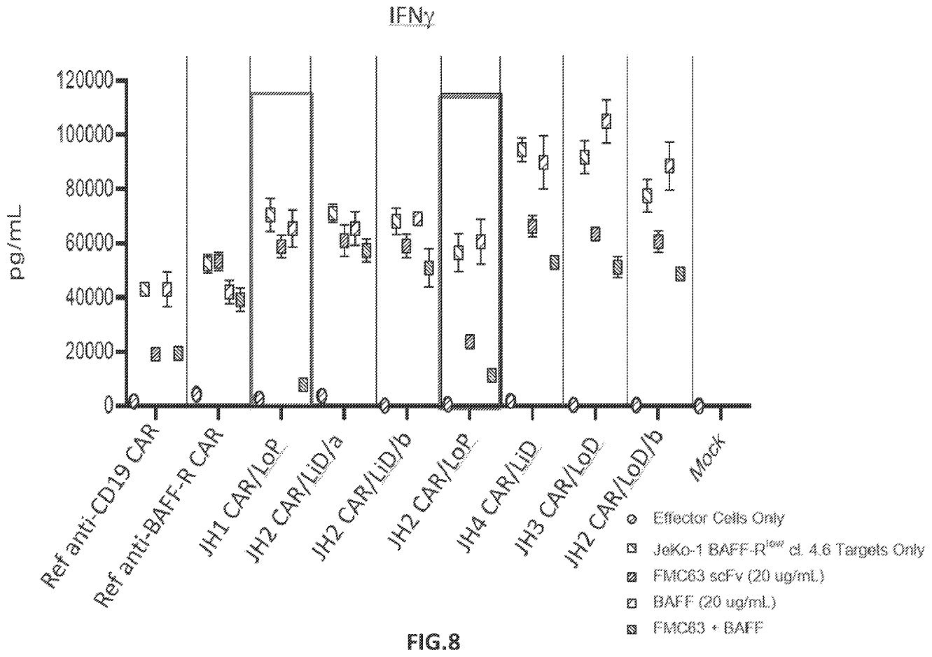

As shown in FIG. 8 and Table E3 below, two bispecific tandem CARs (JH1 CAR/LoP and JH2 CAR/LoP) show a greater than 80% decrease in IFN-γ production when the cells were cultured in the presence of soluble BAFF with anti-CD19 FMC63 scFv. These two bispecifics also showed a greater than 50% decrease in IFN-γ production when the cells were cultured with both anti-CD19 FMC63 scFv+sBAFF relative to cells cultured in the presence of anti-CD19 FMC63 scFv alone. Five bispecific CARs (JH2 CAR/LiD/a, JH2 CAR/LiD/b, JH4 CAR/LID, JH3 CAR/LOD, and JH2 CAR/LoD/b) showed no strong decrease (~20% or less) of IFN-γ production when the cells were cultured with soluble BAFF either alone or relative to cultures with anti-CD19 FMC63 scFv alone.

Results are summarized in Table E3 and FIG. 8.

TABLE E3
Effect of soluble BAFF on selected bispecific CARs

| | | | FMC63 scFv + |
|---|---|---|---|
| Bispecific CAR | FMC63 scFv | sBAFF | sBAFF |
| JH1 CAR/LoP | +/− | − | ++ |
| JH2 CAR/LiD/a | +/− | − | +/− |
| JH2 CAR/LoP | + | − | ++ |
| JH2 CAR/LiD/b | +/− | − | +/− |
| JH2 CAR/LoD/b | +/− | − | + |
| JH3 CAR/LoD | + | − | + |
| JH4 CAR/LiD | +/− | − | + |
| Anti-CD19 reference CAR | + | − | + |
| Anti-BAFF-R reference | − | +/− | +/− |
| CAR | | | |

| Symbol | Blocking Level | Inhibition relative to target-only co-culture |
|---|---|---|
| − | No inhibition | <10% |
| +/− | Low | 10-30% |
| + | Medium | 30-80% |
| ++ | High | >80% |

As shown in FIG. 8, the preincubation of the candidate CARs with anti-CD19 clone FMC63 scFv; soluble BAFF (sBAFF); or both resulted in varying effects on cytokine production of the CARs. Six CARs (JH2 CAR/LiD/a, JH2 CAR/LOP, JH2 CAR/LiD/b, JH4 CAR/LiD, JH3 CAR/LOD, and JH2 CAR/LoD/b) exhibited a decrease of IFN-γ of ~10% or less when both soluble proteins are present as compared to the inhibition from FMC63 scFv alone. When CAR JH1 CAR/LOP was cultured with both FMC63 scFv and soluble BAFF, IFN-γ production was decreased greater than 50% as compared to cells cultured with FMC63 scFv alone. This result thus indicates that the format of the tandem CAR, such as the orientation of the binding domain, can impact antigen-binding and ability of activity to be blocked or reduced by soluble antigens.

### Example 7: Therapeutic Effect of Selected Bispecific CAR T Cells In Vivo

The ability of selected anti-CD19/anti-BAFF-R bispecific CARs to lessen tumor burden was assessed.

Polynucleotides encoding each tandem CAR candidate were introduced into primary human T cells by homology-directed repair (HDR)-targeted integration at the site of a genetic disruption introduced at the endogenous gene locus that encodes the T cell receptor alpha (TCRα) chain (TRAC locus) by CRISPR/Cas mediated gene editing. Template polynucleotides were generated for targeted integration by HDR containing the CAR transgene flanked by 5′ and 3′ homology arms homologous to sequences surrounding the target integration site of the TRAC locus. The HDR template polynucleotide encoding each CAR flanked by the homology arms was cloned into an adeno-associated virus (AAV) for generating an AAV preparation for T cell transduction.

Primary human CD4+ and CD8+ T cells were isolated by immunoaffinity-based selection from human peripheral blood mononuclear cells (PBMCs) and were stimulated by culturing with an anti-CD3/anti-CD28 stimulatory reagent at 37° C. in media with recombinant cytokines. Stimulated cells were then electroporated with ribonucleoprotein (RNP) complexes containing Cas9 and TRAC-targeting guide RNA (gRNA) to introduce a genetic disruption at the endogenous TRAC locus by CRISPR/Cas-mediated gene editing. After electroporation, an AAV preparation containing the DNA sequence encoding the CAR-T or tandem CAR-T of interest and homology arms homologous to sequences surrounding the target integration site in order to facilitate homology-directed DNA repair was added to each well. After an overnight incubation for recovery, the T cells were cultured under conditions for expansion.

### A. Raji Model

NOD scid gamma (NSG) mice were intravenously injected with 5×10⁵ Raji cells (CD19+ human B lymphoblastoid cells isolated from Burkitt lymphoma) that were engineered to express firefly luciferase to enable detection and monitoring of tumor burden.

At 6 days post injection, mice were staged and divided into groups (n=8 mice/group). Mice were administered 3×10⁶ (high dose), 1×10⁶ (medium dose) or 3.33×10⁵ (low dose) of CD19×BAFF-R bispecific CAR-T cells (Donor 20787), or 3×10⁶ or 1×10⁶ mock cells as control, at Day 0. Mean bioluminescence imaging (BLI), body weight, and survival were assessed twice a week for up to approximately 50 days after administration of the engineered T cells.

### B. Nalm6 Model

NOD scid gamma (NSG) mice were intravenously injected with 5×10⁵ Nalm6 cells (CD19⁺ B cell Acute Lymphoblastic Leukemia cell line) that were engineered to express firefly luciferase to enable detection and monitoring of tumor burden. At 3 days post injection, mice were staged and divided into groups (n=8 mice/group). Mice were administered 1×10⁶ (high dose) or 3.33×10⁵ (low dose) CD19×BAFF-R bispecific CAR-T cells (Donor 20787), or 1×10⁶ mock cells as control, at Day 0. Mean bioluminescence imaging (BLI), body weight, and survival were assessed twice a week for up to approximately 50 days after administration of the engineered T cells.

### C. Results

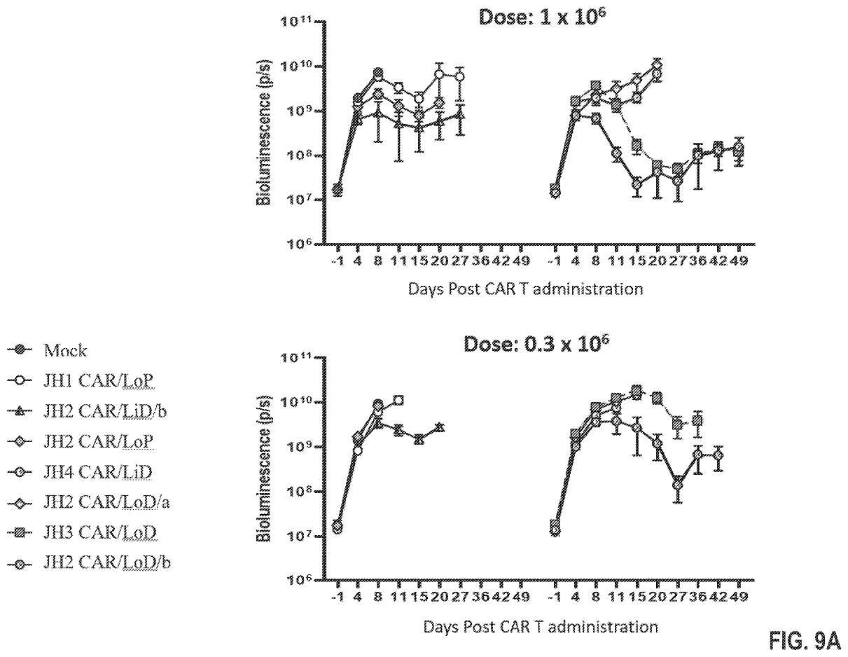

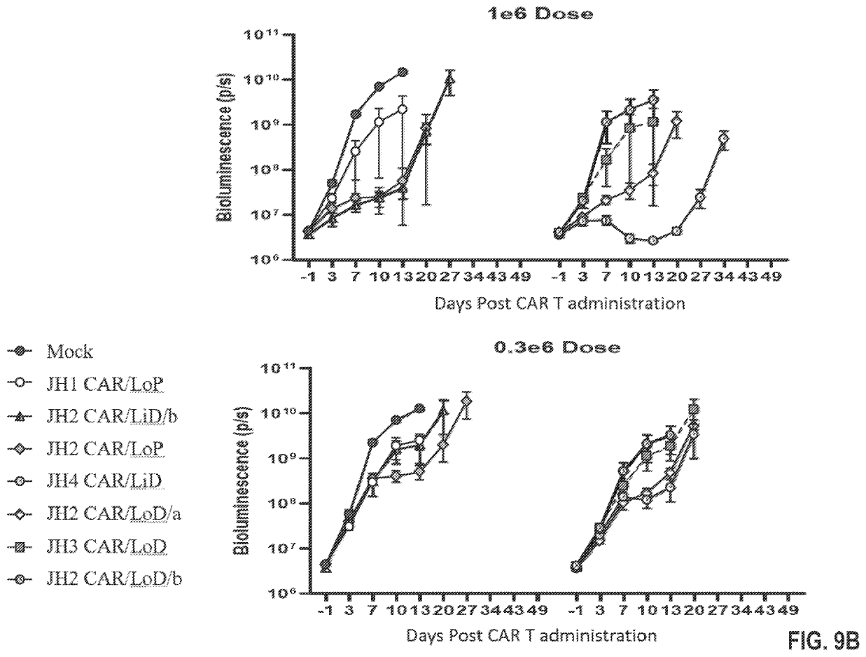

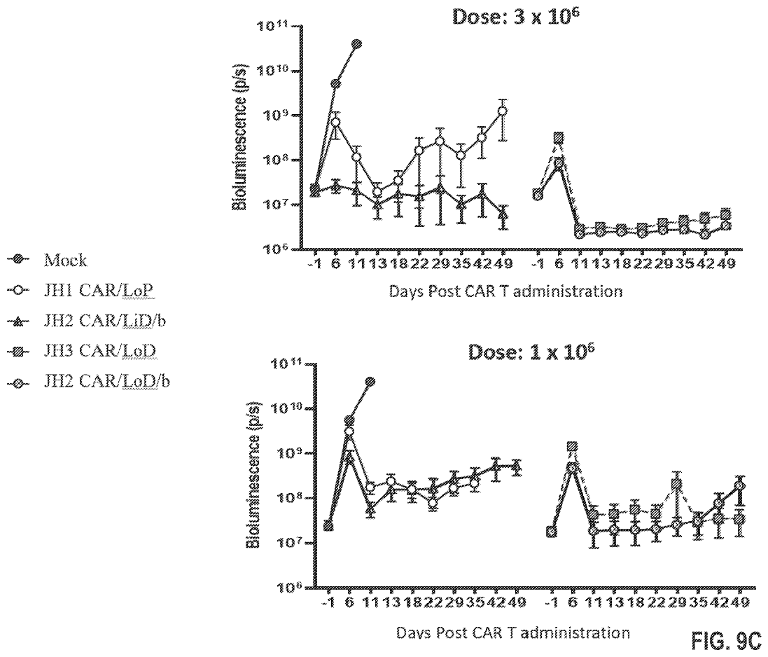

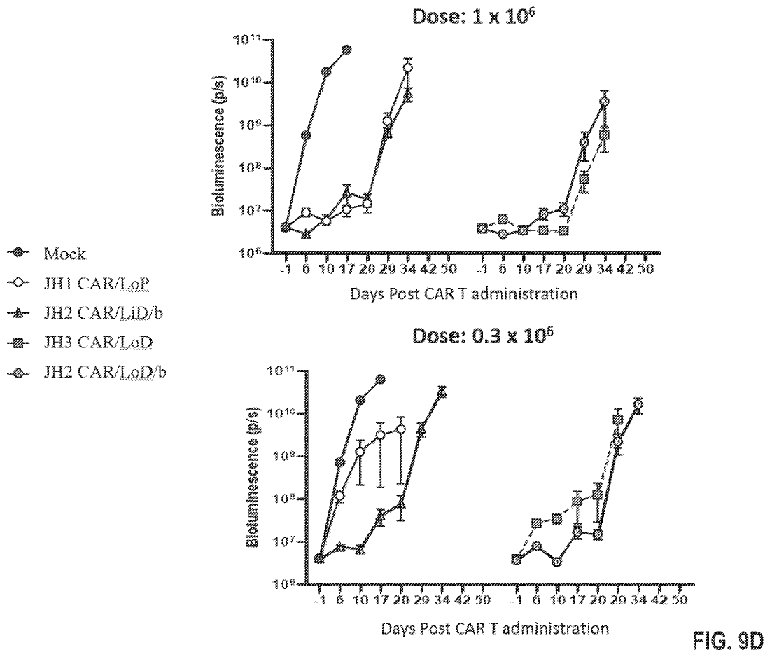

Across all doses and B cell tumor models the majority of the CD19×BAFF-R bispecific CAR-T cells exhibited superior tumor growth control relative to the Mock T cell controls. JH2 CAR/LoD/b and JH3 CAR/LOD exhibited sustained control of tumor growth relative to the other CAR candidates at both high and low doses in the Raji cell model (FIG. 9A) indicating that these two CARs were able to lessen tumor burden in mice after administration more effectively than other bispecific CAR-T cells in this model. In the Nalm6 model at a medium dose, JH4 CAR2/LiD yielded low relative bioluminescence up to 20 days post CAR T administration before increasing to levels similar to other CARs (FIG. 9B, top panel). At a low dose, this difference is less profound with tumor burden (bioluminescence) increasing in all mice over time (FIG. 9B, bottom panel).

Four CARs were then selected for further testing with a different T cell donor (Donor 20075). All four CD19x BAFF-R bispecific CAR-T cells exhibited superior tumor growth control relative to the Mock T cell controls. Administration of JH2 CAR/LoD/b and JH3 CAR/LoD yielded sustained low bioluminescence throughout the 50 day testing period in the Raji cell model at both the high and mid dose (FIG. 9C). Efficacy in the Nalm6 model was also observed with these second CAR-T donors, albeit with a relatively shorter duration compared to the Raji model (FIG. 9D).

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

SEQUENCE TABLE

| SEQ | | |
|---|---|---|
| ID | | |
| NO: | Sequence | Description |
| 1 | QVQLVQSGAEVKKPGASVKVSCKASGYTFTSYAMHWVRQAPGQRLEWMGWINAGN | JH1 V_{H} |
| | GNTKYSQKFQGRVTITRDTSASTAYMELRSLRSDDTAVYYCASGPGRLPVVWGQG | |
| | TLVTVSS | |
| 2 | SYVLTQPPSVSVAPGKTARITCGGNNIGSKSVHWYQQKPGQAPVLVVYDDSDRPS | JH1 V_{L} |
| | GIPERFSGSNSGNTATLTISRVEAGDEADYYCQVWDSSSDHVVFGGGTKLTVL | |
| 3 | QVQLQESGPGLVKPSETLSLTCTVSGGSISSYYWSWIRQPPGKGLEWIGYINNSG | JH2 V_{H} |
| | STNYNPSLKSRVTISVDTSKNQFSLKLSSVTAADTAVYYCAREDDYWGQGTLVTV | |
| | SS | |
| 4 | EIVMTQSPATLSLSPGERATLSCRASQIISSSYLSWYQQKPGQAPRLLIYGASTR | JH2 V_{L} |
| | ATGIPARFSGSGSGTDFTLTISSLQPEDFAVYYCQQDYDFPFTFGPGTKVDIKR | |
| 5 | QVQLQESGPGLVKPSETLSLTCTVSGGSISNYYWSWIRQPPGKGLEWIGYIYYSG | JH3 V_{H} |
| | STIYNPSLKSRVTISVDTSKNQFSLKLSSVTAADTAVYYCAGDSSGIDYWGQGTL | |
| | VTVSS | |
| 6 | EIVMTQSPATLSLSPGERATLSCRASQSVSSNYFSWYQQKPGQAPRLLIYGASTR | JH3 V_{L} |
| | ATGIPARFSGSGSGTDFTLTISSLQPEDFAVYYCQQDYDFPFTFGPGTKVDIK | |
| 7 | QVQLQESGPGLVKPSETLSLTCTVSGGSISSYYWSWIRQSPGKGLEWIGYIHYSG | JH4 V_{H} |
| | NTNYNPSLKSRVTISVDTSKNQFSLKLSSVTAADTAVYYCAREDDYWGQGTLVTV | |
| | SS | |
| 8 | EIVMTQSPATLSLSPGERATLSCRASQSVSSNYLSWYQQKPGQAPRLLIYGASTR | JH4 V_{L} |
| | ATGIPARFSGSGSGTDFTLTISSLQPEDFAVYYCQQDFDFPFTFGPGTKVDIK | |
| 9 | EVQLLESGGGVAQPGRSLRLSCAASGFTFSSYGMHWVRQAPGKGLEWVAVISYDG | JH5 V_{H} |
| | SNKYYADSVKGRFTISRDNSKNTLYLQMNSLRAEDTAVYYCAKDRLRGSYGMDVW | |
| | GQGTLVTVSS | |
| 10 | QSVLTQPPSVSGAPGQRVTISCTGSSSNIGAGYDVHWYQQLPGTAPKLLIYGNSN | JH5 V_{L} |
| | RPSGVPDRFSGSKSGTSASLAITGLQAEDEADYYCQSYDSSLSGSWVFGGGTKLT | |
| | VL | |
| 11 | QVQLVQSGAEVKKPGASVKVSCKASGYTFTSYAMHWVRQAPGQRLEWMGWINAGN | JH1 scFv |
| | GNTKYSQKFQGRVTITRDTSASTAYMELRSLRSDDTAVYYCASGPGRLPVVWGQG | |
| | TLVTVSSGGGGSGGGGSGGGGSSYVLTQPPSVSVAPGKTARITCGGNNIGSKSVH | |
| | WYQQKPGQAPVLVVYDDSDRPSGIPERFSGSNSGNTATLTISRVEAGDEADYYCQ | |
| | VWDSSSDHVVFGGGTKLTVL | |
| 12 | QVQLQESGPGLVKPSETLSLTCTVSGGSISSYYWSWIRQPPGKGLEWIGYINNSG | JH2 scFv |
| | STNYNPSLKSRVTISVDTSKNQFSLKLSSVTAADTAVYYCAREDDYWGQGTLVTV | |
| | SSGGGGSGGGGSGGGGSEIVMTQSPATLSLSPGERATLSCRASQIISSSYLSWYQ | |
| | QKPGQAPRLLIYGASTRATGIPARFSGSGSGTDFTLTISSLQPEDFAVYYCQQDY | |
| | DFPFTFGPGTKVDIK | |
| 13 | QVQLQESGPGLVKPSETLSLTCTVSGGSISNYYWSWIRQPPGKGLEWIGYIYYSG | JH3 scFv |
| | STIYNPSLKSRVTISVDTSKNQFSLKLSSVTAADTAVYYCAGDSSGIDYWGQGTL | |
| | VTVSSGGGGSGGGGSGGGGSEIVMTQSPATLSLSPGERATLSCRASQSVSSNYFS | |
| | WYQQKPGQAPRLLIYGASTRATGIPARFSGSGSGTDFTLTISSLQPEDFAVYYCQ | |
| | QDYDFPFTFGPGTKVDIK | |
| 14 | QVQLQESGPGLVKPSETLSLTCTVSGGSISSYYWSWIRQSPGKGLEWIGYIHYSG | JH4 scFv |
| | NTNYNPSLKSRVTISVDTSKNQFSLKLSSVTAADTAVYYCAREDDYWGQGTLVTV | |
| | SSGGGGSGGGGSGGGGSEIVMTQSPATLSLSPGERATLSCRASQSVSSNYLSWYQ | |
| | QKPGQAPRLLIYGASTRATGIPARFSGSGSGTDFTLTISSLQPEDFAVYYCQQDF | |
| | DFPFTFGPGTKVDIK | |
| 15 | EVQLLESGGGVAQPGRSLRLSCAASGFTFSSYGMHWVRQAPGKGLEWVAVISYDG | JH5 scFv |
| | SNKYYADSVKGRFTISRDNSKNTLYLQMNSLRAEDTAVYYCAKDRLRGSYGMDVW | |
| | GQGTLVTVSSGGGGSGGGGSGGGGSQSVLTQPPSVSGAPGQRVTISCTGSSSNIG | |
| | AGYDVHWYQQLPGTAPKLLIYGNSNRPSGVPDRFSGSKSGTSASLAITGLQAEDE | |
| | ADYYCQSYDSSLSGSWVFGGGTKLTVL | |
| 16 | GYTFTSY | JH1 CDR-H1 |
| 17 | NAGNGN | JH1 CDR-H2 |
| 18 | GPGRLPVV | JH1 CDR-H3 |
| 19 | GGNNIGSKSVH | JH1 CDR-L1 |
| 20 | DDSDRPS | JH1 CDR-L2 |
| 21 | QVWDSSSDHVV | JH1 CDR-L3 |
| 22 | GGSISSY | JH2 CDR-H1 |
| | | JH4 CDR-H1 |
| 23 | NNSGS | JH2 CDR-H2 |
| 24 | EDDY | JH2 CDR-H3 |
| | | JH4 CDR-H3 |
| 25 | RASQIISSSYLS | JH2 CDR-L1 |
| 26 | GASTRAT | JH2 CDR-L2 |
| | | JH3 CDR-L2 |
| | | JH4 CDR-L2 |
| 27 | QQDYDFPFT | JH2 CDR-L3 |
| | | JH3 CDR-L3 |
| 28 | GGSISNY | JH3 CDR-H1 |
| 29 | YYSGS | JH3 CDR-H2 |
| 30 | DSSGIDY | JH3 CDR-H3 |
| 31 | RASQSVSSNYFS | JH3 CDR-L1 |
| 32 | HYSGN | JH4 CDR-H2 |
| 33 | RASQSVSSNYLS | JH4 CDR-L1 |
| 34 | QQDFDFPFT | JH4 CDR-L3 |
| 35 | GFTFSSY | JH5 CDR-H1 |
| 36 | SYDGSN | JH5 CDR-H2 |
| 37 | DRLRGSYGMDV | JH5 CDR-H3 |
| 38 | TGSSSNIGAGYDVH | JH5 CDR-L1 |
| 39 | GNSNRPS | JH5 CDR-L2 |
| 40 | QSYDSSLSGSWV | JH5 CDR-L3 |
| 41 | EVKLQESGPGLVAPSQSLSVTCTVSGVSLPDYGVSWIRQPPRKGLEWLGVIWGSE | CD19 V_{H} |
| | TTYYNSALKSRLTIIKDNSKSQVFLKMNSLQTDDTAIYYCAKHYYYGGSYAMDYW | |
| | GQGTSVTVSS | |
| 42 | DIQMTQTTSSLSASLGDRVTISCRASQDISKYLNWYQQKPDGTVKLLIYHTSRLH | CD19 V_{L} |
| | SGVPSRFSGSGSGTDYSLTISNLEQEDIATYFCQQGNTLPYTFGGGTKLEIT | |
| 43 | DYGVS | CD19 CDR-H1 |
| 44 | VIWGSETTYYNSALKS | CD19 CDR-H2 |
| 45 | YAMDYWG | CD19 CDR-H3 |
| 46 | HYYYGGSYAMDY | CD19 CDR-H3 |
| 47 | RASQDISKYLN | CD19 CDR-L1 |
| 48 | SRLHSGV | CD19 CDR-L2 |
| 49 | HTSRLHS | CD19 CDR-L2 |
| 50 | GNTLPYTFG | CD19 CDR-L3 |
| 51 | QQGNTLPYT | CD19 CDR-L3 |
| 52 | ESKYGPPCPPCPM | Short Spacer |
| | | (IgG4 hinge) |
| | | (aa) |
| 53 | ESKYGPPCPPCPAPPVAGPSVFLFPPKPKDTLMISRTPEVTCVVVDVSQEDPEVQ | Long spacer |
| | FNWYVDGVEVHNAKTKPREEQFQSTYRVVSVLTVLHQDWLNGKEYKCKVSNKGLP | (IgG4/IgG2 |
| | SSIEKTISKAKGQPREPQVYTLPPSQEEMTKNQVSLTCLVKGFYPSDIAVEWESN | hinge-IgG2/ |
| | GQPENNYKTTPPVLDSDGSFFLYSRLTVDKSRWQEGNVFSCSVMHEALHNHYTQK | IgG4 CH2- |
| | SLSLSLGK | IgG4 CH3 |
| | | spacer |
| 54 | gaatctaagtacggaccgccctgccctccctgccctgctcctcctgtggctggac | IgG4/IgG2 |
| | caagcgtgttcctgtttccacctaagcctaaagataccctgatgatttcccgcac | hinge- |
| | acctgaagtgacttgcgtggtcgtggacgtgagccaggaggatccagaagtgcag | IgG2/IgG4 CH2- |
| | ttcaactggtacgtggacggcgtggaagtccacaatgctaagactaaaccccgag | IgG4 CH3 |
| | aggaacagtttcagtcaacttaccgggtcgtgagcgtgctgaccgtcctgcatca | spacer (nt) |
| | ggattggctgaacgggaaggagtataagtgcaaagtgtctaataagggactgcct | |
| | agctccatcgagaaaacaattagtaaggcaaaagggcagcctcgagaaccacagg | |
| | tgtataccctgccccctagccaggaggaaatgaccaagaaccaggtgtccctgac | |
| | atgtctggtcaaaggcttctatccaagtacatcgccgtggagtgggaatcaaatg | |
| | ggcagcccgagaacaattacaagaccacaccacccgtgctggactctgatggaag | |
| | tttctttctgtattccaggctgaccgtggataaatctcgctggcaggagggcaac | |
| | gtgttctcttgcagtgtcatgcacgaagccctgcacaatcattatacacagaagt | |
| | cactgagcctgtccctgggcaaa | |
| 55 | FWVLVVVGGVLACYSLLVTVAFIIFWV | CD28 |
| | | transmembrane |
| | | domain (aa) |
| 56 | KRGRKKLLYIFKQPFMRPVQTTQEEDGCSCRFPEEEEGGCEL | 4-1BB-derived |
| | | intracellular co- |
| | | signaling |
| | | sequence (aa) |
| 57 | RVKFSRSADAPAYQQGQNQLYNELNLGRREEYDVLDKRRGRDPEMGGKPRRKNPQ | CD3-zeta |
| | EGLYNELQKDKMAEAYSEIGMKGERRRGKGHDGLYQGLSTATKDTYDALHMQALP | derived |
| | PR | intracellular |
| | | signaling |
| | | domain (aa) |
| 58 | GGGGSGGGGSGGGGS | Linker |
| 59 | GSTSGSGKPGSGEGSTKG | Linker |
| 60 | GGGGS | Linker |
| 61 | GGGGSGGGGS | Linker |
| 62 | GGGGSGGGGSGGGGGGGGS | Linker |
| 63 | EVQLVESGGGLVQPGRSLRLSCAASGFTEDDYAMHWVRQAPGKGLEWVSGISWNS | CD19 clone 5 |
| | GRIGYADSVKGRFTISRDNAKNSLFLQMNSLRAEDTAVYYCARDQGYHYYDSAEH | V_{H}; |
| | AFDIWGQGTMVTVSS | CD19 clone 17 |
| | | V_{H} |
| 64 | EVQLVESGGGLVQPGRSLRLSCAASGFTEDDYAMHWVRQAPGKGLEWVSGISWNS | CD19 clone 18 |
| | GRIGYADSVKGRFTISRDNAKNSLFLQMNSLRAEDTAVYYCARDQGYHYYDSAEH | V_{H}; |
| | AFDIWGQGTVVTVSS | CD19 clone |
| | | 18B V_{H}; |
| | | CD19 clone 76 |
| | | V_{H} |
| 65 | SYELTQDPAVSVALGQTVRITCQGDSLRSYYASWYQQKPGQAPVLVIYDKNNRPS | CD19 clone 5 |
| | GIPDRFSGSSSGNTASLTITGAQAEDEADYYCNSRDSSGNNWVFGGGTKLTVL | V_{L} |
| 66 | QSALTQPASVSGSPGQSITIFCTGTSSDVGGYNYVSWYQQLPGTAPKLLIYSNNQ | CD19 clone 17 |
| | RPSGVPDRFSGSKSGTSASLAISGLRSEDEADYYCAAWDDSLSVVFGGGTKLTVL | V_{L} |
| 67 | QSALTQPRSVSGFPGQSVTISCTGTTSDDVSWYQQHPGKAPQLMLYDVSKRPSGV | CD19 clone 18 |
| | PHRFSGSRSGRAASLIISGLQTEDEADYFCCSYAGRYNSVLFGGGTKLTVL | V_{L} |
| 68 | QSALTQPRSVSGFPGQSVTISCTGTTSDDVSWYQQHPGKAPQLMLYDVSKRPSGV | CD19 clone |
| | PHRFSGSRSGRAASLIISGLQTEDEADYFCSSYAGRYNSVLFGGGTKLTVL | 18B V_{L} |
| 69 | QSVLTQPPSVSAAPGQEVTISCSGSSSNIGNNYVSWYQQLPGTAPKLLIYDNDKR | CD19 clone 76 |
| | PSGIPDRFSGSKSGTSATLGITGLQTGDEADYYCGTWDGNLSAVFGGGTKVTVL | V_{L} |
| 70 | EVQLVESGGGLVQPGRSLRLSCAASGFTEDDYAMHWVRQAPGKGLEWVSGISWNS | CD19 clone 5 |
| | GRIGYADSVKGRFTISRDNAKNSLFLQMNSLRAEDTAVYYCARDQGYHYYDSAEH | scFv |
| | AFDIWGQGTMVTVSSGGGGGGGGSGGGGSSYELTQDPAVSVALGQTVRITCQGD | |
| | SLRSYYASWYQQKPGQAPVLVIYDKNNRPSGIPDRFSGSSSGNTASLTITGAQAE | |
| | DEADYYCNSRDSSGNNWVFGGGTKLTVL | |
| 71 | EVQLVESGGGLVQPGRSLRLSCAASGFTEDDYAMHWVRQAPGKGLEWVSGISWNS | CD19 clone 17 |
| | GRIGYADSVKGRFTISRDNAKNSLFLQMNSLRAEDTAVYYCARDQGYHYYDSAEH | scFv |
| | AFDIWGQGTMVTVSSGGGGGGGGSGGGGSQSALTQPASVSGSPGQSITIFCTGT | |
| | SSDVGGYNYVSWYQQLPGTAPKLLIYSNNQRPSGVPDRFSGSKSGTSASLAISGL | |
| | RSEDEADYYCAAWDDSLSVVFGGGTKLTVL | |
| 72 | EVQLVESGGGLVQPGRSLRLSCAASGFTEDDYAMHWVRQAPGKGLEWVSGISWNS | CD19 clone 18 |
| | GRIGYADSVKGRFTISRDNAKNSLFLQMNSLRAEDTAVYYCARDQGYHYYDSAEH | scFv |
| | AFDIWGQGTVVTVSSGGGGGGGGSGGGGSQSALTQPRSVSGFPGQSVTISCTGT | |
| | TSDDVSWYQQHPGKAPQLMLYDVSKRPSGVPHRFSGSRSGRAASLIISGLQTEDE | |
| | ADYFCCSYAGRYNSVLFGGGTKLTVL | |
| 73 | EVQLVESGGGLVQPGRSLRLSCAASGFTEDDYAMHWVRQAPGKGLEWVSGISWNS | CD19 clone |
| | GRIGYADSVKGRFTISRDNAKNSLFLQMNSLRAEDTAVYYCARDQGYHYYDSAEH | 18B scFv |
| | AFDIWGQGTVVTVSSGGGGSGGGGSGGGGSQSALTQPRSVSGFPGQSVTISCTGT | |
| | TSDDVSWYQQHPGKAPQLMLYDVSKRPSGVPHRFSGSRSGRAASLIISGLQTEDE | |
| | ADYFCSSYAGRYNSVLFGGGTKLTVL | |
| 74 | EVQLVESGGGLVQPGRSLRLSCAASGFTEDDYAMHWVRQAPGKGLEWVSGISWNS | CD19 clone 76 |
| | GRIGYADSVKGRFTISRDNAKNSLFLQMNSLRAEDTAVYYCARDQGYHYYDSAEH | scFv |
| | AFDIWGQGTVVTVSSGGGGGGGGSGGGGSQSVLTQPPSVSAAPGQEVTISCSGS | |
| | SSNIGNNYVSWYQQLPGTAPKLLIYDNDKRPSGIPDRFSGSKSGTSATLGITGLQ | |
| | TGDEADYYCGTWDGNLSAVFGGGTKVTVL | |
| 75 | DYAMH | CD19 clone 5, |
| | | 17, 18, 18B, 76 |
| | | V_{H} CDR1 |
| 76 | GISWNSGRIGYADSVKG | CD19 clone 5, |
| | | 17, 18, 18B, 76 |
| | | V_{H} CDR2 |
| 77 | DQGYHYYDSAEHAFDI | CD19 clone 5, |
| | | 17, 18, 18B, 76 |
| | | V_{H} CDR3 |
| 78 | QGDSLRSYYAS | CD19 clone 5 |
| | | V_{L} CDR1 |
| 79 | DKNNRPS | CD19 clone 5 |
| | | V_{L} CDR2 |
| 80 | NSRDSSGNNWV | CD19 clone 5 |
| | | V_{L} CDR3 |
| 81 | TGTSSDVGGYNYVS | CD19 clone 17 |
| | | V_{L} CDR1 |
| 82 | SNNQRPS | CD19 clone 17 |
| | | V_{L} CDR2 |
| 83 | AAWDDSLSVV | CD19 clone 17 |
| | | V_{L} CDR3 |
| 84 | TGTTSDDVS | CD19 clone 18 |
| | | V_{L} CDR1; |
| | | CD19 clone |
| | | 18B V_{L} CDR1 |
| 85 | DVSKRPS | CD19 clone 18 |
| | | V_{L} CDR2; |
| | | CD19 clone |
| | | 18B V_{L} CDR2 |
| 86 | CSYAGRYNSVL | CD19 clone 18 |
| | | V_{L} CDR3 |
| 87 | SSYAGRYNSVL | CD19 clone |
| | | 18B V_{L} CDR3 |
| 88 | SGSSSNIGNNYVS | CD19 clone 76 |
| | | V_{L} CDR1 |
| 89 | DNDKRPS | CD19 clone 76 |
| | | V_{L} CDR2 |
| 90 | GTWDGNLSAV | CD19 clone 76 |
| | | V_{L} CDR3 |
| 91 | X1PPX2P,X1 is glycine,cysteine rarginine and X2 is | Hinge |
| | cysteine or threonine | consensus |
| 92 | ASTKGPSVFPLAPCSRSTSESTAALGCLVKDYFPEPVTVSWNSGALTSGV | Human IgG2 Fc |
| | HTFPAVLQSSGLYSLSSVVTVPSSNFGTQTYTCNVDHKPSNTKVDKTVER | (Uniprot |
| | KCCVECPPCPAPPVAGPSVFLFPPKPKDTLMISRTPEVTCVVVDVSHEDP | P01859) |
| | EVQFNWYVDGVEVHNAKTKPREEQFNSTFRVVSVLTVVHQDWLNGKEYKC | |
| | KVSNKGLPAPIEKTISKTKGQPREPQVYTLPPSREEMTKNQVSLTCLVKG | |
| | FYPSDISVEWESNGQPENNYKTTPPMLDSDGSFFLYSKLTVDKSRWQQGN | |
| | VFSCSVMHEALHNHYTQKSLSLSPGK | |
| 93 | ASTKGPSVFPLAPCSRSTSESTAALGCLVKDYFPEPVTVSWNSGALTSGV | Human IgG4 Fc |
| | HTFPAVLQSSGLYSLSSVVTVPSSSLGTKTYTCNVDHKPSNTKVDKRVES | (Uniprot |
| | KYGPPCPSCPAPEFLGGPSVFLFPPKPKDTLMISRTPEVTCVVVDVSQED | P01861) |
| | PEVQFNWYVDGVEVHNAKTKPREEQFNSTYRVVSVLTVLHQDWLNGKEYK | |
| | CKVSNKGLPSSIEKTISKAKGQPREPQVYTLPPSQEEMTKNQVSLTCLVK | |
| | GFYPSDIAVEWESNGQPENNYKTTPPVLDSDGSFFLYSRLTVDKSRWQEG | |
| | NVFSCSVMHEALHNHYTQKSLSLSLGK | |
| 94 | DIQMTQTTSSLSASLGDRVTISCRASQDISKYLNWYQQKPDGTVKLLIYH | JH1 CAR/LOP |
| | TSRLHSGVPSRFSGSGSGTDYSLTISNLEQEDIATYFCQQGNTLPYTFGG | |
| | GTKLEITGGGGSQVQLVQSGAEVKKPGASVKVSCKASGYTFTSYAMHWVR | |
| | QAPGQRLEWMGWINAGNGNTKYSQKFQGRVTITRDTSASTAYMELRSLRS | |
| | DDTAVYYCASGPGRLPVVWGQGTLVTVSSGGGGSGGGGSGGGGSSYVLTQ | |
| | PPSVSVAPGKTARITCGGNNIGSKSVHWYQQKPGQAPVLVVYDDSDRPSG | |
| | IPERFSGSNSGNTATLTISRVEAGDEADYYCQVWDSSSDHVVFGGGTKLT | |
| | VLGGGGSEVKLQESGPGLVAPSQSLSVTCTVSGVSLPDYGVSWIRQPPRK | |
| | GLEWLGVIWGSETTYYNSALKSRLTIIKDNSKSQVFLKMNSLQTDDTAIY | |
| | YCAKHYYYGGSYAMDYWGQGTSVTVSSESKYGPPCPPCPMFWVLVVVGGV | |
| | LACYSLLVTVAFIIFWVKRGRKKLLYIFKQPFMRPVQTTQEEDGCSCRFP | |
| | EEEEGGCELRVKFSRSADAPAYQQGQNQLYNELNLGRREEYDVLDKRRGR | |
| | DPEMGGKPRRKNPQEGLYNELQKDKMAEAYSEIGMKGERRRGKGHDGLYQ | |
| | GLSTATKDTYDALHMQALPPR | |
| 95 | DIQMTQTTSSLSASLGDRVTISCRASQDISKYLNWYQQKPDGTVKLLIYH | JH2 CAR/LiD/a |
| | TSRLHSGVPSRFSGSGSGTDYSLTISNLEQEDIATYFCQQGNTLPYTFGG | |
| | GTKLEITGSTSGSGKPGSGEGSTKGEVKLQESGPGLVAPSQSLSVTCTVS | |
| | GVSLPDYGVSWIRQPPRKGLEWLGVIWGSETTYYNSALKSRLTIIKDNSK | |
| | SQVFLKMNSLQTDDTAIYYCAKHYYYGGSYAMDYWGQGTSVTVSSGGGGS | |
| | QVQLQESGPGLVKPSETLSLTCTVSGGSISSYYWSWIRQPPGKGLEWIGY | |
| | INNSGSTNYNPSLKSRVTISVDTSKNQFSLKLSSVTAADTAVYYCAREDD | |
| | YWGQGTLVTVSSGGGGSGGGGSGGGGSEIVMTQSPATLSLSPGERATLSC | |
| | RASQIISSSYLSWYQQKPGQAPRLLIYGASTRATGIPARFSGSGSGTDFT | |
| | LTISSLQPEDFAVYYCQQDYDFPFTFGPGTKVDIKRESKYGPPCPPCPMF | |
| | WVLVVVGGVLACYSLLVTVAFIIFWVKRGRKKLLYIFKQPFMRPVQTTQE | |
| | EDGCSCRFPEEEEGGCELRVKFSRSADAPAYQQGQNQLYNELNLGRREEY | |
| | DVLDKRRGRDPEMGGKPRRKNPQEGLYNELQKDKMAEAYSEIGMKGERRR | |
| | GKGHDGLYQGLSTATKDTYDALHMQALPPR | |
| 96 | DIQMTQTTSSLSASLGDRVTISCRASQDISKYLNWYQQKPDGTVKLLIYH | JH2 CAR/LOP |
| | TSRLHSGVPSRFSGSGSGTDYSLTISNLEQEDIATYFCQQGNTLPYTFGG | |
| | GTKLEITGGGGSQVQLQESGPGLVKPSETLSLTCTVSGGSISSYYWSWIR | |
| | QPPGKGLEWIGYINNSGSTNYNPSLKSRVTISVDTSKNQFSLKLSSVTAA | |
| | DTAVYYCAREDDYWGQGTLVTVSSGGGGSGGGGSGGGGSEIVMTQSPATL | |
| | SLSPGERATLSCRASQIISSSYLSWYQQKPGQAPRLLIYGASTRATGIPA | |
| | RFSGSGSGTDFTLTISSLQPEDFAVYYCQQDYDFPFTFGPGTKVDIKRGG | |
| | GGSEVKLQESGPGLVAPSQSLSVTCTVSGVSLPDYGVSWIRQPPRKGLEW | |
| | LGVIWGSETTYYNSALKSRLTIIKDNSKSQVFLKMNSLQTDDTAIYYCAK | |
| | HYYYGGSYAMDYWGQGTSVTVSSESKYGPPCPPCPMFWVLVVVGGVLACY | |
| | SLLVTVAFIIFWVKRGRKKLLYIFKQPFMRPVQTTQEEDGCSCRFPEEEE | |
| | GGCELRVKFSRSADAPAYQQGQNQLYNELNLGRREEYDVLDKRRGRDPEM | |
| | GGKPRRKNPQEGLYNELQKDKMAEAYSEIGMKGERRRGKGHDGLYQGLST | |
| | ATKDTYDALHMQALPPR | |
| 97 | DIQMTQTTSSLSASLGDRVTISCRASQDISKYLNWYQQKPDGTVKLLIYH | JH2 CAR/LiD/b |
| | TSRLHSGVPSRFSGSGSGTDYSLTISNLEQEDIATYFCQQGNTLPYTFGG | |
| | GTKLEITGSTSGSGKPGSGEGSTKGEVKLQESGPGLVAPSQSLSVTCTVS | |
| | GVSLPDYGVSWIRQPPRKGLEWLGVIWGSETTYYNSALKSRLTIIKDNSK | |
| | SQVFLKMNSLQTDDTAIYYCAKHYYYGGSYAMDYWGQGTSVTVSSGGGGS | |
| | EIVMTQSPATLSLSPGERATLSCRASQIISSSYLSWYQQKPGQAPRLLIY | |
| | GASTRATGIPARFSGSGSGTDFTLTISSLQPEDFAVYYCQQDYDFPFTFG | |
| | PGTKVDIKRGGGGSGGGGSGGGGSQVQLQESGPGLVKPSETLSLTCTVSG | |
| | GSISSYYWSWIRQPPGKGLEWIGYINNSGSTNYNPSLKSRVTISVDTSKN | |
| | QFSLKLSSVTAADTAVYYCAREDDYWGQGTLVTVSSESKYGPPCPPCPMF | |
| | WVLVVVGGVLACYSLLVTVAFIIFWVKRGRKKLLYIFKQPFMRPVQTTQE | |
| | EDGCSCRFPEEEEGGCELRVKFSRSADAPAYQQGQNQLYNELNLGRREEY | |
| | DVLDKRRGRDPEMGGKPRRKNPQEGLYNELQKDKMAEAYSEIGMKGERRR | |
| | GKGHDGLYQGLSTATKDTYDALHMQALPPR | |
| 98 | EIVMTQSPATLSLSPGERATLSCRASQIISSSYLSWYQQKPGQAPRLLIY | JH2 |
| | GASTRATGIPARFSGSGSGTDFTLTISSLQPEDFAVYYCQQDYDFPFTFG | CAR/LoD/a |
| | PGTKVDIKRGGGGSGGGGSDIQMTQTTSSLSASLGDRVTISCRASQDISK | |
| | YLNWYQQKPDGTVKLLIYHTSRLHSGVPSRFSGSGSGTDYSLTISNLEQE | |
| | DIATYFCQQGNTLPYTFGGGTKLEITGSTSGSGKPGSGEGSTKGEVKLQE | |
| | SGPGLVAPSQSLSVTCTVSGVSLPDYGVSWIRQPPRKGLEWLGVIWGSET | |
| | TYYNSALKSRLTIIKDNSKSQVFLKMNSLQTDDTAIYYCAKHYYYGGSYA | |
| | MDYWGQGTSVTVSS | |
| | GGGGSGGGGSQVQLQESGPGLVKPSETLSLTCTVSGGSISSYYWSWIRQP | |
| | PGKGLEWIGYINNSGSTNYNPSLKSRVTISVDTSKNQFSLKLSSVTAADT | |
| | AVYYCAREDDYWGQGTLVTVSSESKYGPPCPPCPMFWVLVVVGGVLACYS | |
| | LLVTVAFIIFWVKRGRKKLLYIFKQPFMRPVQTTQEEDGCSCRFPEEEEG | |
| | GCELRVKFSRSADAPAYQQGQNQLYNELNLGRREEYDVLDKRRGRDPEMG | |
| | GKPRRKNPQEGLYNELQKDKMAEAYSEIGMKGERRRGKGHDGLYQGLSTA | |
| | TKDTYDALHMQALPPR | |
| 99 | QVQLQESGPGLVKPSETLSLTCTVSGGSISSYYWSWIRQPPGKGLEWIGY | JH2 |
| | INNSGSTNYNPSLKSRVTISVDTSKNQFSLKLSSVTAADTAVYYCAREDD | CAR/LoD/b |
| | YWGQGTLVTVSSGGGGSDIQMTQTTSSLSASLGDRVTISCRASQDISKYL | |
| | NWYQQKPDGTVKLLIYHTSRLHSGVPSRFSGSGSGTDYSLTISNLEQEDI | |
| | ATYFCQQGNTLPYTFGGGTKLEITGSTSGSGKPGSGEGSTKGEVKLQESG | |
| | PGLVAPSQSLSVTCTVSGVSLPDYGVSWIRQPPRKGLEWLGVIWGSETTY | |
| | YNSALKSRLTIIKDNSKSQVFLKMNSLQTDDTAIYYCAKHYYYGGSYAMD | |
| | YWGQGTSVTVSSGGGGSEIVMTQSPATLSLSPGERATLSCRASQIISSSY | |
| | LSWYQQKPGQAPRLLIYGASTRATGIPARFSGSGSGTDFTLTISSLQPED | |
| | FAVYYCQQDYDFPFTFGPGTKVDIKRESKYGPPCPPCPMFWVLVVVGGVL | |
| | ACYSLLVTVAFIIFWVKRGRKKLLYIFKQPFMRPVQTTQEEDGCSCRFPE | |
| | EEEGGCELRVKFSRSADAPAYQQGQNQLYNELNLGRREEYDVLDKRRGRD | |
| | PEMGGKPRRKNPQEGLYNELQKDKMAEAYSEIGMKGERRRGKGHDGLYQG | |
| | LSTATKDTYDALHMQALPPR | |
| 100 | EIVMTQSPATLSLSPGERATLSCRASQSVSSNYFSWYQQKPGQAPRLLIY | JH3 CAR/LOD |
| | GASTRATGIPARFSGSGSGTDFTLTISSLQPEDFAVYYCQQDYDFPFTFG | |
| | PGTKVDIKRGGGGSGGGGSDIQMTQTTSSLSASLGDRVTISCRASQDISK | |
| | YLNWYQQKPDGTVKLLIYHTSRLHSGVPSRFSGSGSGTDYSLTISNLEQE | |
| | DIATYFCQQGNTLPYTFGGGTKLEITGSTSGSGKPGSGEGSTKGEVKLQE | |
| | SGPGLVAPSQSLSVTCTVSGVSLPDYGVSWIRQPPRKGLEWLGVIWGSET | |
| | TYYNSALKSRLTIIKDNSKSQVFLKMNSLQTDDTAIYYCAKHYYYGGSYA | |
| | MDYWGQGTSVTVSS | |
| | GGGGSGGGGSQVQLQESGPGLVKPSETLSLTCTVSGGSISNYYWSWIRQP | |
| | PGKGLEWIGYIYYSGSTIYNPSLKSRVTISVDTSKNQFSLKLSSVTAADT | |
| | AVYYCAGDSSGIDYWGQGTLVTVSSESKYGPPCPPCPMFWVLVVVGGVLA | |
| | CYSLLVTVAFIIFWVKRGRKKLLYIFKQPFMRPVQTTQEEDGCSCRFPEE | |
| | EEGGCELRVKFSRSADAPAYQQGQNQLYNELNLGRREEYDVLDKRRGRDP | |
| | EMGGKPRRKNPQEGLYNELQKDKMAEAYSEIGMKGERRRGKGHDGLYQGL | |
| | STATKDTYDALHMQALPPR | |
| 101 | DIQMTQTTSSLSASLGDRVTISCRASQDISKYLNWYQQKPDGTVKLLIYH | JH4 CAR/LiD |
| | TSRLHSGVPSRFSGSGSGTDYSLTISNLEQEDIATYFCQQGNTLPYTFGG | |
| | GTKLEITGSTSGSGKPGSGEGSTKGEVKLQESGPGLVAPSQSLSVTCTVS | |
| | GVSLPDYGVSWIRQPPRKGLEWLGVIWGSETTYYNSALKSRLTIIKDNSK | |
| | SQVFLKMNSLQTDDTAIYYCAKHYYYGGSYAMDYWGQGTSVTVSSGGGGS | |
| | QVQLQESGPGLVKPSETLSLTCTVSGGSISSYYWSWIRQSPGKGLEWIGY | |
| | IHYSGNTNYNPSLKSRVTISVDTSKNQFSLKLSSVTAADTAVYYCAREDD | |
| | YWGQGTLVTVSSGGGGSGGGGSGGGGSEIVMTQSPATLSLSPGERATLSC | |
| 102 | RASQSVSSNYLSWYQQKPGQAPRLLIYGASTRATGIPARFSGSGSGTDFT | |
| | LTISSLQPEDFAVYYCQQDFDFPFTFGPGTKVDIKRESKYGPPCPPCPMF | |
| | WVLVVVGGVLACYSLLVTVAFIIFWVKRGRKKLLYIFKQPFMRPVQTTQE | |
| | EDGCSCRFPEEEEGGCELRVKFSRSADAPAYQQGQNQLYNELNLGRREEY | |
| | DVLDKRRGRDPEMGGKPRRKNPQEGLYNELQKDKMAEAYSEIGMKGERRR | |
| | GKGHDGLYQGLSTATKDTYDALHMQALPPR | |
| 103 | DIQMTQTTSSLSASLGDRVTISCRASQDISKYLNWYQQKPDGTVKLLIYH | JH2 CAR/LiD/c |
| | TSRLHSGVPSRFSGSGSGTDYSLTISNLEQEDIATYFCQQGNTLPYTFGG | JH5 CAR/LOP |
| | GTKLEITGSTSGSGKPGSGEGSTKGEVKLQESGPGLVAPSQSLSVTCTVS | |
| | GVSLPDYGVSWIRQPPRKGLEWLGVIWGSETTYYNSALKSRLTIIKDNSK | |
| | SQVFLKMNSLQTDDTAIYYCAKHYYYGGSYAMDYWGQGTSVTVSSGGGGS | |
| | GGGGSGGGGSGGGGSEIVMTQSPATLSLSPGERATLSCRASQIISSSYLS | |
| | WYQQKPGQAPRLLIYGASTRATGIPARFSGSGSGTDFTLTISSLQPEDFA | |
| | VYYCQQDYDFPFTFGPGTKVDIKRGGGGSGGGGSGGGGSGGGGSQVQLQE | |
| | SGPGLVKPSETLSLTCTVSGGSISSYYWSWIRQPPGKGLEWIGYINNSGS | |
| | TNYNPSLKSRVTISVDTSKNQFSLKLSSVTAADTAVYYCAREDDYWGQGT | |
| | LVTVSSESKYGPPCPPCPMFWVLVVVGGVLACYSLLVTVAFIIFWVKRGR | |
| | KKLLYIFKQPFMRPVQTTQEEDGCSCRFPEEEEGGCELRVKFSRSADAPA | |
| | YQQGQNQLYNELNLGRREEYDVLDKRRGRDPEMGGKPRRKNPQEGLYNEL | |
| | QKDKMAEAYSEIGMKGERRRGKGHDGLYQGLSTATKDTYDALHMQALPPR | |
| | DIQMTQTTSSLSASLGDRVTISCRASQDISKYLNWYQQKPDGTVKLLIYH | |
| | TSRLHSGVPSRFSGSGSGTDYSLTISNLEQEDIATYFCQQGNTLPYTFGG | |
| | GTKLEITGGGGSEVQLLESGGGVAQPGRSLRLSCAASGFTFSSYGMHWVR | |
| | QAPGKGLEWVAVISYDGSNKYYADSVKGRFTISRDNSKNTLYLQMNSLRA | |
| | EDTAVYYCAKDRLRGSYGMDVWGQGTLVTVSSGGGGSGGGGSGGGGSQSV | |
| | LTQPPSVSGAPGQRVTISCTGSSSNIGAGYDVHWYQQLPGTAPKLLIYGN | |
| | SNRPSGVPDRFSGSKSGTSASLAITGLQAEDEADYYCQSYDSSLSGSWVF | |
| | GGGTKLTVLGGGGS | |
| | EVKLQESGPGLVAPSQSLSVTCTVSGVSLPDYGVSWIRQPPRKGLEWLGV | |
| | IWGSETTYYNSALKSRLTIIKDNSKSQVFLKMNSLQTDDTAIYYCAKHYY | |
| | YGGSYAMDYWGQGTSVTVSSESKYGPPCPPCPMFWVLVVVGGVLACYSLL | |
| | VTVAFIIFWVKRGRKKLLYIFKQPFMRPVQTTQEEDGCSCRFPEEEEGGC | |
| | ELRVKFSRSADAPAYQQGQNQLYNELNLGRREEYDVLDKRRGRDPEMGGK | |
| | PRRKNPQEGLYNELQKDKMAEAYSEIGMKGERRRGKGHDGLYQGLSTATK | |
| | DTYDALHMQALPPR | |
| 104 | MPLLLLLPLLWAGALA | CD33 signal |
| | | peptide |
| 105 | ggatctgcgatcgctccggtgcccgtcagtgggcagagcgcacatcgcccacagt | EF1 alpha |
| | ccccgagaagttggggggaggggtcggcaattgaaccggtgcctagagaaggtgg | promoter with |
| | cgcggggtaaactgggaaagtgatgtcgtgtactggctccgcctttttcccgagg | HTLV1 |
| | gtgggggagaaccgtatataagtgcagtagtcgccgtgaacgttctttttcgcaa | enhancer |
| | cgggtttgccgccagaacacagctgaagcttcgaggggctcgcatctctccttca | |
| | cgcgcccgccgccctacctgaggccgccatccacgccggttgagtcgcgttctgc | |
| | cgcctcccgcctgtggtgcctcctgaactgcgtccgccgtctaggtaagtttaaa | |
| | gctcaggtcgagaccgggcctttgtccggcgctcccttggagcctacctagactc | |
| | agccggctctccacgctttgcctgaccctgcttgctcaactctacgtctttgttt | |
| | cgttttctgttctgcgccgttacagatccaagctgtgaccggcgcctac | |
| 106 | aatcaacctctggattacaaaatttgtgaaagattgactggtattcttaactatg | Woodchuck |
| | ttgctccttttacgctatgtggatacgctgctttaatgcctttgtatcatgctat | Hepatitis Virus |
| | tgcttcccgtatggctttcattttctcctccttgtataaatcctggttgctgtct | (WHP) |
| | ctttatgaggagttgtggcccgttgtcaggcaacgtggcgtggtgtgcactgtgt | Post- |
| | ttgctgacgcaacccccactggttggggcattgccaccacctgtcagctcctttc | transcriptional |
| | cgggactttcgctttccccctccctattgccacggcggaactcatcgccgcctgc | Regulatory |
| | cttgcccgctgctggacaggggctcggctgttgggcactgacaattccgtggtgt | Element |
| | tgtcggggaaatcatcgtcctttccttggctgctcgcctgtgttgccacctggat | (WPRE) |
| | tctgcgcgggacgtccttctgctacgtcccttcggccctcaatccagcggacctt | |
| | ccttcccgcggcctgctgccggctctgcggcctcttccgcgtcttcgccttcgcc | |
| | ctcagacgagtcggatctccctttgggccgcctccccgc | |
| 107 | VKQTLNFDLLKLAGDVESNPGP | F2A peptide |
| 108 | GSGVKQTLNFDLLKLAGDVESNPGP | F2A peptide |
| 109 | QCTNYALLKLAGDVESNPGP | E2A peptide |
| 110 | GSGQCTNYALLKLAGDVESNPGP | E2A peptide |
| 111 | LEGGGEGRGSLLTCGDVEENPGPR | T2A peptide |
| 112 | EGRGSLLTCGDVEENPGP | T2A peptide |
| 113 | GSGEGRGSLLTCGDVEENPGP | T2A peptide |
| 114 | GSGATNFSLLKQAGDVEENPGP | P2A peptide |
| 115 | ATNFSLLKQAGDVEENPGP | P2A peptide |
| 116 | MLLLVTSLLLCELPHPAFLLIPRKVCNGIGIGEFKDSLSINATNIKHFKNCTSIS | tEGFR |
| | GDLHILPVAFRGDSFTHTPPLDPQELDILKTVKEITGFLLIQAWPENRTDLHAFE | |
| | NLEIIRGRTKQHGQFSLAVVSLNITSLGLRSLKEISDGDVIISGNKNLCYANTIN | |
| | WKKLFGTSGQKTKIISNRGENSCKATGQVCHALCSPEGCWGPEPRDCVSCRNVSR | |
| | GRECVDKCNLLEGEPREFVENSECIQCHPECLPQAMNITCTGRGPDNCIQCAHYI | |
| | DGPHCVKTCPAGVMGENNTLVWKYADAGHVCHLCHPNCTYGCTGPGLEGCPTNGP | |
| | KIPSIATGMVGALLLLLVVALGIGLEM | |
| 117 | RKVCNGIGIGEFKDSLSINATNIKHFKNCTSISGDLHILPVAFRGDSFTHTPPLD | tEGFR |
| | PQELDILKTVKEITGFLLIQAWPENRTDLHAFENLEIIRGRTKQHGQFSLAVVSL | |
| | NITSLGLRSLKEISDGDVIISGNKNLCYANTINWKKLFGTSGQKTKIISNRGENS | |
| | CKATGQVCHALCSPEGCWGPEPRDCVSCRNVSRGRECVDKCNLLEGEPREFVENS | |
| | ECIQCHPECLPQAMNITCTGRGPDNCIQCAHYIDGPHCVKTCPAGVMGENNTLVW | |
| | KYADAGHVCHLCHPNCTYGCTGPGLEGCPTNGPKIPSIATGMVGALLLLLVVALG | |
| | IGLFM | |
| 118 | MLLLVTSLLLCELPHPAFLLIPRKVCNGIGIGEFKDSLSINATNIKHFKNCTSIS | Truncated |
| | GDLHILPVAFRGDSFTHTPPLDPQELDILKTVKEITGELLIQAWPENRTDLHAFE | EGFR (tEGFR) |
| | NLEIIRGRTKQHGQFSLAVVSLNITSLGLRSLKEISDGDVIISGNKNLCYANTIN | |
| | WKKLFGTSGQKTKIISNRGENSCKATGQVCHALCSPEGCWGPEPRDCVSCRNVSR | |
| | GRECVDKCNLLEGEPREFVENSECIQCHPECLPQAMNITCTGRGPDNCIQCAHYI | |
| | DGPHCVKTCPAGVMGENNTLVWKYADAGHVCHLCHPNCTYGCTGPGLEGCPTNGP | |
| | KIPSIATGMVGALLLLLVVALGIGLFM | |
| 119 | DIQMTQTTSSLSASLGDRVTISCRASQDISKYLNWYQQKPDGTVKLLIYHTSRLH | CD19 scFv |
| | SGVPSRFSGSGSGTDYSLTISNLEQEDIATYFCQQGNTLPYTFGGGTKLEITGST | |
| | SGSGKPGSGEGSTKGEVKLQESGPGLVAPSQSLSVTCTVSGVSLPDYGVSWIRQP | |
| | PRKGLEWLGVIWGSETTYYNSALKSRLTIIKDNSKSQVFLKMNSLQTDDTAIYYC | |
| | AKHYYYGGSYAMDYWGQGTSVTVSS | |
| 120 | MRRGPRSLRGRDAPAPTPCVPAECFDLLVRHCVACGLLRTPRPKPAGASSPAPRT | Human BAFF-R |
| | ALQPQESVGAGAGEAALPLPGLLFGAPALLGLALVLALVLVGLVSWRRRQRRLRG | |
| | ASSAEAPDGDKDAPEPLDKVIILSPGISDATAPAWPPPGEDPGTTPPGHSVPVPA | |
| | TELGSTELVTTKTAGPEQQ | |
| 121 | KASQNVGTNVA | CDR L1 |
| 122 | SATYRNS | CDR L2 |
| 123 | QQYNRYPYT | CDR L3 |
| 124 | SYWMN | CDR H1 |
| 125 | QIYPGDGDTNYNGKFKG | CDR H2 |
| 126 | KTISSVVDFYFDY | CDR H3 |
| 127 | EVKLQQSGAELVRPGSSVKISCKASGYAFSSYWMNWVKQRPGQGLEWIGQIYPGD | V_{H} |
| | GDTNYNGKFKGQATLTADKSSSTAYMQLSGLTSEDSAVYFCARKTISSVVDFYFD | |
| | YWGQGTTVTVSS | |
| 128 | DIELTQSPKFMSTSVGDRVSVTCKASQNVGINVAWYQQKPGQSPKPLIYSATYRN | V_{L} |
| | SGVPDRFTGSGSGTDFTLTITNVQSKDLADYFCQQYNRYPYTSGGGTKLEIKR | |
| 129 | CTCTATCAATGAGAGAGCAATCTCCTGGTAATGTGATAGATTTCCCAACTTAATG | JH2 |
| | CCAACATACCATAAACCTCCCATTCTGCTAATGCCCAGCCTAAGTTGGGGAGACC | CAR/LoD/b |
| | ACTCCAGATTCCAAGATGTACAGTTTGCTTTGCTGGGCCTTTTTCCCATGCCTGC | (nucleic acid) |
| | CTTTACTCTGCCAGAGTTATATTGCTGGGGTTTTGAAGAAGATCCTATTAAATAA | |
| | AAGAATAAGCAGTATTATTAAGTAGCCCTGCATTTCAGGTTTCCTTGAGTGGCAG | |
| | GCCAGGCCTGGCCGTGAACGTTCACTGAAATCATGGCCTCTTGGCCAAGATTGAT | |
| | AGCTTGTGCCTGTCCCTGAGTCCCAGTCCATCACGAGCAGCTGGTTTCTAAGATG | |
| | CTATTTCCCGTATAAAGCATGAGACCGTGACTTGCCAGCCCCACAGAGCCCCGCC | |
| | CTTGTCCATCACTGGCATCTGGACTCCAGCCTGGGTTGGGGCAAAGAGGGAAATG | |
| | AGATCATGTCCTAACCCTGATCCTCTTGTCCCACAGATATCCAGAACCCTGACCC | |
| | TGCCGTGTACCAGCTGAGAGACTCTAAATCCAGTGACAAGTCTGTCTGCCTATTC | |
| | ACCGATGGATCCGGATCTGCGATCGCTCCGGTGCCCGTCAGTGGGCAGAGCGCAC | |
| | ATCGCCCACAGTCCCCGAGAAGTTGGGGGGAGGGGTCGGCAATTGAACCGGTGCC | |
| | TAGAGAAGGTGGCGCGGGGTAAACTGGGAAAGTGATGTCGTGTACTGGCTCCGCC | |
| | TTTTTCCCGAGGGTGGGGGAGAACCGTATATAAGTGCAGTAGTCGCCGTGAACGT | |
| | TCTTTTTCGCAACGGGTTTGCCGCCAGAACACAGCTGAAGCTTCGAGGGGCTCGC | |
| | ATCTCTCCTTCACGCGCCCGCCGCCCTACCTGAGGCCGCCATCCACGCCGGTTGA | |
| | GTCGCGTTCTGCCGCCTCCCGCCTGTGGTGCCTCCTGAACTGCGTCCGCCGTCTA | |
| | GGTAAGTTTAAAGCTCAGGTCGAGACCGGGCCTTTGTCCGGCGCTCCCTTGGAGC | |
| | CTACCTAGACTCAGCCGGCTCTCCACGCTTTGCCTGACCCTGCTTGCTCAACTCT | |
| | ACGTCTTTGTTTCGTTTTCTGTTCTGCGCCGTTACAGATCCAAGCTGTGACCGGC | |
| | GCCTACGGCTAGCGCCACCATGCCGCTGCTGCTACTGCTGCCCCTGCTGTGGGCA | |
| | GGGGCTCTAGCTCAAGTGCAGCTGCAGGAGTCGGGCCCTGGCCTGGTGAAACCGT | |
| | CTGAGACCCTCAGCTTGACCTGCACTGTCTCCGGCGGGTCCATCTCTAGCTATTA | |
| | TTGGTCATGGATCCGGCAGCCCCCAGGCAAAGGACTCGAGTGGATCGGCTACATT | |
| | AACAACTCCGGGAGCACCAATTACAACCCTTCGCTAAAGTCTCGTGTGACCATCT | |
| | CAGTCGACACTTCCAAGAACCAGITTTCGTTAAAGCTGAGCTCTGTCACCGCTGC | |
| | GGACACCGCCGTGTACTATTGTGCCCGTGAGGATGATTACTGGGGACAGGGAACC | |
| | CTGGTTACAGTATCGTCCGGCGGAGGTGGTTCGGACATCCAGATGACCCAGACCA | |
| | CGTCCTCCTTGTCGGCTTCCCTGGGGGACCGCGTGACGATCTCATGCCGAGCAAG | |
| | CCAGGACATTTCTAAGTACCTCAATTGGTACCAGCAGAAGCCCGACGGCACCGTC | |
| | AAGCTGCTGATTTACCATACCTCCCGACTTCACTCTGGGGTGCCCTCTCGCTTCT | |
| | CCGGCTCTGGGTCAGGCACCGACTACTCCCTCACTATATCCAACCTGGAGCAGGA | |
| | AGATATCGCCACCTACTTCTGTCAGCAGGGCAACACGCTGCCGTACACCTTCGGC | |
| | GGCGGCACAAAACTGGAGATCACCGGAAGCACCAGTGGGTCCGGCAAGCCAGGCT | |
| | CTGGGGAGGGTTCGACCAAGGGCGAGGTGAAGCTGCAGGAGTCCGGCCCCGGACT | |
| | TGTGGCTCCTAGCCAGTCTCTATCTGTGACGTGCACAGTTTCGGGAGTGAGCCTG | |
| | CCCGACTACGGCGTCAGCTGGATCCGCCAGCCACCGCGCAAGGGACTGGAGTGGC | |
| | TGGGTGTGATCTGGGGCTCGGAGACCACCTACTACAATTCCGCGCTGAAGTCCCG | |
| | CCTCACTATCATCAAGGACAACTCTAAAAGCCAGGTGTTCCTGAAGATGAACAGT | |
| | TTGCAGACTGATGACACTGCCATCTACTACTGTGCCAAGCACTACTACTACGGCG | |
| | GTTCCTATGCCATGGATTATTGGGGCCAGGGCACATCGGTGACCGTGTCCAGTGG | |
| | AGGTGGCGGCAGCGAGATCGTCATGACCCAGAGCCCTGCGACCCTTTCGCTCTCC | |
| | CCGGGCGAGCGCGCTACTCTCTCTTGCCGCGCGAGTCAAATTATCTCCTCAAGCT | |
| | ACCTGAGCTGGTACCAGCAGAAGCCTGGTCAGGCCCCTAGACTGCTGATCTATGG | |
| | CGCATCCACCCGCGCGACCGGCATTCCCGCTCGTTTTTCCGGCTCTGGATCCGGA | |
| | ACGGACTTCACACTGACTATAAGCTCTCTTCAGCCGGAAGATTTCGCCGTATACT | |
| | ACTGTCAACAGGACTACGACTTTCCCTTCACCTTCGGCCCCGGCACCAAGGTGGA | |
| | CATCAAACGTGAGTCTAAATACGGACCGCCTTGTCCTCCTTGTCCCATGTTCTGG | |
| | GTGCTCGTGGTCGTTGGCGGAGTGCTGGCCTGTTACTCCCTGCTGGTTACCGTGG | |
| | CCTTCATCATCTTTTGGGTCAAGCGGGGCAGAAAGAAGCTGCTCTACATCTTCAA | |
| | GCAGCCCTTCATGCGGCCCGTGCAGACCACACAAGAGGAAGATGGCTGCTCCTGC | |
| | CGATTCCCCGAGGAAGAAGAAGGCGGCTGCGAGCTGAGAGTGAAGTTCAGCAGAT | |
| | CCGCCGACGCTCCAGCCTATCAGCAGGGCCAAAACCAGCTGTACAACGAGCTGAA | |
| | CCTGGGGAGAAGAGAAGAGTACGACGTGCTGGATAAGCGGAGAGGCAGAGATCCT | |
| | GAAATGGGCGGCAAGCCCAGACGGAAGAATCCTCAAGAGGGCCTGTATAATGAGC | |
| | TGCAGAAAGACAAGATGGCCGAGGCCTACAGCGAGATCGGAATGAAGGGCGAGCG | |
| | CAGAAGAGGCAAGGGACACGATGGACTGTACCAGGGCCTGAGCACCGCCACCAAG | |
| | GATACCTATGACGCACTGCACATGCAGGCCCTGCCACCTAGATGAGTCGACTGCT | |
| | TTATTTGTGAAATTTGTGATGCTATTGCTTTATTTGTAACCATTATAAGCTGCAA | |
| | TAAACAAGTTAACAACAACAATTGCATTCATTTTATGTTTCAGGTTCAGGGGGAG | |
| | GTGTGGGAGGTTTTTTAAAGGGCCCTTTGATTCTCAAACAAATGTGTCACAAAGT | |
| | AAGGATTCTGATGTGTATATCACAGACAAAACTGTGCTAGACATGAGGTCTATGG | |
| | ACTTCAAGAGCAACAGTGCTGTGGCCTGGAGCAACAAATCTGACTTTGCATGTGC | |
| | AAACGCCTTCAACAACAGCATTATTCCAGAAGACACCTTCTTCCCCAGCCCAGGT | |
| | AAGGGCAGCTTTGGTGCCTTCGCAGGCTGTTTCCTTGCTTCAGGAATGGCCAGGT | |
| | TCTGCCCAGAGCTCTGGTCAATGATGTCTAAAACTCCTCTGATTGGTGGTCTCGG | |
| | CCTTATCCATTGCCACCAAAACCCTCTTTTTACTAAGAAACAGTGAGCCTTGTTC | |
| | TGGCAGTCCAGAGAATGACACGGGAAAAAAGCAGATGAAGAGAAGGTGGCAGGAG | |
| | AGGGCACGTGGCCCAGCCTCAGTCTCTCCAACTGAGTTCCTGCCTGCCTGCCTTT | |
| | GCTCAGACTGTTTGCCCCTTACTGCTCTTCTAGGCCTCATTCTAAGCCCCTTCTC | |
| | CAAGTTGCCTCTCCTTATTTCTCCCTGTCTGCCAAAAAATCTTTCCCAGCTCACT | |
| | AAGTCAGTCTCACGCAGTCACTCATTAACCCACCAATCACTGATTGTG | |
| 130 | MPLLLLLPLLWAGALAQVQLQESGPGLVKPSETLSLTCTVSGGSISSYYWSWIRQ | JH2 |
| | PPGKGLEWIGYINNSGSTNYNPSLKSRVTISVDTSKNQFSLKLSSVTAADTAVYY | CAR/LoD/b |
| | CAREDDYWGQGTLVTVSSGGGGSDIQMTQTTSSLSASLGDRVTISCRASQDISKY | (amino acid |
| | LNWYQQKPDGTVKLLIYHTSRLHSGVPSRFSGSGSGTDYSLTISNLEQEDIATYF | sequence) |
| | CQQGNTLPYTFGGGTKLEITGSTSGSGKPGSGEGSTKGEVKLQESGPGLVAPSQS | |
| | LSVTCTVSGVSLPDYGVSWIRQPPRKGLEWLGVIWGSETTYYNSALKSRLTIIKD | |
| | NSKSQVFLKMNSLQTDDTAIYYCAKHYYYGGSYAMDYWGQGTSVTVSSGGGGSEI | |
| | VMTQSPATLSLSPGERATLSCRASQIISSSYLSWYQQKPGQAPRLLIYGASTRAT | |
| | GIPARFSGSGSGTDFTLTISSLQPEDFAVYYCQQDYDFPFTFGPGTKVDIKRESK | |
| | YGPPCPPCPMFWVLVVVGGVLACYSLLVTVAFIIFWVKRGRKKLLYIFKQPFMRP | |
| | VQTTQEEDGCSCRFPEEEEGGCELRVKFSRSADAPAYQQGQNQLYNELNLGRREE | |
| | YDVLDKRRGRDPEMGGKPRRKNPQEGLYNELQKDKMAEAYSEIGMKGERRRGKGH | |
| | DGLYQGLSTATKDTYDALHMQALPPR | |

1. A bispecific chimeric antigen receptor (CAR) comprising an extracellular binding domain, a spacer, a transmembrane domain, and an intracellular signaling domain, wherein the extracellular binding domain comprises:
a B-cell activating factor receptor (BAFF-R)-binding domain that binds to BAFF-R comprising a heavy chain variable (V_{H}) region and a light chain variable (V_{L}) region; and
a CD19-binding domain that binds to CD19 comprising a V_{H} region and a V_{L} region, wherein the extracellular binding domain comprises in order from the amino- to carboxy-terminus:
(i) the V_{H} region of the BAFF-R-binding domain, the V_{L} region of the CD19-binding domain, the V_{H} region of the CD19-binding domain, and the V_{L} region of the BAFF-R binding domain;
(ii) the V_{L} region of the BAFF-R-binding domain, the V_{L} region of the CD19-binding domain, the V_{H} region of the CD19-binding domain, and the V_{H} region of the BAFF-R binding domain;
(iii) the V_{H} region of the BAFF-R-binding domain, the V_{H} region of the CD19-binding domain, the V_{L} region of the CD19-binding domain, and the V_{L} region of the BAFF-R binding domain; or
(iv) the V_{L} region of the BAFF-R-binding domain, the V_{H} region of the CD19-binding domain, the V_{L} region of the CD19-binding domain, and the V_{H} region of the BAFF-R binding domain.

2. The bispecific CAR of claim 1, wherein the extracellular binding domain comprises in order from amino- to carboxy-terminus: the V_{L} region of the BAFF-R-binding domain, the V_{L} region of the CD19-binding domain, the V_{H} region of the CD19-binding domain, and the V_{H} region of the BAFF-R-binding domain.

3. The bispecific CAR of claim 1, wherein the extracellular binding domain comprises in order from amino- to carboxy-terminus: the V_{H} region of the BAFF-R-binding domain, the V_{L} region of the CD19-binding domain, the V_{H} region of the CD19-binding domain, and the V_{L} region of the BAFF-R-binding domain.

4. The bispecific CAR of any one of claims 1-3, wherein:
(i) the V_{H} region of the BAFF-R-binding domain comprises a CDR-H1, a CDR-H2 and a CDR-H3 each comprising a sequence that is contained within SEQ ID NO:1, and the V_{L} region of the BAFF-R-binding domain comprises a CDR-L1, a CDR-L2 and a CDR-L3 each comprising a sequence that is contained within SEQ ID NO:2;
(ii) the V_{H} region of the BAFF-R-binding domain comprises a CDR-H1, a CDR-H2 and a CDR-H3 each comprising a sequence that is contained within SEQ ID NO:3, and the V_{L} region of the BAFF-R-binding domain comprises a CDR-L1, a CDR-L2 and a CDR-L3 each having a sequence that is contained within SEQ ID NO:4;
(iii) the V_{H} region of the BAFF-R-binding domain comprises a CDR-H1, a CDR-H2 and a CDR-H3 each having a sequence that is contained within SEQ ID NO:5, and the V_{L} region of the BAFF-R-binding domain comprises a CDR-L1, a CDR-L2 and a CDR-L3 each comprising a sequence that is contained within SEQ ID: NO 6;
(iv) the V_{H} region of the BAFF-R-binding domain comprises a CDR-H1, a CDR-H2 and a CDR-H3 each comprising a sequence that is contained within SEQ ID NO:7, and the V_{L} region of the BAFF-R-binding domain comprises a CDR-L1, a CDR-L2 and a CDR-L3 each comprising a sequence that is contained within SEQ ID: NO 8; or
(v) the V_{H} region of the BAFF-R-binding domain comprises a CDR-H1, a CDR-H2 and a CDR-H3 each comprising a sequence that is contained within SEQ ID NO:9, and the V_{L} region of the BAFF-R-binding domain comprises a CDR-L1, a CDR-L2 and a CDR-L3 each comprising a sequence that is contained within SEQ ID: NO 10.

5. The bispecific CAR of any one of claims 1-4, wherein each CDR is defined in accordance with the Kabat definition, the Chothia definition, the combination of the Kabat and the Chothia definition, the AbM definition, or the contact definition.

6. A bispecific CAR comprising an extracellular binding domain, a transmembrane domain, a spacer, and an intracellular signaling domain, wherein the extracellular binding domain comprises:
a B-cell activating factor receptor (BAFF-R)-binding domain that binds to BAFF-R comprising a heavy chain variable (V_{H}) region and a light chain variable (V_{L}) region; and
a CD19-binding domain that binds to CD19 comprising a V_{H} region and a V_{L} region, wherein: (i) the V_{H} region of the BAFF-R-binding domain comprises a CDR-H1, a CDR-H2 and a CDR-H3 each comprising a sequence that is contained within SEQ ID NO:1, and the V_{L} region of the BAFF-R-binding domain comprises a CDR-L1, a CDR-L2 and a CDR-L3 each comprising a sequence that is contained within SEQ ID NO:2;
(ii) the V_{H} region of the BAFF-R-binding domain comprises a CDR-H1, a CDR-H2 and a CDR-H3 each comprising a sequence that is contained within SEQ ID NO:3, and the V_{L} region of the BAFF-R-binding domain comprises a CDR-L1, a CDR-L2 and a CDR-L3 each comprising a sequence that is contained within SEQ ID NO:4;
(iii) the V_{H} region of the BAFF-R-binding domain comprises a CDR-H1, a CDR-H2 and a CDR-H3 each comprising a sequence that is contained within SEQ ID NO:5, and the V_{L} region of the BAFF-R-binding domain comprises CDR-L1, CDR-L2 and CDR-L3 each comprising a sequence that is contained within SEQ ID: NO 6;
(iv) the V_{H} region of the BAFF-R-binding domain comprises a CDR-H1, a CDR-H2 and a CDR-H3 each comprising a sequence that is contained within SEQ ID NO:7, and the V_{L} region of the BAFF-R-binding domain comprises a CDR-L1, a CDR-L2 and a CDR-L3 each comprising a sequence that is contained within SEQ ID: NO 8; or
(v) the V_{H} region of the BAFF-R-binding domain comprises a CDR-H1, a CDR-H2 and a CDR-H3 each comprising a sequence that is contained within SEQ ID NO:9, and the V_{L} region of the BAFF-R-binding domain comprises a CDR-L1, a CDR-L2 and a CDR-L3 each comprising a sequence that is contained within SEQ ID: NO 10.

7. The bispecific CAR of claim 6, wherein each CDR is defined in accordance with the Kabat definition, the Chothia definition, the combination of the Kabat and the Chothia definition, the AbM definition, or the contact definition.

8. The bispecific CAR of any one of claims 1-7, wherein:
(i) the V_{H} region of the BAFF-R-binding domain comprises CDR-H1, CDR-H2 and CDR-H3 sequences set forth in SEQ ID NOS: 16, 17, and 18, respectively; and the V_{L} region of the BAFF-R-binding domain comprises CDR-L1, CDR-L2 and CDR-L3 sequences set forth in SEQ ID NOS: 19, 20, and 21, respectively;
(ii) the V_{H} region of the BAFF-R-binding domain comprises CDR-H1, CDR-H2 and CDR-H3 sequences set forth in SEQ ID NOS: 22, 23, and 24, respectively; and the V_{L} region of the BAFF-R-binding domain comprises CDR-L1, CDR-L2 and CDR-L3 sequences set forth in SEQ ID NOS: 25, 26, and 27, respectively;
(iii) the V_{H} region of the BAFF-R-binding domain comprises CDR-H1, CDR-H2 and CDR-H3 sequences set forth in SEQ ID NOS: 28, 29, and 30, respectively; and the V_{L} region of the BAFF-R-binding domain comprises CDR-L1, CDR-L2 and CDR-L3 sequences set forth in SEQ ID NOS: 31, 26, and 27, respectively;
(iv) the V_{H} region of the BAFF-R-binding domain comprises CDR-H1, CDR-H2 and CDR-H3 sequences set forth in SEQ ID NOS: 22, 32 and 24, respectively; and the V_{L} region of the BAFF-R-binding domain comprises CDR-L1, CDR-L2 and CDR-L3 sequences set forth in SEQ ID NOS: 33, 26, and 34, respectively; or
(v) the V_{H} region of the BAFF-R-binding domain comprises CDR-H1, CDR-H2 and CDR-H3 sequences set forth in SEQ ID NOS: 35, 36, and 37, respectively; and the V_{L} region of the BAFF-R-binding domain comprises CDR-L1, CDR-L2 and CDR-L3 sequences set forth in SEQ ID NOS: 38, 39, and 40, respectively.

9. A bispecific CAR comprising an extracellular binding domain, a transmembrane domain, a spacer, and an intracellular signaling domain, wherein the extracellular binding domain comprises:
a B-cell activating factor receptor (BAFF-R)-binding domain that binds to BAFF-R comprising a heavy chain variable (V_{H}) region and a light chain variable (V_{L}) region; and
a CD19-binding domain that binds to CD19 comprising a V_{H} region and a V_{L} region, wherein:
(i) the V_{H} region of the BAFF-R-binding domain comprises CDR-H1, CDR-H2 and CDR-H3 sequences set forth in SEQ ID NOS: 16, 17, and 18, respectively; and the V_{L} region of the BAFF-R-binding domain comprises CDR-L1, CDR-L2 and CDR-L3 sequences set forth in SEQ ID NOS: 19, 20, and 21, respectively;
(ii) the V_{H} region of the BAFF-R-binding domain comprises CDR-H1, CDR-H2 and CDR-H3 sequences set forth in SEQ ID NOS: 22, 23, and 24, respectively; and the V_{L} region of the BAFF-R-binding domain comprises CDR-L1, CDR-L2 and CDR-L3 sequences set forth in SEQ ID NOS: 25, 26, and 27, respectively;
(iii) the V_{H} region of the BAFF-R-binding domain comprises CDR-H1, CDR-H2 and CDR-H3 sequences set forth in SEQ ID NOS: 28, 29, and 30, respectively; and the V_{L} region of the BAFF-R-binding domain comprises CDR-L1, CDR-L2 and CDR-L3 sequences set forth in SEQ ID NOS: 31, 26, and 27, respectively;
(iv) the V_{H} region of the BAFF-R-binding domain comprises CDR-H1, CDR-H2 and CDR-H3 sequences set forth in SEQ ID NOS: 22, 32 and 24, respectively; and the V_{L} region of the BAFF-R-binding domain comprises CDR-L1, CDR-L2 and CDR-L3 sequences set forth in SEQ ID NOS: 33, 26, and 34, respectively; or
(v) the V_{H} region of the BAFF-R-binding domain comprises CDR-H1, CDR-H2 and CDR-H3 sequences set forth in SEQ ID NOS: 35, 36, and 37, respectively; and the V_{L} region of the BAFF-R-binding domain comprises CDR-L1, CDR-L2 and CDR-L3 sequences set forth in SEQ ID NOS: 38, 39, and 40, respectively.

10. The bispecific CAR of claims 6-9, wherein the extracellular binding domain comprises in order from the amino- to carboxy-terminus:
(i) the V_{H} region of the BAFF-R-binding domain, the V_{L} region of the CD19-binding domain, the V_{H} region of the CD19-binding domain, and the V_{L} region of the BAFF-R binding domain;
(ii) the V_{L} region of the BAFF-R-binding domain, the V_{L} region of the CD19-binding domain, the V_{H} region of the CD19-binding domain, and the V_{H} region of the BAFF-R binding domain;
(iii) the V_{H} region of the BAFF-R-binding domain, the V_{H} region of the CD19-binding domain, the V_{L} region of the CD19-binding domain, and the V_{L} region of the BAFF-R-binding domain;
(iv) the V_{L} region of the BAFF-R-binding domain, the V_{H} region of the CD19-binding domain, the V_{L} region of the CD19-binding domain, and the V_{H} region of the BAFF-R binding domain;
(v) the V_{L} region of the CD19-binding domain, the V_{H} region of the BAFF-R-binding domain, the V_{L} region of the BAFF-R-binding domain, and the V_{H} region of the CD19-binding domain;
(vi) the V_{H} region of the CD19-binding domain, the V_{H} region of the BAFF-R-binding domain, the V_{L} region of the BAFF-R-binding domain, and the V_{L} region of the CD19-binding domain;
(vii) the V_{L} region of the CD19-binding domain, the V_{H} region of the CD19-binding domain, the V_{H} region of the BAFF-R-binding domain, and the V_{L} region of the BAFF-R-binding domain;
(viii) the V_{L} region of the CD19-binding domain, the V_{H} region of the CD19-binding domain, the V_{L} region of the BAFF-R-binding domain, and the V_{H} region of the BAFF-R-binding domain;
(ix) the V_{H} region of the CD19-binding domain, the V_{L} region of the CD19-binding domain, the V_{H} region of the BAFF-R-binding domain, and the V_{L} region of the BAFF-R-binding domain;
(x) the V_{H} region of the CD19-binding domain, the V_{L} region of the CD19-binding domain, the V_{L} region of the BAFF-R-binding domain, and the V_{H} region of the BAFF-R-binding domain;
(xi) the V_{L} region of the BAFF-R-binding domain, the V_{H} region of the BAFF-R-binding domain, the V_{H} region of the CD19-binding domain, and the V_{L} region of the CD19-binding domain;
(xii) the V_{L} region of the BAFF-R-binding domain, the V_{H} region of the BAFF-R-binding domain, the V_{L} region of the CD19-binding domain, and the V_{H} region of the CD19-binding domain;
(xiii) the V_{H} region of the BAFF-R-binding domain, the V_{L} region of the BAFF-R-binding domain, the V_{H} region of the CD19-binding domain, and the V_{L} region of the CD19-binding domain;
(xiv) the V_{H} region of the BAFF-R-binding domain, the V_{L} region of the BAFF-R-binding domain, the V_{L} region of the CD19-binding domain, and the V_{H} region of the CD19-binding domain;
(xv) the V_{L} region of the CD19-binding domain, the V_{L} region of the BAFF-R-binding domain, the V_{H} region of the BAFF-R-binding domain, and the V_{H} region of the CD19-binding domain; or
(xvi) the V_{H} region of the CD19-binding domain, the V_{L} region of the BAFF-R-binding domain, the V_{H} region of the BAFF-R-binding domain, and the V_{L} region of the CD19-binding domain.

11. The bispecific CAR of any of claims 1-10, wherein:
(i) the V_{H} region of the BAFF-R-binding domain comprises the sequence set forth in, or a sequence that is at least 85%, 86%, 87%, 88%, 89%, 90%, 91%, 92%, 93%, 94%, 95%, 96%, 97%, 98% or 99% identical to, SEQ ID NO:1, and the V_{L} region of the BAFF-R-binding domain comprises the sequence set forth in, or a sequence that is at least 85%, 86%, 87%, 88%, 89%, 90%, 91%, 92%, 93%, 94%, 95%, 96%, 97%, 98% or 99% identical to, SEQ ID NO:2;
(ii) the V_{H} region of the BAFF-R-binding domain comprises the sequence set forth in, or a sequence that is at least 85%, 86%, 87%, 88%, 89%, 90%, 91%, 92%, 93%, 94%, 95%, 96%, 97%, 98% or 99% identical to, SEQ ID NO:3, and the V_{L} region of the BAFF-R-binding domain comprises the sequence set forth in, or a sequence that is at least 85%, 86%, 87%, 88%, 89%, 90%, 91%, 92%, 93%, 94%, 95%, 96%, 97%, 98% or 99% identical to, SEQ ID NO:4;
(iii) the V_{H} region of the BAFF-R-binding domain comprises the sequence set forth in, or a sequence that is at least 85%, 86%, 87%, 88%, 89%, 90%, 91%, 92%, 93%, 94%, 95%, 96%, 97%, 98% or 99% identical to, SEQ ID NO:5, and the V_{L} region of the BAFF-R-binding domain comprises the sequence set forth in, or a sequence that is at least 85%, 86%, 87%, 88%, 89%, 90%, 91%, 92%, 93%, 94%, 95%, 96%, 97%, 98% or 99% identical to, SEQ ID NO:6;
(iv) the V_{H} region of the BAFF-R-binding domain comprises the sequence set forth in, or a sequence that is at least 85%, 86%, 87%, 88%, 89%, 90%, 91%, 92%, 93%, 94%, 95%, 96%, 97%, 98% or 99% identical to, SEQ ID NO:7, and the V_{L} region of the BAFF-R-binding domain comprises the sequence set forth in, or a sequence that is at least 85%, 86%, 87%, 88%, 89%, 90%, 91%, 92%, 93%, 94%, 95%, 96%, 97%, 98% or 99% identical to, SEQ ID NO:8; or
(v) the V_{H} region of the BAFF-R-binding domain comprises the sequence set forth in, or a sequence that is at least 85%, 86%, 87%, 88%, 89%, 90%, 91%, 92%, 93%, 94%, 95%, 96%, 97%, 98% or 99% identical to, SEQ ID NO:9, and the V_{L} region of the BAFF-R-binding domain comprises the sequence set forth in, or a sequence that is at least 85%, 86%, 87%, 88%, 89%, 90%, 91%, 92%, 93%, 94%, 95%, 96%, 97%, 98% or 99% identical to, SEQ ID NO:10.

12. The bispecific CAR of any of claims 1-11, wherein:
(i) the V_{H} region of the BAFF-R-binding domain comprises the sequence set forth in SEQ ID NO:1, and the V_{L} region of the BAFF-R-binding domain comprises the sequence set forth in SEQ ID NO:2;
(ii) the V_{H} of the BAFF-R-binding domain comprises the sequence set forth in SEQ ID NO: 3, and the V_{L} region of the BAFF-R-binding domain comprises the sequence set forth in SEQ ID NO:4;
(iii) the V_{H} region of the BAFF-R-binding domain comprises the sequence set forth in SEQ ID NO:5, and the V_{L} region of the BAFF-R-binding domain comprises the sequence set forth in SEQ ID NO:6;
(iv) the V_{H} region of the BAFF-R-binding domain comprises the sequence set forth in SEQ ID NO:7, and the V_{L} region of the BAFF-R-binding domain comprises the sequence set forth in SEQ ID NO:8; or
(v) the V_{H} region of the BAFF-R-binding domain comprises the sequence set forth in SEQ ID NO:9, and the V_{L} region of the BAFF-R-binding domain comprises the sequence set forth in SEQ ID NO:10.

13. The bispecific CAR of any of claims 1-12, wherein the extracellular binding domain comprises, from amino to carboxy terminus: the V_{H} region of the BAFF-R-binding domain, the V_{L} region of the CD19-binding domain, the V_{H} region of the CD19-binding domain, and the V_{L} region of the BAFF-R-binding domain.

14. The bispecific CAR of any of claims 1-12, wherein the extracellular binding domain comprises, from amino to carboxy terminus: the V_{L} region of the BAFF-R-binding domain, the V_{L} region of the CD19-binding domain, the V_{H} region of the CD19-binding domain, and the V_{H} region of the BAFF-R-binding domain.

15. The bispecific CAR of any of claims 6-12, wherein the extracellular binding domain comprises, from amino to carboxy terminus: the V_{L} region of the CD19-binding domain, the V_{H} region of the BAFF-R-binding domain, the V_{L} region of the BAFF-R-binding domain, and the V_{H} region of the CD19-binding domain.

16. The bispecific CAR of any of claims 6-12, wherein the extracellular binding domain comprises, from amino to carboxy terminus: the V_{H} region of the CD19-binding domain, the V_{H} region of the BAFF-R-binding domain, the V_{L} region of the BAFF-R-binding domain, and the V_{L} region of the CD19-binding domain.

17. The bispecific CAR of any of claims 6-12, wherein the extracellular binding domain comprises, from amino to carboxy terminus: the V_{L} region of the CD19-binding domain, the V_{H} region of the CD19-binding domain, the V_{H} region of the BAFF-R-binding domain, and the V_{L} region of the BAFF-R-binding domain.

18. The bispecific CAR of any of claims 6-12, wherein the extracellular binding domain comprises, from amino to carboxy terminus: the V_{L} region of the CD19-binding domain, the V_{H} region of the CD19-binding domain, the V_{L} region of the BAFF-R-binding domain, and the V_{H} region of the BAFF-R-binding domain.

19. The bispecific CAR of any of claims 1-18, wherein the V_{H} region of the BAFF-R-binding domain comprises CDR-H1, CDR-H2 and CDR-H3 sequences set forth in SEQ ID NOS: 22, 23, and 24, respectively; and the V_{L} region of the BAFF-R-binding domain comprises CDR-L1, CDR-L2 and CDR-L3 sequences set forth in SEQ ID NOS: 25, 26, and 27, respectively.

20. The bispecific CAR of any of claims 1-19, wherein the V_{H} region of the BAFF-R-binding domain comprises the sequence set forth in, or a sequence that is at least 85%, 86%, 87%, 88%, 89%, 90%, 91%, 92%, 93%, 94%, 95%, 96%, 97%, 98% or 99% identical to, SEQ ID NO:3, and the V_{L} region of the BAFF-R-binding domain comprises the sequence set forth in, or a sequence that is at least 85%, 86%, 87%, 88%, 89%, 90%, 91%, 92%, 93%, 94%, 95%, 96%, 97%, 98% or 99% identical to, SEQ ID NO:4.

21. The bispecific CAR of any of claims 1-20, wherein the V_{H} of the BAFF-R-binding domain comprises the sequence set forth in SEQ ID NO:3, and the V_{L} region of the BAFF-R-binding domain comprises the sequence set forth in SEQ ID NO:4.

22. The bispecific CAR of any of claims 1-18, wherein the V_{H} region of the BAFF-R-binding domain comprises the sequence set forth in, or a sequence that is at least 85%, 86%, 87%, 88%, 89%, 90%, 91%, 92%, 93%, 94%, 95%, 96%, 97%, 98% or 99% identical to, SEQ ID NO:5, and the V_{L} region of the BAFF-R-binding domain comprises the sequence set forth in, or a sequence that is at least 85%, 86%, 87%, 88%, 89%, 90%, 91%, 92%, 93%, 94%, 95%, 96%, 97%, 98% or 99% identical to, SEQ ID NO:6.

23. The bispecific CAR of any of claims 1-18 and 22, wherein the V_{H} region of the BAFF-R-binding domain comprises CDR-H1, CDR-H2 and CDR-H3 sequences set forth in SEQ ID NOS: 28, 29, and 30, respectively; and the V_{L} region of the BAFF-R-binding domain comprises CDR-L1, CDR-L2 and CDR-L3 sequences set forth in SEQ ID NOS: 31, 26, and 27, respectively.

24. The bispecific CAR of any of claims 1-18, 22, and 23, wherein the V_{H} region of the BAFF-R-binding domain comprises the sequence set forth in SEQ ID NO:5, and the V_{L} region of the BAFF-R-binding domain comprises the sequence set forth in SEQ ID NO:6.

25. The bispecific CAR of any of claims 1-18, wherein the V_{H} region of the BAFF-R-binding domain comprises CDR-H1, CDR-H2 and CDR-H3 sequences set forth in SEQ ID NOS: 16, 17, and 18, respectively; and the V_{L} region of the BAFF-R-binding domain comprises CDR-L1, CDR-L2 and CDR-L3 sequences set forth in SEQ ID NOS: 19, 20, and 21, respectively.

26. The bispecific CAR of any of claims 1-18 and 25, wherein the V_{H} region of the BAFF-R-binding domain comprises the sequence set forth in, or a sequence that is at least 85%, 86%, 87%, 88%, 89%, 90%, 91%, 92%, 93%, 94%, 95%, 96%, 97%, 98% or 99% identical to, SEQ ID NO:1, and the V_{L} region of the BAFF-R-binding domain comprises the sequence set forth in, or a sequence that is at least 85%, 86%, 87%, 88%, 89%, 90%, 91%, 92%, 93%, 94%, 95%, 96%, 97%, 98% or 99% identical to, SEQ ID NO:2.

27. The bispecific CAR of any of claims 1-18, 25, and 26, wherein the V_{H} region of the BAFF-R-binding domain comprises the sequence set forth in SEQ ID NO:1, and the V_{L} region of the BAFF-R-binding domain comprises the sequence set forth in SEQ ID NO:2.

28. The bispecific CAR of any of claims 1-18, wherein the V_{H} region of the BAFF-R-binding domain comprises CDR-H1, CDR-H2 and CDR-H3 sequences set forth in SEQ ID NOS: 22, 32 and 24, respectively; and the V_{L} region of the BAFF-R-binding domain comprises CDR-L1, CDR-L2 and CDR-L3 sequences set forth in SEQ ID NOS: 33, 26, and 34, respectively.

29. The bispecific CAR of any of claims 1-18 and 28, wherein the V_{H} region of the BAFF-R-binding domain comprises the sequence set forth in, or a sequence that is at least 85%, 86%, 87%, 88%, 89%, 90%, 91%, 92%, 93%, 94%, 95%, 96%, 97%, 98% or 99% identical to, SEQ ID NO:7, and the V_{L} region of the BAFF-R-binding domain comprises the sequence set forth in, or a sequence that is at least 85%, 86%, 87%, 88%, 89%, 90%, 91%, 92%, 93%, 94%, 95%, 96%, 97%, 98% or 99% identical to, SEQ ID NO:8.

30. The bispecific CAR of any of claims 1-18, 28, and 29, wherein the V_{H} region of the BAFF-R-binding domain comprises the sequence set forth in SEQ ID NO:7, and the V_{L} region of the BAFF-R-binding domain comprises the sequence set forth in SEQ ID NO:8.

31. The bispecific CAR of any of claims 1-18, wherein the V_{H} region of the BAFF-R-binding domain comprises CDR-H1, CDR-H2 and CDR-H3 sequences set forth in SEQ ID NOS: 35, 36, and 37, respectively; and the V_{L} region of the BAFF-R-binding domain comprises CDR-L1, CDR-L2 and CDR-L3 sequences set forth in SEQ ID NOS: 38, 39, and 40, respectively.

32. The bispecific CAR of any of claims 1-18 and 31, wherein the V_{H} region of the BAFF-R-binding domain comprises the sequence set forth in, or a sequence that is at least 85%, 86%, 87%, 88%, 89%, 90%, 91%, 92%, 93%, 94%, 95%, 96%, 97%, 98% or 99% identical to, SEQ ID NO:9, and the V_{L} region of the BAFF-R-binding domain comprises the sequence set forth in, or a sequence that is at least 85%, 86%, 87%, 88%, 89%, 90%, 91%, 92%, 93%, 94%, 95%, 96%, 97%, 98% or 99% identical to, SEQ ID NO:10.

33. The bispecific CAR of any of claims 1-18, 31, and 32, wherein the V_{H} region of the BAFF-R-binding domain comprises the sequence set forth in SEQ ID NO:9, and the V_{L} region of the BAFF-R-binding domain comprises the sequence set forth in SEQ ID NO: 10.

34. The bispecific CAR of any of claims 1-33, wherein the V_{H} region of the BAFF-R binding domain is joined to the V_{L} region of the BAFF-R binding domain via an intradomain linker.

35. The bispecific CAR of any one of claims 1-34, wherein the V_{H} region of the CD19-binding domain comprises a CDR-H1, a CDR-H2 and a CDR-H3 each comprising a sequence that is contained within SEQ ID NO:41, and the V_{L} region of the CD19-binding domain comprises a CDR-L1, a CDR-L2 and a CDR-L3 each comprising a sequence that is contained within SEQ ID: NO 42.

36. The bispecific CAR of claim 35, wherein each CDR is defined in accordance with the Kabat definition, the Chothia definition, the combination of the Kabat and the Chothia definition, the AbM definition, or the contact definition.

37. The bispecific CAR of any of claims 1-36, wherein the V_{H} region of the CD19-binding domain comprises CDR-H1, CDR-H2 and CDR-H3 sequences set forth in SEQ ID NOS: 41, 44 and 46, respectively; and the V_{L} region of the CD19-binding domain comprises CDR-L1, CDR-L2 and CDR-L3 sequences set forth in SEQ ID NOS: 47, 49, and 51, respectively.

38. The bispecific CAR of any one of claims 1-37, wherein
(i) the V_{H} region of the CD19-binding domain comprises the sequences set forth in, or a sequence that is at least 90%, 91%, 92%, 93%, 94%, 95%, 96%, 97%, 98% or 99% identical to, SEQ ID NO: 41; and
(ii) the V_{L} region of the CD19-binding domain comprises the sequences set forth in, or a sequence that is at least 90%, 91%, 92%, 93%, 94%, 95%, 96%, 97%, 98% or 99% identical to, SEQ ID NO:42.

39. The bispecific CAR of any one of claims 1-38, wherein the V_{H} region of the CD19-binding domain comprises the sequence set forth in SEQ ID NO: 41; and the V_{L} region of the CD19-binding domain comprises the sequence set forth in SEQ ID NO:42.

40. A bispecific CAR comprising an extracellular binding domain, a spacer, a transmembrane domain, and an intracellular signaling domain, wherein the extracellular binding domain comprises:
a B-cell activating factor receptor (BAFF-R)-binding domain that binds to BAFF-R comprising a heavy chain variable (V_{H}) region and a light chain variable (V_{L}) region; and
a CD19-binding domain that binds to CD19 comprising a V_{H} region and a V_{L} region, wherein the extracellular binding domain comprises in order from amino to carboxy terminus:
the V_{H} region of the BAFF-R-binding domain comprising the sequence set forth in SEQ ID NO: 3, the V_{L} region of the CD19-binding domain comprising the sequence set forth in SEQ ID NO: 42, the V_{H} region of the CD19-binding domain comprising the sequence set forth in SEQ ID NO: 41, and the V_{L} region of the BAFF-R-binding domain comprising the sequence set forth in SEQ ID NO: 4.

41. A bispecific CAR comprising an extracellular binding domain, a spacer, a transmembrane domain, and an intracellular signaling domain, wherein the extracellular binding domain comprises:
a BAFF-R-binding domain comprising V_{H} and V_{L}; and
a CD19-binding domain comprising V_{H} and V_{L},
wherein the extracellular binding domain comprises in order from amino to carboxy terminus:
the V_{L} region of the BAFF-R-binding domain comprising the sequence set forth in SEQ ID NO: 6, the V_{L} region of the CD19-binding domain comprising the sequence set forth in SEQ ID NO: 42, the V_{H} region of the CD19-binding domain comprising the sequence set forth in SEQ ID NO: 41, and the V_{H} region of the BAFF-R-binding domain comprising the sequence set forth in SEQ ID NO: 5.

42. The bispecific CAR of any of claims 1-41, wherein the V_{H} region of the CD19-binding domain is joined to the V_{L} region of the CD19-binding domain via an intradomain linker.

43. The bispecific CAR of claim 34 or claim 42, wherein the intradomain linker is a flexible linker.

44. The bispecific CAR of any of claims 34, 42, and 43, wherein the intradomain linker is 5 to 25 amino acids in length.

45. The bispecific CAR of any of claims 34 and 42-44, wherein the intradomain linker is 12 to 18 amino acids in length.

46. The bispecific CAR of any of claims 34, and 42-45, wherein the intradomain linker comprises the sequence set forth in SEQ ID NO:58.

47. The bispecific CAR of any of claims 34 and 42-45, wherein the intradomain linker comprises the sequence set forth in SEQ ID NO:59.

48. The bispecific CAR of any of claims 1-47, wherein the V_{H} region or the V_{L} region of the BAFF-R-binding domain are joined by an interdomain linker to the V_{H} region or the V_{L} region of the CD19-binding domain.

49. The bispecific CAR of any of claims 1-48, wherein:
(i) the V_{H} region of the BAFF-R binding domain is joined to the V_{L} region of the CD19-binding domain by an interdomain linker;
(i) the V_{H} region of the BAFF-R binding domain is joined to the V_{H} region of the CD19-binding domain by an interdomain linker;
(iii) the V_{L} region of the BAFF-R binding domain is joined to the V_{L} region of the CD19-binding domain by an interdomain linker; or
(iv) the V_{L} region of the BAFF-R binding domain is joined to the V_{H} region of the CD19-binding domain by an interdomain linker.

50. The bispecific CAR of claim 48 or claim 49, wherein the interdomain linker is a flexible peptide linker.

51. The bispecific CAR of any of claims 48-50, wherein the length of the interdomain linker is between 5 and 25 amino acids, inclusive.

52. The bispecific CAR of any of claims 48-51, wherein the length of the interdomain linker is between 5 and 15 amino acids, inclusive.

53. The bispecific CAR of any of claims 34 and 48-52, wherein the interdomain linker is a G4S linker (SEQ ID NO:60), a G4S2 linker (SEQ ID NO:61) or a (G4S)4 linker (SEQ ID NO:62).

54. The bispecific CAR of any one of claims 48-53, wherein the interdomain linker is set forth in SEQ ID NO: 60.

55. The bispecific CAR of any one of claims 48-54, wherein the interdomain linker is set forth in SEQ ID NO: 61.

56. The bispecific CAR of any of claims 1-55, wherein the spacer is interposed between the extracellular binding domain and the transmembrane domain.

57. The bispecific CAR of any one of claims 1-56, wherein the spacer comprises a hinge region sequence.

58. The bispecific CAR of any one of claims 1-57, wherein the spacer comprises a hinge region of an immunoglobulin or a variant thereof.

59. The bispecific CAR of claim 52, wherein the hinge region of an immunoglobulin is an IgG4 hinge region, optionally a human IgG4 hinge region, or a variant thereof.

60. The bispecific CAR of any one of claims 1-59, wherein the spacer comprises a variant IgG4 hinge region comprising substitution of amino acids CPSC to CPPC compared to the wild-type IgG4 hinge region.

61. The bispecific CAR of any one of claims 1-60, wherein the spacer is less than at or about 15 amino acids in length.

62. The bispecific CAR of any one of claims 1-61, wherein the spacer is between 12 and 15 amino acids in length.

63. The bispecific CAR of any one of claims 1-62, wherein the spacer comprises an amino acid sequence having at least at or about 85%, 86%, 87%, 88%, 89%, 90%, 91%, 92%, 93%, 94%, 95%, 96%, 97%, 98% or 99% identity to SEQ ID NO: 52, optionally wherein the spacer has the sequence set forth in SEQ ID NO:52.

64. The bispecific CAR of any one of claims 1-60, wherein the spacer is between 200 and 250 amino acids in length, or between 220 and 240 amino acids in length.

65. The bispecific CAR of any one of claims 1-60 and 64 wherein the spacer comprises a hinge region of an immunoglobulin, a CH2 region of an immunoglobulin or a chimeric CH2 region of two different immunoglobulins, and a CH3 region of an immunoglobulin.

66. The bispecific CAR of claim 65, wherein the spacer comprises an IgG4 hinge region or a variant thereof, a chimeric CH2 region comprising a portion of an IgG4 CH2 and a portion of an IgG2 CH2 (IgG2/4 CH2 region), and an IgG4 CH3 region.

67. The bispecific CAR of any one of claims 1-62 and 64-66, wherein the spacer comprises an amino acid sequence having at least at or about 85%, 86%, 87%, 88%, 89%, 90%, 91%, 92%, 93%, 94%, 95%, 96%, 97%, 98% or 99% identity to SEQ ID NO:53, optionally wherein the spacer has the sequence set forth in SEQ ID NO: 53.

68. The bispecific CAR of any one of claims 1-67 wherein the transmembrane domain comprises a transmembrane domain from CD28, optionally a human CD28.

69. The bispecific CAR of any one of claims 1-68, wherein the transmembrane domain is or comprises SEQ ID NO: 55 or an amino acid sequence having at least at or about 90%, 91%, 92%, 93%, 94%, 95%, 96%, 97%, 98% or 99% sequence identity to SEQ ID NO: 55.

70. The bispecific CAR of any one of claims 1-69, wherein the transmembrane domain is set forth in SEQ ID NO: 55.

71. The bispecific CAR of any one of claims 1-70, wherein the intracellular signaling domain comprises an intracellular signaling domain capable of inducing a primary activation signal in a T cell.

72. The bispecific CAR of claim 71, wherein the intracellular signaling domain is a domain from a T cell receptor (TCR) component and/or comprises an immunoreceptor tyrosine-based activation motif (ITAM).

73. The bispecific CAR of claim 71 or claim 72, wherein the intracellular signaling domain is a cytoplasmic signaling domain of a CD3-zeta (CD3ζ) chain, optionally a human CD3ζ chain.

74. The bispecific CAR of any one of claims 71-73, wherein the intracellular signaling domain comprises the sequence set forth in SEQ ID NO: 57, or an amino acid sequence having at least at or about 90%, 91%, 92%, 93%, 94%, 95%, 96%, 97%, 98% or 99% sequence identity to SEQ ID NO: 57.

75. The bispecific CAR of any one of claims 71-74, wherein the intracellular signaling domain is set forth in SEQ ID NO: 57.

76. The bispecific CAR of any one of claims 71-75, wherein the intracellular signaling region further comprises a costimulatory signaling region.

77. The bispecific CAR of claim 76, wherein the costimulatory signaling region is between the transmembrane region and the intracellular signaling domain.

78. The bispecific CAR of claim 76 or claim 77, wherein the costimulatory signaling region comprises an intracellular signaling domain of a T cell costimulatory molecule or a signaling portion thereof.

79. The bispecific CAR of any one of claims 76-78, wherein the costimulatory signaling region comprises an intracellular signaling domain of 4-1BB, optionally a human 4-1BB.

80. The bispecific CAR of any of claims 76-79, wherein the costimulatory signaling region comprises the sequence set forth in SEQ ID NO: 56 or an amino acid sequence having at least at or about 90%, 91%, 92%, 93%, 94%, 95%, 96%, 97%, 98% or 99% sequence identity to SEQ ID NO: 56.

81. The bispecific CAR of any of claims 76-80, wherein the costimulatory signaling region is set forth in SEQ ID NO: 56.

82. A bispecific CAR comprising the amino acid sequence set forth in SEQ ID NO: 94, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 94.

83. A bispecific CAR comprising the amino acid sequence set forth in SEQ ID NO: 95, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 95.

84. A bispecific CAR comprising the amino acid sequence set forth in SEQ ID NO: 96, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 96.

85. A bispecific CAR comprising the amino acid sequence set forth in SEQ ID NO: 97, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 97.

86. A bispecific CAR comprising the amino acid sequence set forth in SEQ ID NO: 98, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 98.

87. A bispecific CAR comprising the amino acid sequence set forth in SEQ ID NO: 99, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 99.

88. A bispecific CAR comprising the amino acid sequence set forth in SEQ ID NO: 100, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 100.

89. A bispecific CAR comprising the amino acid sequence set forth in SEQ ID NO: 101, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 101.

90. A bispecific CAR comprising the amino acid sequence set forth in SEQ ID NO: 102, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 102.

91. A bispecific CAR comprising the amino acid sequence set forth in SEQ ID NO: 103, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 103.

92. The bispecific CAR of any of claims 1-91, wherein the binding of the BAFF-R domain to BAFF in the presence of soluble BAFF is reduced no more than 10% relative to binding in the absence of soluble BAFF.

93. The bispecific CAR of any of claims 1, 3-13, 19-21, 35-40, 42-45, 47-54, and 56-92 wherein the binding of the CD19 domain to CD19 in the presence of FMC63 scFv is reduced no more than 30% relative to binding in the absence of FMC63 scFv.

94. A polynucleotide encoding the bispecific CAR of any one of claims 1-93.

95. The polynucleotide of claim 94, wherein the polynucleotide is optimized by splice site elimination.

96. The polynucleotide of claim 94 or claim 95, wherein the polynucleotide is codon-optimized for expression in a human cell.

97. A vector comprising the polynucleotide of any one of the claims 94-96.

98. The vector of claim 97, wherein the vector is a viral vector.

99. The vector of claim 98, wherein the viral vector is a retroviral vector (e.g., lentiviral vector).

100. A cell comprising the bispecific CAR of any one of claims 1-93.

101. A cell comprising the polynucleotide of any of claims 94-96 or the vector of any of claims 97-99.

102. The cell of claim 100 or 101, wherein the cell is an immune cell.

103. The cell of any one of claims 100-102, wherein the cell is a lymphocyte.

104. The cell of any one of claims 100-103, that is an NK cell or a T cell.

105. The cell of any one of claims 100-104, wherein the cell is a T cell.

106. The cell of any one of claims 100-105, wherein the T cell is a CD4+ T cell or a CD8+ T cell.

107. The cell of any one of claims 100-106, wherein the cell is a primary cell.

108. The cell of any one of claims 100-107, wherein the cell exhibits cytotoxic activity against CD19+ cells, BAFF-R+ cells, and CD19+/BAFF-R+ cells.

109. A composition comprising a plurality of cells of any one of claims 100-108.

110. The composition of claim 109, further comprising a pharmaceutically acceptable excipient.

111. The composition of claim 109 or 110, wherein the composition comprises CD4+ and CD8+ T cells.

112. The composition of claim 111, wherein the ratio of CD4+ to CD8+ T cells is from at or about 1:3 to 3:1, optionally at or about 1:2 to 2:1, optionally at or about 1:1.

113. The composition of any one of claims 109-112, wherein greater than at or about 90%, greater than at or about 95% or greater than at or about 98% of cells in the composition are CD3+ T cells.

114. The composition of any one of claims 109-113, wherein at least 40%, at least 50%, at least 60%, at least 70%, at least 80% or at least 90% of cells in the composition express the CAR.

115. The composition of any one of claims 109-114, wherein, among a plurality of the cells in the composition expressing the bispecific CAR, less than at or about 10%, at or about 9%, at or about 8%, at or about 7%, at or about 5%, at or about 4%, at or about 3%, at or about 2% or at or about 1% of the cells in the plurality exhibits tonic signaling and/or antigen independent activity or signaling.

116. The composition of any one of claims 109-115, wherein the composition comprises between about 1.0×10⁷ bispecific CAR-expressing T cells and 1.2×10⁹ bispecific CAR-expressing T cells, between about 1.0×10⁷ bispecific CAR-expressing T cells and 6.5×10⁸ bispecific CAR-expressing T cells, between about 1.5×10⁷ bispecific CAR-expressing T cells and 6.5×10⁸ bispecific CAR-expressing T cells, between about 1.5×10⁷ bispecific CAR-expressing T cells and 6.0×10⁸ bispecific CAR-expressing T cells, between about 2.5×10⁷ bispecific CAR-expressing T cells and 6.0×10⁸ bispecific CAR-expressing T cells, between about 5.0×10⁷ bispecific CAR-expressing T cells and 6.0×10⁸ bispecific CAR-expressing T cells, between about 1.25×10⁷ bispecific CAR-expressing T cells and 1.2×10⁹ bispecific CAR-expressing T cells, between about 1.5×10⁷ bispecific CAR-expressing T cells and 1.2×10⁹ bispecific CAR-expressing T cells, between about 5.0×10⁷ bispecific CAR-expressing T cells and 4.5×10⁸ bispecific CAR-expressing T cells, or between about 1.5×10⁸ bispecific CAR-expressing T cells and 3.0×10⁸ bispecific CAR-expressing T cells, each inclusive.

117. The composition of any one of claims 109-116, wherein the composition comprises at or about 1.5×10⁷, at or about 2.5×10⁷, at or about 5.0×10⁷, at or about 7.5×10⁷, at or about 1.5×10⁸, at or about 2.25×10⁸, at or about 3.0×10⁸, at or about 4.5×10⁸, at or about 6.0×10⁸, at or about 8.0×10⁸, or at or about 1.2×10⁹ bispecific CAR-expressing T cells.

118. A method of treating a disease or disorder in a subject, the method comprising administering the cell of any of claims 100-108 or the composition of any of claims 107-115 to a subject in need of treatment thereof.

119. A method of treatment, comprising administering the bispecific CAR of any of claims 1-93, the polynucleotide of any one of claims 94-96, or the vector of any one of claims 97-99 to a subject having a disease or disorder.

120. The method of claim 118 or claim 119, wherein the disease or disorder is a cancer or an autoimmune disease.

121. The method of claim 120, wherein the disease or disorder is a cancer.

122. The method of claim 120 or 121, wherein the cancer is a BAFF-R-expressing cancer, a CD19-expressing cancer, or a BAFF-R- and CD19-expressing cancer.

123. The method of any of claims 120-122, wherein the cancer is a lymphoma or a leukemia.

124. The method of claim 123, wherein the lymphoma is a large B cell lymphoma.

125. The method of claim 123, wherein the lymphoma is a non-Hodgkin lymphoma.

126. The method of claim 120, wherein the disease or disorder is a an autoimmune disease.

127. The cell of any of claims 100-108 or the composition of any of claims 109-117 for use in treating a disease or disorder.

128. Use of the cell of any of claims 100-108 or the composition of any of claims 109-117 for the manufacture of a medicament for treating a disease or disorder.

129. Use of the cell of any of claims 100-108 or the composition of any of claims 109-117 for the treatment of a disease or disorder.

130. The bispecific CAR of any one of claims 1-93, the polynucleotide of any one of claims 94-96, or the vector of any one of claims 97-99 for use in treating a disease or disorder.

131. Use of the bispecific CAR of any of claims 1-93, the polynucleotide of any one of claims 94-96, or the vector of any one of claims 97-99 for the manufacture of a medicament for treating a disease or disorder.

132. Use of the bispecific CAR of any of claims 1-93, the polynucleotide of any one of claims 94-96, or the vector of any one of claims 97-99 for the treatment of a disease or disorder.

133. The use of claim 126 or claim 127, wherein the disease or disorder is a cancer or an autoimmune disease.

134. The use of claim 133, wherein the disease or disorder is a cancer.

135. The use of claim 133 or 134, wherein the cancer is a BAFF-R-expressing cancer, a CD19-expressing cancer, or a BAFF-R- and CD19-expressing cancer.

136. The use of any of claims 133-135, wherein the cancer is a lymphoma or a leukemia.

137. The use of claim 136, wherein the lymphoma is a large B cell lymphoma.

138. The use of claim 136, wherein the lymphoma is a non-Hodgkin lymphoma.

139. The use of claim 133, wherein the disease or disorder is an autoimmune disease.

140. The use of claim 128 or 129, wherein the disease or disorder is a cancer or an autoimmune disease.

141. The use of claim 140, wherein the disease or disorder is a cancer.

142. The use of claim 140 or 141, wherein the cancer is a BAFF-R-expressing cancer, a CD19-expressing cancer, or a BAFF-R- and CD19-expressing cancer.

143. The use of any of claims 140-142, wherein the cancer is a lymphoma or a leukemia.

144. The use of claim 143, wherein the lymphoma is a large B cell lymphoma.

145. The use of claim 143, wherein the lymphoma is a non-Hodgkin lymphoma.

146. The use of claim 140, wherein the disease or disorder is an autoimmune disease.

147. A kit comprising the bispecific CAR of any one of claims 1-93, the polynucleotide of any one of claims 94-96, the vector of any one of claims 97-99, the cell of any one of claims 100-108, or the composition of any one of claims 109-117, and instructions for use, optionally wherein the instructions are for administering the bispecific CAR, the cell, or the composition, optionally in accord with the method, the cell, the composition, the bispecific CAR, the polynucleotide, or the vector for use or the use of any of claims 128-146.

148. An article of manufacture comprising the bispecific CAR of any one of claims 1-93, the polynucleotide of any one of claims 94-96, the vector of any one of claims 97-99, the cell of any one of claims 100-108, or the composition of any one of claims 109-117 or the kit of claim 147.

149. An antibody or antigen-binding portion thereof that binds B-cell activating factor receptor (BAFF-R), comprising a heavy chain variable region (V_{H}) and a light chain variable region (V_{L}), wherein
(i) V_{H} comprises CDR-H1, CDR-H2, CDR-H3 each having a sequence that is contained within SEQ ID NO:1, and the V_{L} region comprises a light chain complementarity determining region 1 (CDR-L1), a light chain complementarity determining region 2 (CDR-L2) and a light chain complementarity determining region 3 (CDR-L3) contained within SEQ ID NO: 2;
(ii) the V_{H} region comprises a CDR-H1, a CDR-H2 and a CDR-H3 contained within SEQ ID NO:3, and the V_{L} region comprises a CDR-L1, a CDR-L2 and a CDR-L3 contained within SEQ ID: NO 4;
(iii) the V_{H} region comprises a CDR-H1, a CDR-H2 and a CDR-H3 contained within SEQ ID NO:5, and the V_{L} region comprises a CDR-L1, a CDR-L2 and a CDR-L3 contained within SEQ ID: NO 6;
(iv) the V_{H} region comprises a CDR-H1, a CDR-H2 and a CDR-H3 contained within SEQ ID NO:7, and the V_{L} region comprises a CDR-L1, a CDR-L2 and a CDR-L3 contained
(v) V_{H} comprises a CDR-H1, a CDR-H2 and a CDR-H3 contained within SEQ ID NO: 9, and the V_{L} region comprises a CDR-L1, a CDR-L2 and a CDR-L3 contained within SEQ ID: NO 10.

150. The antibody or antigen-binding portion thereof of claim 149, wherein V_{H} comprises a CDR-H1, a CDR-H2, and a CDR-H3 each having a sequence that is contained within SEQ ID NO: 1, and V_{L} comprises a CDR-L1, a CDR-L2, and a CDR-L3 each having a sequence that is contained within SEQ ID: NO 2.

151. The antibody or antigen-binding portion thereof of claim 150, wherein V_{H} comprises a CDR-H1, a CDR-H2 and a CDR-H3 contained within SEQ ID NO: 3, and V_{L} comprises a CDR-L1, a CDR-L2 and a CDR-L3 contained within SEQ ID: NO 4.

152. The antibody or antigen-binding fragment thereof of claim 150, wherein the V_{H} region comprises a CDR-H1, a CDR-H2 and a CDR-H3 contained within SEQ ID NO: 5, and the V_{L} region comprises a CDR-L1, a CDR-L2 and a CDR-L3 contained within SEQ ID: NO 6.

153. The antibody or antigen-binding fragment thereof of claim 150, wherein the V_{H} region comprises a CDR-H1, a CDR-H2 and a CDR-H3 contained within SEQ ID NO: 7, and the V_{L} region comprises a CDR-L1, a CDR-L2 and a CDR-L3 contained within SEQ ID: NO 8.

154. The antibody or antigen-binding fragment thereof of claim 150, wherein the V_{H} region comprises a CDR-H1, a CDR-H2 and a CDR-H3 contained within SEQ ID NO: 9, and the V_{L} region comprises a CDR-L1, a CDR-L2 and a CDR-L3 contained within SEQ ID: NO 10.

155. The antibody or antigen-binding portion of any one of claims 150-154, wherein each CDR is defined in accordance with the Kabat definition, the Chothia definition, the combination of the Kabat and the Chothia definition, the AbM definition, or the contact definition.

156. The antibody or antigen-binding portion of any one of claims 149-154, wherein the antibody or antigen-binding portion thereof binds to BAFF-R with a K_{D} of about 10⁻⁷ M to about 10⁻¹¹ M.

157. An antibody or antigen-binding portion thereof that specifically binds BAFF-R, comprising V_{H} and V_{L}, wherein:
(i) V_{H} comprises a CDR-H1, a CDR-H2, and a CDR-H3 comprising the sequences set forth in SEQ ID NOS: 16, 17, and 18, respectively, and V_{L} comprises a CDR-L1, CDR-L2, and a CDR-L3 comprising the sequences set forth in SEQ ID NOS: 19, 20, and 21, respectively;
(ii) V_{H} comprises a CDR-H1, a CDR-H2, and a CDR-H3 comprising the sequences set forth in SEQ ID NOS: 22, 23, and 24, respectively, and V_{L} comprises a CDR-L1, a CDR-L2 and a CDR-L3 comprising the sequences set forth in SEQ ID NOS: 25, 26, and 27, respectively;
(iii) V_{H} comprises a CDR-H1, a CDR-H2, and a CDR-H3 comprising the sequences set forth in SEQ ID NOS: 28, 29, and 30, respectively, and V_{L} region comprises a CDR-L1, a CDR-L2 and a CDR-L3 comprising the sequences set forth in SEQ ID NOS: 31, 26, and 27, respectively;
(iv) V_{H} comprises a CDR-H1, a CDR-H2, and a CDR-H3 comprising the sequences set forth in SEQ ID NOS: 22, 32 and 24, respectively, and V_{L} comprises a CDR-L1, a CDR-L2 and a CDR-L3 comprising the sequences set forth in SEQ ID NOS: 33, 26, and 34, respectively; or
(v) V_{H} comprises a CDR-H1, a CDR-H2, and a CDR-H3 comprising the sequences set forth in SEQ ID NOS: 35, 36, and 37, respectively, and V_{L} comprises a CDR-L1, a CDR-L2, and a CDR-L3 comprising the sequences set forth in SEQ ID NOS: 38, 39, and 40, respectively.

158. The antibody or antigen-binding fragment thereof of any of claims 149, 150 and 157, wherein the V_{H} region comprises a CDR-H1, a CDR-H2, and a CDR-H3 comprising the sequence set forth in SEQ ID NOS: 16, 17, and 18, respectively, and the V_{L} region comprises a CDR-L1, a CDR-L2, and a CDR-L3 comprising the sequence set forth in SEQ ID NOS: 19, 20, and 21, respectively.

159. The antibody or antigen-binding fragment thereof of any of claims 149, 151 and 157, wherein the V_{H} region comprises a CDR-H1, a CDR-H2, and a CDR-H3 comprising the sequence set forth in SEQ ID NOS: 22, 23, and 24, respectively, and the V_{L} region comprises a CDR-L1, a CDR-L2, and a CDR-L3 comprising the sequence set forth in SEQ ID NOS: 25, 26, and 27, respectively.

160. The antibody or antigen-binding fragment thereof of any of claims 149, 152 and 157, wherein the V_{H} region comprises a CDR-H1, a CDR-H2, and a CDR-H3 comprising the sequence set forth in SEQ ID NOS: 28, 29, and 30, respectively, and the V_{L} region comprises a CDR-L1, a CDR-L2, and a CDR-L3 comprising the sequence set forth in SEQ ID NOS: 31, 26, and 27, respectively.

161. The antibody or antigen-binding fragment thereof of any of claims 149, 153 and 157, wherein the V_{H} region comprises a CDR-H1, a CDR-H2, and a CDR-H3 comprising the sequence set forth in SEQ ID NOS: 22, 32, and 24, respectively, and the V_{L} region comprises a CDR-L1, a CDR-L2, and a CDR-L3 comprising the sequence set forth in SEQ ID NOS: 33, 26, and 34, respectively.

162. The antibody or antigen-binding fragment thereof of any of claims 149, 154 and 157, wherein the V_{H} region comprises a CDR-H1, a CDR-H2, and a CDR-H3 comprising the sequence set forth in SEQ ID NOS: 35, 36, and 37, respectively, and the V_{L} region comprises a CDR-L1, a CDR-L2, and a CDR-L3 comprising the sequence set forth in SEQ ID NOS: 38, 39, and 40, respectively.

163. The antibody or antigen-binding portion thereof of claim 149 or claim 157 wherein:
(i) V_{H} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 1, and V_{L} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 2;
(ii) V_{H} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 3, and V_{L} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 4;
(iii) V_{H} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 5, and V_{L} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 6;
(iv) V_{H} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 7, and V_{L} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 8; or
(v) V_{H} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 9, and V_{L} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 10.

164. An antibody or antigen-binding portion thereof that specifically binds BAFF-R, comprising V_{H} and V_{L}, wherein:
(i) V_{H} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 1, and V_{L} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 2;
(ii) V_{H} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 3, and V_{L} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 4;
(iii) V_{H} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 5, and V_{L} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 6;
(iv) V_{H} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 7, and V_{L} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 8; or
(v) V_{H} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 9, and V_{L} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 10.

165. The antibody or antigen-binding portion thereof of any of claims 149, 150, 157, 158, 160, and 164, wherein V_{H} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 1, and V_{L} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 2.

166. The antibody or antigen-binding portion thereof of any of claims 149, 151, 157, 159, 163, and 164, wherein V_{H} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 3, and V_{L} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 4.

167. The antibody or antigen-binding portion thereof of any of claims 149, 152, 157, 160, 163, and 164, wherein V_{H} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 5, and V_{L} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 6.

168. The antibody or antigen-binding portion thereof of any of claims 149, 153, 157, 161, 163, and 164, wherein V_{H} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 7, and V_{L} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 8.

169. The antibody or antigen-binding portion thereof of any of claims 149, 154, 157, 162, 163, and 164, wherein V_{H} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 9, and V_{L} is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 10.

170. The antibody or antigen-binding portion thereof of any of claims 149, 157, 163, and 164, wherein:
(i) V_{H} and V_{L} are or comprise the sequence set forth in SEQ ID NOS: 1 and 2, respectively;
(ii) V_{H} and V_{L} are or comprise the sequence set forth in SEQ ID NOS: 3 and 4, respectively;
(iii) V_{H} and V_{L} are or comprise the sequence set forth in SEQ ID NOS: 5 and 6, respectively;
(iv) V_{H} and V_{L} are or comprise the sequence set forth in SEQ ID NOS: 7 and 8, respectively; or
(v) V_{H} and V_{L} are or comprise the sequence set forth in SEQ ID NOS: 9 and 10, respectively.

171. An antibody or antigen-binding portion thereof that specifically binds BAFF-R, comprising V_{H} and V_{L}, wherein:
(i) V_{H} and V_{L} are or comprise the sequence set forth in SEQ ID NOS: 1 and 2, respectively;
(ii) V_{H} and V_{L} are or comprise the sequence set forth in SEQ ID NOS: 3 and 4, respectively;
(iii) V_{H} and V_{L} are or comprise the sequence set forth in SEQ ID NOS: 5 and 6, respectively;
(iv) V_{H} and V_{L} are or comprise the sequence set forth in SEQ ID NOS: 7 and 8, respectively; or
(v) V_{H} and V_{L} are or comprise the sequence set forth in SEQ ID NOS: 9 and 10, respectively.

172. The antibody or antigen-binding portion thereof of any of claims 149, 150, 157, 158, 163, 164, 165, 170, and 171, wherein V_{H} and V_{L} are or comprise the sequence set forth in SEQ ID NOS: 1 and 2, respectively.

173. The antibody or antigen-binding portion thereof of any of claims 149, 151, 157, 159, 163, 164, 166, 170, and 171, wherein V_{H} and V_{L} are or comprise the sequence set forth in SEQ ID NOS: 3 and 4, respectively.

174. The antibody or antigen-binding portion thereof of any of claims 149, 152, 157, 160, 163, 164, 167, 170, and 171, wherein V_{H} and V_{L} are or comprise the sequence set forth in SEQ ID NOS: 5 and 6, respectively.

175. The antibody or antigen-binding portion thereof of any of claims 149, 153, 157, 161, 163, 164, 168, 170, and 171, wherein V_{H} and V_{L} are or comprise the sequence set forth in SEQ ID NOS: 7 and 8, respectively.

176. The antibody or antigen-binding portion thereof of any of claims 149, 154, 157, 162, 163, 164, 169, 170, and 171, wherein V_{H} and V_{L} are or comprise the sequence set forth in SEQ ID NOS: 9 and 10, respectively.

177. The antibody or antigen-binding portion thereof of any of claims 149-176, wherein the antibody is a full-length antibody.

178. The antibody or antigen-binding portion thereof of any of claims 149-176, wherein the antibody is an antigen-binding fragment.

179. The antibody or antigen-binding portion thereof of any of claims 149-178, wherein said anti-BAFF-R antibody or antigen-binding portion thereof is recombinant.

180. The antibody or antigen-binding portion thereof of any of claims 149-179, wherein V_{H} and V_{L} are human or are derived from a human protein.

181. The antibody or antigen-binding portion thereof of any of claims 149-176 and 177-180, wherein the antigen-binding portion thereof comprises a single chain variable fragment (scFv).

182. The antibody or antigen-binding portion thereof of claim 181, wherein V_{H} is amino-terminal to V_{L}.

183. The antibody or antigen-binding portion thereof of claim 181, wherein V_{H} is carboxy-terminal to V_{L}.

184. The antibody or antigen-binding portion thereof of any of claims 179-181, when V_{H} and V_{L} are joined by a flexible linker.

185. The antibody or antigen-binding portion thereof of claim 184, wherein the flexible linker comprises the sequence set forth in SEQ ID NO: 58.

186. The antibody or antigen-binding portion thereof of any of claims 179-185, wherein the antigen-binding portion is or comprises the sequence set forth in SEQ ID NO: 11, 12, 13, 14, or 15, or an amino acid sequence having at least at or about 85%, 86%, 87%, 88%, 89%, 90%, 91%, 92%, 93%, 94%, 95%, 96%, 97%, 98% or 99% sequence identity to SEQ ID NO: 11, 12, 13, 14, or 15.

187. The antibody or antigen-binding portion thereof of any of claims 179-185, wherein the antigen-binding portion is or comprises the sequence set forth in SEQ ID NO: 11.

188. The antibody or antigen-binding portion thereof of any of claims 179-185, wherein the antigen-binding portion is or comprises the sequence set forth in SEQ ID NO: 12.

189. The antibody or antigen-binding portion thereof of any of claims 179-185, wherein the antigen-binding portion is or comprises the sequence set forth in SEQ ID NO: 13.

190. The antibody or antigen-binding portion thereof of any of claims 179-185, wherein the antigen-binding portion is or comprises the sequence set forth in SEQ ID NO: 14.

191. The antibody or antigen-binding portion thereof of any of claims 179-185, wherein the antigen-binding portion is or comprises the sequence set forth in SEQ ID NO: 15.

192. The antibody or antigen-binding portion thereof of any of claims 149-191, wherein said anti-BAFF-R antibody or antigen-binding portion thereof specifically binds to a human B-cell activating factor receptor (BAFF-R) protein.

193. The antibody or antigen-binding portion thereof of claim 192, wherein the human BAFF-R protein comprises an amino acid sequence set forth in SEQ ID NO: 120.

194. A pharmaceutical composition comprising the antibody or antigen-binding portion of any of claims 149-193, and a pharmaceutical carrier.

195. A chimeric antigen receptor (CAR) comprising an extracellular binding domain comprising an antibody or antigen-binding portion thereof of any of claims 149-193, a transmembrane domain, and an intracellular signaling domain.

196. The CAR of claim 195, wherein the intracellular signaling domain comprises an intracellular signaling domain capable of inducing a primary activation signal in a T cell.

197. The CAR of claim 195 or 196, wherein the intracellular signaling domain is a domain from a T cell receptor (TCR) component and/or comprises an immunoreceptor tyrosine-based activation motif (ITAM).

198. The CAR of any one of claims 195-197, wherein the intracellular signaling domain is a cytoplasmic signaling domain of a CD3-zeta (CD3ζ) chain, optionally a human CD3ζ chain.

199. The CAR of any one of claims 195-198, wherein the intracellular signaling region further comprises a costimulatory signaling region.

200. The CAR of claim 199, wherein the costimulatory signaling region is between the transmembrane region and the intracellular signaling domain.

201. The CAR of claim 199 or claim 200, wherein the costimulatory signaling region comprises an intracellular signaling domain of a T cell costimulatory molecule or a signaling portion thereof.

202. The CAR of any one of claims 199-201, wherein the costimulatory signaling region comprises an intracellular signaling domain of 4-1BB, optionally a human 4-1BB.

203. A conjugate, comprising the antibody or antigen-binding portion thereof of any of claims 149-193 and a heterologous molecule or moiety.

204. The conjugate of claim 203, wherein the heterologous molecule or moiety is a therapeutic moiety.

205. A nucleic acid encoding the antibody or antigen-binding portion of any of claims 149-193.

206. A polynucleotide comprising a nucleic acid of claim 205.

207. A polynucleotide comprising a nucleic acid encoding the conjugate of claim 203 or claim 204.

208. The polynucleotide of claim 206 or claim 207, wherein the polynucleotide is optimized by splice site elimination.

209. The polynucleotide of any one of claims 206-208, wherein the polynucleotide is codon-optimized for expression in a human cell.

210. An expression vector comprising the nucleic acid of claim 205.

211. A vector, comprising the polynucleotide of any of claims 206-209.

212. The vector of claim 211, wherein the vector is a viral vector.

213. The vector of claim 212, wherein the viral vector is a retroviral vector or a lentiviral vector.

214. A cell comprising the antibody or antigen-binding portion thereof of any of claims 149-193, the CAR of any one of claims 195-202, or the conjugate of claim 203 or claim 204.

215. A cell comprising the polynucleotide of any of claims 206-209, or the vector of any of claims 210-213.

216. The cell of claim 215, wherein the cell is an immune cell.

217. The cell of claim 215 or claim 216, that is a lymphocyte.

218. The cell of any one of claims 215-217, that is an NK cell or a T cell.

219. The cell of any one of claims 215-218, wherein the cell is a T cell and the T cell is a CD4+ T cell or a CD8+ T cell.

220. The cell of any of claims 214-219, wherein the cell is a primary cell obtained from a subject.

221. A composition comprising the cell of any of claims 214-220.

222. A composition comprising the antibody or antigen-binding portion thereof of any of claims 149-193, the CAR of any one of claims 195-202, or the conjugate of claim 203 or claim 204.

223. The composition of claim 212 or claim 213, further comprising a pharmaceutically acceptable excipient.

224. The composition of claim 221 or claim 222, wherein the composition comprises CD4+ and CD8+ T cells and the ratio of CD4+ to CD8+ T cells is from at or about 1:3 to 3:1, optionally at or about 1:2 to 2:1, optionally at or about 1:1.

225. A method of producing an antibody or antigen-binding portion that specifically binds to BAFF-R, comprising culturing the host cell of claim 215 under suitable conditions, and obtaining the product expressed by the host cell.

226. A method for preparing a BAFF-R-targeting drug, an anti-BAFF-R antibody-drug conjugate (ADC), a multifunctional anti-BAFF-R antibody, a reagent for diagnosing a tumor expressing BAFF-R, or an anti-BAFF-R chimeric antigen receptor (CAR) modified immune cell, wherein the method comprises providing the antibody or antigen-binding portion of any of claims 149-193 and incorporating said antibody or antigen-binding portion into the BAFF-R-targeting drug, the anti-BAFF-R ADC, the multifunctional anti-BAFF-R antibody, the reagent for diagnosing a tumor expressing BAFF-R, or the anti-BAFF-R chimeric antigen receptor (CAR) modified immune cell.

227. A method of treatment, comprising administering the cell of any one of claims 214-220 or the composition of any one of claims 221-224 to a subject having a disease or disorder associated with BAFF-R.

228. The cell of any one of claims 214-220 or the composition of any one of claims 221-224 for use in treating a disease or disorder associated with BAFF-R.

229. Use of the cell of any one of claims 214-220 or the composition of any one of claims 221-224 for the manufacture of a medicament for treating a disease or disorder associated with BAFF-R.

230. Use of the cell of any one of claims 214-220 or the composition of any one of claims 221-224 for the treatment of a disease or disorder associated with BAFF-R.

231. A method of treatment, comprising administering the antibody or antigen-binding portion thereof of any of claims 149-193, the pharmaceutical composition of claim 194, the CAR of any one of claims 195-1202, the conjugate of claim 203 or claim 204, the nucleic acid of claim 205, the polynucleotide of any one of claims 206-209, or the vector of any one of claims 210-213 to a subject having a disease or disorder associated with BAFF-R.

232. The anti-BAFF-R antibody or antigen-binding portion thereof of any of claims 149-193, the pharmaceutical composition of claim 194, the CAR of any one of claims 195-202, the conjugate of claim 203 or claim 204, the nucleic acid of claim 205, the polynucleotide of any one of claims 206-209, or the vector of any one of claims 210-213 for use in treating a disease or disorder associated with BAFF-R.

233. Use of the anti-BAFF-R antibody or antigen-binding portion thereof of any one of claims 149-193, the pharmaceutical composition of claim 194, the CAR of any one of claims 195-202, the conjugate of claim 203 or claim 204, the nucleic acid of claim 205, the polynucleotide of any one of claims 206-209, or the vector of any one of claims 210-213 for the manufacture of a medicament for treating a disease or disorder associated with BAFF-R.

234. Use of the antibody or antigen-binding portion thereof of any one of claims 149-193, the pharmaceutical composition of claim 194, the CAR of any one of claims 195-202, the conjugate of claim 203 or claim 204, the nucleic acid of claim 205, the polynucleotide of any one of claims 206-209, or the vector of any one of claims 210-213 for the treatment of a disease or disorder associated with BAFF-R.

235. The method, the cell, composition, antibody or antigen-binding portion thereof, conjugate, polynucleotide, or vector for use or the use of any of claims 225-234, wherein the disease or disorder associated with BAFF-R is a cancer.

236. The method, the cell, composition, antibody or antigen-binding portion thereof, conjugate, polynucleotide, or vector for use or the use of claim 235, wherein the cancer is a BAFF-R-expressing cancer.

237. A kit comprising the antibody or antigen-binding portion thereof of any of claims 149-193, the pharmaceutical composition of claim 194, the CAR of any one of claims 195-202, the conjugate of claim 203 or claim 204, the nucleic acid of claim 205, the polynucleotide of any one of claims 206-209, or the vector of any one of claims 210-213, the cell of any one of claims 214-220 or the composition of any of claims 221-224, and instructions for use, optionally wherein the instructions are for administering the antibody or antigen-binding portion thereof, the conjugate, the cell, or the composition, optionally in accord with the method, the cell, composition, antibody or antigen-binding portion thereof, conjugate, polynucleotide, or vector for use or the use of any of claims 225-236.

238. An article of manufacture comprising the antibody or antigen-binding portion thereof of any one of claims 149-193, the pharmaceutical composition of claim 194, the CAR of any one of claims 195-202, the conjugate of claim 203 or claim 204, the nucleic acid of claim 205, the polynucleotide of any one of claims 206-209, or the vector of any one of claims 210-213, the cell of any one of claims 214-220, the composition of any one of claims 221-224, or the kit of claim 237.
